(19) United States
(12) Patent Application Publication
(10) Pub. No.: US 2024/0324451 A1
(43) Pub. Date: Sep. 26, 2024

(54) ORGANIC COMPOUND AND ORGANIC ELECTROLUMINESCENT DEVICE INCLUDING SAME

(71) Applicant: MATERIAL SCIENCE CO.,LTD., Seoul (KR)

(72) Inventors: Jie SONG, Seoul (KR); Tae Wan LEE, Seoul (KR); Hani JEON, Seoul (KR); Seok Geun JIN, Seoul (KR)

(21) Appl. No.: 18/638,940

(22) Filed: Apr. 18, 2024

Related U.S. Application Data
(63) Continuation of application No. PCT/KR2022/015930, filed on Oct. 19, 2022.

(30) Foreign Application Priority Data
Oct. 19, 2021 (KR) 10-2021-0139665
Oct. 18, 2022 (KR) 10-2022-0134405

(51) Int. Cl. H10K 85/60; C07D 405/12; C07D 409/12; H10K 50/15

(52) U.S. Cl. CPC H10K 85/636; C07D 405/12; C07D 409/12; H10K 85/633; H10K 50/15; H10K 85/615; H10K 85/624; H10K 85/6572; H10K 85/6574; H10K 85/6576

(57) ABSTRACT

The present invention relates to a novel organic compound and an organic electroluminescent device including the same, and more specifically, provides an organic electroluminescent device which has a low driving voltage and significantly improved device efficiency characteristics and lifetime.

### CROSS-REFERENCE TO RELATED APPLICATION

The present application is a Continuation of PCT application number PCT/KR2022/015930, filed on Oct. 19, 2022, which is based upon and claims the benefit of priorities to Korean Patent Application Nos. 10-2022-0134405, filed on Oct. 18, 2022, and 10-2021-0139665 filed on Oct. 19, 2021, in the Korean Intellectual Property Office. All of the aforementioned applications are hereby incorporated by reference in their entireties.

### TECHNICAL FIELD

The present invention relates to an organic compound and an organic electroluminescent device including the same.

### BACKGROUND ART

Since organic electroluminescent devices have a simpler structure than other flat panel display devices such as current liquid crystal displays (LCDs), plasma display panels (PDPs), and field emission displays (FEDs), various advantages in a manufacturing process, high brightness and excellent viewing angle characteristics, and a fast response speed and a low driving voltage, the organic electroluminescent devices are being actively developed to be used as flat displays, such as wall-mounted TVs, or light sources, such as backlight of displays, lighting, and billboards.

In an organic electroluminescent device, generally, when a voltage is applied, holes injected from a positive electrode and electrons injected from a negative electrode are recombined to form an exciton, which is an electron-hole pair, and the energy of the exciton is transmitted to a light emitting material and converted into light.

To increase the efficiency and stability of the organic electroluminescent device, research for an organic material for a multi-layered organic electroluminescent device has been actively conducted since the low-voltage organic electroluminescent device in which the organic thin film is formed between two opposite electrodes was reported to by C. W. Tang, et al. of Eastman Kodak (C. W. Tang and S. A. Vanslyke, Applied Physics Letters, Volume 51, Pages 913, 1987).

Generally, the organic electroluminescent device has a structure which includes a negative electrode (electron injection electrode), a positive electrode (hole injection electrode), and one or more organic layers between the two electrodes. In this case, in the organic electroluminescent device, a hole injection layer (HIL), a hole transport layer (HTL), a light emitting layer (EML), an electron transport layer (ETL), or an electron injection layer (EIL) may be sequentially stacked from the positive electrode, and to increase the efficiency of the light emitting layer, an electron blocking layer (EBL) or a hole blocking layer (HBL) may be added in front of or behind the light emitting layer.

The reason why the organic electroluminescent device is manufactured in a multi-layer thin film structure is to stabilize an interface between the electrode and the organic material and increase luminous efficiency.

In particular, in the case of organic compounds used as materials for multi-layer thin films, since there is a large difference in movement speeds of holes and electrons according to their characteristics, holes and electrons may be effectively transported to the light emitting layer only when hole transport layers and electron transport layers containing appropriate compunds are used, and thus densities of the holes and the electrons can be balanced to excellently increase luminous efficiency.

Therefore, since the characteristics of the organic compound components contained in each layer of the organic thin film layer not only greatly affects a driving voltage, luminous efficiency, luminance, and lifetime of the device, but also affect the efficiency or lifetime of a finally produced display, it is important to use specific organic materials appropriate for the multi-layer structure in the organic electroluminescent devices. Therefore, research for the components included in each layer of the organic thin film layer is actively being conducted.

### DISCLOSURE

### Documents of Related Art

### Patent Documents

(Patent Document 1) KR No. 10-2014-0133572 A1
(Patent Document 2) KR No. 10-1663355 B1

### Technical Problem

The present invention is directed to providing an organic electroluminescent device which includes a novel organic compound and has a low driving voltage and excellent device efficiency characteristics and lifetime characteristics.

### Technical Solution

To achieve the object, the present invention may relate to a compound represented by Chemical Formula 1 below.

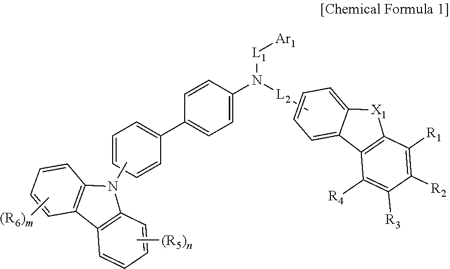

wherein,
n and m are an integer of 0 to 4,
X₁ is selected from the group consisting of O, N(R₇), C(R₈)(R₉), and S,
L₁ and L₂ are the same as or different from each other and are each independently selected from the group consisting of a single bond, a substituted or unsubstituted arylene group having 6 to 30 carbon atoms, a substituted or unsubstituted heteroarylene group having 1 to 30 carbon atoms, a substituted or unsubstituted alkylene group having 1 to 10 carbon atoms, a substituted or unsubstituted cycloalkylene group having 3 to 10 carbon atoms, a substituted or unsubstituted alkenylene group with 2 to 10 carbon atoms, a substituted or unsubstituted cycloalkenylene group having 3 to 10 carbon atoms, a substituted or unsubstituted heteroalkylene group having 1 to 10 carbon atoms, a substituted or unsubstituted heterocycloalkylene group having 2 to 10 carbon atoms, a substituted or unsubstituted heteroalkenylene group having 2 to 10 carbon atoms, and a substituted or unsubstituted heterocycloalkenylene group having 2 to 10 carbon atoms,

An is selected from the group consisting of a substituted or unsubstituted aryl group having 6 to 30 carbon atoms, a substituted or unsubstituted heteroaryl group having 1 to 30 carbon atoms, a substituted or unsubstituted alkyl group having 1 to 20 carbon atoms, a substituted or unsubstituted cycloalkyl group having 3 to 20 carbon atoms, a substituted or unsubstituted heteroalkyl group having 1 to 20 carbon atoms, a substituted or unsubstituted heterocycloalkyl group having 2 to 20 carbon atoms, a substituted or unsubstituted aralkyl group having 6 to 30 carbon atoms, a substituted or unsubstituted alkenyl group having 2 to 20 carbon atoms, a substituted or unsubstituted cycloalkenyl group having 3 to 20 carbon atoms, and a substituted or unsubstituted heteroalkenyl groups having 2 to 20 carbon atoms,

R₁ to R₉ are the same as or different from each other, are each independently selected from the group consisting of hydrogen, deuterium, a cyano group, a nitro group, a halogen group, a hydroxy group, a substituted or unsubstituted alkylthio group having 1 to 4 carbon atoms, a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted alkynyl group having 2 to 24 carbon atoms, a substituted or unsubstituted aralkyl group having 7 to 30 carbon atoms, a substituted or unsubstituted aryl group having 6 to 30 carbon atoms, a substituted or unsubstituted heteroaryl group having 1 to 60 carbon atoms, a substituted or unsubstituted heteroarylalkyl group having 2 to 30 carbon atoms, a substituted or unsubstituted alkoxy group having 1 to 30 carbon atoms, a substituted or unsubstituted alkylamino group having 1 to 30 carbon atoms, a substituted or unsubstituted arylamino group having 6 to 30 carbon atoms, a substituted or unsubstituted aralkylamino group having 7 to 30 carbon atoms, a substituted or unsubstituted heteroarylamino group having 1 to 24 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms, and are bonded with adjacent groups to form a substituted or unsubstituted ring, and
any one or more of R₁ to R₄ is bonded with adjacent groups to form a substituted or unsubstituted ring.

In addition, the present invention relates to an organic electroluminescent device including a first electrode, a second electrode facing the first electrode, and one or more organic layer interposed between the first electrode and the second electrode, wherein the one or more organic layers include the compound represented by by Chemical Formula 1.

In the present invention, “hydrogen” is hydrogen, light hydrogen, deuterium, or tritium, unless otherwise specified.

In the present invention, “halogen group” indicates fluorine, chlorine, bromine, or iodine.

In the present invention, “alkyl” indicates a monovalent substituent derived from a linear or branched saturated hydrocarbon having 1 to 40 carbon atoms. Examples thereof include methyl, ethyl, propyl, isobutyl, sec-butyl, pentyl, iso-amyl, hexyl, and the like, but are not limited thereto.

In the present invention, “alkenyl” indicates a monovalent substituent derived from a linear or branched unsaturated hydrocarbon having one or more carbon-carbon double bonds and 2 to 40 carbon atoms. Examples thereof include vinyl, allyl, isopropenyl, 2-butenyl, and the like, but are not limited thereto.

In the present invention, “alkynyl” indicates a monovalent substituent derived from a linear or branched unsaturated hydrocarbon having one or more carbon-carbon triple bond and 2 to 40 carbon atoms. Examples thereof include ethynyl, 2-propynyl, and the like, but are not limited thereto.

In the present invention, “alkylthio” indicates the above-described alkyl group bonded through a sulfur linkage (—S—).

In the present invention, “aryl” indicates a monovalent substituent derived from an aromatic hydrocarbon having 6 to 60 carbon atoms, which is a single ring or a combination of two or more rings. In addition, forms in which two or more rings are simply pendant or condensed, specifically, a naphthyl group, an anthracenyl group, a phenanthryl group, a triphenyl group, a pyrenyl group, a phenalenyl group, a perylenyl group, a chrysenyl group, a fluorenyl group, and the like may also be included, but the present invention is not limited thereto. The fluorenyl group may be substituted, and adjacent groups may be bonded to form a ring.

In the present invention, “heteroaryl” indicates a monovalent substituent derived from a monoheterocyclic or polyheterocyclic aromatic hydrocarbon having 6 to 30 carbon atoms. In this case, one or more carbons, preferably, 1 to 3 carbons of the ring are substituted with a heteroatom such as N, O, S or Se. In addition, a form in which two or more rings are simply pendant or condensed may be included, and a condensed form with an aryl group may also be included. Examples of such heteroaryls may include 6-membered monocyclic rings such as pyridyl, pyrazinyl, pyrimidinyl, pyridazinyl, and triazinyl, polycyclic rings such as phenoxathienyl, indolizinyl, indolyl, purinyl, quinolyl, benzothiazole, and carbazolyl, 2-furanyl, N-imidazolyl, 2-isoxazolyl, 2-pyridinyl, 2-pyrimidinyl, and the like, but are not limited thereto.

In the present invention, “aryloxy” indicates a monovalent substituent represented by RO—, in which R indicates aryl having 6 to 60 carbon atoms. Examples of such aryloxy may include phenyloxy, naphthyloxy, diphenyloxy, and the like, but are not limited thereto.

In the present invention, “alkyloxy” indicates a monovalent substituent represented by R′O—, in which R′ indicates alkyl having 1 to 40 carbon atoms and may include a linear, branched, or cyclic structure. Examples of alkyloxy may include methoxy, ethoxy, n-propoxy, 1-propoxy, t-butoxy, n-butoxy, and pentoxy, but are not limited thereto.

In the present invention, “alkoxy” may be linear, branched chain, or ring chain. The number of carbon atoms of alkoxy is not especially limited, but is preferably 1 to 20. Specifically, the alkoxy may be methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like, but is not limited thereto.

In the present invention, “aralkyl” indicates an aryl-alkyl group in which aryl and alkyl are described above. Preferred aralkyl includes lower alkyl groups. Non-limiting examples of suitable aralkyl groups include benzyl, 2-phenethyl, and naphthalenylmethyl. Bonding to the parent moiety is made via the alkyl.

In the present invention, “arylamino group” indicates an amine substituted with an aryl group having 6 to 30 carbon atoms.

In the present invention, “alkylamino group” indicates an amine substituted with an alkyl group having 1 to 30 carbon atoms.

In the present invention, “aralkyl amino group” indicates an amine substituted with an aryl-alkyl group having 6 to 30 carbon atoms.

In the present invention, “heteroarylamino group” indicates an amine group substituted with an aryl group or heterocyclic group having 6 to 30 carbon atoms.

In the present invention, “heteroaralkyl group” indicates an aryl-alkyl group substituted with a heterocyclic group.

In the present invention, “cycloalkyl” indicates a monovalent substituent derived from a monocyclic or polycyclic non-aromatic hydrocarbon having 3 to 40 carbon atoms. Examples of such cycloalkyl may include cyclopropyl, cyclobutyl, cyclopentyl, cyclohexyl, norbornyl, adamantine, and the like, but are not limited thereto.

In the present invention, “heterocycloalkyl” indicates a monovalent substituent derived from a non-aromatic hydrocarbon having 3 to 40 carbon atoms, and one or more carbons, preferably, 1 to 3 carbons of the ring is substituted with a hetero atom such as N, O, S or Se. Examples of such heterocycloalkyl may include morpholine, piperazine, and the like, but are not limited thereto.

In the present invention, “alkylsilyl” indicates silyl substituted with alkyl having 1 to 40 carbon atoms, and “arylsilyl” indicates silyl substituted with aryl having 6 to 60 carbon atoms.

In the present invention, “condensed ring” indicates a condensed aliphatic ring, a condensed aromatic ring, a condensed heteroaliphatic ring, a condensed heteroaromatic ring, or a combination thereof.

In the present invention, “forming a ring by being bonded with adjacent groups” indicates forming a substituted or unsubstituted aliphatic hydrocarbon ring, a substituted or unsubstituted aromatic hydrocarbon ring, a substituted or unsubstituted aliphatic heterocycle, a substituted or unsubstituted aromatic heterocycle, or a condensation ring thereof by being bonded with adjacent groups.

In the present invention, examples of “aromatic hydrocarbon rings” may include a phenyl group, a naphthyl group, an anthracenyl group and the like, but are not limited thereto.

In the present invention, “aliphatic heterocycle” indicates an aliphatic ring including one or more heteroatoms.

In the present invention, “aromatic heterocycle” indicates an aromatic ring including one or more heteroatoms.

In the present invention, “substitution” indicates substituting a hydrogen atom bonded to a carbon atom of a compound with another substituent, and a location to be substituted is not limited as long as it is a location at which the hydrogen atom is substituted, that is, a location at which the substituent can be substituted, and when two or more substituents are substituted, the two or more substituents may be the same as or different from each other. The substituent may be substituted with one or more substituents selected from the group consisting of hydrogen, a cyano group, a nitro group, a halogen group, a hydroxy group, an alkyl group having 1 to 30 carbon atoms, an alkenyl group having 2 to 30 carbon atoms, an alkynyl group having 2 to 24 carbon atoms, a heteroalkyl group having 2 to 30 carbon atoms, an aralkyl group having 6 to 30 carbon atoms, an aryl group having 5 to 30 carbon atoms, a heteroaryl group having 2 to 30 carbon atoms, a heteroarylalkyl group having 3 to 30 carbon atoms, an alkoxy group having 1 to 30 carbon atoms, an alkylamino group having 1 to 30 carbon atoms, an arylamino group having 6 to 30 carbon atoms, an aralkylamino group having 6 to 30 carbon atoms, a heteroarylamino group having 2 to 24 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms, but is not limited to the above examples.

### Advantageous Effects

The present invention can provide the novel organic compound and can have excellent interface characteristics with adjacent layers and excellent chemical stability when used as a material for an organic electroluminescent device.

In addition, the present invention can provide the organic electroluminescent device which includes the novel organic compound and has a low driving voltage and excellent device efficiency characteristics and lifetime characteristics.

### MODE FOR INVENTION

Hereinafter, embodiments of the present invention will be described in detail so that those skilled in the art can easily carry out the present invention. However, the present invention may be implemented in various different forms and is not limited to the embodiments described herein.

A novel organic compound according to the present invention may have excellent interface characteristics with adjacent layers and excellent chemical stability, and in particular, have a HOMO energy level at which hole transport is easy and thus may be used as a material for a hole transport auxiliary layer of the organic electroluminescent device with excellent hole transport characteristics to a light emitting layer.

Specifically, the compound represented by Chemical Formula 1 below is as follows:

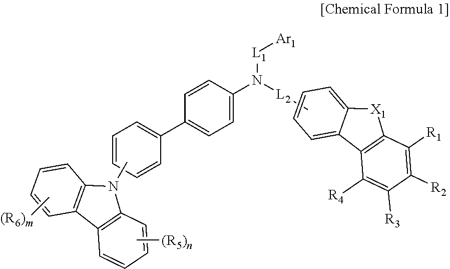

wherein,
n and m are an integer of 0 to 4,
X₁ is selected from the group consisting of O, N(R₇), C(R₈)(R₉), and S,
L₁ and L₂ are the same as or different from each other and are each independently selected from the group consisting of a single bond, a substituted or unsubstituted arylene group having 6 to 30 carbon atoms, a substituted or unsubstituted heteroarylene group having 1 to 30 carbon atoms, a substituted or unsubstituted alkylene group having 1 to 10 carbon atoms, a substituted or unsubstituted cycloalkylene group having 3 to 10 carbon atoms, a substituted or unsubstituted alkenylene group with 2 to 10 carbon atoms, a substituted or unsubstituted cycloalkenylene group having 3 to 10 carbon atoms, a substituted or unsubstituted heteroalkylene group having 1 to 10 carbon atoms, a substituted or unsubstituted heterocycloalkylene group having 2 to 10 carbon atoms, a substituted or unsubstituted heteroalkenylene group having 2 to 10 carbon atoms, and a substituted or unsubstituted heterocycloalkenylene group having 2 to 10 carbon atoms,
Ar₁ is selected from the group consisting of a substituted or unsubstituted aryl group having 6 to 30 carbon atoms, a substituted or unsubstituted heteroaryl group having 1 to 30 carbon atoms, a substituted or unsubstituted alkyl group having 1 to 20 carbon atoms, a substituted or unsubstituted cycloalkyl group having 3 to 20 carbon atoms, a substituted or unsubstituted heteroalkyl group having 1 to 20 carbon atoms, a substituted or unsubstituted heterocycloalkyl group having 2 to 20 carbon atoms, a substituted or unsubstituted aralkyl group having 6 to 30 carbon atoms, a substituted or unsubstituted alkenyl group having 2 to 20 carbon atoms, a substituted or unsubstituted cycloalkenyl group having 3 to 20 carbon atoms, and a substituted or unsubstituted heteroalkenyl groups having 2 to 20 carbon atoms,
R₁ to R₉ are the same as or different from each other, are each independently selected from the group consisting of hydrogen, deuterium, a cyano group, a nitro group, a halogen group, a hydroxy group, a substituted or unsubstituted alkylthio group having 1 to 4 carbon atoms, a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted alkynyl group having 2 to 24 carbon atoms, a substituted or unsubstituted aralkyl group having 7 to 30 carbon atoms, a substituted or unsubstituted aryl group having 6 to 30 carbon atoms, a substituted or unsubstituted heteroaryl group having 1 to 60 carbon atoms, a substituted or unsubstituted heteroarylalkyl group having 2 to 30 carbon atoms, a substituted or unsubstituted alkoxy group having 1 to 30 carbon atoms, a substituted or unsubstituted alkylamino group having 1 to 30 carbon atoms, a substituted or unsubstituted arylamino group having 6 to 30 carbon atoms, a substituted or unsubstituted aralkylamino group having 7 to 30 carbon atoms, a substituted or unsubstituted heteroarylamino group having 1 to 24 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms, and are bonded with adjacent groups to form a substituted or unsubstituted ring, and

Any one or more of R₁ to R₄ are bonded with adjacent groups to form a substituted or unsubstituted ring.

The compound represented by Chemical Formula 1 may be compounds represented by Chemical Formulas 2 to 7:

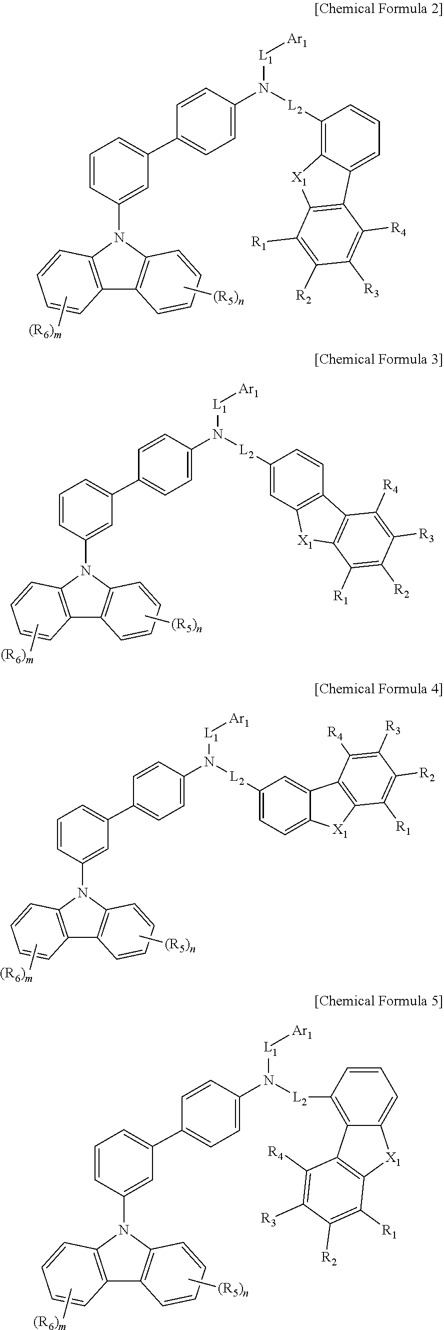

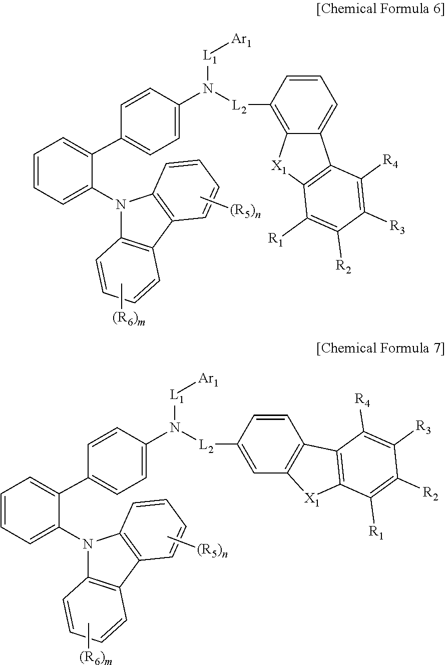

wherein,
n, m, L₁, L₂, Ar₁, X₁, and R₁ to R₆ are defined in Chemical Formula 1.

The L₁ and L₂ are the same as or different from each other and are each independently be a single bond or a substituted or unsubstituted arylene group having 6 to 30 carbon atoms.

X₁ is O or S.

Ar₁ is selected from the group consisting of Chemical Formulas 8 to 12 below:

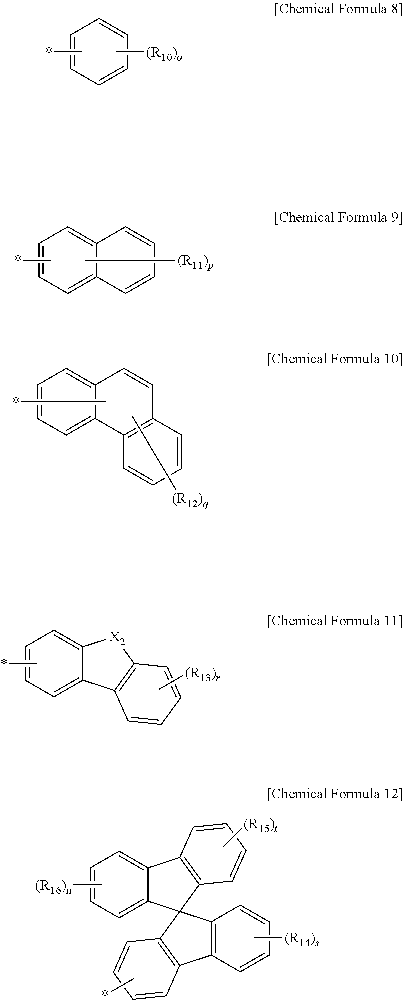

wherein,
* indicates a portion which is bonded,
is an integer from 0 to 5,
p is an integer from 0 to 7,
q is an integer from 0 to 6,
r, s, t, and u are the same as or different from each other and are each independently an integer from 0 to 4,
X₂ is selected from the group consisting of O, N(R₁₇), C(R₁₈)(R₁₉), and S, and

R₁₀ to R₁₉ are the same as or different from each other, are each independently selected from the group consisting of hydrogen, deuterium, a cyano group, a nitro group, a halogen group, a hydroxy group, a substituted or unsubstituted alkylthio group having 1 to 4 carbon atoms, a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted alkynyl group having 2 to 24 carbon atoms, a substituted or unsubstituted aralkyl group having 7 to 30 carbon atoms, a substituted or unsubstituted aryl group having 6 to 30 carbon atoms, a substituted or unsubstituted heteroaryl group having 1 to 60 carbon atoms, a substituted or unsubstituted heteroarylalkyl group having 2 to 30 carbon atoms, a substituted or unsubstituted alkoxy group having 1 to 30 carbon atoms, a substituted or unsubstituted alkylamino group having 1 to 30 carbon atoms, a substituted or unsubstituted arylamino group having 6 to 30 carbon atoms, a substituted or unsubstituted aralkylamino group having 7 to 30 carbon atoms, a substituted or unsubstituted heteroarylamino group having 1 to 24 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms, and are bonded with adjacent groups to form a substituted or unsubstituted ring.

The compound represented by Chemical Formula 1 according to the present invention is selected from the group consisting of the following compounds, but is not limited thereto:

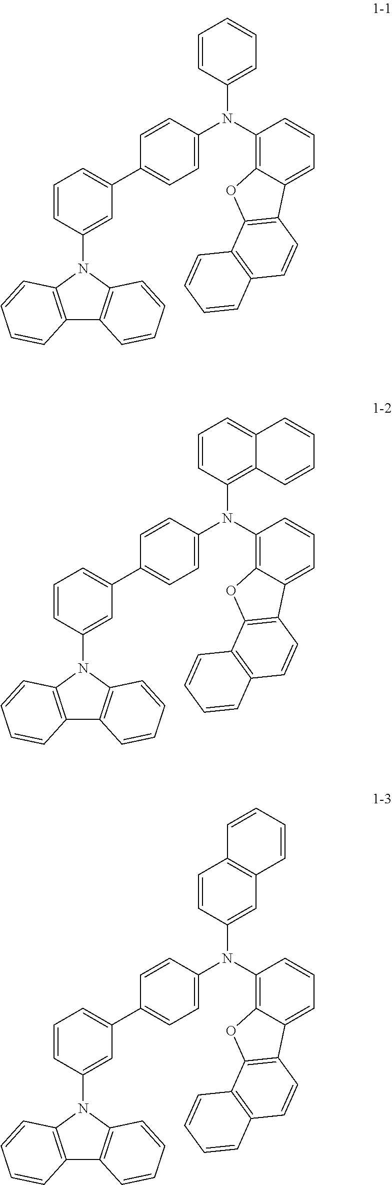 [SMILES: c1ccc(N(c2ccc(-c3cccc(-n4c5ccccc5c5ccccc54)c3)cc2)c2cccc3c2oc2c4ccccc4ccc32)cc1 ; c1cc(-c2ccc(N(c3cccc4ccccc34)c3cccc4c3oc3c5ccccc5ccc43)cc2)cc(-n2c3ccccc3c3ccccc32)c1 ; c1cc(-c2ccc(N(c3ccc4ccccc4c3)c3cccc4c3oc3c5ccccc5ccc43)cc2)cc(-n2c3ccccc3c3ccccc32)c1]

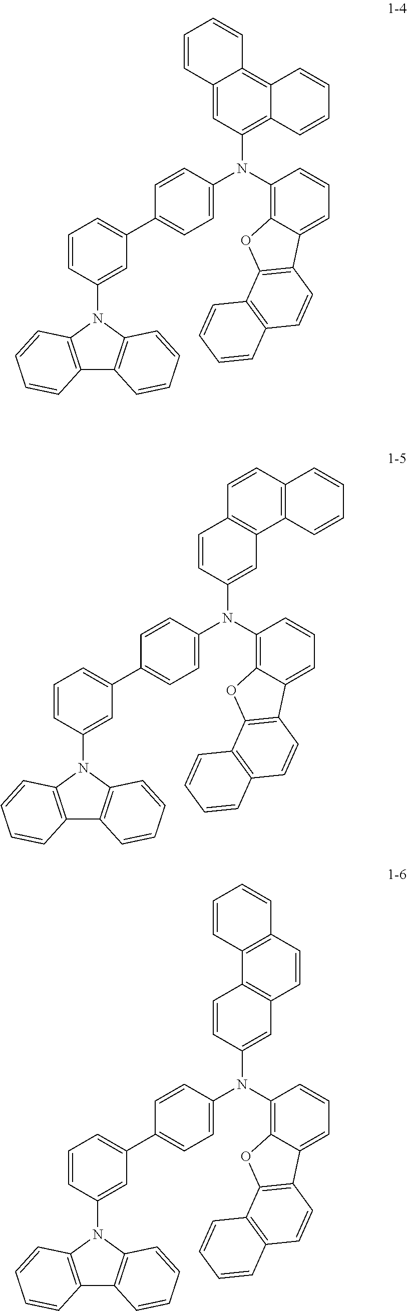 [SMILES: c1cc(-c2ccc(N(c3cc4ccccc4c4ccccc34)c3cccc4c3oc3c5ccccc5ccc43)cc2)cc(-n2c3ccccc3c3ccccc32)c1 ; c1cc(-c2ccc(N(c3ccc4ccc5ccccc5c4c3)c3cccc4c3oc3c5ccccc5ccc43)cc2)cc(-n2c3ccccc3c3ccccc32)c1 ; c1cc(-c2ccc(N(c3ccc4c(ccc5ccccc54)c3)c3cccc4c3oc3c5ccccc5ccc43)cc2)cc(-n2c3ccccc3c3ccccc32)c1]

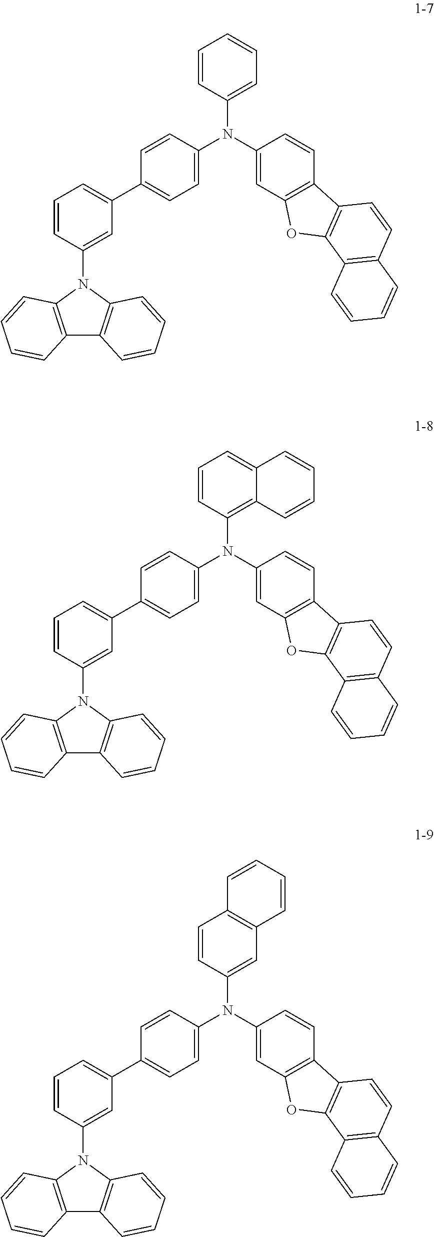 [SMILES: c1ccc(N(c2ccc(-c3cccc(-n4c5ccccc5c5ccccc54)c3)cc2)c2ccc3c(c2)oc2c4ccccc4ccc32)cc1 ; c1cc(-c2ccc(N(c3ccc4c(c3)oc3c5ccccc5ccc43)c3cccc4ccccc34)cc2)cc(-n2c3ccccc3c3ccccc32)c1 ; c1cc(-c2ccc(N(c3ccc4ccccc4c3)c3ccc4c(c3)oc3c5ccccc5ccc43)cc2)cc(-n2c3ccccc3c3ccccc32)c1]

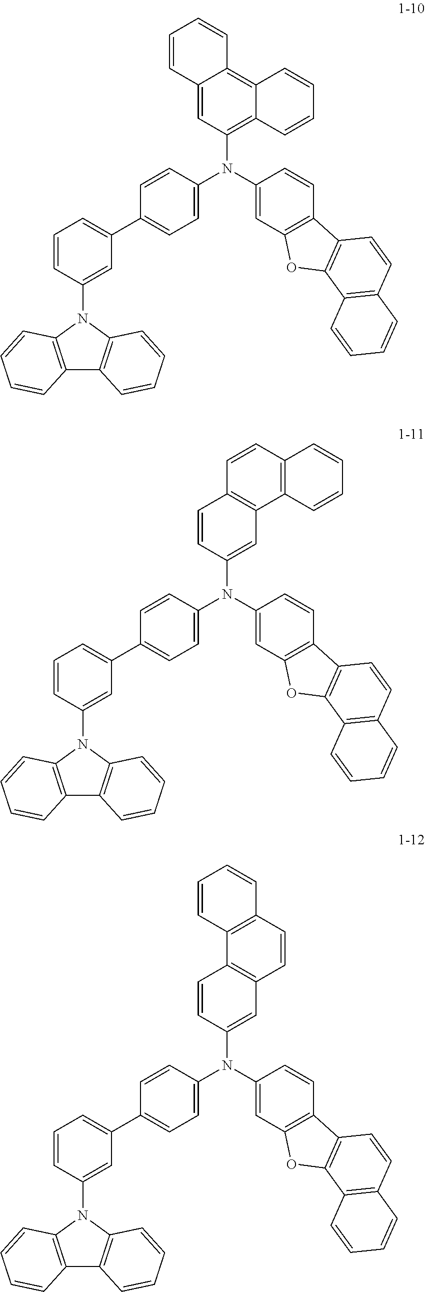 [SMILES: c1cc(-c2ccc(N(c3ccc4c(c3)oc3c5ccccc5ccc43)c3cc4ccccc4c4ccccc34)cc2)cc(-n2c3ccccc3c3ccccc32)c1 ; c1cc(-c2ccc(N(c3ccc4c(c3)oc3c5ccccc5ccc43)c3ccc4ccc5ccccc5c4c3)cc2)cc(-n2c3ccccc3c3ccccc32)c1 ; c1cc(-c2ccc(N(c3ccc4c(ccc5ccccc54)c3)c3ccc4c(c3)oc3c5ccccc5ccc43)cc2)cc(-n2c3ccccc3c3ccccc32)c1]

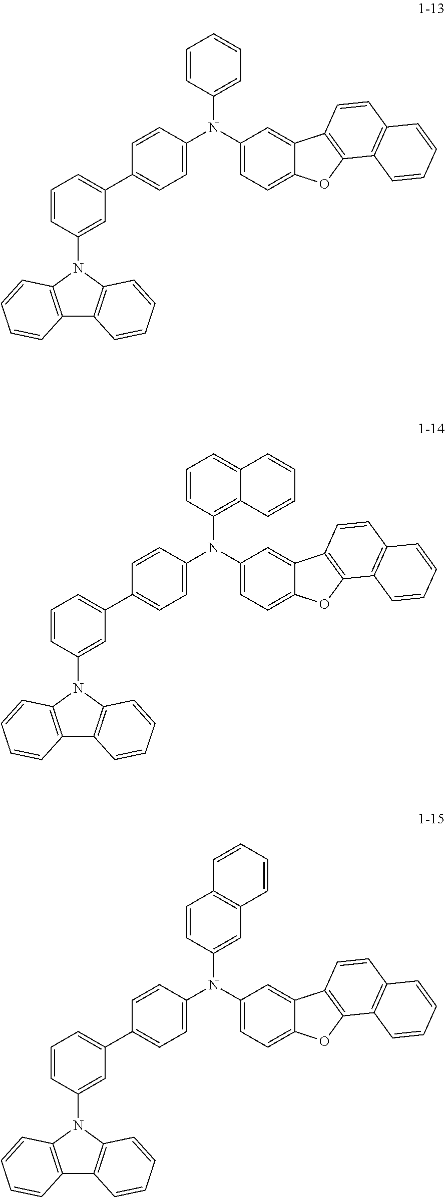 [SMILES: c1ccc(N(c2ccc(-c3cccc(-n4c5ccccc5c5ccccc54)c3)cc2)c2ccc3oc4c5ccccc5ccc4c3c2)cc1 ; c1cc(-c2ccc(N(c3ccc4oc5c6ccccc6ccc5c4c3)c3cccc4ccccc34)cc2)cc(-n2c3ccccc3c3ccccc32)c1 ; c1cc(-c2ccc(N(c3ccc4ccccc4c3)c3ccc4oc5c6ccccc6ccc5c4c3)cc2)cc(-n2c3ccccc3c3ccccc32)c1]

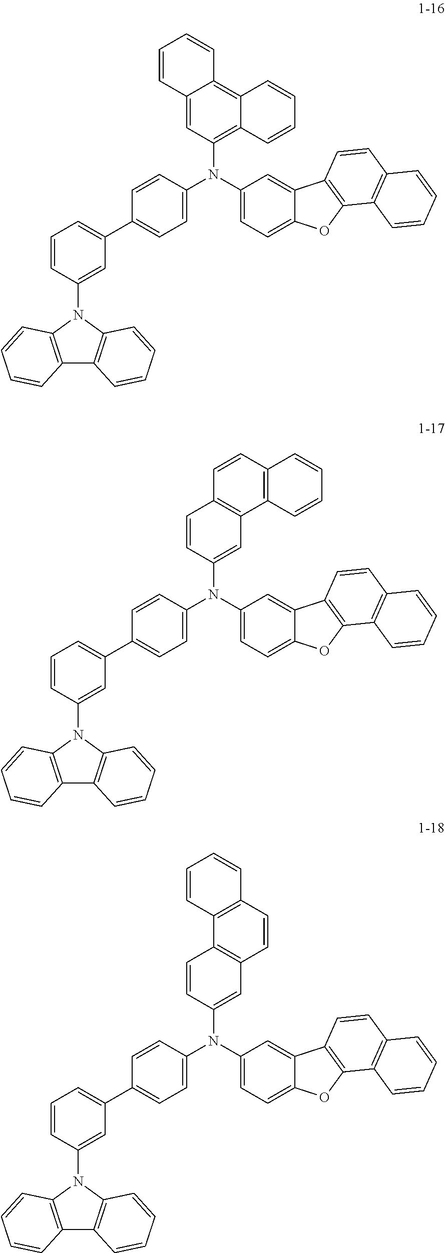 [SMILES: c1cc(-c2ccc(N(c3ccc4oc5c6ccccc6ccc5c4c3)c3cc4ccccc4c4ccccc34)cc2)cc(-n2c3ccccc3c3ccccc32)c1 ; c1cc(-c2ccc(N(c3ccc4ccc5ccccc5c4c3)c3ccc4oc5c6ccccc6ccc5c4c3)cc2)cc(-n2c3ccccc3c3ccccc32)c1 ; c1cc(-c2ccc(N(c3ccc4c(ccc5ccccc54)c3)c3ccc4oc5c6ccccc6ccc5c4c3)cc2)cc(-n2c3ccccc3c3ccccc32)c1]

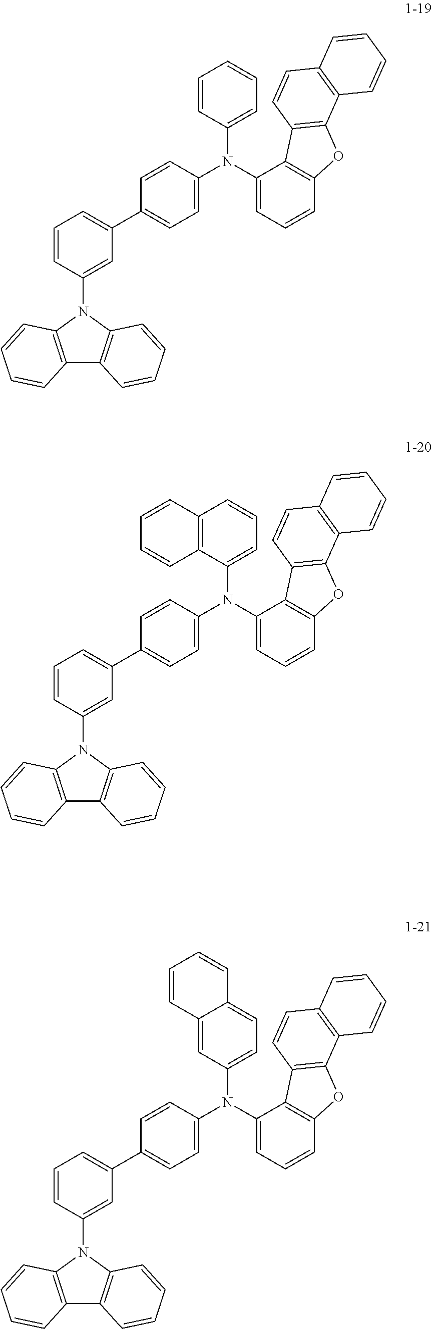 [SMILES: c1ccc(N(c2ccc(-c3cccc(-n4c5ccccc5c5ccccc54)c3)cc2)c2cccc3oc4c5ccccc5ccc4c23)cc1 ; c1cc(-c2ccc(N(c3cccc4ccccc34)c3cccc4oc5c6ccccc6ccc5c34)cc2)cc(-n2c3ccccc3c3ccccc32)c1 ; c1cc(-c2ccc(N(c3ccc4ccccc4c3)c3cccc4oc5c6ccccc6ccc5c34)cc2)cc(-n2c3ccccc3c3ccccc32)c1]

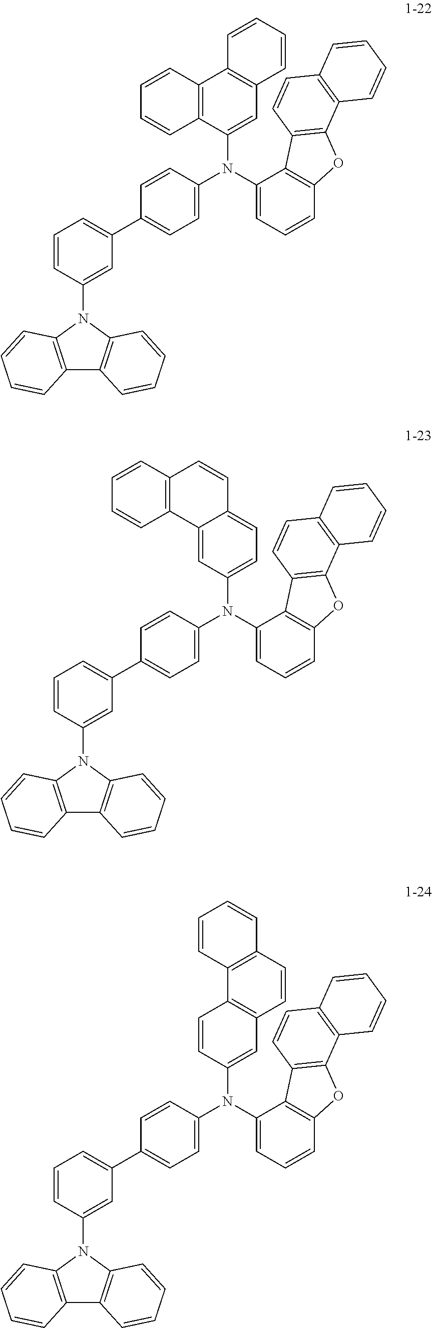 [SMILES: c1cc(-c2ccc(N(c3cc4ccccc4c4ccccc34)c3cccc4oc5c6ccccc6ccc5c34)cc2)cc(-n2c3ccccc3c3ccccc32)c1 ; c1cc(-c2ccc(N(c3ccc4ccc5ccccc5c4c3)c3cccc4oc5c6ccccc6ccc5c34)cc2)cc(-n2c3ccccc3c3ccccc32)c1 ; c1cc(-c2ccc(N(c3ccc4c(ccc5ccccc54)c3)c3cccc4oc5c6ccccc6ccc5c34)cc2)cc(-n2c3ccccc3c3ccccc32)c1]

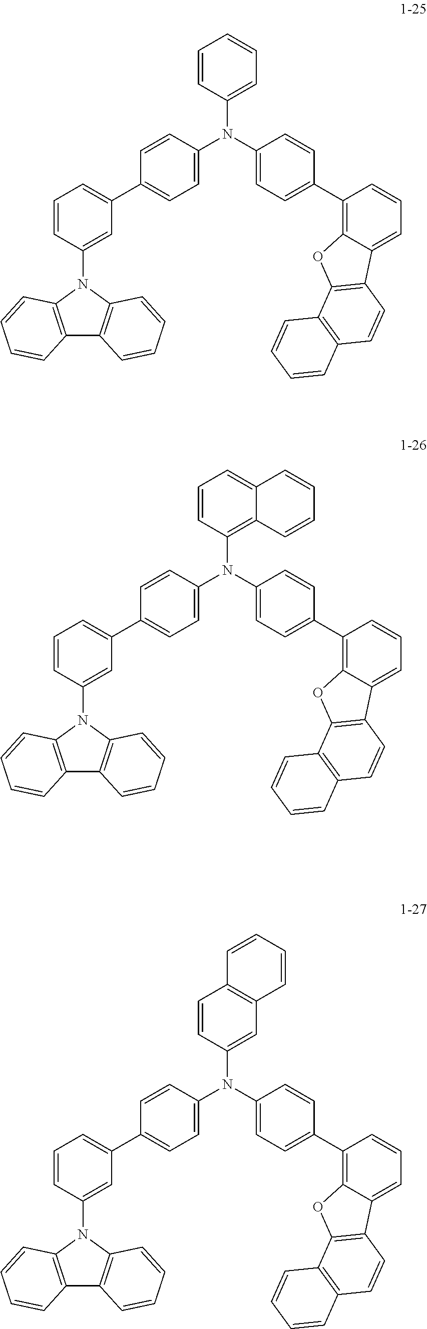 [SMILES: c1ccc(N(c2ccc(-c3cccc(-n4c5ccccc5c5ccccc54)c3)cc2)c2ccc(-c3cccc4c3oc3c5ccccc5ccc43)cc2)cc1 ; c1cc(-c2ccc(N(c3ccc(-c4cccc5c4oc4c6ccccc6ccc54)cc3)c3cccc4ccccc34)cc2)cc(-n2c3ccccc3c3ccccc32)c1 ; c1cc(-c2ccc(N(c3ccc(-c4cccc5c4oc4c6ccccc6ccc54)cc3)c3ccc4ccccc4c3)cc2)cc(-n2c3ccccc3c3ccccc32)c1]

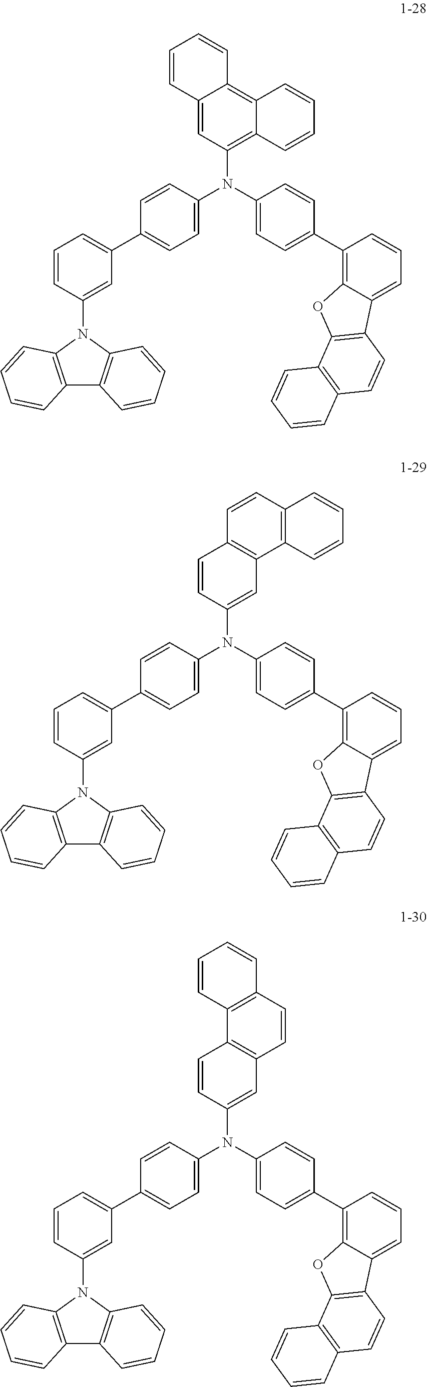 [SMILES: c1cc(-c2ccc(N(c3ccc(-c4cccc5c4oc4c6ccccc6ccc54)cc3)c3cc4ccccc4c4ccccc34)cc2)cc(-n2c3ccccc3c3ccccc32)c1 ; c1cc(-c2ccc(N(c3ccc(-c4cccc5c4oc4c6ccccc6ccc54)cc3)c3ccc4ccc5ccccc5c4c3)cc2)cc(-n2c3ccccc3c3ccccc32)c1 ; c1cc(-c2ccc(N(c3ccc(-c4cccc5c4oc4c6ccccc6ccc54)cc3)c3ccc4c(ccc5ccccc54)c3)cc2)cc(-n2c3ccccc3c3ccccc32)c1]

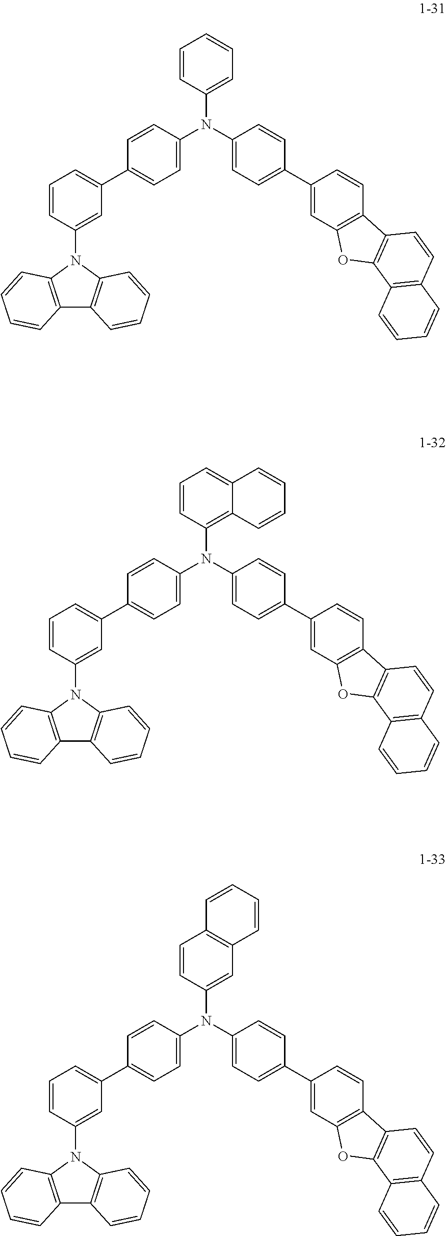 [SMILES: c1ccc(N(c2ccc(-c3cccc(-n4c5ccccc5c5ccccc54)c3)cc2)c2ccc(-c3ccc4c(c3)oc3c5ccccc5ccc43)cc2)cc1 ; c1cc(-c2ccc(N(c3ccc(-c4ccc5c(c4)oc4c6ccccc6ccc54)cc3)c3cccc4ccccc34)cc2)cc(-n2c3ccccc3c3ccccc32)c1 ; c1cc(-c2ccc(N(c3ccc(-c4ccc5c(c4)oc4c6ccccc6ccc54)cc3)c3ccc4ccccc4c3)cc2)cc(-n2c3ccccc3c3ccccc32)c1]

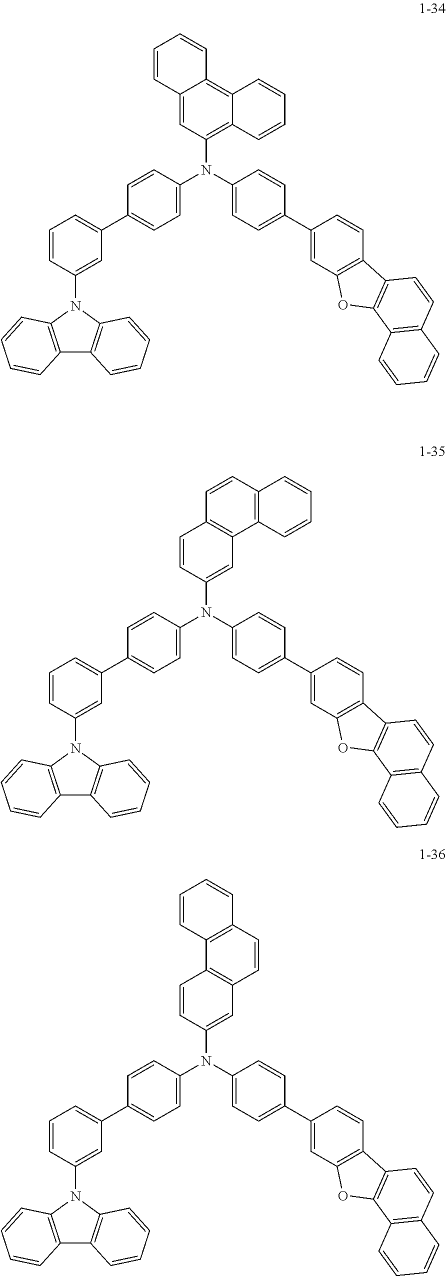 [SMILES: c1cc(-c2ccc(N(c3ccc(-c4ccc5c(c4)oc4c6ccccc6ccc54)cc3)c3cc4ccccc4c4ccccc34)cc2)cc(-n2c3ccccc3c3ccccc32)c1 ; c1cc(-c2ccc(N(c3ccc(-c4ccc5c(c4)oc4c6ccccc6ccc54)cc3)c3ccc4ccc5ccccc5c4c3)cc2)cc(-n2c3ccccc3c3ccccc32)c1 ; c1cc(-c2ccc(N(c3ccc(-c4ccc5c(c4)oc4c6ccccc6ccc54)cc3)c3ccc4c(ccc5ccccc54)c3)cc2)cc(-n2c3ccccc3c3ccccc32)c1]

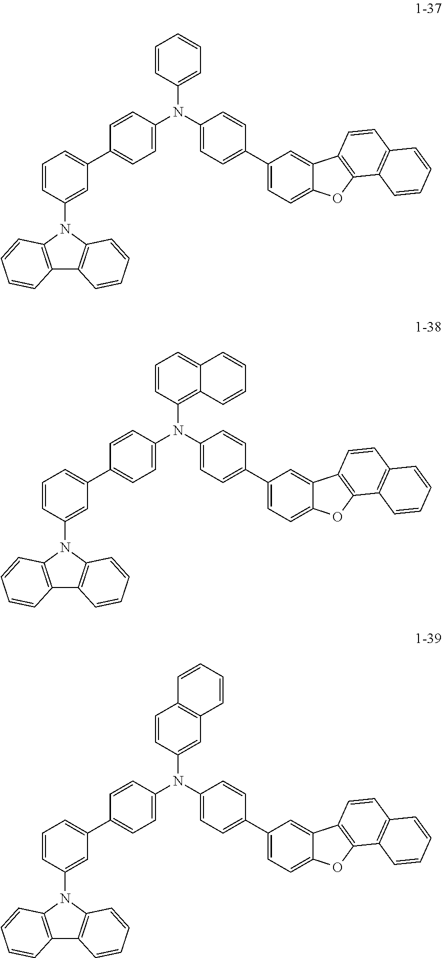 [SMILES: c1ccc(N(c2ccc(-c3cccc(-n4c5ccccc5c5ccccc54)c3)cc2)c2ccc(-c3ccc4oc5c6ccccc6ccc5c4c3)cc2)cc1 ; c1cc(-c2ccc(N(c3ccc(-c4ccc5oc6c7ccccc7ccc6c5c4)cc3)c3cccc4ccccc34)cc2)cc(-n2c3ccccc3c3ccccc32)c1 ; c1cc(-c2ccc(N(c3ccc(-c4ccc5oc6c7ccccc7ccc6c5c4)cc3)c3ccc4ccccc4c3)cc2)cc(-n2c3ccccc3c3ccccc32)c1]

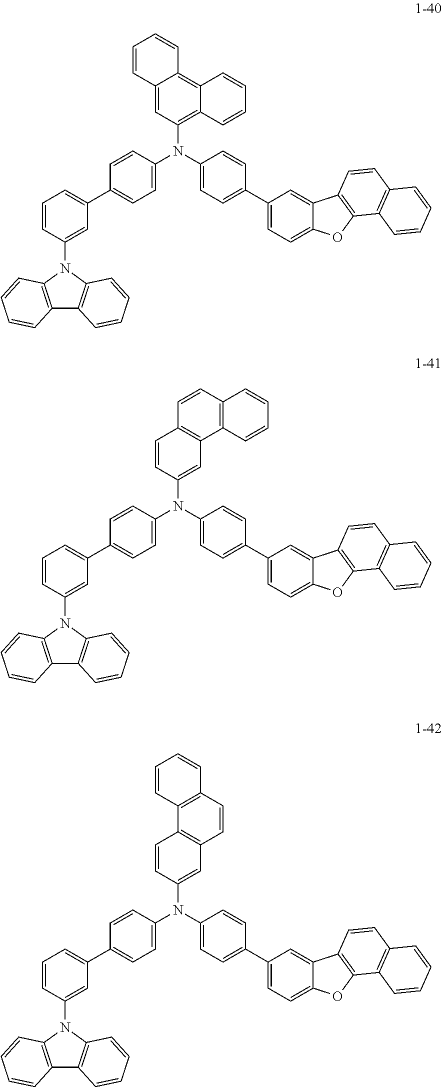 [SMILES: c1cc(-c2ccc(N(c3ccc(-c4ccc5oc6c7ccccc7ccc6c5c4)cc3)c3cc4ccccc4c4ccccc34)cc2)cc(-n2c3ccccc3c3ccccc32)c1 ; c1cc(-c2ccc(N(c3ccc(-c4ccc5oc6c7ccccc7ccc6c5c4)cc3)c3ccc4ccc5ccccc5c4c3)cc2)cc(-n2c3ccccc3c3ccccc32)c1 ; c1cc(-c2ccc(N(c3ccc(-c4ccc5oc6c7ccccc7ccc6c5c4)cc3)c3ccc4c(ccc5ccccc54)c3)cc2)cc(-n2c3ccccc3c3ccccc32)c1]

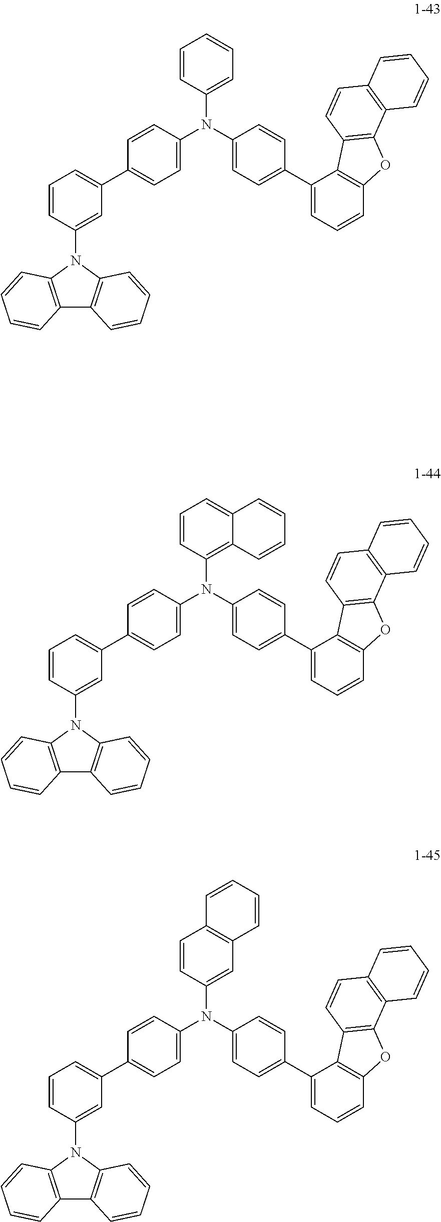 [SMILES: c1ccc(N(c2ccc(-c3cccc(-n4c5ccccc5c5ccccc54)c3)cc2)c2ccc(-c3cccc4oc5c6ccccc6ccc5c34)cc2)cc1 ; c1cc(-c2ccc(N(c3ccc(-c4cccc5oc6c7ccccc7ccc6c45)cc3)c3cccc4ccccc34)cc2)cc(-n2c3ccccc3c3ccccc32)c1 ; c1cc(-c2ccc(N(c3ccc(-c4cccc5oc6c7ccccc7ccc6c45)cc3)c3ccc4ccccc4c3)cc2)cc(-n2c3ccccc3c3ccccc32)c1]

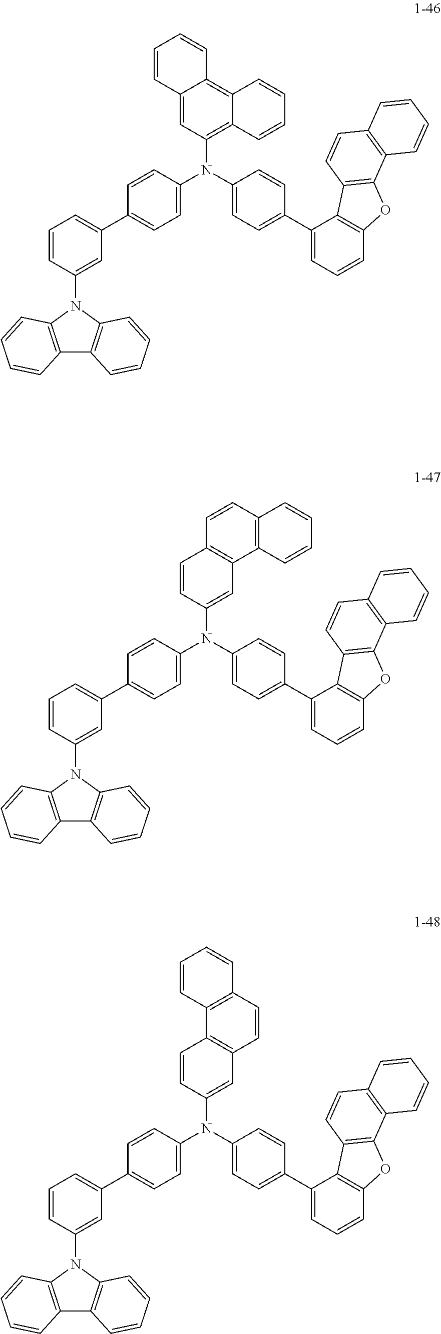 [SMILES: c1cc(-c2ccc(N(c3ccc(-c4cccc5oc6c7ccccc7ccc6c45)cc3)c3cc4ccccc4c4ccccc34)cc2)cc(-n2c3ccccc3c3ccccc32)c1 ; c1cc(-c2ccc(N(c3ccc(-c4cccc5oc6c7ccccc7ccc6c45)cc3)c3ccc4ccc5ccccc5c4c3)cc2)cc(-n2c3ccccc3c3ccccc32)c1 ; c1cc(-c2ccc(N(c3ccc(-c4cccc5oc6c7ccccc7ccc6c45)cc3)c3ccc4c(ccc5ccccc54)c3)cc2)cc(-n2c3ccccc3c3ccccc32)c1]

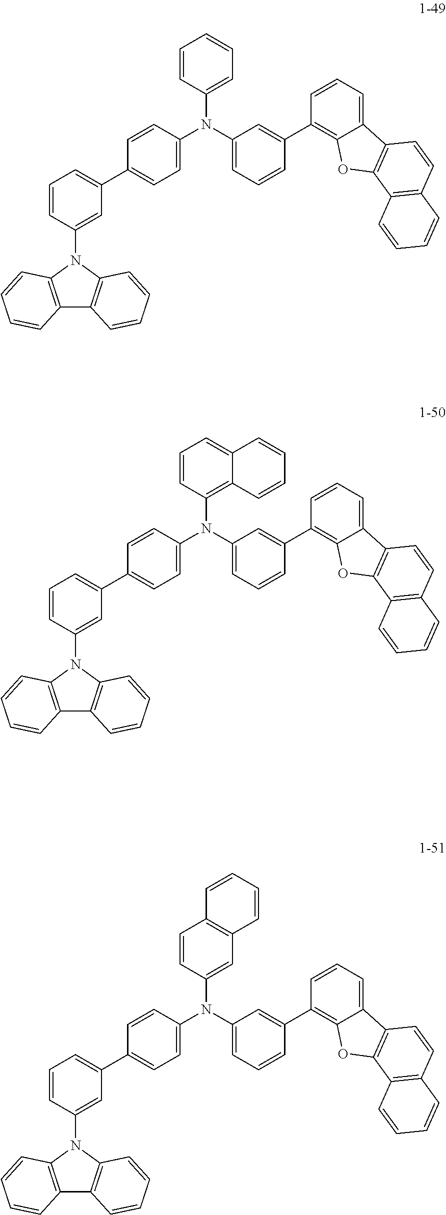 [SMILES: c1ccc(N(c2ccc(-c3cccc(-n4c5ccccc5c5ccccc54)c3)cc2)c2cccc(-c3cccc4c3oc3c5ccccc5ccc43)c2)cc1 ; c1cc(-c2cccc3c2oc2c4ccccc4ccc32)cc(N(c2ccc(-c3cccc(-n4c5ccccc5c5ccccc54)c3)cc2)c2cccc3ccccc23)c1 ; c1cc(-c2cccc3c2oc2c4ccccc4ccc32)cc(N(c2ccc(-c3cccc(-n4c5ccccc5c5ccccc54)c3)cc2)c2ccc3ccccc3c2)c1]

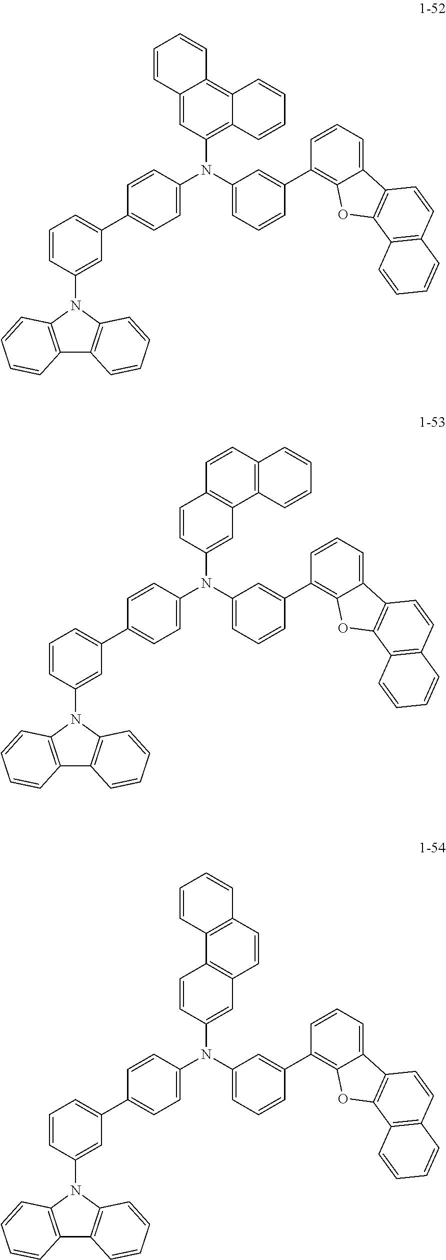 [SMILES: c1cc(-c2cccc3c2oc2c4ccccc4ccc32)cc(N(c2ccc(-c3cccc(-n4c5ccccc5c5ccccc54)c3)cc2)c2cc3ccccc3c3ccccc23)c1 ; c1cc(-c2cccc3c2oc2c4ccccc4ccc32)cc(N(c2ccc(-c3cccc(-n4c5ccccc5c5ccccc54)c3)cc2)c2ccc3ccc4ccccc4c3c2)c1 ; c1cc(-c2cccc3c2oc2c4ccccc4ccc32)cc(N(c2ccc(-c3cccc(-n4c5ccccc5c5ccccc54)c3)cc2)c2ccc3c(ccc4ccccc43)c2)c1]

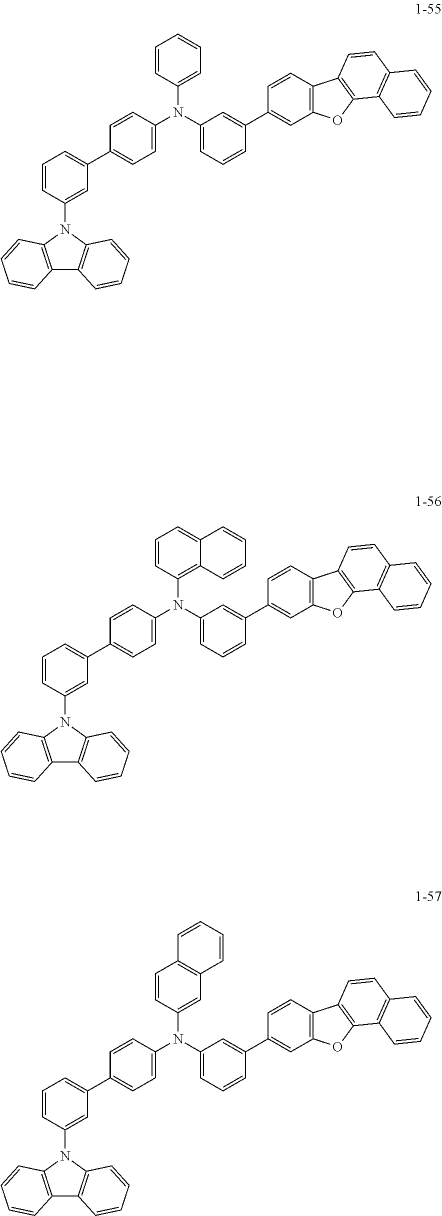 [SMILES: c1ccc(N(c2ccc(-c3cccc(-n4c5ccccc5c5ccccc54)c3)cc2)c2cccc(-c3ccc4c(c3)oc3c5ccccc5ccc43)c2)cc1 ; c1cc(-c2ccc3c(c2)oc2c4ccccc4ccc32)cc(N(c2ccc(-c3cccc(-n4c5ccccc5c5ccccc54)c3)cc2)c2cccc3ccccc23)c1 ; c1cc(-c2ccc3c(c2)oc2c4ccccc4ccc32)cc(N(c2ccc(-c3cccc(-n4c5ccccc5c5ccccc54)c3)cc2)c2ccc3ccccc3c2)c1]

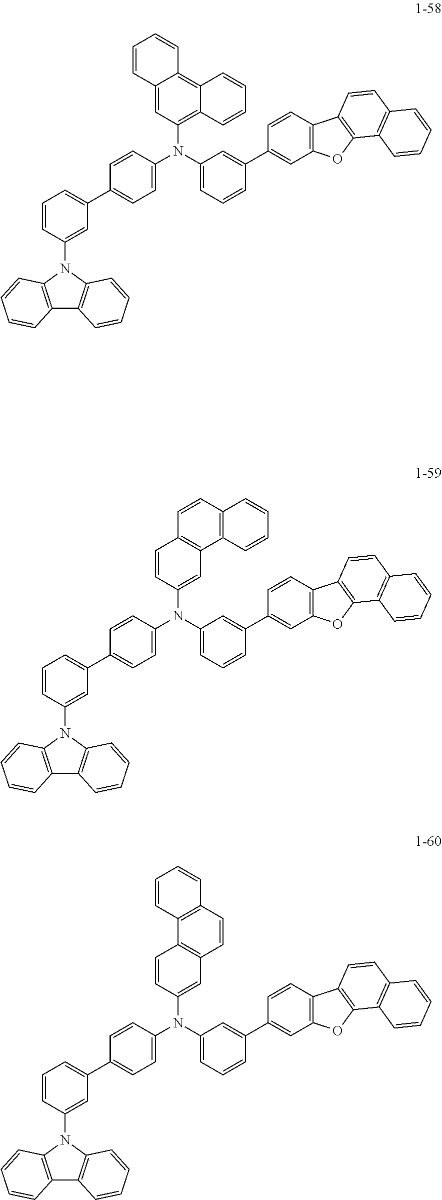 [SMILES: c1cc(-c2ccc3c(c2)oc2c4ccccc4ccc32)cc(N(c2ccc(-c3cccc(-n4c5ccccc5c5ccccc54)c3)cc2)c2cc3ccccc3c3ccccc23)c1 ; c1cc(-c2ccc3c(c2)oc2c4ccccc4ccc32)cc(N(c2ccc(-c3cccc(-n4c5ccccc5c5ccccc54)c3)cc2)c2ccc3ccc4ccccc4c3c2)c1 ; c1cc(-c2ccc3c(c2)oc2c4ccccc4ccc32)cc(N(c2ccc(-c3cccc(-n4c5ccccc5c5ccccc54)c3)cc2)c2ccc3c(ccc4ccccc43)c2)c1]

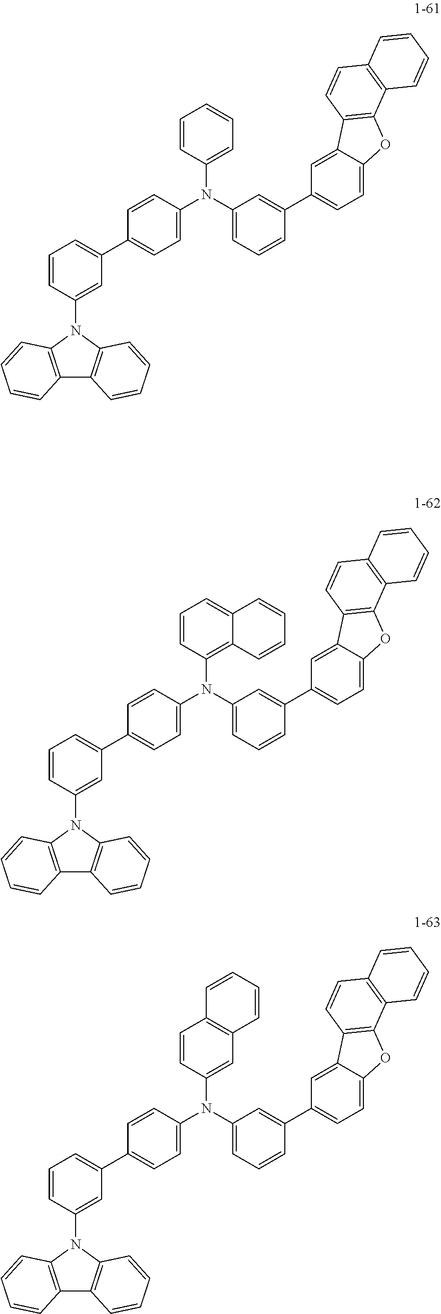 [SMILES: c1ccc(N(c2ccc(-c3cccc(-n4c5ccccc5c5ccccc54)c3)cc2)c2cccc(-c3ccc4oc5c6ccccc6ccc5c4c3)c2)cc1 ; c1cc(-c2ccc3oc4c5ccccc5ccc4c3c2)cc(N(c2ccc(-c3cccc(-n4c5ccccc5c5ccccc54)c3)cc2)c2cccc3ccccc23)c1 ; c1cc(-c2ccc3oc4c5ccccc5ccc4c3c2)cc(N(c2ccc(-c3cccc(-n4c5ccccc5c5ccccc54)c3)cc2)c2ccc3ccccc3c2)c1]

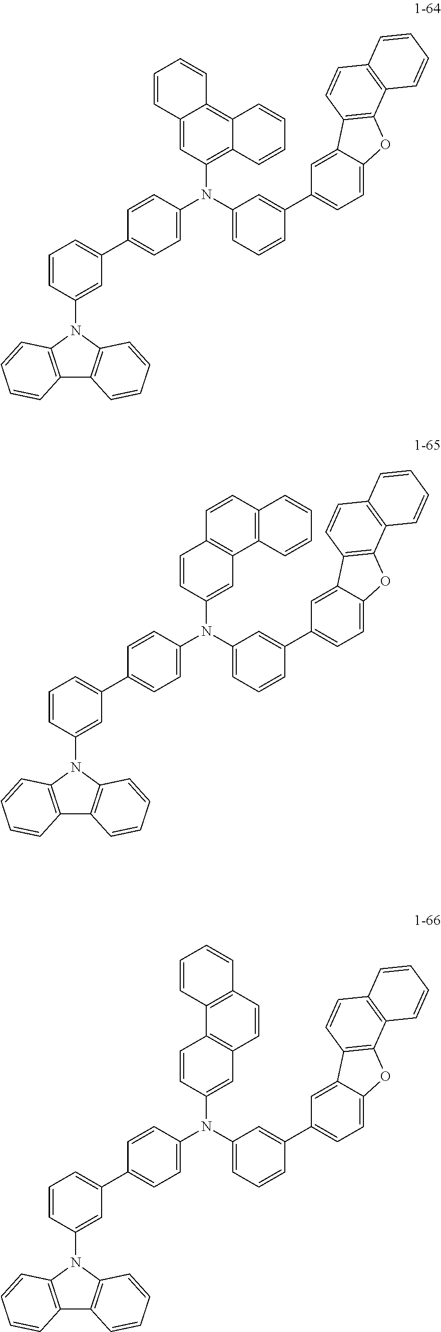 [SMILES: c1cc(-c2ccc3oc4c5ccccc5ccc4c3c2)cc(N(c2ccc(-c3cccc(-n4c5ccccc5c5ccccc54)c3)cc2)c2cc3ccccc3c3ccccc23)c1 ; c1cc(-c2ccc3oc4c5ccccc5ccc4c3c2)cc(N(c2ccc(-c3cccc(-n4c5ccccc5c5ccccc54)c3)cc2)c2ccc3ccc4ccccc4c3c2)c1 ; c1cc(-c2ccc3oc4c5ccccc5ccc4c3c2)cc(N(c2ccc(-c3cccc(-n4c5ccccc5c5ccccc54)c3)cc2)c2ccc3c(ccc4ccccc43)c2)c1]

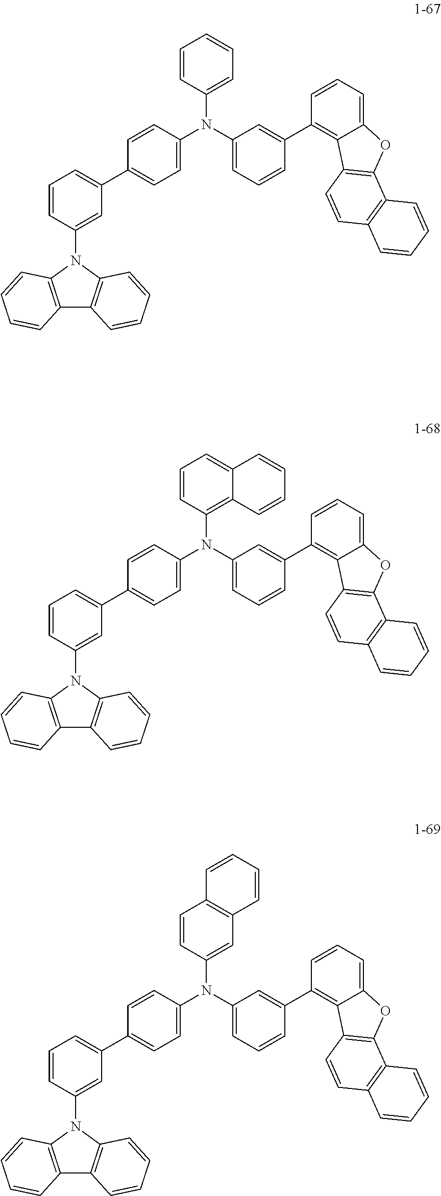 [SMILES: c1ccc(N(c2ccc(-c3cccc(-n4c5ccccc5c5ccccc54)c3)cc2)c2cccc(-c3cccc4oc5c6ccccc6ccc5c34)c2)cc1 ; c1cc(-c2cccc3oc4c5ccccc5ccc4c23)cc(N(c2ccc(-c3cccc(-n4c5ccccc5c5ccccc54)c3)cc2)c2cccc3ccccc23)c1 ; c1cc(-c2cccc3oc4c5ccccc5ccc4c23)cc(N(c2ccc(-c3cccc(-n4c5ccccc5c5ccccc54)c3)cc2)c2ccc3ccccc3c2)c1]

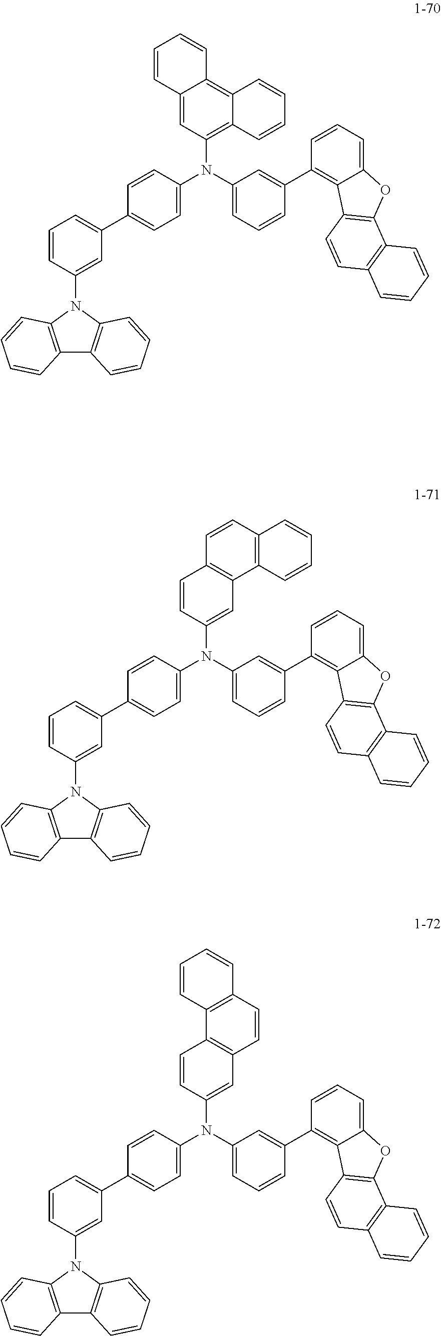 [SMILES: c1cc(-c2cccc3oc4c5ccccc5ccc4c23)cc(N(c2ccc(-c3cccc(-n4c5ccccc5c5ccccc54)c3)cc2)c2cc3ccccc3c3ccccc23)c1 ; c1cc(-c2cccc3oc4c5ccccc5ccc4c23)cc(N(c2ccc(-c3cccc(-n4c5ccccc5c5ccccc54)c3)cc2)c2ccc3ccc4ccccc4c3c2)c1 ; c1cc(-c2cccc3oc4c5ccccc5ccc4c23)cc(N(c2ccc(-c3cccc(-n4c5ccccc5c5ccccc54)c3)cc2)c2ccc3c(ccc4ccccc43)c2)c1]

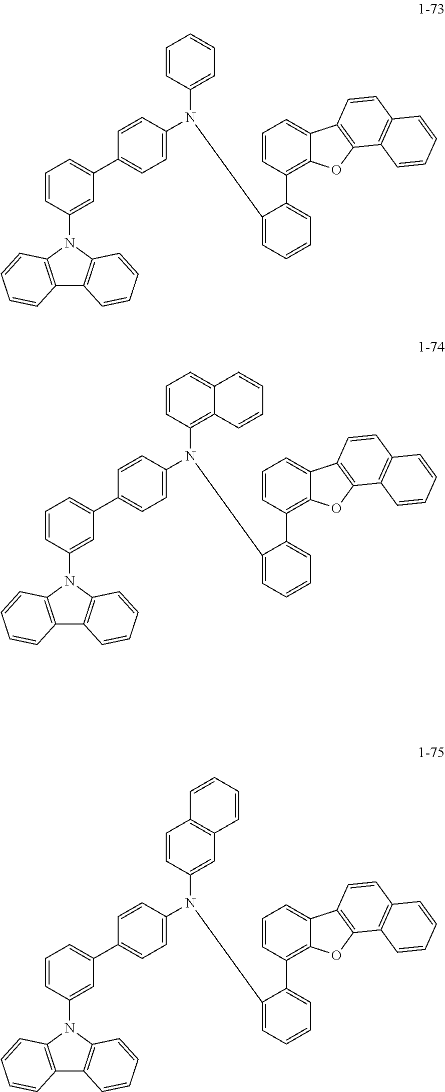 [SMILES: c1ccc(N(c2ccc(-c3cccc(-n4c5ccccc5c5ccccc54)c3)cc2)c2ccccc2-c2cccc3c2oc2c4ccccc4ccc32)cc1 ; c1cc(-c2ccc(N(c3ccccc3-c3cccc4c3oc3c5ccccc5ccc43)c3cccc4ccccc34)cc2)cc(-n2c3ccccc3c3ccccc32)c1 ; c1cc(-c2ccc(N(c3ccc4ccccc4c3)c3ccccc3-c3cccc4c3oc3c5ccccc5ccc43)cc2)cc(-n2c3ccccc3c3ccccc32)c1]

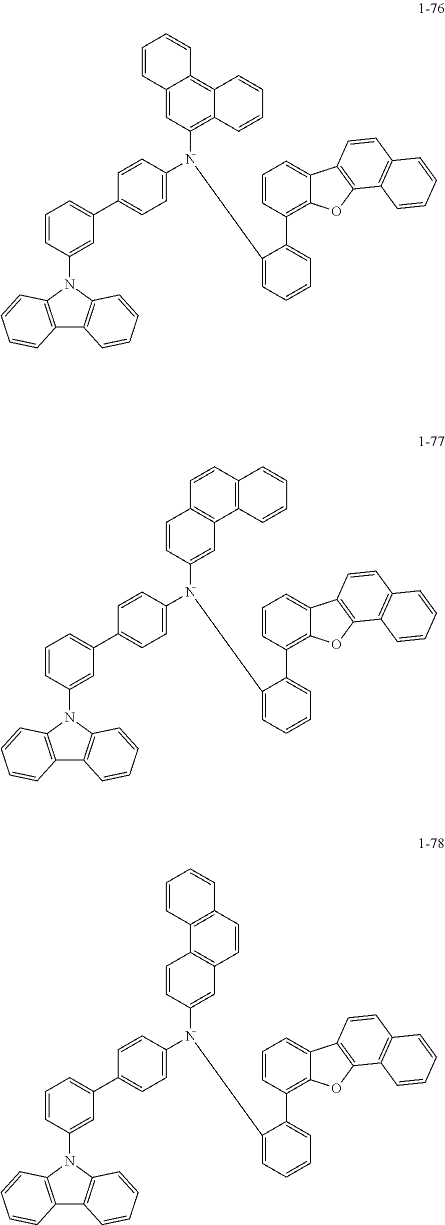 [SMILES: c1cc(-c2ccc(N(c3ccccc3-c3cccc4c3oc3c5ccccc5ccc43)c3cc4ccccc4c4ccccc34)cc2)cc(-n2c3ccccc3c3ccccc32)c1 ; c1cc(-c2ccc(N(c3ccc4ccc5ccccc5c4c3)c3ccccc3-c3cccc4c3oc3c5ccccc5ccc43)cc2)cc(-n2c3ccccc3c3ccccc32)c1 ; c1cc(-c2ccc(N(c3ccc4c(ccc5ccccc54)c3)c3ccccc3-c3cccc4c3oc3c5ccccc5ccc43)cc2)cc(-n2c3ccccc3c3ccccc32)c1]

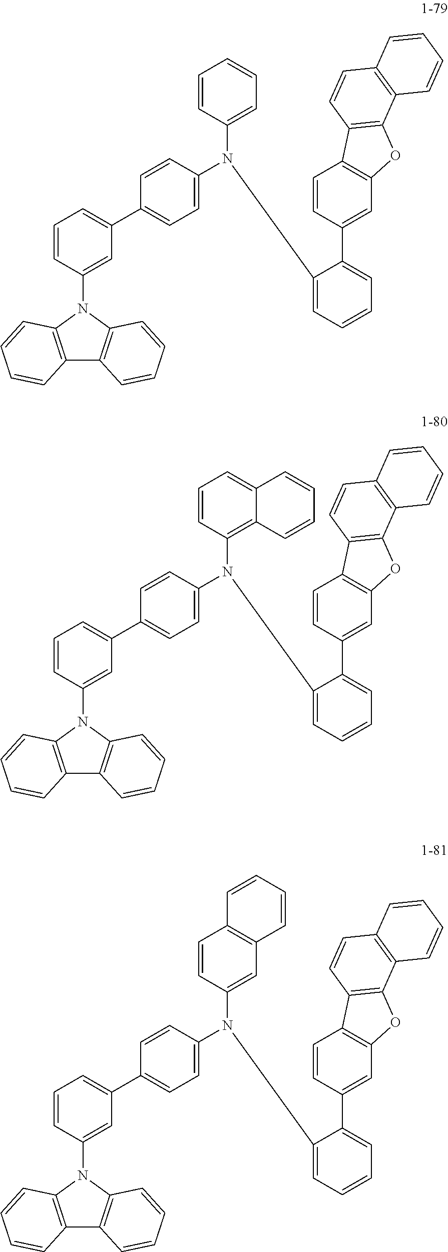 [SMILES: c1ccc(N(c2ccc(-c3cccc(-n4c5ccccc5c5ccccc54)c3)cc2)c2ccccc2-c2ccc3c(c2)oc2c4ccccc4ccc32)cc1 ; c1cc(-c2ccc(N(c3ccccc3-c3ccc4c(c3)oc3c5ccccc5ccc43)c3cccc4ccccc34)cc2)cc(-n2c3ccccc3c3ccccc32)c1 ; c1cc(-c2ccc(N(c3ccc4ccccc4c3)c3ccccc3-c3ccc4c(c3)oc3c5ccccc5ccc43)cc2)cc(-n2c3ccccc3c3ccccc32)c1]

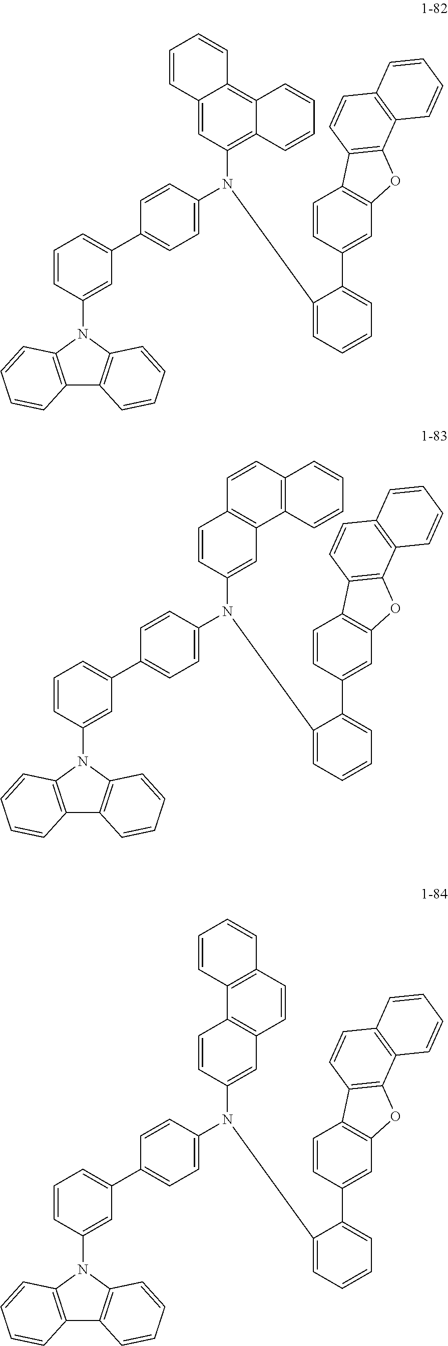 [SMILES: c1cc(-c2ccc(N(c3ccccc3-c3ccc4c(c3)oc3c5ccccc5ccc43)c3cc4ccccc4c4ccccc34)cc2)cc(-n2c3ccccc3c3ccccc32)c1 ; c1cc(-c2ccc(N(c3ccc4ccc5ccccc5c4c3)c3ccccc3-c3ccc4c(c3)oc3c5ccccc5ccc43)cc2)cc(-n2c3ccccc3c3ccccc32)c1 ; c1cc(-c2ccc(N(c3ccc4c(ccc5ccccc54)c3)c3ccccc3-c3ccc4c(c3)oc3c5ccccc5ccc43)cc2)cc(-n2c3ccccc3c3ccccc32)c1]

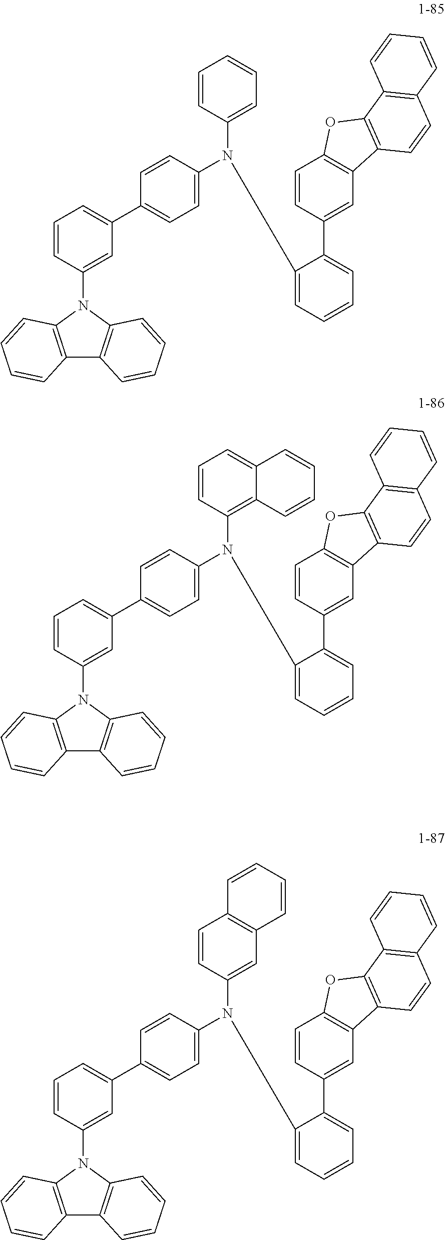 [SMILES: c1ccc(N(c2ccc(-c3cccc(-n4c5ccccc5c5ccccc54)c3)cc2)c2ccccc2-c2ccc3oc4c5ccccc5ccc4c3c2)cc1 ; c1cc(-c2ccc(N(c3ccccc3-c3ccc4oc5c6ccccc6ccc5c4c3)c3cccc4ccccc34)cc2)cc(-n2c3ccccc3c3ccccc32)c1 ; c1cc(-c2ccc(N(c3ccc4ccccc4c3)c3ccccc3-c3ccc4oc5c6ccccc6ccc5c4c3)cc2)cc(-n2c3ccccc3c3ccccc32)c1]

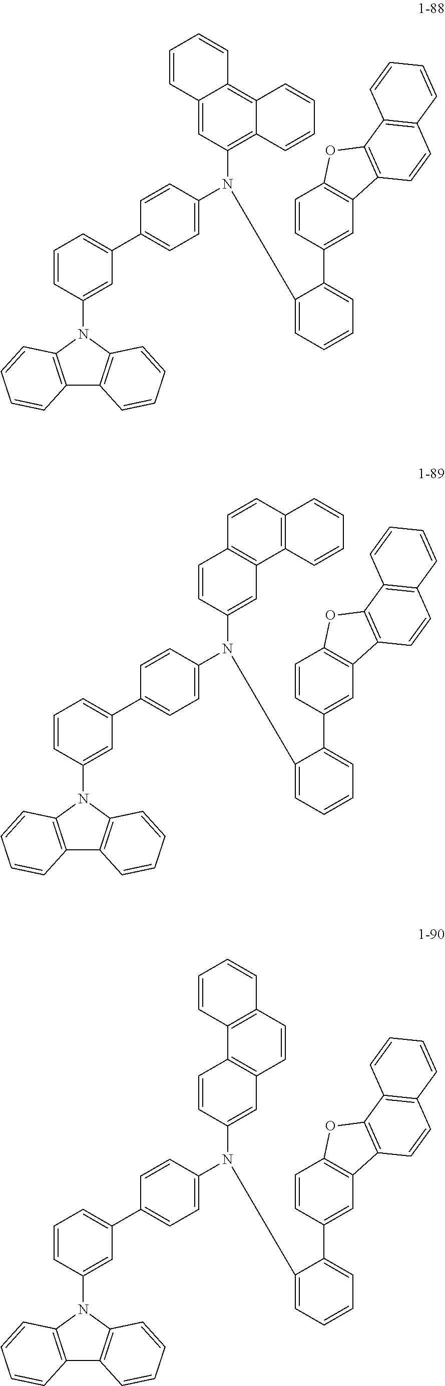 [SMILES: c1cc(-c2ccc(N(c3ccccc3-c3ccc4oc5c6ccccc6ccc5c4c3)c3cc4ccccc4c4ccccc34)cc2)cc(-n2c3ccccc3c3ccccc32)c1 ; c1cc(-c2ccc(N(c3ccc4ccc5ccccc5c4c3)c3ccccc3-c3ccc4oc5c6ccccc6ccc5c4c3)cc2)cc(-n2c3ccccc3c3ccccc32)c1 ; c1cc(-c2ccc(N(c3ccc4c(ccc5ccccc54)c3)c3ccccc3-c3ccc4oc5c6ccccc6ccc5c4c3)cc2)cc(-n2c3ccccc3c3ccccc32)c1]

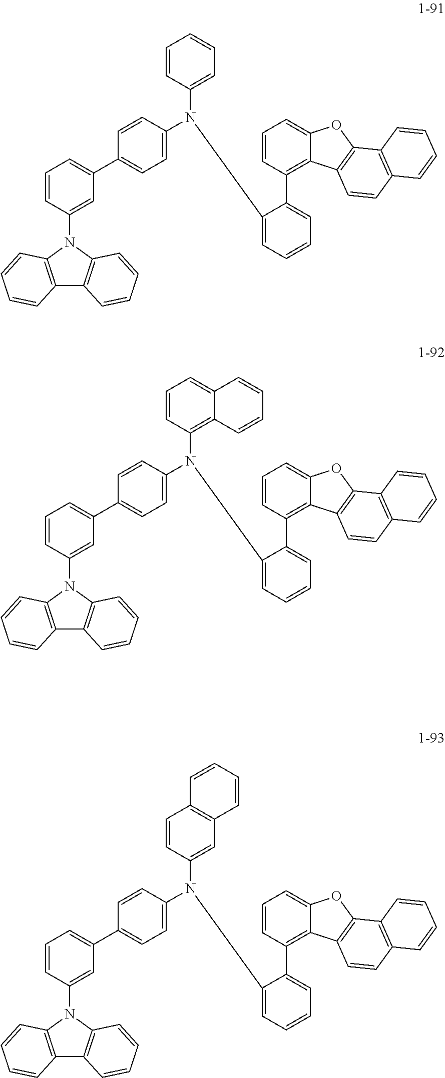 [SMILES: c1ccc(N(c2ccc(-c3cccc(-n4c5ccccc5c5ccccc54)c3)cc2)c2ccccc2-c2cccc3oc4c5ccccc5ccc4c23)cc1 ; c1cc(-c2ccc(N(c3ccccc3-c3cccc4oc5c6ccccc6ccc5c34)c3cccc4ccccc34)cc2)cc(-n2c3ccccc3c3ccccc32)c1 ; c1cc(-c2ccc(N(c3ccc4ccccc4c3)c3ccccc3-c3cccc4oc5c6ccccc6ccc5c34)cc2)cc(-n2c3ccccc3c3ccccc32)c1]

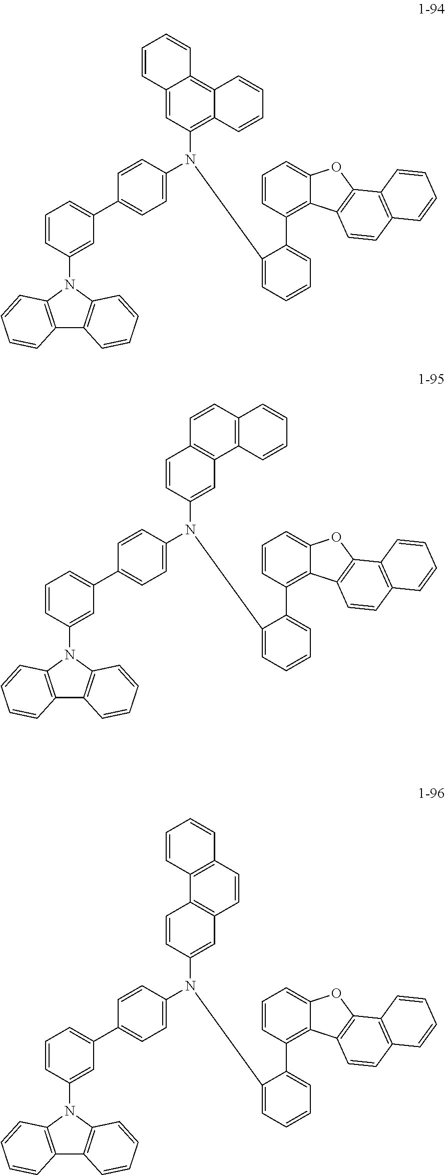 [SMILES: c1cc(-c2ccc(N(c3ccccc3-c3cccc4oc5c6ccccc6ccc5c34)c3cc4ccccc4c4ccccc34)cc2)cc(-n2c3ccccc3c3ccccc32)c1 ; c1cc(-c2ccc(N(c3ccc4ccc5ccccc5c4c3)c3ccccc3-c3cccc4oc5c6ccccc6ccc5c34)cc2)cc(-n2c3ccccc3c3ccccc32)c1 ; c1cc(-c2ccc(N(c3ccc4c(ccc5ccccc54)c3)c3ccccc3-c3cccc4oc5c6ccccc6ccc5c34)cc2)cc(-n2c3ccccc3c3ccccc32)c1]

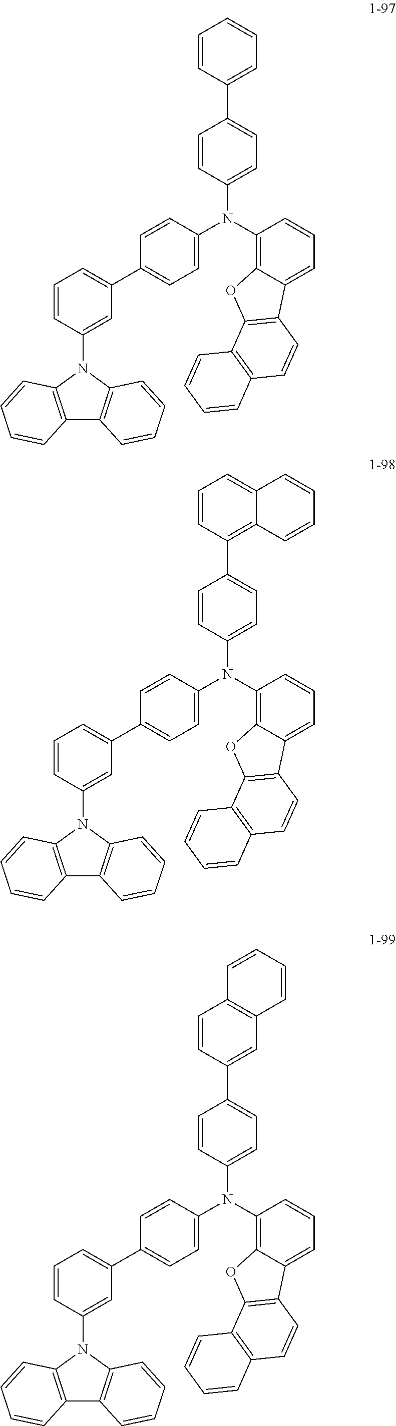 [SMILES: c1ccc(-c2ccc(N(c3ccc(-c4cccc(-n5c6ccccc6c6ccccc65)c4)cc3)c3cccc4c3oc3c5ccccc5ccc43)cc2)cc1 ; c1cc(-c2ccc(N(c3ccc(-c4cccc5ccccc45)cc3)c3cccc4c3oc3c5ccccc5ccc43)cc2)cc(-n2c3ccccc3c3ccccc32)c1 ; c1cc(-c2ccc(N(c3ccc(-c4ccc5ccccc5c4)cc3)c3cccc4c3oc3c5ccccc5ccc43)cc2)cc(-n2c3ccccc3c3ccccc32)c1]

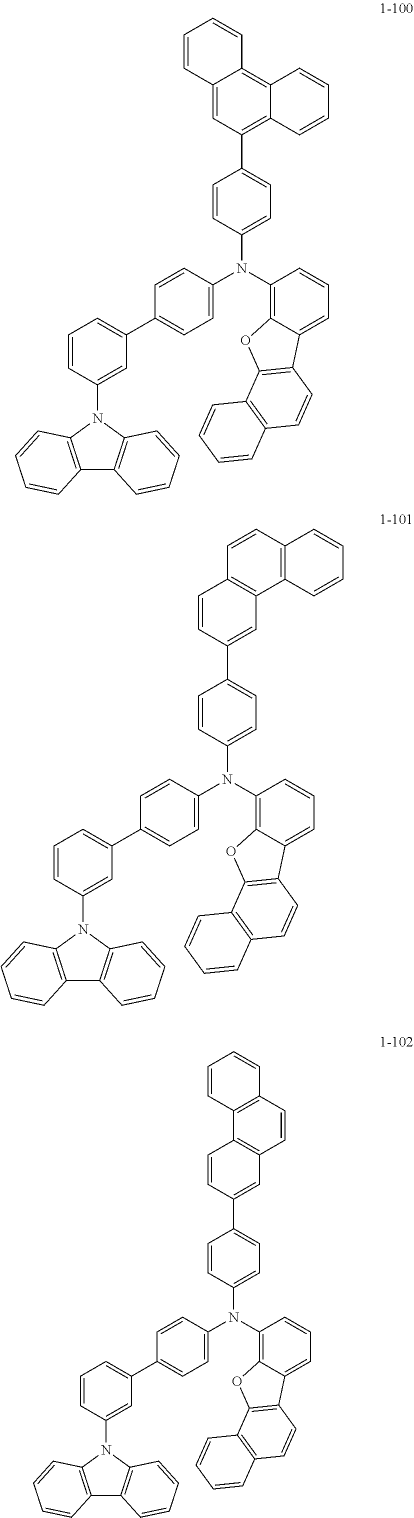 [SMILES: c1cc(-c2ccc(N(c3ccc(-c4cc5ccccc5c5ccccc45)cc3)c3cccc4c3oc3c5ccccc5ccc43)cc2)cc(-n2c3ccccc3c3ccccc32)c1 ; c1cc(-c2ccc(N(c3ccc(-c4ccc5ccc6ccccc6c5c4)cc3)c3cccc4c3oc3c5ccccc5ccc43)cc2)cc(-n2c3ccccc3c3ccccc32)c1 ; c1cc(-c2ccc(N(c3ccc(-c4ccc5c(ccc6ccccc65)c4)cc3)c3cccc4c3oc3c5ccccc5ccc43)cc2)cc(-n2c3ccccc3c3ccccc32)c1]

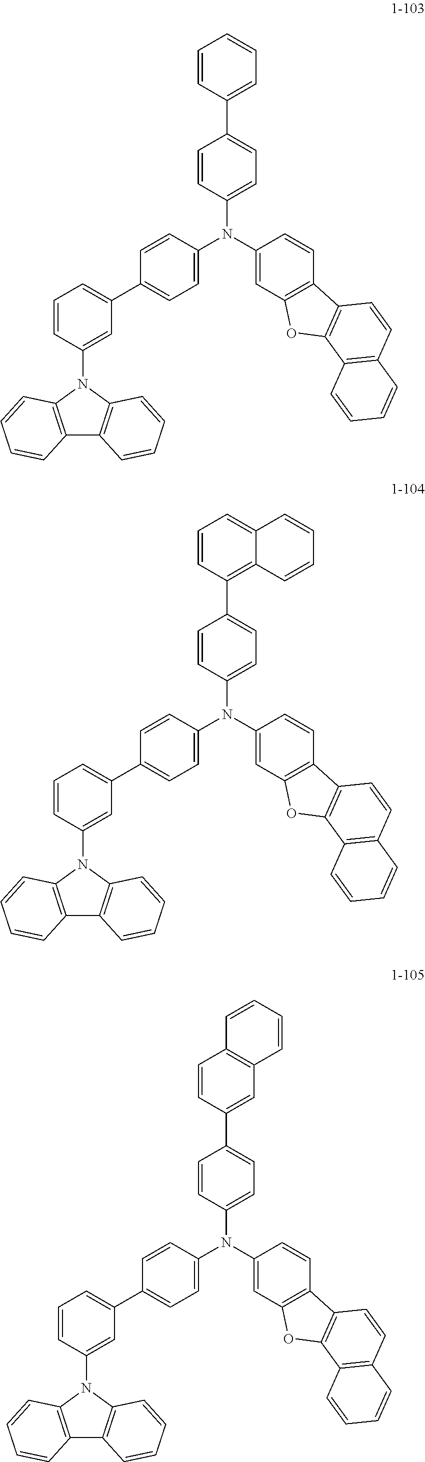 [SMILES: c1ccc(-c2ccc(N(c3ccc(-c4cccc(-n5c6ccccc6c6ccccc65)c4)cc3)c3ccc4c(c3)oc3c5ccccc5ccc43)cc2)cc1 ; c1cc(-c2ccc(N(c3ccc(-c4cccc5ccccc45)cc3)c3ccc4c(c3)oc3c5ccccc5ccc43)cc2)cc(-n2c3ccccc3c3ccccc32)c1 ; c1cc(-c2ccc(N(c3ccc(-c4ccc5ccccc5c4)cc3)c3ccc4c(c3)oc3c5ccccc5ccc43)cc2)cc(-n2c3ccccc3c3ccccc32)c1]

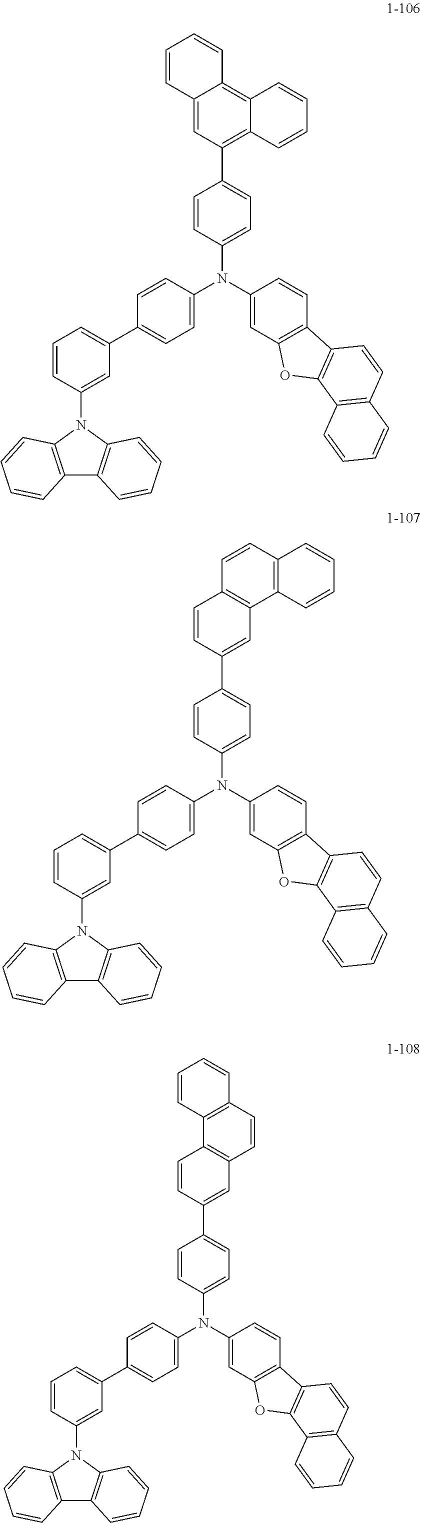 [SMILES: c1cc(-c2ccc(N(c3ccc(-c4cc5ccccc5c5ccccc45)cc3)c3ccc4c(c3)oc3c5ccccc5ccc43)cc2)cc(-n2c3ccccc3c3ccccc32)c1 ; c1cc(-c2ccc(N(c3ccc(-c4ccc5ccc6ccccc6c5c4)cc3)c3ccc4c(c3)oc3c5ccccc5ccc43)cc2)cc(-n2c3ccccc3c3ccccc32)c1 ; c1cc(-c2ccc(N(c3ccc(-c4ccc5c(ccc6ccccc65)c4)cc3)c3ccc4c(c3)oc3c5ccccc5ccc43)cc2)cc(-n2c3ccccc3c3ccccc32)c1]

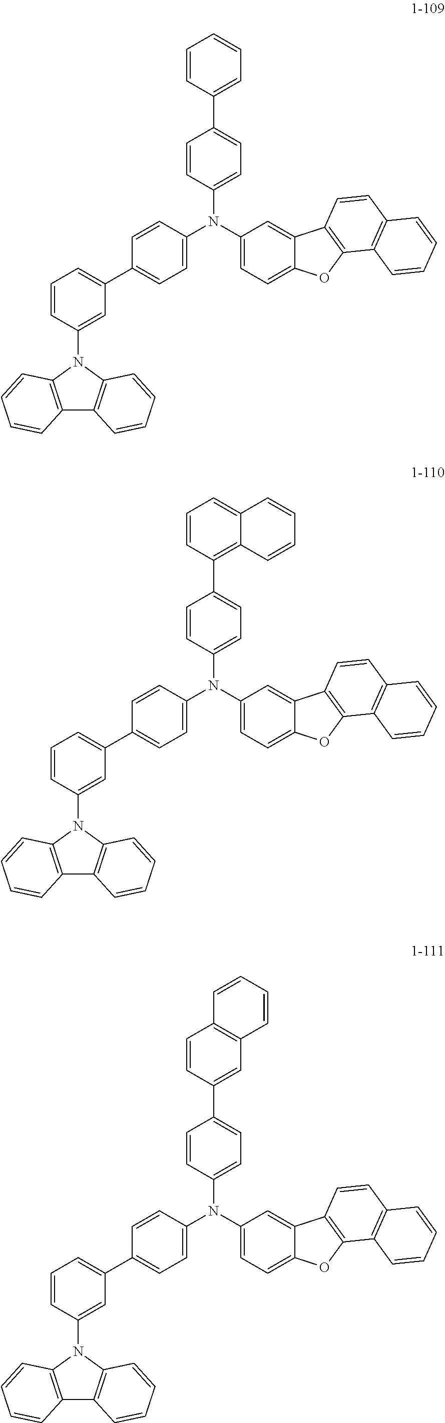 [SMILES: c1ccc(-c2ccc(N(c3ccc(-c4cccc(-n5c6ccccc6c6ccccc65)c4)cc3)c3ccc4oc5c6ccccc6ccc5c4c3)cc2)cc1 ; c1cc(-c2ccc(N(c3ccc(-c4cccc5ccccc45)cc3)c3ccc4oc5c6ccccc6ccc5c4c3)cc2)cc(-n2c3ccccc3c3ccccc32)c1 ; c1cc(-c2ccc(N(c3ccc(-c4ccc5ccccc5c4)cc3)c3ccc4oc5c6ccccc6ccc5c4c3)cc2)cc(-n2c3ccccc3c3ccccc32)c1]

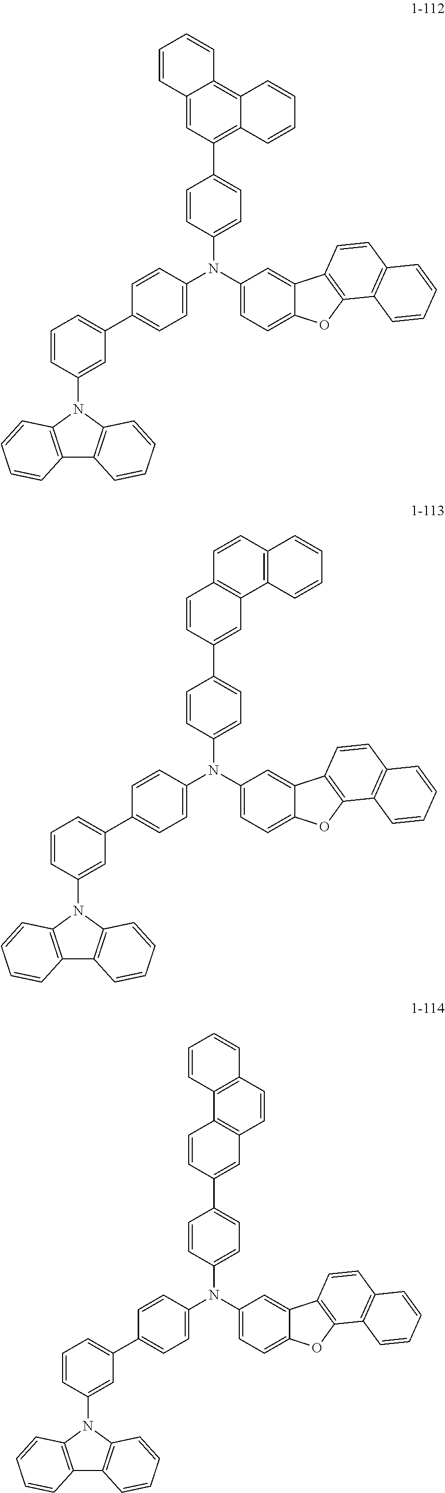 [SMILES: c1cc(-c2ccc(N(c3ccc(-c4cc5ccccc5c5ccccc45)cc3)c3ccc4oc5c6ccccc6ccc5c4c3)cc2)cc(-n2c3ccccc3c3ccccc32)c1 ; c1cc(-c2ccc(N(c3ccc(-c4ccc5ccc6ccccc6c5c4)cc3)c3ccc4oc5c6ccccc6ccc5c4c3)cc2)cc(-n2c3ccccc3c3ccccc32)c1 ; c1cc(-c2ccc(N(c3ccc(-c4ccc5c(ccc6ccccc65)c4)cc3)c3ccc4oc5c6ccccc6ccc5c4c3)cc2)cc(-n2c3ccccc3c3ccccc32)c1]

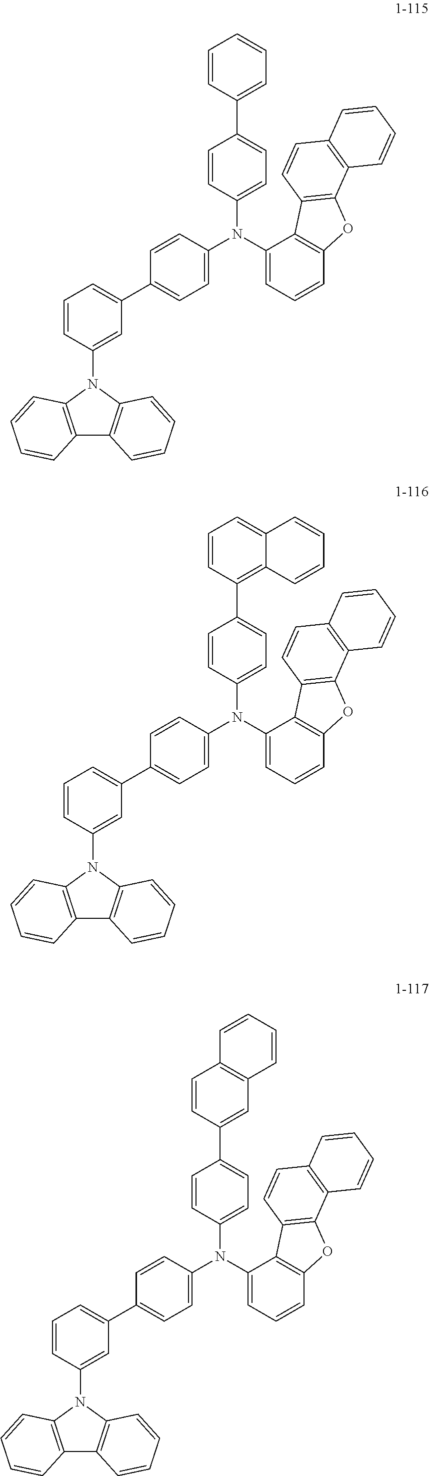 [SMILES: c1ccc(-c2ccc(N(c3ccc(-c4cccc(-n5c6ccccc6c6ccccc65)c4)cc3)c3cccc4oc5c6ccccc6ccc5c34)cc2)cc1 ; c1cc(-c2ccc(N(c3ccc(-c4cccc5ccccc45)cc3)c3cccc4oc5c6ccccc6ccc5c34)cc2)cc(-n2c3ccccc3c3ccccc32)c1 ; c1cc(-c2ccc(N(c3ccc(-c4ccc5ccccc5c4)cc3)c3cccc4oc5c6ccccc6ccc5c34)cc2)cc(-n2c3ccccc3c3ccccc32)c1]

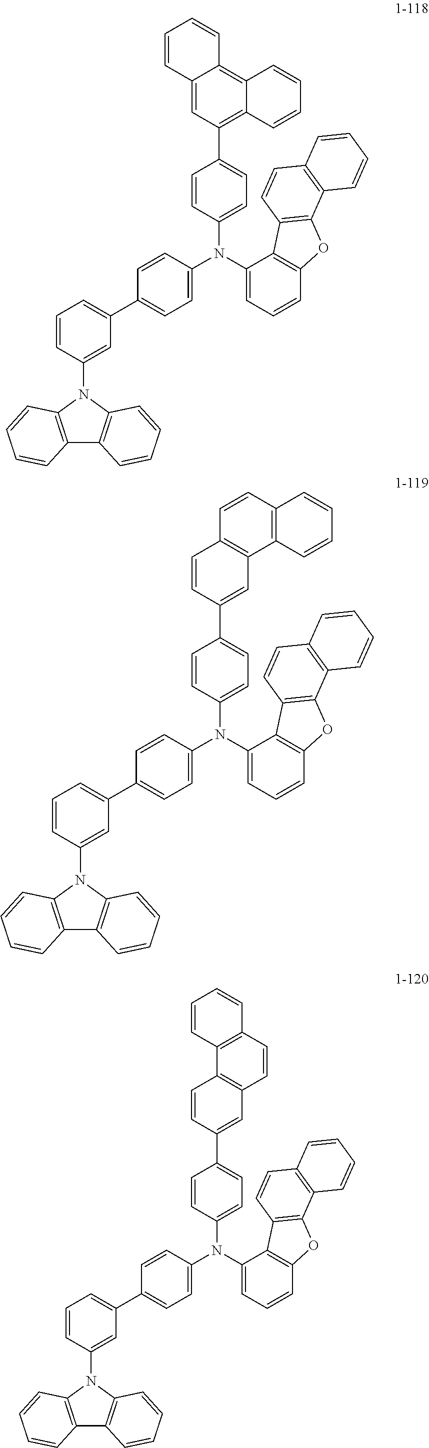 [SMILES: c1cc(-c2ccc(N(c3ccc(-c4cc5ccccc5c5ccccc45)cc3)c3cccc4oc5c6ccccc6ccc5c34)cc2)cc(-n2c3ccccc3c3ccccc32)c1 ; c1cc(-c2ccc(N(c3ccc(-c4ccc5ccc6ccccc6c5c4)cc3)c3cccc4oc5c6ccccc6ccc5c34)cc2)cc(-n2c3ccccc3c3ccccc32)c1 ; c1cc(-c2ccc(N(c3ccc(-c4ccc5c(ccc6ccccc65)c4)cc3)c3cccc4oc5c6ccccc6ccc5c34)cc2)cc(-n2c3ccccc3c3ccccc32)c1]

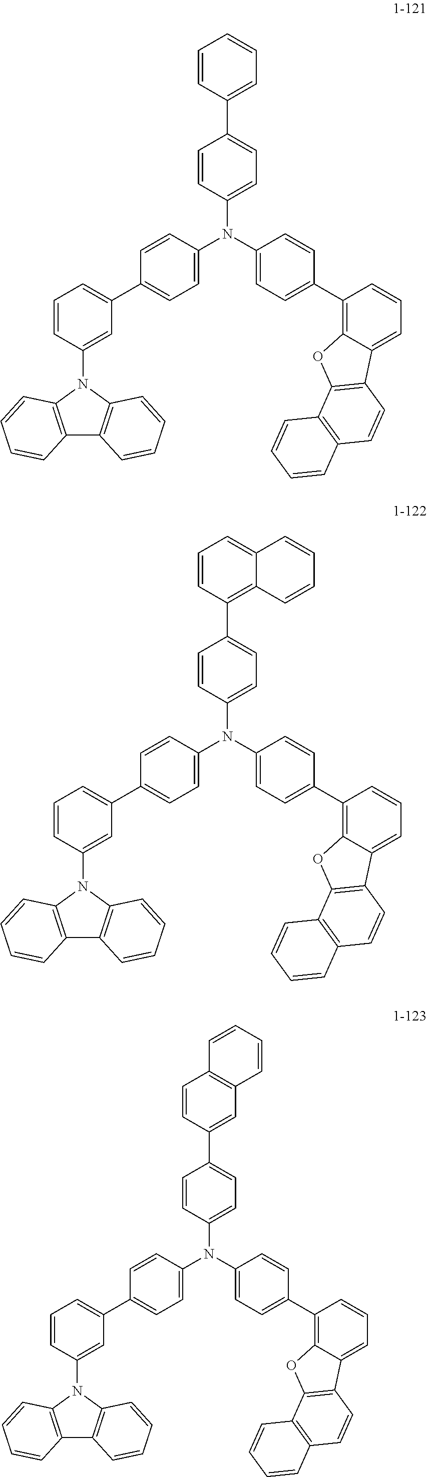 [SMILES: c1ccc(-c2ccc(N(c3ccc(-c4cccc(-n5c6ccccc6c6ccccc65)c4)cc3)c3ccc(-c4cccc5c4oc4c6ccccc6ccc54)cc3)cc2)cc1 ; c1cc(-c2ccc(N(c3ccc(-c4cccc5ccccc45)cc3)c3ccc(-c4cccc5c4oc4c6ccccc6ccc54)cc3)cc2)cc(-n2c3ccccc3c3ccccc32)c1 ; c1cc(-c2ccc(N(c3ccc(-c4ccc5ccccc5c4)cc3)c3ccc(-c4cccc5c4oc4c6ccccc6ccc54)cc3)cc2)cc(-n2c3ccccc3c3ccccc32)c1]

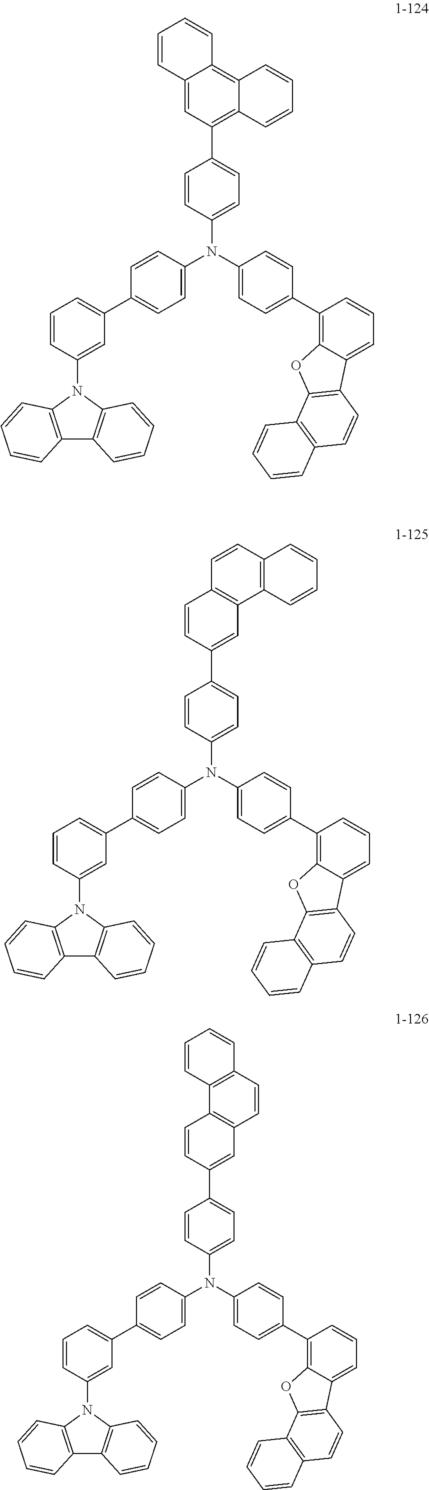 [SMILES: c1cc(-c2ccc(N(c3ccc(-c4cc5ccccc5c5ccccc45)cc3)c3ccc(-c4cccc5c4oc4c6ccccc6ccc54)cc3)cc2)cc(-n2c3ccccc3c3ccccc32)c1 ; c1cc(-c2ccc(N(c3ccc(-c4ccc5ccc6ccccc6c5c4)cc3)c3ccc(-c4cccc5c4oc4c6ccccc6ccc54)cc3)cc2)cc(-n2c3ccccc3c3ccccc32)c1 ; c1cc(-c2ccc(N(c3ccc(-c4ccc5c(ccc6ccccc65)c4)cc3)c3ccc(-c4cccc5c4oc4c6ccccc6ccc54)cc3)cc2)cc(-n2c3ccccc3c3ccccc32)c1]

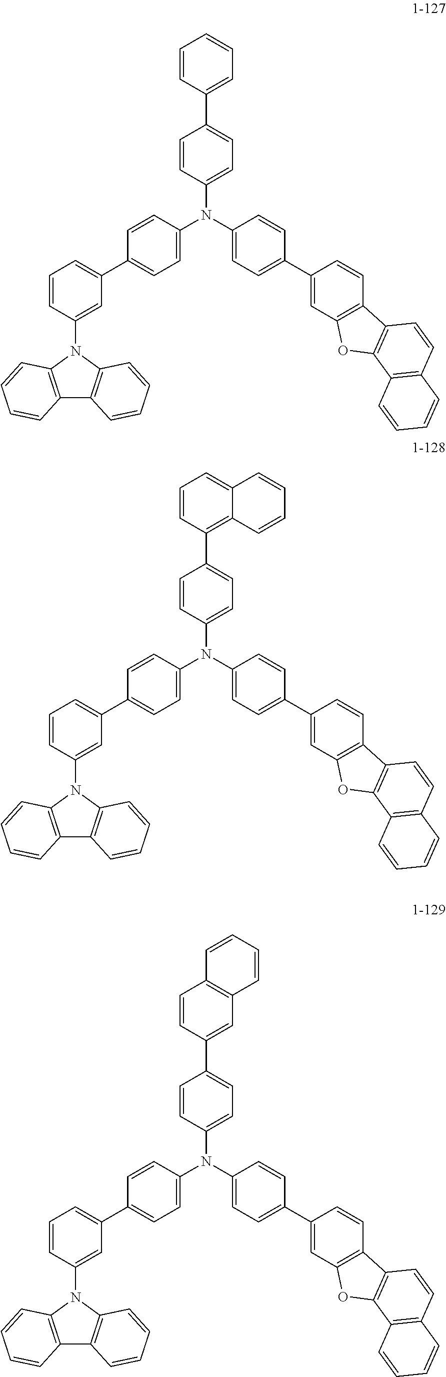 [SMILES: c1ccc(-c2ccc(N(c3ccc(-c4cccc(-n5c6ccccc6c6ccccc65)c4)cc3)c3ccc(-c4ccc5c(c4)oc4c6ccccc6ccc54)cc3)cc2)cc1 ; c1cc(-c2ccc(N(c3ccc(-c4ccc5c(c4)oc4c6ccccc6ccc54)cc3)c3ccc(-c4cccc5ccccc45)cc3)cc2)cc(-n2c3ccccc3c3ccccc32)c1 ; c1cc(-c2ccc(N(c3ccc(-c4ccc5ccccc5c4)cc3)c3ccc(-c4ccc5c(c4)oc4c6ccccc6ccc54)cc3)cc2)cc(-n2c3ccccc3c3ccccc32)c1]

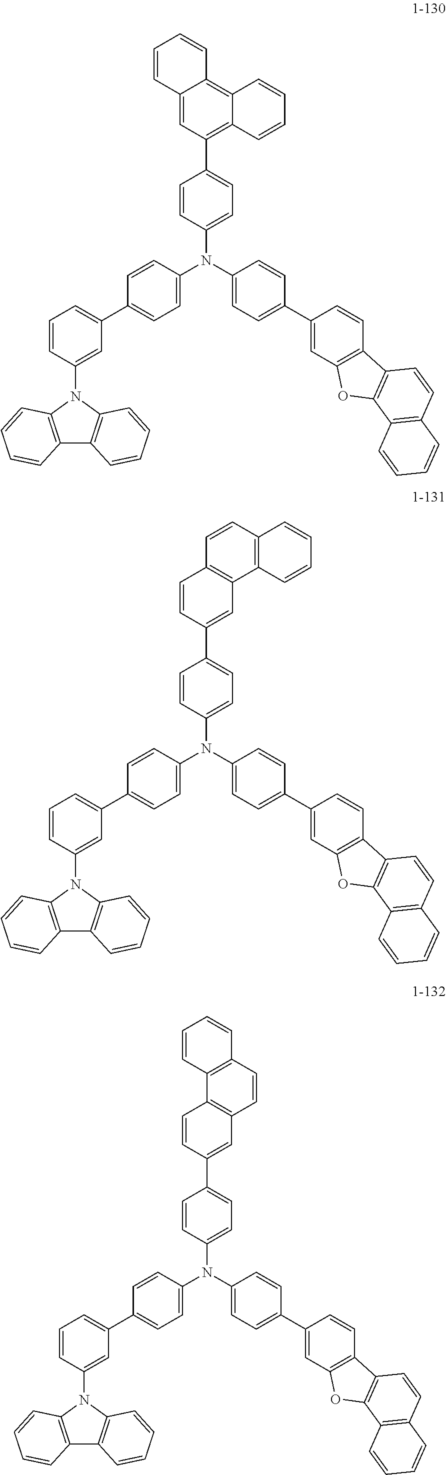 [SMILES: c1cc(-c2ccc(N(c3ccc(-c4ccc5c(c4)oc4c6ccccc6ccc54)cc3)c3ccc(-c4cc5ccccc5c5ccccc45)cc3)cc2)cc(-n2c3ccccc3c3ccccc32)c1 ; c1cc(-c2ccc(N(c3ccc(-c4ccc5c(c4)oc4c6ccccc6ccc54)cc3)c3ccc(-c4ccc5ccc6ccccc6c5c4)cc3)cc2)cc(-n2c3ccccc3c3ccccc32)c1 ; c1cc(-c2ccc(N(c3ccc(-c4ccc5c(ccc6ccccc65)c4)cc3)c3ccc(-c4ccc5c(c4)oc4c6ccccc6ccc54)cc3)cc2)cc(-n2c3ccccc3c3ccccc32)c1]

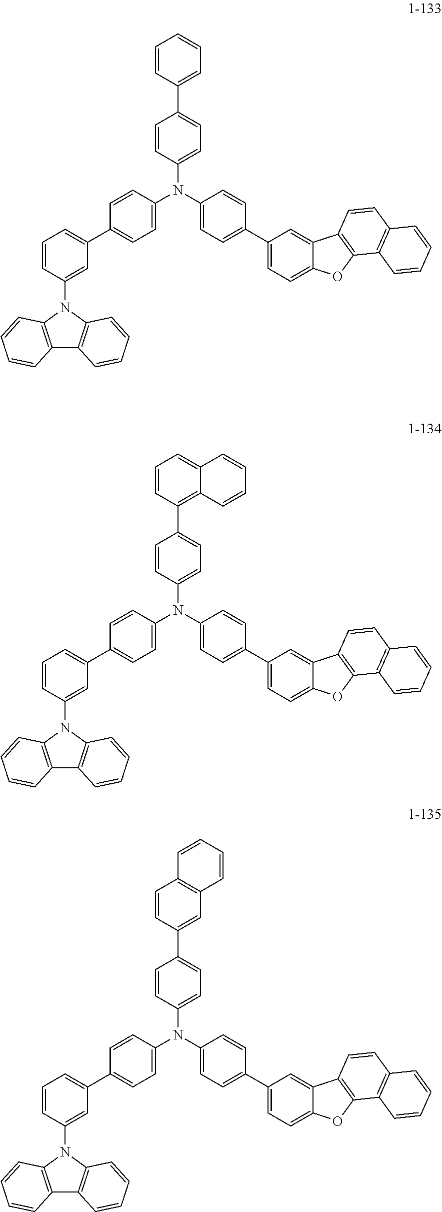 [SMILES: c1ccc(-c2ccc(N(c3ccc(-c4cccc(-n5c6ccccc6c6ccccc65)c4)cc3)c3ccc(-c4ccc5oc6c7ccccc7ccc6c5c4)cc3)cc2)cc1 ; c1cc(-c2ccc(N(c3ccc(-c4ccc5oc6c7ccccc7ccc6c5c4)cc3)c3ccc(-c4cccc5ccccc45)cc3)cc2)cc(-n2c3ccccc3c3ccccc32)c1 ; c1cc(-c2ccc(N(c3ccc(-c4ccc5ccccc5c4)cc3)c3ccc(-c4ccc5oc6c7ccccc7ccc6c5c4)cc3)cc2)cc(-n2c3ccccc3c3ccccc32)c1]

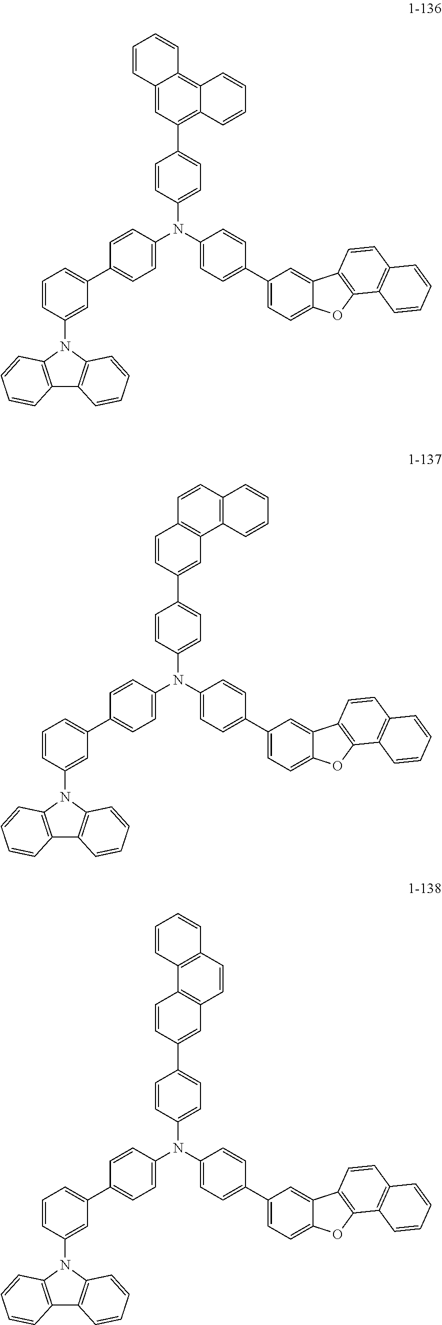 [SMILES: c1cc(-c2ccc(N(c3ccc(-c4ccc5oc6c7ccccc7ccc6c5c4)cc3)c3ccc(-c4cc5ccccc5c5ccccc45)cc3)cc2)cc(-n2c3ccccc3c3ccccc32)c1 ; c1cc(-c2ccc(N(c3ccc(-c4ccc5ccc6ccccc6c5c4)cc3)c3ccc(-c4ccc5oc6c7ccccc7ccc6c5c4)cc3)cc2)cc(-n2c3ccccc3c3ccccc32)c1 ; c1cc(-c2ccc(N(c3ccc(-c4ccc5c(ccc6ccccc65)c4)cc3)c3ccc(-c4ccc5oc6c7ccccc7ccc6c5c4)cc3)cc2)cc(-n2c3ccccc3c3ccccc32)c1]

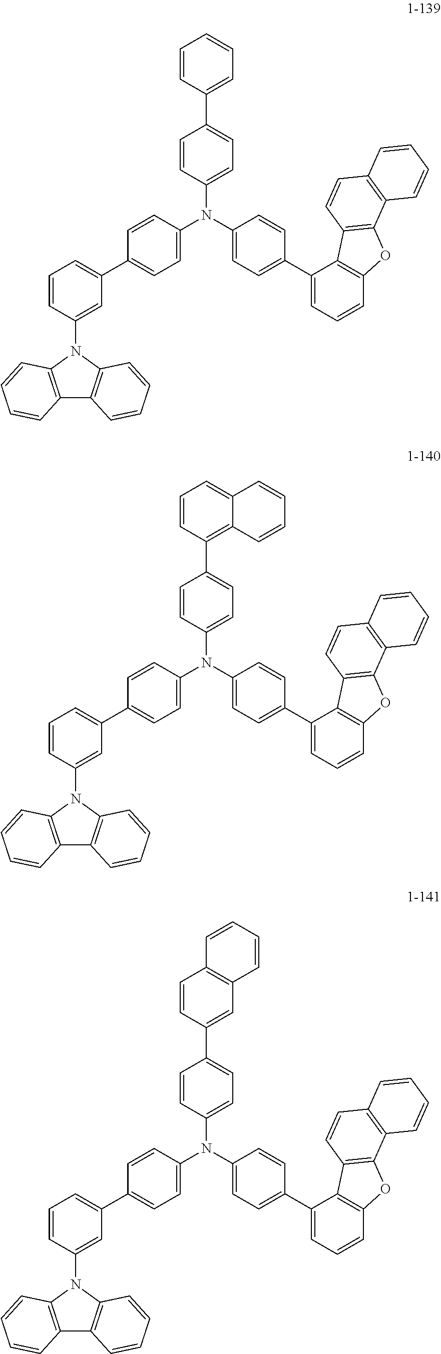 [SMILES: c1ccc(-c2ccc(N(c3ccc(-c4cccc(-n5c6ccccc6c6ccccc65)c4)cc3)c3ccc(-c4cccc5oc6c7ccccc7ccc6c45)cc3)cc2)cc1 ; c1cc(-c2ccc(N(c3ccc(-c4cccc5ccccc45)cc3)c3ccc(-c4cccc5oc6c7ccccc7ccc6c45)cc3)cc2)cc(-n2c3ccccc3c3ccccc32)c1 ; c1cc(-c2ccc(N(c3ccc(-c4ccc5ccccc5c4)cc3)c3ccc(-c4cccc5oc6c7ccccc7ccc6c45)cc3)cc2)cc(-n2c3ccccc3c3ccccc32)c1]

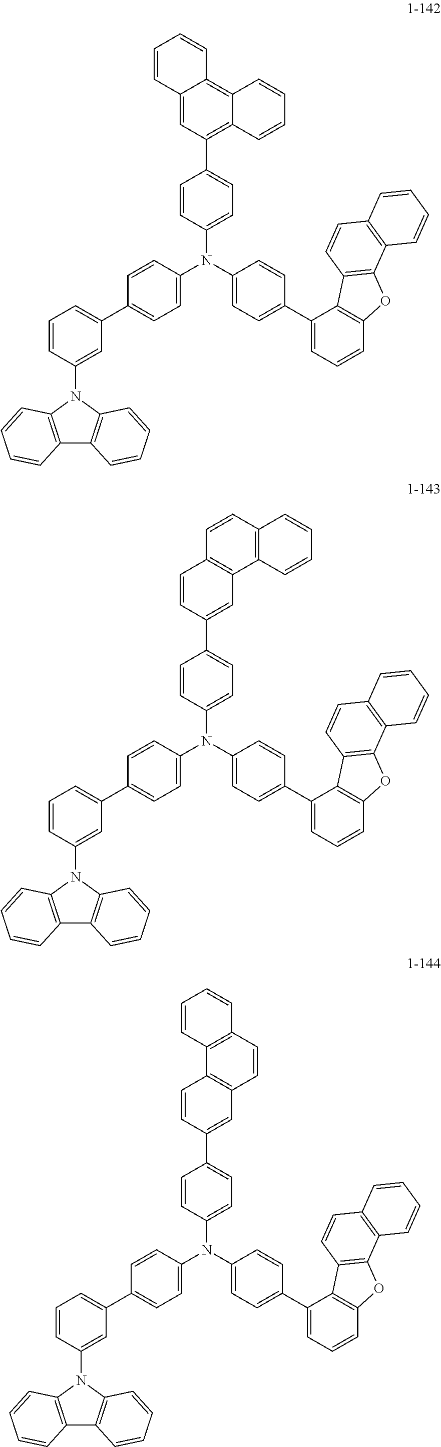 [SMILES: c1cc(-c2ccc(N(c3ccc(-c4cc5ccccc5c5ccccc45)cc3)c3ccc(-c4cccc5oc6c7ccccc7ccc6c45)cc3)cc2)cc(-n2c3ccccc3c3ccccc32)c1 ; c1cc(-c2ccc(N(c3ccc(-c4ccc5ccc6ccccc6c5c4)cc3)c3ccc(-c4cccc5oc6c7ccccc7ccc6c45)cc3)cc2)cc(-n2c3ccccc3c3ccccc32)c1 ; c1cc(-c2ccc(N(c3ccc(-c4ccc5c(ccc6ccccc65)c4)cc3)c3ccc(-c4cccc5oc6c7ccccc7ccc6c45)cc3)cc2)cc(-n2c3ccccc3c3ccccc32)c1]

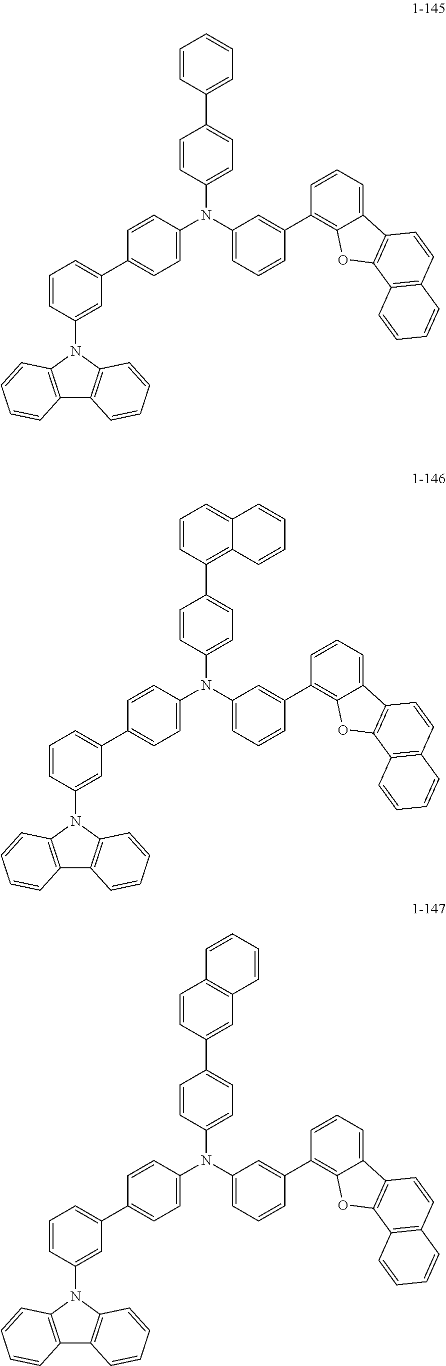 [SMILES: c1ccc(-c2ccc(N(c3ccc(-c4cccc(-n5c6ccccc6c6ccccc65)c4)cc3)c3cccc(-c4cccc5c4oc4c6ccccc6ccc54)c3)cc2)cc1 ; c1cc(-c2cccc3c2oc2c4ccccc4ccc32)cc(N(c2ccc(-c3cccc(-n4c5ccccc5c5ccccc54)c3)cc2)c2ccc(-c3cccc4ccccc34)cc2)c1 ; c1cc(-c2cccc3c2oc2c4ccccc4ccc32)cc(N(c2ccc(-c3cccc(-n4c5ccccc5c5ccccc54)c3)cc2)c2ccc(-c3ccc4ccccc4c3)cc2)c1]

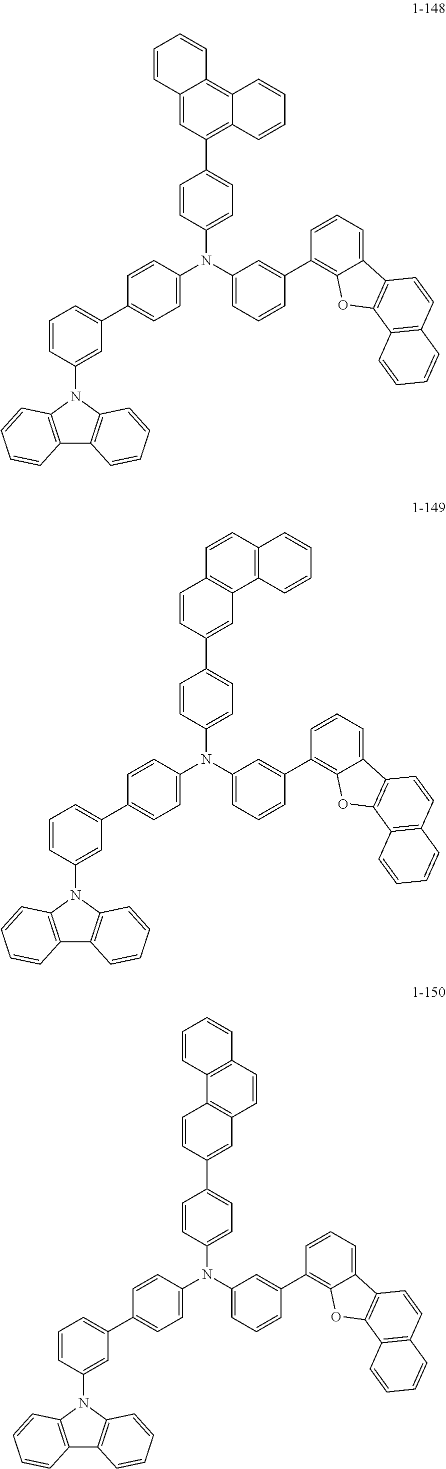 [SMILES: c1cc(-c2cccc3c2oc2c4ccccc4ccc32)cc(N(c2ccc(-c3cccc(-n4c5ccccc5c5ccccc54)c3)cc2)c2ccc(-c3cc4ccccc4c4ccccc34)cc2)c1 ; c1cc(-c2cccc3c2oc2c4ccccc4ccc32)cc(N(c2ccc(-c3cccc(-n4c5ccccc5c5ccccc54)c3)cc2)c2ccc(-c3ccc4ccc5ccccc5c4c3)cc2)c1 ; c1cc(-c2cccc3c2oc2c4ccccc4ccc32)cc(N(c2ccc(-c3cccc(-n4c5ccccc5c5ccccc54)c3)cc2)c2ccc(-c3ccc4c(ccc5ccccc54)c3)cc2)c1]

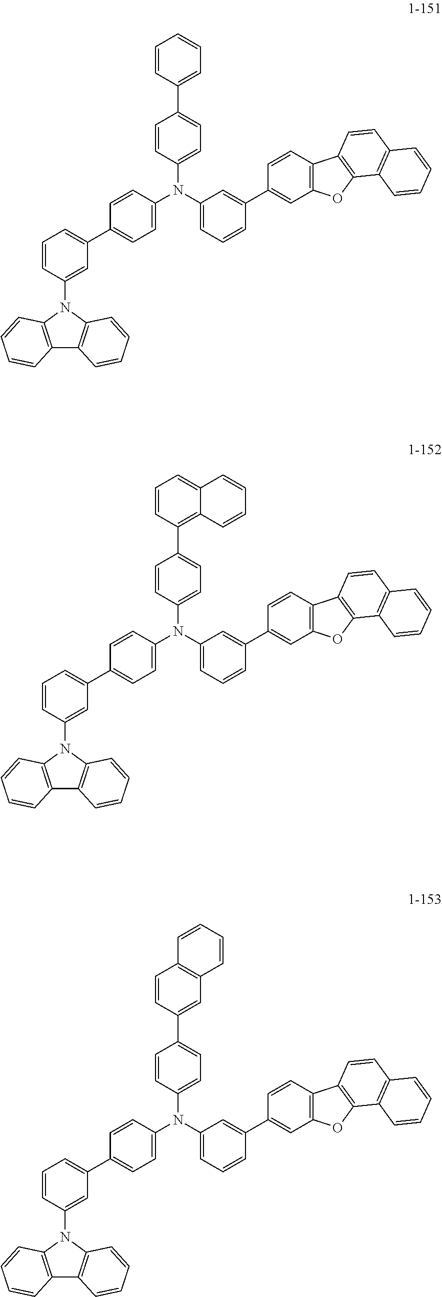 [SMILES: c1ccc(-c2ccc(N(c3ccc(-c4cccc(-n5c6ccccc6c6ccccc65)c4)cc3)c3cccc(-c4ccc5c(c4)oc4c6ccccc6ccc54)c3)cc2)cc1 ; c1cc(-c2ccc3c(c2)oc2c4ccccc4ccc32)cc(N(c2ccc(-c3cccc(-n4c5ccccc5c5ccccc54)c3)cc2)c2ccc(-c3cccc4ccccc34)cc2)c1 ; c1cc(-c2ccc3c(c2)oc2c4ccccc4ccc32)cc(N(c2ccc(-c3cccc(-n4c5ccccc5c5ccccc54)c3)cc2)c2ccc(-c3ccc4ccccc4c3)cc2)c1]

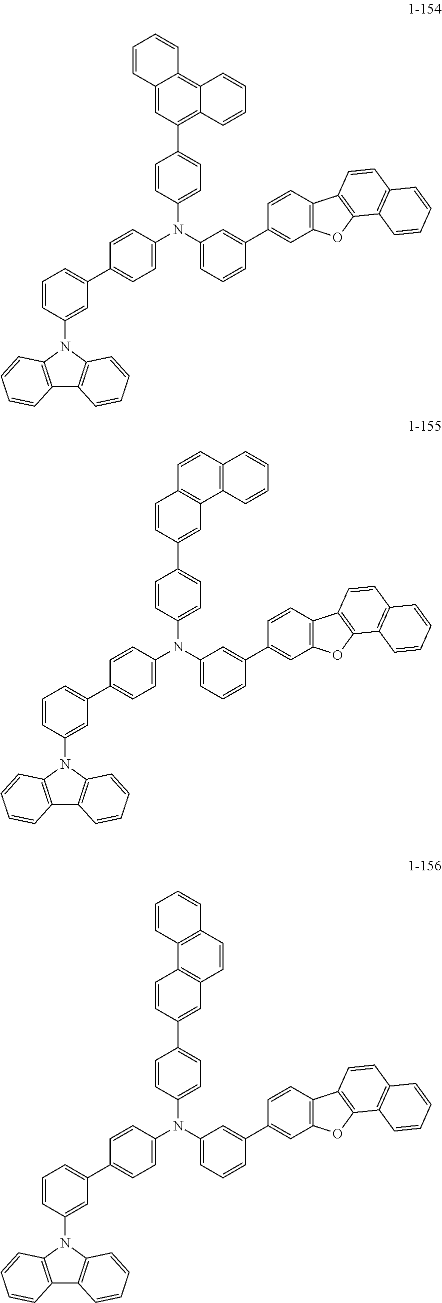 [SMILES: c1cc(-c2ccc3c(c2)oc2c4ccccc4ccc32)cc(N(c2ccc(-c3cccc(-n4c5ccccc5c5ccccc54)c3)cc2)c2ccc(-c3cc4ccccc4c4ccccc34)cc2)c1 ; c1cc(-c2ccc3c(c2)oc2c4ccccc4ccc32)cc(N(c2ccc(-c3cccc(-n4c5ccccc5c5ccccc54)c3)cc2)c2ccc(-c3ccc4ccc5ccccc5c4c3)cc2)c1 ; c1cc(-c2ccc3c(c2)oc2c4ccccc4ccc32)cc(N(c2ccc(-c3cccc(-n4c5ccccc5c5ccccc54)c3)cc2)c2ccc(-c3ccc4c(ccc5ccccc54)c3)cc2)c1]

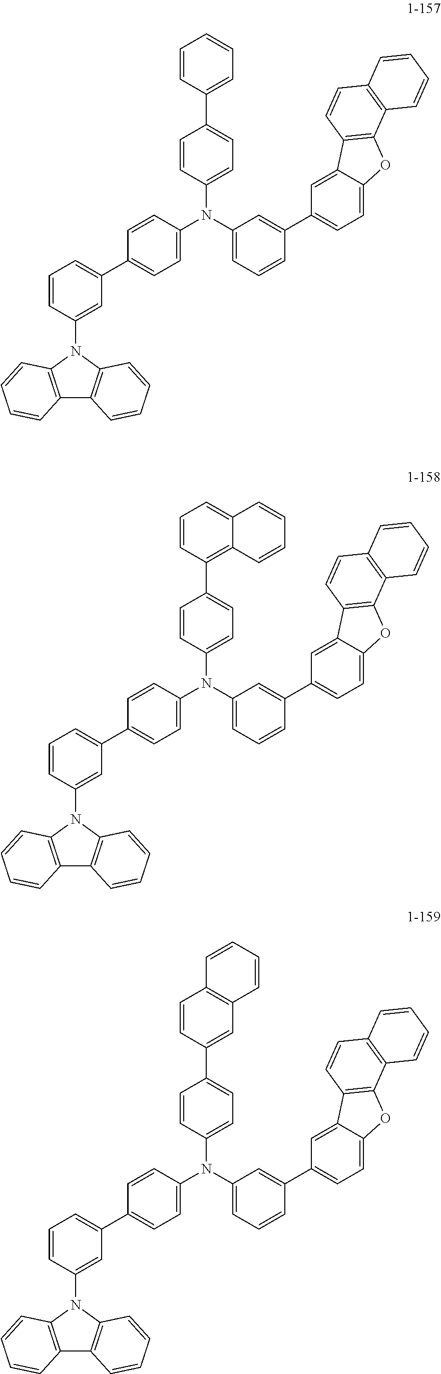 [SMILES: c1ccc(-c2ccc(N(c3ccc(-c4cccc(-n5c6ccccc6c6ccccc65)c4)cc3)c3cccc(-c4ccc5oc6c7ccccc7ccc6c5c4)c3)cc2)cc1 ; c1cc(-c2ccc3oc4c5ccccc5ccc4c3c2)cc(N(c2ccc(-c3cccc(-n4c5ccccc5c5ccccc54)c3)cc2)c2ccc(-c3cccc4ccccc34)cc2)c1 ; c1cc(-c2ccc3oc4c5ccccc5ccc4c3c2)cc(N(c2ccc(-c3cccc(-n4c5ccccc5c5ccccc54)c3)cc2)c2ccc(-c3ccc4ccccc4c3)cc2)c1]

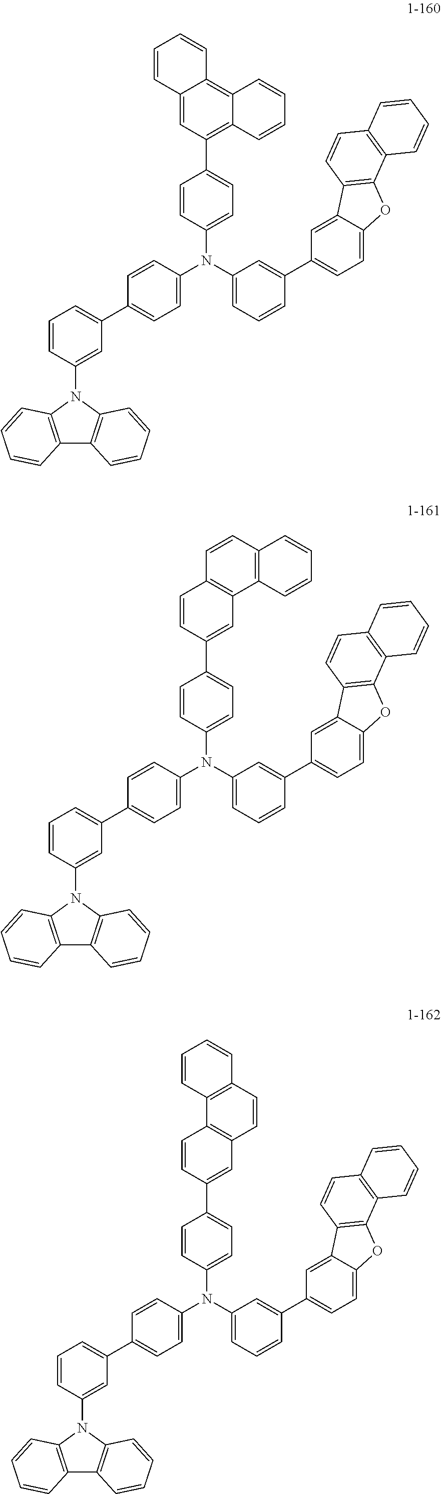 [SMILES: c1cc(-c2ccc3oc4c5ccccc5ccc4c3c2)cc(N(c2ccc(-c3cccc(-n4c5ccccc5c5ccccc54)c3)cc2)c2ccc(-c3cc4ccccc4c4ccccc34)cc2)c1 ; c1cc(-c2ccc3oc4c5ccccc5ccc4c3c2)cc(N(c2ccc(-c3cccc(-n4c5ccccc5c5ccccc54)c3)cc2)c2ccc(-c3ccc4ccc5ccccc5c4c3)cc2)c1 ; c1cc(-c2ccc3oc4c5ccccc5ccc4c3c2)cc(N(c2ccc(-c3cccc(-n4c5ccccc5c5ccccc54)c3)cc2)c2ccc(-c3ccc4c(ccc5ccccc54)c3)cc2)c1]

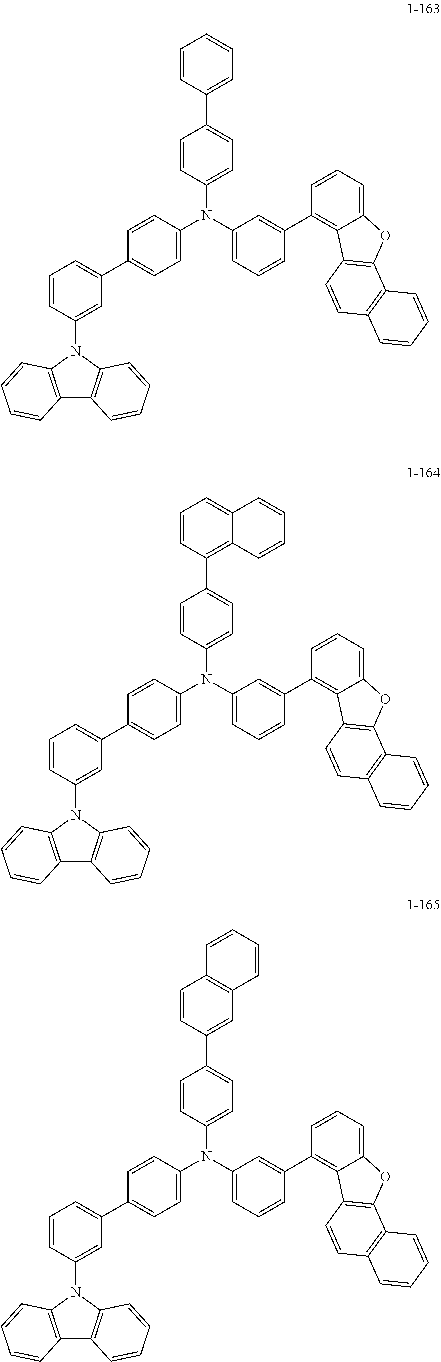 [SMILES: c1ccc(-c2ccc(N(c3ccc(-c4cccc(-n5c6ccccc6c6ccccc65)c4)cc3)c3cccc(-c4cccc5oc6c7ccccc7ccc6c45)c3)cc2)cc1 ; c1cc(-c2cccc3oc4c5ccccc5ccc4c23)cc(N(c2ccc(-c3cccc(-n4c5ccccc5c5ccccc54)c3)cc2)c2ccc(-c3cccc4ccccc34)cc2)c1 ; c1cc(-c2cccc3oc4c5ccccc5ccc4c23)cc(N(c2ccc(-c3cccc(-n4c5ccccc5c5ccccc54)c3)cc2)c2ccc(-c3ccc4ccccc4c3)cc2)c1]

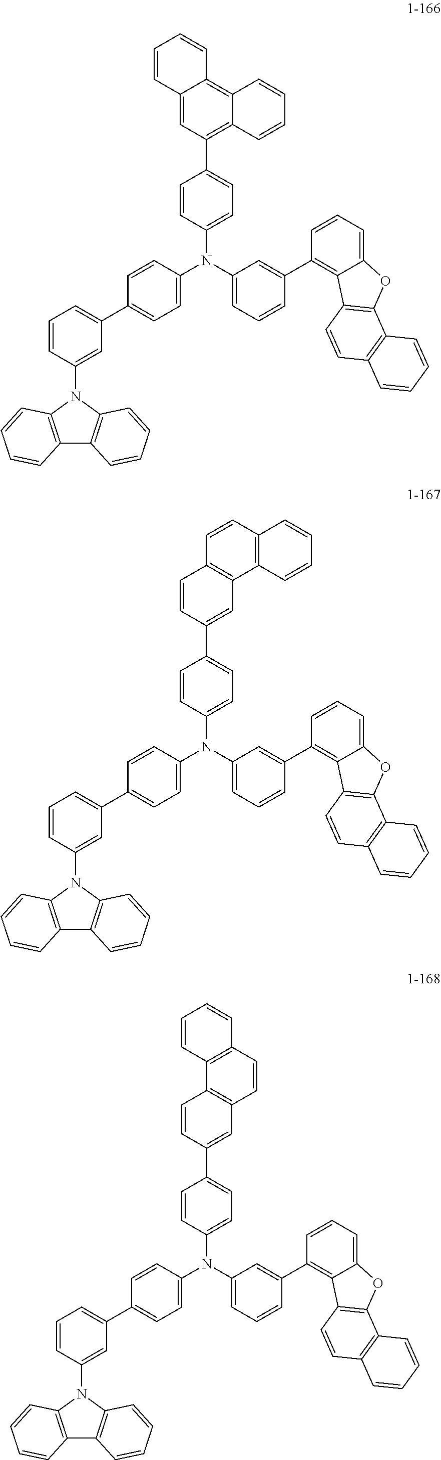 [SMILES: c1cc(-c2cccc3oc4c5ccccc5ccc4c23)cc(N(c2ccc(-c3cccc(-n4c5ccccc5c5ccccc54)c3)cc2)c2ccc(-c3cc4ccccc4c4ccccc34)cc2)c1 ; c1cc(-c2cccc3oc4c5ccccc5ccc4c23)cc(N(c2ccc(-c3cccc(-n4c5ccccc5c5ccccc54)c3)cc2)c2ccc(-c3ccc4ccc5ccccc5c4c3)cc2)c1 ; c1cc(-c2cccc3oc4c5ccccc5ccc4c23)cc(N(c2ccc(-c3cccc(-n4c5ccccc5c5ccccc54)c3)cc2)c2ccc(-c3ccc4c(ccc5ccccc54)c3)cc2)c1]

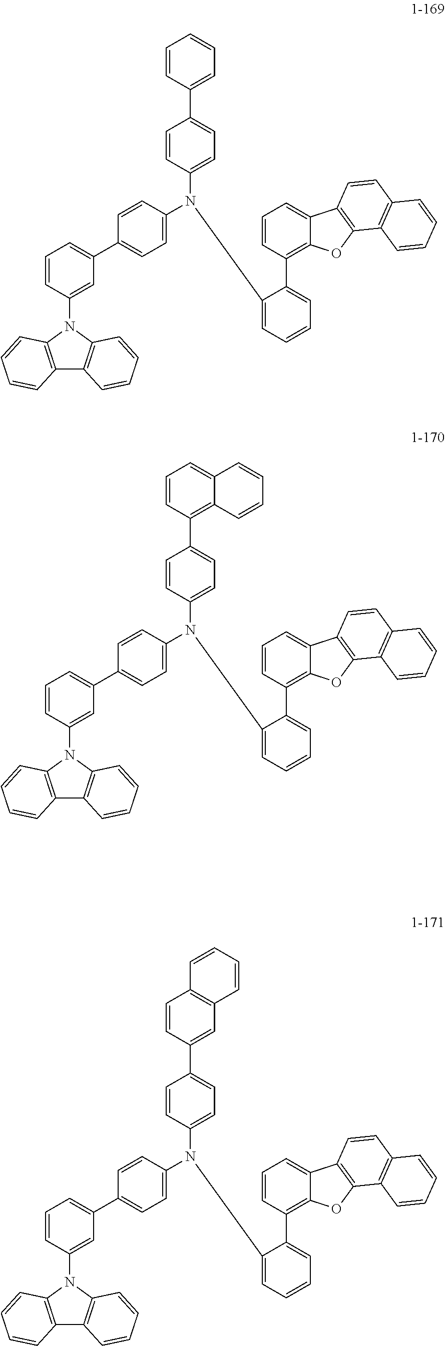 [SMILES: c1ccc(-c2ccc(N(c3ccc(-c4cccc(-n5c6ccccc6c6ccccc65)c4)cc3)c3ccccc3-c3cccc4c3oc3c5ccccc5ccc43)cc2)cc1 ; c1cc(-c2ccc(N(c3ccc(-c4cccc5ccccc45)cc3)c3ccccc3-c3cccc4c3oc3c5ccccc5ccc43)cc2)cc(-n2c3ccccc3c3ccccc32)c1 ; c1cc(-c2ccc(N(c3ccc(-c4ccc5ccccc5c4)cc3)c3ccccc3-c3cccc4c3oc3c5ccccc5ccc43)cc2)cc(-n2c3ccccc3c3ccccc32)c1]

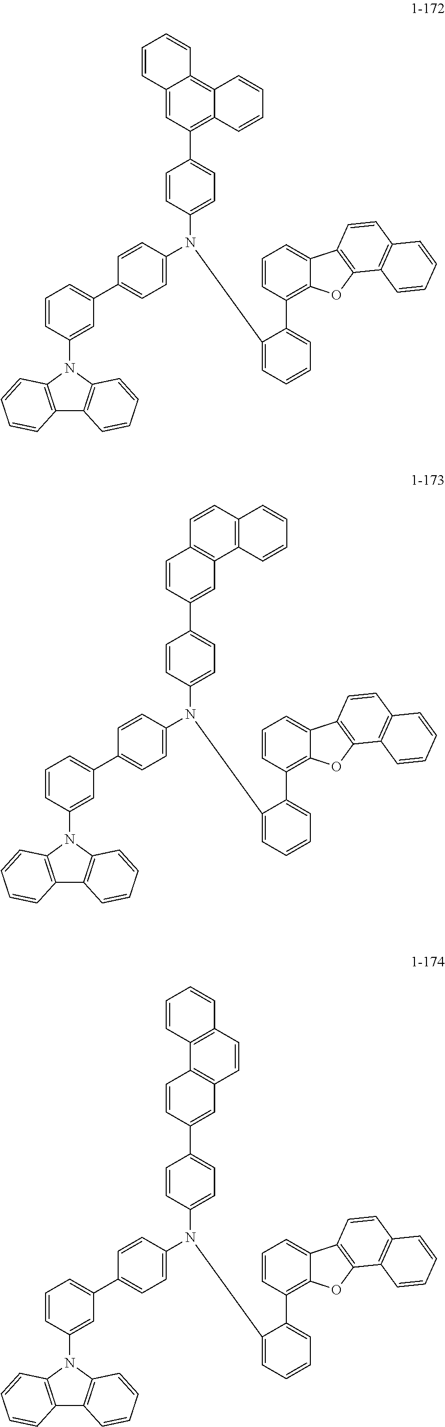 [SMILES: c1cc(-c2ccc(N(c3ccc(-c4cc5ccccc5c5ccccc45)cc3)c3ccccc3-c3cccc4c3oc3c5ccccc5ccc43)cc2)cc(-n2c3ccccc3c3ccccc32)c1 ; c1cc(-c2ccc(N(c3ccc(-c4ccc5ccc6ccccc6c5c4)cc3)c3ccccc3-c3cccc4c3oc3c5ccccc5ccc43)cc2)cc(-n2c3ccccc3c3ccccc32)c1 ; c1cc(-c2ccc(N(c3ccc(-c4ccc5c(ccc6ccccc65)c4)cc3)c3ccccc3-c3cccc4c3oc3c5ccccc5ccc43)cc2)cc(-n2c3ccccc3c3ccccc32)c1]

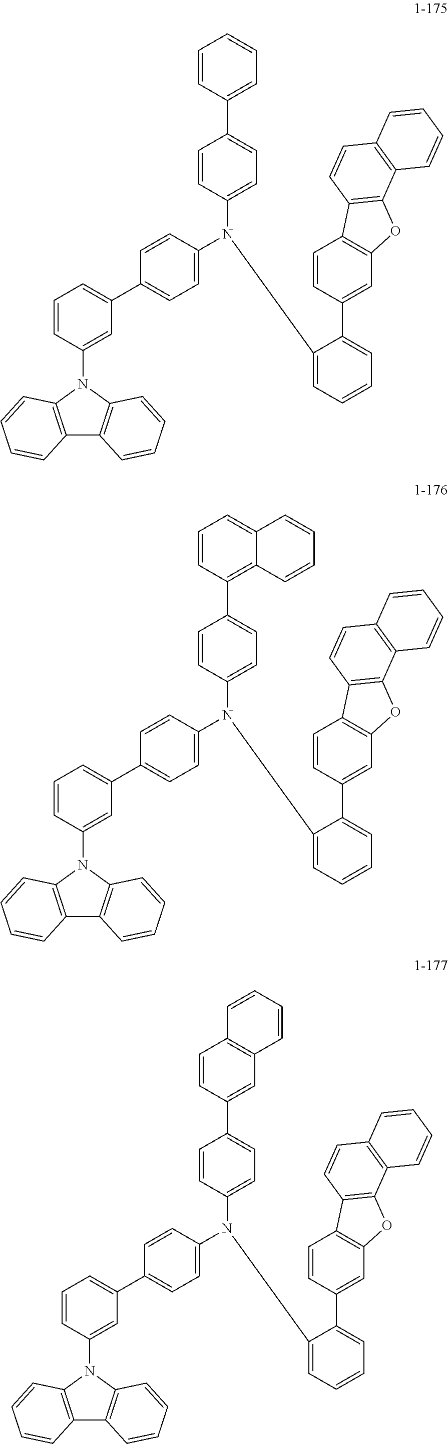 [SMILES: c1ccc(-c2ccc(N(c3ccc(-c4cccc(-n5c6ccccc6c6ccccc65)c4)cc3)c3ccccc3-c3ccc4c(c3)oc3c5ccccc5ccc43)cc2)cc1 ; c1cc(-c2ccc(N(c3ccc(-c4cccc5ccccc45)cc3)c3ccccc3-c3ccc4c(c3)oc3c5ccccc5ccc43)cc2)cc(-n2c3ccccc3c3ccccc32)c1 ; c1cc(-c2ccc(N(c3ccc(-c4ccc5ccccc5c4)cc3)c3ccccc3-c3ccc4c(c3)oc3c5ccccc5ccc43)cc2)cc(-n2c3ccccc3c3ccccc32)c1]

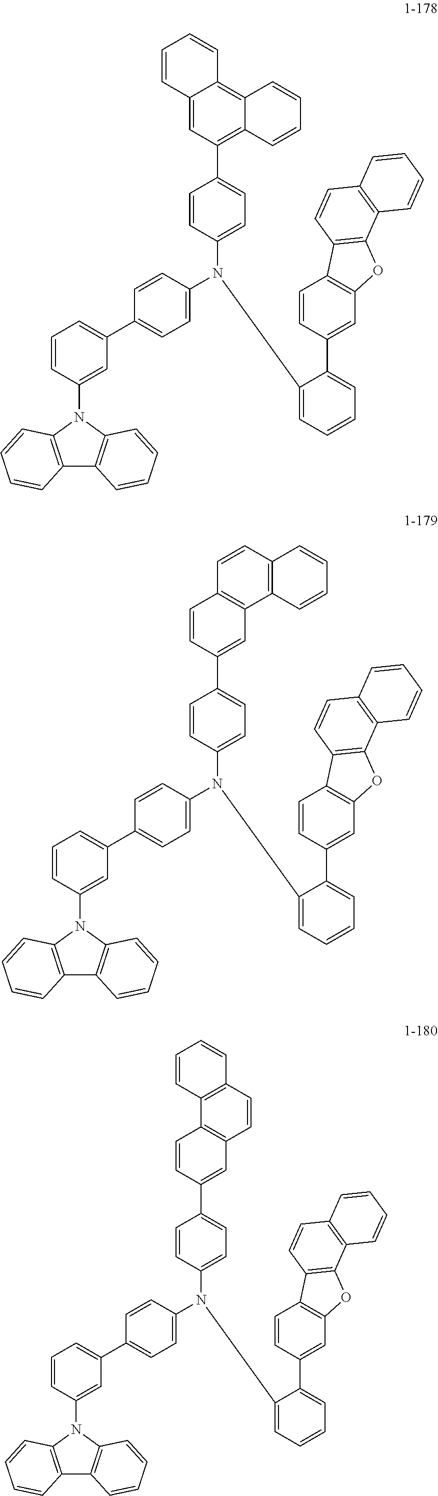 [SMILES: c1cc(-c2ccc(N(c3ccc(-c4cc5ccccc5c5ccccc45)cc3)c3ccccc3-c3ccc4c(c3)oc3c5ccccc5ccc43)cc2)cc(-n2c3ccccc3c3ccccc32)c1 ; c1cc(-c2ccc(N(c3ccc(-c4ccc5ccc6ccccc6c5c4)cc3)c3ccccc3-c3ccc4c(c3)oc3c5ccccc5ccc43)cc2)cc(-n2c3ccccc3c3ccccc32)c1 ; c1cc(-c2ccc(N(c3ccc(-c4ccc5c(ccc6ccccc65)c4)cc3)c3ccccc3-c3ccc4c(c3)oc3c5ccccc5ccc43)cc2)cc(-n2c3ccccc3c3ccccc32)c1]

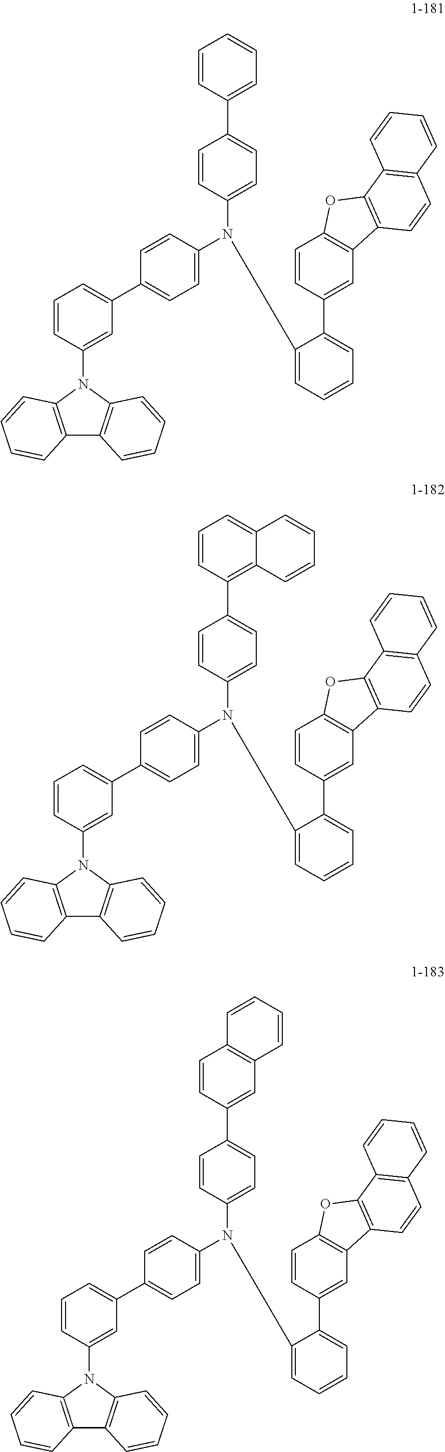 [SMILES: c1ccc(-c2ccc(N(c3ccc(-c4cccc(-n5c6ccccc6c6ccccc65)c4)cc3)c3ccccc3-c3ccc4oc5c6ccccc6ccc5c4c3)cc2)cc1 ; c1cc(-c2ccc(N(c3ccc(-c4cccc5ccccc45)cc3)c3ccccc3-c3ccc4oc5c6ccccc6ccc5c4c3)cc2)cc(-n2c3ccccc3c3ccccc32)c1 ; c1cc(-c2ccc(N(c3ccc(-c4ccc5ccccc5c4)cc3)c3ccccc3-c3ccc4oc5c6ccccc6ccc5c4c3)cc2)cc(-n2c3ccccc3c3ccccc32)c1]

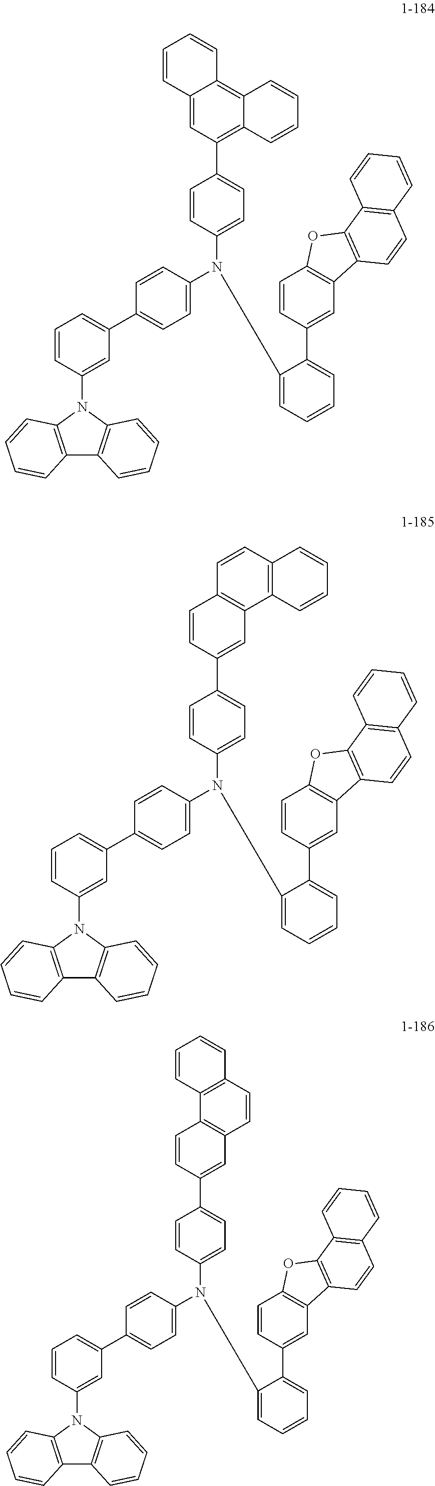 [SMILES: c1cc(-c2ccc(N(c3ccc(-c4cc5ccccc5c5ccccc45)cc3)c3ccccc3-c3ccc4oc5c6ccccc6ccc5c4c3)cc2)cc(-n2c3ccccc3c3ccccc32)c1 ; c1cc(-c2ccc(N(c3ccc(-c4ccc5ccc6ccccc6c5c4)cc3)c3ccccc3-c3ccc4oc5c6ccccc6ccc5c4c3)cc2)cc(-n2c3ccccc3c3ccccc32)c1 ; c1cc(-c2ccc(N(c3ccc(-c4ccc5c(ccc6ccccc65)c4)cc3)c3ccccc3-c3ccc4oc5c6ccccc6ccc5c4c3)cc2)cc(-n2c3ccccc3c3ccccc32)c1]

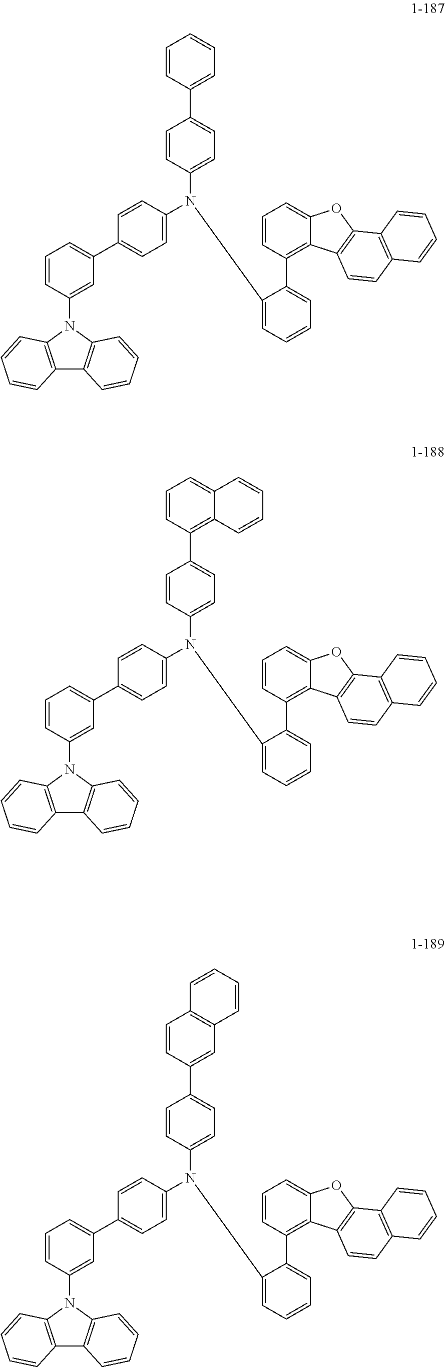 [SMILES: c1ccc(-c2ccc(N(c3ccc(-c4cccc(-n5c6ccccc6c6ccccc65)c4)cc3)c3ccccc3-c3cccc4oc5c6ccccc6ccc5c34)cc2)cc1 ; c1cc(-c2ccc(N(c3ccc(-c4cccc5ccccc45)cc3)c3ccccc3-c3cccc4oc5c6ccccc6ccc5c34)cc2)cc(-n2c3ccccc3c3ccccc32)c1 ; c1cc(-c2ccc(N(c3ccc(-c4ccc5ccccc5c4)cc3)c3ccccc3-c3cccc4oc5c6ccccc6ccc5c34)cc2)cc(-n2c3ccccc3c3ccccc32)c1]

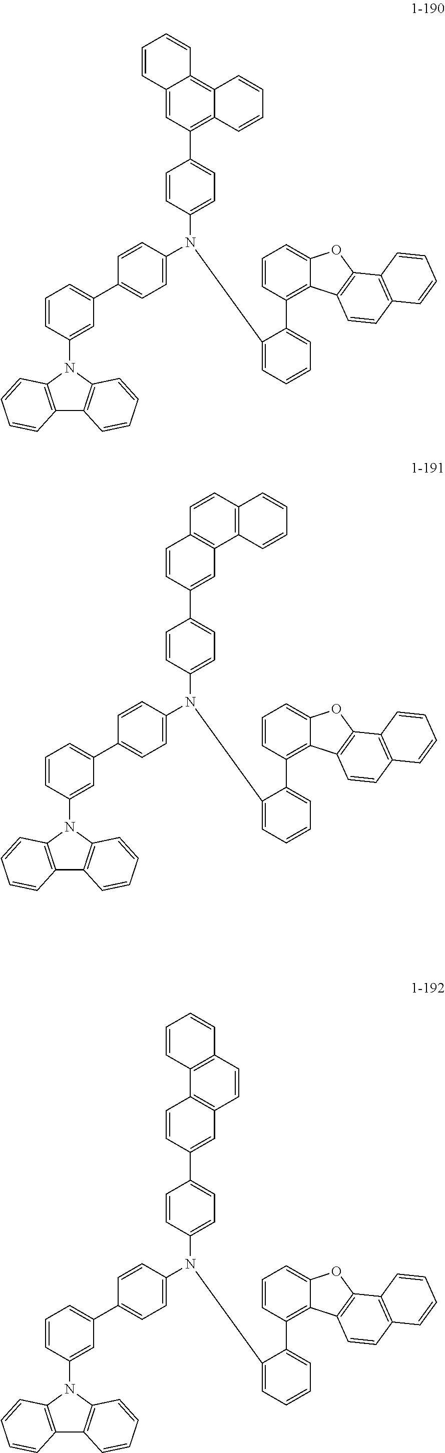 [SMILES: c1cc(-c2ccc(N(c3ccc(-c4cc5ccccc5c5ccccc45)cc3)c3ccccc3-c3cccc4oc5c6ccccc6ccc5c34)cc2)cc(-n2c3ccccc3c3ccccc32)c1 ; c1cc(-c2ccc(N(c3ccc(-c4ccc5ccc6ccccc6c5c4)cc3)c3ccccc3-c3cccc4oc5c6ccccc6ccc5c34)cc2)cc(-n2c3ccccc3c3ccccc32)c1 ; c1cc(-c2ccc(N(c3ccc(-c4ccc5c(ccc6ccccc65)c4)cc3)c3ccccc3-c3cccc4oc5c6ccccc6ccc5c34)cc2)cc(-n2c3ccccc3c3ccccc32)c1]

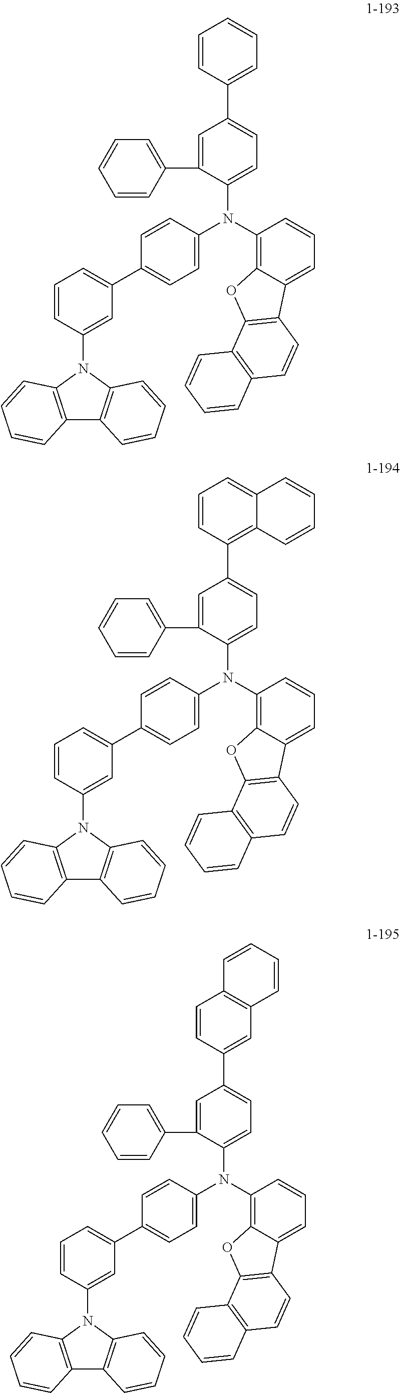 [SMILES: c1ccc(-c2ccc(N(c3ccc(-c4cccc(-n5c6ccccc6c6ccccc65)c4)cc3)c3cccc4c3oc3c5ccccc5ccc43)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2cccc3c2oc2c4ccccc4ccc32)cc1 ; c1ccc(-c2cc(-c3ccc4ccccc4c3)ccc2N(c2ccc(-c3cccc(-n4c5ccccc5c5ccccc54)c3)cc2)c2cccc3c2oc2c4ccccc4ccc32)cc1]

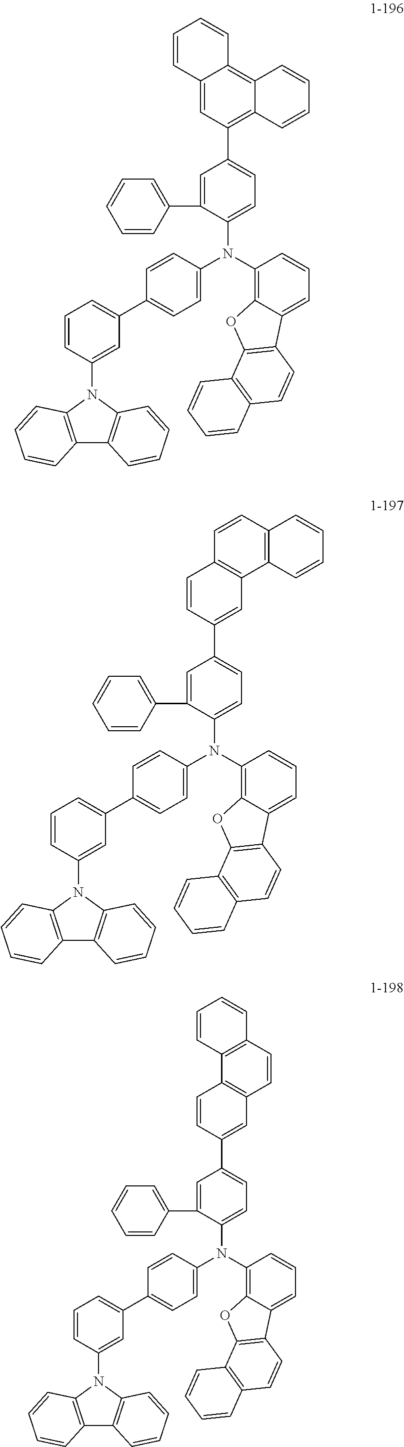 [SMILES: c1ccc(-c2cc(-c3cc4ccccc4c4ccccc34)ccc2N(c2ccc(-c3cccc(-n4c5ccccc5c5ccccc54)c3)cc2)c2cccc3c2oc2c4ccccc4ccc32)cc1 ; c1ccc(-c2cc(-c3ccc4ccc5ccccc5c4c3)ccc2N(c2ccc(-c3cccc(-n4c5ccccc5c5ccccc54)c3)cc2)c2cccc3c2oc2c4ccccc4ccc32)cc1 ; c1ccc(-c2cc(-c3ccc4c(ccc5ccccc54)c3)ccc2N(c2ccc(-c3cccc(-n4c5ccccc5c5ccccc54)c3)cc2)c2cccc3c2oc2c4ccccc4ccc32)cc1]

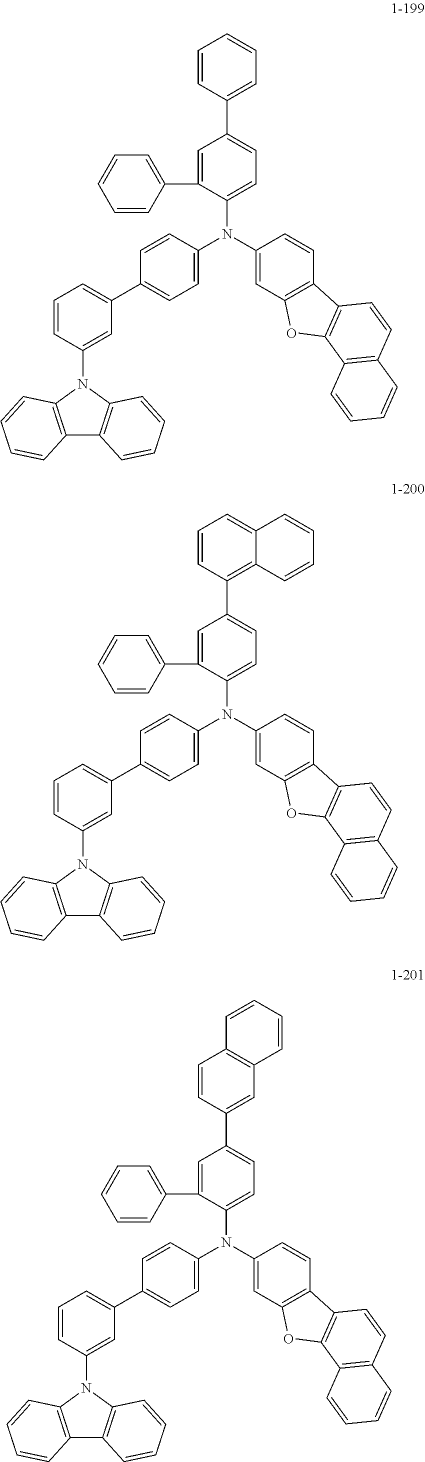 [SMILES: c1ccc(-c2ccc(N(c3ccc(-c4cccc(-n5c6ccccc6c6ccccc65)c4)cc3)c3ccc4c(c3)oc3c5ccccc5ccc43)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2ccc3c(c2)oc2c4ccccc4ccc32)cc1 ; c1ccc(-c2cc(-c3ccc4ccccc4c3)ccc2N(c2ccc(-c3cccc(-n4c5ccccc5c5ccccc54)c3)cc2)c2ccc3c(c2)oc2c4ccccc4ccc32)cc1]

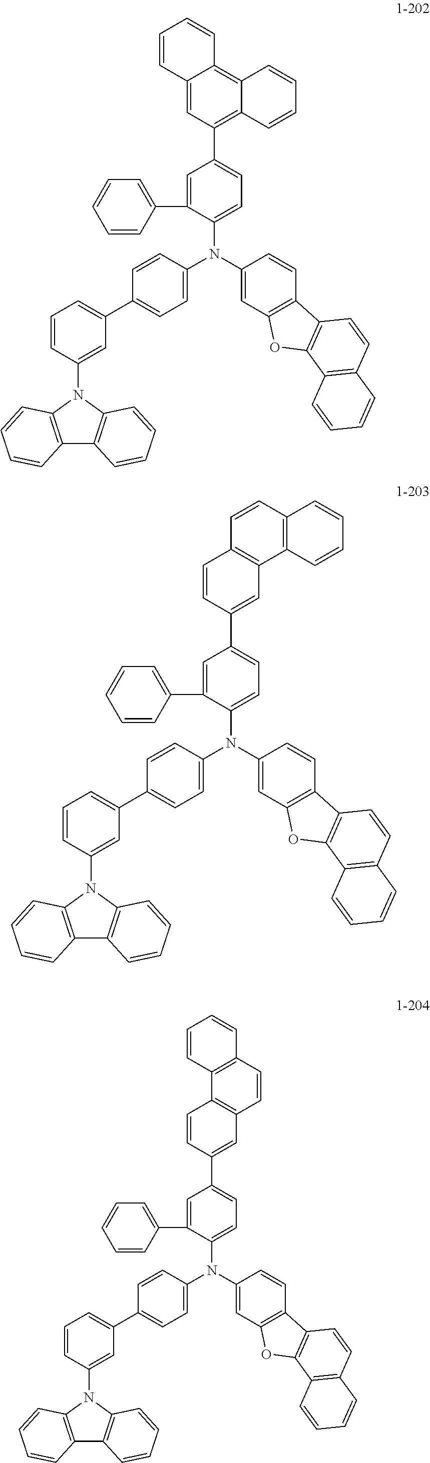 [SMILES: c1ccc(-c2cc(-c3cc4ccccc4c4ccccc34)ccc2N(c2ccc(-c3cccc(-n4c5ccccc5c5ccccc54)c3)cc2)c2ccc3c(c2)oc2c4ccccc4ccc32)cc1 ; c1ccc(-c2cc(-c3ccc4ccc5ccccc5c4c3)ccc2N(c2ccc(-c3cccc(-n4c5ccccc5c5ccccc54)c3)cc2)c2ccc3c(c2)oc2c4ccccc4ccc32)cc1 ; c1ccc(-c2cc(-c3ccc4c(ccc5ccccc54)c3)ccc2N(c2ccc(-c3cccc(-n4c5ccccc5c5ccccc54)c3)cc2)c2ccc3c(c2)oc2c4ccccc4ccc32)cc1]

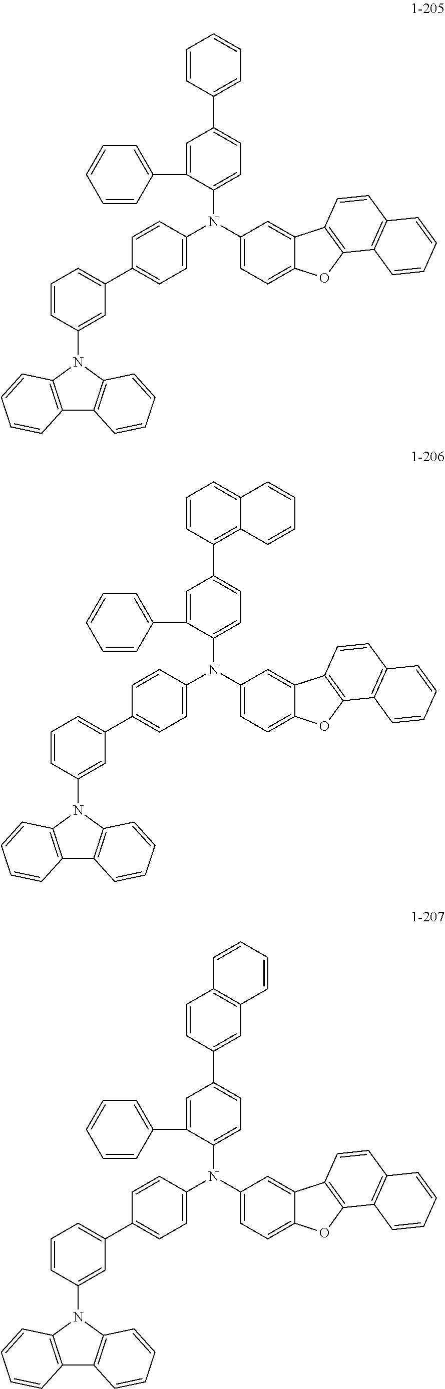 [SMILES: c1ccc(-c2ccc(N(c3ccc(-c4cccc(-n5c6ccccc6c6ccccc65)c4)cc3)c3ccc4oc5c6ccccc6ccc5c4c3)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2ccc3oc4c5ccccc5ccc4c3c2)cc1 ; c1ccc(-c2cc(-c3ccc4ccccc4c3)ccc2N(c2ccc(-c3cccc(-n4c5ccccc5c5ccccc54)c3)cc2)c2ccc3oc4c5ccccc5ccc4c3c2)cc1]

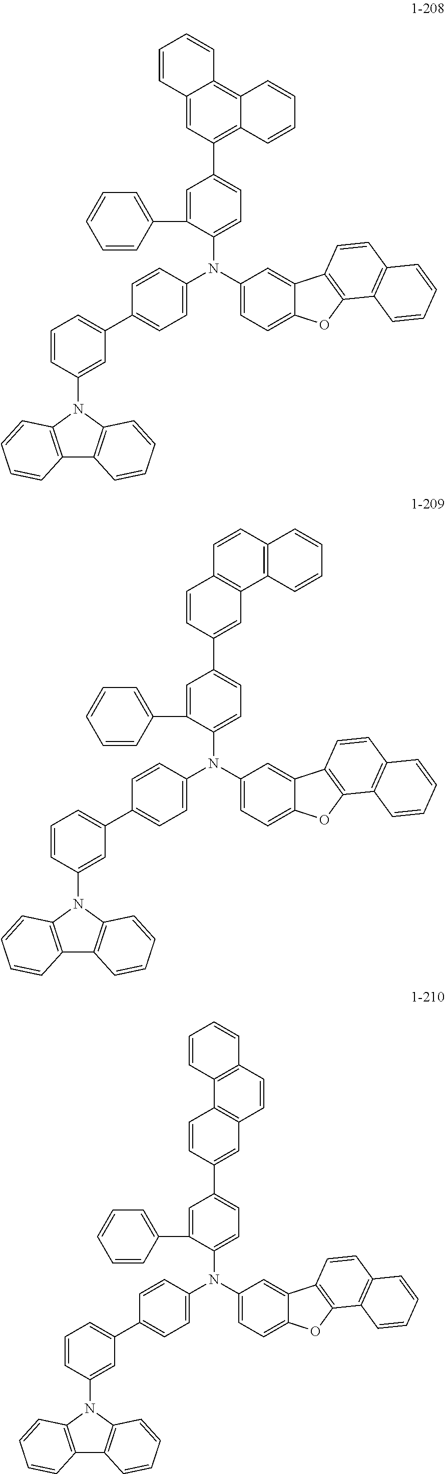 [SMILES: c1ccc(-c2cc(-c3cc4ccccc4c4ccccc34)ccc2N(c2ccc(-c3cccc(-n4c5ccccc5c5ccccc54)c3)cc2)c2ccc3oc4c5ccccc5ccc4c3c2)cc1 ; c1ccc(-c2cc(-c3ccc4ccc5ccccc5c4c3)ccc2N(c2ccc(-c3cccc(-n4c5ccccc5c5ccccc54)c3)cc2)c2ccc3oc4c5ccccc5ccc4c3c2)cc1 ; c1ccc(-c2cc(-c3ccc4c(ccc5ccccc54)c3)ccc2N(c2ccc(-c3cccc(-n4c5ccccc5c5ccccc54)c3)cc2)c2ccc3oc4c5ccccc5ccc4c3c2)cc1]

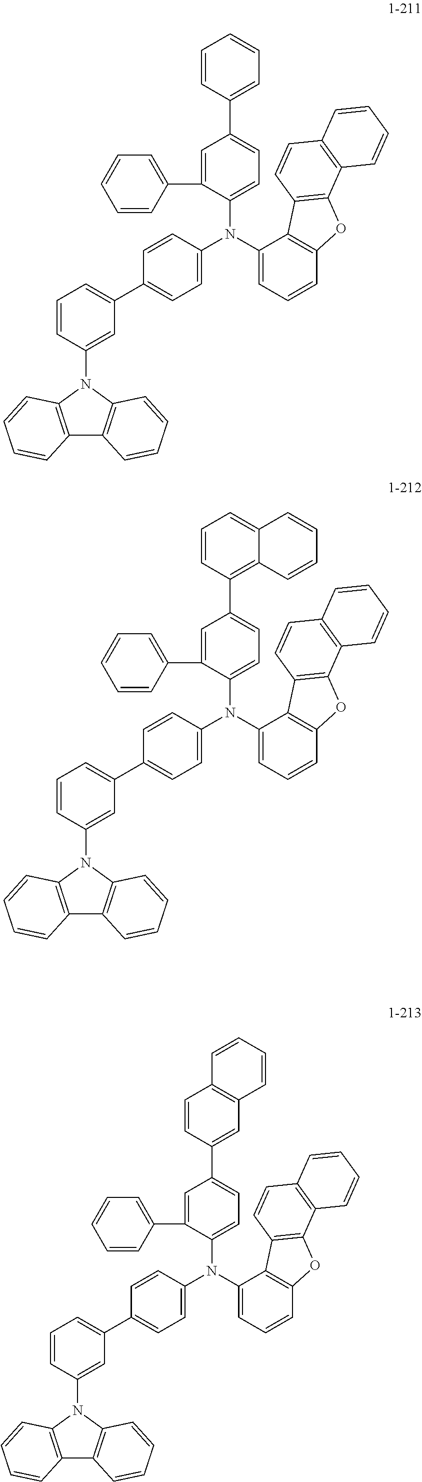 [SMILES: c1ccc(-c2ccc(N(c3ccc(-c4cccc(-n5c6ccccc6c6ccccc65)c4)cc3)c3cccc4oc5c6ccccc6ccc5c34)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2cccc3oc4c5ccccc5ccc4c23)cc1 ; c1ccc(-c2cc(-c3ccc4ccccc4c3)ccc2N(c2ccc(-c3cccc(-n4c5ccccc5c5ccccc54)c3)cc2)c2cccc3oc4c5ccccc5ccc4c23)cc1]

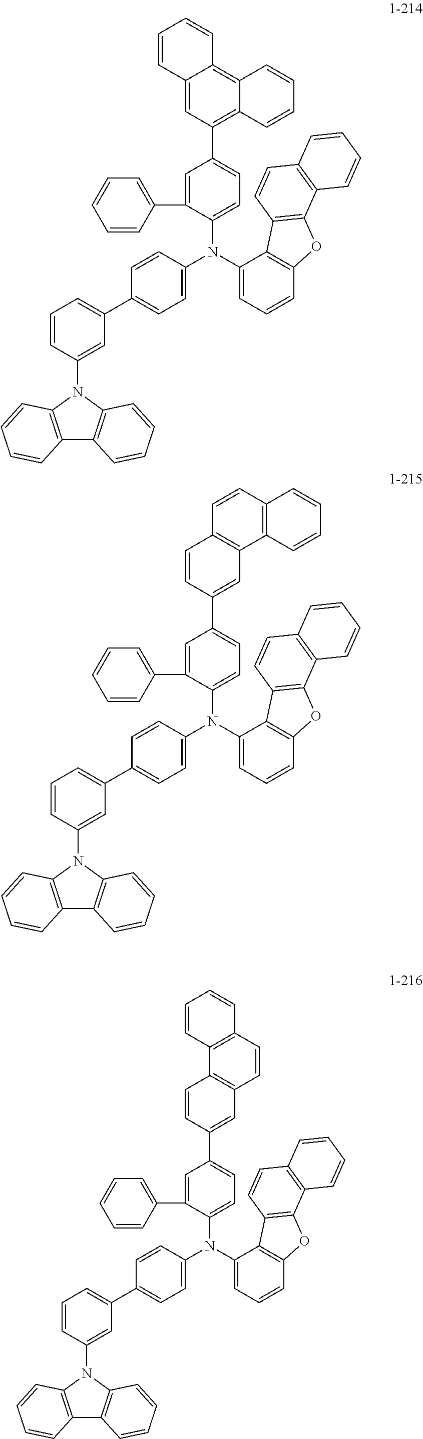 [SMILES: c1ccc(-c2cc(-c3cc4ccccc4c4ccccc34)ccc2N(c2ccc(-c3cccc(-n4c5ccccc5c5ccccc54)c3)cc2)c2cccc3oc4c5ccccc5ccc4c23)cc1 ; c1ccc(-c2cc(-c3ccc4ccc5ccccc5c4c3)ccc2N(c2ccc(-c3cccc(-n4c5ccccc5c5ccccc54)c3)cc2)c2cccc3oc4c5ccccc5ccc4c23)cc1 ; c1ccc(-c2cc(-c3ccc4c(ccc5ccccc54)c3)ccc2N(c2ccc(-c3cccc(-n4c5ccccc5c5ccccc54)c3)cc2)c2cccc3oc4c5ccccc5ccc4c23)cc1]

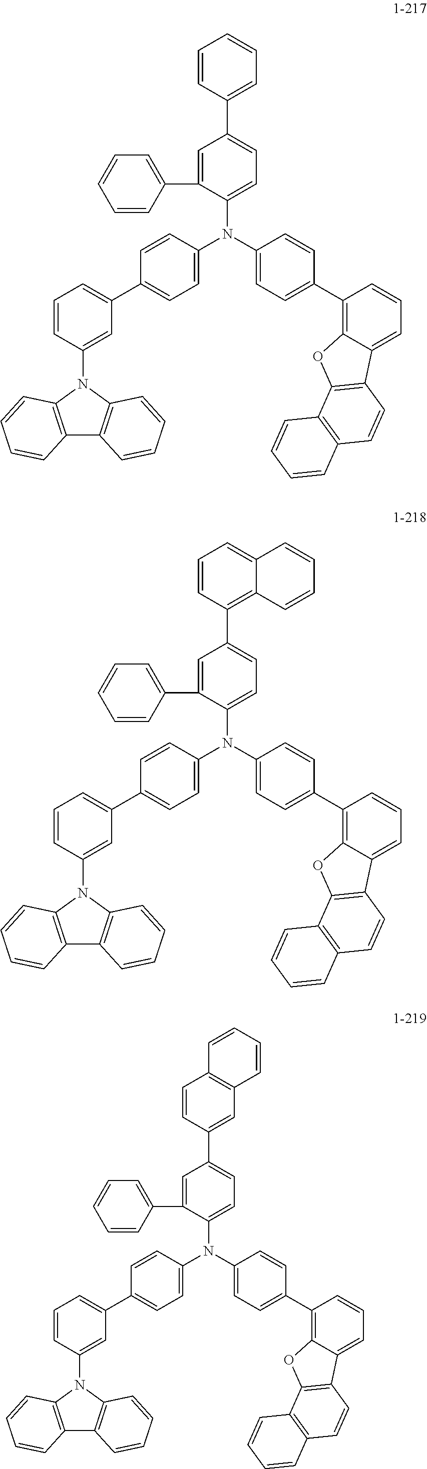 [SMILES: c1ccc(-c2ccc(N(c3ccc(-c4cccc(-n5c6ccccc6c6ccccc65)c4)cc3)c3ccc(-c4cccc5c4oc4c6ccccc6ccc54)cc3)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2ccc(-c3cccc4c3oc3c5ccccc5ccc43)cc2)cc1 ; c1ccc(-c2cc(-c3ccc4ccccc4c3)ccc2N(c2ccc(-c3cccc(-n4c5ccccc5c5ccccc54)c3)cc2)c2ccc(-c3cccc4c3oc3c5ccccc5ccc43)cc2)cc1]

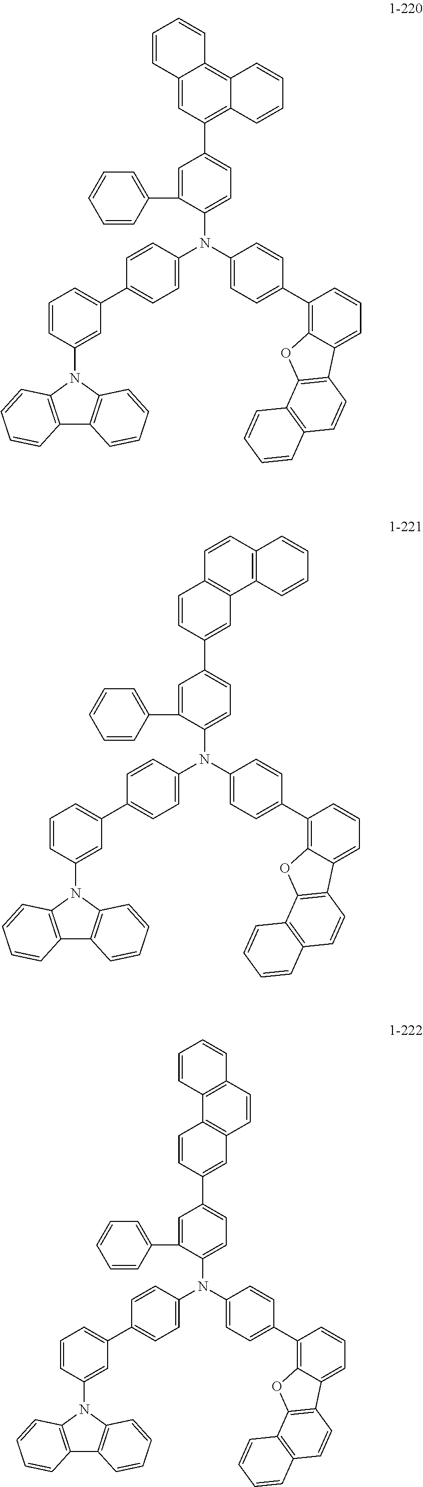 [SMILES: c1ccc(-c2cc(-c3cc4ccccc4c4ccccc34)ccc2N(c2ccc(-c3cccc(-n4c5ccccc5c5ccccc54)c3)cc2)c2ccc(-c3cccc4c3oc3c5ccccc5ccc43)cc2)cc1 ; c1ccc(-c2cc(-c3ccc4ccc5ccccc5c4c3)ccc2N(c2ccc(-c3cccc(-n4c5ccccc5c5ccccc54)c3)cc2)c2ccc(-c3cccc4c3oc3c5ccccc5ccc43)cc2)cc1 ; c1ccc(-c2cc(-c3ccc4c(ccc5ccccc54)c3)ccc2N(c2ccc(-c3cccc(-n4c5ccccc5c5ccccc54)c3)cc2)c2ccc(-c3cccc4c3oc3c5ccccc5ccc43)cc2)cc1]

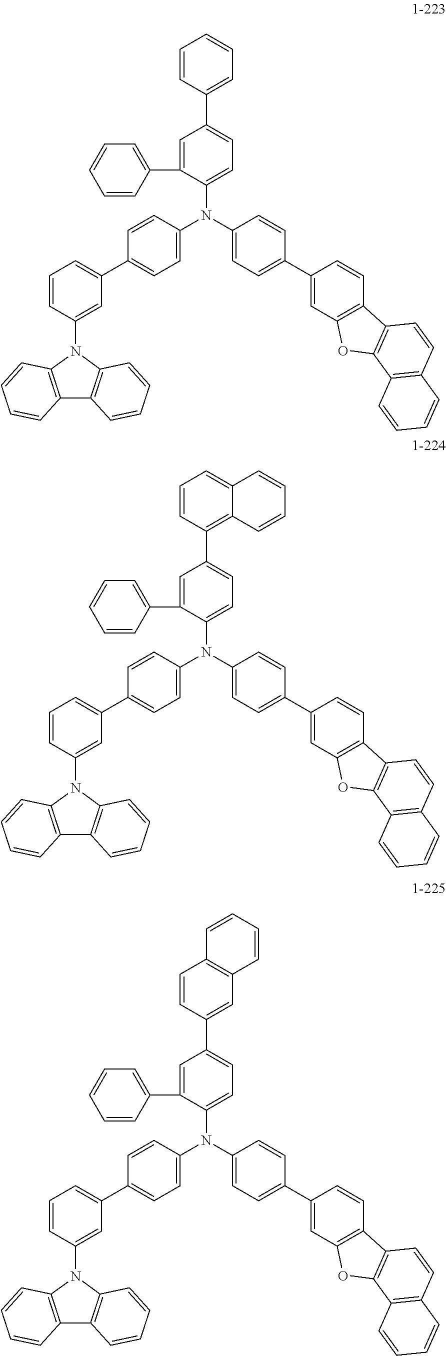 [SMILES: c1ccc(-c2ccc(N(c3ccc(-c4cccc(-n5c6ccccc6c6ccccc65)c4)cc3)c3ccc(-c4ccc5c(c4)oc4c6ccccc6ccc54)cc3)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2ccc(-c3ccc4c(c3)oc3c5ccccc5ccc43)cc2)cc1 ; c1ccc(-c2cc(-c3ccc4ccccc4c3)ccc2N(c2ccc(-c3cccc(-n4c5ccccc5c5ccccc54)c3)cc2)c2ccc(-c3ccc4c(c3)oc3c5ccccc5ccc43)cc2)cc1]

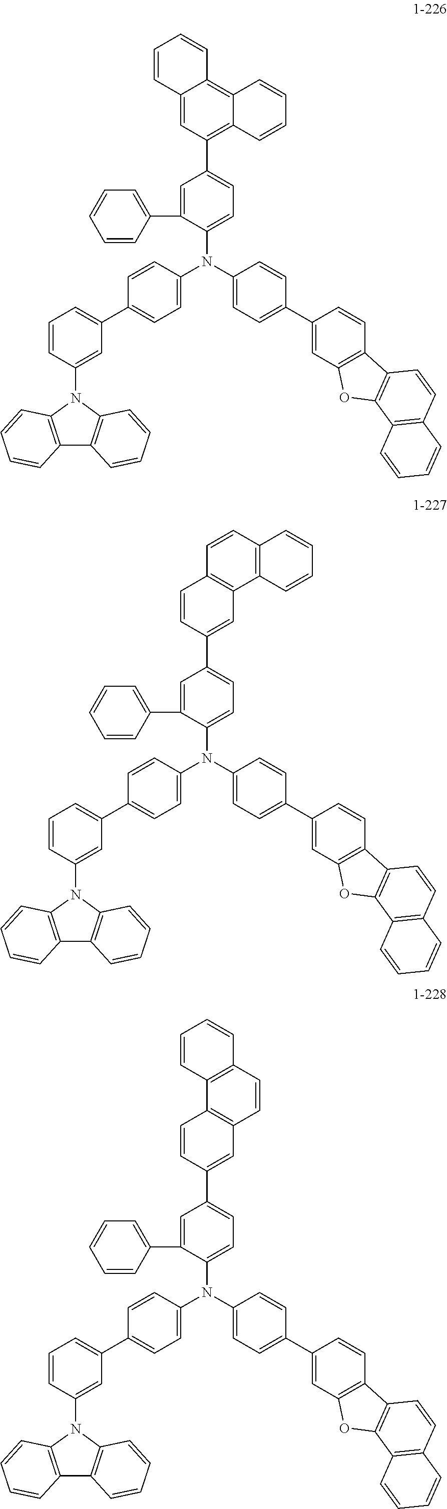 [SMILES: c1ccc(-c2cc(-c3cc4ccccc4c4ccccc34)ccc2N(c2ccc(-c3cccc(-n4c5ccccc5c5ccccc54)c3)cc2)c2ccc(-c3ccc4c(c3)oc3c5ccccc5ccc43)cc2)cc1 ; c1ccc(-c2cc(-c3ccc4ccc5ccccc5c4c3)ccc2N(c2ccc(-c3cccc(-n4c5ccccc5c5ccccc54)c3)cc2)c2ccc(-c3ccc4c(c3)oc3c5ccccc5ccc43)cc2)cc1 ; c1ccc(-c2cc(-c3ccc4c(ccc5ccccc54)c3)ccc2N(c2ccc(-c3cccc(-n4c5ccccc5c5ccccc54)c3)cc2)c2ccc(-c3ccc4c(c3)oc3c5ccccc5ccc43)cc2)cc1]

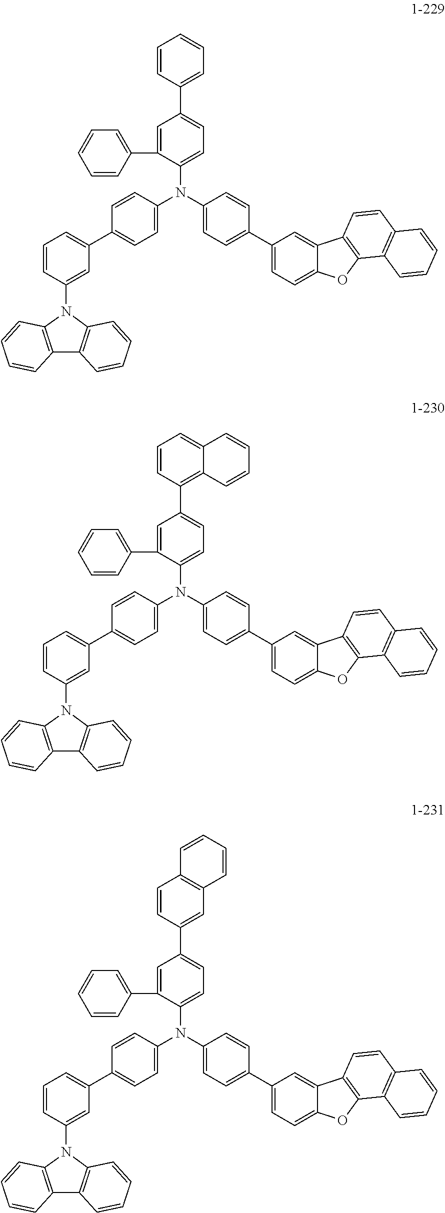 [SMILES: c1ccc(-c2ccc(N(c3ccc(-c4cccc(-n5c6ccccc6c6ccccc65)c4)cc3)c3ccc(-c4ccc5oc6c7ccccc7ccc6c5c4)cc3)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2ccc(-c3ccc4oc5c6ccccc6ccc5c4c3)cc2)cc1 ; c1ccc(-c2cc(-c3ccc4ccccc4c3)ccc2N(c2ccc(-c3cccc(-n4c5ccccc5c5ccccc54)c3)cc2)c2ccc(-c3ccc4oc5c6ccccc6ccc5c4c3)cc2)cc1]

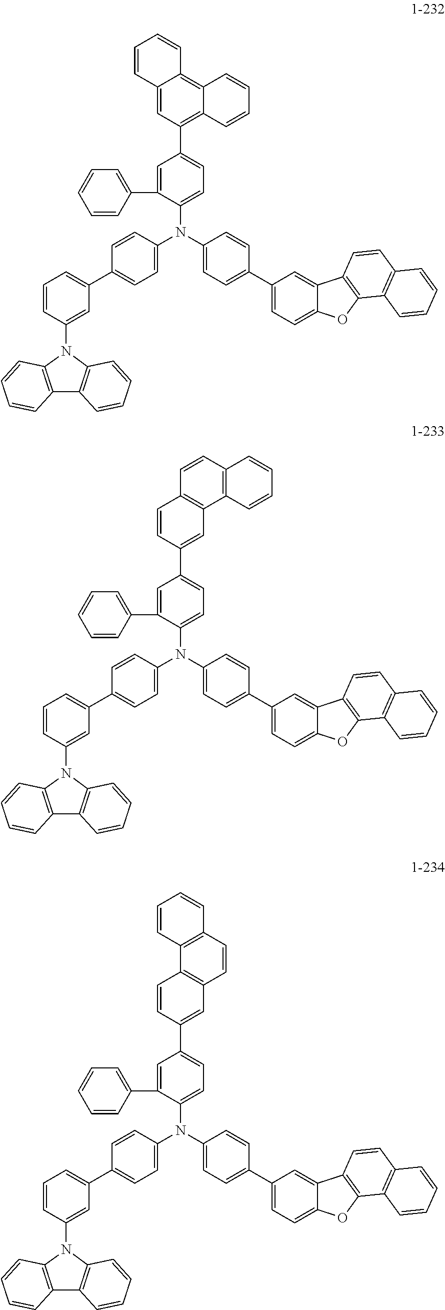 [SMILES: c1ccc(-c2cc(-c3cc4ccccc4c4ccccc34)ccc2N(c2ccc(-c3cccc(-n4c5ccccc5c5ccccc54)c3)cc2)c2ccc(-c3ccc4oc5c6ccccc6ccc5c4c3)cc2)cc1 ; c1ccc(-c2cc(-c3ccc4ccc5ccccc5c4c3)ccc2N(c2ccc(-c3cccc(-n4c5ccccc5c5ccccc54)c3)cc2)c2ccc(-c3ccc4oc5c6ccccc6ccc5c4c3)cc2)cc1 ; c1ccc(-c2cc(-c3ccc4c(ccc5ccccc54)c3)ccc2N(c2ccc(-c3cccc(-n4c5ccccc5c5ccccc54)c3)cc2)c2ccc(-c3ccc4oc5c6ccccc6ccc5c4c3)cc2)cc1]

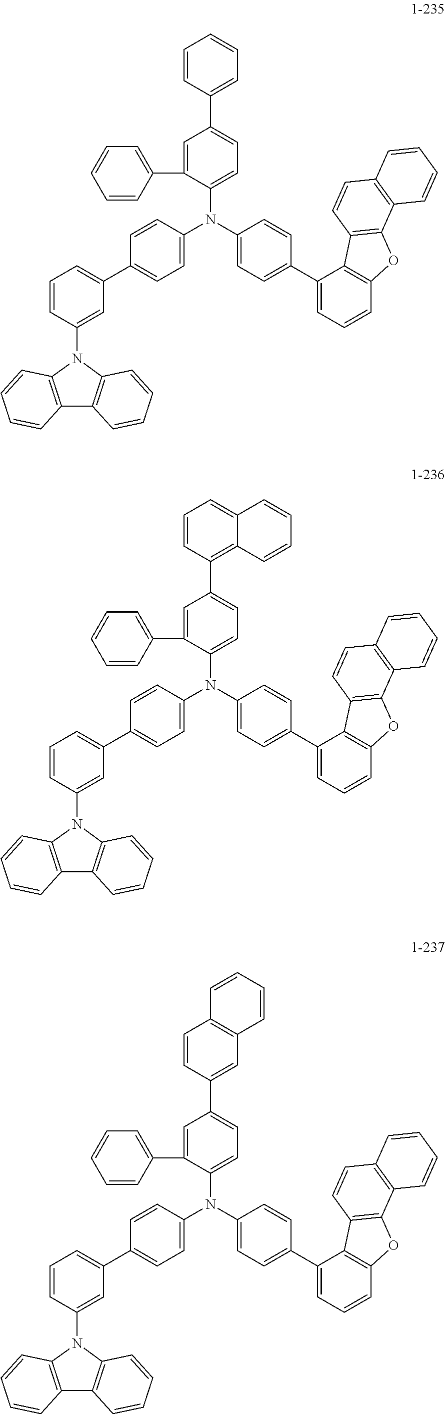 [SMILES: c1ccc(-c2ccc(N(c3ccc(-c4cccc(-n5c6ccccc6c6ccccc65)c4)cc3)c3ccc(-c4cccc5oc6c7ccccc7ccc6c45)cc3)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2ccc(-c3cccc4oc5c6ccccc6ccc5c34)cc2)cc1 ; c1ccc(-c2cc(-c3ccc4ccccc4c3)ccc2N(c2ccc(-c3cccc(-n4c5ccccc5c5ccccc54)c3)cc2)c2ccc(-c3cccc4oc5c6ccccc6ccc5c34)cc2)cc1]

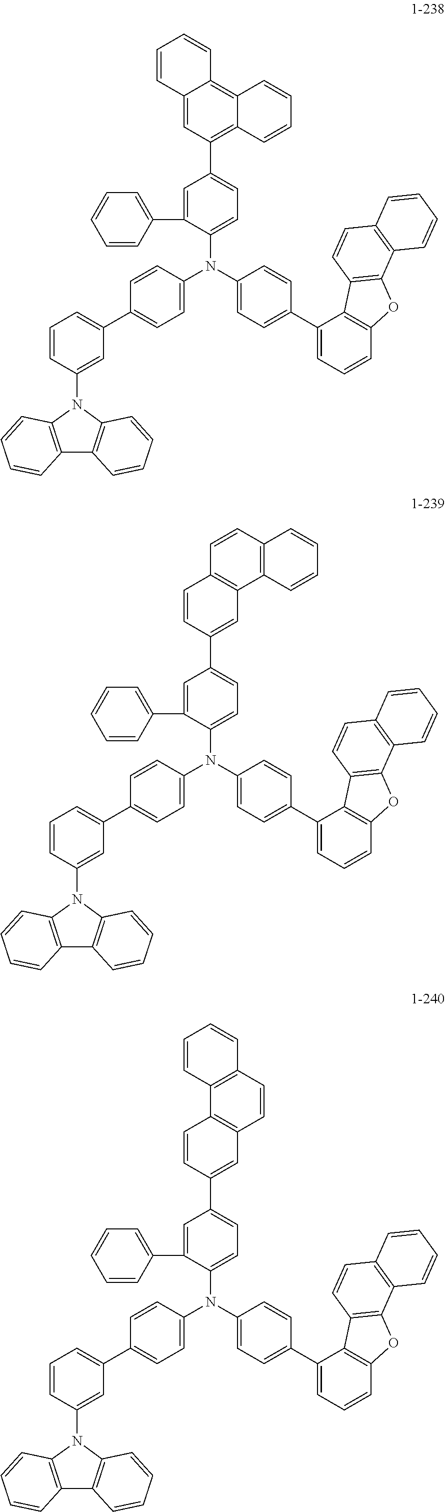 [SMILES: c1ccc(-c2cc(-c3cc4ccccc4c4ccccc34)ccc2N(c2ccc(-c3cccc(-n4c5ccccc5c5ccccc54)c3)cc2)c2ccc(-c3cccc4oc5c6ccccc6ccc5c34)cc2)cc1 ; c1ccc(-c2cc(-c3ccc4ccc5ccccc5c4c3)ccc2N(c2ccc(-c3cccc(-n4c5ccccc5c5ccccc54)c3)cc2)c2ccc(-c3cccc4oc5c6ccccc6ccc5c34)cc2)cc1 ; c1ccc(-c2cc(-c3ccc4c(ccc5ccccc54)c3)ccc2N(c2ccc(-c3cccc(-n4c5ccccc5c5ccccc54)c3)cc2)c2ccc(-c3cccc4oc5c6ccccc6ccc5c34)cc2)cc1]

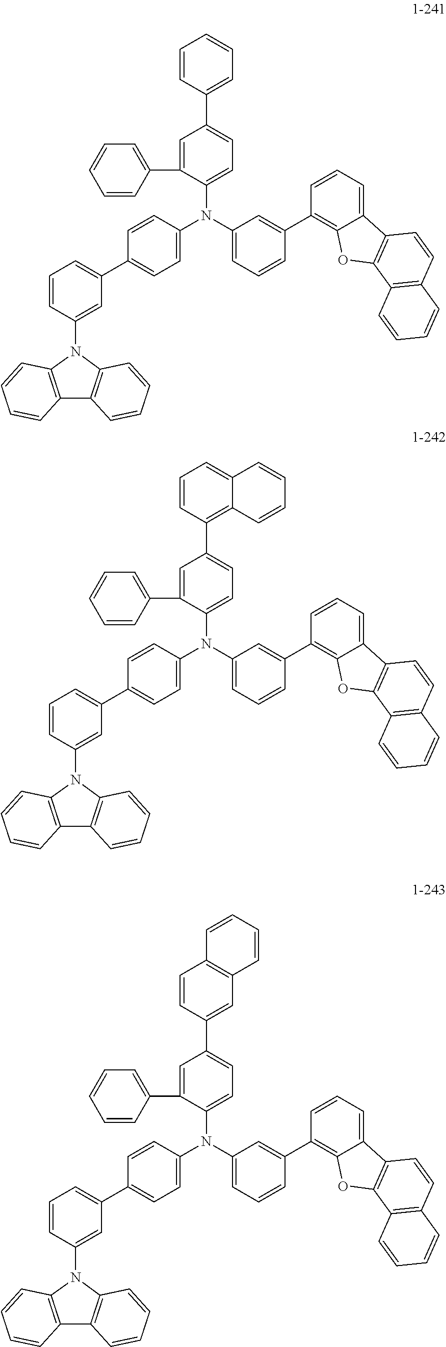 [SMILES: c1ccc(-c2ccc(N(c3ccc(-c4cccc(-n5c6ccccc6c6ccccc65)c4)cc3)c3cccc(-c4cccc5c4oc4c6ccccc6ccc54)c3)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2cccc(-c3cccc4c3oc3c5ccccc5ccc43)c2)cc1 ; c1ccc(-c2cc(-c3ccc4ccccc4c3)ccc2N(c2ccc(-c3cccc(-n4c5ccccc5c5ccccc54)c3)cc2)c2cccc(-c3cccc4c3oc3c5ccccc5ccc43)c2)cc1]

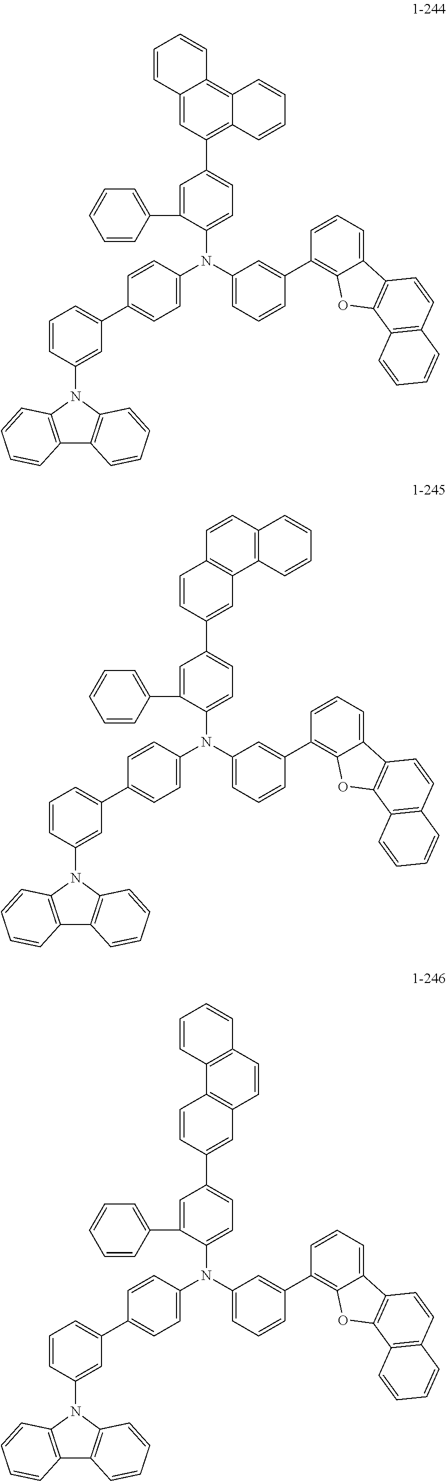 [SMILES: c1ccc(-c2cc(-c3cc4ccccc4c4ccccc34)ccc2N(c2ccc(-c3cccc(-n4c5ccccc5c5ccccc54)c3)cc2)c2cccc(-c3cccc4c3oc3c5ccccc5ccc43)c2)cc1 ; c1ccc(-c2cc(-c3ccc4ccc5ccccc5c4c3)ccc2N(c2ccc(-c3cccc(-n4c5ccccc5c5ccccc54)c3)cc2)c2cccc(-c3cccc4c3oc3c5ccccc5ccc43)c2)cc1 ; c1ccc(-c2cc(-c3ccc4c(ccc5ccccc54)c3)ccc2N(c2ccc(-c3cccc(-n4c5ccccc5c5ccccc54)c3)cc2)c2cccc(-c3cccc4c3oc3c5ccccc5ccc43)c2)cc1]

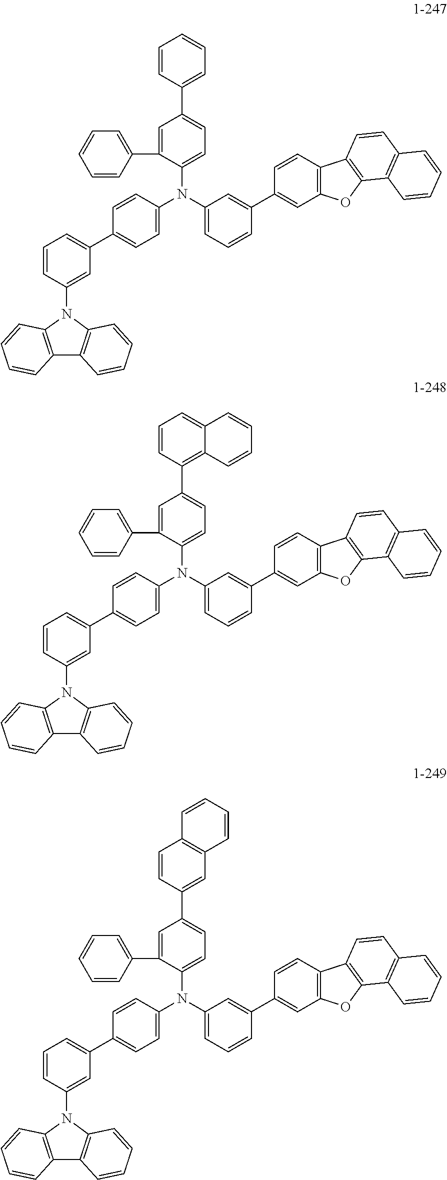 [SMILES: c1ccc(-c2ccc(N(c3ccc(-c4cccc(-n5c6ccccc6c6ccccc65)c4)cc3)c3cccc(-c4ccc5c(c4)oc4c6ccccc6ccc54)c3)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2cccc(-c3ccc4c(c3)oc3c5ccccc5ccc43)c2)cc1 ; c1ccc(-c2cc(-c3ccc4ccccc4c3)ccc2N(c2ccc(-c3cccc(-n4c5ccccc5c5ccccc54)c3)cc2)c2cccc(-c3ccc4c(c3)oc3c5ccccc5ccc43)c2)cc1]

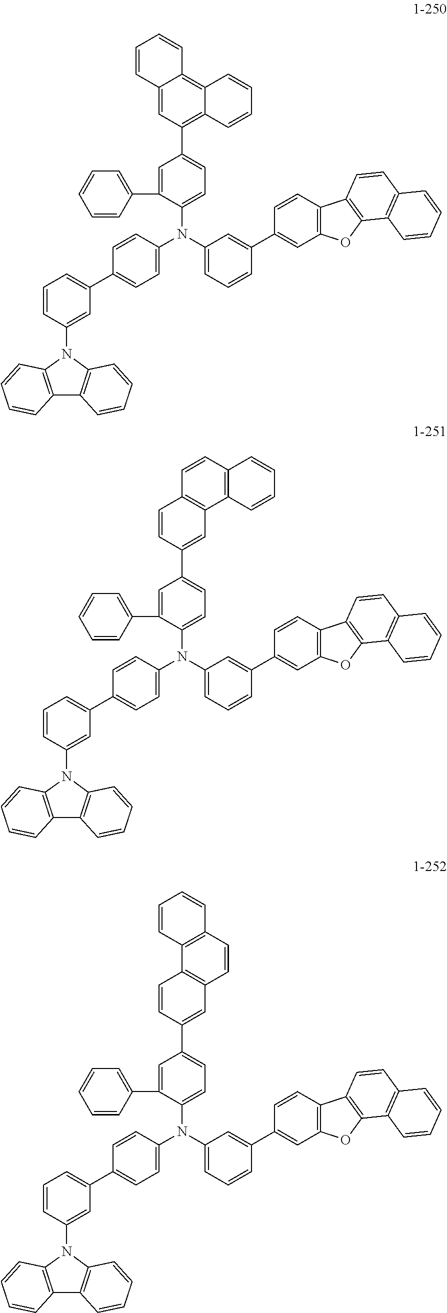 [SMILES: c1ccc(-c2cc(-c3cc4ccccc4c4ccccc34)ccc2N(c2ccc(-c3cccc(-n4c5ccccc5c5ccccc54)c3)cc2)c2cccc(-c3ccc4c(c3)oc3c5ccccc5ccc43)c2)cc1 ; c1ccc(-c2cc(-c3ccc4ccc5ccccc5c4c3)ccc2N(c2ccc(-c3cccc(-n4c5ccccc5c5ccccc54)c3)cc2)c2cccc(-c3ccc4c(c3)oc3c5ccccc5ccc43)c2)cc1 ; c1ccc(-c2cc(-c3ccc4c(ccc5ccccc54)c3)ccc2N(c2ccc(-c3cccc(-n4c5ccccc5c5ccccc54)c3)cc2)c2cccc(-c3ccc4c(c3)oc3c5ccccc5ccc43)c2)cc1]

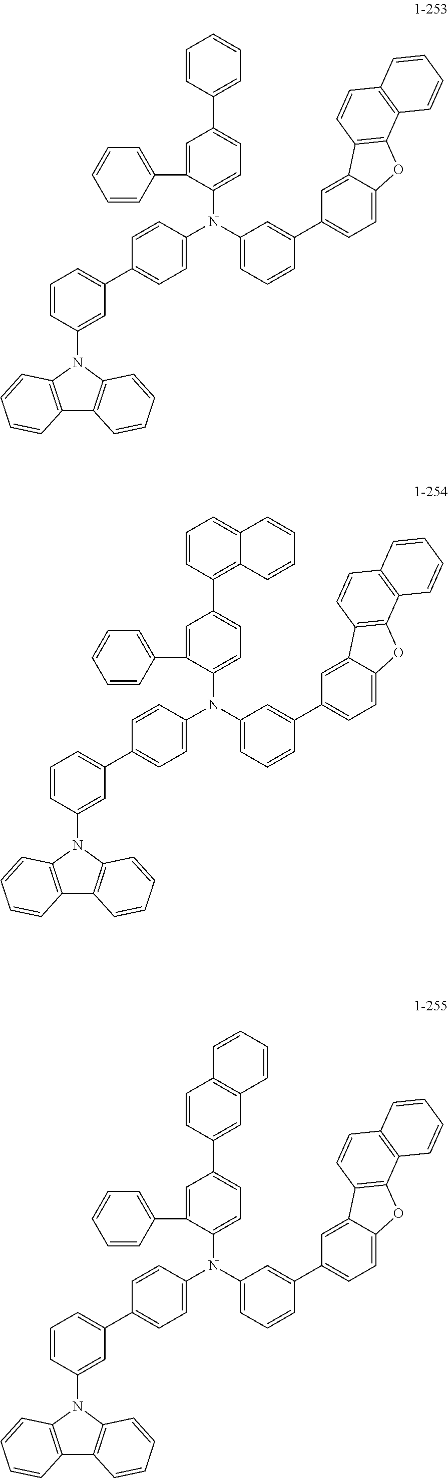 [SMILES: c1ccc(-c2ccc(N(c3ccc(-c4cccc(-n5c6ccccc6c6ccccc65)c4)cc3)c3cccc(-c4ccc5oc6c7ccccc7ccc6c5c4)c3)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2cccc(-c3ccc4oc5c6ccccc6ccc5c4c3)c2)cc1 ; c1ccc(-c2cc(-c3ccc4ccccc4c3)ccc2N(c2ccc(-c3cccc(-n4c5ccccc5c5ccccc54)c3)cc2)c2cccc(-c3ccc4oc5c6ccccc6ccc5c4c3)c2)cc1]

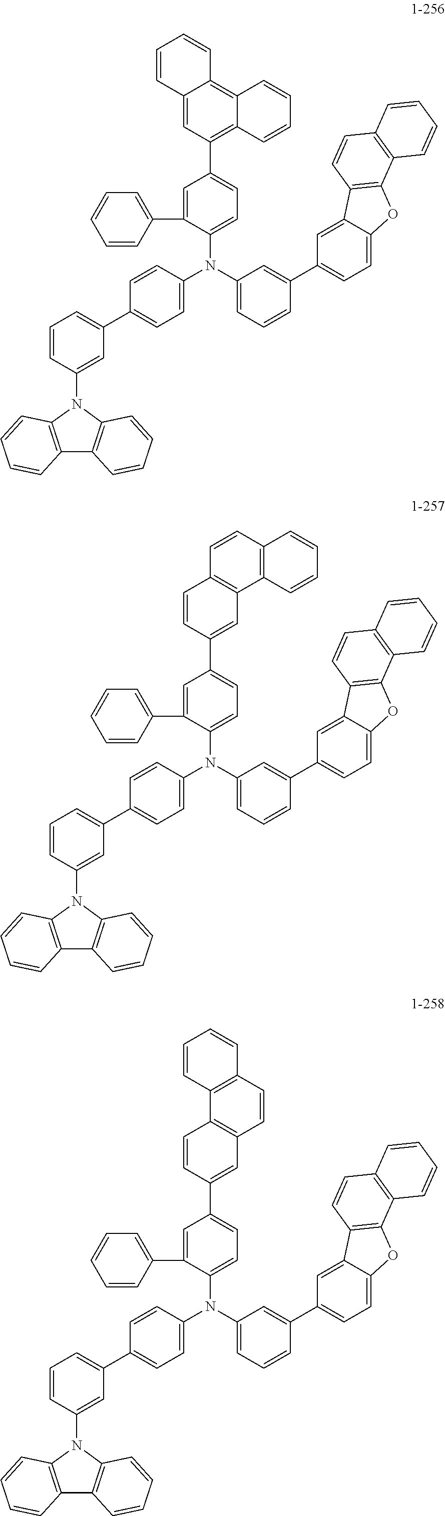 [SMILES: c1ccc(-c2cc(-c3cc4ccccc4c4ccccc34)ccc2N(c2ccc(-c3cccc(-n4c5ccccc5c5ccccc54)c3)cc2)c2cccc(-c3ccc4oc5c6ccccc6ccc5c4c3)c2)cc1 ; c1ccc(-c2cc(-c3ccc4ccc5ccccc5c4c3)ccc2N(c2ccc(-c3cccc(-n4c5ccccc5c5ccccc54)c3)cc2)c2cccc(-c3ccc4oc5c6ccccc6ccc5c4c3)c2)cc1 ; c1ccc(-c2cc(-c3ccc4c(ccc5ccccc54)c3)ccc2N(c2ccc(-c3cccc(-n4c5ccccc5c5ccccc54)c3)cc2)c2cccc(-c3ccc4oc5c6ccccc6ccc5c4c3)c2)cc1]

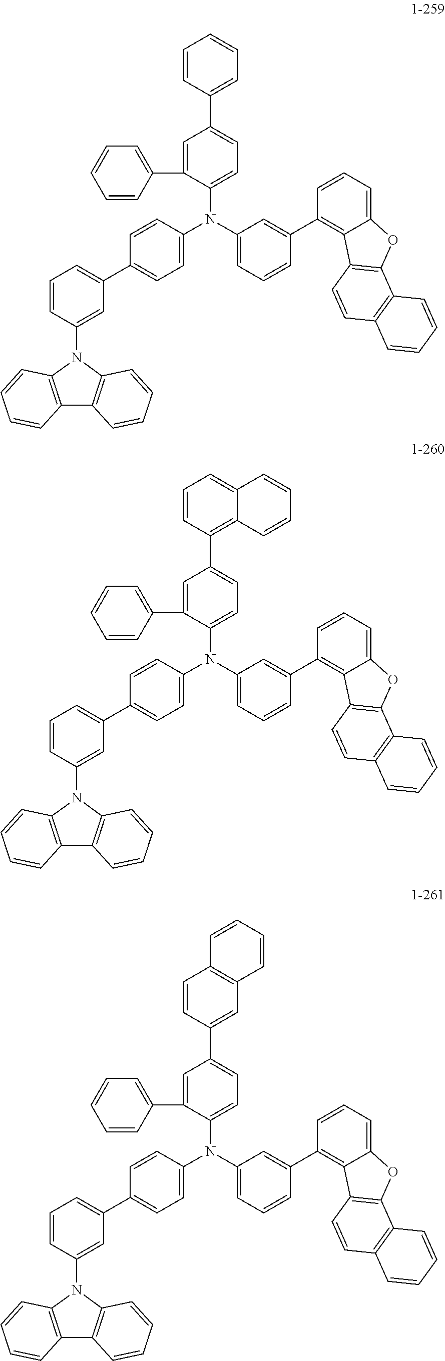 [SMILES: c1ccc(-c2ccc(N(c3ccc(-c4cccc(-n5c6ccccc6c6ccccc65)c4)cc3)c3cccc(-c4cccc5oc6c7ccccc7ccc6c45)c3)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2cccc(-c3cccc4oc5c6ccccc6ccc5c34)c2)cc1 ; c1ccc(-c2cc(-c3ccc4ccccc4c3)ccc2N(c2ccc(-c3cccc(-n4c5ccccc5c5ccccc54)c3)cc2)c2cccc(-c3cccc4oc5c6ccccc6ccc5c34)c2)cc1]

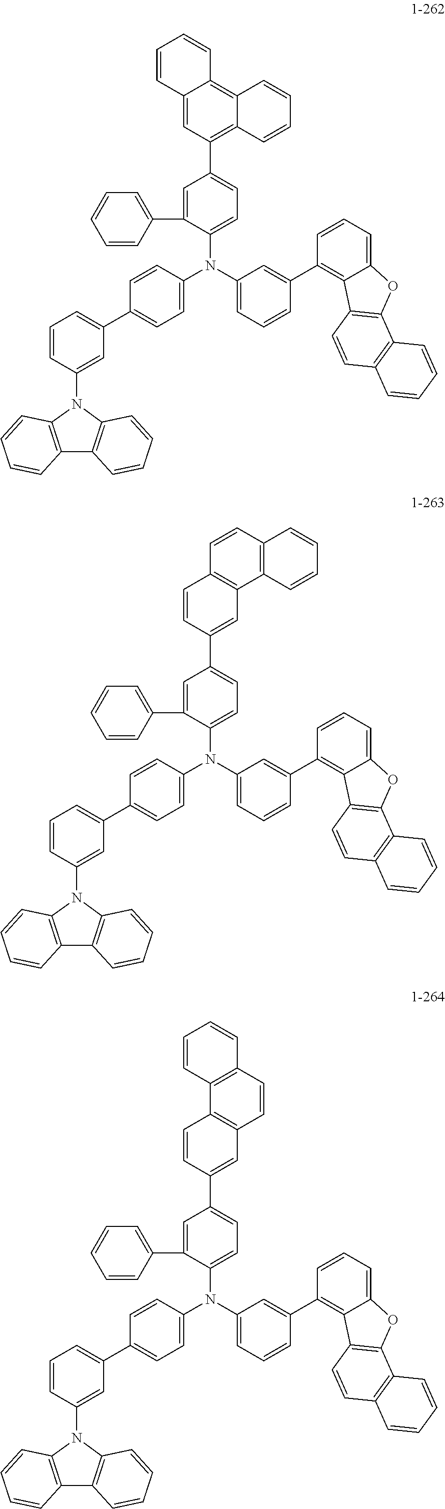 [SMILES: c1ccc(-c2cc(-c3cc4ccccc4c4ccccc34)ccc2N(c2ccc(-c3cccc(-n4c5ccccc5c5ccccc54)c3)cc2)c2cccc(-c3cccc4oc5c6ccccc6ccc5c34)c2)cc1 ; c1ccc(-c2cc(-c3ccc4ccc5ccccc5c4c3)ccc2N(c2ccc(-c3cccc(-n4c5ccccc5c5ccccc54)c3)cc2)c2cccc(-c3cccc4oc5c6ccccc6ccc5c34)c2)cc1 ; c1ccc(-c2cc(-c3ccc4c(ccc5ccccc54)c3)ccc2N(c2ccc(-c3cccc(-n4c5ccccc5c5ccccc54)c3)cc2)c2cccc(-c3cccc4oc5c6ccccc6ccc5c34)c2)cc1]

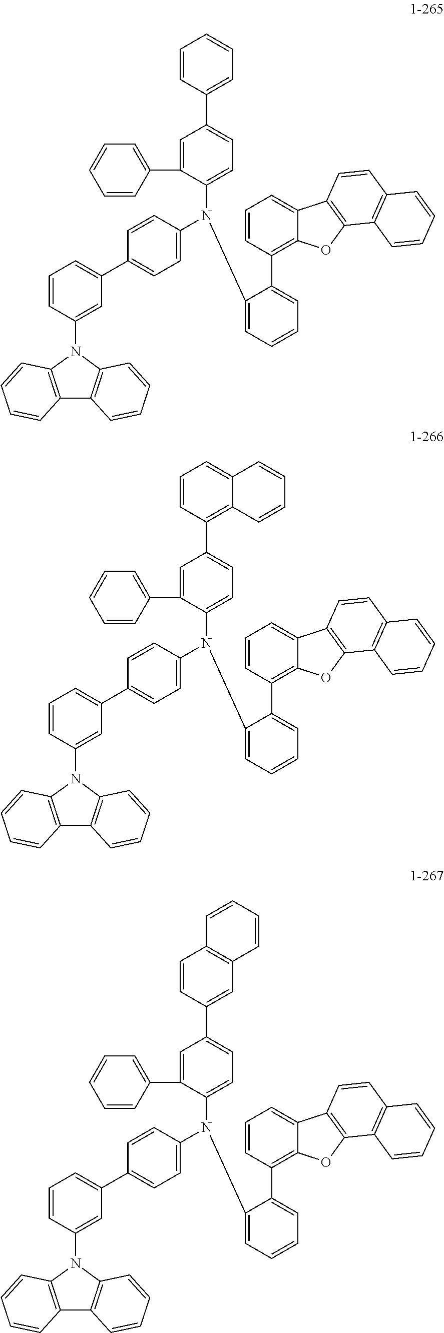 [SMILES: c1ccc(-c2ccc(N(c3ccc(-c4cccc(-n5c6ccccc6c6ccccc65)c4)cc3)c3ccccc3-c3cccc4c3oc3c5ccccc5ccc43)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2ccccc2-c2cccc3c2oc2c4ccccc4ccc32)cc1 ; c1ccc(-c2cc(-c3ccc4ccccc4c3)ccc2N(c2ccc(-c3cccc(-n4c5ccccc5c5ccccc54)c3)cc2)c2ccccc2-c2cccc3c2oc2c4ccccc4ccc32)cc1]

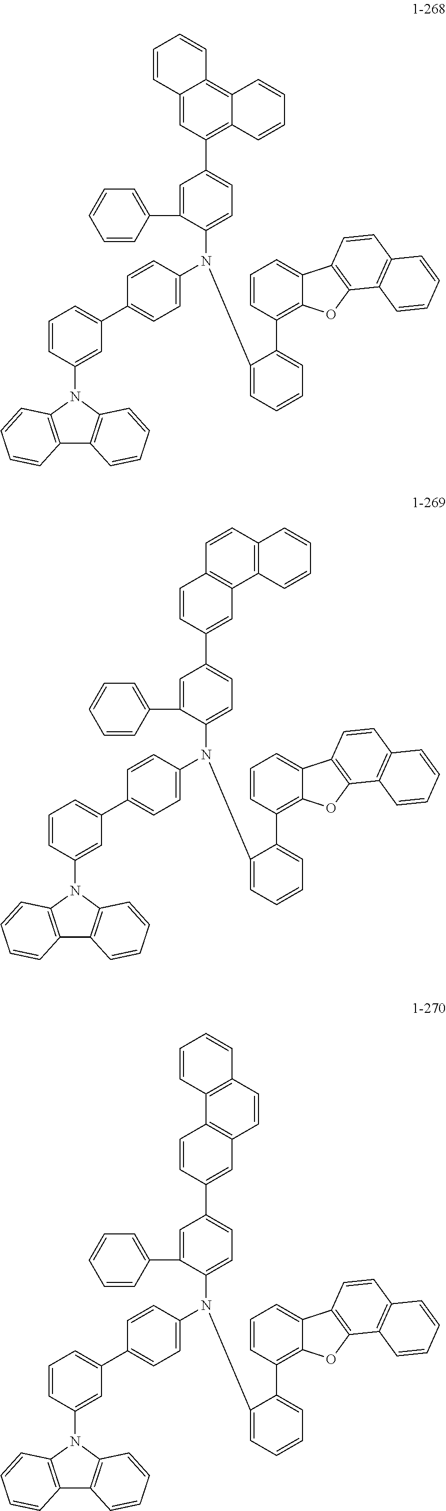 [SMILES: c1ccc(-c2cc(-c3cc4ccccc4c4ccccc34)ccc2N(c2ccc(-c3cccc(-n4c5ccccc5c5ccccc54)c3)cc2)c2ccccc2-c2cccc3c2oc2c4ccccc4ccc32)cc1 ; c1ccc(-c2cc(-c3ccc4ccc5ccccc5c4c3)ccc2N(c2ccc(-c3cccc(-n4c5ccccc5c5ccccc54)c3)cc2)c2ccccc2-c2cccc3c2oc2c4ccccc4ccc32)cc1 ; c1ccc(-c2cc(-c3ccc4c(ccc5ccccc54)c3)ccc2N(c2ccc(-c3cccc(-n4c5ccccc5c5ccccc54)c3)cc2)c2ccccc2-c2cccc3c2oc2c4ccccc4ccc32)cc1]

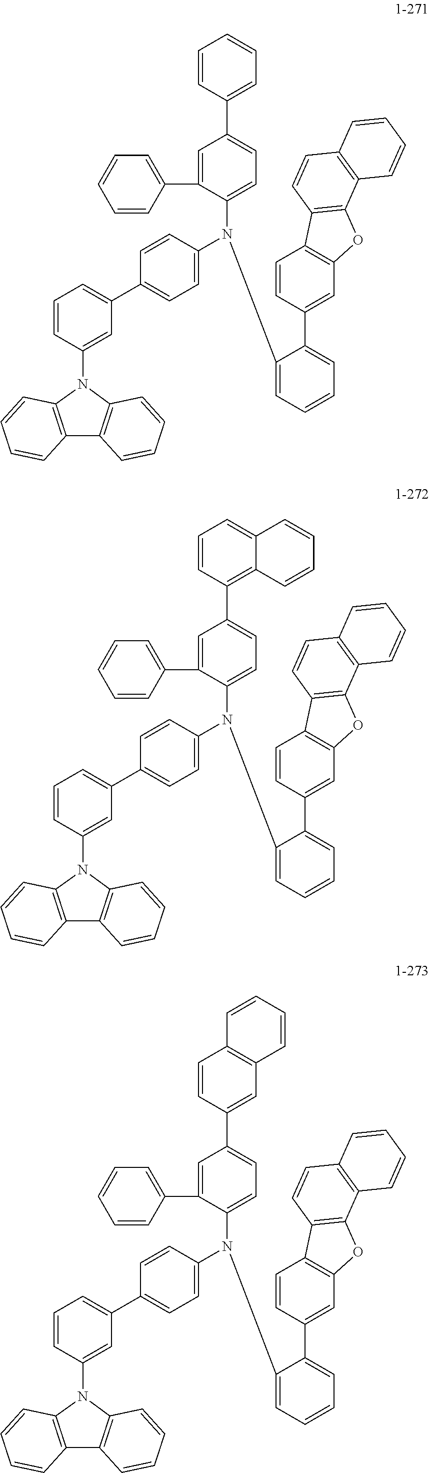 [SMILES: c1ccc(-c2ccc(N(c3ccc(-c4cccc(-n5c6ccccc6c6ccccc65)c4)cc3)c3ccccc3-c3ccc4c(c3)oc3c5ccccc5ccc43)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2ccccc2-c2ccc3c(c2)oc2c4ccccc4ccc32)cc1 ; c1ccc(-c2cc(-c3ccc4ccccc4c3)ccc2N(c2ccc(-c3cccc(-n4c5ccccc5c5ccccc54)c3)cc2)c2ccccc2-c2ccc3c(c2)oc2c4ccccc4ccc32)cc1]

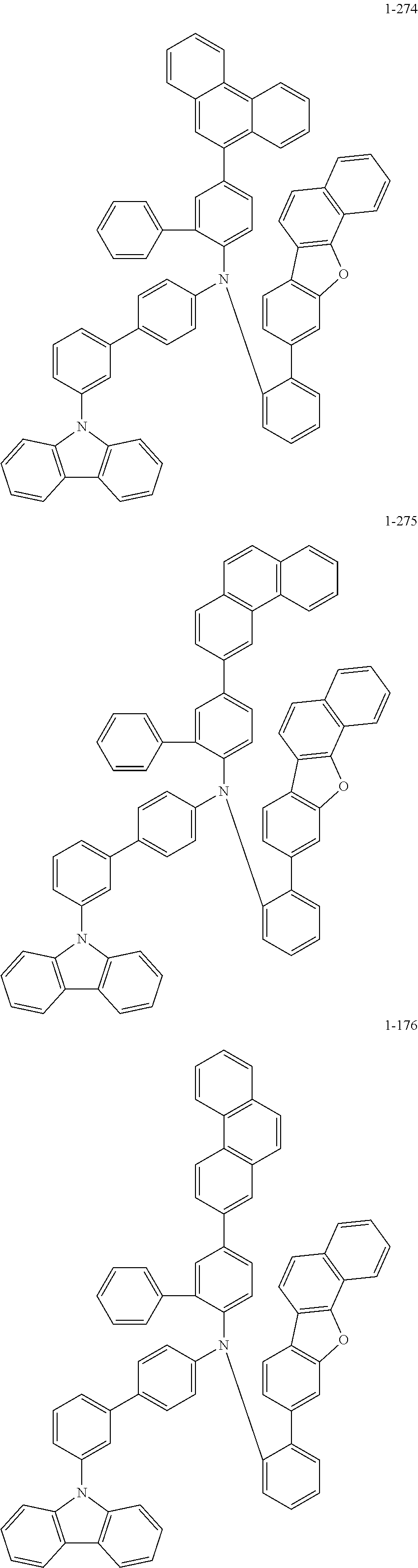 [SMILES: c1ccc(-c2cc(-c3cc4ccccc4c4ccccc34)ccc2N(c2ccc(-c3cccc(-n4c5ccccc5c5ccccc54)c3)cc2)c2ccccc2-c2ccc3c(c2)oc2c4ccccc4ccc32)cc1 ; c1ccc(-c2cc(-c3ccc4ccc5ccccc5c4c3)ccc2N(c2ccc(-c3cccc(-n4c5ccccc5c5ccccc54)c3)cc2)c2ccccc2-c2ccc3c(c2)oc2c4ccccc4ccc32)cc1 ; c1ccc(-c2cc(-c3ccc4c(ccc5ccccc54)c3)ccc2N(c2ccc(-c3cccc(-n4c5ccccc5c5ccccc54)c3)cc2)c2ccccc2-c2ccc3c(c2)oc2c4ccccc4ccc32)cc1]

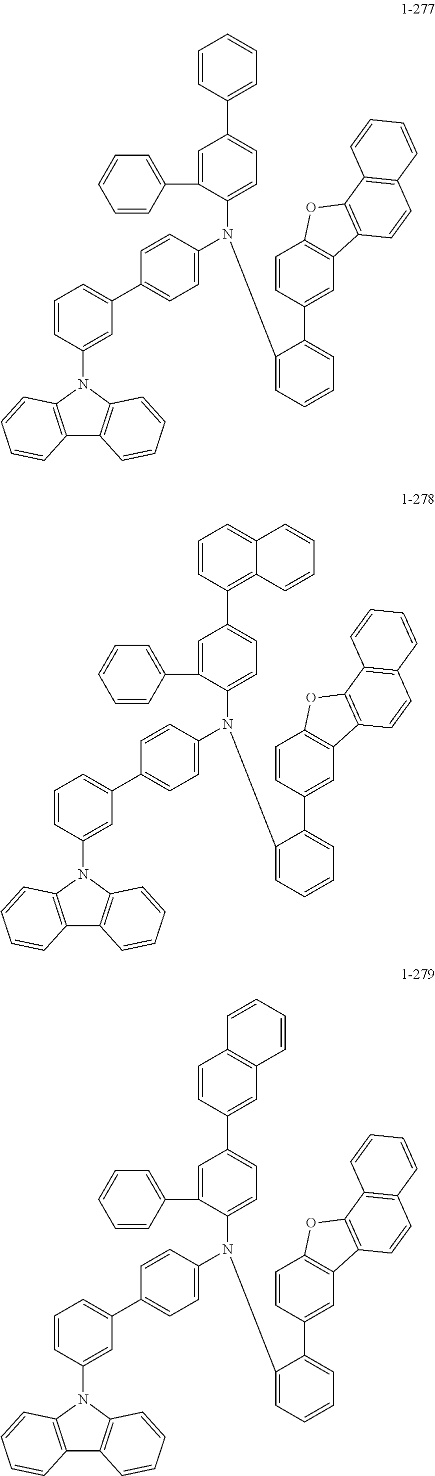 [SMILES: c1ccc(-c2ccc(N(c3ccc(-c4cccc(-n5c6ccccc6c6ccccc65)c4)cc3)c3ccccc3-c3ccc4oc5c6ccccc6ccc5c4c3)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2ccccc2-c2ccc3oc4c5ccccc5ccc4c3c2)cc1 ; c1ccc(-c2cc(-c3ccc4ccccc4c3)ccc2N(c2ccc(-c3cccc(-n4c5ccccc5c5ccccc54)c3)cc2)c2ccccc2-c2ccc3oc4c5ccccc5ccc4c3c2)cc1]

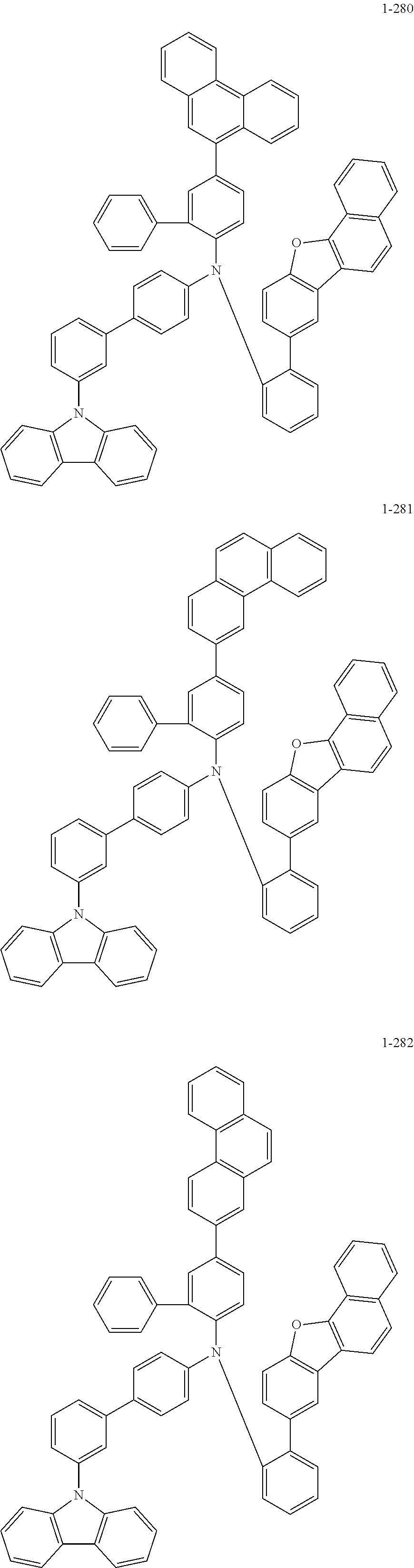 [SMILES: c1ccc(-c2cc(-c3cc4ccccc4c4ccccc34)ccc2N(c2ccc(-c3cccc(-n4c5ccccc5c5ccccc54)c3)cc2)c2ccccc2-c2ccc3oc4c5ccccc5ccc4c3c2)cc1 ; c1ccc(-c2cc(-c3ccc4ccc5ccccc5c4c3)ccc2N(c2ccc(-c3cccc(-n4c5ccccc5c5ccccc54)c3)cc2)c2ccccc2-c2ccc3oc4c5ccccc5ccc4c3c2)cc1 ; c1ccc(-c2cc(-c3ccc4c(ccc5ccccc54)c3)ccc2N(c2ccc(-c3cccc(-n4c5ccccc5c5ccccc54)c3)cc2)c2ccccc2-c2ccc3oc4c5ccccc5ccc4c3c2)cc1]

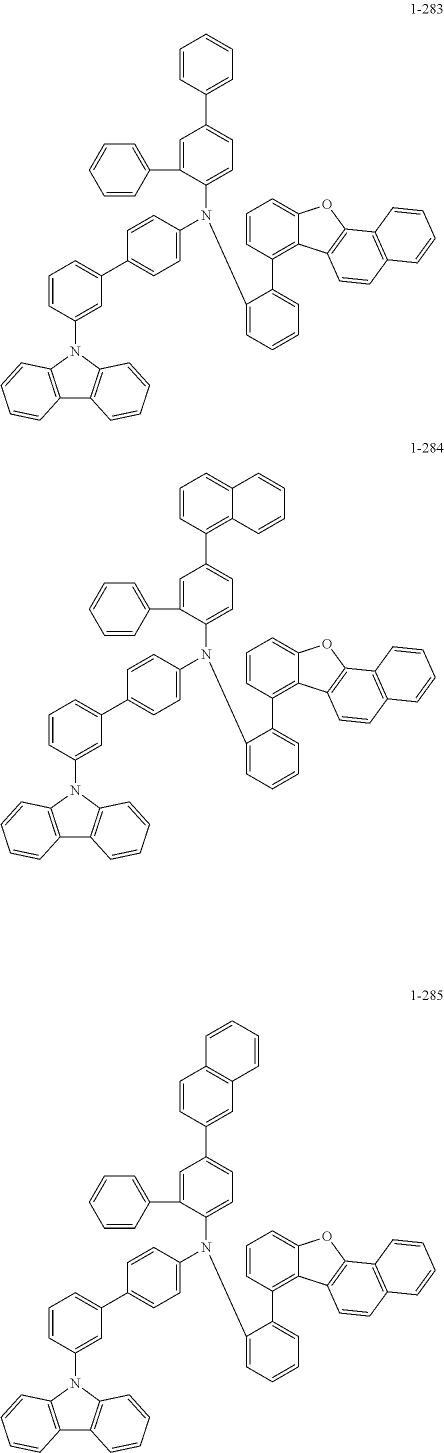 [SMILES: c1ccc(-c2ccc(N(c3ccc(-c4cccc(-n5c6ccccc6c6ccccc65)c4)cc3)c3ccccc3-c3cccc4oc5c6ccccc6ccc5c34)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2ccccc2-c2cccc3oc4c5ccccc5ccc4c23)cc1 ; c1ccc(-c2cc(-c3ccc4ccccc4c3)ccc2N(c2ccc(-c3cccc(-n4c5ccccc5c5ccccc54)c3)cc2)c2ccccc2-c2cccc3oc4c5ccccc5ccc4c23)cc1]

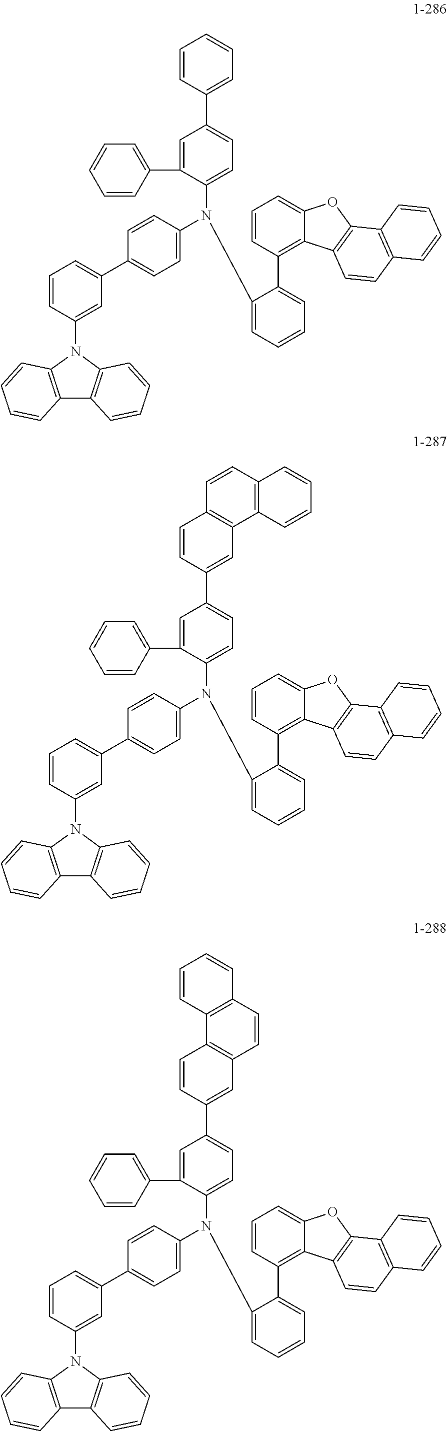 [SMILES: c1ccc(-c2ccc(N(c3ccc(-c4cccc(-n5c6ccccc6c6ccccc65)c4)cc3)c3ccccc3-c3cccc4oc5c6ccccc6ccc5c34)c(-c3ccccc3)c2)cc1 ; c1ccc(-c2cc(-c3ccc4ccc5ccccc5c4c3)ccc2N(c2ccc(-c3cccc(-n4c5ccccc5c5ccccc54)c3)cc2)c2ccccc2-c2cccc3oc4c5ccccc5ccc4c23)cc1 ; c1ccc(-c2cc(-c3ccc4c(ccc5ccccc54)c3)ccc2N(c2ccc(-c3cccc(-n4c5ccccc5c5ccccc54)c3)cc2)c2ccccc2-c2cccc3oc4c5ccccc5ccc4c23)cc1]

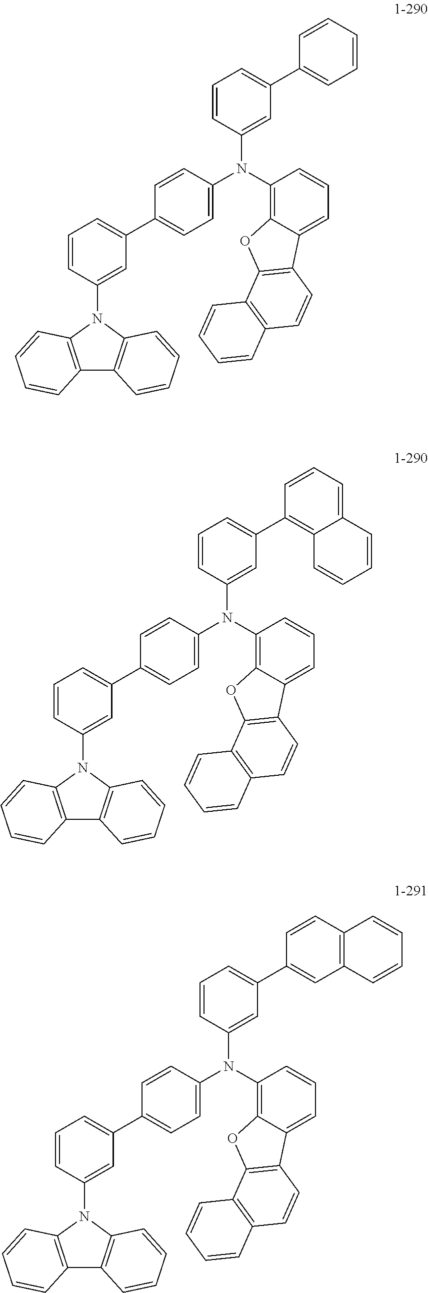 [SMILES: c1ccc(-c2cccc(N(c3ccc(-c4cccc(-n5c6ccccc6c6ccccc65)c4)cc3)c3cccc4c3oc3c5ccccc5ccc43)c2)cc1 ; c1cc(-c2cccc3ccccc23)cc(N(c2ccc(-c3cccc(-n4c5ccccc5c5ccccc54)c3)cc2)c2cccc3c2oc2c4ccccc4ccc32)c1 ; c1cc(-c2ccc3ccccc3c2)cc(N(c2ccc(-c3cccc(-n4c5ccccc5c5ccccc54)c3)cc2)c2cccc3c2oc2c4ccccc4ccc32)c1]

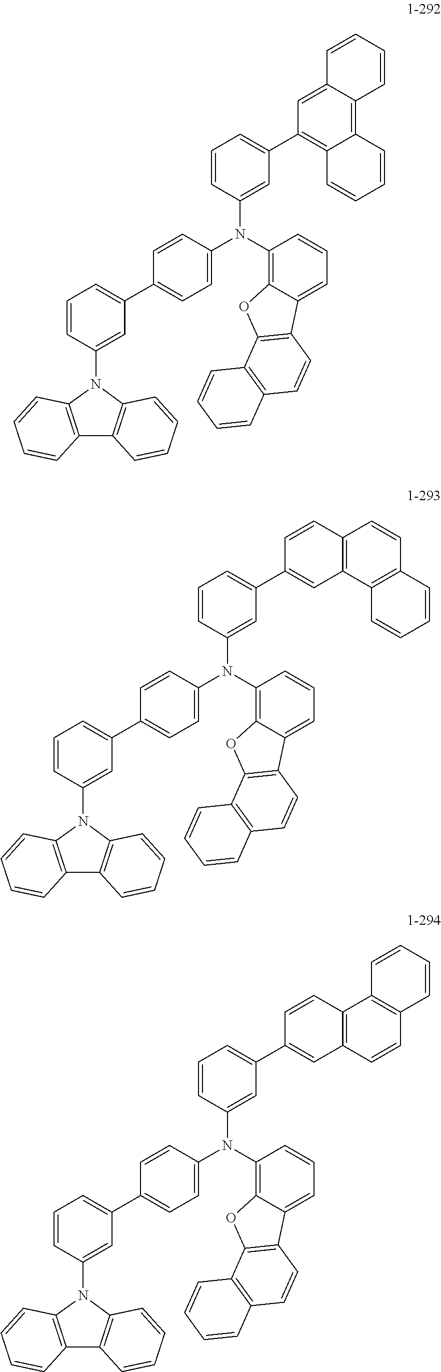 [SMILES: c1cc(-c2cc3ccccc3c3ccccc23)cc(N(c2ccc(-c3cccc(-n4c5ccccc5c5ccccc54)c3)cc2)c2cccc3c2oc2c4ccccc4ccc32)c1 ; c1cc(-c2ccc3ccc4ccccc4c3c2)cc(N(c2ccc(-c3cccc(-n4c5ccccc5c5ccccc54)c3)cc2)c2cccc3c2oc2c4ccccc4ccc32)c1 ; c1cc(-c2ccc3c(ccc4ccccc43)c2)cc(N(c2ccc(-c3cccc(-n4c5ccccc5c5ccccc54)c3)cc2)c2cccc3c2oc2c4ccccc4ccc32)c1]

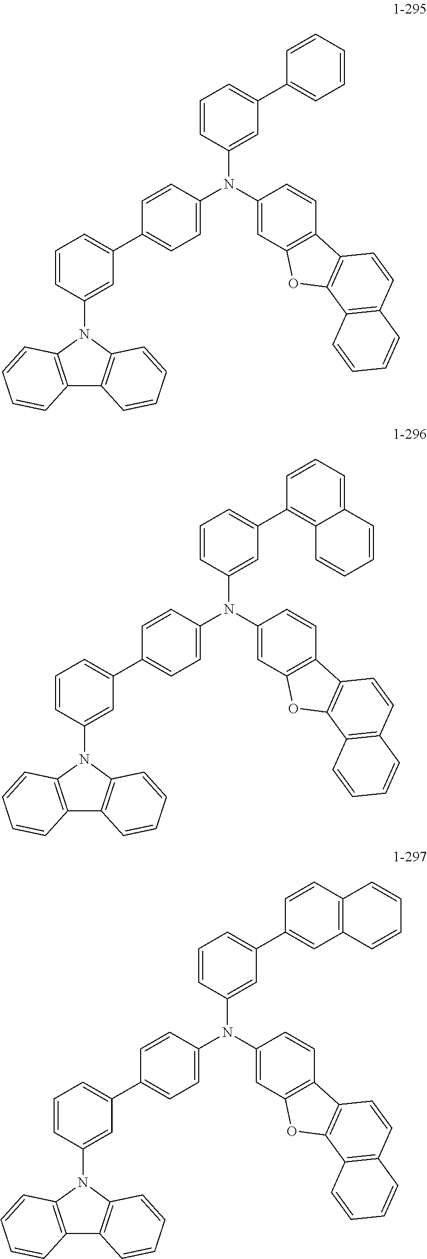 [SMILES: c1ccc(-c2cccc(N(c3ccc(-c4cccc(-n5c6ccccc6c6ccccc65)c4)cc3)c3ccc4c(c3)oc3c5ccccc5ccc43)c2)cc1 ; c1cc(-c2cccc3ccccc23)cc(N(c2ccc(-c3cccc(-n4c5ccccc5c5ccccc54)c3)cc2)c2ccc3c(c2)oc2c4ccccc4ccc32)c1 ; c1cc(-c2ccc3ccccc3c2)cc(N(c2ccc(-c3cccc(-n4c5ccccc5c5ccccc54)c3)cc2)c2ccc3c(c2)oc2c4ccccc4ccc32)c1]

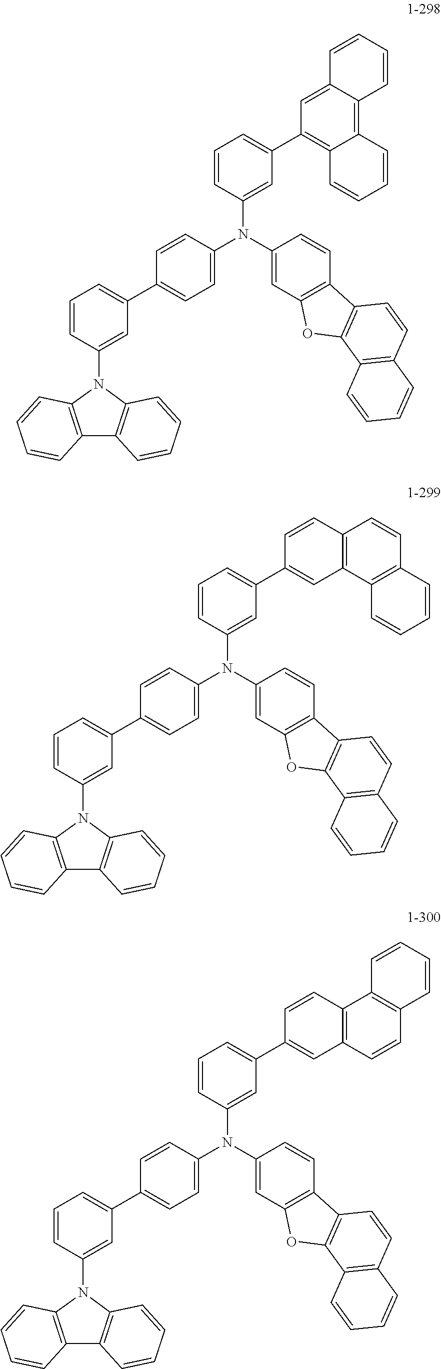 [SMILES: c1cc(-c2cc3ccccc3c3ccccc23)cc(N(c2ccc(-c3cccc(-n4c5ccccc5c5ccccc54)c3)cc2)c2ccc3c(c2)oc2c4ccccc4ccc32)c1 ; c1cc(-c2ccc3ccc4ccccc4c3c2)cc(N(c2ccc(-c3cccc(-n4c5ccccc5c5ccccc54)c3)cc2)c2ccc3c(c2)oc2c4ccccc4ccc32)c1 ; c1cc(-c2ccc3c(ccc4ccccc43)c2)cc(N(c2ccc(-c3cccc(-n4c5ccccc5c5ccccc54)c3)cc2)c2ccc3c(c2)oc2c4ccccc4ccc32)c1]

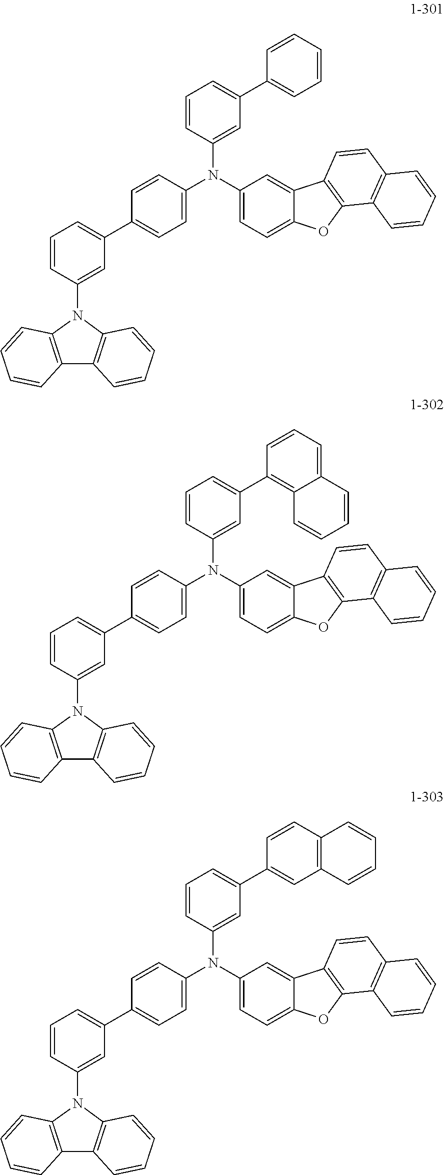 [SMILES: c1ccc(-c2cccc(N(c3ccc(-c4cccc(-n5c6ccccc6c6ccccc65)c4)cc3)c3ccc4oc5c6ccccc6ccc5c4c3)c2)cc1 ; c1cc(-c2cccc3ccccc23)cc(N(c2ccc(-c3cccc(-n4c5ccccc5c5ccccc54)c3)cc2)c2ccc3oc4c5ccccc5ccc4c3c2)c1 ; c1cc(-c2ccc3ccccc3c2)cc(N(c2ccc(-c3cccc(-n4c5ccccc5c5ccccc54)c3)cc2)c2ccc3oc4c5ccccc5ccc4c3c2)c1]

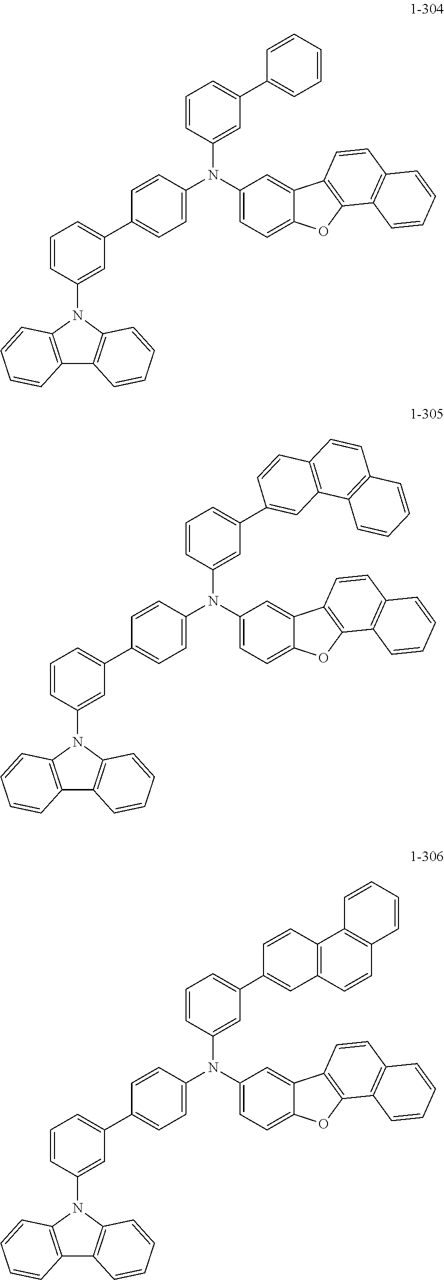 [SMILES: c1ccc(-c2cccc(N(c3ccc(-c4cccc(-n5c6ccccc6c6ccccc65)c4)cc3)c3ccc4oc5c6ccccc6ccc5c4c3)c2)cc1 ; c1cc(-c2ccc3ccc4ccccc4c3c2)cc(N(c2ccc(-c3cccc(-n4c5ccccc5c5ccccc54)c3)cc2)c2ccc3oc4c5ccccc5ccc4c3c2)c1 ; c1cc(-c2ccc3c(ccc4ccccc43)c2)cc(N(c2ccc(-c3cccc(-n4c5ccccc5c5ccccc54)c3)cc2)c2ccc3oc4c5ccccc5ccc4c3c2)c1]

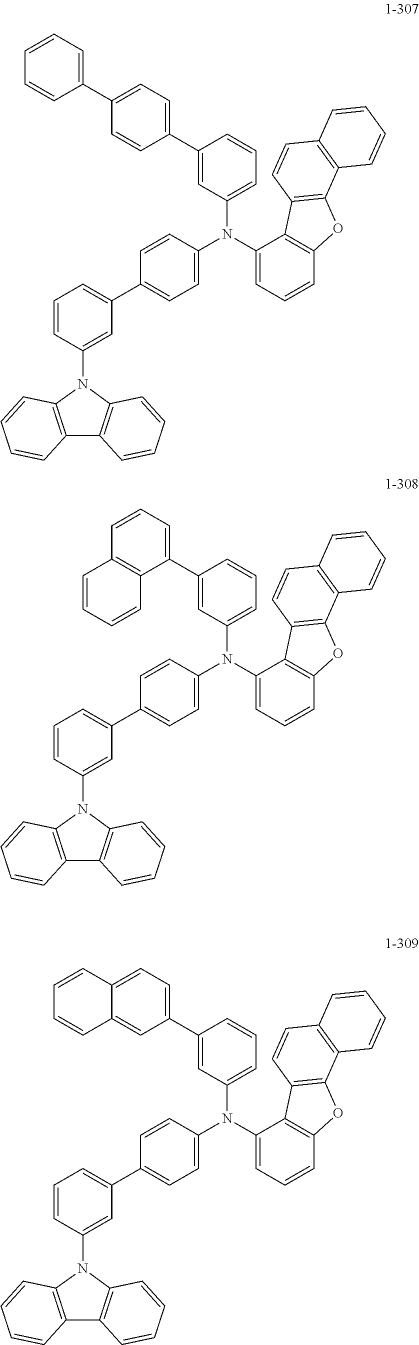 [SMILES: c1ccc(-c2ccc(-c3cccc(N(c4ccc(-c5cccc(-n6c7ccccc7c7ccccc76)c5)cc4)c4cccc5oc6c7ccccc7ccc6c45)c3)cc2)cc1 ; c1cc(-c2cccc3ccccc23)cc(N(c2ccc(-c3cccc(-n4c5ccccc5c5ccccc54)c3)cc2)c2cccc3oc4c5ccccc5ccc4c23)c1 ; c1cc(-c2ccc3ccccc3c2)cc(N(c2ccc(-c3cccc(-n4c5ccccc5c5ccccc54)c3)cc2)c2cccc3oc4c5ccccc5ccc4c23)c1]

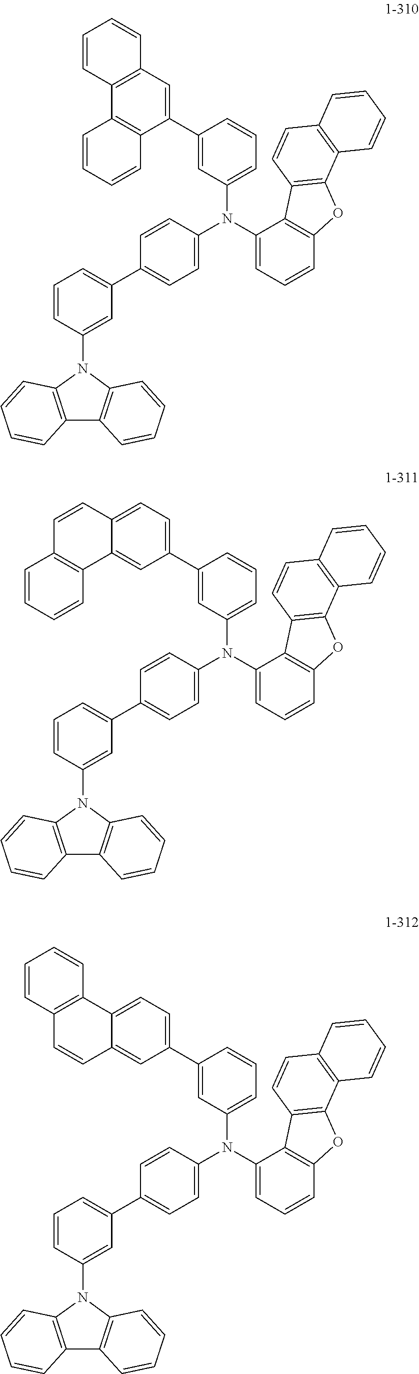 [SMILES: c1cc(-c2cc3ccccc3c3ccccc23)cc(N(c2ccc(-c3cccc(-n4c5ccccc5c5ccccc54)c3)cc2)c2cccc3oc4c5ccccc5ccc4c23)c1 ; c1cc(-c2ccc3ccc4ccccc4c3c2)cc(N(c2ccc(-c3cccc(-n4c5ccccc5c5ccccc54)c3)cc2)c2cccc3oc4c5ccccc5ccc4c23)c1 ; c1cc(-c2ccc3c(ccc4ccccc43)c2)cc(N(c2ccc(-c3cccc(-n4c5ccccc5c5ccccc54)c3)cc2)c2cccc3oc4c5ccccc5ccc4c23)c1]

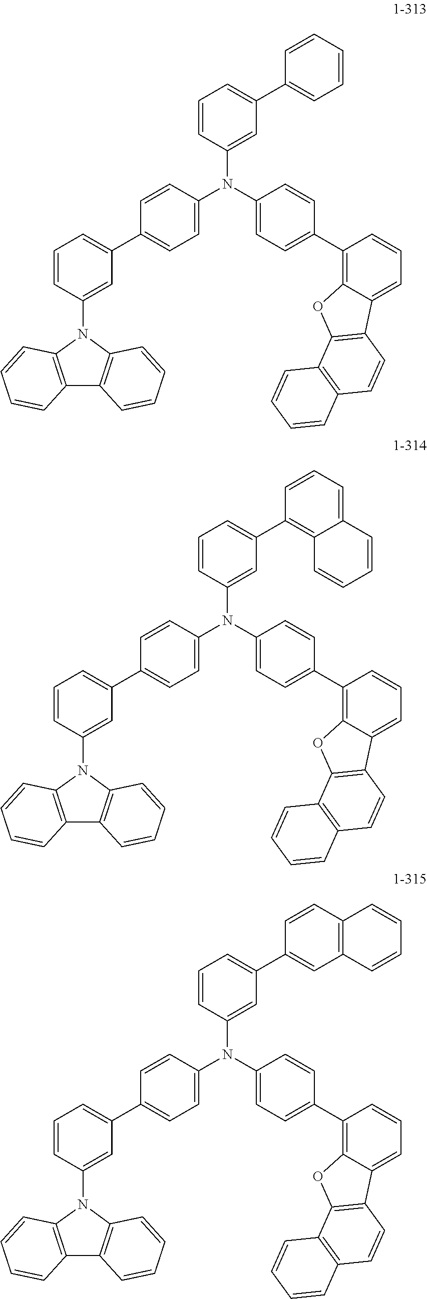

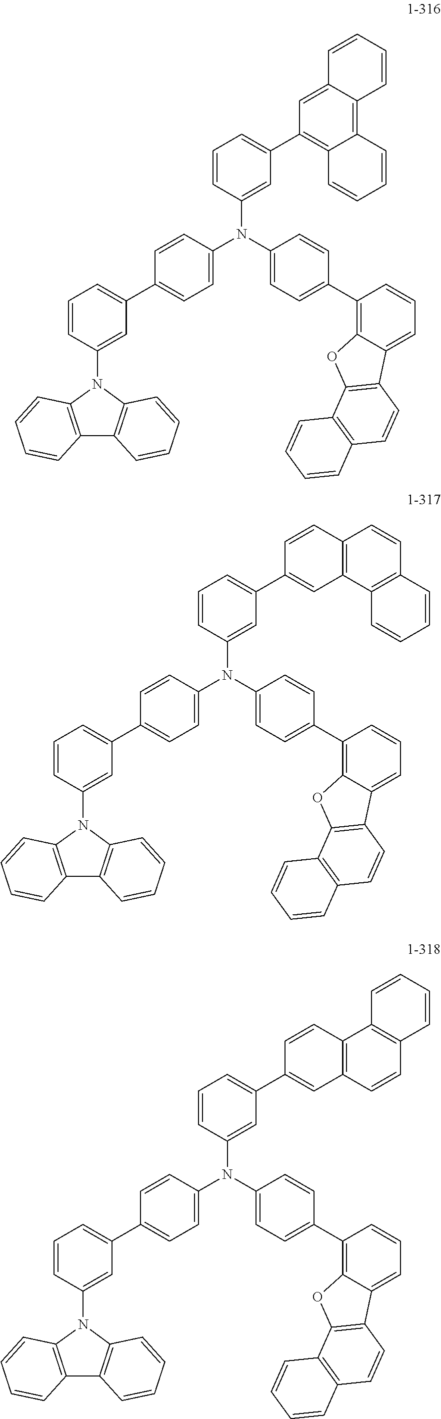 [SMILES: c1cc(-c2cc3ccccc3c3ccccc23)cc(N(c2ccc(-c3cccc(-n4c5ccccc5c5ccccc54)c3)cc2)c2ccc(-c3cccc4c3oc3c5ccccc5ccc43)cc2)c1 ; c1cc(-c2ccc3ccc4ccccc4c3c2)cc(N(c2ccc(-c3cccc(-n4c5ccccc5c5ccccc54)c3)cc2)c2ccc(-c3cccc4c3oc3c5ccccc5ccc43)cc2)c1 ; c1cc(-c2ccc3c(ccc4ccccc43)c2)cc(N(c2ccc(-c3cccc(-n4c5ccccc5c5ccccc54)c3)cc2)c2ccc(-c3cccc4c3oc3c5ccccc5ccc43)cc2)c1]

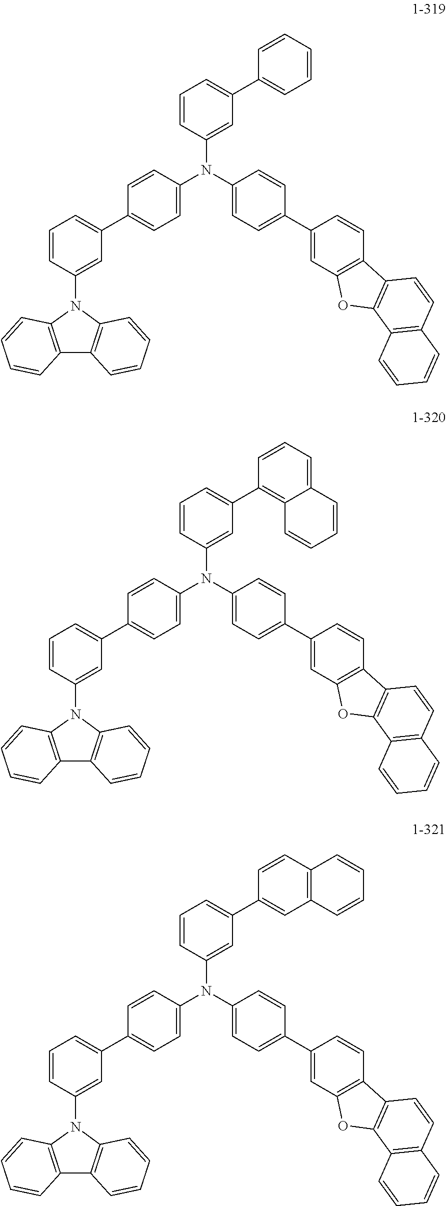 [SMILES: c1ccc(-c2cccc(N(c3ccc(-c4cccc(-n5c6ccccc6c6ccccc65)c4)cc3)c3ccc(-c4ccc5c(c4)oc4c6ccccc6ccc54)cc3)c2)cc1 ; c1cc(-c2cccc3ccccc23)cc(N(c2ccc(-c3cccc(-n4c5ccccc5c5ccccc54)c3)cc2)c2ccc(-c3ccc4c(c3)oc3c5ccccc5ccc43)cc2)c1 ; c1cc(-c2ccc3ccccc3c2)cc(N(c2ccc(-c3cccc(-n4c5ccccc5c5ccccc54)c3)cc2)c2ccc(-c3ccc4c(c3)oc3c5ccccc5ccc43)cc2)c1]

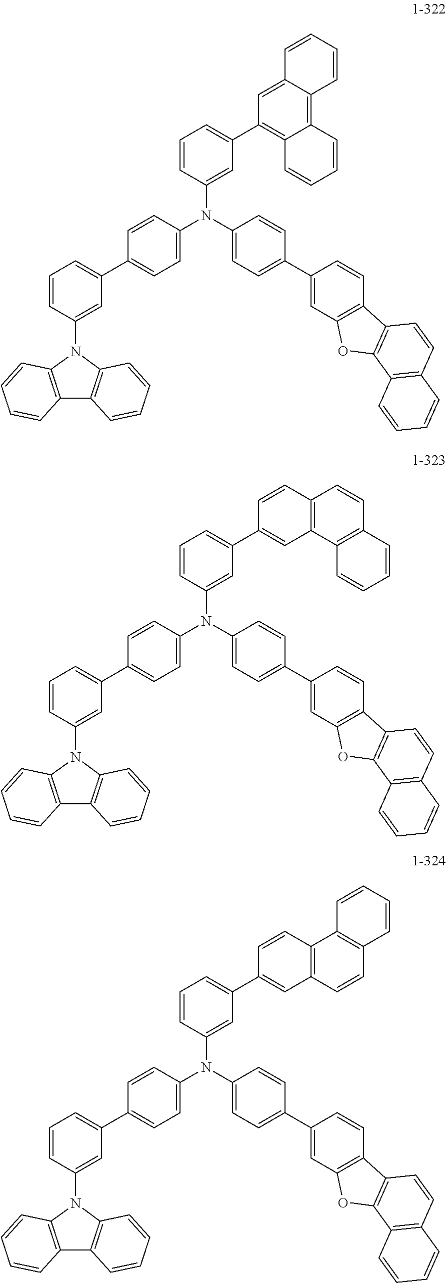 [SMILES: c1cc(-c2cc3ccccc3c3ccccc23)cc(N(c2ccc(-c3cccc(-n4c5ccccc5c5ccccc54)c3)cc2)c2ccc(-c3ccc4c(c3)oc3c5ccccc5ccc43)cc2)c1 ; c1cc(-c2ccc3ccc4ccccc4c3c2)cc(N(c2ccc(-c3cccc(-n4c5ccccc5c5ccccc54)c3)cc2)c2ccc(-c3ccc4c(c3)oc3c5ccccc5ccc43)cc2)c1 ; c1cc(-c2ccc3c(ccc4ccccc43)c2)cc(N(c2ccc(-c3cccc(-n4c5ccccc5c5ccccc54)c3)cc2)c2ccc(-c3ccc4c(c3)oc3c5ccccc5ccc43)cc2)c1]

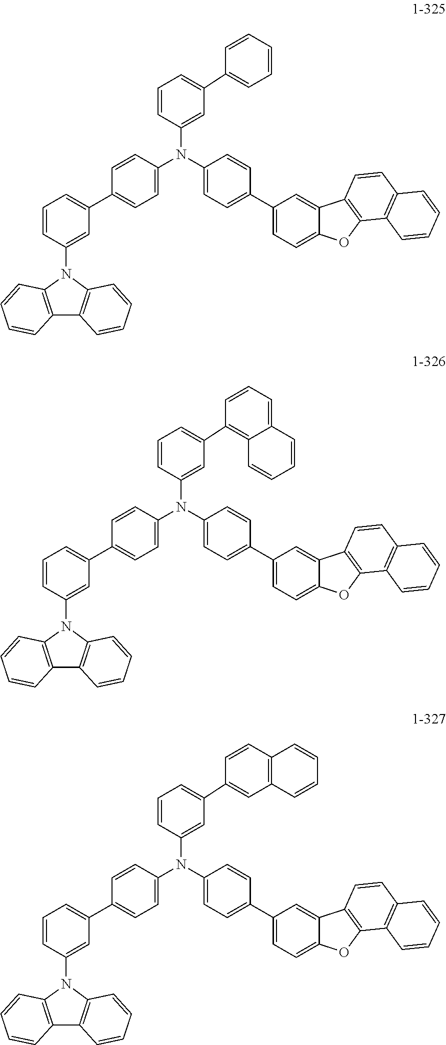 [SMILES: c1ccc(-c2cccc(N(c3ccc(-c4cccc(-n5c6ccccc6c6ccccc65)c4)cc3)c3ccc(-c4ccc5oc6c7ccccc7ccc6c5c4)cc3)c2)cc1 ; c1cc(-c2cccc3ccccc23)cc(N(c2ccc(-c3cccc(-n4c5ccccc5c5ccccc54)c3)cc2)c2ccc(-c3ccc4oc5c6ccccc6ccc5c4c3)cc2)c1 ; c1cc(-c2ccc3ccccc3c2)cc(N(c2ccc(-c3cccc(-n4c5ccccc5c5ccccc54)c3)cc2)c2ccc(-c3ccc4oc5c6ccccc6ccc5c4c3)cc2)c1]

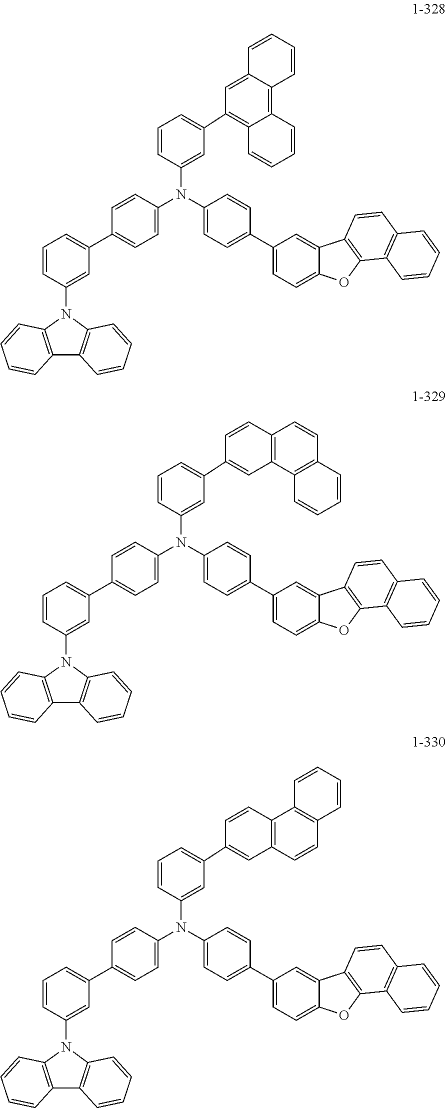 [SMILES: c1cc(-c2cc3ccccc3c3ccccc23)cc(N(c2ccc(-c3cccc(-n4c5ccccc5c5ccccc54)c3)cc2)c2ccc(-c3ccc4oc5c6ccccc6ccc5c4c3)cc2)c1 ; c1cc(-c2ccc3ccc4ccccc4c3c2)cc(N(c2ccc(-c3cccc(-n4c5ccccc5c5ccccc54)c3)cc2)c2ccc(-c3ccc4oc5c6ccccc6ccc5c4c3)cc2)c1 ; c1cc(-c2ccc3c(ccc4ccccc43)c2)cc(N(c2ccc(-c3cccc(-n4c5ccccc5c5ccccc54)c3)cc2)c2ccc(-c3ccc4oc5c6ccccc6ccc5c4c3)cc2)c1]

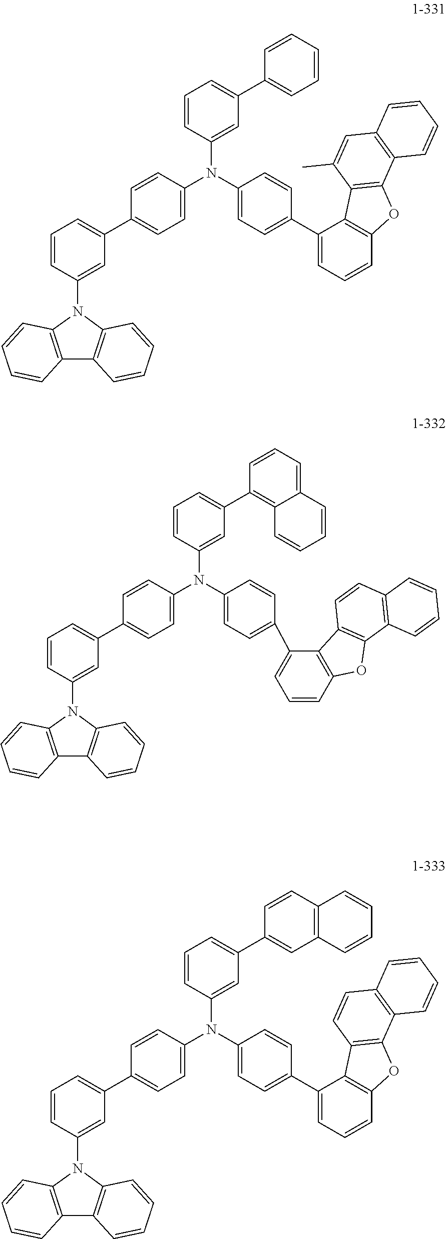 [SMILES: Cc1cc2ccccc2c2oc3cccc(-c4ccc(N(c5ccc(-c6cccc(-n7c8ccccc8c8ccccc87)c6)cc5)c5cccc(-c6ccccc6)c5)cc4)c3c12 ; c1cc(-c2cccc3ccccc23)cc(N(c2ccc(-c3cccc(-n4c5ccccc5c5ccccc54)c3)cc2)c2ccc(-c3cccc4oc5c6ccccc6ccc5c34)cc2)c1 ; c1cc(-c2ccc3ccccc3c2)cc(N(c2ccc(-c3cccc(-n4c5ccccc5c5ccccc54)c3)cc2)c2ccc(-c3cccc4oc5c6ccccc6ccc5c34)cc2)c1]

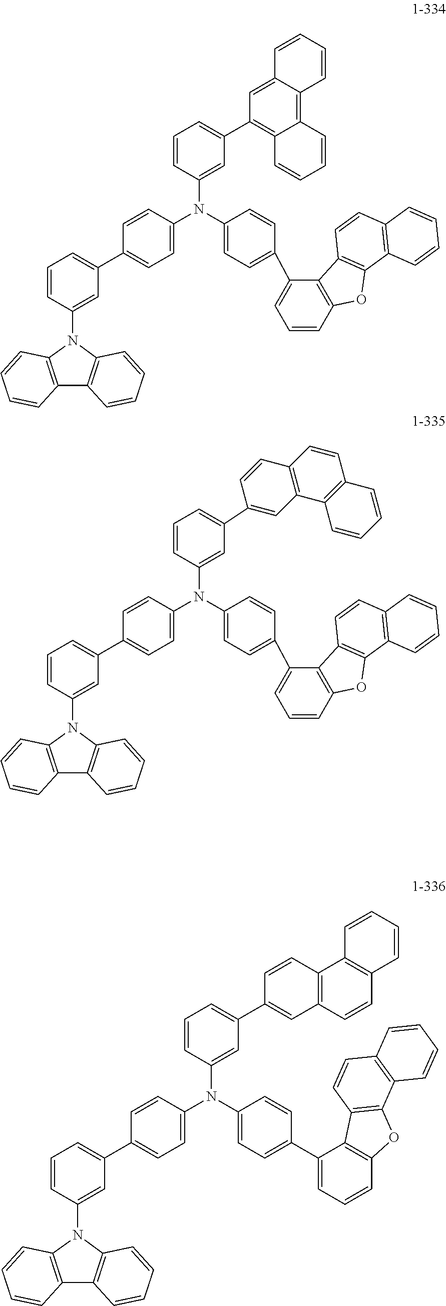 [SMILES: c1cc(-c2cc3ccccc3c3ccccc23)cc(N(c2ccc(-c3cccc(-n4c5ccccc5c5ccccc54)c3)cc2)c2ccc(-c3cccc4oc5c6ccccc6ccc5c34)cc2)c1 ; c1cc(-c2ccc3ccc4ccccc4c3c2)cc(N(c2ccc(-c3cccc(-n4c5ccccc5c5ccccc54)c3)cc2)c2ccc(-c3cccc4oc5c6ccccc6ccc5c34)cc2)c1 ; c1cc(-c2ccc3c(ccc4ccccc43)c2)cc(N(c2ccc(-c3cccc(-n4c5ccccc5c5ccccc54)c3)cc2)c2ccc(-c3cccc4oc5c6ccccc6ccc5c34)cc2)c1]

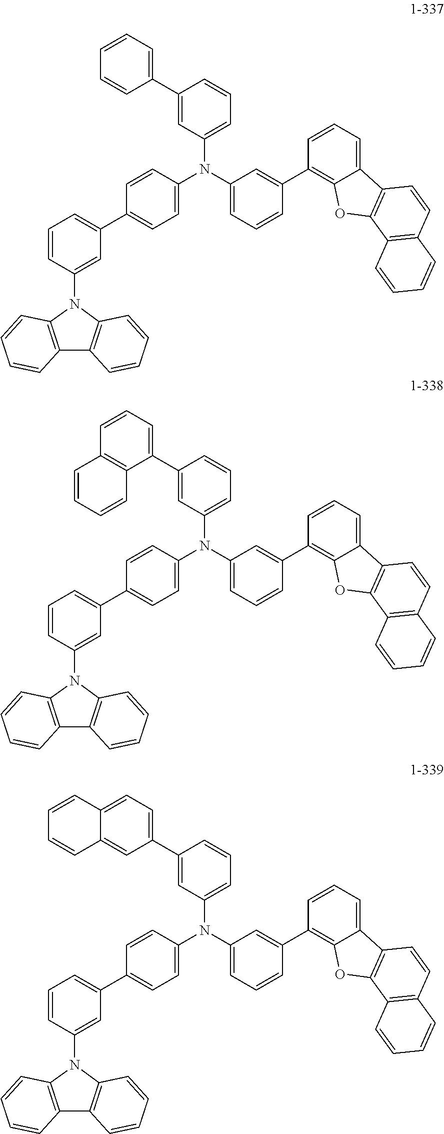

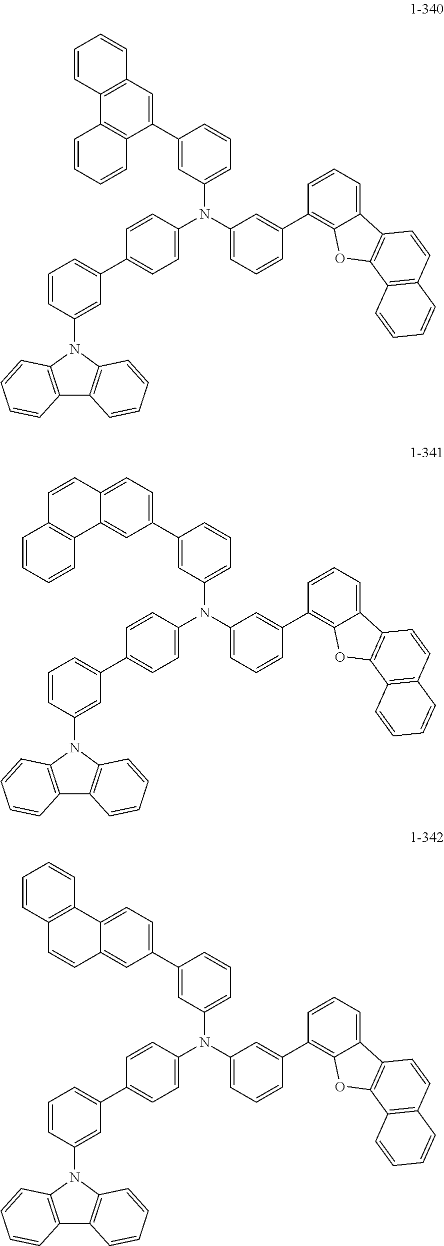 [SMILES: c1cc(-c2cc3ccccc3c3ccccc23)cc(N(c2ccc(-c3cccc(-n4c5ccccc5c5ccccc54)c3)cc2)c2cccc(-c3cccc4c3oc3c5ccccc5ccc43)c2)c1 ; c1cc(-c2ccc3ccc4ccccc4c3c2)cc(N(c2ccc(-c3cccc(-n4c5ccccc5c5ccccc54)c3)cc2)c2cccc(-c3cccc4c3oc3c5ccccc5ccc43)c2)c1 ; c1cc(-c2ccc3c(ccc4ccccc43)c2)cc(N(c2ccc(-c3cccc(-n4c5ccccc5c5ccccc54)c3)cc2)c2cccc(-c3cccc4c3oc3c5ccccc5ccc43)c2)c1]

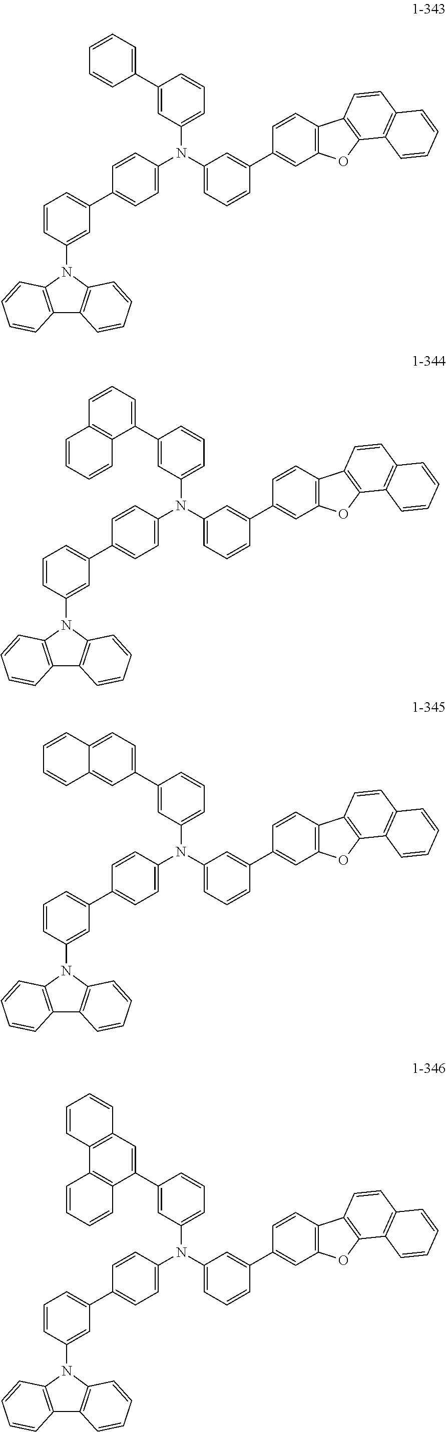 [SMILES: c1ccc(-c2cccc(N(c3ccc(-c4cccc(-n5c6ccccc6c6ccccc65)c4)cc3)c3cccc(-c4ccc5c(c4)oc4c6ccccc6ccc54)c3)c2)cc1 ; c1cc(-c2ccc3c(c2)oc2c4ccccc4ccc32)cc(N(c2ccc(-c3cccc(-n4c5ccccc5c5ccccc54)c3)cc2)c2cccc(-c3cccc4ccccc34)c2)c1 ; c1cc(-c2ccc3ccccc3c2)cc(N(c2ccc(-c3cccc(-n4c5ccccc5c5ccccc54)c3)cc2)c2cccc(-c3ccc4c(c3)oc3c5ccccc5ccc43)c2)c1 ; c1cc(-c2ccc3c(c2)oc2c4ccccc4ccc32)cc(N(c2ccc(-c3cccc(-n4c5ccccc5c5ccccc54)c3)cc2)c2cccc(-c3cc4ccccc4c4ccccc34)c2)c1]

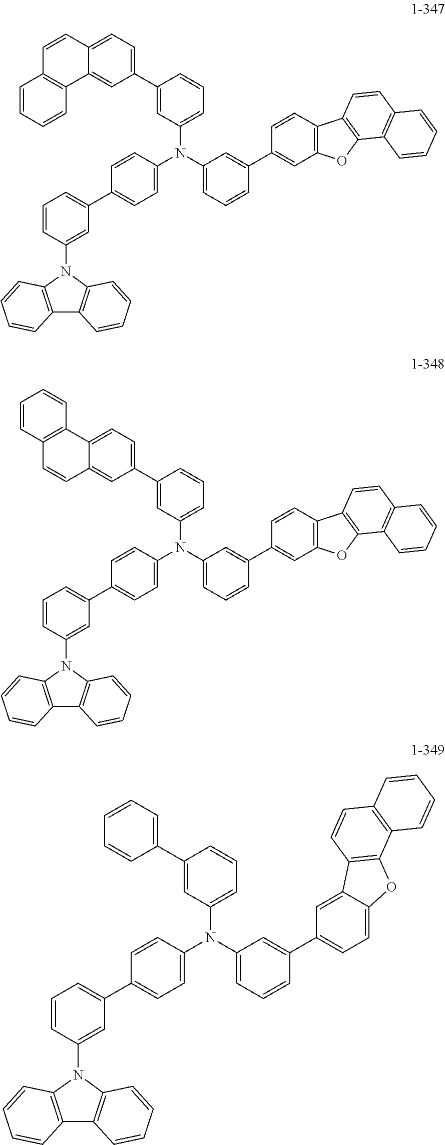 [SMILES: c1cc(-c2ccc3c(c2)oc2c4ccccc4ccc32)cc(N(c2ccc(-c3cccc(-n4c5ccccc5c5ccccc54)c3)cc2)c2cccc(-c3ccc4ccc5ccccc5c4c3)c2)c1 ; c1cc(-c2ccc3c(ccc4ccccc43)c2)cc(N(c2ccc(-c3cccc(-n4c5ccccc5c5ccccc54)c3)cc2)c2cccc(-c3ccc4c(c3)oc3c5ccccc5ccc43)c2)c1 ; c1ccc(-c2cccc(N(c3ccc(-c4cccc(-n5c6ccccc6c6ccccc65)c4)cc3)c3cccc(-c4ccc5oc6c7ccccc7ccc6c5c4)c3)c2)cc1]

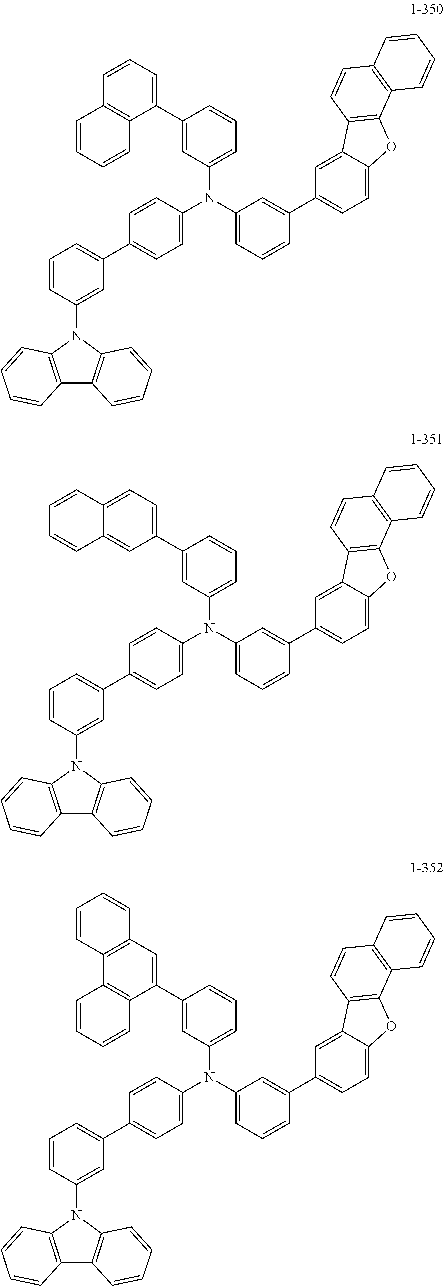 [SMILES: c1cc(-c2ccc3oc4c5ccccc5ccc4c3c2)cc(N(c2ccc(-c3cccc(-n4c5ccccc5c5ccccc54)c3)cc2)c2cccc(-c3cccc4ccccc34)c2)c1 ; c1cc(-c2ccc3ccccc3c2)cc(N(c2ccc(-c3cccc(-n4c5ccccc5c5ccccc54)c3)cc2)c2cccc(-c3ccc4oc5c6ccccc6ccc5c4c3)c2)c1 ; c1cc(-c2ccc3oc4c5ccccc5ccc4c3c2)cc(N(c2ccc(-c3cccc(-n4c5ccccc5c5ccccc54)c3)cc2)c2cccc(-c3cc4ccccc4c4ccccc34)c2)c1]

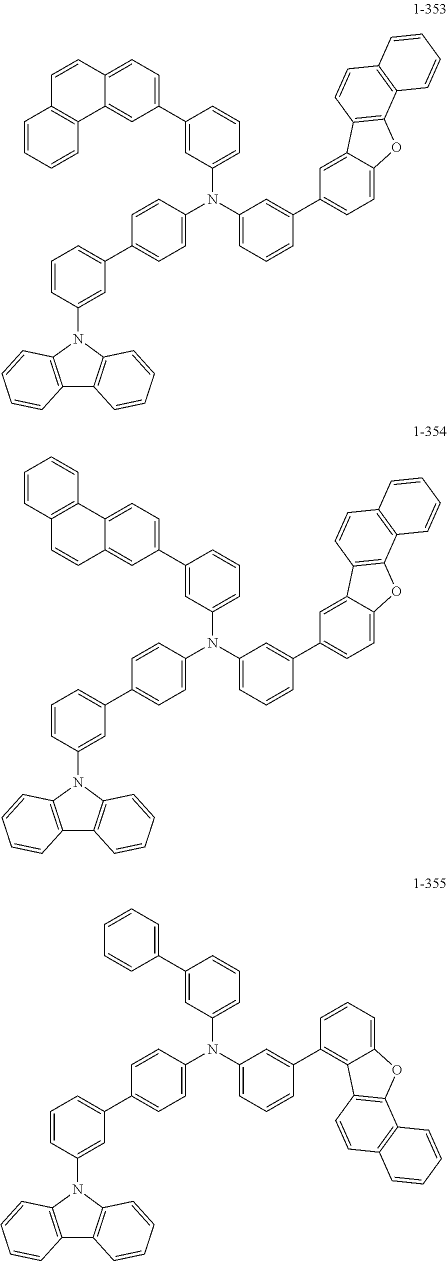 [SMILES: c1cc(-c2ccc3ccc4ccccc4c3c2)cc(N(c2ccc(-c3cccc(-n4c5ccccc5c5ccccc54)c3)cc2)c2cccc(-c3ccc4oc5c6ccccc6ccc5c4c3)c2)c1 ; c1cc(-c2ccc3c(ccc4ccccc43)c2)cc(N(c2ccc(-c3cccc(-n4c5ccccc5c5ccccc54)c3)cc2)c2cccc(-c3ccc4oc5c6ccccc6ccc5c4c3)c2)c1 ; c1ccc(-c2cccc(N(c3ccc(-c4cccc(-n5c6ccccc6c6ccccc65)c4)cc3)c3cccc(-c4cccc5oc6c7ccccc7ccc6c45)c3)c2)cc1]

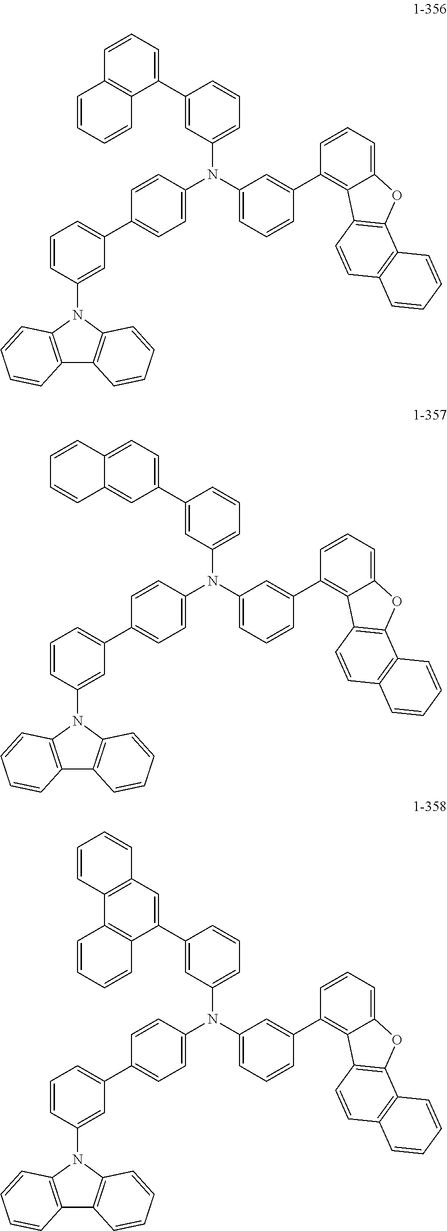

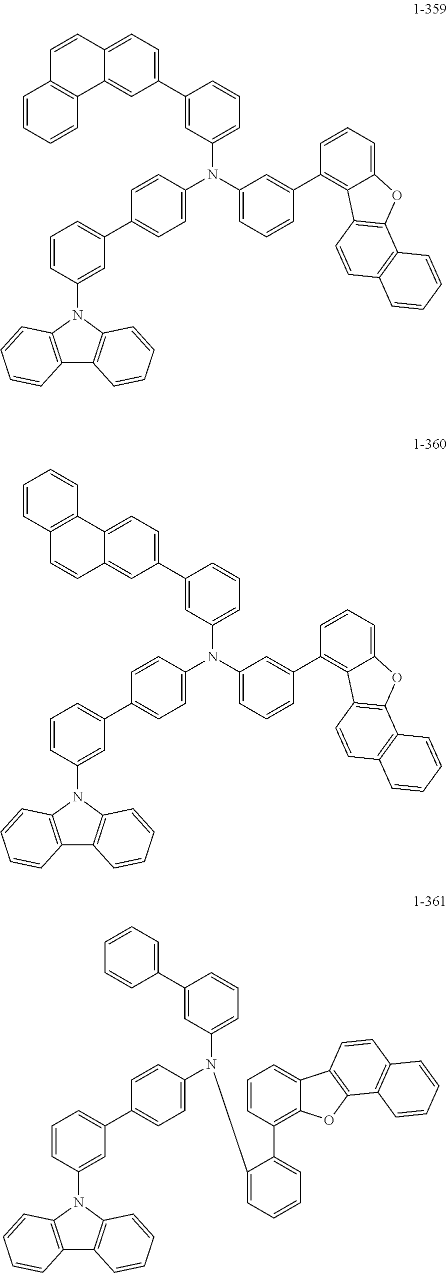 [SMILES: c1cc(-c2ccc3ccc4ccccc4c3c2)cc(N(c2ccc(-c3cccc(-n4c5ccccc5c5ccccc54)c3)cc2)c2cccc(-c3cccc4oc5c6ccccc6ccc5c34)c2)c1 ; c1cc(-c2ccc3c(ccc4ccccc43)c2)cc(N(c2ccc(-c3cccc(-n4c5ccccc5c5ccccc54)c3)cc2)c2cccc(-c3cccc4oc5c6ccccc6ccc5c34)c2)c1 ; c1ccc(-c2cccc(N(c3ccc(-c4cccc(-n5c6ccccc6c6ccccc65)c4)cc3)c3ccccc3-c3cccc4c3oc3c5ccccc5ccc43)c2)cc1]

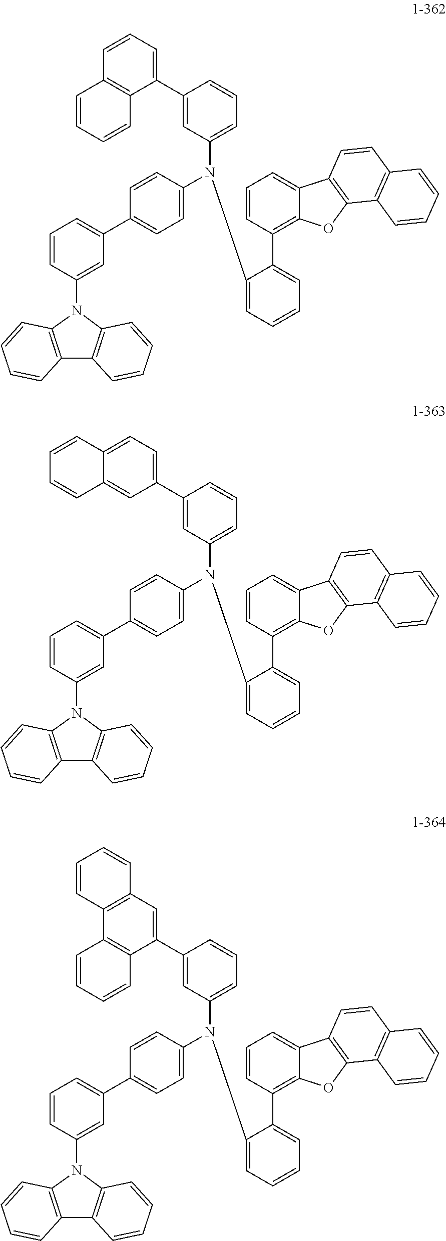 [SMILES: c1cc(-c2cccc3ccccc23)cc(N(c2ccc(-c3cccc(-n4c5ccccc5c5ccccc54)c3)cc2)c2ccccc2-c2cccc3c2oc2c4ccccc4ccc32)c1 ; c1cc(-c2ccc3ccccc3c2)cc(N(c2ccc(-c3cccc(-n4c5ccccc5c5ccccc54)c3)cc2)c2ccccc2-c2cccc3c2oc2c4ccccc4ccc32)c1 ; c1cc(-c2cc3ccccc3c3ccccc23)cc(N(c2ccc(-c3cccc(-n4c5ccccc5c5ccccc54)c3)cc2)c2ccccc2-c2cccc3c2oc2c4ccccc4ccc32)c1]

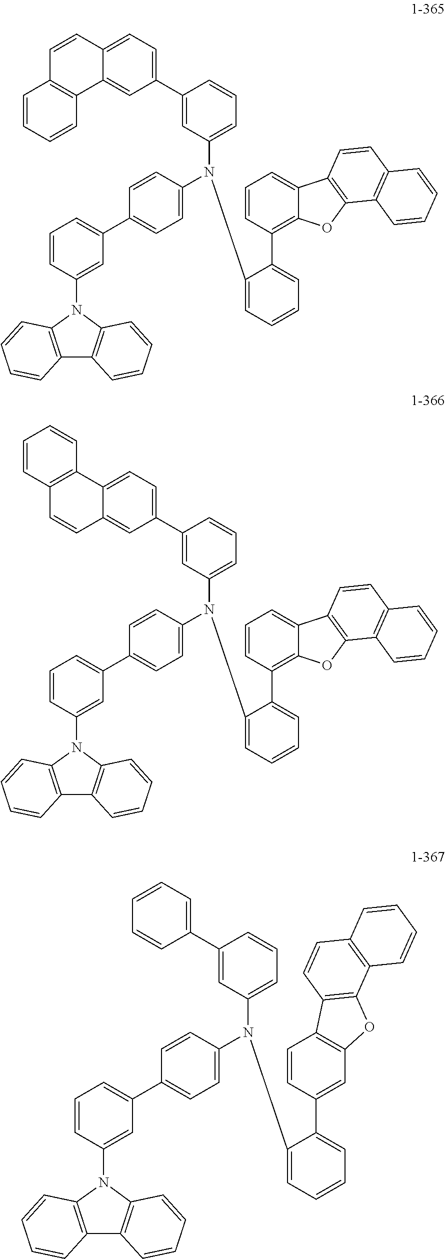 [SMILES: c1cc(-c2ccc3ccc4ccccc4c3c2)cc(N(c2ccc(-c3cccc(-n4c5ccccc5c5ccccc54)c3)cc2)c2ccccc2-c2cccc3c2oc2c4ccccc4ccc32)c1 ; c1cc(-c2ccc3c(ccc4ccccc43)c2)cc(N(c2ccc(-c3cccc(-n4c5ccccc5c5ccccc54)c3)cc2)c2ccccc2-c2cccc3c2oc2c4ccccc4ccc32)c1 ; c1ccc(-c2cccc(N(c3ccc(-c4cccc(-n5c6ccccc6c6ccccc65)c4)cc3)c3ccccc3-c3ccc4c(c3)oc3c5ccccc5ccc43)c2)cc1]

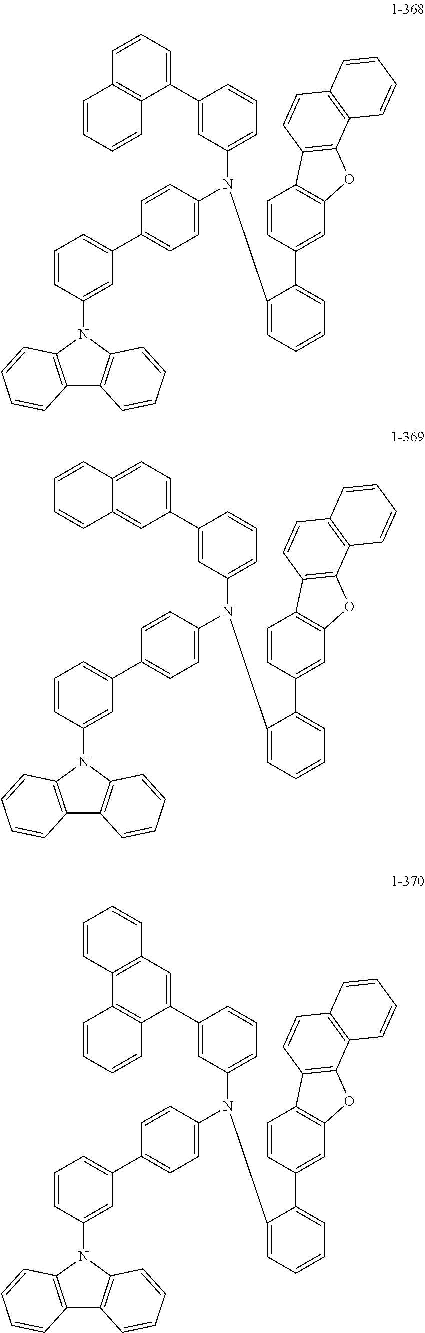 [SMILES: c1cc(-c2cccc3ccccc23)cc(N(c2ccc(-c3cccc(-n4c5ccccc5c5ccccc54)c3)cc2)c2ccccc2-c2ccc3c(c2)oc2c4ccccc4ccc32)c1 ; c1cc(-c2ccc3ccccc3c2)cc(N(c2ccc(-c3cccc(-n4c5ccccc5c5ccccc54)c3)cc2)c2ccccc2-c2ccc3c(c2)oc2c4ccccc4ccc32)c1 ; c1cc(-c2cc3ccccc3c3ccccc23)cc(N(c2ccc(-c3cccc(-n4c5ccccc5c5ccccc54)c3)cc2)c2ccccc2-c2ccc3c(c2)oc2c4ccccc4ccc32)c1]

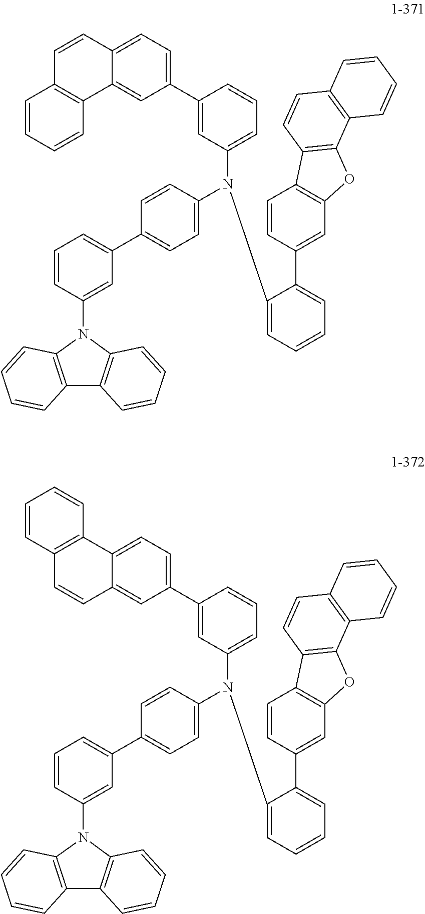 [SMILES: c1cc(-c2ccc3ccc4ccccc4c3c2)cc(N(c2ccc(-c3cccc(-n4c5ccccc5c5ccccc54)c3)cc2)c2ccccc2-c2ccc3c(c2)oc2c4ccccc4ccc32)c1 ; c1cc(-c2ccc3c(ccc4ccccc43)c2)cc(N(c2ccc(-c3cccc(-n4c5ccccc5c5ccccc54)c3)cc2)c2ccccc2-c2ccc3c(c2)oc2c4ccccc4ccc32)c1]

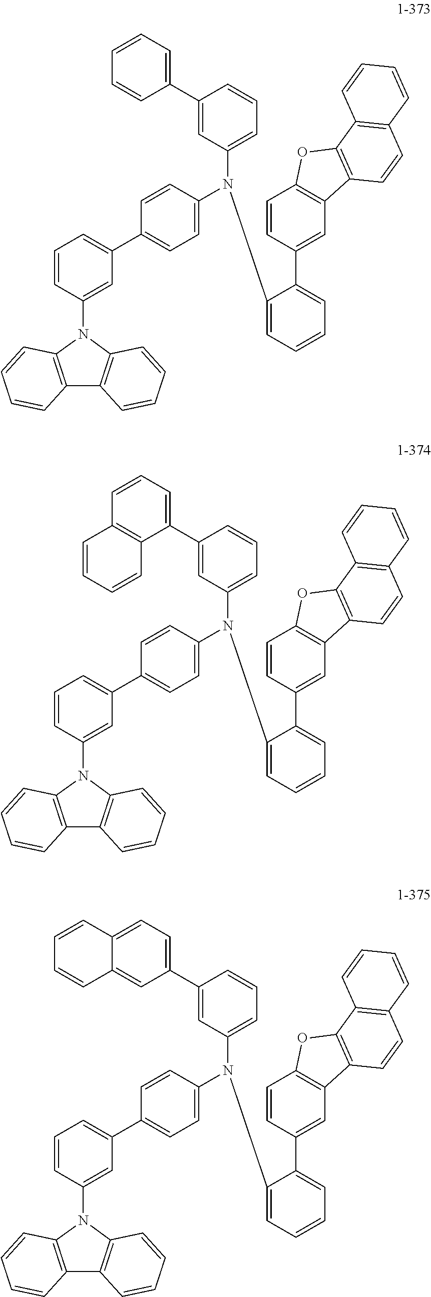 [SMILES: c1ccc(-c2cccc(N(c3ccc(-c4cccc(-n5c6ccccc6c6ccccc65)c4)cc3)c3ccccc3-c3ccc4oc5c6ccccc6ccc5c4c3)c2)cc1 ; c1cc(-c2cccc3ccccc23)cc(N(c2ccc(-c3cccc(-n4c5ccccc5c5ccccc54)c3)cc2)c2ccccc2-c2ccc3oc4c5ccccc5ccc4c3c2)c1 ; c1cc(-c2ccc3ccccc3c2)cc(N(c2ccc(-c3cccc(-n4c5ccccc5c5ccccc54)c3)cc2)c2ccccc2-c2ccc3oc4c5ccccc5ccc4c3c2)c1]

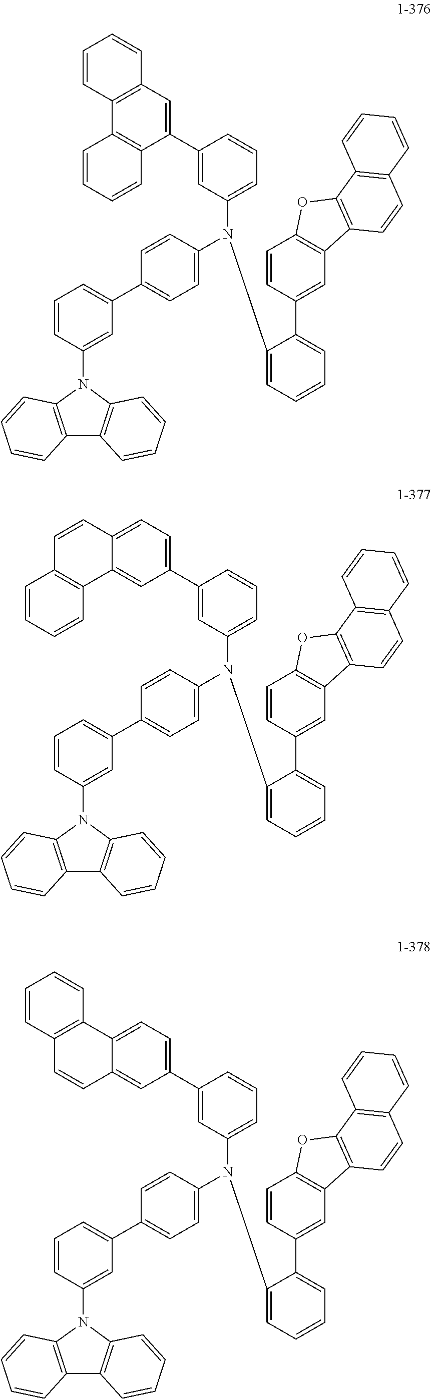 [SMILES: c1cc(-c2cc3ccccc3c3ccccc23)cc(N(c2ccc(-c3cccc(-n4c5ccccc5c5ccccc54)c3)cc2)c2ccccc2-c2ccc3oc4c5ccccc5ccc4c3c2)c1 ; c1cc(-c2ccc3ccc4ccccc4c3c2)cc(N(c2ccc(-c3cccc(-n4c5ccccc5c5ccccc54)c3)cc2)c2ccccc2-c2ccc3oc4c5ccccc5ccc4c3c2)c1 ; c1cc(-c2ccc3c(ccc4ccccc43)c2)cc(N(c2ccc(-c3cccc(-n4c5ccccc5c5ccccc54)c3)cc2)c2ccccc2-c2ccc3oc4c5ccccc5ccc4c3c2)c1]

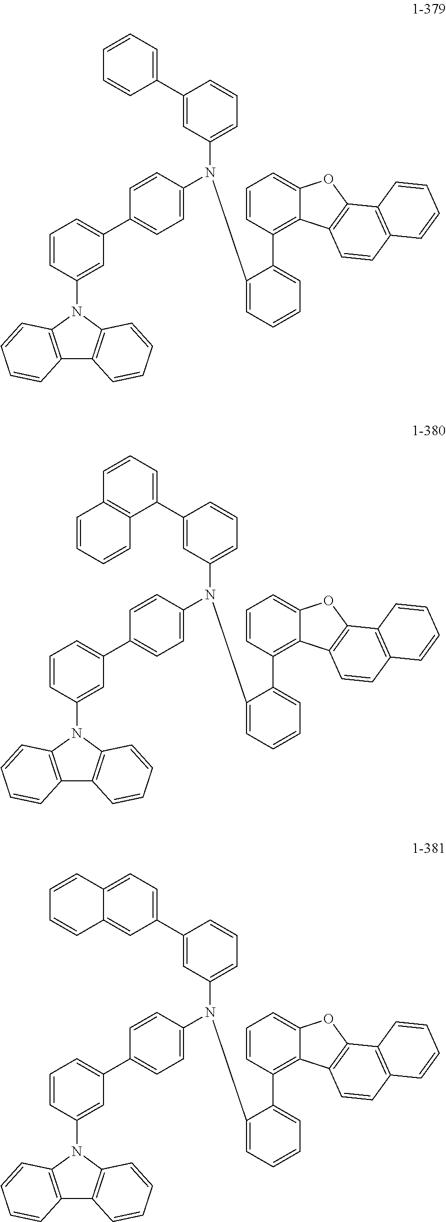 [SMILES: c1ccc(-c2cccc(N(c3ccc(-c4cccc(-n5c6ccccc6c6ccccc65)c4)cc3)c3ccccc3-c3cccc4oc5c6ccccc6ccc5c34)c2)cc1 ; c1cc(-c2cccc3ccccc23)cc(N(c2ccc(-c3cccc(-n4c5ccccc5c5ccccc54)c3)cc2)c2ccccc2-c2cccc3oc4c5ccccc5ccc4c23)c1 ; c1cc(-c2ccc3ccccc3c2)cc(N(c2ccc(-c3cccc(-n4c5ccccc5c5ccccc54)c3)cc2)c2ccccc2-c2cccc3oc4c5ccccc5ccc4c23)c1]

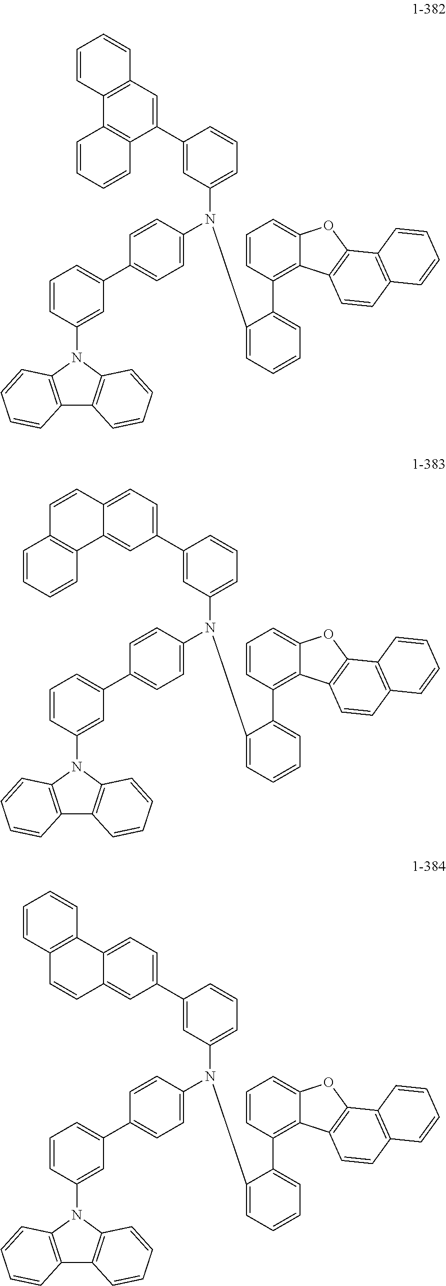 [SMILES: c1cc(-c2cc3ccccc3c3ccccc23)cc(N(c2ccc(-c3cccc(-n4c5ccccc5c5ccccc54)c3)cc2)c2ccccc2-c2cccc3oc4c5ccccc5ccc4c23)c1 ; c1cc(-c2ccc3ccc4ccccc4c3c2)cc(N(c2ccc(-c3cccc(-n4c5ccccc5c5ccccc54)c3)cc2)c2ccccc2-c2cccc3oc4c5ccccc5ccc4c23)c1 ; c1cc(-c2ccc3c(ccc4ccccc43)c2)cc(N(c2ccc(-c3cccc(-n4c5ccccc5c5ccccc54)c3)cc2)c2ccccc2-c2cccc3oc4c5ccccc5ccc4c23)c1]

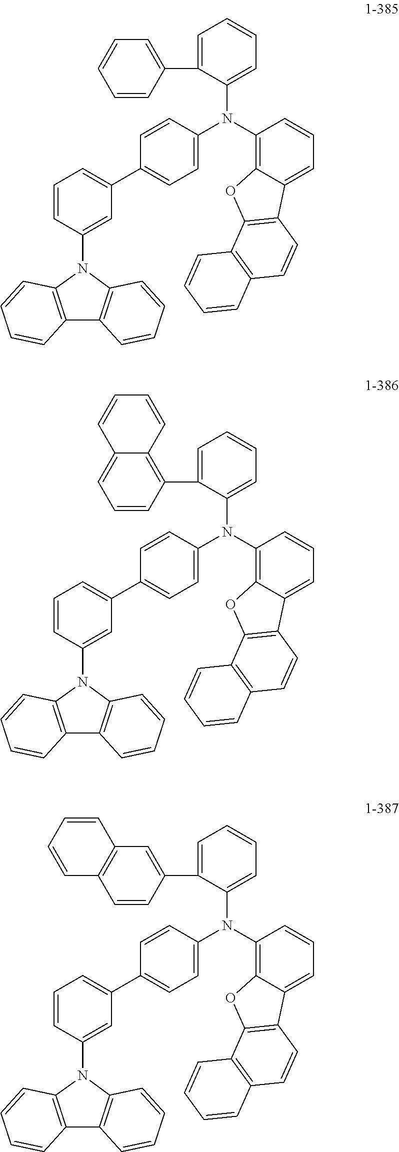 [SMILES: c1ccc(-c2ccccc2N(c2ccc(-c3cccc(-n4c5ccccc5c5ccccc54)c3)cc2)c2cccc3c2oc2c4ccccc4ccc32)cc1 ; c1cc(-c2ccc(N(c3ccccc3-c3cccc4ccccc34)c3cccc4c3oc3c5ccccc5ccc43)cc2)cc(-n2c3ccccc3c3ccccc32)c1 ; c1cc(-c2ccc(N(c3ccccc3-c3ccc4ccccc4c3)c3cccc4c3oc3c5ccccc5ccc43)cc2)cc(-n2c3ccccc3c3ccccc32)c1]

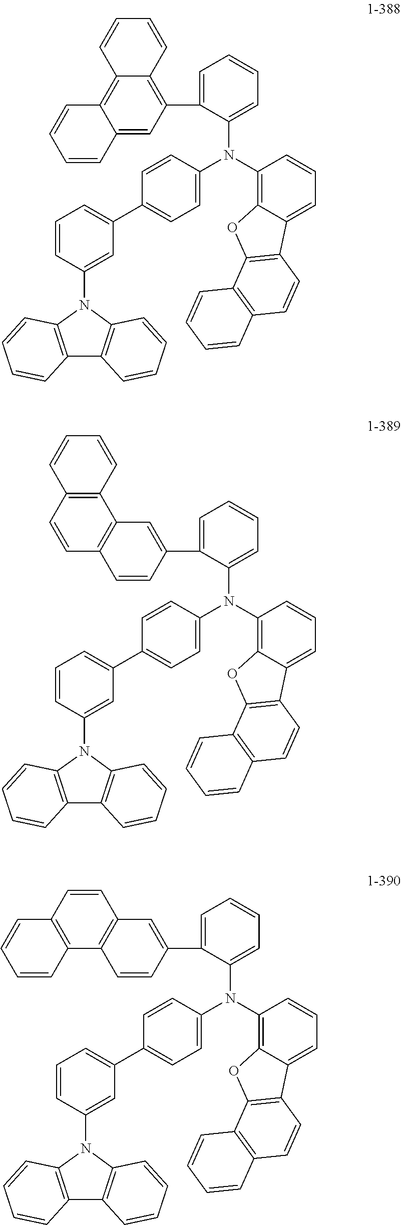 [SMILES: c1cc(-c2ccc(N(c3ccccc3-c3cc4ccccc4c4ccccc34)c3cccc4c3oc3c5ccccc5ccc43)cc2)cc(-n2c3ccccc3c3ccccc32)c1 ; c1cc(-c2ccc(N(c3ccccc3-c3ccc4ccc5ccccc5c4c3)c3cccc4c3oc3c5ccccc5ccc43)cc2)cc(-n2c3ccccc3c3ccccc32)c1 ; c1cc(-c2ccc(N(c3ccccc3-c3ccc4c(ccc5ccccc54)c3)c3cccc4c3oc3c5ccccc5ccc43)cc2)cc(-n2c3ccccc3c3ccccc32)c1]

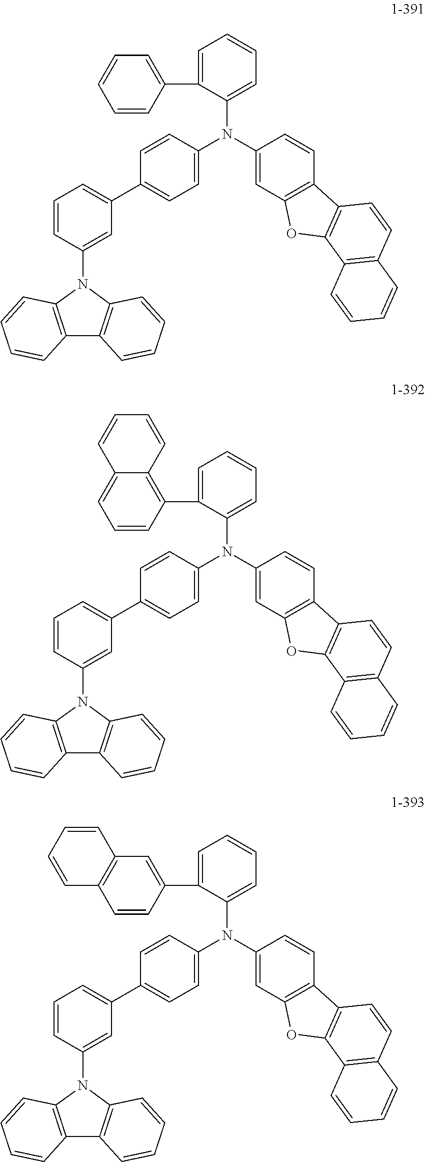 [SMILES: c1ccc(-c2ccccc2N(c2ccc(-c3cccc(-n4c5ccccc5c5ccccc54)c3)cc2)c2ccc3c(c2)oc2c4ccccc4ccc32)cc1 ; c1cc(-c2ccc(N(c3ccc4c(c3)oc3c5ccccc5ccc43)c3ccccc3-c3cccc4ccccc34)cc2)cc(-n2c3ccccc3c3ccccc32)c1 ; c1cc(-c2ccc(N(c3ccc4c(c3)oc3c5ccccc5ccc43)c3ccccc3-c3ccc4ccccc4c3)cc2)cc(-n2c3ccccc3c3ccccc32)c1]

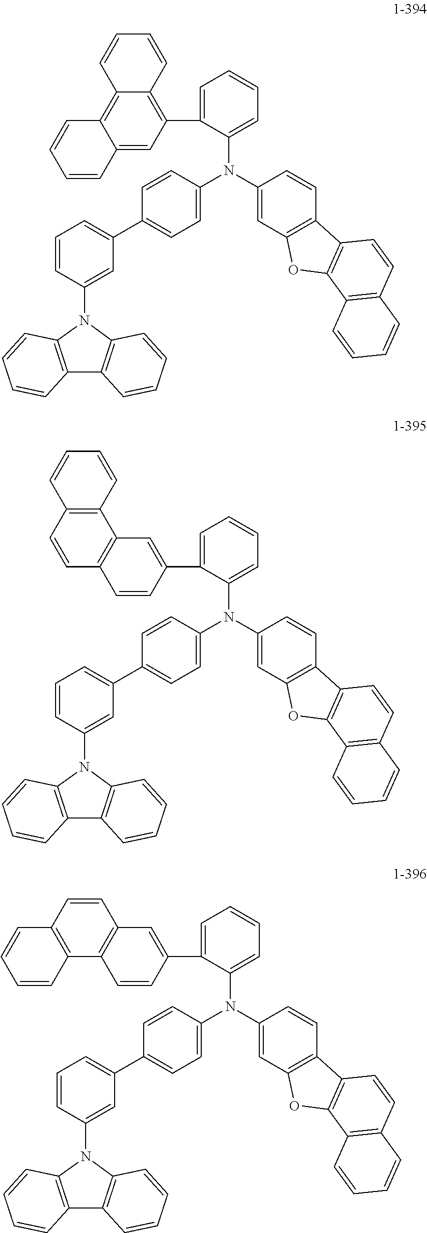 [SMILES: c1cc(-c2ccc(N(c3ccc4c(c3)oc3c5ccccc5ccc43)c3ccccc3-c3cc4ccccc4c4ccccc34)cc2)cc(-n2c3ccccc3c3ccccc32)c1 ; c1cc(-c2ccc(N(c3ccc4c(c3)oc3c5ccccc5ccc43)c3ccccc3-c3ccc4ccc5ccccc5c4c3)cc2)cc(-n2c3ccccc3c3ccccc32)c1 ; c1cc(-c2ccc(N(c3ccc4c(c3)oc3c5ccccc5ccc43)c3ccccc3-c3ccc4c(ccc5ccccc54)c3)cc2)cc(-n2c3ccccc3c3ccccc32)c1]

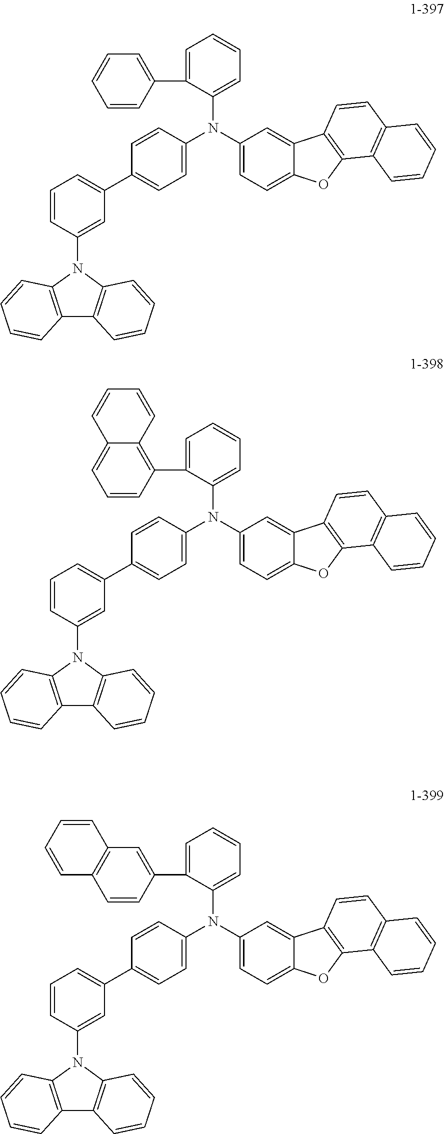 [SMILES: c1ccc(-c2ccccc2N(c2ccc(-c3cccc(-n4c5ccccc5c5ccccc54)c3)cc2)c2ccc3oc4c5ccccc5ccc4c3c2)cc1 ; c1cc(-c2ccc(N(c3ccc4oc5c6ccccc6ccc5c4c3)c3ccccc3-c3cccc4ccccc34)cc2)cc(-n2c3ccccc3c3ccccc32)c1 ; c1cc(-c2ccc(N(c3ccc4oc5c6ccccc6ccc5c4c3)c3ccccc3-c3ccc4ccccc4c3)cc2)cc(-n2c3ccccc3c3ccccc32)c1]

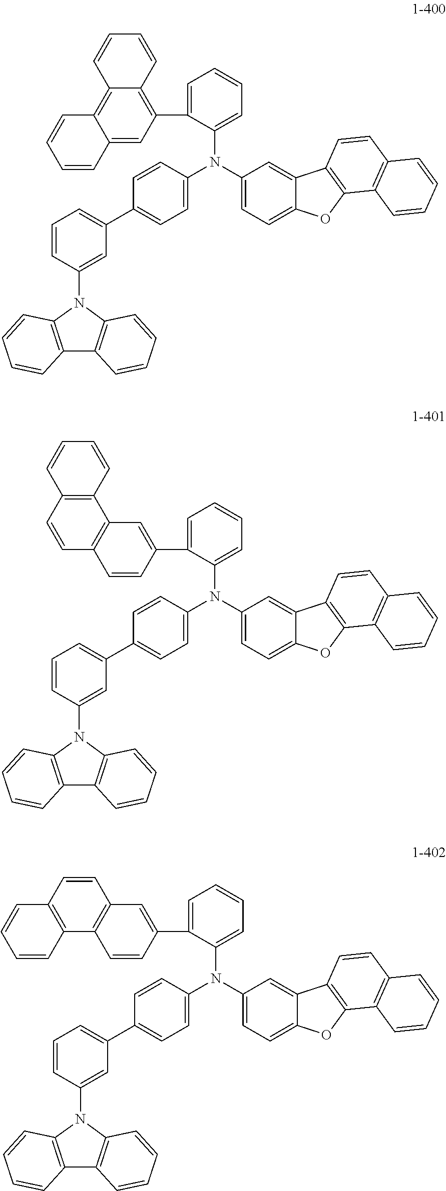 [SMILES: c1cc(-c2ccc(N(c3ccc4oc5c6ccccc6ccc5c4c3)c3ccccc3-c3cc4ccccc4c4ccccc34)cc2)cc(-n2c3ccccc3c3ccccc32)c1 ; c1cc(-c2ccc(N(c3ccc4oc5c6ccccc6ccc5c4c3)c3ccccc3-c3ccc4ccc5ccccc5c4c3)cc2)cc(-n2c3ccccc3c3ccccc32)c1 ; c1cc(-c2ccc(N(c3ccc4oc5c6ccccc6ccc5c4c3)c3ccccc3-c3ccc4c(ccc5ccccc54)c3)cc2)cc(-n2c3ccccc3c3ccccc32)c1]

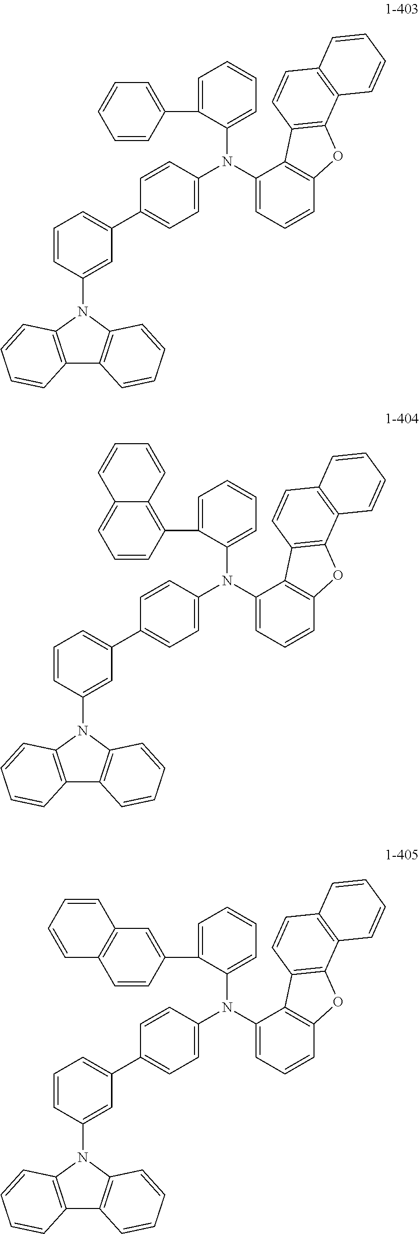 [SMILES: c1ccc(-c2ccccc2N(c2ccc(-c3cccc(-n4c5ccccc5c5ccccc54)c3)cc2)c2cccc3oc4c5ccccc5ccc4c23)cc1 ; c1cc(-c2ccc(N(c3ccccc3-c3cccc4ccccc34)c3cccc4oc5c6ccccc6ccc5c34)cc2)cc(-n2c3ccccc3c3ccccc32)c1 ; c1cc(-c2ccc(N(c3ccccc3-c3ccc4ccccc4c3)c3cccc4oc5c6ccccc6ccc5c34)cc2)cc(-n2c3ccccc3c3ccccc32)c1]

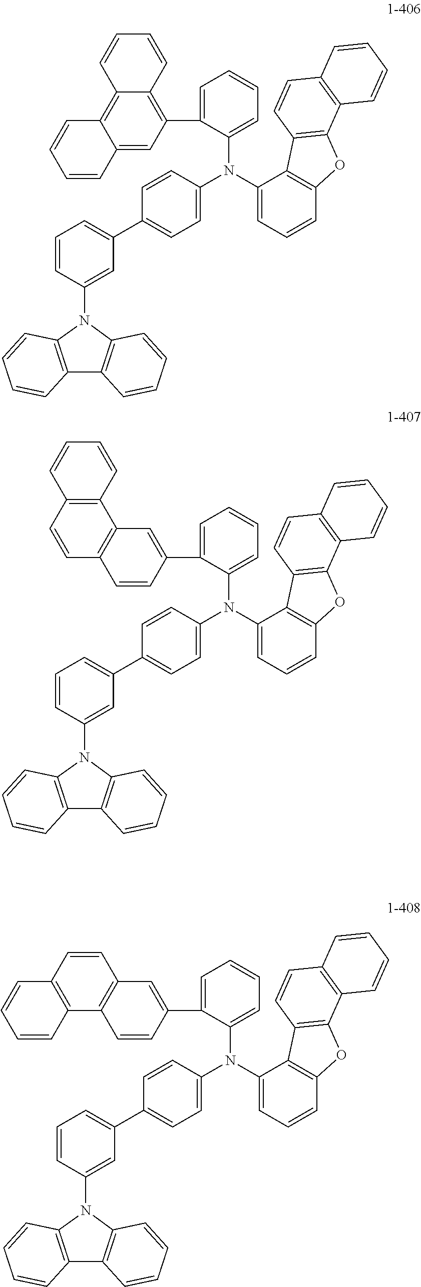 [SMILES: c1cc(-c2ccc(N(c3ccccc3-c3cc4ccccc4c4ccccc34)c3cccc4oc5c6ccccc6ccc5c34)cc2)cc(-n2c3ccccc3c3ccccc32)c1 ; c1cc(-c2ccc(N(c3ccccc3-c3ccc4ccc5ccccc5c4c3)c3cccc4oc5c6ccccc6ccc5c34)cc2)cc(-n2c3ccccc3c3ccccc32)c1 ; c1cc(-c2ccc(N(c3ccccc3-c3ccc4c(ccc5ccccc54)c3)c3cccc4oc5c6ccccc6ccc5c34)cc2)cc(-n2c3ccccc3c3ccccc32)c1]

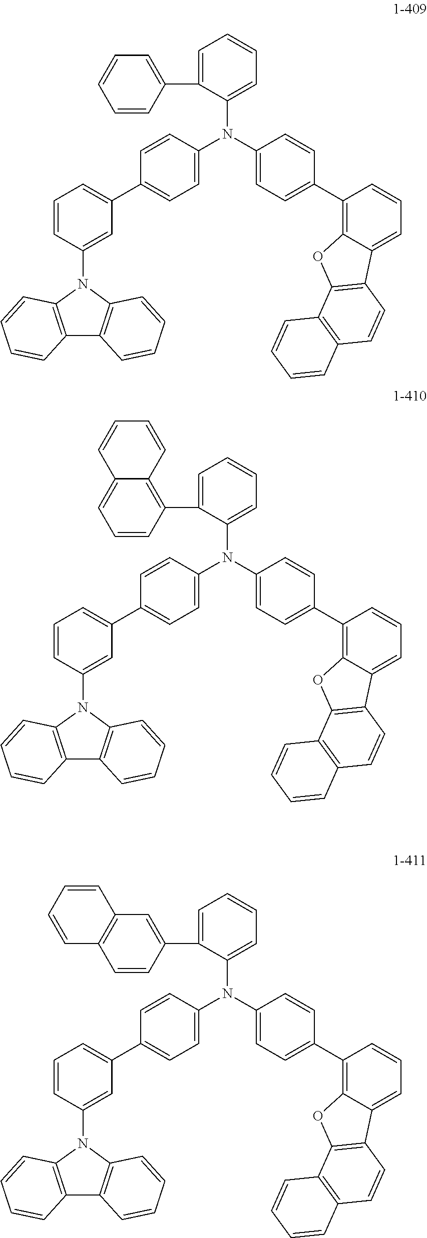 [SMILES: c1ccc(-c2ccccc2N(c2ccc(-c3cccc(-n4c5ccccc5c5ccccc54)c3)cc2)c2ccc(-c3cccc4c3oc3c5ccccc5ccc43)cc2)cc1 ; c1cc(-c2ccc(N(c3ccc(-c4cccc5c4oc4c6ccccc6ccc54)cc3)c3ccccc3-c3cccc4ccccc34)cc2)cc(-n2c3ccccc3c3ccccc32)c1 ; c1cc(-c2ccc(N(c3ccc(-c4cccc5c4oc4c6ccccc6ccc54)cc3)c3ccccc3-c3ccc4ccccc4c3)cc2)cc(-n2c3ccccc3c3ccccc32)c1]

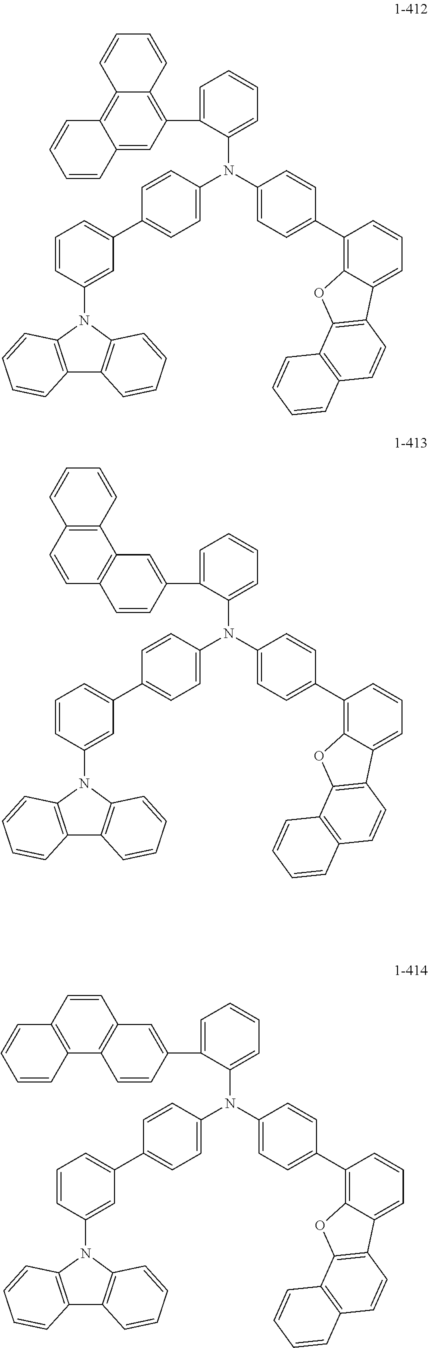 [SMILES: c1cc(-c2ccc(N(c3ccc(-c4cccc5c4oc4c6ccccc6ccc54)cc3)c3ccccc3-c3cc4ccccc4c4ccccc34)cc2)cc(-n2c3ccccc3c3ccccc32)c1 ; c1cc(-c2ccc(N(c3ccc(-c4cccc5c4oc4c6ccccc6ccc54)cc3)c3ccccc3-c3ccc4ccc5ccccc5c4c3)cc2)cc(-n2c3ccccc3c3ccccc32)c1 ; c1cc(-c2ccc(N(c3ccc(-c4cccc5c4oc4c6ccccc6ccc54)cc3)c3ccccc3-c3ccc4c(ccc5ccccc54)c3)cc2)cc(-n2c3ccccc3c3ccccc32)c1]

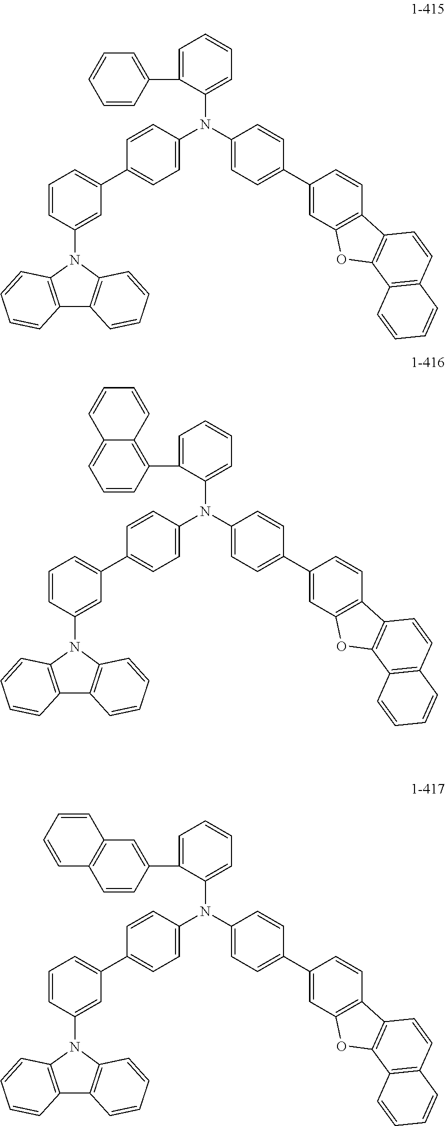 [SMILES: c1ccc(-c2ccccc2N(c2ccc(-c3cccc(-n4c5ccccc5c5ccccc54)c3)cc2)c2ccc(-c3ccc4c(c3)oc3c5ccccc5ccc43)cc2)cc1 ; c1cc(-c2ccc(N(c3ccc(-c4ccc5c(c4)oc4c6ccccc6ccc54)cc3)c3ccccc3-c3cccc4ccccc34)cc2)cc(-n2c3ccccc3c3ccccc32)c1 ; c1cc(-c2ccc(N(c3ccc(-c4ccc5c(c4)oc4c6ccccc6ccc54)cc3)c3ccccc3-c3ccc4ccccc4c3)cc2)cc(-n2c3ccccc3c3ccccc32)c1]

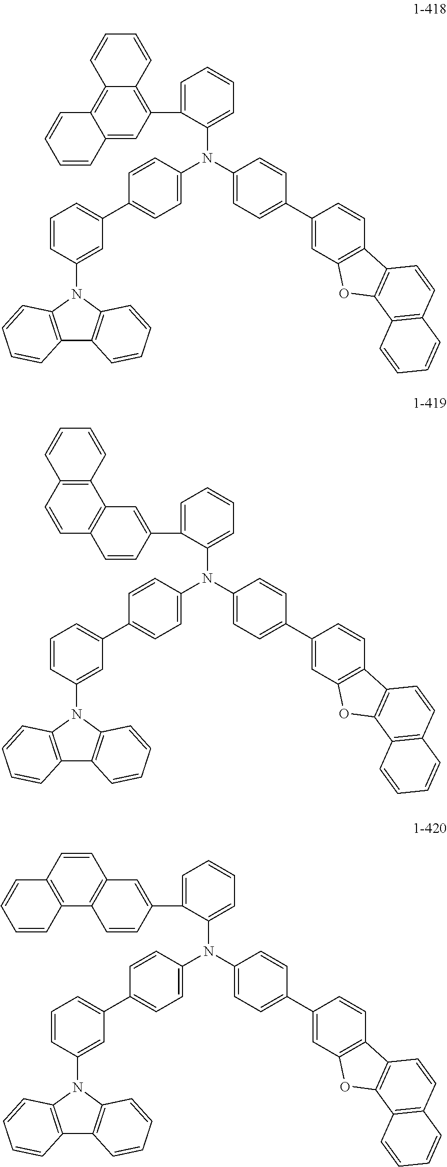 [SMILES: c1cc(-c2ccc(N(c3ccc(-c4ccc5c(c4)oc4c6ccccc6ccc54)cc3)c3ccccc3-c3cc4ccccc4c4ccccc34)cc2)cc(-n2c3ccccc3c3ccccc32)c1 ; c1cc(-c2ccc(N(c3ccc(-c4ccc5c(c4)oc4c6ccccc6ccc54)cc3)c3ccccc3-c3ccc4ccc5ccccc5c4c3)cc2)cc(-n2c3ccccc3c3ccccc32)c1 ; c1cc(-c2ccc(N(c3ccc(-c4ccc5c(c4)oc4c6ccccc6ccc54)cc3)c3ccccc3-c3ccc4c(ccc5ccccc54)c3)cc2)cc(-n2c3ccccc3c3ccccc32)c1]

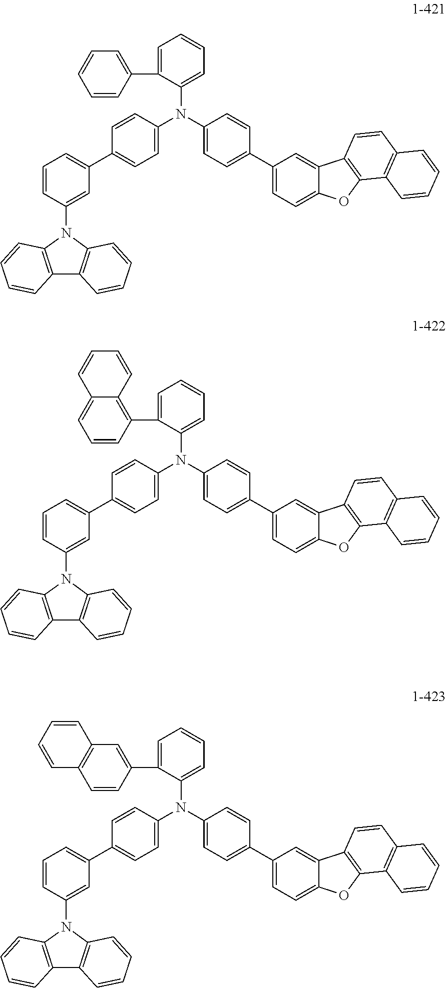 [SMILES: c1ccc(-c2ccccc2N(c2ccc(-c3cccc(-n4c5ccccc5c5ccccc54)c3)cc2)c2ccc(-c3ccc4oc5c6ccccc6ccc5c4c3)cc2)cc1 ; c1cc(-c2ccc(N(c3ccc(-c4ccc5oc6c7ccccc7ccc6c5c4)cc3)c3ccccc3-c3cccc4ccccc34)cc2)cc(-n2c3ccccc3c3ccccc32)c1 ; c1cc(-c2ccc(N(c3ccc(-c4ccc5oc6c7ccccc7ccc6c5c4)cc3)c3ccccc3-c3ccc4ccccc4c3)cc2)cc(-n2c3ccccc3c3ccccc32)c1]

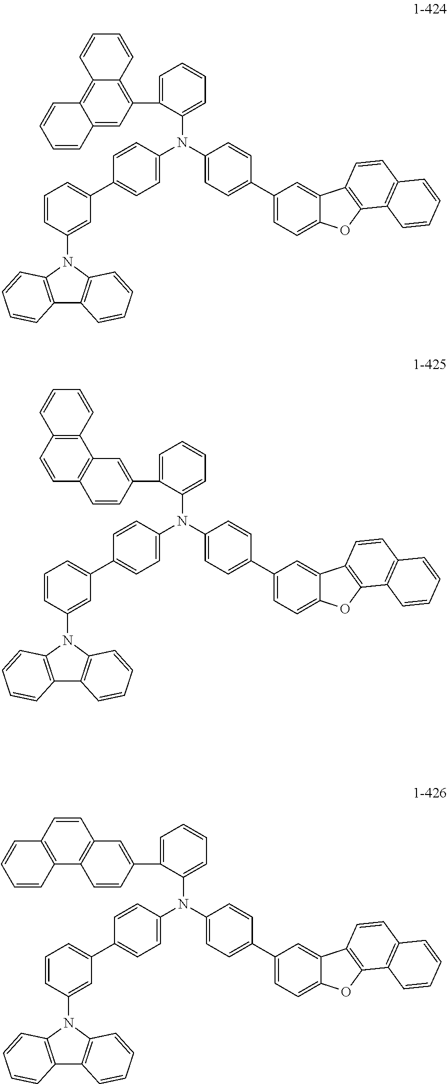 [SMILES: c1cc(-c2ccc(N(c3ccc(-c4ccc5oc6c7ccccc7ccc6c5c4)cc3)c3ccccc3-c3cc4ccccc4c4ccccc34)cc2)cc(-n2c3ccccc3c3ccccc32)c1 ; c1cc(-c2ccc(N(c3ccc(-c4ccc5oc6c7ccccc7ccc6c5c4)cc3)c3ccccc3-c3ccc4ccc5ccccc5c4c3)cc2)cc(-n2c3ccccc3c3ccccc32)c1 ; c1cc(-c2ccc(N(c3ccc(-c4ccc5oc6c7ccccc7ccc6c5c4)cc3)c3ccccc3-c3ccc4c(ccc5ccccc54)c3)cc2)cc(-n2c3ccccc3c3ccccc32)c1]

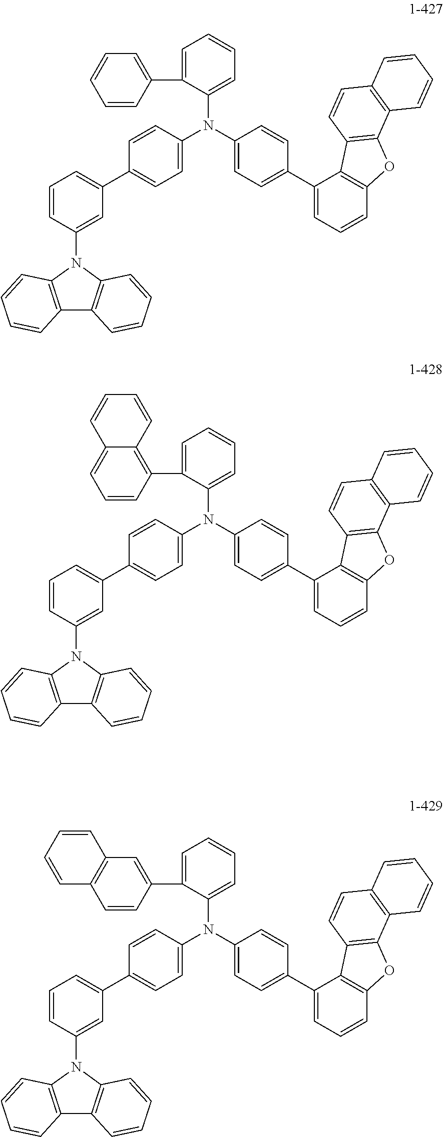 [SMILES: c1ccc(-c2ccccc2N(c2ccc(-c3cccc(-n4c5ccccc5c5ccccc54)c3)cc2)c2ccc(-c3cccc4oc5c6ccccc6ccc5c34)cc2)cc1 ; c1cc(-c2ccc(N(c3ccc(-c4cccc5oc6c7ccccc7ccc6c45)cc3)c3ccccc3-c3cccc4ccccc34)cc2)cc(-n2c3ccccc3c3ccccc32)c1 ; c1cc(-c2ccc(N(c3ccc(-c4cccc5oc6c7ccccc7ccc6c45)cc3)c3ccccc3-c3ccc4ccccc4c3)cc2)cc(-n2c3ccccc3c3ccccc32)c1]

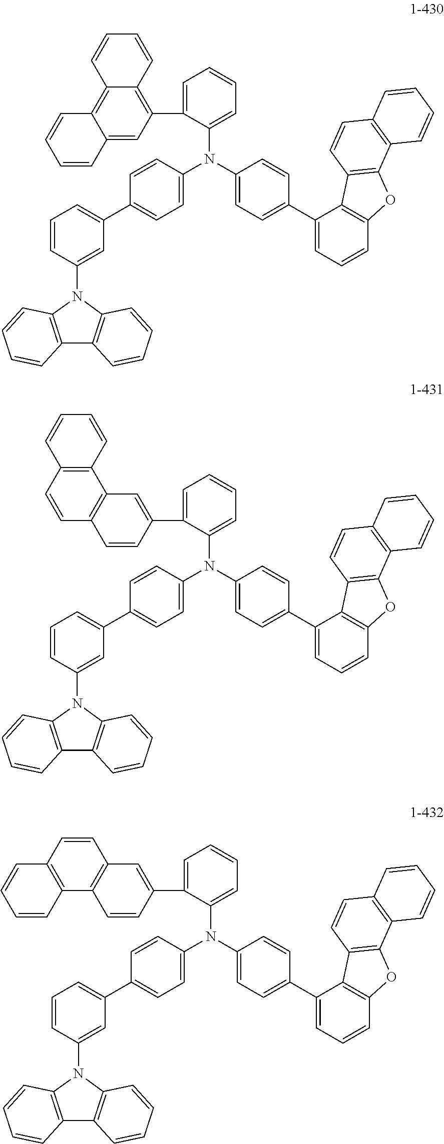 [SMILES: c1cc(-c2ccc(N(c3ccc(-c4cccc5oc6c7ccccc7ccc6c45)cc3)c3ccccc3-c3cc4ccccc4c4ccccc34)cc2)cc(-n2c3ccccc3c3ccccc32)c1 ; c1cc(-c2ccc(N(c3ccc(-c4cccc5oc6c7ccccc7ccc6c45)cc3)c3ccccc3-c3ccc4ccc5ccccc5c4c3)cc2)cc(-n2c3ccccc3c3ccccc32)c1 ; c1cc(-c2ccc(N(c3ccc(-c4cccc5oc6c7ccccc7ccc6c45)cc3)c3ccccc3-c3ccc4c(ccc5ccccc54)c3)cc2)cc(-n2c3ccccc3c3ccccc32)c1]

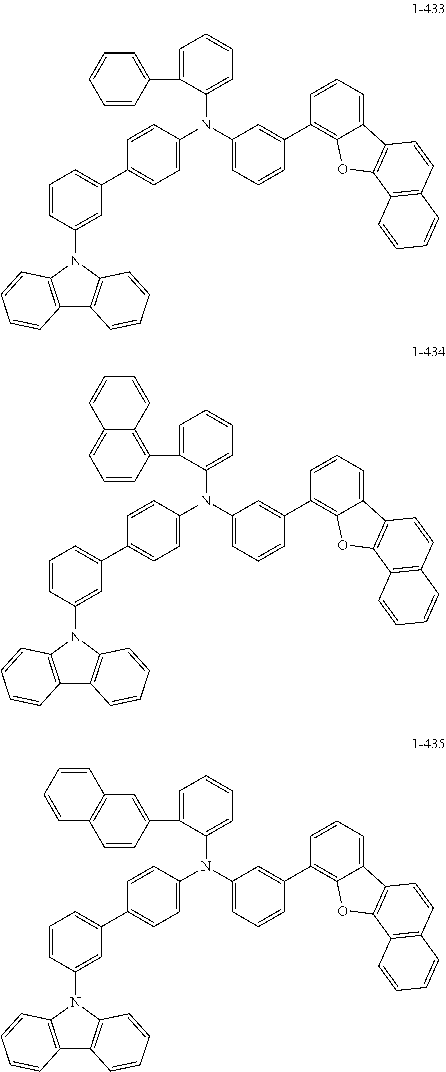 [SMILES: c1ccc(-c2ccccc2N(c2ccc(-c3cccc(-n4c5ccccc5c5ccccc54)c3)cc2)c2cccc(-c3cccc4c3oc3c5ccccc5ccc43)c2)cc1 ; c1cc(-c2cccc3c2oc2c4ccccc4ccc32)cc(N(c2ccc(-c3cccc(-n4c5ccccc5c5ccccc54)c3)cc2)c2ccccc2-c2cccc3ccccc23)c1 ; c1cc(-c2cccc3c2oc2c4ccccc4ccc32)cc(N(c2ccc(-c3cccc(-n4c5ccccc5c5ccccc54)c3)cc2)c2ccccc2-c2ccc3ccccc3c2)c1]

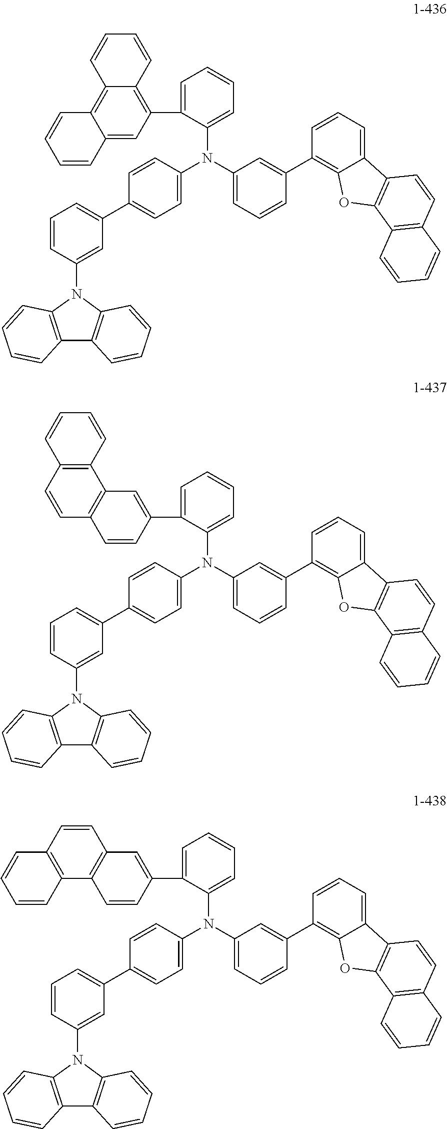 [SMILES: c1cc(-c2cccc3c2oc2c4ccccc4ccc32)cc(N(c2ccc(-c3cccc(-n4c5ccccc5c5ccccc54)c3)cc2)c2ccccc2-c2cc3ccccc3c3ccccc23)c1 ; c1cc(-c2cccc3c2oc2c4ccccc4ccc32)cc(N(c2ccc(-c3cccc(-n4c5ccccc5c5ccccc54)c3)cc2)c2ccccc2-c2ccc3ccc4ccccc4c3c2)c1 ; c1cc(-c2cccc3c2oc2c4ccccc4ccc32)cc(N(c2ccc(-c3cccc(-n4c5ccccc5c5ccccc54)c3)cc2)c2ccccc2-c2ccc3c(ccc4ccccc43)c2)c1]

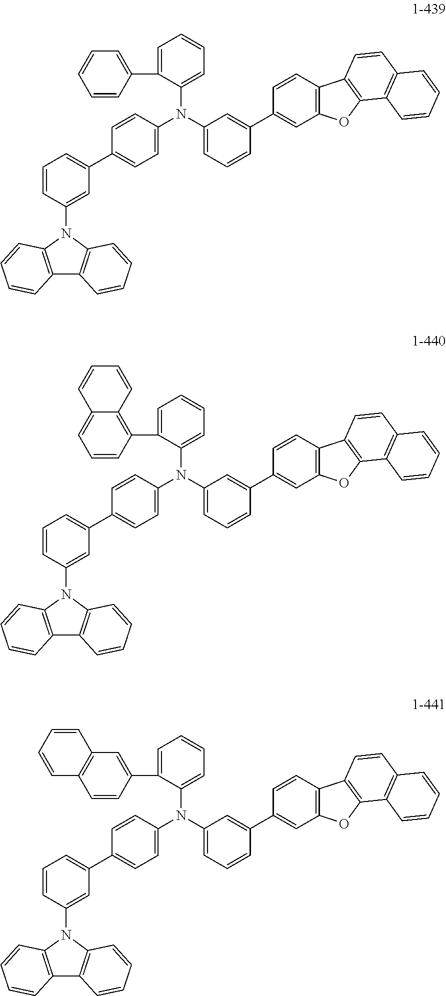 [SMILES: c1ccc(-c2ccccc2N(c2ccc(-c3cccc(-n4c5ccccc5c5ccccc54)c3)cc2)c2cccc(-c3ccc4c(c3)oc3c5ccccc5ccc43)c2)cc1 ; c1cc(-c2ccc3c(c2)oc2c4ccccc4ccc32)cc(N(c2ccc(-c3cccc(-n4c5ccccc5c5ccccc54)c3)cc2)c2ccccc2-c2cccc3ccccc23)c1 ; c1cc(-c2ccc3c(c2)oc2c4ccccc4ccc32)cc(N(c2ccc(-c3cccc(-n4c5ccccc5c5ccccc54)c3)cc2)c2ccccc2-c2ccc3ccccc3c2)c1]

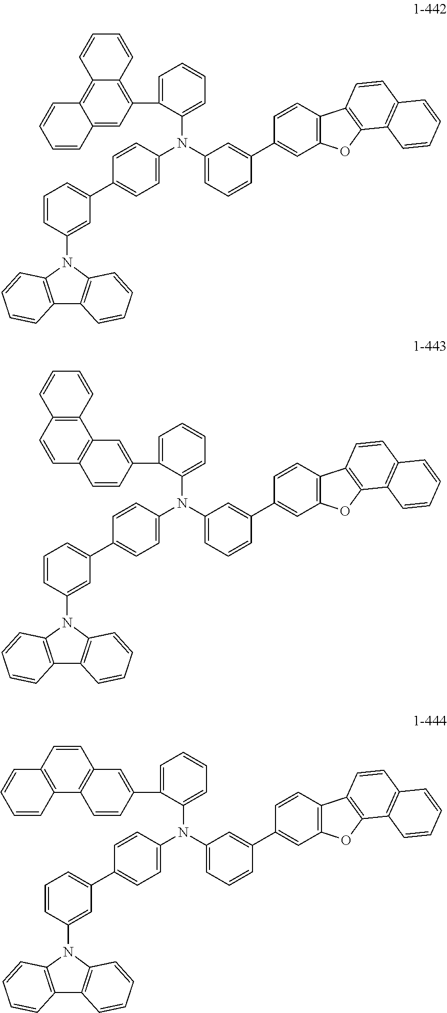 [SMILES: c1cc(-c2ccc3c(c2)oc2c4ccccc4ccc32)cc(N(c2ccc(-c3cccc(-n4c5ccccc5c5ccccc54)c3)cc2)c2ccccc2-c2cc3ccccc3c3ccccc23)c1 ; c1cc(-c2ccc3c(c2)oc2c4ccccc4ccc32)cc(N(c2ccc(-c3cccc(-n4c5ccccc5c5ccccc54)c3)cc2)c2ccccc2-c2ccc3ccc4ccccc4c3c2)c1 ; c1cc(-c2ccc3c(c2)oc2c4ccccc4ccc32)cc(N(c2ccc(-c3cccc(-n4c5ccccc5c5ccccc54)c3)cc2)c2ccccc2-c2ccc3c(ccc4ccccc43)c2)c1]

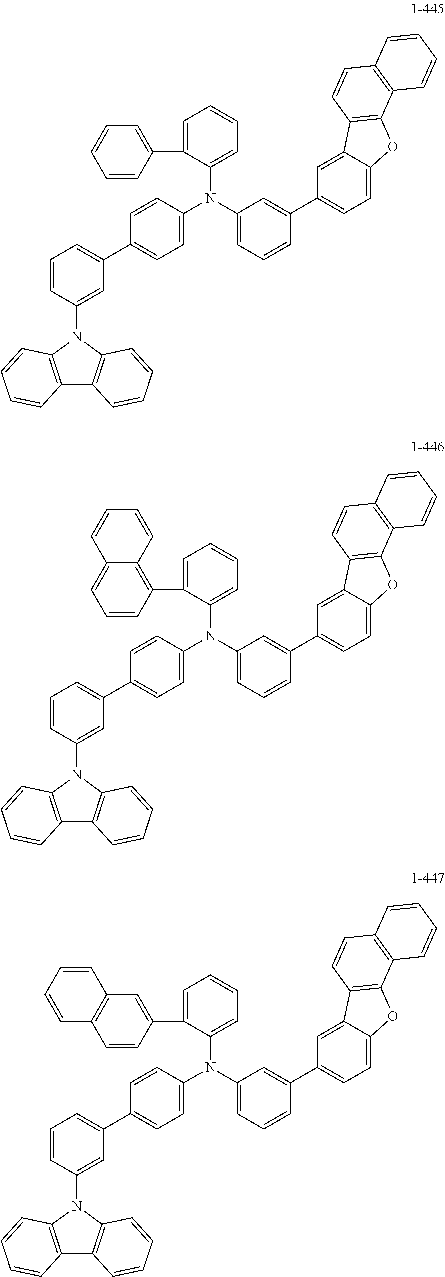 [SMILES: c1ccc(-c2ccccc2N(c2ccc(-c3cccc(-n4c5ccccc5c5ccccc54)c3)cc2)c2cccc(-c3ccc4oc5c6ccccc6ccc5c4c3)c2)cc1 ; c1cc(-c2ccc3oc4c5ccccc5ccc4c3c2)cc(N(c2ccc(-c3cccc(-n4c5ccccc5c5ccccc54)c3)cc2)c2ccccc2-c2cccc3ccccc23)c1 ; c1cc(-c2ccc3oc4c5ccccc5ccc4c3c2)cc(N(c2ccc(-c3cccc(-n4c5ccccc5c5ccccc54)c3)cc2)c2ccccc2-c2ccc3ccccc3c2)c1]

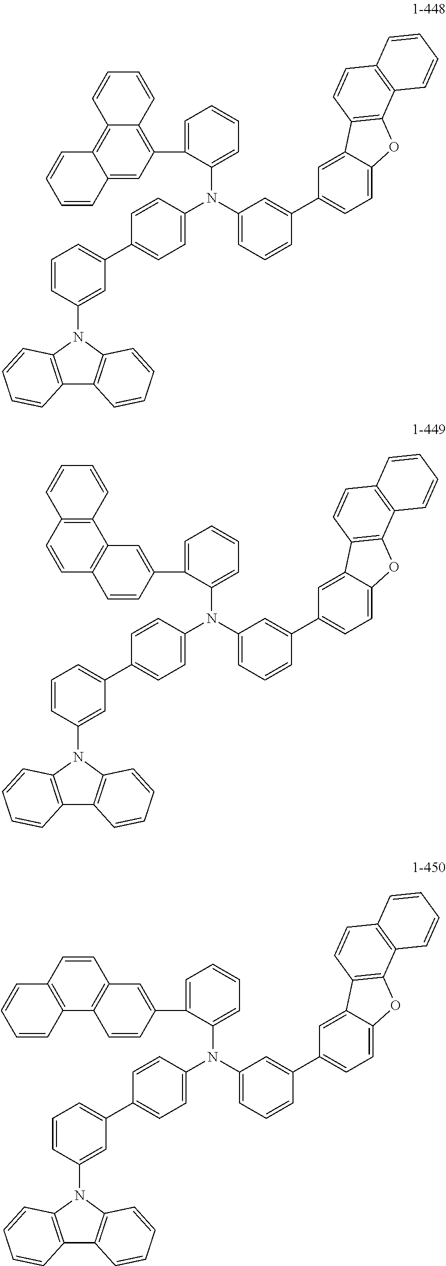 [SMILES: c1cc(-c2ccc3oc4c5ccccc5ccc4c3c2)cc(N(c2ccc(-c3cccc(-n4c5ccccc5c5ccccc54)c3)cc2)c2ccccc2-c2cc3ccccc3c3ccccc23)c1 ; c1cc(-c2ccc3oc4c5ccccc5ccc4c3c2)cc(N(c2ccc(-c3cccc(-n4c5ccccc5c5ccccc54)c3)cc2)c2ccccc2-c2ccc3ccc4ccccc4c3c2)c1 ; c1cc(-c2ccc3oc4c5ccccc5ccc4c3c2)cc(N(c2ccc(-c3cccc(-n4c5ccccc5c5ccccc54)c3)cc2)c2ccccc2-c2ccc3c(ccc4ccccc43)c2)c1]

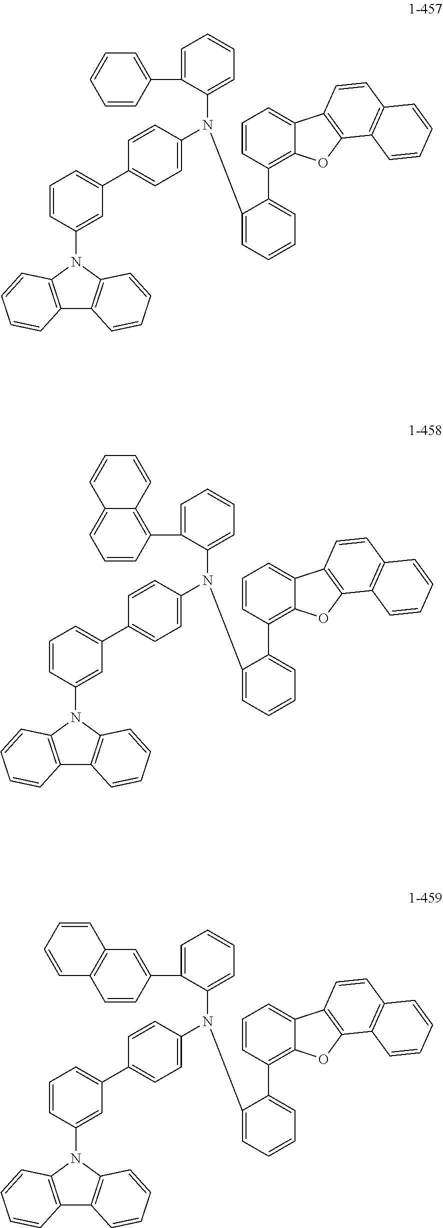 [SMILES: c1ccc(-c2ccccc2N(c2ccc(-c3cccc(-n4c5ccccc5c5ccccc54)c3)cc2)c2ccccc2-c2cccc3c2oc2c4ccccc4ccc32)cc1 ; c1cc(-c2ccc(N(c3ccccc3-c3cccc4ccccc34)c3ccccc3-c3cccc4c3oc3c5ccccc5ccc43)cc2)cc(-n2c3ccccc3c3ccccc32)c1 ; c1cc(-c2ccc(N(c3ccccc3-c3ccc4ccccc4c3)c3ccccc3-c3cccc4c3oc3c5ccccc5ccc43)cc2)cc(-n2c3ccccc3c3ccccc32)c1]

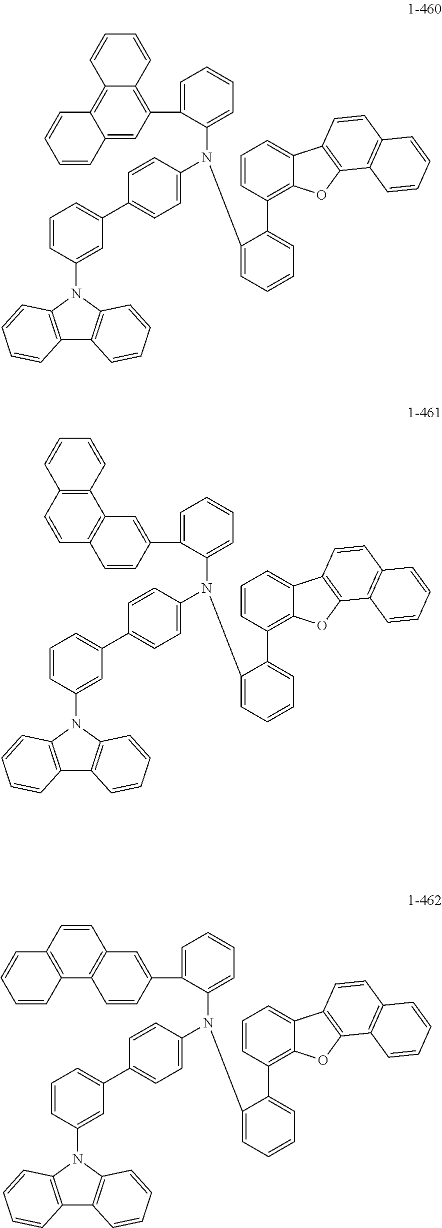 [SMILES: c1cc(-c2ccc(N(c3ccccc3-c3cc4ccccc4c4ccccc34)c3ccccc3-c3cccc4c3oc3c5ccccc5ccc43)cc2)cc(-n2c3ccccc3c3ccccc32)c1 ; c1cc(-c2ccc(N(c3ccccc3-c3ccc4ccc5ccccc5c4c3)c3ccccc3-c3cccc4c3oc3c5ccccc5ccc43)cc2)cc(-n2c3ccccc3c3ccccc32)c1 ; c1cc(-c2ccc(N(c3ccccc3-c3ccc4c(ccc5ccccc54)c3)c3ccccc3-c3cccc4c3oc3c5ccccc5ccc43)cc2)cc(-n2c3ccccc3c3ccccc32)c1]

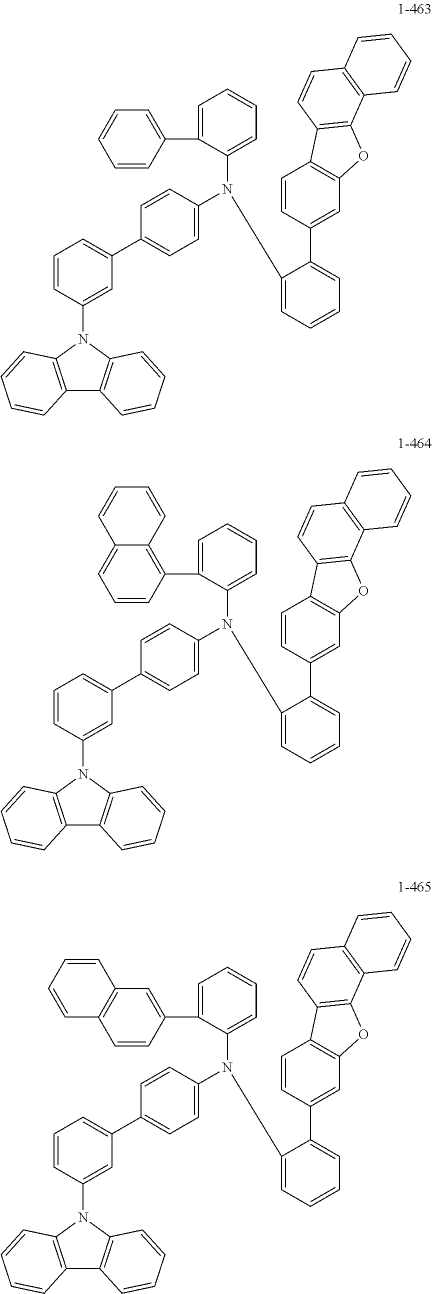 [SMILES: c1ccc(-c2ccccc2N(c2ccc(-c3cccc(-n4c5ccccc5c5ccccc54)c3)cc2)c2ccccc2-c2ccc3c(c2)oc2c4ccccc4ccc32)cc1 ; c1cc(-c2ccc(N(c3ccccc3-c3ccc4c(c3)oc3c5ccccc5ccc43)c3ccccc3-c3cccc4ccccc34)cc2)cc(-n2c3ccccc3c3ccccc32)c1 ; c1cc(-c2ccc(N(c3ccccc3-c3ccc4ccccc4c3)c3ccccc3-c3ccc4c(c3)oc3c5ccccc5ccc43)cc2)cc(-n2c3ccccc3c3ccccc32)c1]

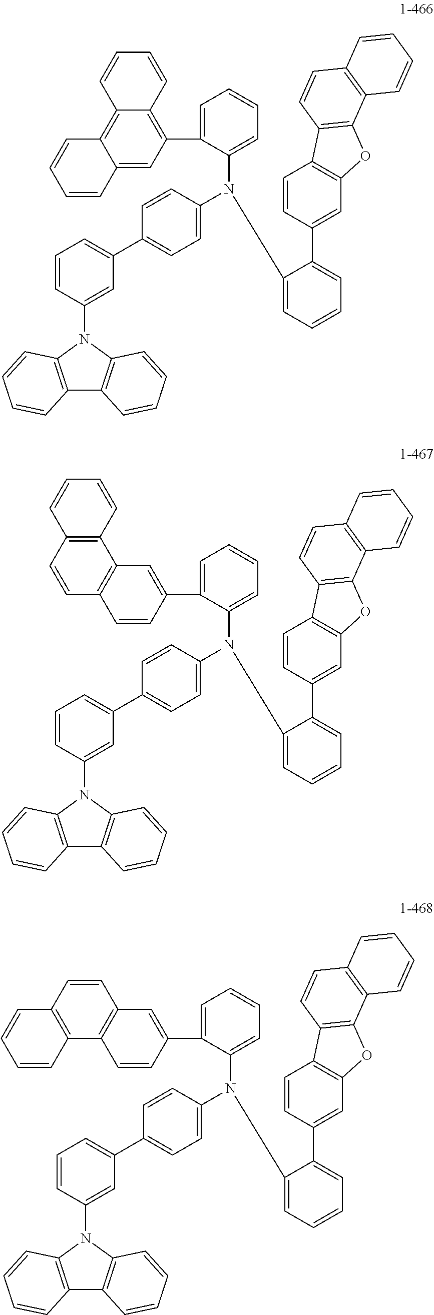 [SMILES: c1cc(-c2ccc(N(c3ccccc3-c3ccc4c(c3)oc3c5ccccc5ccc43)c3ccccc3-c3cc4ccccc4c4ccccc34)cc2)cc(-n2c3ccccc3c3ccccc32)c1 ; c1cc(-c2ccc(N(c3ccccc3-c3ccc4c(c3)oc3c5ccccc5ccc43)c3ccccc3-c3ccc4ccc5ccccc5c4c3)cc2)cc(-n2c3ccccc3c3ccccc32)c1 ; c1cc(-c2ccc(N(c3ccccc3-c3ccc4c(ccc5ccccc54)c3)c3ccccc3-c3ccc4c(c3)oc3c5ccccc5ccc43)cc2)cc(-n2c3ccccc3c3ccccc32)c1]

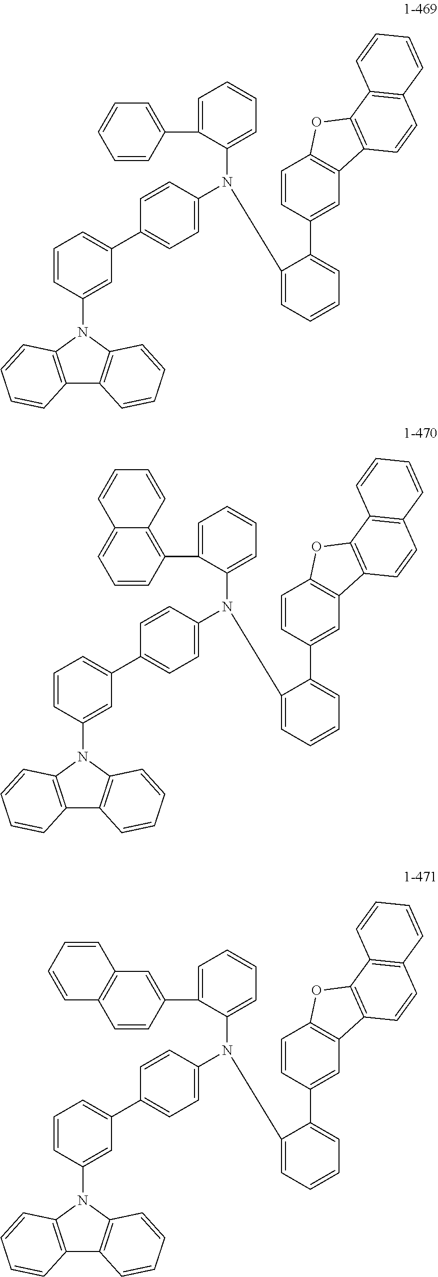 [SMILES: c1ccc(-c2ccccc2N(c2ccc(-c3cccc(-n4c5ccccc5c5ccccc54)c3)cc2)c2ccccc2-c2ccc3oc4c5ccccc5ccc4c3c2)cc1 ; c1cc(-c2ccc(N(c3ccccc3-c3ccc4oc5c6ccccc6ccc5c4c3)c3ccccc3-c3cccc4ccccc34)cc2)cc(-n2c3ccccc3c3ccccc32)c1 ; c1cc(-c2ccc(N(c3ccccc3-c3ccc4ccccc4c3)c3ccccc3-c3ccc4oc5c6ccccc6ccc5c4c3)cc2)cc(-n2c3ccccc3c3ccccc32)c1]

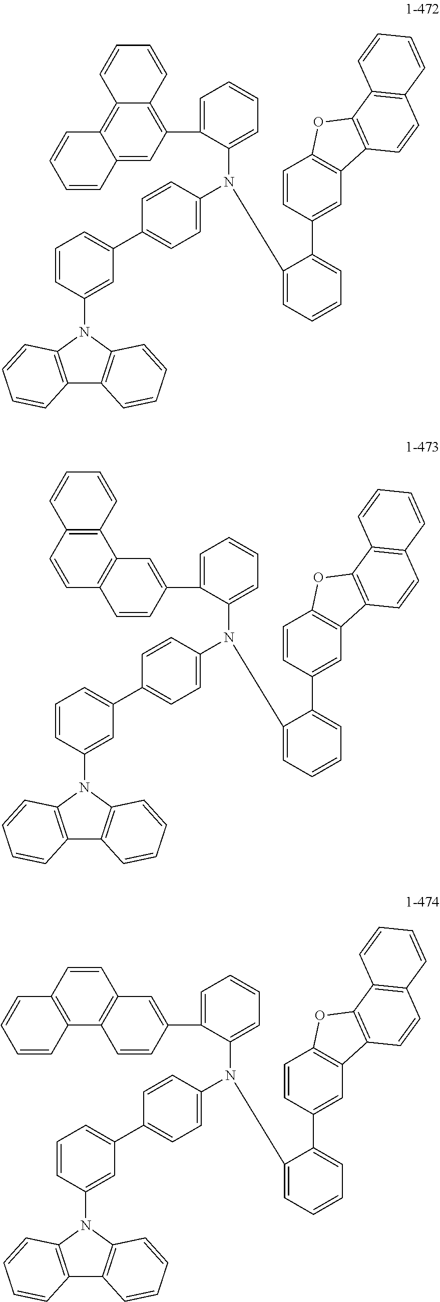 [SMILES: c1cc(-c2ccc(N(c3ccccc3-c3ccc4oc5c6ccccc6ccc5c4c3)c3ccccc3-c3cc4ccccc4c4ccccc34)cc2)cc(-n2c3ccccc3c3ccccc32)c1 ; c1cc(-c2ccc(N(c3ccccc3-c3ccc4ccc5ccccc5c4c3)c3ccccc3-c3ccc4oc5c6ccccc6ccc5c4c3)cc2)cc(-n2c3ccccc3c3ccccc32)c1 ; c1cc(-c2ccc(N(c3ccccc3-c3ccc4c(ccc5ccccc54)c3)c3ccccc3-c3ccc4oc5c6ccccc6ccc5c4c3)cc2)cc(-n2c3ccccc3c3ccccc32)c1]

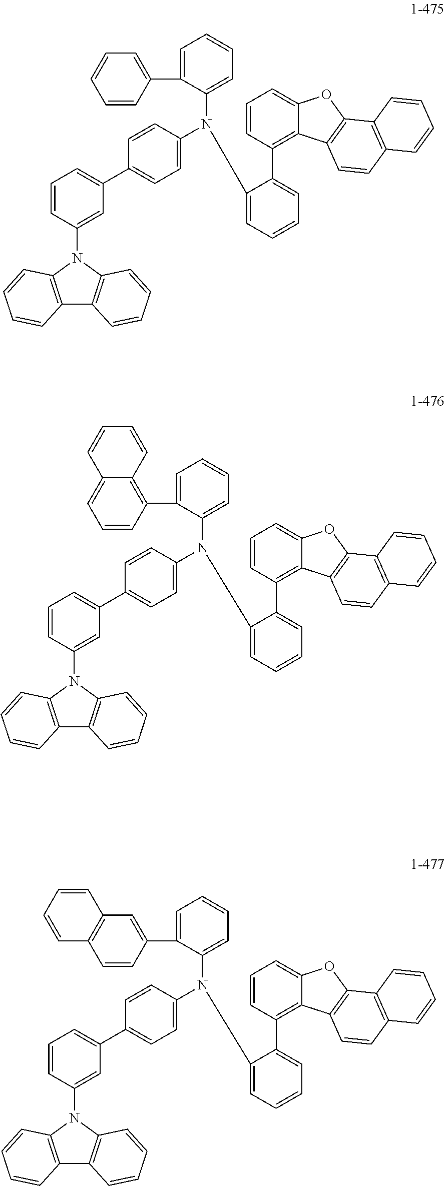 [SMILES: c1ccc(-c2ccccc2N(c2ccc(-c3cccc(-n4c5ccccc5c5ccccc54)c3)cc2)c2ccccc2-c2cccc3oc4c5ccccc5ccc4c23)cc1 ; c1cc(-c2ccc(N(c3ccccc3-c3cccc4ccccc34)c3ccccc3-c3cccc4oc5c6ccccc6ccc5c34)cc2)cc(-n2c3ccccc3c3ccccc32)c1 ; c1cc(-c2ccc(N(c3ccccc3-c3ccc4ccccc4c3)c3ccccc3-c3cccc4oc5c6ccccc6ccc5c34)cc2)cc(-n2c3ccccc3c3ccccc32)c1]

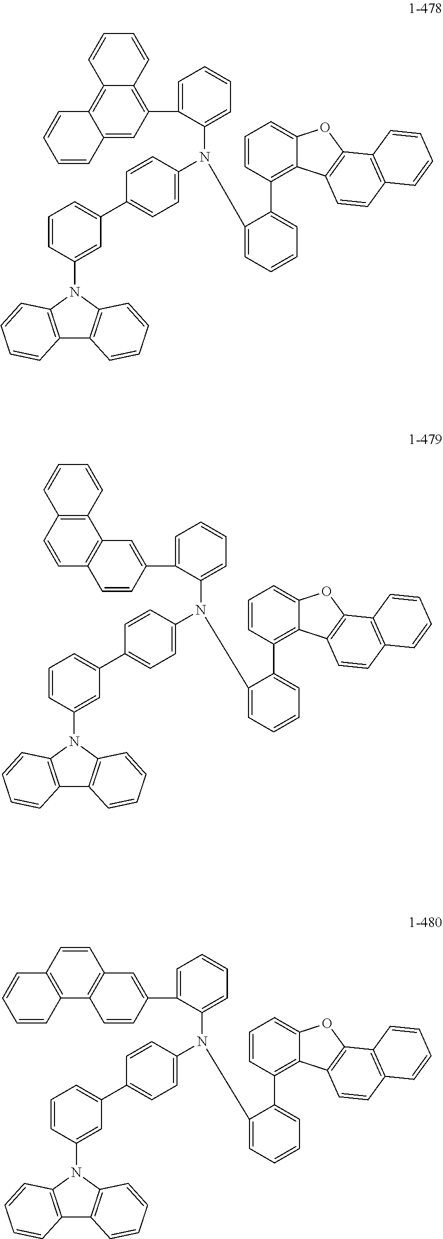 [SMILES: c1cc(-c2ccc(N(c3ccccc3-c3cc4ccccc4c4ccccc34)c3ccccc3-c3cccc4oc5c6ccccc6ccc5c34)cc2)cc(-n2c3ccccc3c3ccccc32)c1 ; c1cc(-c2ccc(N(c3ccccc3-c3ccc4ccc5ccccc5c4c3)c3ccccc3-c3cccc4oc5c6ccccc6ccc5c34)cc2)cc(-n2c3ccccc3c3ccccc32)c1 ; c1cc(-c2ccc(N(c3ccccc3-c3ccc4c(ccc5ccccc54)c3)c3ccccc3-c3cccc4oc5c6ccccc6ccc5c34)cc2)cc(-n2c3ccccc3c3ccccc32)c1]

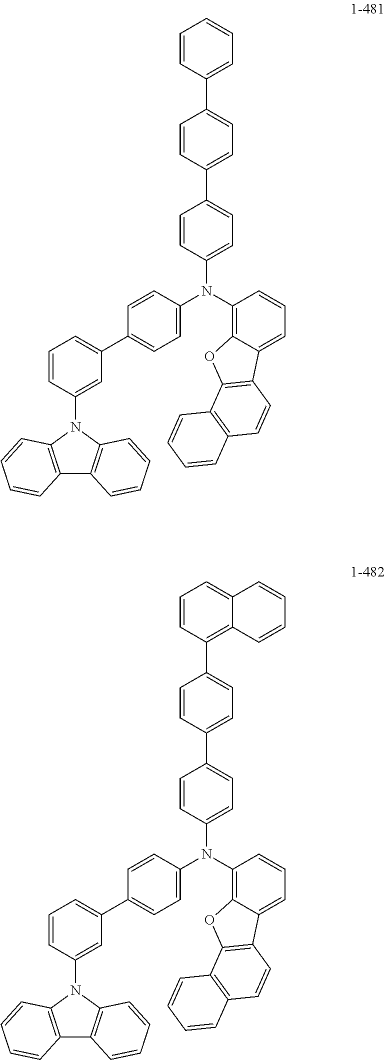 [SMILES: c1ccc(-c2ccc(-c3ccc(N(c4ccc(-c5cccc(-n6c7ccccc7c7ccccc76)c5)cc4)c4cccc5c4oc4c6ccccc6ccc54)cc3)cc2)cc1 ; c1cc(-c2ccc(N(c3ccc(-c4ccc(-c5cccc6ccccc56)cc4)cc3)c3cccc4c3oc3c5ccccc5ccc43)cc2)cc(-n2c3ccccc3c3ccccc32)c1]

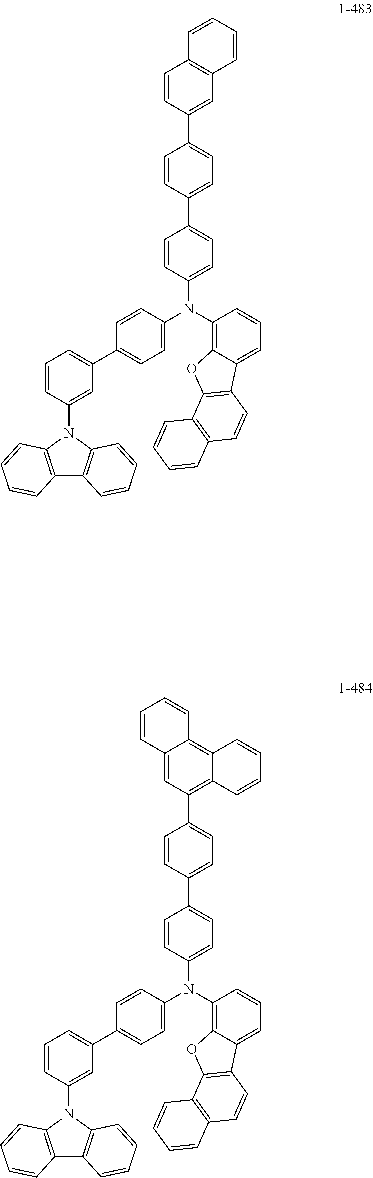 [SMILES: c1cc(-c2ccc(N(c3ccc(-c4ccc(-c5ccc6ccccc6c5)cc4)cc3)c3cccc4c3oc3c5ccccc5ccc43)cc2)cc(-n2c3ccccc3c3ccccc32)c1 ; c1cc(-c2ccc(N(c3ccc(-c4ccc(-c5cc6ccccc6c6ccccc56)cc4)cc3)c3cccc4c3oc3c5ccccc5ccc43)cc2)cc(-n2c3ccccc3c3ccccc32)c1]

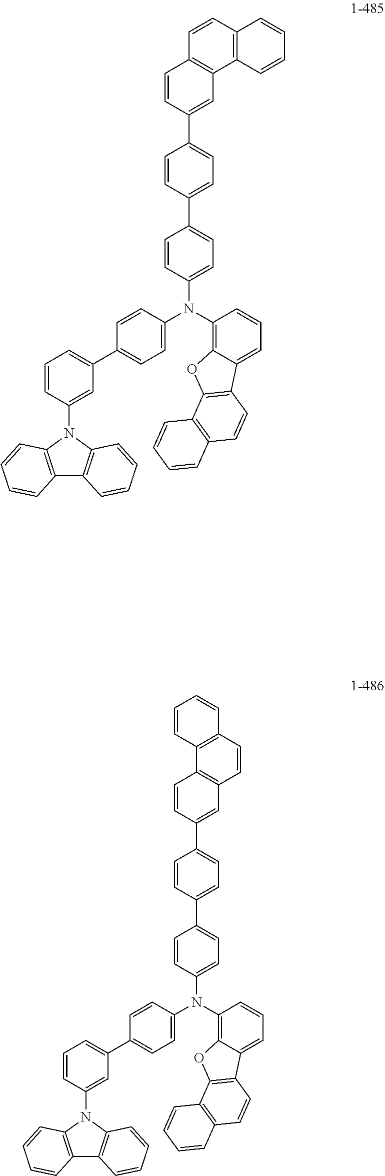 [SMILES: c1cc(-c2ccc(N(c3ccc(-c4ccc(-c5ccc6ccc7ccccc7c6c5)cc4)cc3)c3cccc4c3oc3c5ccccc5ccc43)cc2)cc(-n2c3ccccc3c3ccccc32)c1 ; c1cc(-c2ccc(N(c3ccc(-c4ccc(-c5ccc6c(ccc7ccccc76)c5)cc4)cc3)c3cccc4c3oc3c5ccccc5ccc43)cc2)cc(-n2c3ccccc3c3ccccc32)c1]

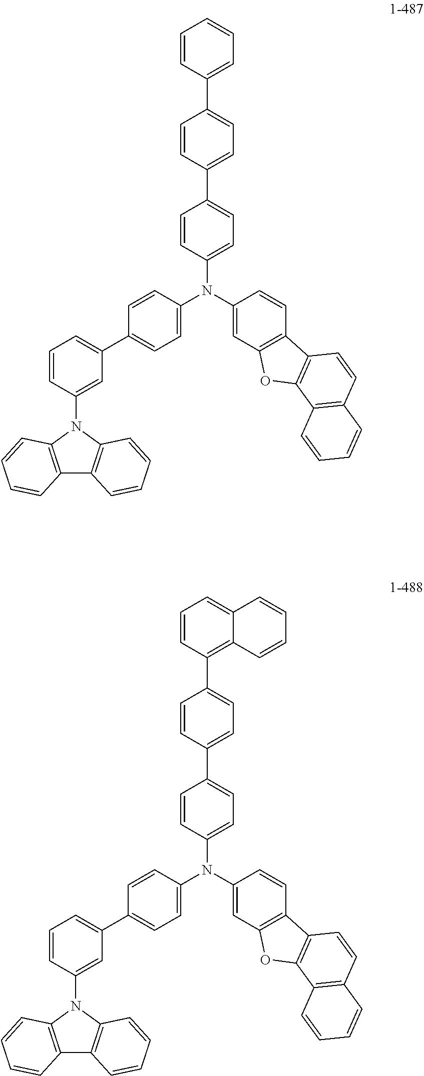 [SMILES: c1ccc(-c2ccc(-c3ccc(N(c4ccc(-c5cccc(-n6c7ccccc7c7ccccc76)c5)cc4)c4ccc5c(c4)oc4c6ccccc6ccc54)cc3)cc2)cc1 ; c1cc(-c2ccc(N(c3ccc(-c4ccc(-c5cccc6ccccc56)cc4)cc3)c3ccc4c(c3)oc3c5ccccc5ccc43)cc2)cc(-n2c3ccccc3c3ccccc32)c1]

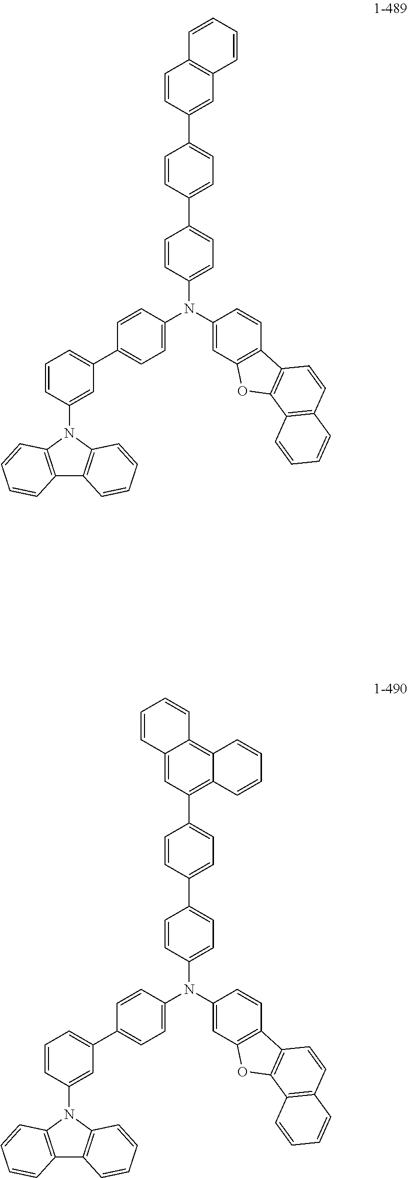 [SMILES: c1cc(-c2ccc(N(c3ccc(-c4ccc(-c5ccc6ccccc6c5)cc4)cc3)c3ccc4c(c3)oc3c5ccccc5ccc43)cc2)cc(-n2c3ccccc3c3ccccc32)c1 ; c1cc(-c2ccc(N(c3ccc(-c4ccc(-c5cc6ccccc6c6ccccc56)cc4)cc3)c3ccc4c(c3)oc3c5ccccc5ccc43)cc2)cc(-n2c3ccccc3c3ccccc32)c1]

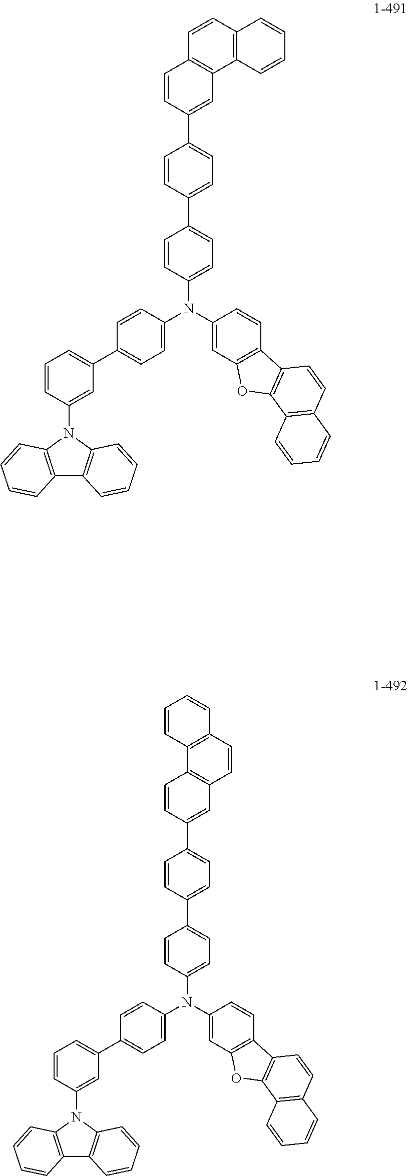 [SMILES: c1cc(-c2ccc(N(c3ccc(-c4ccc(-c5ccc6ccc7ccccc7c6c5)cc4)cc3)c3ccc4c(c3)oc3c5ccccc5ccc43)cc2)cc(-n2c3ccccc3c3ccccc32)c1 ; c1cc(-c2ccc(N(c3ccc(-c4ccc(-c5ccc6c(ccc7ccccc76)c5)cc4)cc3)c3ccc4c(c3)oc3c5ccccc5ccc43)cc2)cc(-n2c3ccccc3c3ccccc32)c1]

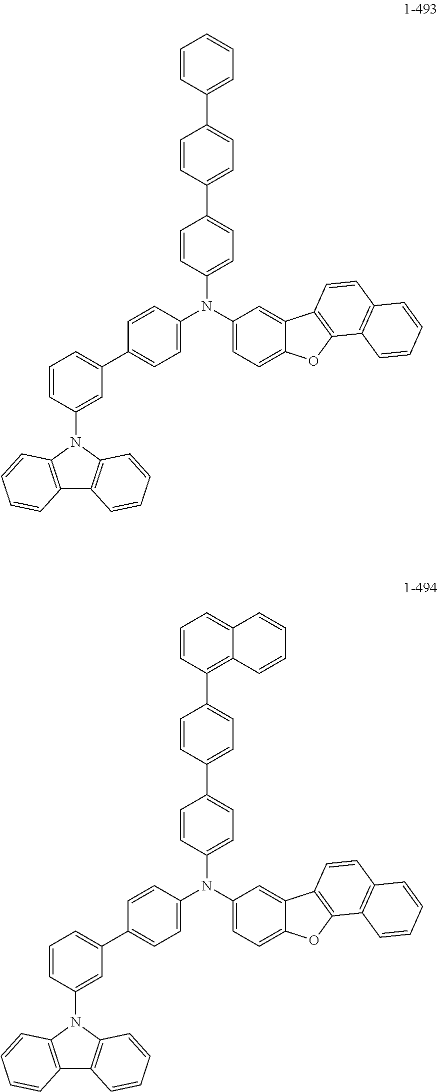 [SMILES: c1ccc(-c2ccc(-c3ccc(N(c4ccc(-c5cccc(-n6c7ccccc7c7ccccc76)c5)cc4)c4ccc5oc6c7ccccc7ccc6c5c4)cc3)cc2)cc1 ; c1cc(-c2ccc(N(c3ccc(-c4ccc(-c5cccc6ccccc56)cc4)cc3)c3ccc4oc5c6ccccc6ccc5c4c3)cc2)cc(-n2c3ccccc3c3ccccc32)c1]

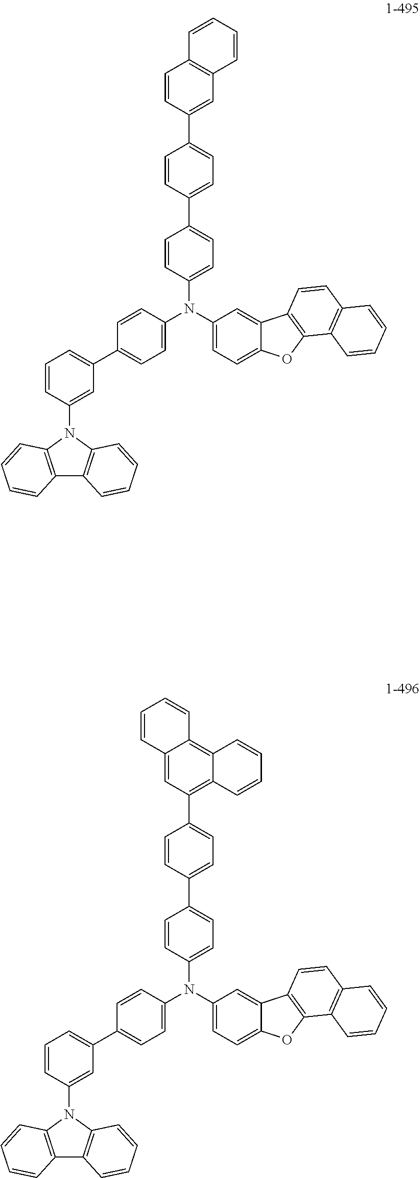 [SMILES: c1cc(-c2ccc(N(c3ccc(-c4ccc(-c5ccc6ccccc6c5)cc4)cc3)c3ccc4oc5c6ccccc6ccc5c4c3)cc2)cc(-n2c3ccccc3c3ccccc32)c1 ; c1cc(-c2ccc(N(c3ccc(-c4ccc(-c5cc6ccccc6c6ccccc56)cc4)cc3)c3ccc4oc5c6ccccc6ccc5c4c3)cc2)cc(-n2c3ccccc3c3ccccc32)c1]

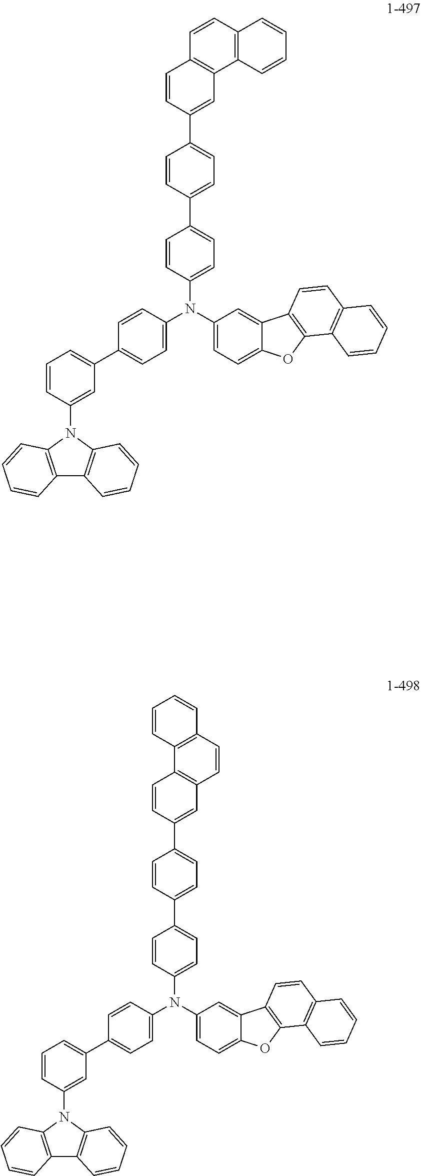 [SMILES: c1cc(-c2ccc(N(c3ccc(-c4ccc(-c5ccc6ccc7ccccc7c6c5)cc4)cc3)c3ccc4oc5c6ccccc6ccc5c4c3)cc2)cc(-n2c3ccccc3c3ccccc32)c1 ; c1cc(-c2ccc(N(c3ccc(-c4ccc(-c5ccc6c(ccc7ccccc76)c5)cc4)cc3)c3ccc4oc5c6ccccc6ccc5c4c3)cc2)cc(-n2c3ccccc3c3ccccc32)c1]

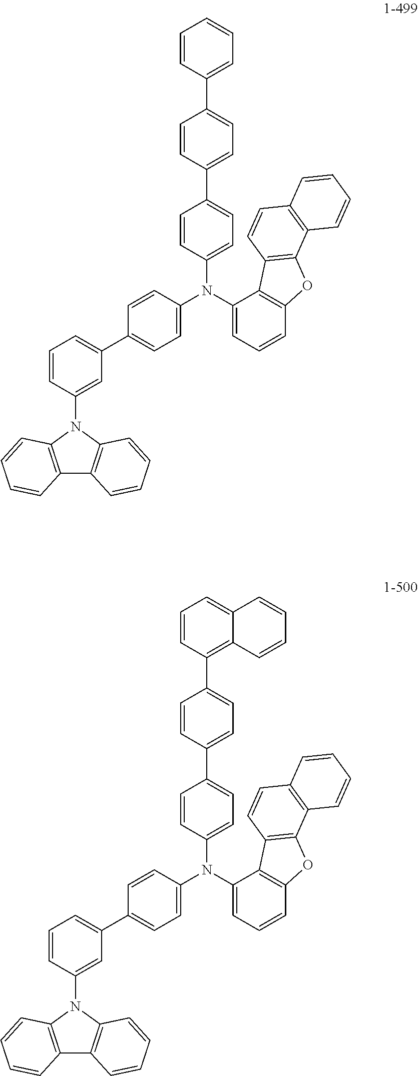 [SMILES: c1ccc(-c2ccc(-c3ccc(N(c4ccc(-c5cccc(-n6c7ccccc7c7ccccc76)c5)cc4)c4cccc5oc6c7ccccc7ccc6c45)cc3)cc2)cc1 ; c1cc(-c2ccc(N(c3ccc(-c4ccc(-c5cccc6ccccc56)cc4)cc3)c3cccc4oc5c6ccccc6ccc5c34)cc2)cc(-n2c3ccccc3c3ccccc32)c1]

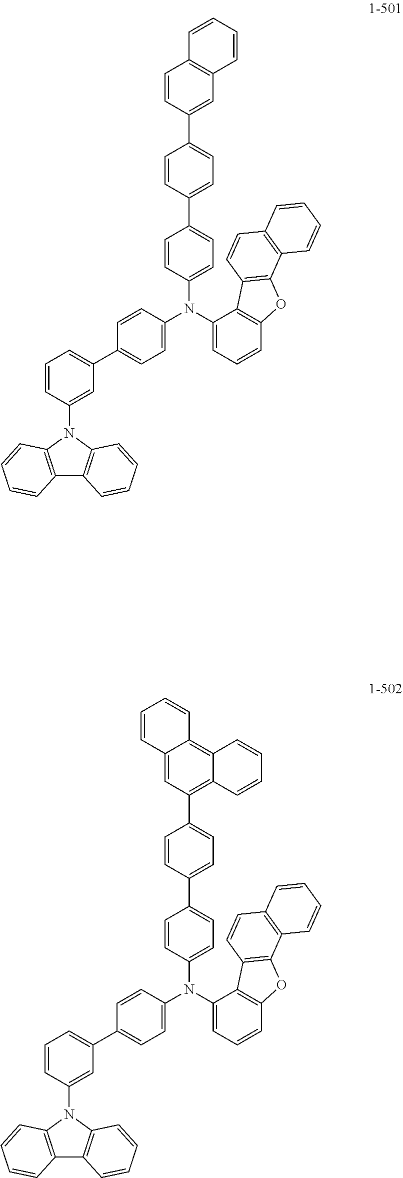 [SMILES: c1cc(-c2ccc(N(c3ccc(-c4ccc(-c5ccc6ccccc6c5)cc4)cc3)c3cccc4oc5c6ccccc6ccc5c34)cc2)cc(-n2c3ccccc3c3ccccc32)c1 ; c1cc(-c2ccc(N(c3ccc(-c4ccc(-c5cc6ccccc6c6ccccc56)cc4)cc3)c3cccc4oc5c6ccccc6ccc5c34)cc2)cc(-n2c3ccccc3c3ccccc32)c1]

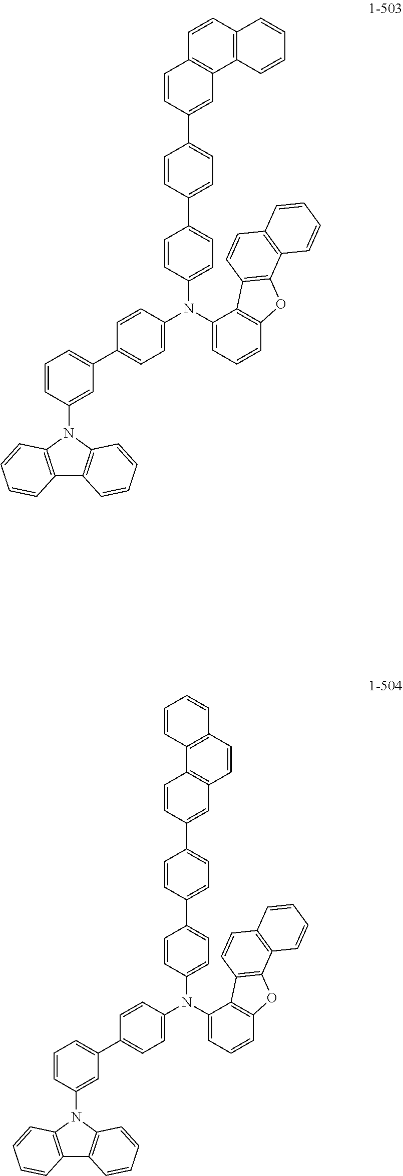 [SMILES: c1cc(-c2ccc(N(c3ccc(-c4ccc(-c5ccc6ccc7ccccc7c6c5)cc4)cc3)c3cccc4oc5c6ccccc6ccc5c34)cc2)cc(-n2c3ccccc3c3ccccc32)c1 ; c1cc(-c2ccc(N(c3ccc(-c4ccc(-c5ccc6c(ccc7ccccc76)c5)cc4)cc3)c3cccc4oc5c6ccccc6ccc5c34)cc2)cc(-n2c3ccccc3c3ccccc32)c1]

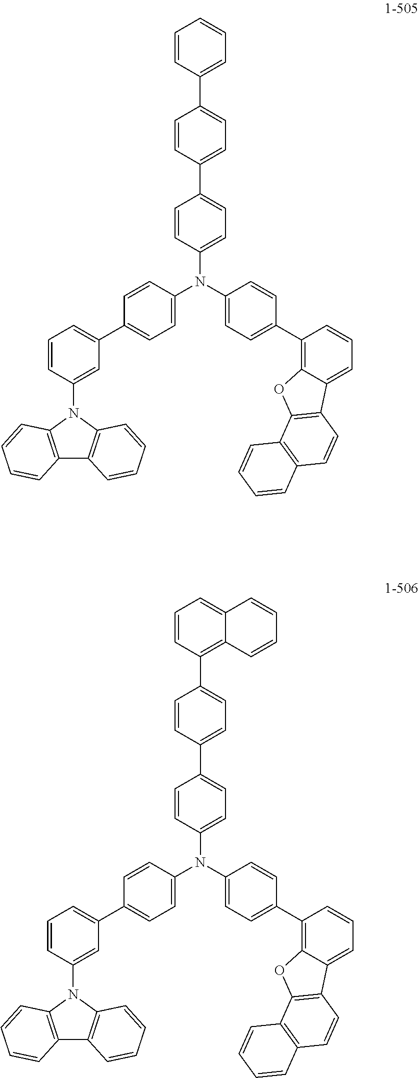 [SMILES: c1ccc(-c2ccc(-c3ccc(N(c4ccc(-c5cccc(-n6c7ccccc7c7ccccc76)c5)cc4)c4ccc(-c5cccc6c5oc5c7ccccc7ccc65)cc4)cc3)cc2)cc1 ; c1cc(-c2ccc(N(c3ccc(-c4ccc(-c5cccc6ccccc56)cc4)cc3)c3ccc(-c4cccc5c4oc4c6ccccc6ccc54)cc3)cc2)cc(-n2c3ccccc3c3ccccc32)c1]

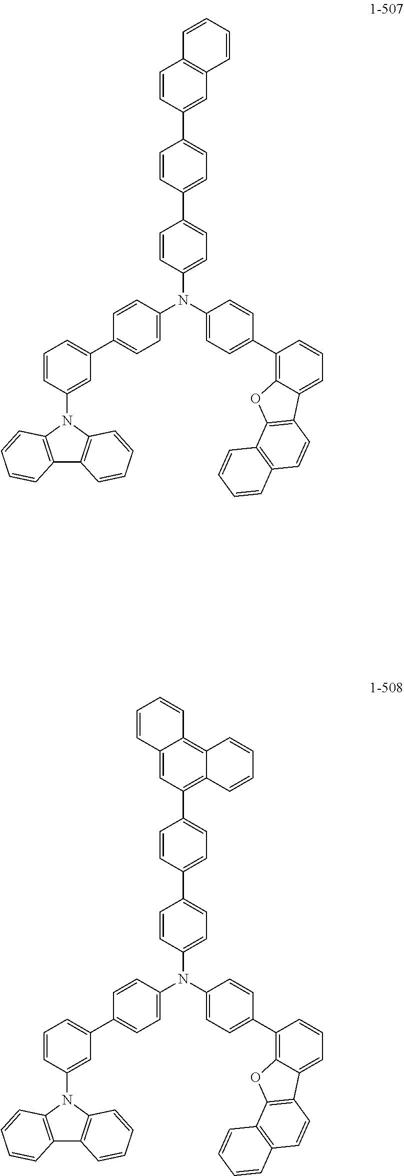 [SMILES: c1cc(-c2ccc(N(c3ccc(-c4ccc(-c5ccc6ccccc6c5)cc4)cc3)c3ccc(-c4cccc5c4oc4c6ccccc6ccc54)cc3)cc2)cc(-n2c3ccccc3c3ccccc32)c1 ; c1cc(-c2ccc(N(c3ccc(-c4ccc(-c5cc6ccccc6c6ccccc56)cc4)cc3)c3ccc(-c4cccc5c4oc4c6ccccc6ccc54)cc3)cc2)cc(-n2c3ccccc3c3ccccc32)c1]

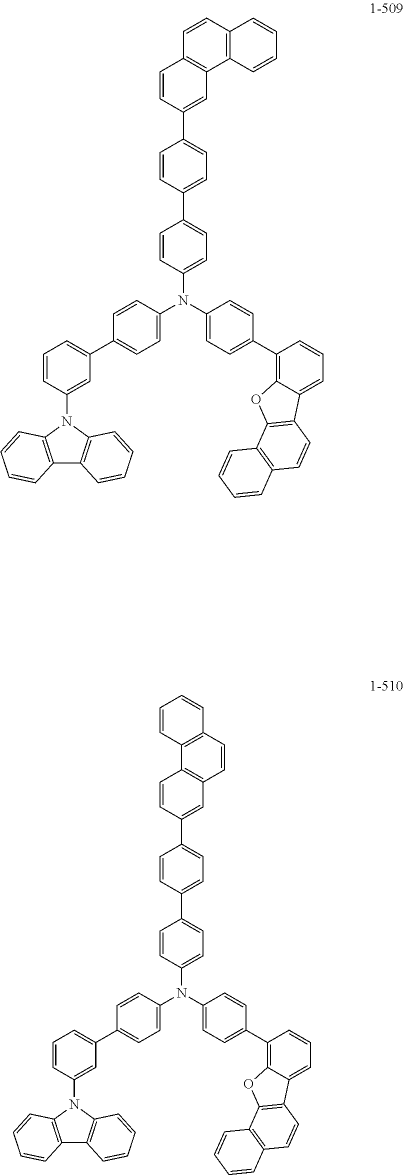 [SMILES: c1cc(-c2ccc(N(c3ccc(-c4ccc(-c5ccc6ccc7ccccc7c6c5)cc4)cc3)c3ccc(-c4cccc5c4oc4c6ccccc6ccc54)cc3)cc2)cc(-n2c3ccccc3c3ccccc32)c1 ; c1cc(-c2ccc(N(c3ccc(-c4ccc(-c5ccc6c(ccc7ccccc76)c5)cc4)cc3)c3ccc(-c4cccc5c4oc4c6ccccc6ccc54)cc3)cc2)cc(-n2c3ccccc3c3ccccc32)c1]

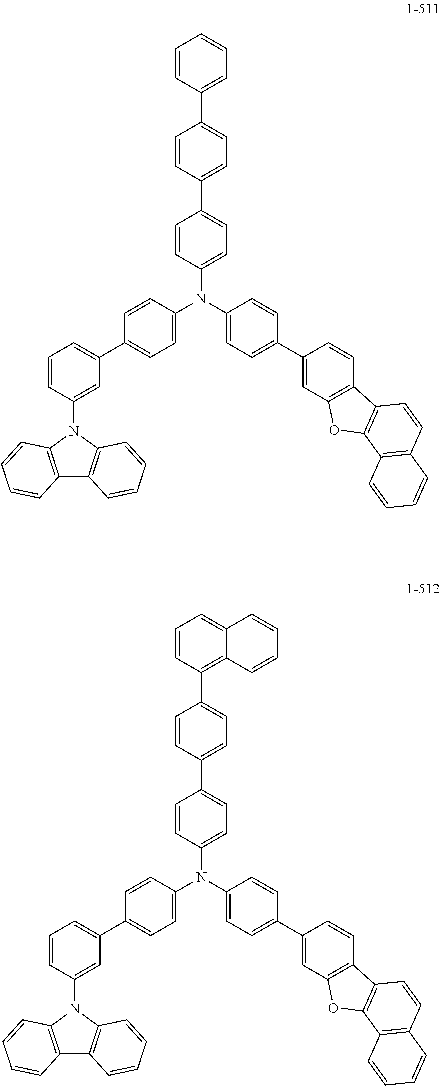 [SMILES: c1ccc(-c2ccc(-c3ccc(N(c4ccc(-c5cccc(-n6c7ccccc7c7ccccc76)c5)cc4)c4ccc(-c5ccc6c(c5)oc5c7ccccc7ccc65)cc4)cc3)cc2)cc1 ; c1cc(-c2ccc(N(c3ccc(-c4ccc(-c5cccc6ccccc56)cc4)cc3)c3ccc(-c4ccc5c(c4)oc4c6ccccc6ccc54)cc3)cc2)cc(-n2c3ccccc3c3ccccc32)c1]

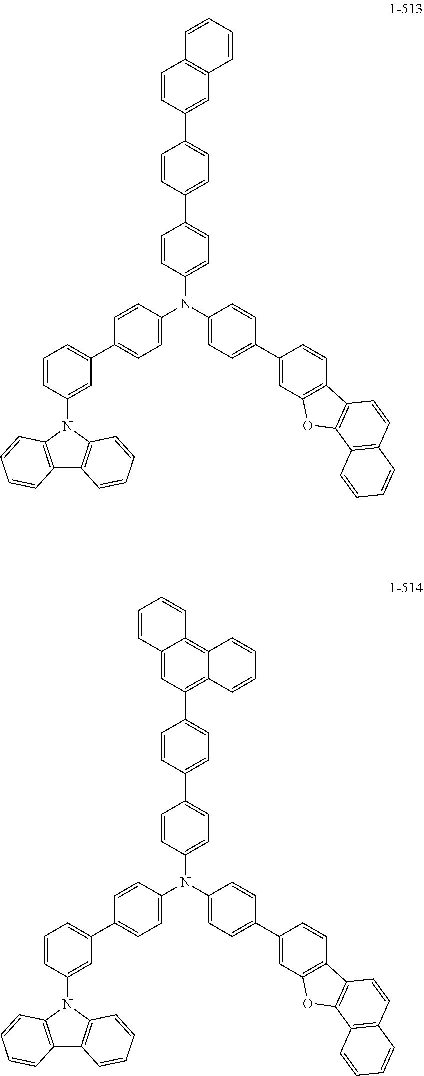 [SMILES: c1cc(-c2ccc(N(c3ccc(-c4ccc(-c5ccc6ccccc6c5)cc4)cc3)c3ccc(-c4ccc5c(c4)oc4c6ccccc6ccc54)cc3)cc2)cc(-n2c3ccccc3c3ccccc32)c1 ; c1cc(-c2ccc(N(c3ccc(-c4ccc(-c5cc6ccccc6c6ccccc56)cc4)cc3)c3ccc(-c4ccc5c(c4)oc4c6ccccc6ccc54)cc3)cc2)cc(-n2c3ccccc3c3ccccc32)c1]

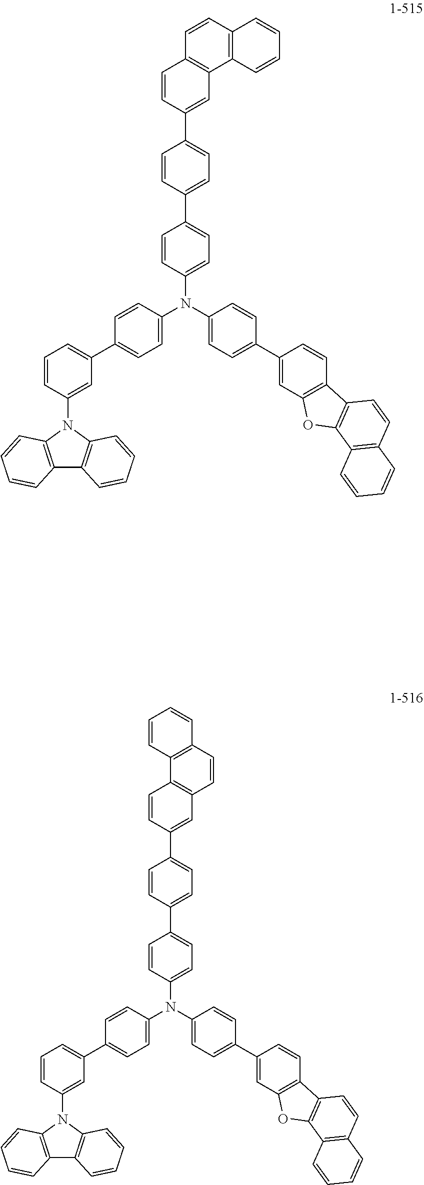 [SMILES: c1cc(-c2ccc(N(c3ccc(-c4ccc(-c5ccc6ccc7ccccc7c6c5)cc4)cc3)c3ccc(-c4ccc5c(c4)oc4c6ccccc6ccc54)cc3)cc2)cc(-n2c3ccccc3c3ccccc32)c1 ; c1cc(-c2ccc(N(c3ccc(-c4ccc(-c5ccc6c(ccc7ccccc76)c5)cc4)cc3)c3ccc(-c4ccc5c(c4)oc4c6ccccc6ccc54)cc3)cc2)cc(-n2c3ccccc3c3ccccc32)c1]

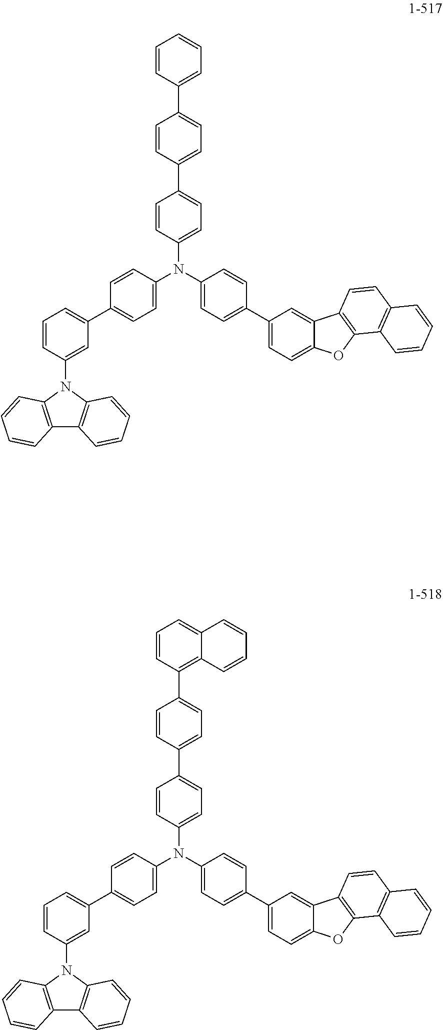 [SMILES: c1ccc(-c2ccc(-c3ccc(N(c4ccc(-c5cccc(-n6c7ccccc7c7ccccc76)c5)cc4)c4ccc(-c5ccc6oc7c8ccccc8ccc7c6c5)cc4)cc3)cc2)cc1 ; c1cc(-c2ccc(N(c3ccc(-c4ccc(-c5cccc6ccccc56)cc4)cc3)c3ccc(-c4ccc5oc6c7ccccc7ccc6c5c4)cc3)cc2)cc(-n2c3ccccc3c3ccccc32)c1]

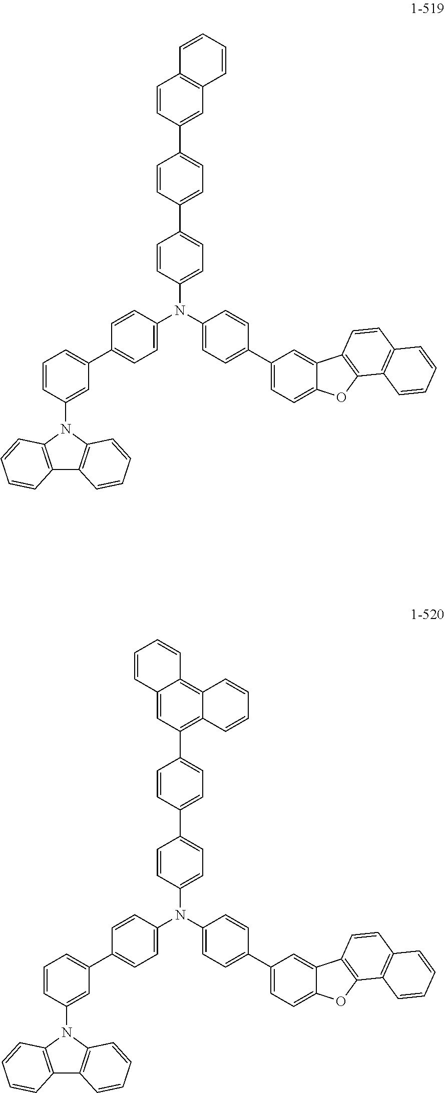 [SMILES: c1cc(-c2ccc(N(c3ccc(-c4ccc(-c5ccc6ccccc6c5)cc4)cc3)c3ccc(-c4ccc5oc6c7ccccc7ccc6c5c4)cc3)cc2)cc(-n2c3ccccc3c3ccccc32)c1 ; c1cc(-c2ccc(N(c3ccc(-c4ccc(-c5cc6ccccc6c6ccccc56)cc4)cc3)c3ccc(-c4ccc5oc6c7ccccc7ccc6c5c4)cc3)cc2)cc(-n2c3ccccc3c3ccccc32)c1]

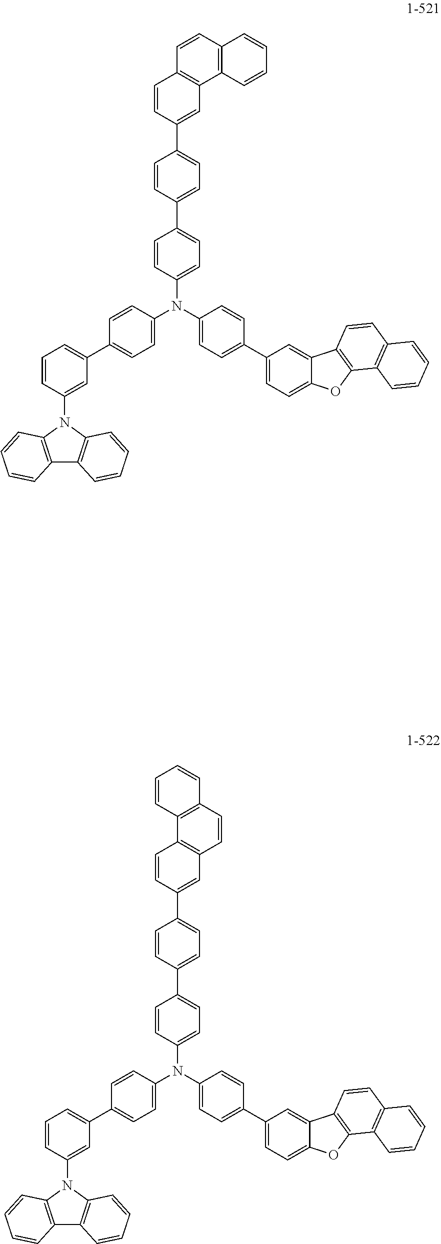 [SMILES: c1cc(-c2ccc(N(c3ccc(-c4ccc(-c5ccc6ccc7ccccc7c6c5)cc4)cc3)c3ccc(-c4ccc5oc6c7ccccc7ccc6c5c4)cc3)cc2)cc(-n2c3ccccc3c3ccccc32)c1 ; c1cc(-c2ccc(N(c3ccc(-c4ccc(-c5ccc6c(ccc7ccccc76)c5)cc4)cc3)c3ccc(-c4ccc5oc6c7ccccc7ccc6c5c4)cc3)cc2)cc(-n2c3ccccc3c3ccccc32)c1]

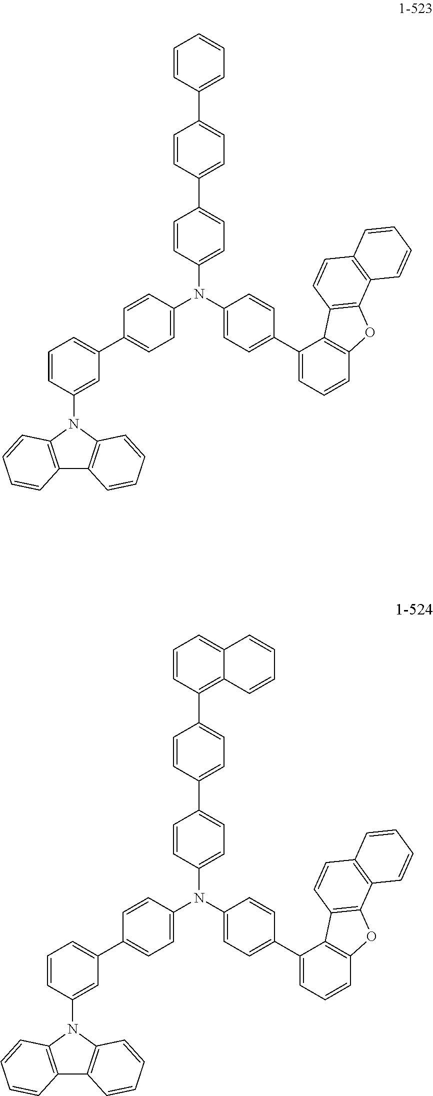 [SMILES: c1ccc(-c2ccc(-c3ccc(N(c4ccc(-c5cccc(-n6c7ccccc7c7ccccc76)c5)cc4)c4ccc(-c5cccc6oc7c8ccccc8ccc7c56)cc4)cc3)cc2)cc1 ; c1cc(-c2ccc(N(c3ccc(-c4ccc(-c5cccc6ccccc56)cc4)cc3)c3ccc(-c4cccc5oc6c7ccccc7ccc6c45)cc3)cc2)cc(-n2c3ccccc3c3ccccc32)c1]

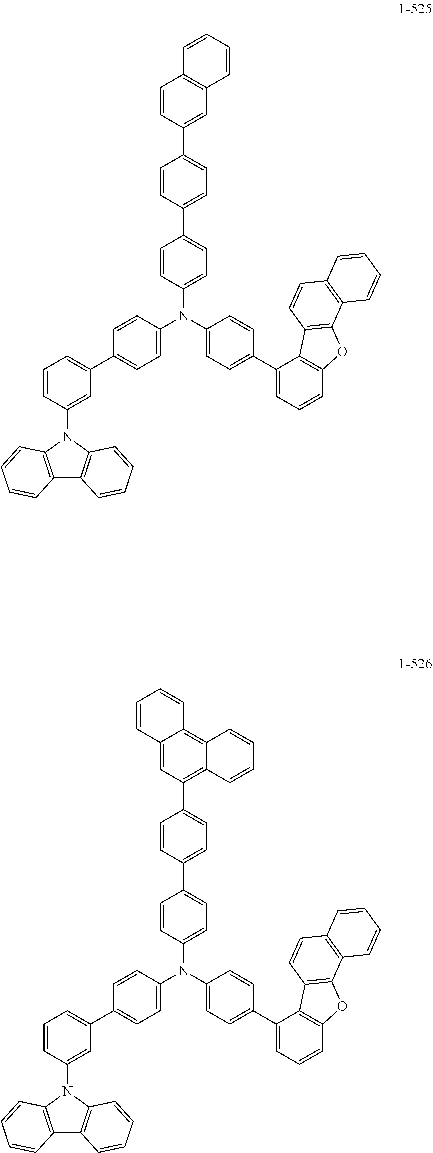 [SMILES: c1cc(-c2ccc(N(c3ccc(-c4ccc(-c5ccc6ccccc6c5)cc4)cc3)c3ccc(-c4cccc5oc6c7ccccc7ccc6c45)cc3)cc2)cc(-n2c3ccccc3c3ccccc32)c1 ; c1cc(-c2ccc(N(c3ccc(-c4ccc(-c5cc6ccccc6c6ccccc56)cc4)cc3)c3ccc(-c4cccc5oc6c7ccccc7ccc6c45)cc3)cc2)cc(-n2c3ccccc3c3ccccc32)c1]

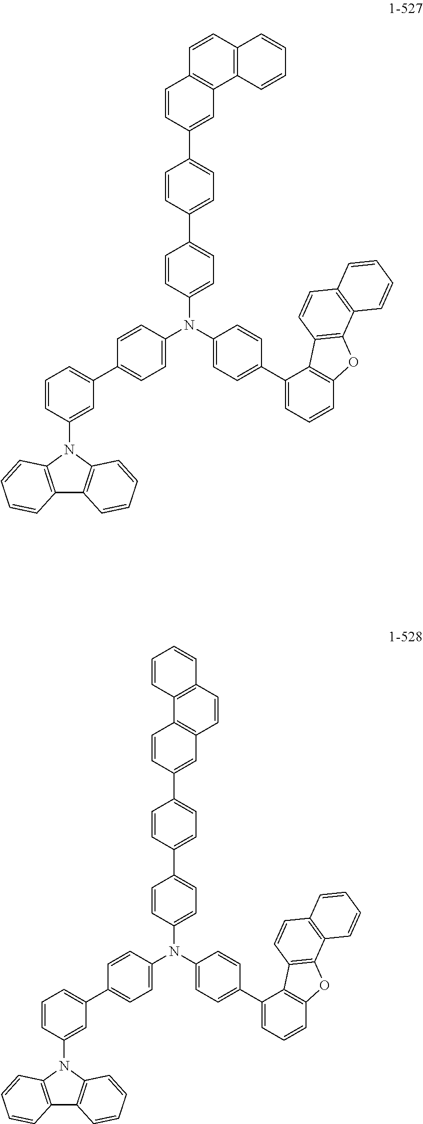 [SMILES: c1cc(-c2ccc(N(c3ccc(-c4ccc(-c5ccc6ccc7ccccc7c6c5)cc4)cc3)c3ccc(-c4cccc5oc6c7ccccc7ccc6c45)cc3)cc2)cc(-n2c3ccccc3c3ccccc32)c1 ; c1cc(-c2ccc(N(c3ccc(-c4ccc(-c5ccc6c(ccc7ccccc76)c5)cc4)cc3)c3ccc(-c4cccc5oc6c7ccccc7ccc6c45)cc3)cc2)cc(-n2c3ccccc3c3ccccc32)c1]

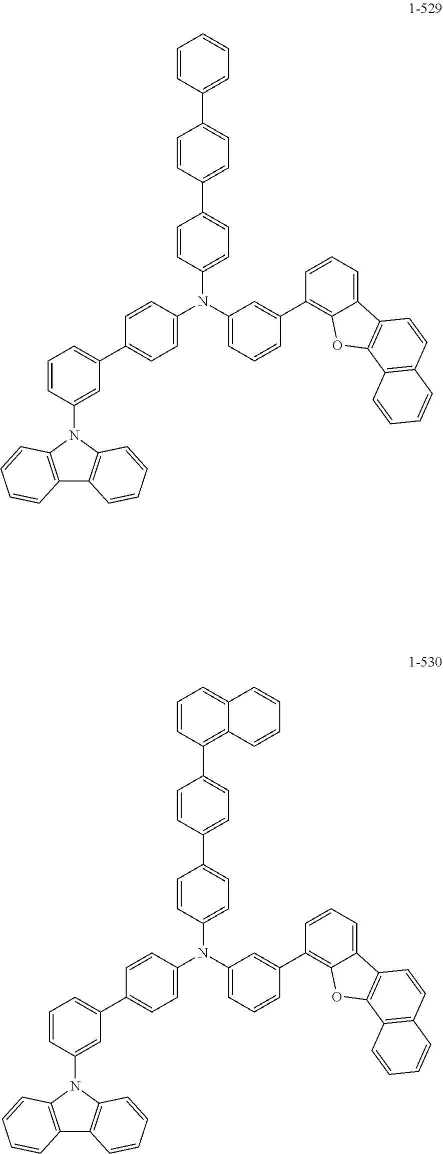 [SMILES: c1ccc(-c2ccc(-c3ccc(N(c4ccc(-c5cccc(-n6c7ccccc7c7ccccc76)c5)cc4)c4cccc(-c5cccc6c5oc5c7ccccc7ccc65)c4)cc3)cc2)cc1 ; c1cc(-c2cccc3c2oc2c4ccccc4ccc32)cc(N(c2ccc(-c3ccc(-c4cccc5ccccc45)cc3)cc2)c2ccc(-c3cccc(-n4c5ccccc5c5ccccc54)c3)cc2)c1]

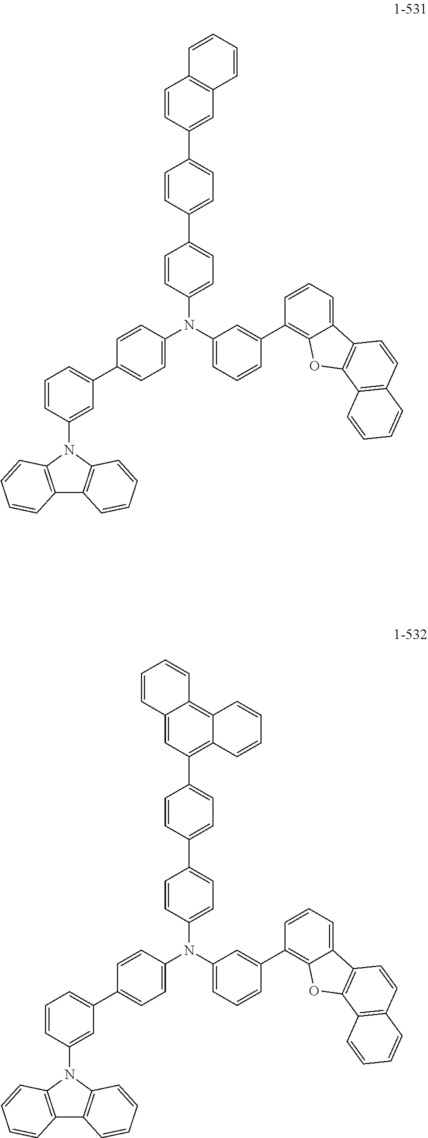 [SMILES: c1cc(-c2cccc3c2oc2c4ccccc4ccc32)cc(N(c2ccc(-c3ccc(-c4ccc5ccccc5c4)cc3)cc2)c2ccc(-c3cccc(-n4c5ccccc5c5ccccc54)c3)cc2)c1 ; c1cc(-c2cccc3c2oc2c4ccccc4ccc32)cc(N(c2ccc(-c3ccc(-c4cc5ccccc5c5ccccc45)cc3)cc2)c2ccc(-c3cccc(-n4c5ccccc5c5ccccc54)c3)cc2)c1]

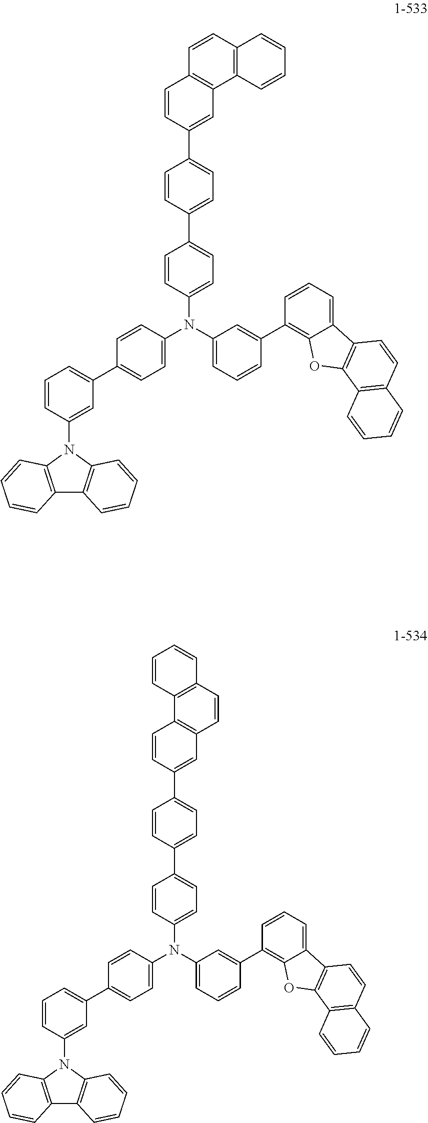 [SMILES: c1cc(-c2cccc3c2oc2c4ccccc4ccc32)cc(N(c2ccc(-c3ccc(-c4ccc5ccc6ccccc6c5c4)cc3)cc2)c2ccc(-c3cccc(-n4c5ccccc5c5ccccc54)c3)cc2)c1 ; c1cc(-c2cccc3c2oc2c4ccccc4ccc32)cc(N(c2ccc(-c3ccc(-c4ccc5c(ccc6ccccc65)c4)cc3)cc2)c2ccc(-c3cccc(-n4c5ccccc5c5ccccc54)c3)cc2)c1]

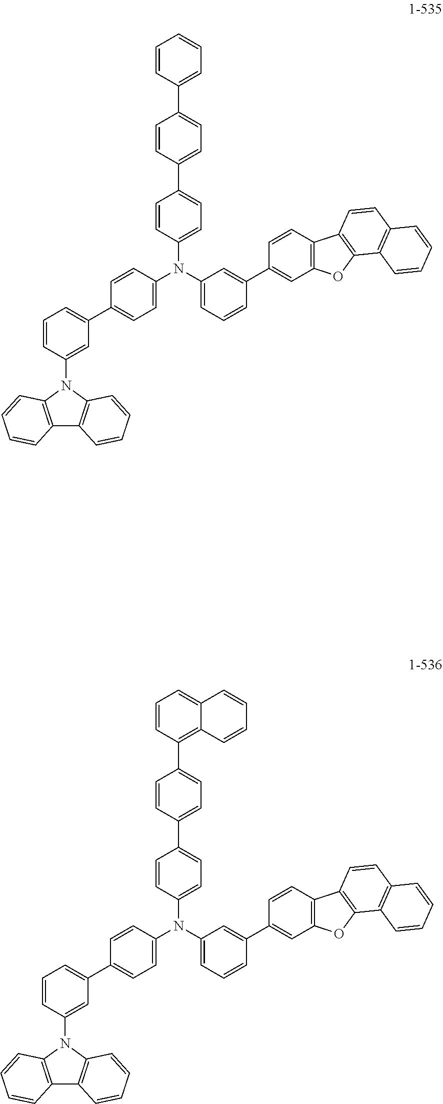 [SMILES: c1ccc(-c2ccc(-c3ccc(N(c4ccc(-c5cccc(-n6c7ccccc7c7ccccc76)c5)cc4)c4cccc(-c5ccc6c(c5)oc5c7ccccc7ccc65)c4)cc3)cc2)cc1 ; c1cc(-c2ccc3c(c2)oc2c4ccccc4ccc32)cc(N(c2ccc(-c3ccc(-c4cccc5ccccc45)cc3)cc2)c2ccc(-c3cccc(-n4c5ccccc5c5ccccc54)c3)cc2)c1]

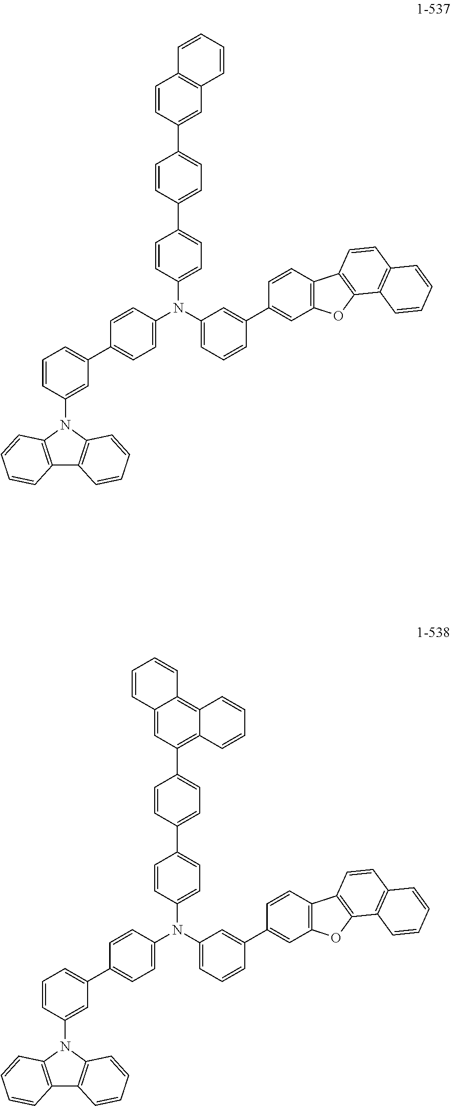 [SMILES: c1cc(-c2ccc3c(c2)oc2c4ccccc4ccc32)cc(N(c2ccc(-c3ccc(-c4ccc5ccccc5c4)cc3)cc2)c2ccc(-c3cccc(-n4c5ccccc5c5ccccc54)c3)cc2)c1 ; c1cc(-c2ccc3c(c2)oc2c4ccccc4ccc32)cc(N(c2ccc(-c3ccc(-c4cc5ccccc5c5ccccc45)cc3)cc2)c2ccc(-c3cccc(-n4c5ccccc5c5ccccc54)c3)cc2)c1]

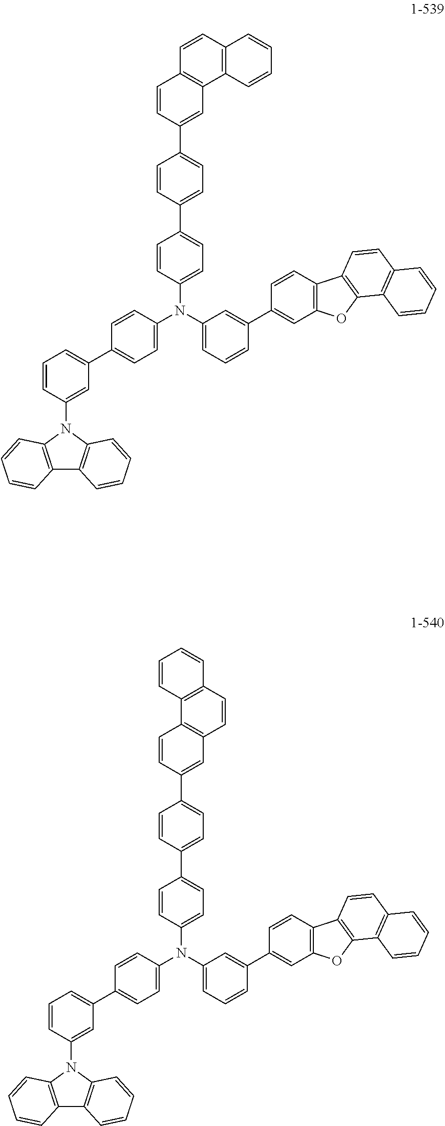 [SMILES: c1cc(-c2ccc3c(c2)oc2c4ccccc4ccc32)cc(N(c2ccc(-c3ccc(-c4ccc5ccc6ccccc6c5c4)cc3)cc2)c2ccc(-c3cccc(-n4c5ccccc5c5ccccc54)c3)cc2)c1 ; c1cc(-c2ccc3c(c2)oc2c4ccccc4ccc32)cc(N(c2ccc(-c3ccc(-c4ccc5c(ccc6ccccc65)c4)cc3)cc2)c2ccc(-c3cccc(-n4c5ccccc5c5ccccc54)c3)cc2)c1]

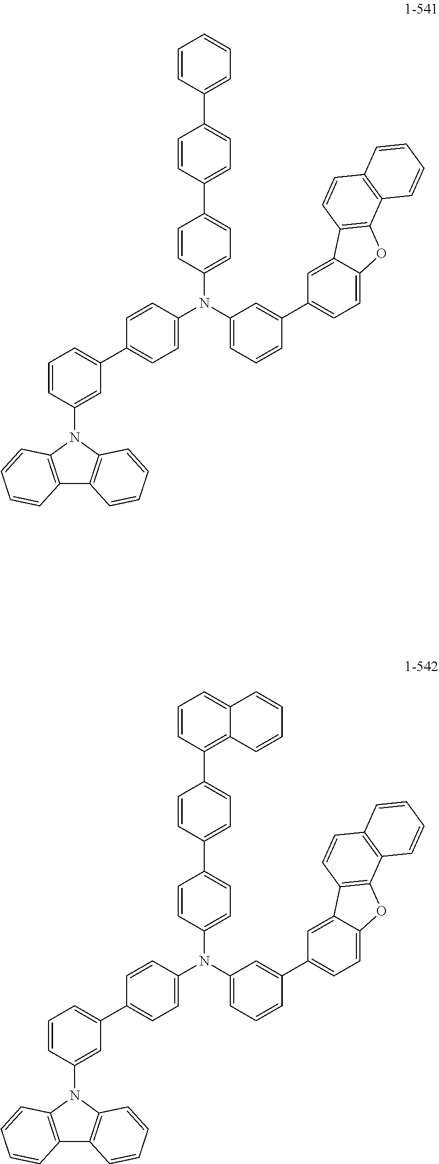 [SMILES: c1ccc(-c2ccc(-c3ccc(N(c4ccc(-c5cccc(-n6c7ccccc7c7ccccc76)c5)cc4)c4cccc(-c5ccc6oc7c8ccccc8ccc7c6c5)c4)cc3)cc2)cc1 ; c1cc(-c2ccc3oc4c5ccccc5ccc4c3c2)cc(N(c2ccc(-c3ccc(-c4cccc5ccccc45)cc3)cc2)c2ccc(-c3cccc(-n4c5ccccc5c5ccccc54)c3)cc2)c1]

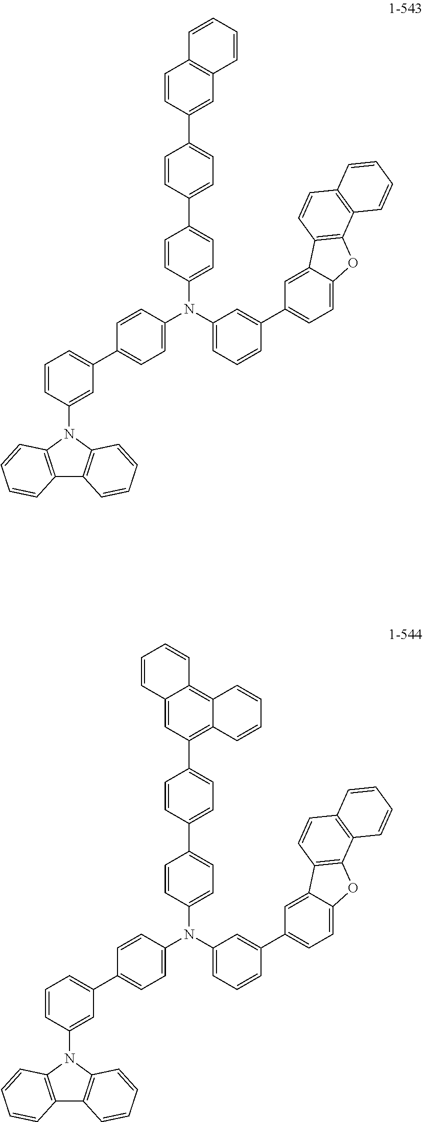 [SMILES: c1cc(-c2ccc3oc4c5ccccc5ccc4c3c2)cc(N(c2ccc(-c3ccc(-c4ccc5ccccc5c4)cc3)cc2)c2ccc(-c3cccc(-n4c5ccccc5c5ccccc54)c3)cc2)c1 ; c1cc(-c2ccc3oc4c5ccccc5ccc4c3c2)cc(N(c2ccc(-c3ccc(-c4cc5ccccc5c5ccccc45)cc3)cc2)c2ccc(-c3cccc(-n4c5ccccc5c5ccccc54)c3)cc2)c1]

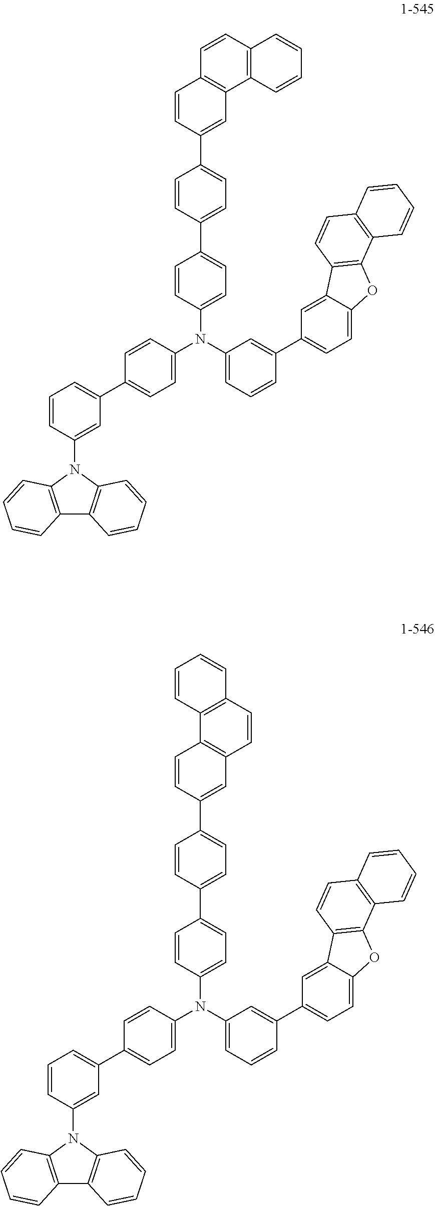 [SMILES: c1cc(-c2ccc3oc4c5ccccc5ccc4c3c2)cc(N(c2ccc(-c3ccc(-c4ccc5ccc6ccccc6c5c4)cc3)cc2)c2ccc(-c3cccc(-n4c5ccccc5c5ccccc54)c3)cc2)c1 ; c1cc(-c2ccc3oc4c5ccccc5ccc4c3c2)cc(N(c2ccc(-c3ccc(-c4ccc5c(ccc6ccccc65)c4)cc3)cc2)c2ccc(-c3cccc(-n4c5ccccc5c5ccccc54)c3)cc2)c1]

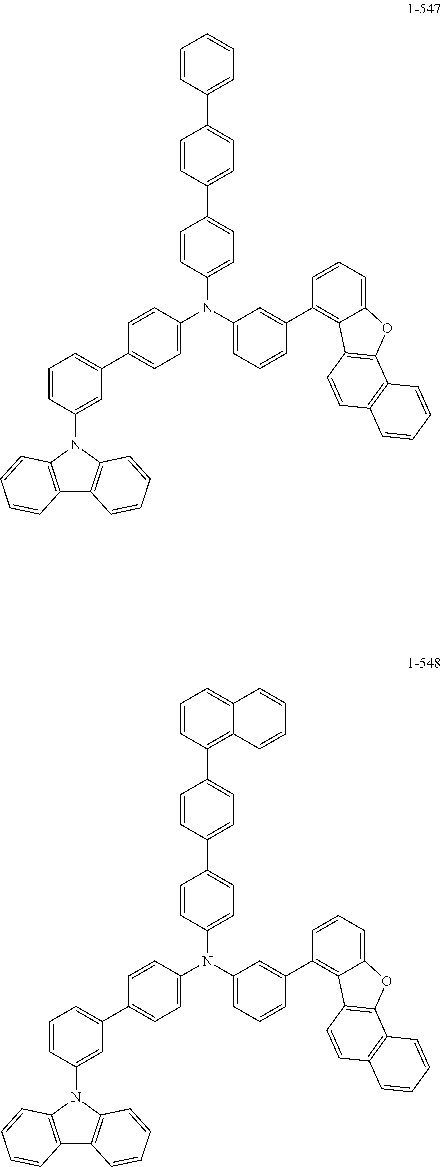 [SMILES: c1ccc(-c2ccc(-c3ccc(N(c4ccc(-c5cccc(-n6c7ccccc7c7ccccc76)c5)cc4)c4cccc(-c5cccc6oc7c8ccccc8ccc7c56)c4)cc3)cc2)cc1 ; c1cc(-c2cccc3oc4c5ccccc5ccc4c23)cc(N(c2ccc(-c3ccc(-c4cccc5ccccc45)cc3)cc2)c2ccc(-c3cccc(-n4c5ccccc5c5ccccc54)c3)cc2)c1]

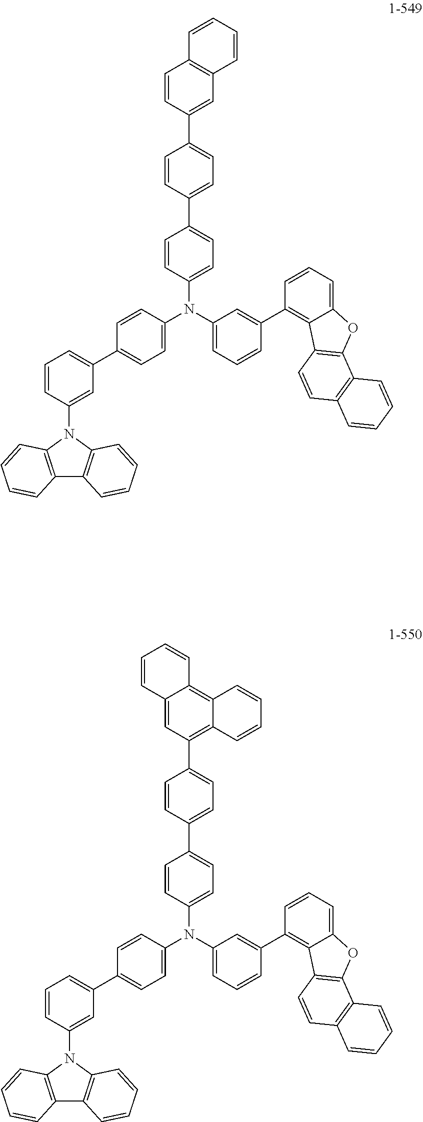 [SMILES: c1cc(-c2cccc3oc4c5ccccc5ccc4c23)cc(N(c2ccc(-c3ccc(-c4ccc5ccccc5c4)cc3)cc2)c2ccc(-c3cccc(-n4c5ccccc5c5ccccc54)c3)cc2)c1 ; c1cc(-c2cccc3oc4c5ccccc5ccc4c23)cc(N(c2ccc(-c3ccc(-c4cc5ccccc5c5ccccc45)cc3)cc2)c2ccc(-c3cccc(-n4c5ccccc5c5ccccc54)c3)cc2)c1]

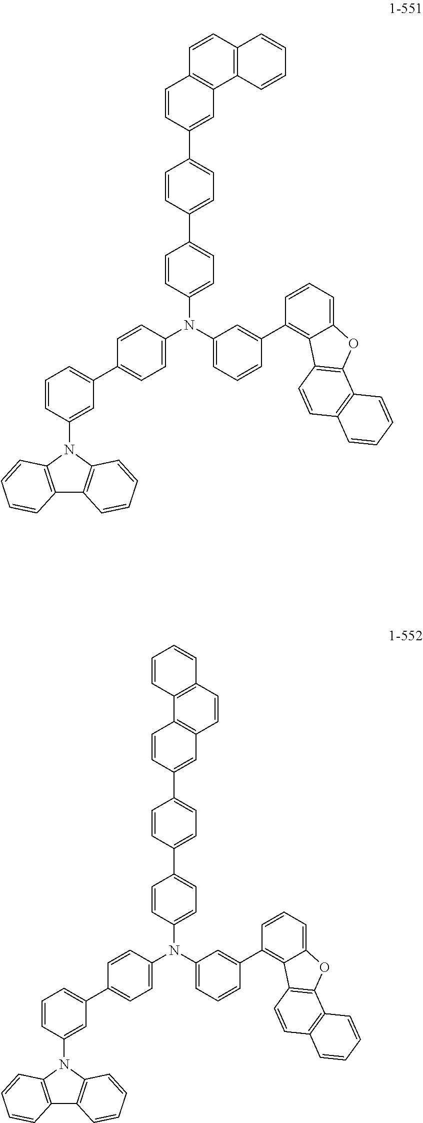 [SMILES: c1cc(-c2cccc3oc4c5ccccc5ccc4c23)cc(N(c2ccc(-c3ccc(-c4ccc5ccc6ccccc6c5c4)cc3)cc2)c2ccc(-c3cccc(-n4c5ccccc5c5ccccc54)c3)cc2)c1 ; c1cc(-c2cccc3oc4c5ccccc5ccc4c23)cc(N(c2ccc(-c3ccc(-c4ccc5c(ccc6ccccc65)c4)cc3)cc2)c2ccc(-c3cccc(-n4c5ccccc5c5ccccc54)c3)cc2)c1]

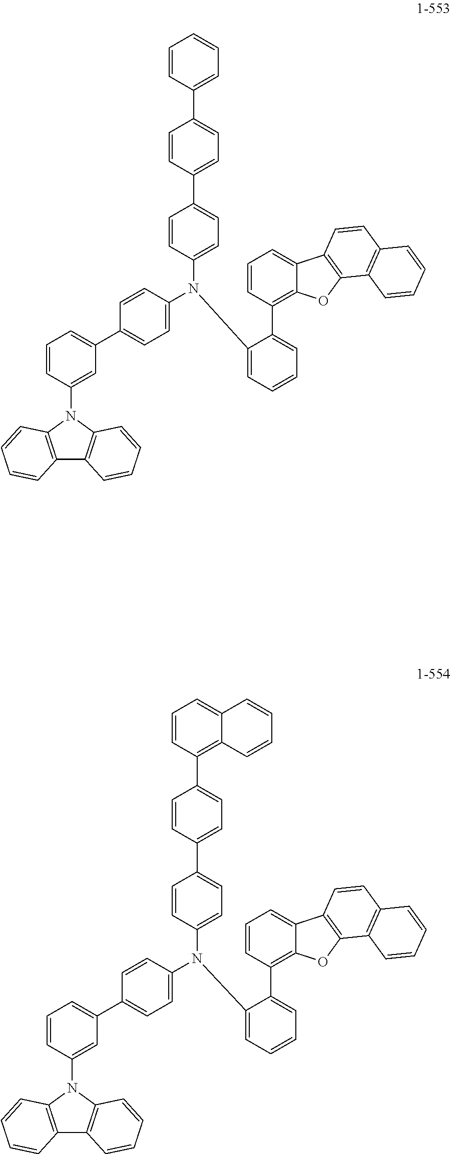 [SMILES: c1ccc(-c2ccc(-c3ccc(N(c4ccc(-c5cccc(-n6c7ccccc7c7ccccc76)c5)cc4)c4ccccc4-c4cccc5c4oc4c6ccccc6ccc54)cc3)cc2)cc1 ; c1cc(-c2ccc(N(c3ccc(-c4ccc(-c5cccc6ccccc56)cc4)cc3)c3ccccc3-c3cccc4c3oc3c5ccccc5ccc43)cc2)cc(-n2c3ccccc3c3ccccc32)c1]

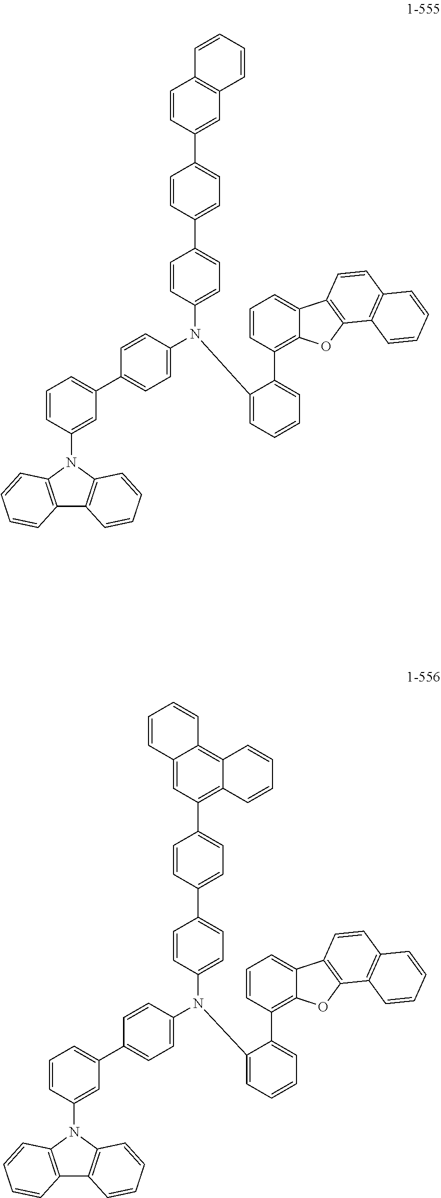 [SMILES: c1cc(-c2ccc(N(c3ccc(-c4ccc(-c5ccc6ccccc6c5)cc4)cc3)c3ccccc3-c3cccc4c3oc3c5ccccc5ccc43)cc2)cc(-n2c3ccccc3c3ccccc32)c1 ; c1cc(-c2ccc(N(c3ccc(-c4ccc(-c5cc6ccccc6c6ccccc56)cc4)cc3)c3ccccc3-c3cccc4c3oc3c5ccccc5ccc43)cc2)cc(-n2c3ccccc3c3ccccc32)c1]

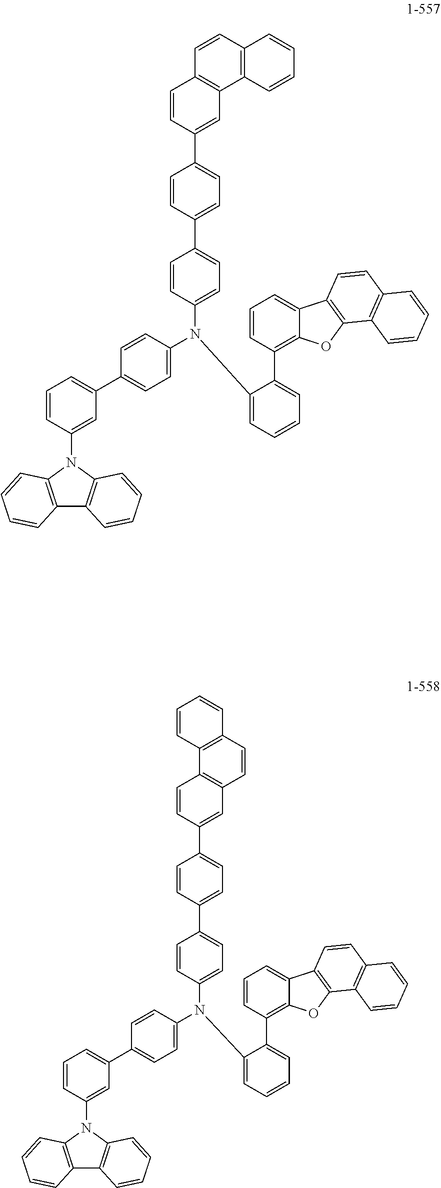 [SMILES: c1cc(-c2ccc(N(c3ccc(-c4ccc(-c5ccc6ccc7ccccc7c6c5)cc4)cc3)c3ccccc3-c3cccc4c3oc3c5ccccc5ccc43)cc2)cc(-n2c3ccccc3c3ccccc32)c1 ; c1cc(-c2ccc(N(c3ccc(-c4ccc(-c5ccc6c(ccc7ccccc76)c5)cc4)cc3)c3ccccc3-c3cccc4c3oc3c5ccccc5ccc43)cc2)cc(-n2c3ccccc3c3ccccc32)c1]

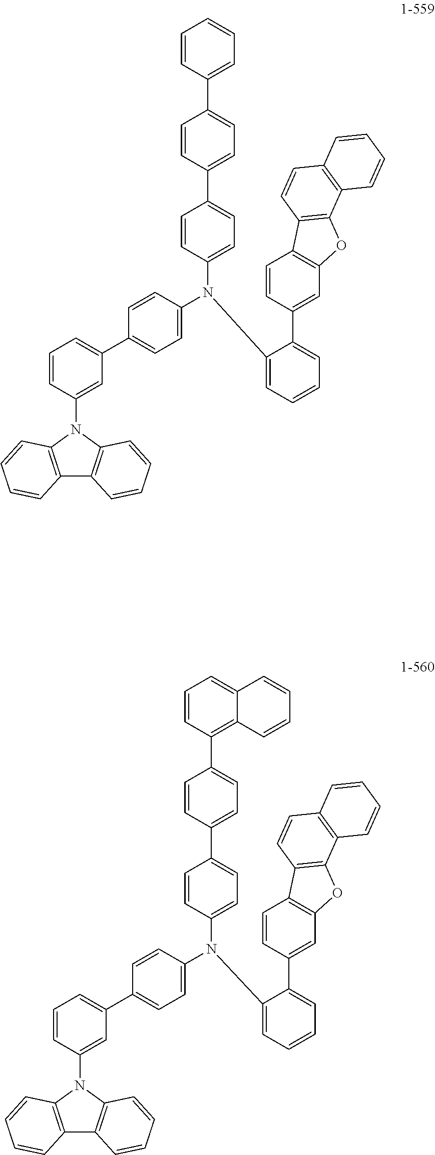 [SMILES: c1ccc(-c2ccc(-c3ccc(N(c4ccc(-c5cccc(-n6c7ccccc7c7ccccc76)c5)cc4)c4ccccc4-c4ccc5c(c4)oc4c6ccccc6ccc54)cc3)cc2)cc1 ; c1cc(-c2ccc(N(c3ccc(-c4ccc(-c5cccc6ccccc56)cc4)cc3)c3ccccc3-c3ccc4c(c3)oc3c5ccccc5ccc43)cc2)cc(-n2c3ccccc3c3ccccc32)c1]

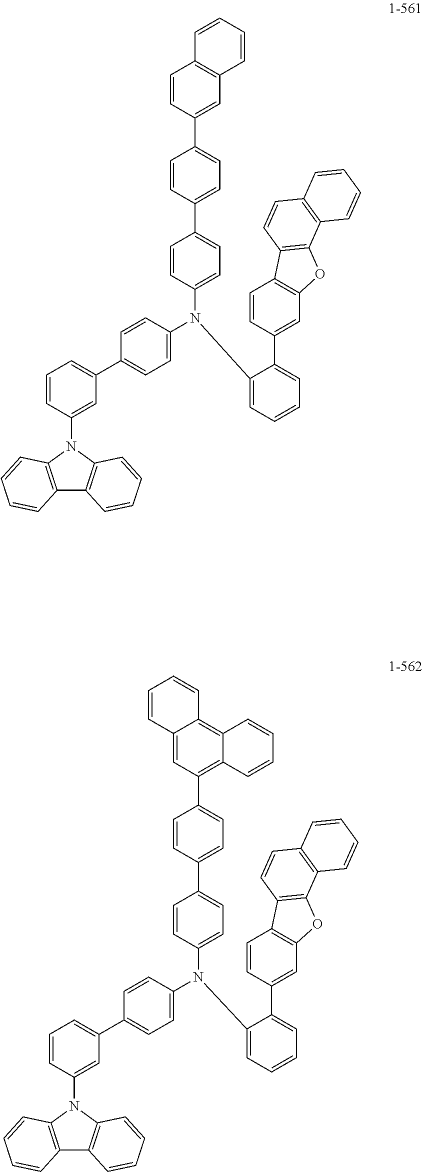 [SMILES: c1cc(-c2ccc(N(c3ccc(-c4ccc(-c5ccc6ccccc6c5)cc4)cc3)c3ccccc3-c3ccc4c(c3)oc3c5ccccc5ccc43)cc2)cc(-n2c3ccccc3c3ccccc32)c1 ; c1cc(-c2ccc(N(c3ccc(-c4ccc(-c5cc6ccccc6c6ccccc56)cc4)cc3)c3ccccc3-c3ccc4c(c3)oc3c5ccccc5ccc43)cc2)cc(-n2c3ccccc3c3ccccc32)c1]

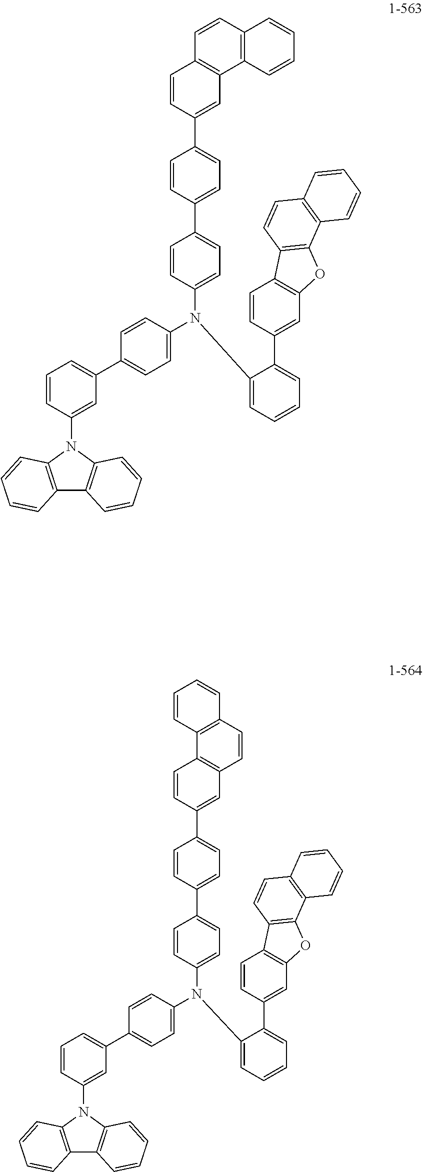 [SMILES: c1cc(-c2ccc(N(c3ccc(-c4ccc(-c5ccc6ccc7ccccc7c6c5)cc4)cc3)c3ccccc3-c3ccc4c(c3)oc3c5ccccc5ccc43)cc2)cc(-n2c3ccccc3c3ccccc32)c1 ; c1cc(-c2ccc(N(c3ccc(-c4ccc(-c5ccc6c(ccc7ccccc76)c5)cc4)cc3)c3ccccc3-c3ccc4c(c3)oc3c5ccccc5ccc43)cc2)cc(-n2c3ccccc3c3ccccc32)c1]

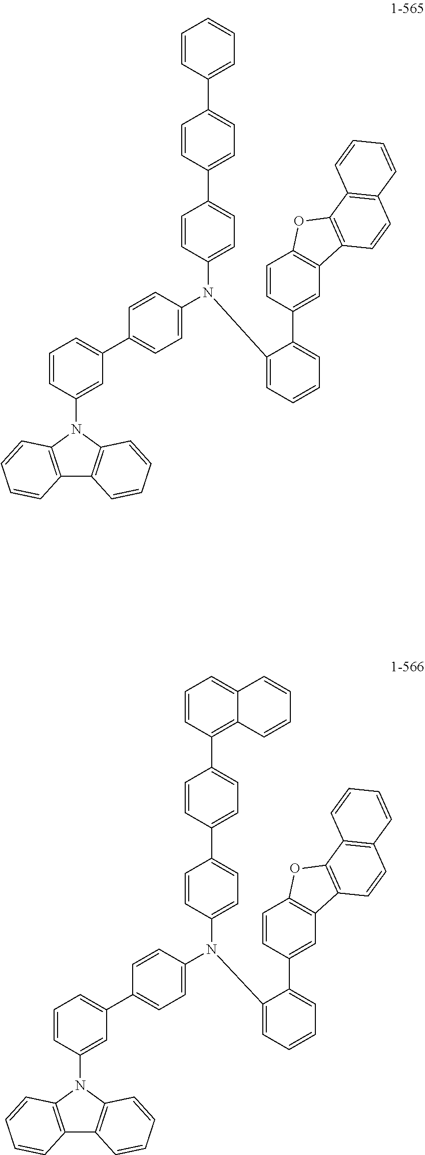 [SMILES: c1ccc(-c2ccc(-c3ccc(N(c4ccc(-c5cccc(-n6c7ccccc7c7ccccc76)c5)cc4)c4ccccc4-c4ccc5oc6c7ccccc7ccc6c5c4)cc3)cc2)cc1 ; c1cc(-c2ccc(N(c3ccc(-c4ccc(-c5cccc6ccccc56)cc4)cc3)c3ccccc3-c3ccc4oc5c6ccccc6ccc5c4c3)cc2)cc(-n2c3ccccc3c3ccccc32)c1]

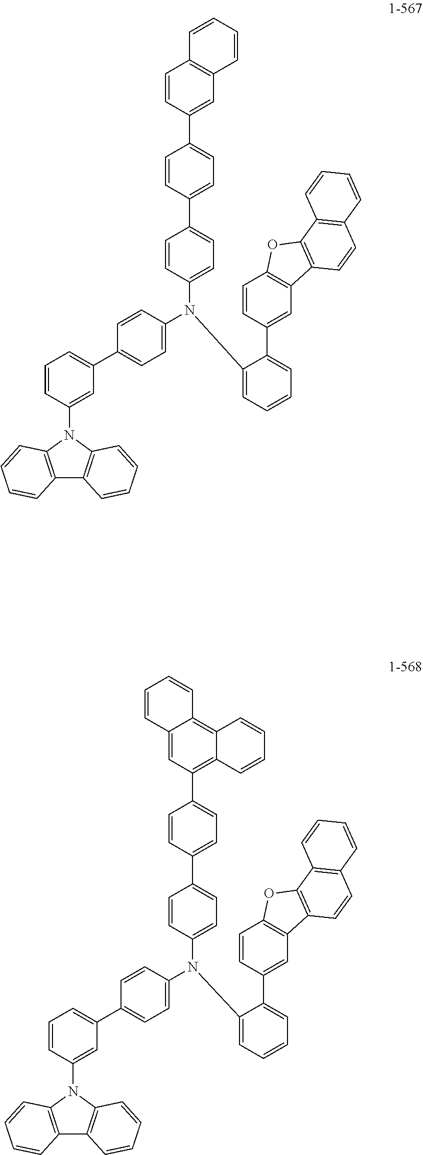 [SMILES: c1cc(-c2ccc(N(c3ccc(-c4ccc(-c5ccc6ccccc6c5)cc4)cc3)c3ccccc3-c3ccc4oc5c6ccccc6ccc5c4c3)cc2)cc(-n2c3ccccc3c3ccccc32)c1 ; c1cc(-c2ccc(N(c3ccc(-c4ccc(-c5cc6ccccc6c6ccccc56)cc4)cc3)c3ccccc3-c3ccc4oc5c6ccccc6ccc5c4c3)cc2)cc(-n2c3ccccc3c3ccccc32)c1]

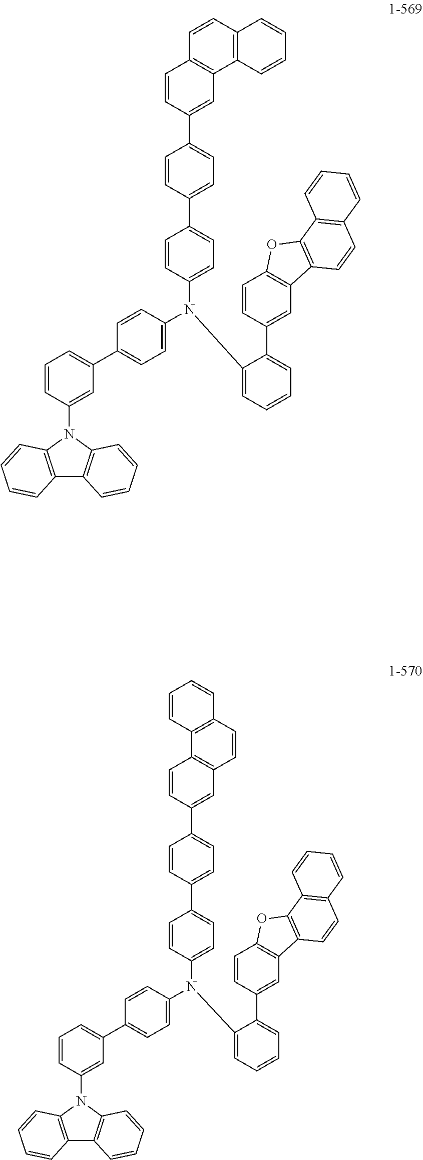 [SMILES: c1cc(-c2ccc(N(c3ccc(-c4ccc(-c5ccc6ccc7ccccc7c6c5)cc4)cc3)c3ccccc3-c3ccc4oc5c6ccccc6ccc5c4c3)cc2)cc(-n2c3ccccc3c3ccccc32)c1 ; c1cc(-c2ccc(N(c3ccc(-c4ccc(-c5ccc6c(ccc7ccccc76)c5)cc4)cc3)c3ccccc3-c3ccc4oc5c6ccccc6ccc5c4c3)cc2)cc(-n2c3ccccc3c3ccccc32)c1]

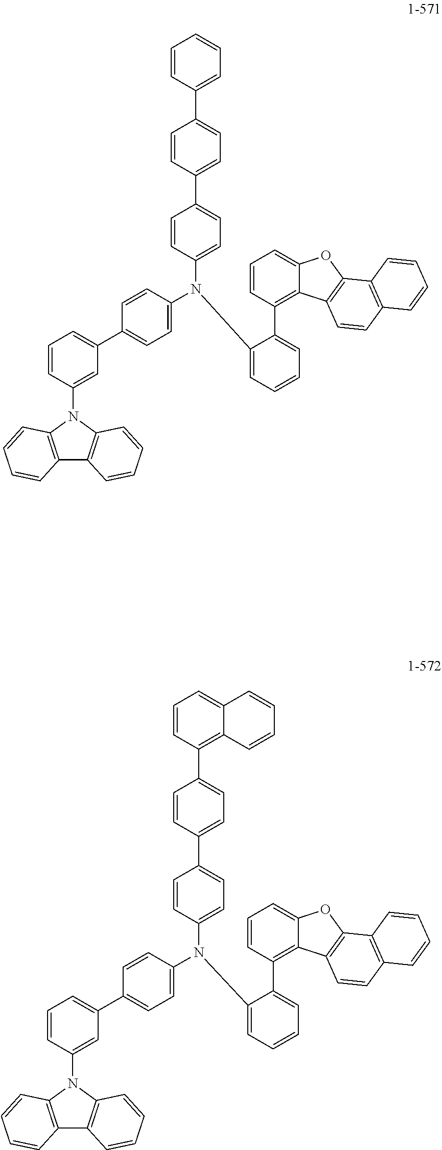 [SMILES: c1ccc(-c2ccc(-c3ccc(N(c4ccc(-c5cccc(-n6c7ccccc7c7ccccc76)c5)cc4)c4ccccc4-c4cccc5oc6c7ccccc7ccc6c45)cc3)cc2)cc1 ; c1cc(-c2ccc(N(c3ccc(-c4ccc(-c5cccc6ccccc56)cc4)cc3)c3ccccc3-c3cccc4oc5c6ccccc6ccc5c34)cc2)cc(-n2c3ccccc3c3ccccc32)c1]

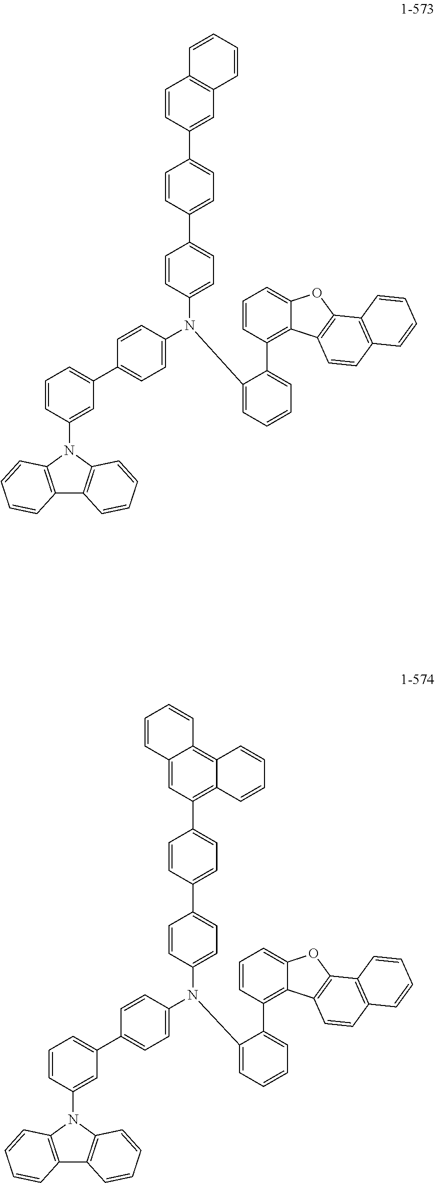 [SMILES: c1cc(-c2ccc(N(c3ccc(-c4ccc(-c5ccc6ccccc6c5)cc4)cc3)c3ccccc3-c3cccc4oc5c6ccccc6ccc5c34)cc2)cc(-n2c3ccccc3c3ccccc32)c1 ; c1cc(-c2ccc(N(c3ccc(-c4ccc(-c5cc6ccccc6c6ccccc56)cc4)cc3)c3ccccc3-c3cccc4oc5c6ccccc6ccc5c34)cc2)cc(-n2c3ccccc3c3ccccc32)c1]

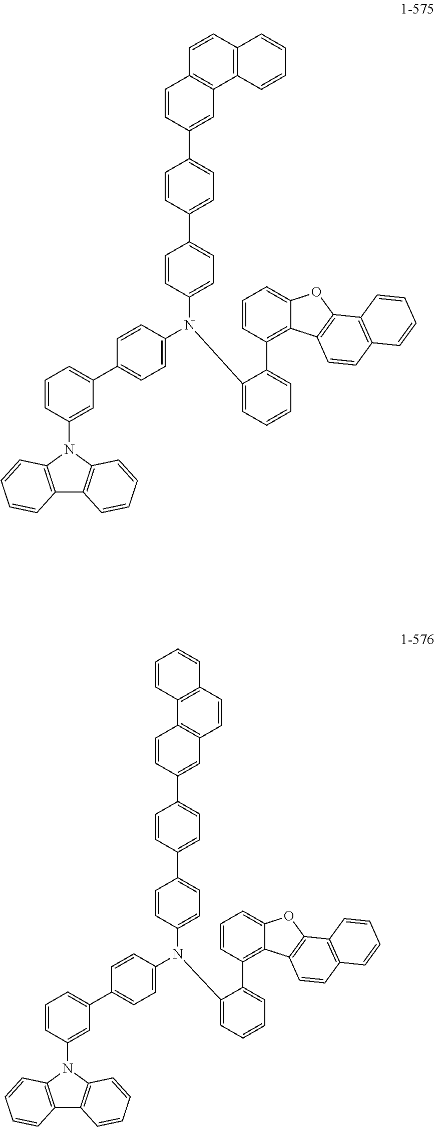 [SMILES: c1cc(-c2ccc(N(c3ccc(-c4ccc(-c5ccc6ccc7ccccc7c6c5)cc4)cc3)c3ccccc3-c3cccc4oc5c6ccccc6ccc5c34)cc2)cc(-n2c3ccccc3c3ccccc32)c1 ; c1cc(-c2ccc(N(c3ccc(-c4ccc(-c5ccc6c(ccc7ccccc76)c5)cc4)cc3)c3ccccc3-c3cccc4oc5c6ccccc6ccc5c34)cc2)cc(-n2c3ccccc3c3ccccc32)c1]

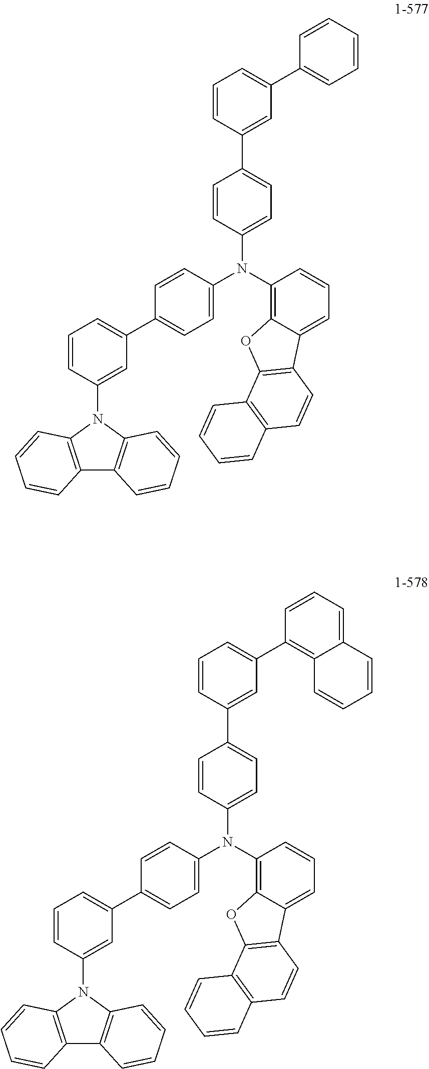 [SMILES: c1ccc(-c2cccc(-c3ccc(N(c4ccc(-c5cccc(-n6c7ccccc7c7ccccc76)c5)cc4)c4cccc5c4oc4c6ccccc6ccc54)cc3)c2)cc1 ; c1cc(-c2ccc(N(c3ccc(-c4cccc(-n5c6ccccc6c6ccccc65)c4)cc3)c3cccc4c3oc3c5ccccc5ccc43)cc2)cc(-c2cccc3ccccc23)c1]

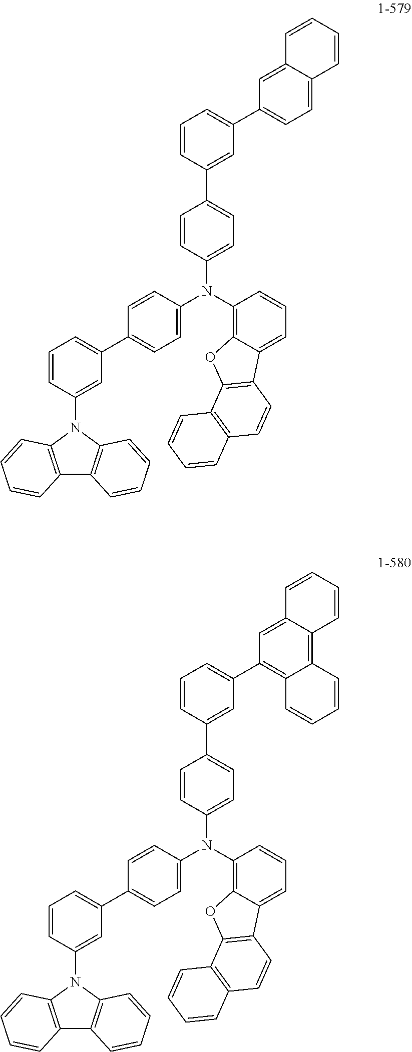 [SMILES: c1cc(-c2ccc(N(c3ccc(-c4cccc(-n5c6ccccc6c6ccccc65)c4)cc3)c3cccc4c3oc3c5ccccc5ccc43)cc2)cc(-c2ccc3ccccc3c2)c1 ; c1cc(-c2ccc(N(c3ccc(-c4cccc(-n5c6ccccc6c6ccccc65)c4)cc3)c3cccc4c3oc3c5ccccc5ccc43)cc2)cc(-c2cc3ccccc3c3ccccc23)c1]

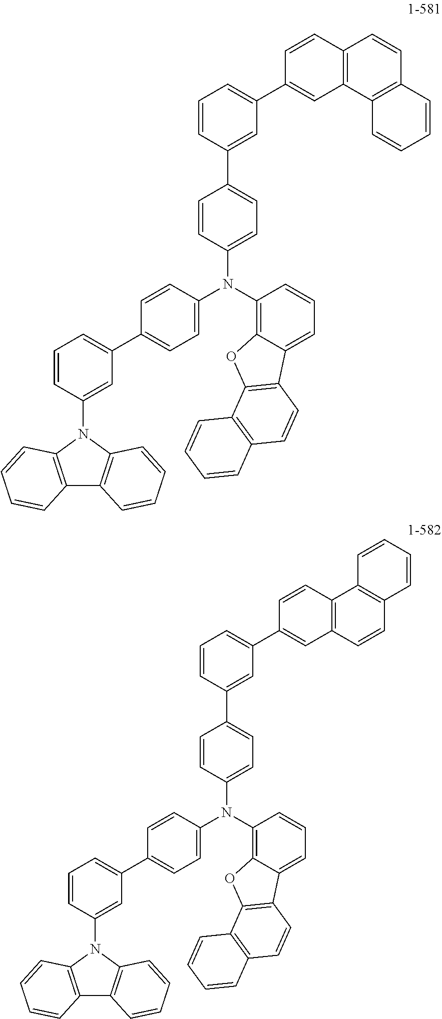 [SMILES: c1cc(-c2ccc(N(c3ccc(-c4cccc(-n5c6ccccc6c6ccccc65)c4)cc3)c3cccc4c3oc3c5ccccc5ccc43)cc2)cc(-c2ccc3ccc4ccccc4c3c2)c1 ; c1cc(-c2ccc(N(c3ccc(-c4cccc(-n5c6ccccc6c6ccccc65)c4)cc3)c3cccc4c3oc3c5ccccc5ccc43)cc2)cc(-c2ccc3c(ccc4ccccc43)c2)c1]

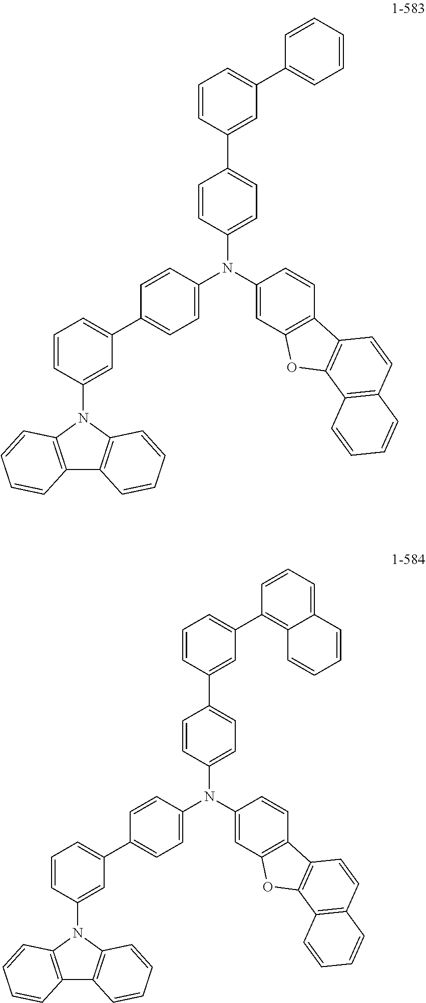 [SMILES: c1ccc(-c2cccc(-c3ccc(N(c4ccc(-c5cccc(-n6c7ccccc7c7ccccc76)c5)cc4)c4ccc5c(c4)oc4c6ccccc6ccc54)cc3)c2)cc1 ; c1cc(-c2ccc(N(c3ccc(-c4cccc(-n5c6ccccc6c6ccccc65)c4)cc3)c3ccc4c(c3)oc3c5ccccc5ccc43)cc2)cc(-c2cccc3ccccc23)c1]

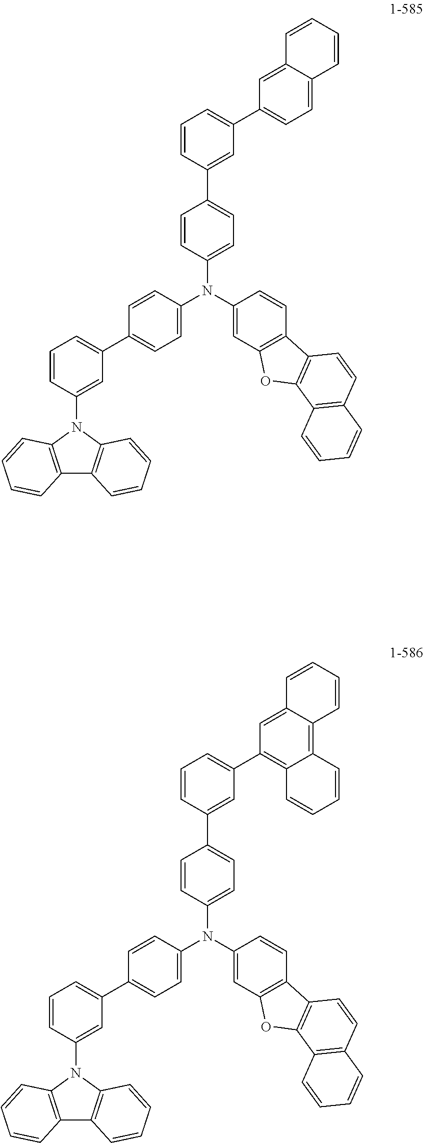 [SMILES: c1cc(-c2ccc(N(c3ccc(-c4cccc(-n5c6ccccc6c6ccccc65)c4)cc3)c3ccc4c(c3)oc3c5ccccc5ccc43)cc2)cc(-c2ccc3ccccc3c2)c1 ; c1cc(-c2ccc(N(c3ccc(-c4cccc(-n5c6ccccc6c6ccccc65)c4)cc3)c3ccc4c(c3)oc3c5ccccc5ccc43)cc2)cc(-c2cc3ccccc3c3ccccc23)c1]

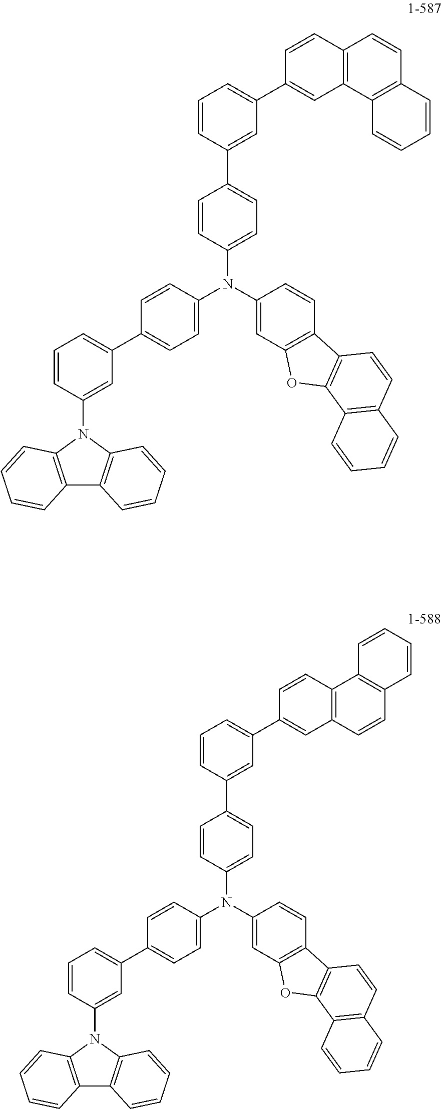 [SMILES: c1cc(-c2ccc(N(c3ccc(-c4cccc(-n5c6ccccc6c6ccccc65)c4)cc3)c3ccc4c(c3)oc3c5ccccc5ccc43)cc2)cc(-c2ccc3ccc4ccccc4c3c2)c1 ; c1cc(-c2ccc(N(c3ccc(-c4cccc(-n5c6ccccc6c6ccccc65)c4)cc3)c3ccc4c(c3)oc3c5ccccc5ccc43)cc2)cc(-c2ccc3c(ccc4ccccc43)c2)c1]

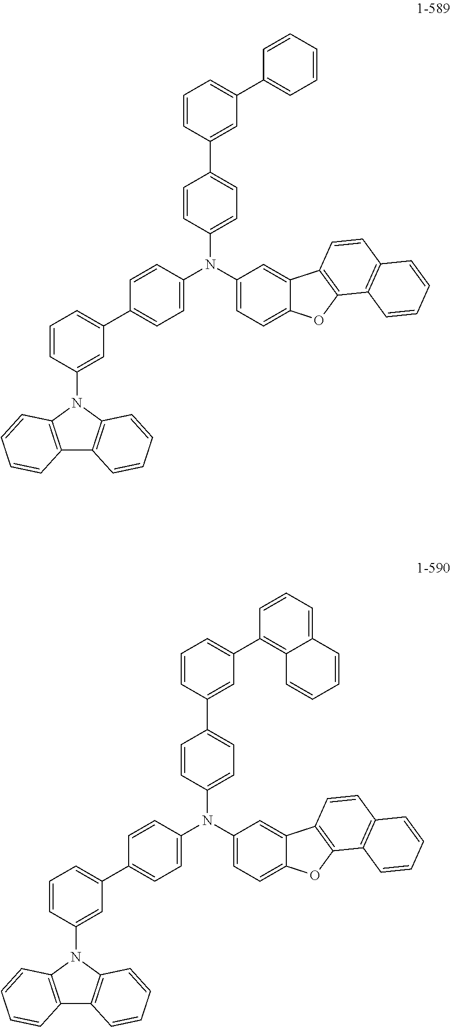 [SMILES: c1ccc(-c2cccc(-c3ccc(N(c4ccc(-c5cccc(-n6c7ccccc7c7ccccc76)c5)cc4)c4ccc5oc6c7ccccc7ccc6c5c4)cc3)c2)cc1 ; c1cc(-c2ccc(N(c3ccc(-c4cccc(-n5c6ccccc6c6ccccc65)c4)cc3)c3ccc4oc5c6ccccc6ccc5c4c3)cc2)cc(-c2cccc3ccccc23)c1]

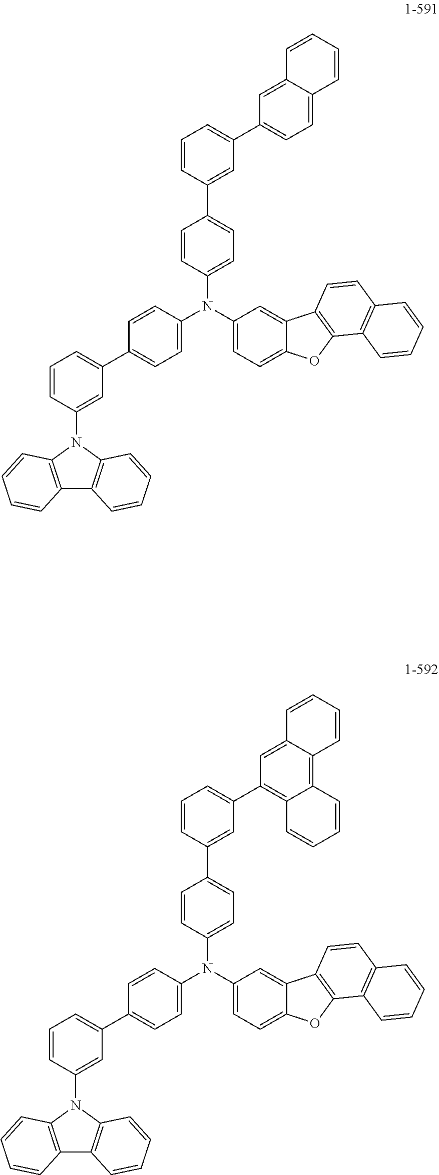 [SMILES: c1cc(-c2ccc(N(c3ccc(-c4cccc(-n5c6ccccc6c6ccccc65)c4)cc3)c3ccc4oc5c6ccccc6ccc5c4c3)cc2)cc(-c2ccc3ccccc3c2)c1 ; c1cc(-c2ccc(N(c3ccc(-c4cccc(-n5c6ccccc6c6ccccc65)c4)cc3)c3ccc4oc5c6ccccc6ccc5c4c3)cc2)cc(-c2cc3ccccc3c3ccccc23)c1]

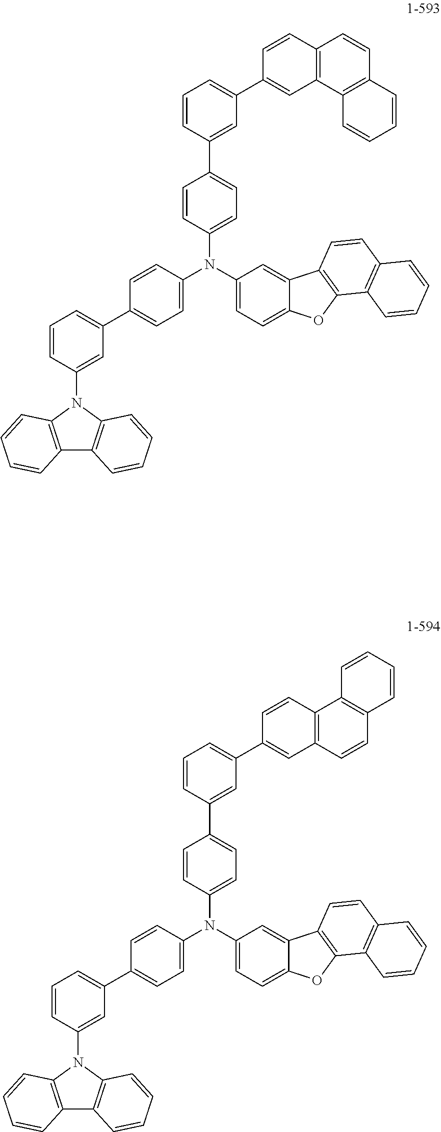 [SMILES: c1cc(-c2ccc(N(c3ccc(-c4cccc(-n5c6ccccc6c6ccccc65)c4)cc3)c3ccc4oc5c6ccccc6ccc5c4c3)cc2)cc(-c2ccc3ccc4ccccc4c3c2)c1 ; c1cc(-c2ccc(N(c3ccc(-c4cccc(-n5c6ccccc6c6ccccc65)c4)cc3)c3ccc4oc5c6ccccc6ccc5c4c3)cc2)cc(-c2ccc3c(ccc4ccccc43)c2)c1]

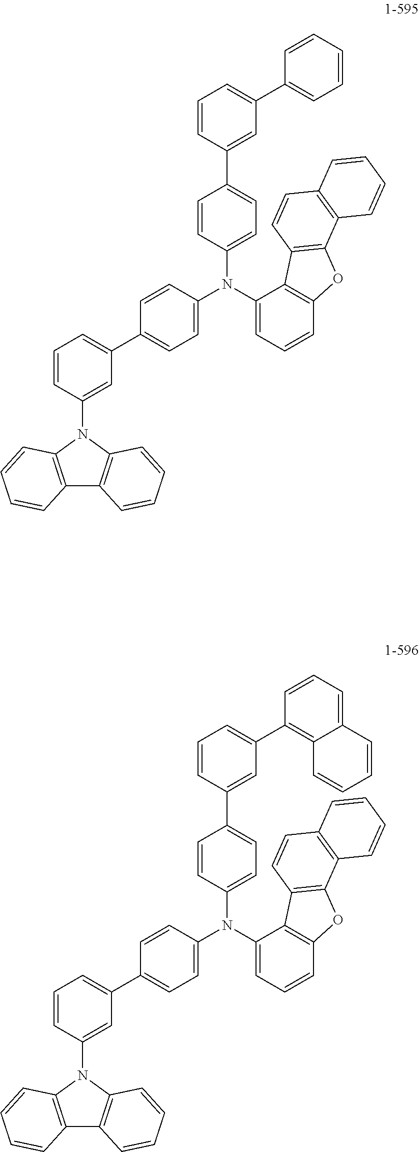 [SMILES: c1ccc(-c2cccc(-c3ccc(N(c4ccc(-c5cccc(-n6c7ccccc7c7ccccc76)c5)cc4)c4cccc5oc6c7ccccc7ccc6c45)cc3)c2)cc1 ; c1cc(-c2ccc(N(c3ccc(-c4cccc(-n5c6ccccc6c6ccccc65)c4)cc3)c3cccc4oc5c6ccccc6ccc5c34)cc2)cc(-c2cccc3ccccc23)c1]

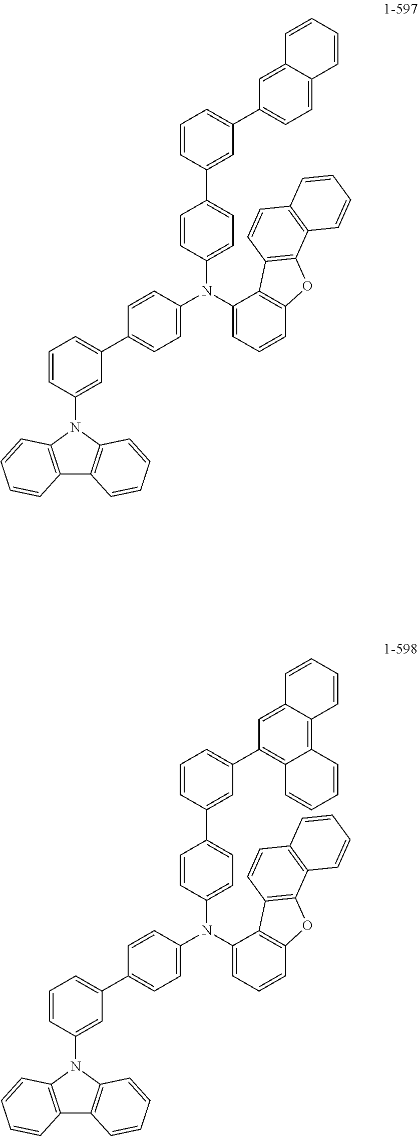 [SMILES: c1cc(-c2ccc(N(c3ccc(-c4cccc(-n5c6ccccc6c6ccccc65)c4)cc3)c3cccc4oc5c6ccccc6ccc5c34)cc2)cc(-c2ccc3ccccc3c2)c1 ; c1cc(-c2ccc(N(c3ccc(-c4cccc(-n5c6ccccc6c6ccccc65)c4)cc3)c3cccc4oc5c6ccccc6ccc5c34)cc2)cc(-c2cc3ccccc3c3ccccc23)c1]

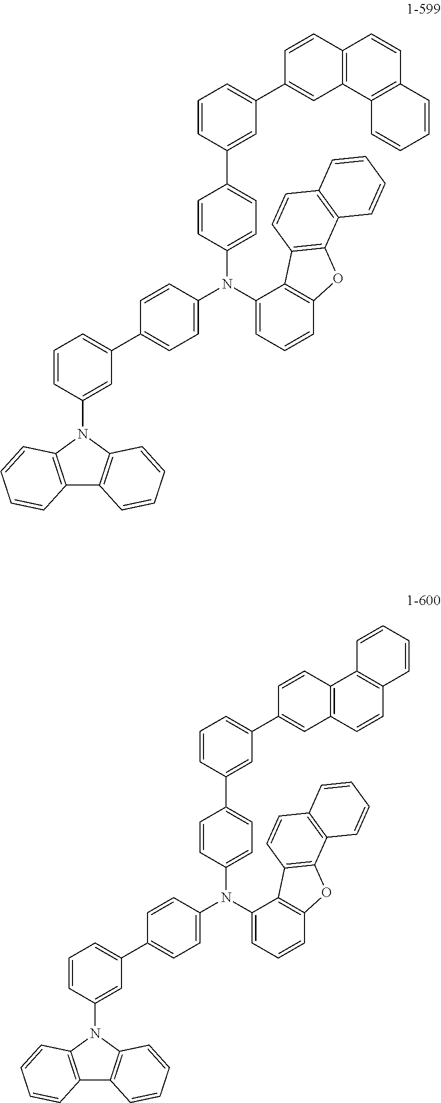 [SMILES: c1cc(-c2ccc(N(c3ccc(-c4cccc(-n5c6ccccc6c6ccccc65)c4)cc3)c3cccc4oc5c6ccccc6ccc5c34)cc2)cc(-c2ccc3ccc4ccccc4c3c2)c1 ; c1cc(-c2ccc(N(c3ccc(-c4cccc(-n5c6ccccc6c6ccccc65)c4)cc3)c3cccc4oc5c6ccccc6ccc5c34)cc2)cc(-c2ccc3c(ccc4ccccc43)c2)c1]

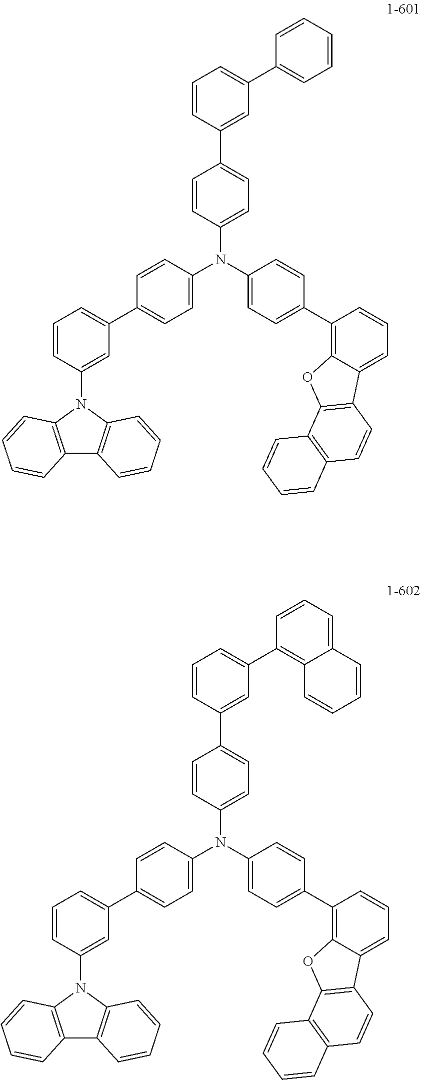 [SMILES: c1ccc(-c2cccc(-c3ccc(N(c4ccc(-c5cccc(-n6c7ccccc7c7ccccc76)c5)cc4)c4ccc(-c5cccc6c5oc5c7ccccc7ccc65)cc4)cc3)c2)cc1 ; c1cc(-c2ccc(N(c3ccc(-c4cccc(-n5c6ccccc6c6ccccc65)c4)cc3)c3ccc(-c4cccc5c4oc4c6ccccc6ccc54)cc3)cc2)cc(-c2cccc3ccccc23)c1]

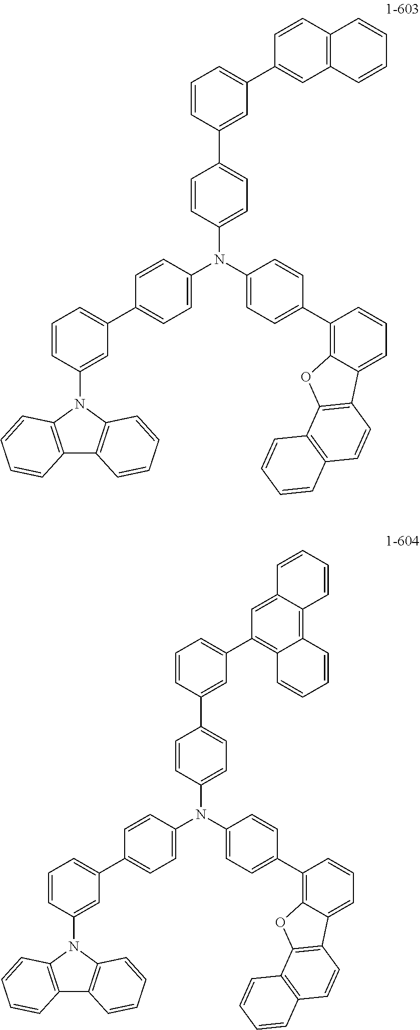 [SMILES: c1cc(-c2ccc(N(c3ccc(-c4cccc(-n5c6ccccc6c6ccccc65)c4)cc3)c3ccc(-c4cccc5c4oc4c6ccccc6ccc54)cc3)cc2)cc(-c2ccc3ccccc3c2)c1 ; c1cc(-c2ccc(N(c3ccc(-c4cccc(-n5c6ccccc6c6ccccc65)c4)cc3)c3ccc(-c4cccc5c4oc4c6ccccc6ccc54)cc3)cc2)cc(-c2cc3ccccc3c3ccccc23)c1]

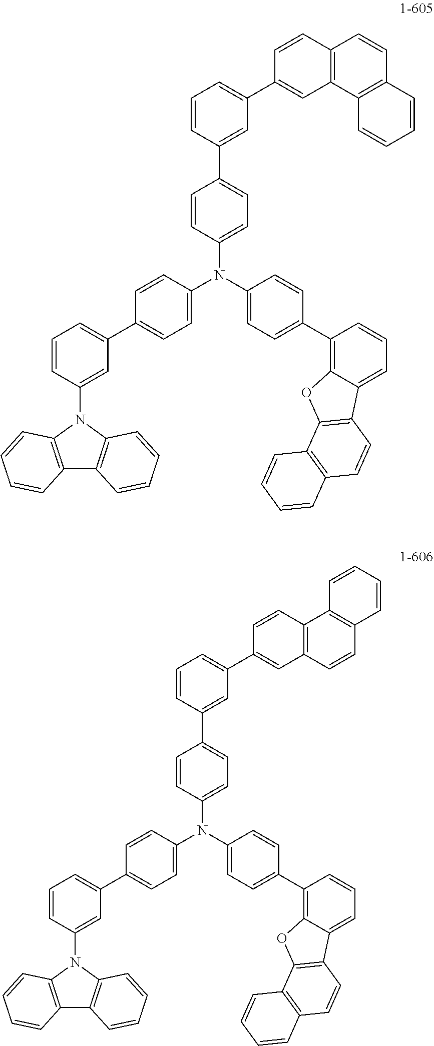 [SMILES: c1cc(-c2ccc(N(c3ccc(-c4cccc(-n5c6ccccc6c6ccccc65)c4)cc3)c3ccc(-c4cccc5c4oc4c6ccccc6ccc54)cc3)cc2)cc(-c2ccc3ccc4ccccc4c3c2)c1 ; c1cc(-c2ccc(N(c3ccc(-c4cccc(-n5c6ccccc6c6ccccc65)c4)cc3)c3ccc(-c4cccc5c4oc4c6ccccc6ccc54)cc3)cc2)cc(-c2ccc3c(ccc4ccccc43)c2)c1]

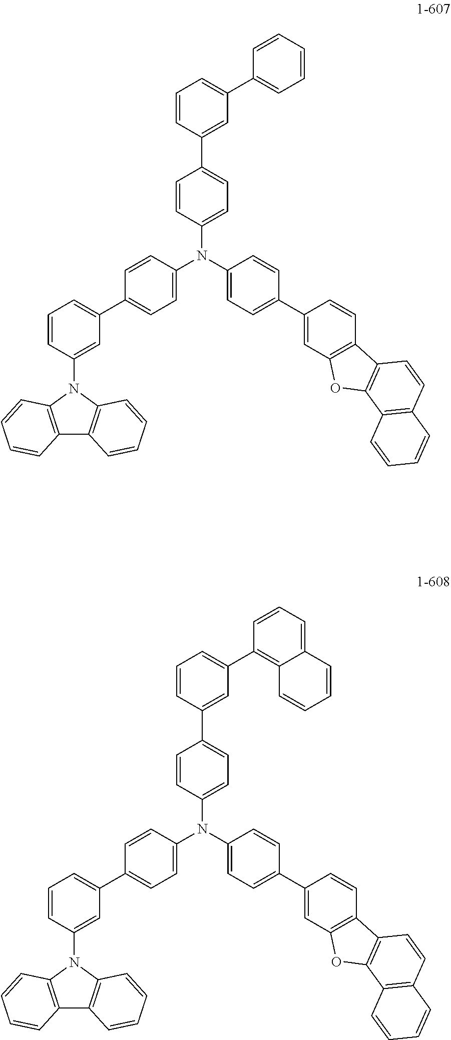 [SMILES: c1ccc(-c2cccc(-c3ccc(N(c4ccc(-c5cccc(-n6c7ccccc7c7ccccc76)c5)cc4)c4ccc(-c5ccc6c(c5)oc5c7ccccc7ccc65)cc4)cc3)c2)cc1 ; c1cc(-c2ccc(N(c3ccc(-c4cccc(-n5c6ccccc6c6ccccc65)c4)cc3)c3ccc(-c4ccc5c(c4)oc4c6ccccc6ccc54)cc3)cc2)cc(-c2cccc3ccccc23)c1]

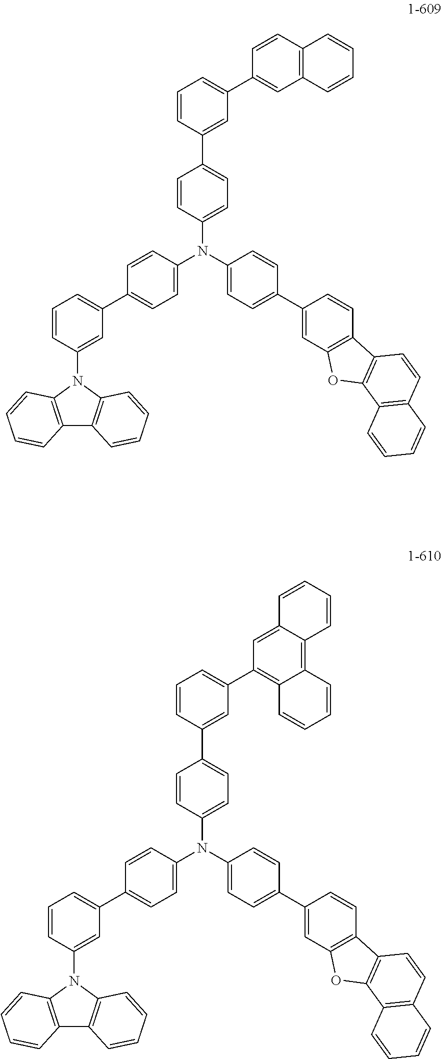 [SMILES: c1cc(-c2ccc(N(c3ccc(-c4cccc(-n5c6ccccc6c6ccccc65)c4)cc3)c3ccc(-c4ccc5c(c4)oc4c6ccccc6ccc54)cc3)cc2)cc(-c2ccc3ccccc3c2)c1 ; c1cc(-c2ccc(N(c3ccc(-c4cccc(-n5c6ccccc6c6ccccc65)c4)cc3)c3ccc(-c4ccc5c(c4)oc4c6ccccc6ccc54)cc3)cc2)cc(-c2cc3ccccc3c3ccccc23)c1]

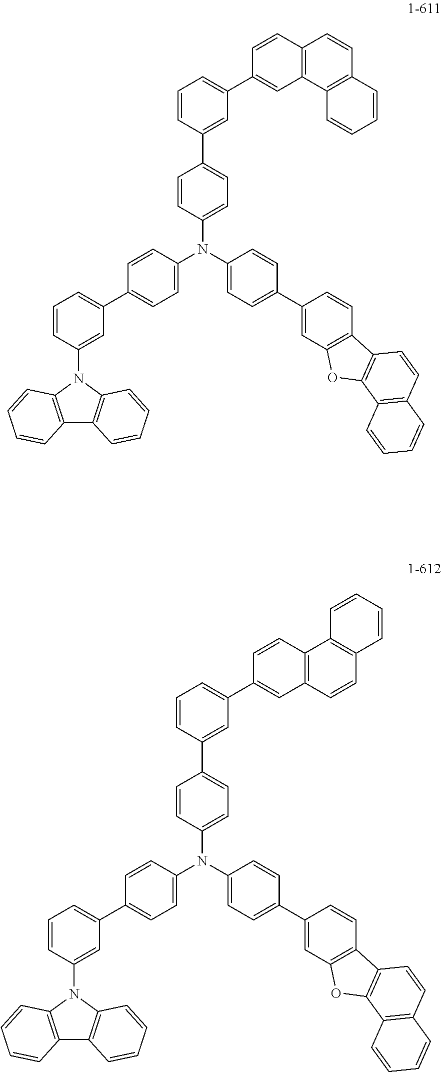 [SMILES: c1cc(-c2ccc(N(c3ccc(-c4cccc(-n5c6ccccc6c6ccccc65)c4)cc3)c3ccc(-c4ccc5c(c4)oc4c6ccccc6ccc54)cc3)cc2)cc(-c2ccc3ccc4ccccc4c3c2)c1 ; c1cc(-c2ccc(N(c3ccc(-c4cccc(-n5c6ccccc6c6ccccc65)c4)cc3)c3ccc(-c4ccc5c(c4)oc4c6ccccc6ccc54)cc3)cc2)cc(-c2ccc3c(ccc4ccccc43)c2)c1]

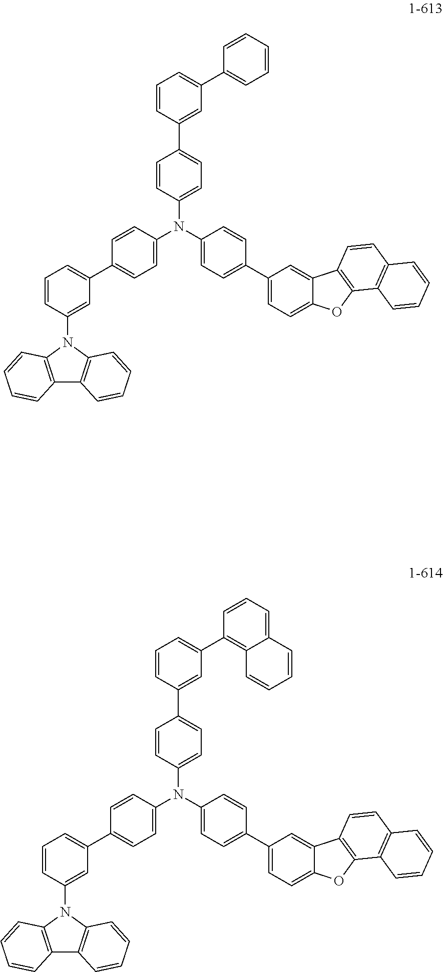 [SMILES: c1ccc(-c2cccc(-c3ccc(N(c4ccc(-c5cccc(-n6c7ccccc7c7ccccc76)c5)cc4)c4ccc(-c5ccc6oc7c8ccccc8ccc7c6c5)cc4)cc3)c2)cc1 ; c1cc(-c2ccc(N(c3ccc(-c4cccc(-n5c6ccccc6c6ccccc65)c4)cc3)c3ccc(-c4ccc5oc6c7ccccc7ccc6c5c4)cc3)cc2)cc(-c2cccc3ccccc23)c1]

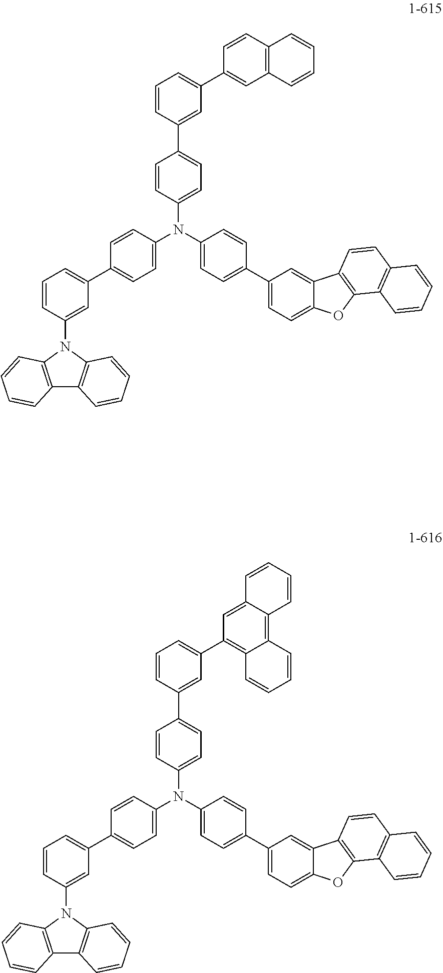 [SMILES: c1cc(-c2ccc(N(c3ccc(-c4cccc(-n5c6ccccc6c6ccccc65)c4)cc3)c3ccc(-c4ccc5oc6c7ccccc7ccc6c5c4)cc3)cc2)cc(-c2ccc3ccccc3c2)c1 ; c1cc(-c2ccc(N(c3ccc(-c4cccc(-n5c6ccccc6c6ccccc65)c4)cc3)c3ccc(-c4ccc5oc6c7ccccc7ccc6c5c4)cc3)cc2)cc(-c2cc3ccccc3c3ccccc23)c1]

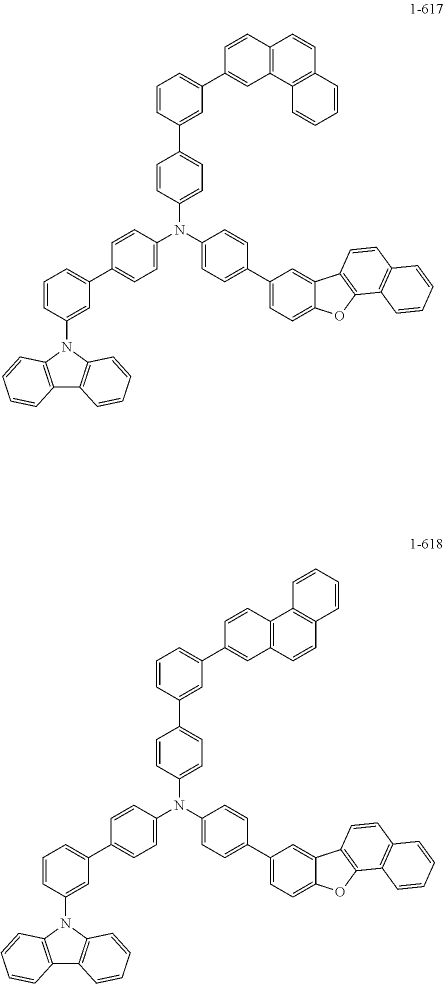 [SMILES: c1cc(-c2ccc(N(c3ccc(-c4cccc(-n5c6ccccc6c6ccccc65)c4)cc3)c3ccc(-c4ccc5oc6c7ccccc7ccc6c5c4)cc3)cc2)cc(-c2ccc3ccc4ccccc4c3c2)c1 ; c1cc(-c2ccc(N(c3ccc(-c4cccc(-n5c6ccccc6c6ccccc65)c4)cc3)c3ccc(-c4ccc5oc6c7ccccc7ccc6c5c4)cc3)cc2)cc(-c2ccc3c(ccc4ccccc43)c2)c1]

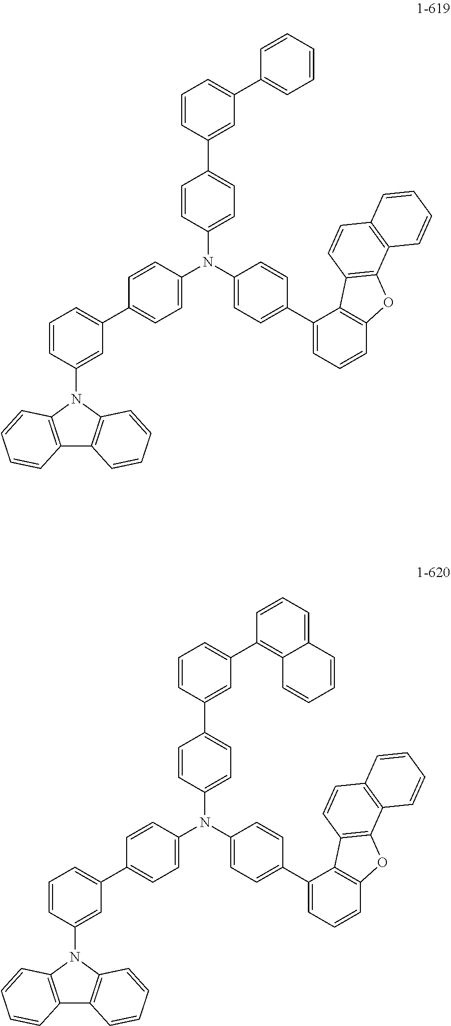 [SMILES: c1ccc(-c2cccc(-c3ccc(N(c4ccc(-c5cccc(-n6c7ccccc7c7ccccc76)c5)cc4)c4ccc(-c5cccc6oc7c8ccccc8ccc7c56)cc4)cc3)c2)cc1 ; c1cc(-c2ccc(N(c3ccc(-c4cccc(-n5c6ccccc6c6ccccc65)c4)cc3)c3ccc(-c4cccc5oc6c7ccccc7ccc6c45)cc3)cc2)cc(-c2cccc3ccccc23)c1]

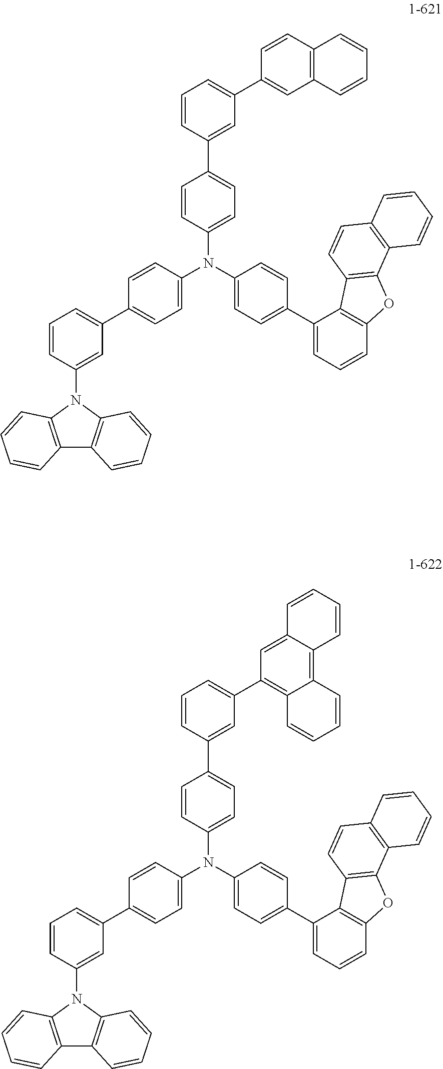 [SMILES: c1cc(-c2ccc(N(c3ccc(-c4cccc(-n5c6ccccc6c6ccccc65)c4)cc3)c3ccc(-c4cccc5oc6c7ccccc7ccc6c45)cc3)cc2)cc(-c2ccc3ccccc3c2)c1 ; c1cc(-c2ccc(N(c3ccc(-c4cccc(-n5c6ccccc6c6ccccc65)c4)cc3)c3ccc(-c4cccc5oc6c7ccccc7ccc6c45)cc3)cc2)cc(-c2cc3ccccc3c3ccccc23)c1]

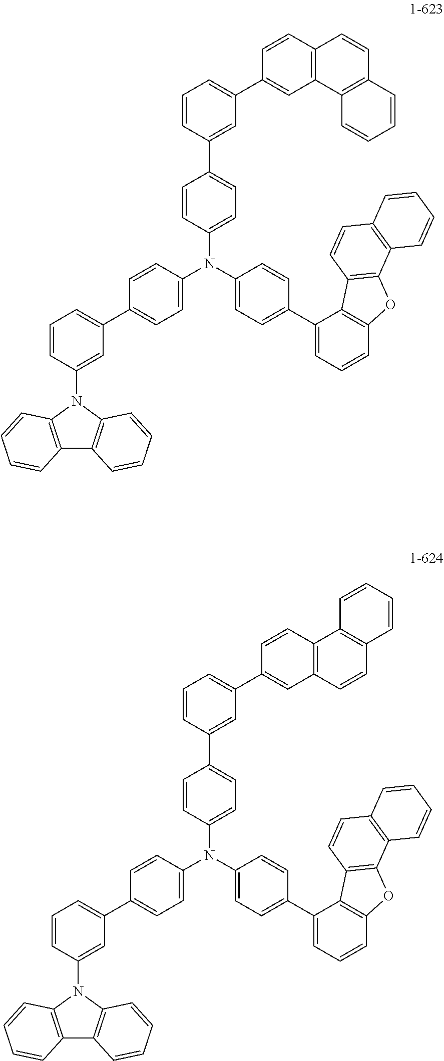 [SMILES: c1cc(-c2ccc(N(c3ccc(-c4cccc(-n5c6ccccc6c6ccccc65)c4)cc3)c3ccc(-c4cccc5oc6c7ccccc7ccc6c45)cc3)cc2)cc(-c2ccc3ccc4ccccc4c3c2)c1 ; c1cc(-c2ccc(N(c3ccc(-c4cccc(-n5c6ccccc6c6ccccc65)c4)cc3)c3ccc(-c4cccc5oc6c7ccccc7ccc6c45)cc3)cc2)cc(-c2ccc3c(ccc4ccccc43)c2)c1]

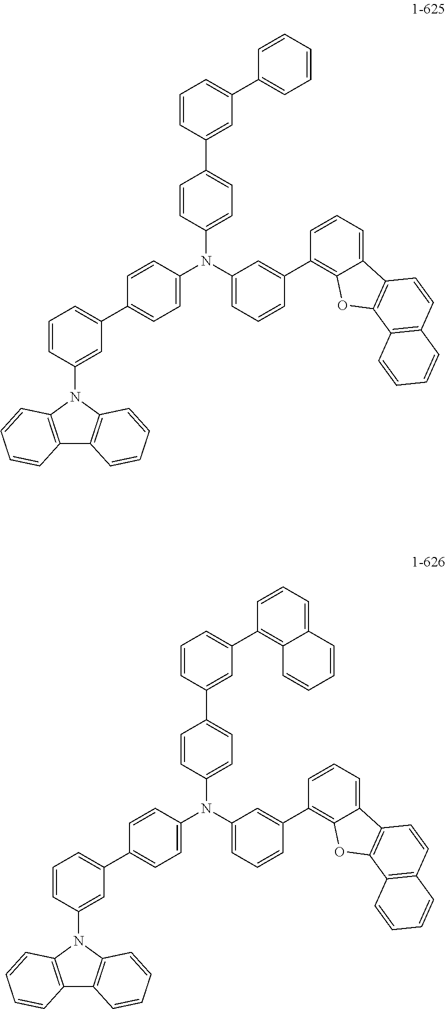 [SMILES: c1ccc(-c2cccc(-c3ccc(N(c4ccc(-c5cccc(-n6c7ccccc7c7ccccc76)c5)cc4)c4cccc(-c5cccc6c5oc5c7ccccc7ccc65)c4)cc3)c2)cc1 ; c1cc(-c2ccc(N(c3ccc(-c4cccc(-n5c6ccccc6c6ccccc65)c4)cc3)c3cccc(-c4cccc5c4oc4c6ccccc6ccc54)c3)cc2)cc(-c2cccc3ccccc23)c1]

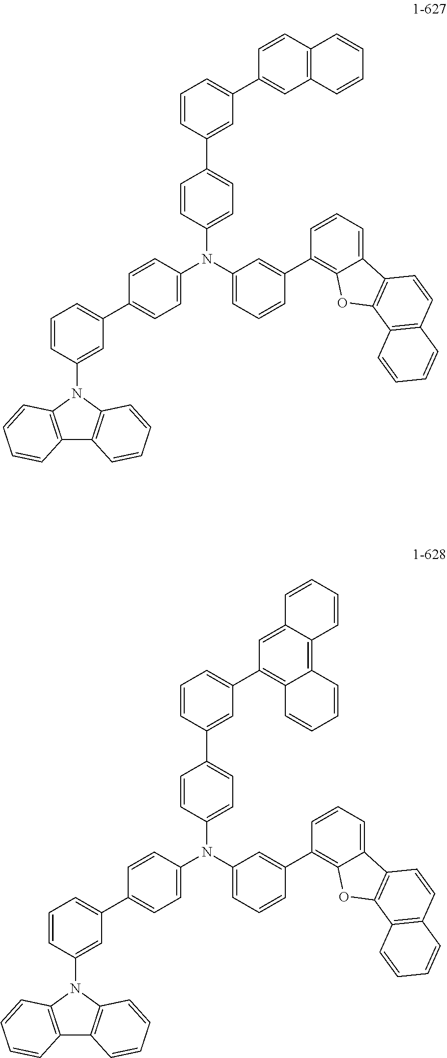 [SMILES: c1cc(-c2ccc(N(c3ccc(-c4cccc(-n5c6ccccc6c6ccccc65)c4)cc3)c3cccc(-c4cccc5c4oc4c6ccccc6ccc54)c3)cc2)cc(-c2ccc3ccccc3c2)c1 ; c1cc(-c2ccc(N(c3ccc(-c4cccc(-n5c6ccccc6c6ccccc65)c4)cc3)c3cccc(-c4cccc5c4oc4c6ccccc6ccc54)c3)cc2)cc(-c2cc3ccccc3c3ccccc23)c1]

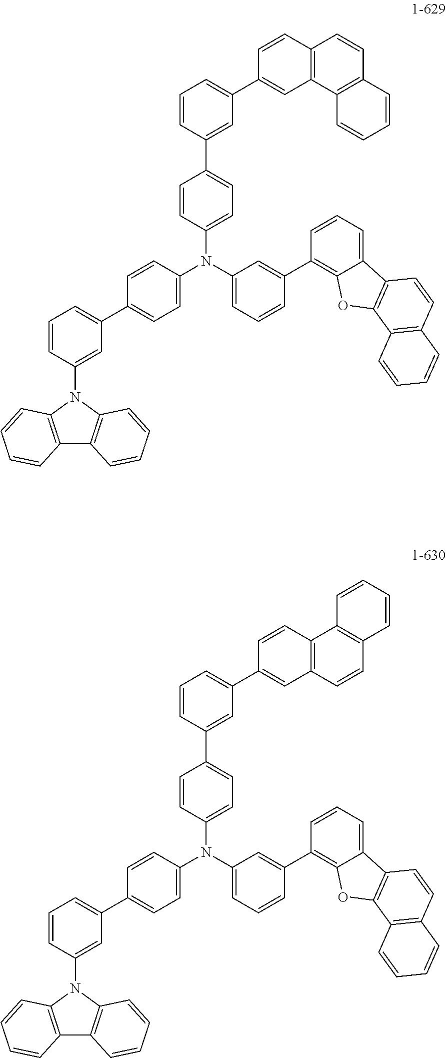 [SMILES: c1cc(-c2ccc(N(c3ccc(-c4cccc(-n5c6ccccc6c6ccccc65)c4)cc3)c3cccc(-c4cccc5c4oc4c6ccccc6ccc54)c3)cc2)cc(-c2ccc3ccc4ccccc4c3c2)c1 ; c1cc(-c2ccc(N(c3ccc(-c4cccc(-n5c6ccccc6c6ccccc65)c4)cc3)c3cccc(-c4cccc5c4oc4c6ccccc6ccc54)c3)cc2)cc(-c2ccc3c(ccc4ccccc43)c2)c1]

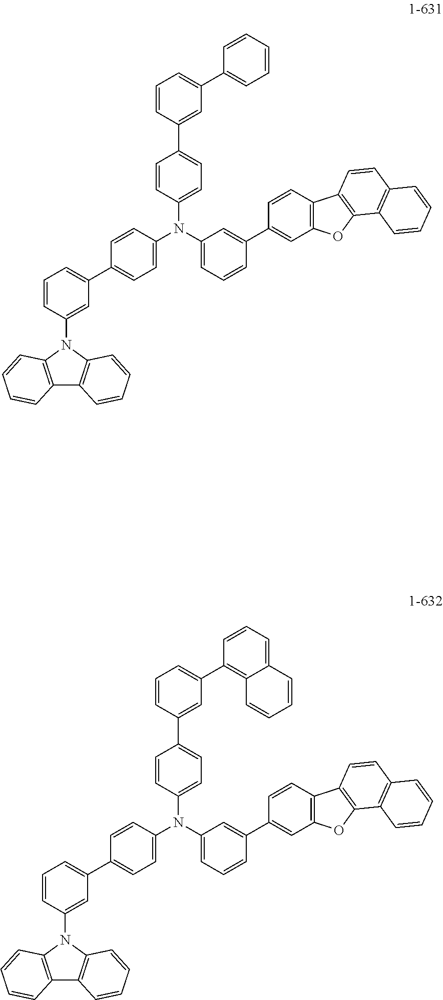 [SMILES: c1ccc(-c2cccc(-c3ccc(N(c4ccc(-c5cccc(-n6c7ccccc7c7ccccc76)c5)cc4)c4cccc(-c5ccc6c(c5)oc5c7ccccc7ccc65)c4)cc3)c2)cc1 ; c1cc(-c2ccc(N(c3ccc(-c4cccc(-n5c6ccccc6c6ccccc65)c4)cc3)c3cccc(-c4ccc5c(c4)oc4c6ccccc6ccc54)c3)cc2)cc(-c2cccc3ccccc23)c1]

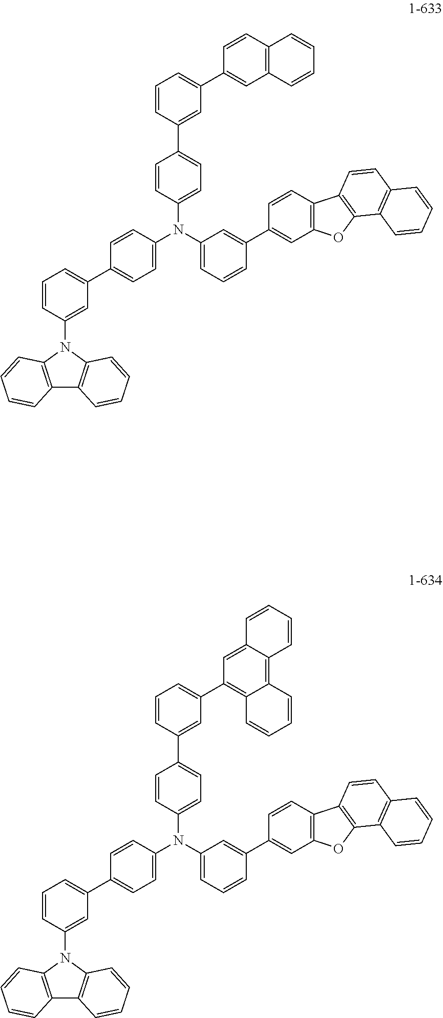 [SMILES: c1cc(-c2ccc(N(c3ccc(-c4cccc(-n5c6ccccc6c6ccccc65)c4)cc3)c3cccc(-c4ccc5c(c4)oc4c6ccccc6ccc54)c3)cc2)cc(-c2ccc3ccccc3c2)c1 ; c1cc(-c2ccc(N(c3ccc(-c4cccc(-n5c6ccccc6c6ccccc65)c4)cc3)c3cccc(-c4ccc5c(c4)oc4c6ccccc6ccc54)c3)cc2)cc(-c2cc3ccccc3c3ccccc23)c1]

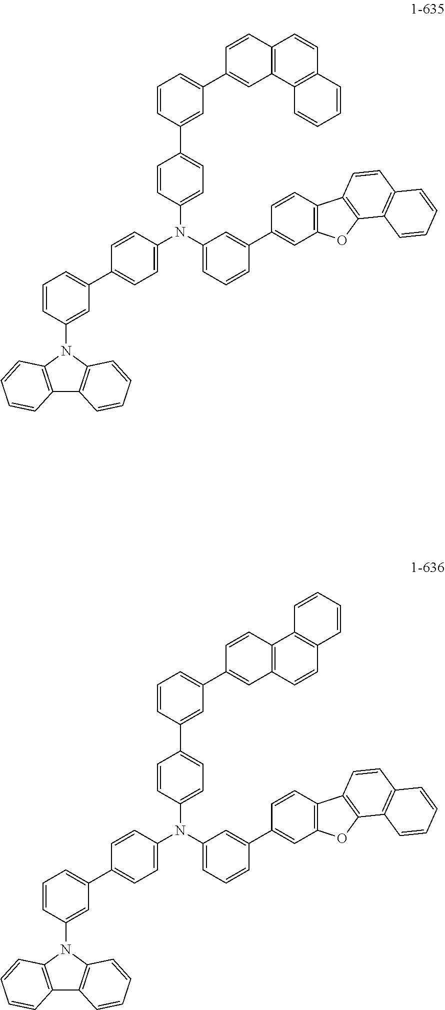 [SMILES: c1cc(-c2ccc(N(c3ccc(-c4cccc(-n5c6ccccc6c6ccccc65)c4)cc3)c3cccc(-c4ccc5c(c4)oc4c6ccccc6ccc54)c3)cc2)cc(-c2ccc3ccc4ccccc4c3c2)c1 ; c1cc(-c2ccc(N(c3ccc(-c4cccc(-n5c6ccccc6c6ccccc65)c4)cc3)c3cccc(-c4ccc5c(c4)oc4c6ccccc6ccc54)c3)cc2)cc(-c2ccc3c(ccc4ccccc43)c2)c1]

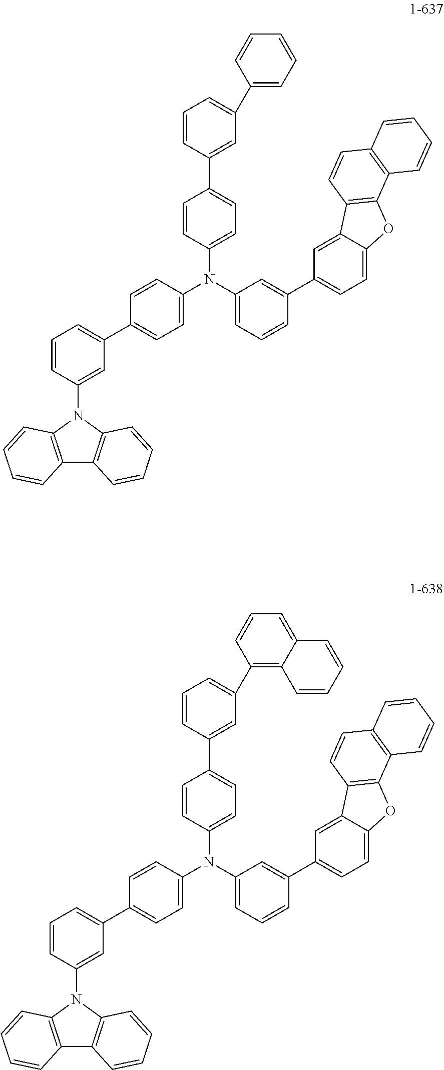 [SMILES: c1ccc(-c2cccc(-c3ccc(N(c4ccc(-c5cccc(-n6c7ccccc7c7ccccc76)c5)cc4)c4cccc(-c5ccc6oc7c8ccccc8ccc7c6c5)c4)cc3)c2)cc1 ; c1cc(-c2ccc(N(c3ccc(-c4cccc(-n5c6ccccc6c6ccccc65)c4)cc3)c3cccc(-c4ccc5oc6c7ccccc7ccc6c5c4)c3)cc2)cc(-c2cccc3ccccc23)c1]

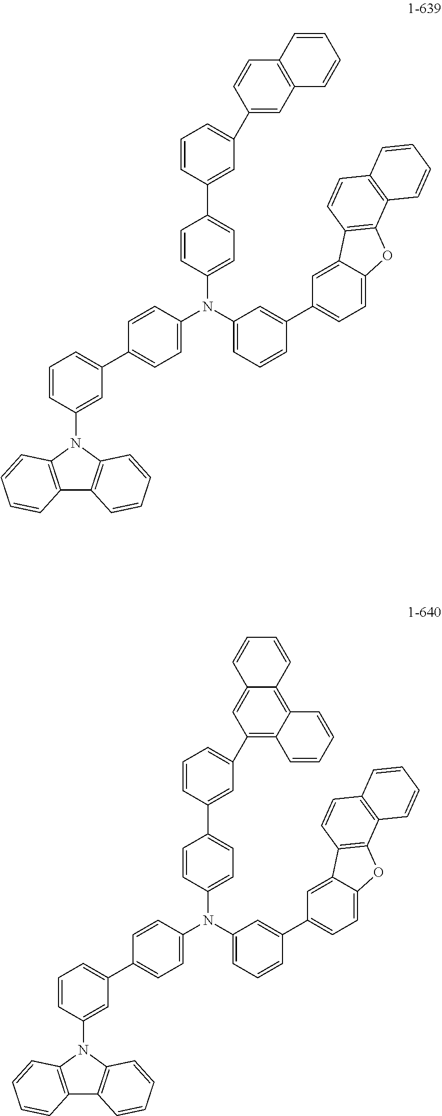 [SMILES: c1cc(-c2ccc(N(c3ccc(-c4cccc(-n5c6ccccc6c6ccccc65)c4)cc3)c3cccc(-c4ccc5oc6c7ccccc7ccc6c5c4)c3)cc2)cc(-c2ccc3ccccc3c2)c1 ; c1cc(-c2ccc(N(c3ccc(-c4cccc(-n5c6ccccc6c6ccccc65)c4)cc3)c3cccc(-c4ccc5oc6c7ccccc7ccc6c5c4)c3)cc2)cc(-c2cc3ccccc3c3ccccc23)c1]

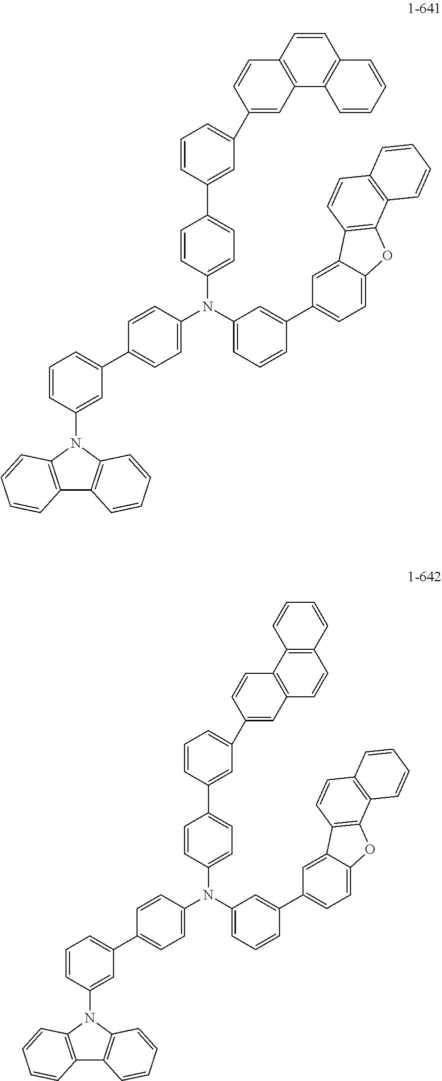 [SMILES: c1cc(-c2ccc(N(c3ccc(-c4cccc(-n5c6ccccc6c6ccccc65)c4)cc3)c3cccc(-c4ccc5oc6c7ccccc7ccc6c5c4)c3)cc2)cc(-c2ccc3ccc4ccccc4c3c2)c1 ; c1cc(-c2ccc(N(c3ccc(-c4cccc(-n5c6ccccc6c6ccccc65)c4)cc3)c3cccc(-c4ccc5oc6c7ccccc7ccc6c5c4)c3)cc2)cc(-c2ccc3c(ccc4ccccc43)c2)c1]

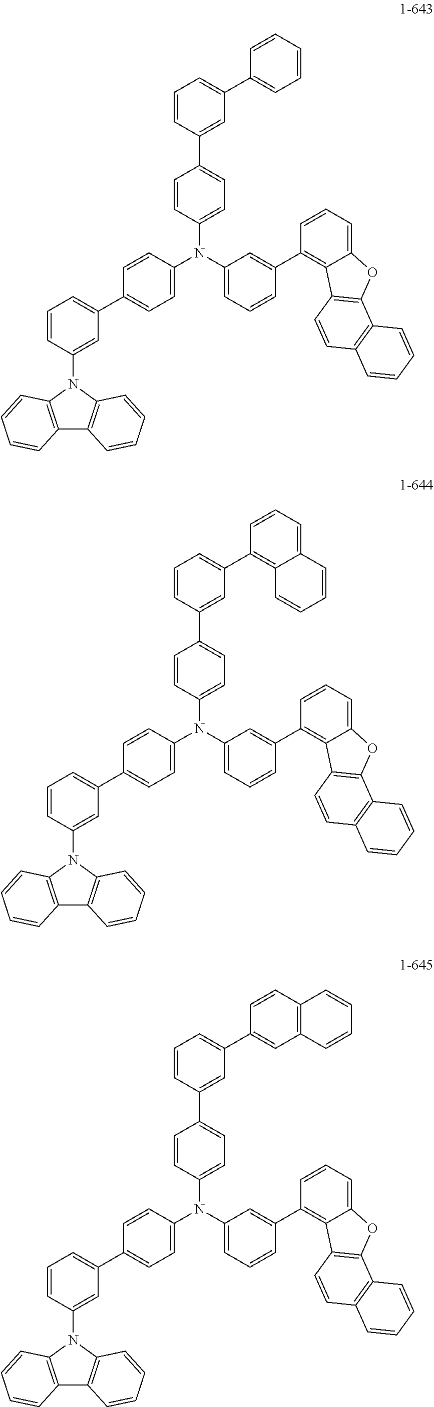 [SMILES: c1ccc(-c2cccc(-c3ccc(N(c4ccc(-c5cccc(-n6c7ccccc7c7ccccc76)c5)cc4)c4cccc(-c5cccc6oc7c8ccccc8ccc7c56)c4)cc3)c2)cc1 ; c1cc(-c2ccc(N(c3ccc(-c4cccc(-n5c6ccccc6c6ccccc65)c4)cc3)c3cccc(-c4cccc5oc6c7ccccc7ccc6c45)c3)cc2)cc(-c2cccc3ccccc23)c1 ; c1cc(-c2ccc(N(c3ccc(-c4cccc(-n5c6ccccc6c6ccccc65)c4)cc3)c3cccc(-c4cccc5oc6c7ccccc7ccc6c45)c3)cc2)cc(-c2ccc3ccccc3c2)c1]

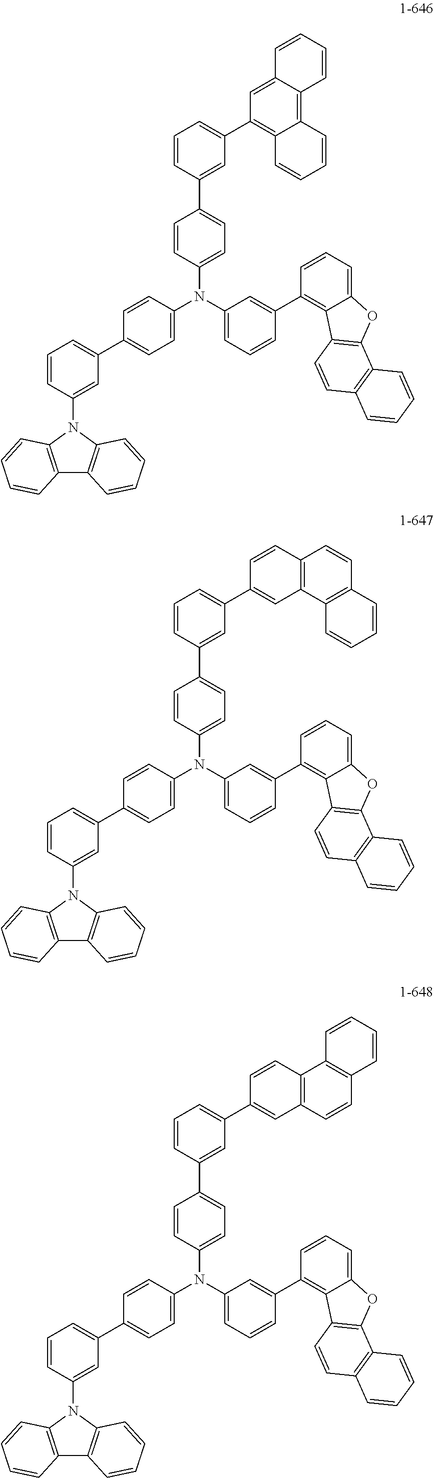 [SMILES: c1cc(-c2ccc(N(c3ccc(-c4cccc(-n5c6ccccc6c6ccccc65)c4)cc3)c3cccc(-c4cccc5oc6c7ccccc7ccc6c45)c3)cc2)cc(-c2cc3ccccc3c3ccccc23)c1 ; c1cc(-c2ccc(N(c3ccc(-c4cccc(-n5c6ccccc6c6ccccc65)c4)cc3)c3cccc(-c4cccc5oc6c7ccccc7ccc6c45)c3)cc2)cc(-c2ccc3ccc4ccccc4c3c2)c1 ; c1cc(-c2ccc(N(c3ccc(-c4cccc(-n5c6ccccc6c6ccccc65)c4)cc3)c3cccc(-c4cccc5oc6c7ccccc7ccc6c45)c3)cc2)cc(-c2ccc3c(ccc4ccccc43)c2)c1]

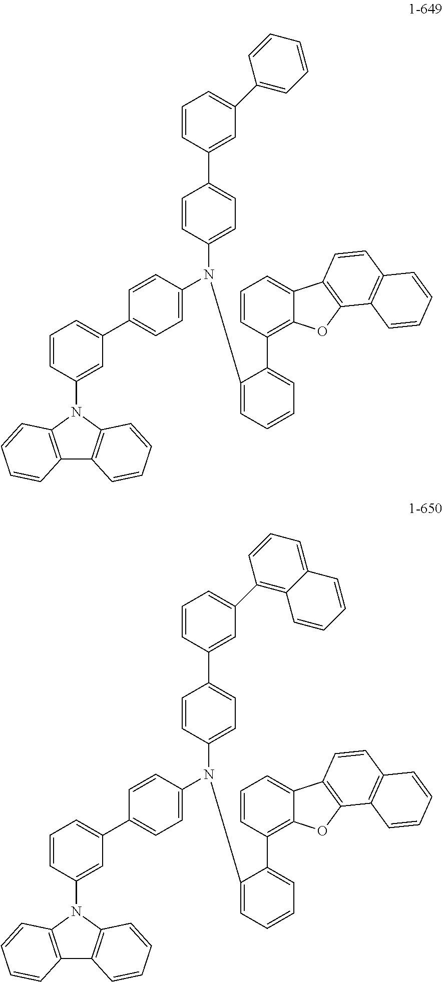 [SMILES: c1ccc(-c2cccc(-c3ccc(N(c4ccc(-c5cccc(-n6c7ccccc7c7ccccc76)c5)cc4)c4ccccc4-c4cccc5c4oc4c6ccccc6ccc54)cc3)c2)cc1 ; c1cc(-c2ccc(N(c3ccc(-c4cccc(-n5c6ccccc6c6ccccc65)c4)cc3)c3ccccc3-c3cccc4c3oc3c5ccccc5ccc43)cc2)cc(-c2cccc3ccccc23)c1]

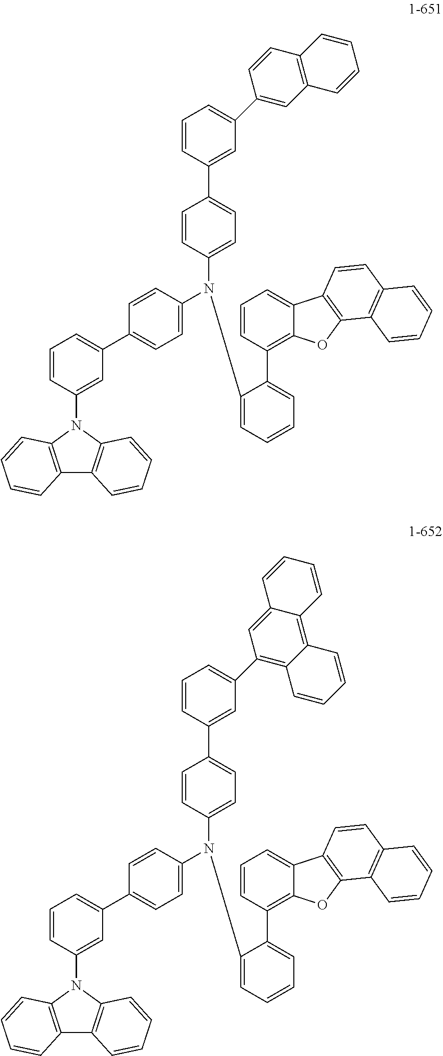 [SMILES: c1cc(-c2ccc(N(c3ccc(-c4cccc(-n5c6ccccc6c6ccccc65)c4)cc3)c3ccccc3-c3cccc4c3oc3c5ccccc5ccc43)cc2)cc(-c2ccc3ccccc3c2)c1 ; c1cc(-c2ccc(N(c3ccc(-c4cccc(-n5c6ccccc6c6ccccc65)c4)cc3)c3ccccc3-c3cccc4c3oc3c5ccccc5ccc43)cc2)cc(-c2cc3ccccc3c3ccccc23)c1]

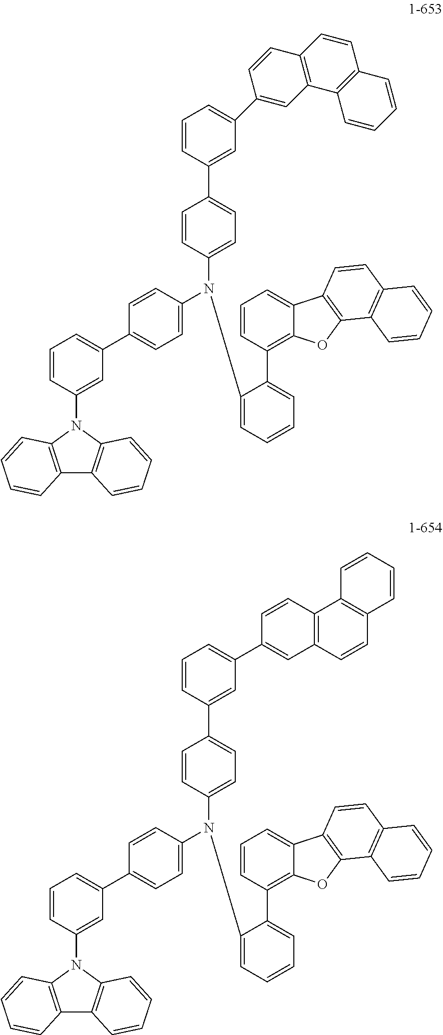 [SMILES: c1cc(-c2ccc(N(c3ccc(-c4cccc(-n5c6ccccc6c6ccccc65)c4)cc3)c3ccccc3-c3cccc4c3oc3c5ccccc5ccc43)cc2)cc(-c2ccc3ccc4ccccc4c3c2)c1 ; c1cc(-c2ccc(N(c3ccc(-c4cccc(-n5c6ccccc6c6ccccc65)c4)cc3)c3ccccc3-c3cccc4c3oc3c5ccccc5ccc43)cc2)cc(-c2ccc3c(ccc4ccccc43)c2)c1]

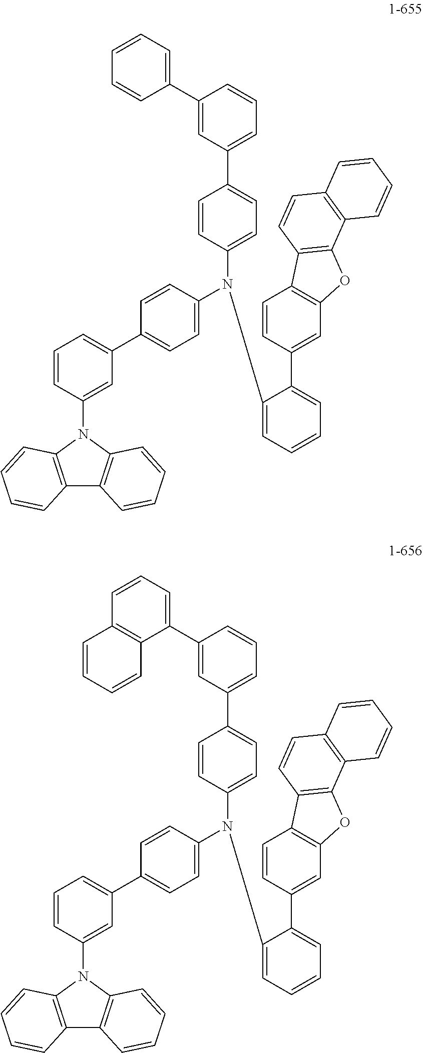 [SMILES: c1ccc(-c2cccc(-c3ccc(N(c4ccc(-c5cccc(-n6c7ccccc7c7ccccc76)c5)cc4)c4ccccc4-c4ccc5c(c4)oc4c6ccccc6ccc54)cc3)c2)cc1 ; c1cc(-c2ccc(N(c3ccc(-c4cccc(-n5c6ccccc6c6ccccc65)c4)cc3)c3ccccc3-c3ccc4c(c3)oc3c5ccccc5ccc43)cc2)cc(-c2cccc3ccccc23)c1]

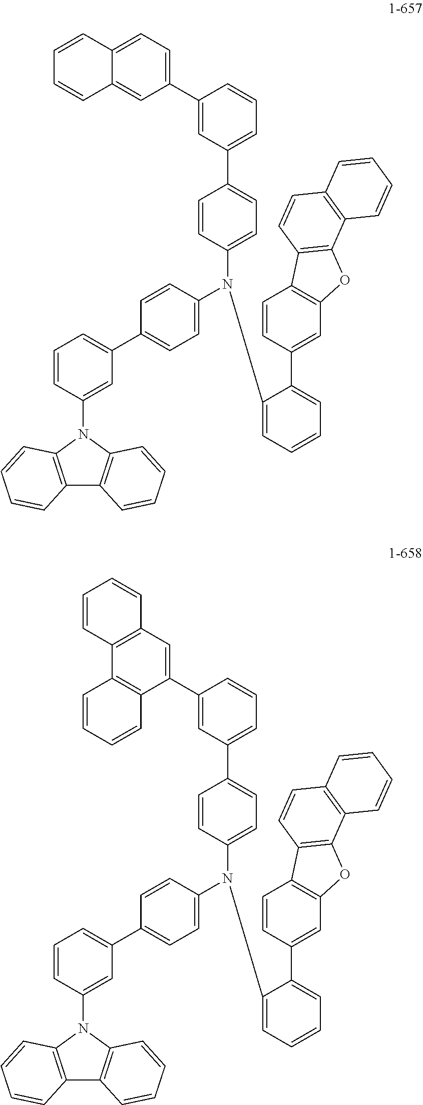 [SMILES: c1cc(-c2ccc(N(c3ccc(-c4cccc(-n5c6ccccc6c6ccccc65)c4)cc3)c3ccccc3-c3ccc4c(c3)oc3c5ccccc5ccc43)cc2)cc(-c2ccc3ccccc3c2)c1 ; c1cc(-c2ccc(N(c3ccc(-c4cccc(-n5c6ccccc6c6ccccc65)c4)cc3)c3ccccc3-c3ccc4c(c3)oc3c5ccccc5ccc43)cc2)cc(-c2cc3ccccc3c3ccccc23)c1]

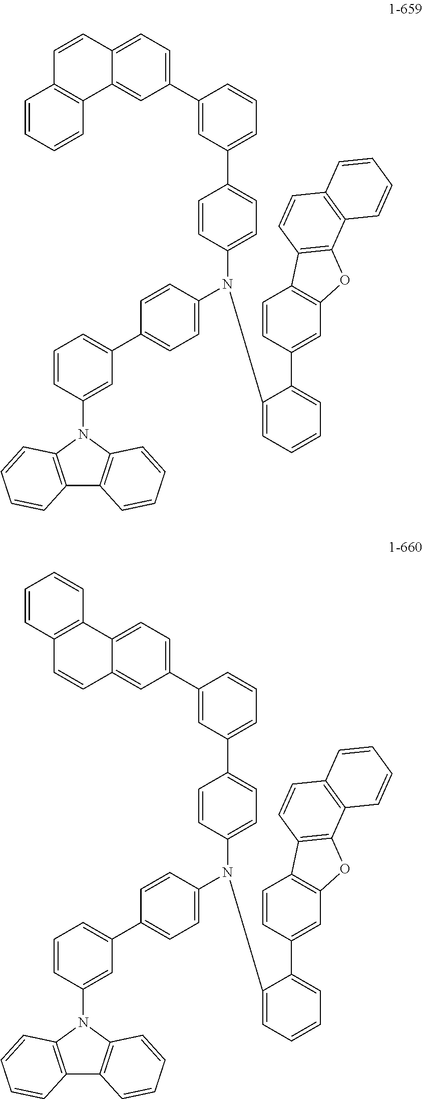 [SMILES: c1cc(-c2ccc(N(c3ccc(-c4cccc(-n5c6ccccc6c6ccccc65)c4)cc3)c3ccccc3-c3ccc4c(c3)oc3c5ccccc5ccc43)cc2)cc(-c2ccc3ccc4ccccc4c3c2)c1 ; c1cc(-c2ccc(N(c3ccc(-c4cccc(-n5c6ccccc6c6ccccc65)c4)cc3)c3ccccc3-c3ccc4c(c3)oc3c5ccccc5ccc43)cc2)cc(-c2ccc3c(ccc4ccccc43)c2)c1]

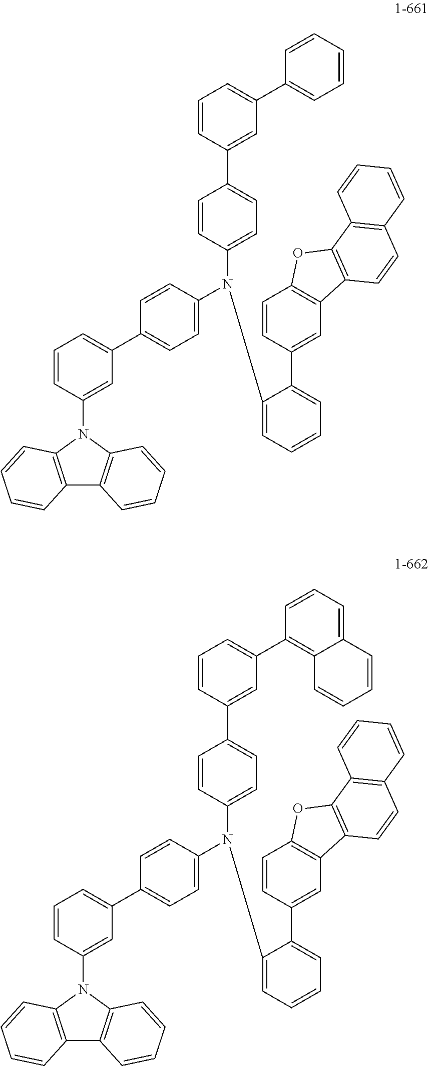 [SMILES: c1ccc(-c2cccc(-c3ccc(N(c4ccc(-c5cccc(-n6c7ccccc7c7ccccc76)c5)cc4)c4ccccc4-c4ccc5oc6c7ccccc7ccc6c5c4)cc3)c2)cc1 ; c1cc(-c2ccc(N(c3ccc(-c4cccc(-n5c6ccccc6c6ccccc65)c4)cc3)c3ccccc3-c3ccc4oc5c6ccccc6ccc5c4c3)cc2)cc(-c2cccc3ccccc23)c1]

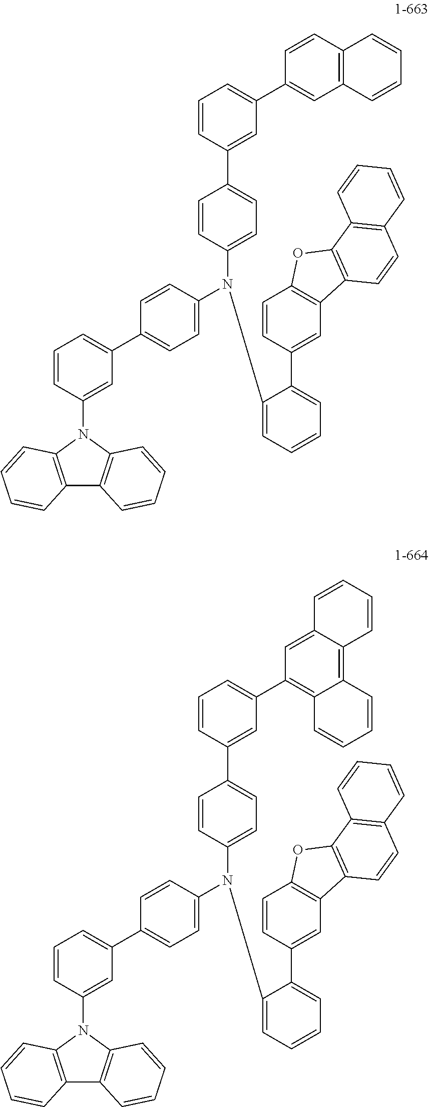 [SMILES: c1cc(-c2ccc(N(c3ccc(-c4cccc(-n5c6ccccc6c6ccccc65)c4)cc3)c3ccccc3-c3ccc4oc5c6ccccc6ccc5c4c3)cc2)cc(-c2ccc3ccccc3c2)c1 ; c1cc(-c2ccc(N(c3ccc(-c4cccc(-n5c6ccccc6c6ccccc65)c4)cc3)c3ccccc3-c3ccc4oc5c6ccccc6ccc5c4c3)cc2)cc(-c2cc3ccccc3c3ccccc23)c1]

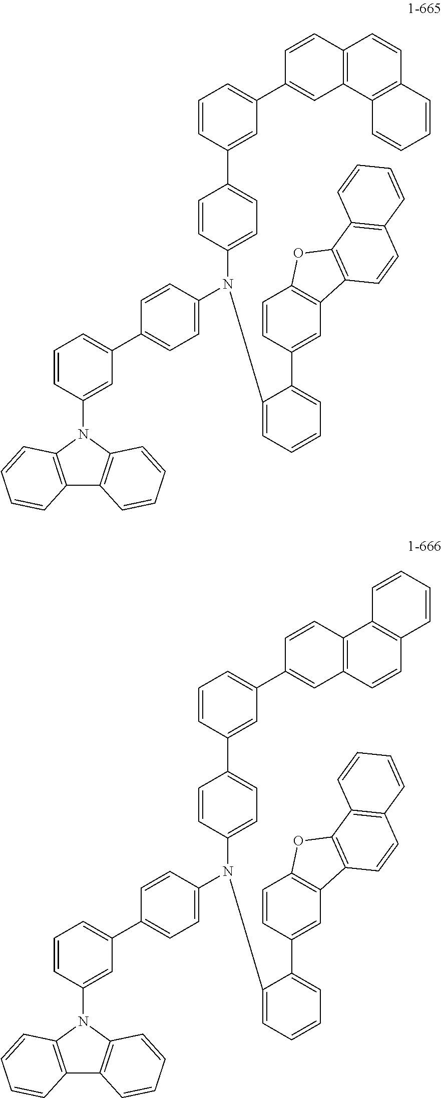 [SMILES: c1cc(-c2ccc(N(c3ccc(-c4cccc(-n5c6ccccc6c6ccccc65)c4)cc3)c3ccccc3-c3ccc4oc5c6ccccc6ccc5c4c3)cc2)cc(-c2ccc3ccc4ccccc4c3c2)c1 ; c1cc(-c2ccc(N(c3ccc(-c4cccc(-n5c6ccccc6c6ccccc65)c4)cc3)c3ccccc3-c3ccc4oc5c6ccccc6ccc5c4c3)cc2)cc(-c2ccc3c(ccc4ccccc43)c2)c1]

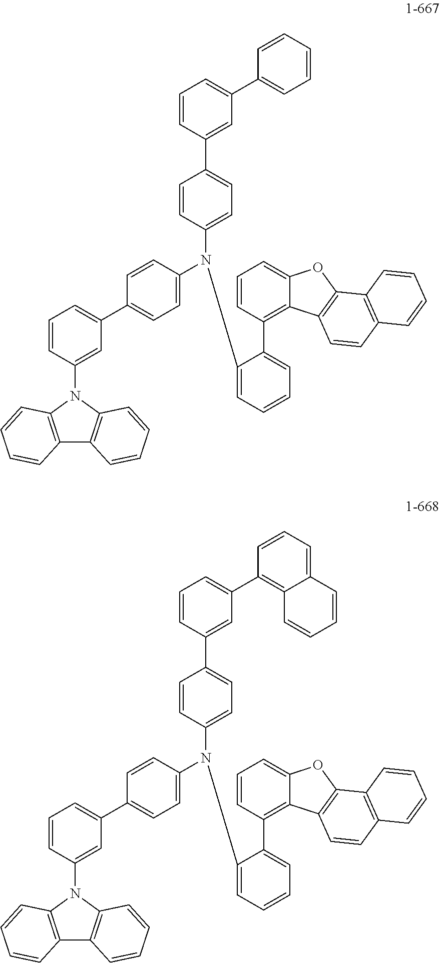 [SMILES: c1ccc(-c2cccc(-c3ccc(N(c4ccc(-c5cccc(-n6c7ccccc7c7ccccc76)c5)cc4)c4ccccc4-c4cccc5oc6c7ccccc7ccc6c45)cc3)c2)cc1 ; c1cc(-c2ccc(N(c3ccc(-c4cccc(-n5c6ccccc6c6ccccc65)c4)cc3)c3ccccc3-c3cccc4oc5c6ccccc6ccc5c34)cc2)cc(-c2cccc3ccccc23)c1]

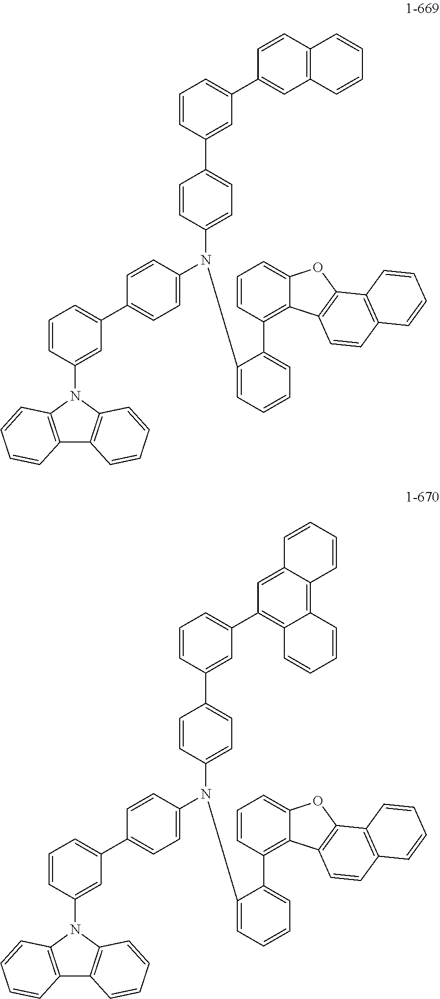 [SMILES: c1cc(-c2ccc(N(c3ccc(-c4cccc(-n5c6ccccc6c6ccccc65)c4)cc3)c3ccccc3-c3cccc4oc5c6ccccc6ccc5c34)cc2)cc(-c2ccc3ccccc3c2)c1 ; c1cc(-c2ccc(N(c3ccc(-c4cccc(-n5c6ccccc6c6ccccc65)c4)cc3)c3ccccc3-c3cccc4oc5c6ccccc6ccc5c34)cc2)cc(-c2cc3ccccc3c3ccccc23)c1]

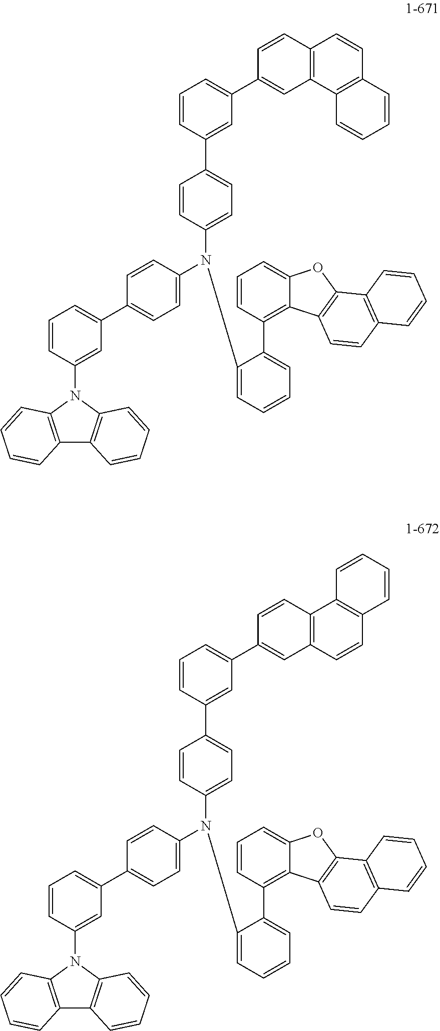 [SMILES: c1cc(-c2ccc(N(c3ccc(-c4cccc(-n5c6ccccc6c6ccccc65)c4)cc3)c3ccccc3-c3cccc4oc5c6ccccc6ccc5c34)cc2)cc(-c2ccc3ccc4ccccc4c3c2)c1 ; c1cc(-c2ccc(N(c3ccc(-c4cccc(-n5c6ccccc6c6ccccc65)c4)cc3)c3ccccc3-c3cccc4oc5c6ccccc6ccc5c34)cc2)cc(-c2ccc3c(ccc4ccccc43)c2)c1]

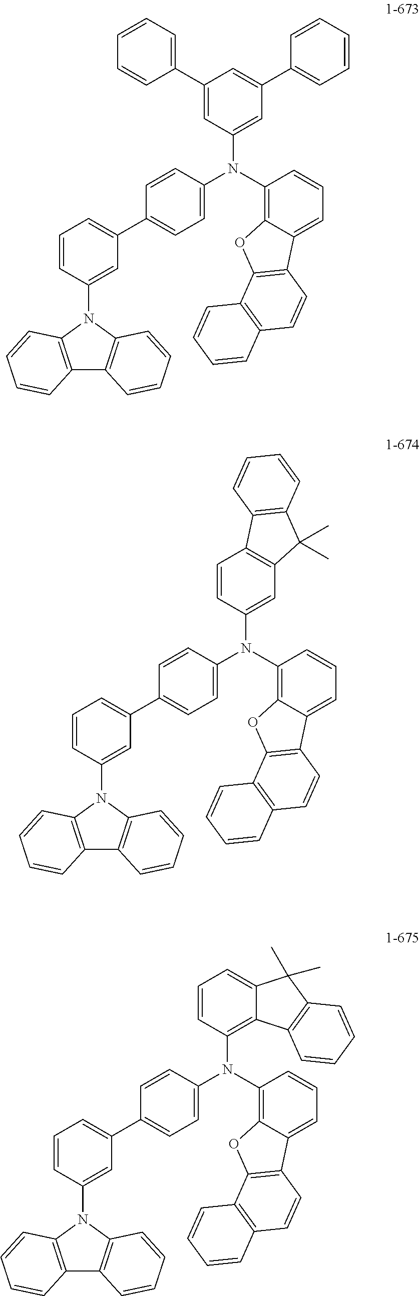 [SMILES: c1ccc(-c2cc(-c3ccccc3)cc(N(c3ccc(-c4cccc(-n5c6ccccc6c6ccccc65)c4)cc3)c3cccc4c3oc3c5ccccc5ccc43)c2)cc1 ; CC1(C)c2ccccc2-c2ccc(N(c3ccc(-c4cccc(-n5c6ccccc6c6ccccc65)c4)cc3)c3cccc4c3oc3c5ccccc5ccc43)cc21 ; CC1(C)c2ccccc2-c2c(N(c3ccc(-c4cccc(-n5c6ccccc6c6ccccc65)c4)cc3)c3cccc4c3oc3c5ccccc5ccc43)cccc21]

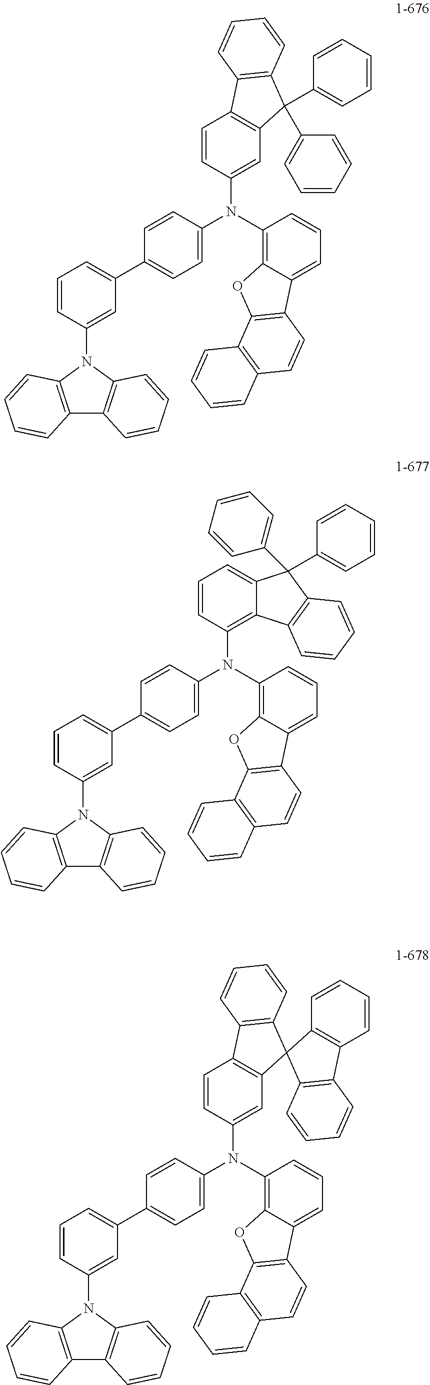 [SMILES: c1ccc(C2(c3ccccc3)c3ccccc3-c3ccc(N(c4ccc(-c5cccc(-n6c7ccccc7c7ccccc76)c5)cc4)c4cccc5c4oc4c6ccccc6ccc54)cc32)cc1 ; c1ccc(C2(c3ccccc3)c3ccccc3-c3c(N(c4ccc(-c5cccc(-n6c7ccccc7c7ccccc76)c5)cc4)c4cccc5c4oc4c6ccccc6ccc54)cccc32)cc1 ; c1cc(-c2ccc(N(c3ccc4c(c3)C3(c5ccccc5-c5ccccc53)c3ccccc3-4)c3cccc4c3oc3c5ccccc5ccc43)cc2)cc(-n2c3ccccc3c3ccccc32)c1]

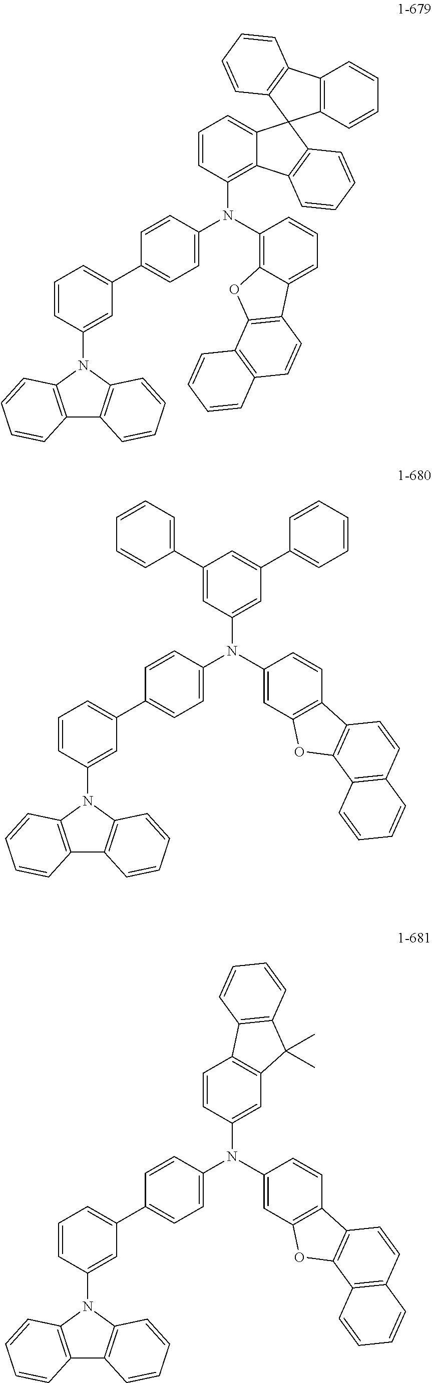 [SMILES: c1cc(-c2ccc(N(c3cccc4c3-c3ccccc3C43c4ccccc4-c4ccccc43)c3cccc4c3oc3c5ccccc5ccc43)cc2)cc(-n2c3ccccc3c3ccccc32)c1 ; c1ccc(-c2cc(-c3ccccc3)cc(N(c3ccc(-c4cccc(-n5c6ccccc6c6ccccc65)c4)cc3)c3ccc4c(c3)oc3c5ccccc5ccc43)c2)cc1 ; CC1(C)c2ccccc2-c2ccc(N(c3ccc(-c4cccc(-n5c6ccccc6c6ccccc65)c4)cc3)c3ccc4c(c3)oc3c5ccccc5ccc43)cc21]

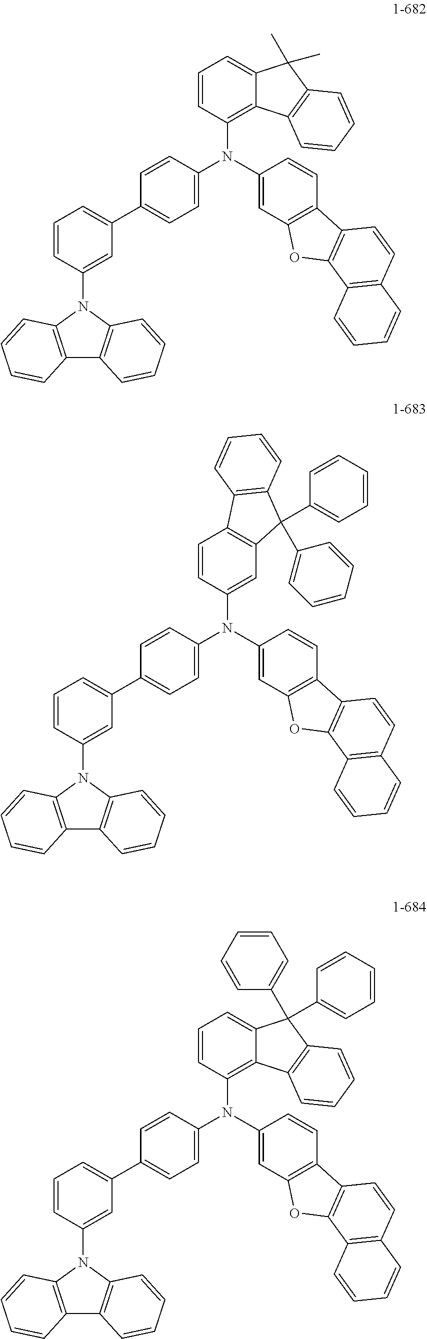 [SMILES: CC1(C)c2ccccc2-c2c(N(c3ccc(-c4cccc(-n5c6ccccc6c6ccccc65)c4)cc3)c3ccc4c(c3)oc3c5ccccc5ccc43)cccc21 ; c1ccc(C2(c3ccccc3)c3ccccc3-c3ccc(N(c4ccc(-c5cccc(-n6c7ccccc7c7ccccc76)c5)cc4)c4ccc5c(c4)oc4c6ccccc6ccc54)cc32)cc1 ; c1ccc(C2(c3ccccc3)c3ccccc3-c3c(N(c4ccc(-c5cccc(-n6c7ccccc7c7ccccc76)c5)cc4)c4ccc5c(c4)oc4c6ccccc6ccc54)cccc32)cc1]

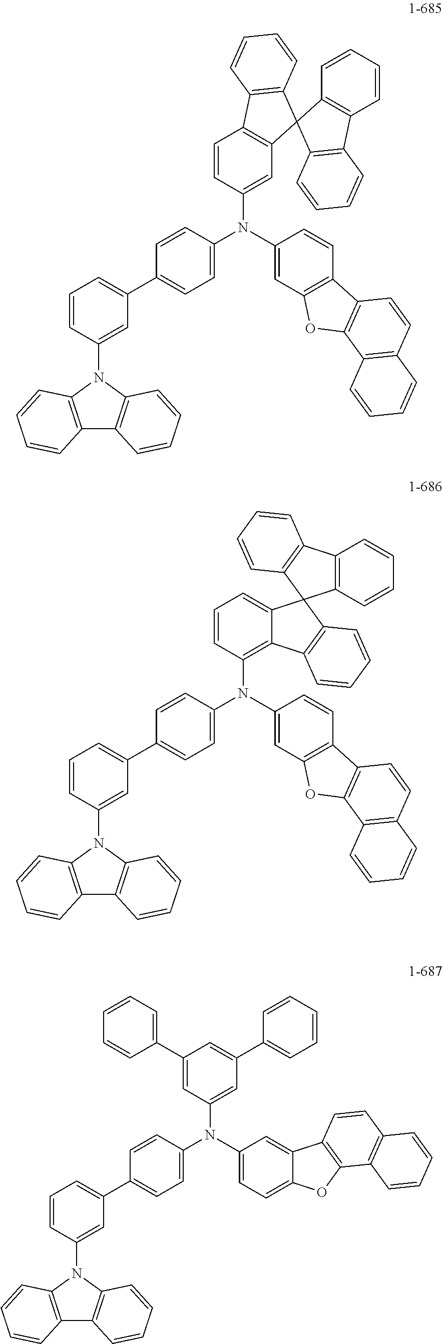 [SMILES: c1cc(-c2ccc(N(c3ccc4c(c3)C3(c5ccccc5-c5ccccc53)c3ccccc3-4)c3ccc4c(c3)oc3c5ccccc5ccc43)cc2)cc(-n2c3ccccc3c3ccccc32)c1 ; c1cc(-c2ccc(N(c3ccc4c(c3)oc3c5ccccc5ccc43)c3cccc4c3-c3ccccc3C43c4ccccc4-c4ccccc43)cc2)cc(-n2c3ccccc3c3ccccc32)c1 ; c1ccc(-c2cc(-c3ccccc3)cc(N(c3ccc(-c4cccc(-n5c6ccccc6c6ccccc65)c4)cc3)c3ccc4oc5c6ccccc6ccc5c4c3)c2)cc1]

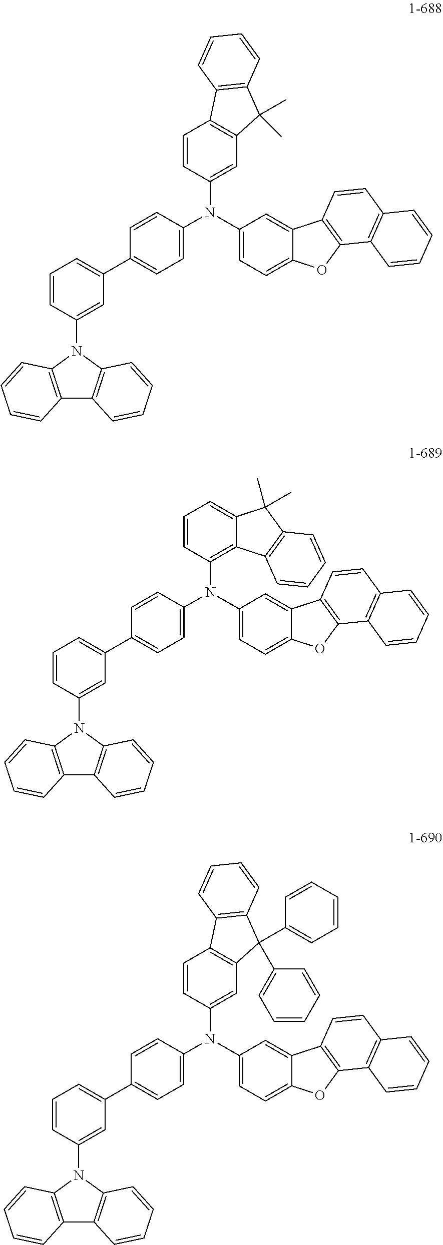 [SMILES: CC1(C)c2ccccc2-c2ccc(N(c3ccc(-c4cccc(-n5c6ccccc6c6ccccc65)c4)cc3)c3ccc4oc5c6ccccc6ccc5c4c3)cc21 ; CC1(C)c2ccccc2-c2c(N(c3ccc(-c4cccc(-n5c6ccccc6c6ccccc65)c4)cc3)c3ccc4oc5c6ccccc6ccc5c4c3)cccc21 ; c1ccc(C2(c3ccccc3)c3ccccc3-c3ccc(N(c4ccc(-c5cccc(-n6c7ccccc7c7ccccc76)c5)cc4)c4ccc5oc6c7ccccc7ccc6c5c4)cc32)cc1]

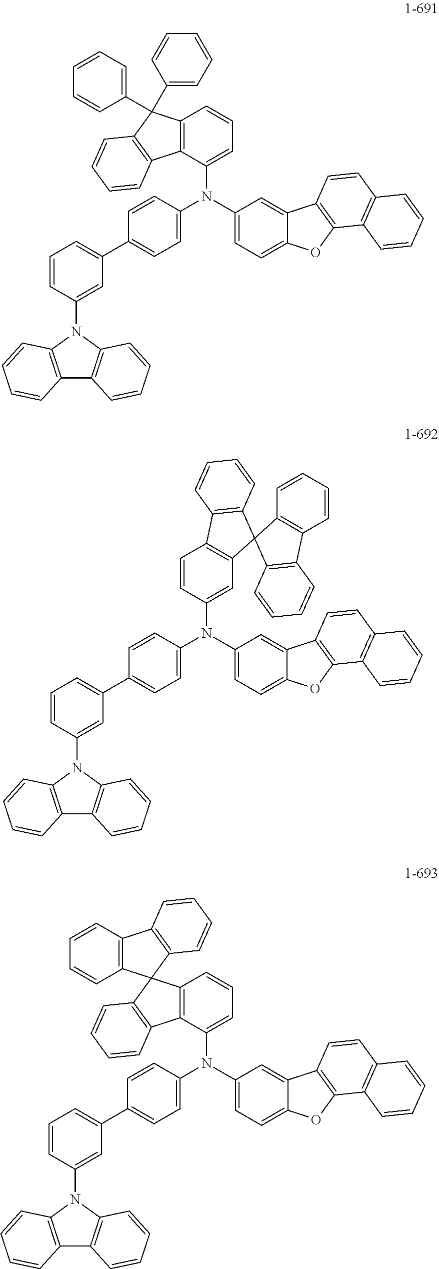 [SMILES: c1ccc(C2(c3ccccc3)c3ccccc3-c3c(N(c4ccc(-c5cccc(-n6c7ccccc7c7ccccc76)c5)cc4)c4ccc5oc6c7ccccc7ccc6c5c4)cccc32)cc1 ; c1cc(-c2ccc(N(c3ccc4c(c3)C3(c5ccccc5-c5ccccc53)c3ccccc3-4)c3ccc4oc5c6ccccc6ccc5c4c3)cc2)cc(-n2c3ccccc3c3ccccc32)c1 ; c1cc(-c2ccc(N(c3ccc4oc5c6ccccc6ccc5c4c3)c3cccc4c3-c3ccccc3C43c4ccccc4-c4ccccc43)cc2)cc(-n2c3ccccc3c3ccccc32)c1]

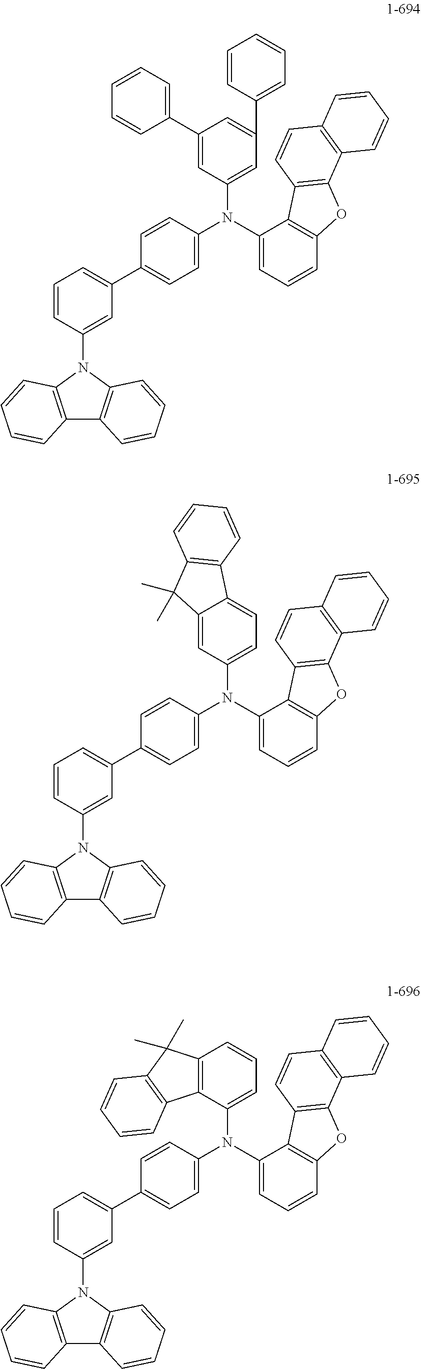 [SMILES: c1ccc(-c2cc(-c3ccccc3)cc(N(c3ccc(-c4cccc(-n5c6ccccc6c6ccccc65)c4)cc3)c3cccc4oc5c6ccccc6ccc5c34)c2)cc1 ; CC1(C)c2ccccc2-c2ccc(N(c3ccc(-c4cccc(-n5c6ccccc6c6ccccc65)c4)cc3)c3cccc4oc5c6ccccc6ccc5c34)cc21 ; CC1(C)c2ccccc2-c2c(N(c3ccc(-c4cccc(-n5c6ccccc6c6ccccc65)c4)cc3)c3cccc4oc5c6ccccc6ccc5c34)cccc21]

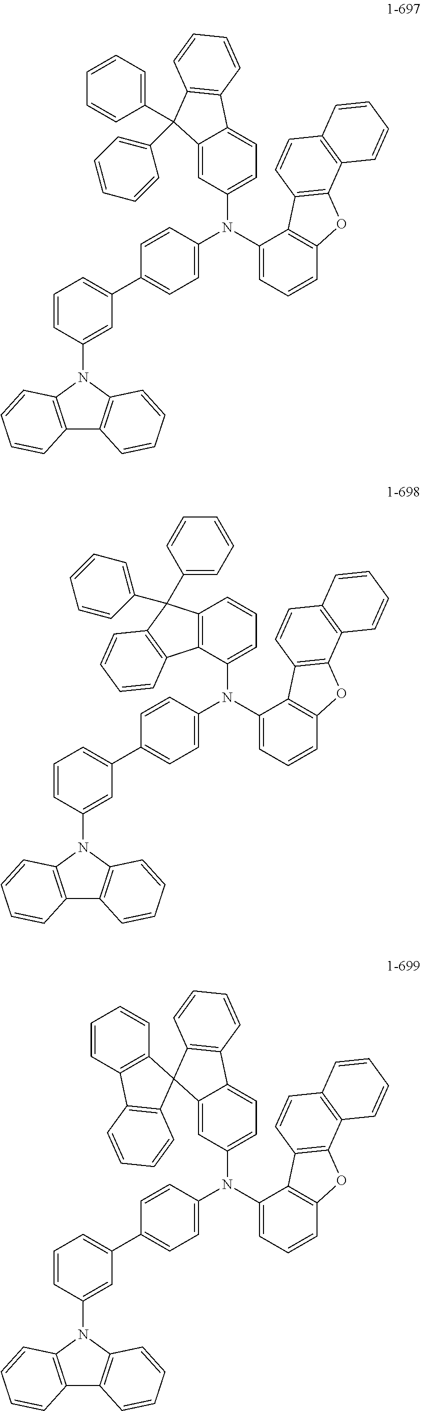 [SMILES: c1ccc(C2(c3ccccc3)c3ccccc3-c3ccc(N(c4ccc(-c5cccc(-n6c7ccccc7c7ccccc76)c5)cc4)c4cccc5oc6c7ccccc7ccc6c45)cc32)cc1 ; c1ccc(C2(c3ccccc3)c3ccccc3-c3c(N(c4ccc(-c5cccc(-n6c7ccccc7c7ccccc76)c5)cc4)c4cccc5oc6c7ccccc7ccc6c45)cccc32)cc1 ; c1cc(-c2ccc(N(c3ccc4c(c3)C3(c5ccccc5-c5ccccc53)c3ccccc3-4)c3cccc4oc5c6ccccc6ccc5c34)cc2)cc(-n2c3ccccc3c3ccccc32)c1]

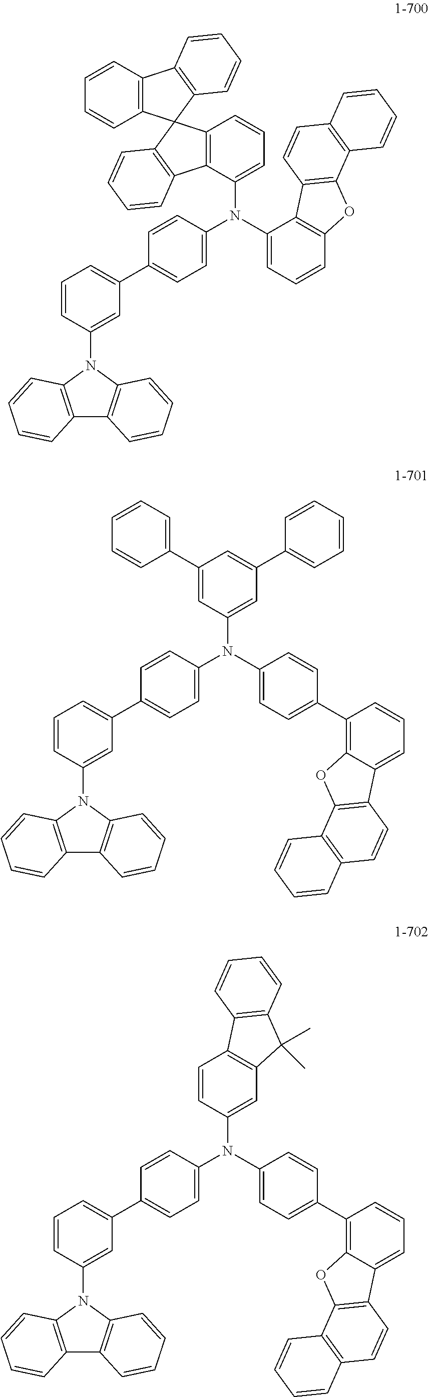 [SMILES: c1cc(-c2ccc(N(c3cccc4c3-c3ccccc3C43c4ccccc4-c4ccccc43)c3cccc4oc5c6ccccc6ccc5c34)cc2)cc(-n2c3ccccc3c3ccccc32)c1 ; c1ccc(-c2cc(-c3ccccc3)cc(N(c3ccc(-c4cccc(-n5c6ccccc6c6ccccc65)c4)cc3)c3ccc(-c4cccc5c4oc4c6ccccc6ccc54)cc3)c2)cc1 ; CC1(C)c2ccccc2-c2ccc(N(c3ccc(-c4cccc(-n5c6ccccc6c6ccccc65)c4)cc3)c3ccc(-c4cccc5c4oc4c6ccccc6ccc54)cc3)cc21]

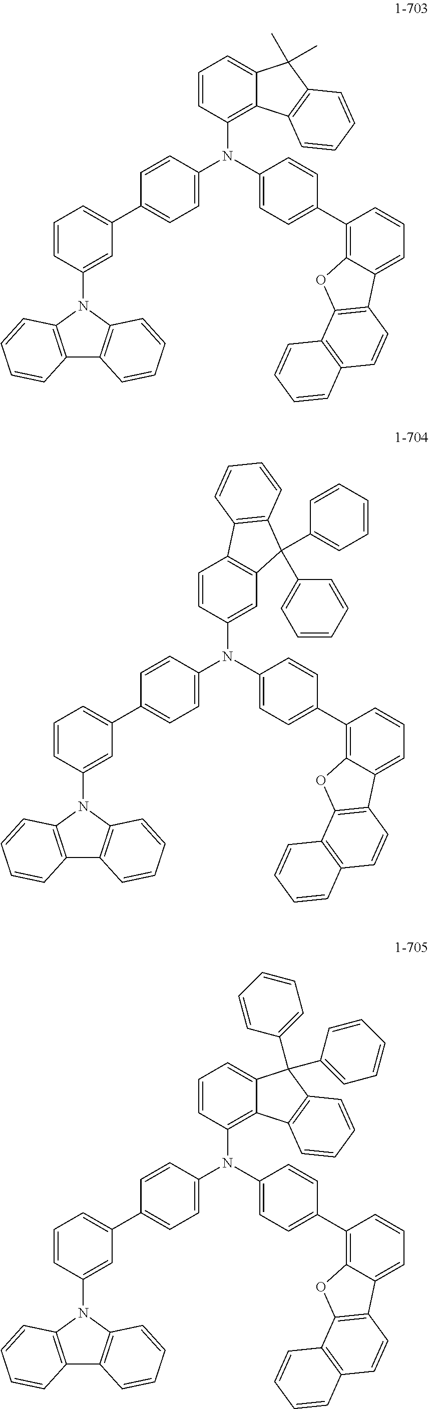 [SMILES: CC1(C)c2ccccc2-c2c(N(c3ccc(-c4cccc(-n5c6ccccc6c6ccccc65)c4)cc3)c3ccc(-c4cccc5c4oc4c6ccccc6ccc54)cc3)cccc21 ; c1ccc(C2(c3ccccc3)c3ccccc3-c3ccc(N(c4ccc(-c5cccc(-n6c7ccccc7c7ccccc76)c5)cc4)c4ccc(-c5cccc6c5oc5c7ccccc7ccc65)cc4)cc32)cc1 ; c1ccc(C2(c3ccccc3)c3ccccc3-c3c(N(c4ccc(-c5cccc(-n6c7ccccc7c7ccccc76)c5)cc4)c4ccc(-c5cccc6c5oc5c7ccccc7ccc65)cc4)cccc32)cc1]

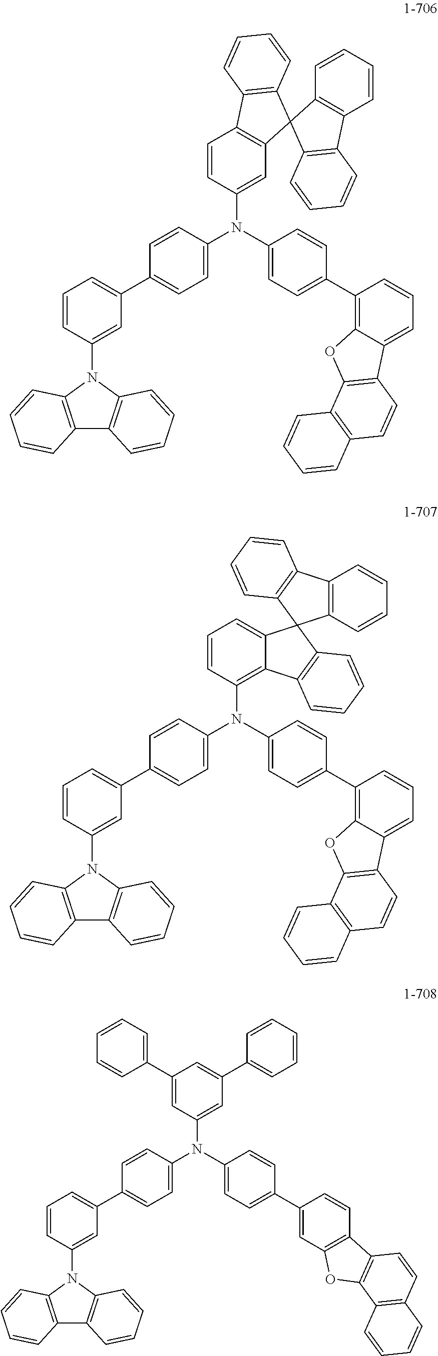 [SMILES: c1cc(-c2ccc(N(c3ccc(-c4cccc5c4oc4c6ccccc6ccc54)cc3)c3ccc4c(c3)C3(c5ccccc5-c5ccccc53)c3ccccc3-4)cc2)cc(-n2c3ccccc3c3ccccc32)c1 ; c1cc(-c2ccc(N(c3ccc(-c4cccc5c4oc4c6ccccc6ccc54)cc3)c3cccc4c3-c3ccccc3C43c4ccccc4-c4ccccc43)cc2)cc(-n2c3ccccc3c3ccccc32)c1 ; c1ccc(-c2cc(-c3ccccc3)cc(N(c3ccc(-c4cccc(-n5c6ccccc6c6ccccc65)c4)cc3)c3ccc(-c4ccc5c(c4)oc4c6ccccc6ccc54)cc3)c2)cc1]

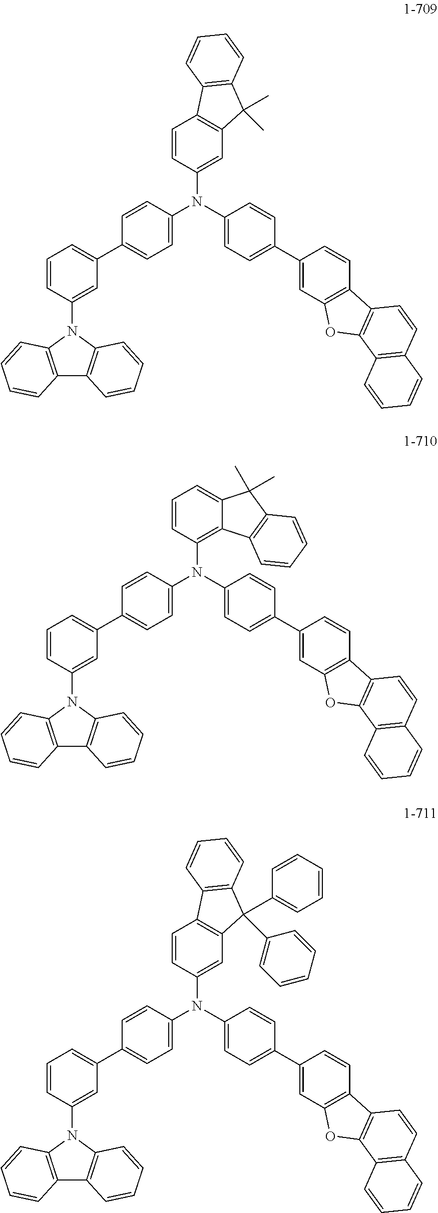 [SMILES: CC1(C)c2ccccc2-c2ccc(N(c3ccc(-c4cccc(-n5c6ccccc6c6ccccc65)c4)cc3)c3ccc(-c4ccc5c(c4)oc4c6ccccc6ccc54)cc3)cc21 ; CC1(C)c2ccccc2-c2c(N(c3ccc(-c4cccc(-n5c6ccccc6c6ccccc65)c4)cc3)c3ccc(-c4ccc5c(c4)oc4c6ccccc6ccc54)cc3)cccc21 ; c1ccc(C2(c3ccccc3)c3ccccc3-c3ccc(N(c4ccc(-c5cccc(-n6c7ccccc7c7ccccc76)c5)cc4)c4ccc(-c5ccc6c(c5)oc5c7ccccc7ccc65)cc4)cc32)cc1]

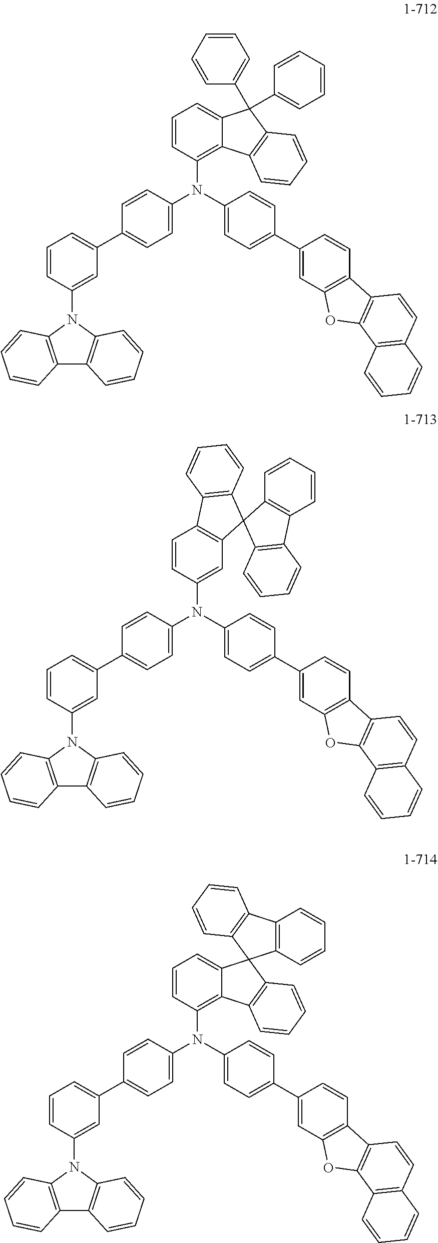 [SMILES: c1ccc(C2(c3ccccc3)c3ccccc3-c3c(N(c4ccc(-c5cccc(-n6c7ccccc7c7ccccc76)c5)cc4)c4ccc(-c5ccc6c(c5)oc5c7ccccc7ccc65)cc4)cccc32)cc1 ; c1cc(-c2ccc(N(c3ccc(-c4ccc5c(c4)oc4c6ccccc6ccc54)cc3)c3ccc4c(c3)C3(c5ccccc5-c5ccccc53)c3ccccc3-4)cc2)cc(-n2c3ccccc3c3ccccc32)c1 ; c1cc(-c2ccc(N(c3ccc(-c4ccc5c(c4)oc4c6ccccc6ccc54)cc3)c3cccc4c3-c3ccccc3C43c4ccccc4-c4ccccc43)cc2)cc(-n2c3ccccc3c3ccccc32)c1]

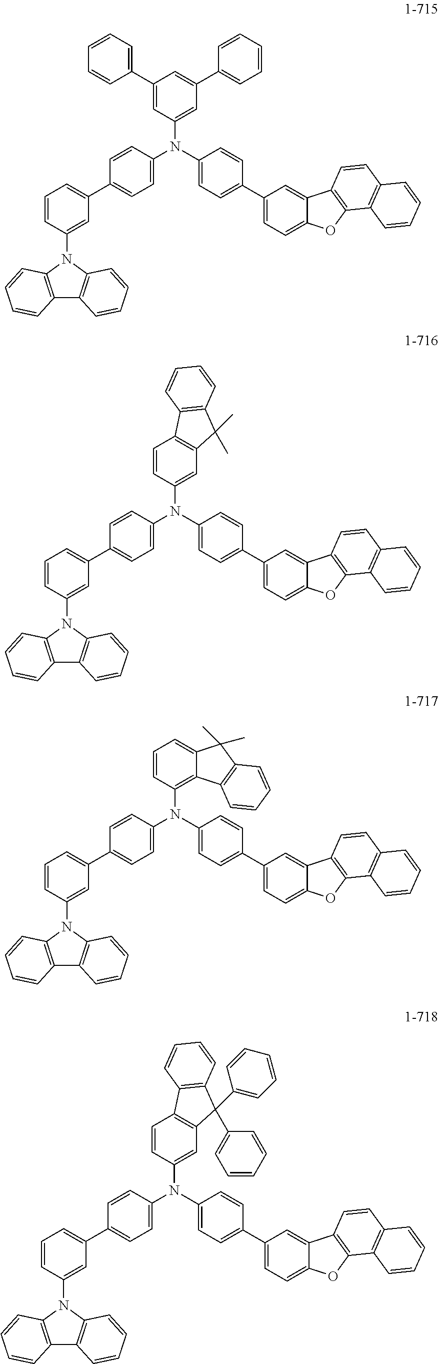 [SMILES: c1ccc(-c2cc(-c3ccccc3)cc(N(c3ccc(-c4cccc(-n5c6ccccc6c6ccccc65)c4)cc3)c3ccc(-c4ccc5oc6c7ccccc7ccc6c5c4)cc3)c2)cc1 ; CC1(C)c2ccccc2-c2ccc(N(c3ccc(-c4cccc(-n5c6ccccc6c6ccccc65)c4)cc3)c3ccc(-c4ccc5oc6c7ccccc7ccc6c5c4)cc3)cc21 ; CC1(C)c2ccccc2-c2c(N(c3ccc(-c4cccc(-n5c6ccccc6c6ccccc65)c4)cc3)c3ccc(-c4ccc5oc6c7ccccc7ccc6c5c4)cc3)cccc21 ; c1ccc(C2(c3ccccc3)c3ccccc3-c3ccc(N(c4ccc(-c5cccc(-n6c7ccccc7c7ccccc76)c5)cc4)c4ccc(-c5ccc6oc7c8ccccc8ccc7c6c5)cc4)cc32)cc1]

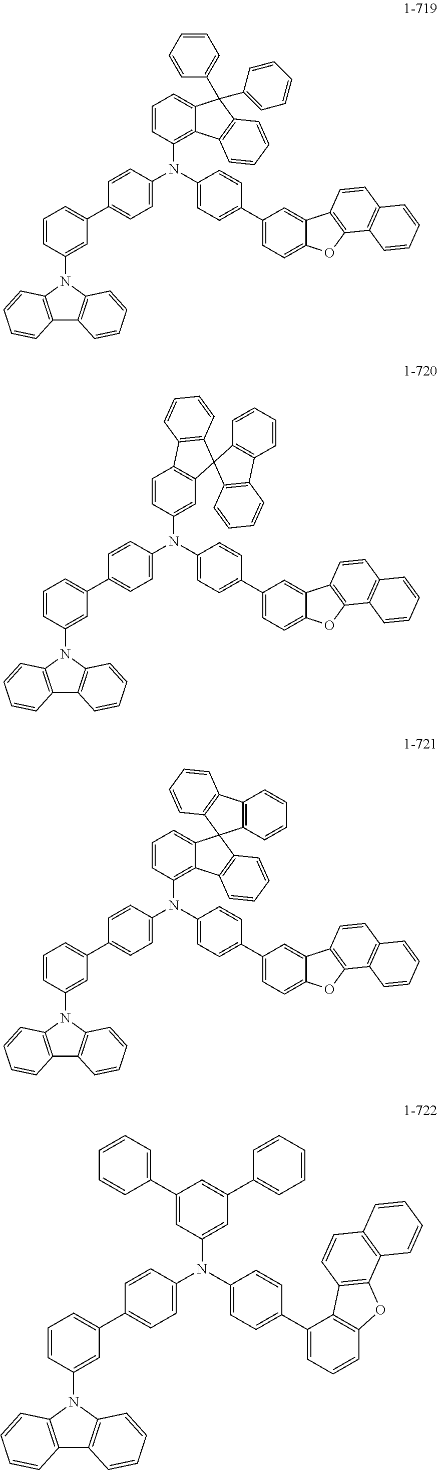 [SMILES: c1ccc(C2(c3ccccc3)c3ccccc3-c3c(N(c4ccc(-c5cccc(-n6c7ccccc7c7ccccc76)c5)cc4)c4ccc(-c5ccc6oc7c8ccccc8ccc7c6c5)cc4)cccc32)cc1 ; c1cc(-c2ccc(N(c3ccc(-c4ccc5oc6c7ccccc7ccc6c5c4)cc3)c3ccc4c(c3)C3(c5ccccc5-c5ccccc53)c3ccccc3-4)cc2)cc(-n2c3ccccc3c3ccccc32)c1 ; c1cc(-c2ccc(N(c3ccc(-c4ccc5oc6c7ccccc7ccc6c5c4)cc3)c3cccc4c3-c3ccccc3C43c4ccccc4-c4ccccc43)cc2)cc(-n2c3ccccc3c3ccccc32)c1 ; c1ccc(-c2cc(-c3ccccc3)cc(N(c3ccc(-c4cccc(-n5c6ccccc6c6ccccc65)c4)cc3)c3ccc(-c4cccc5oc6c7ccccc7ccc6c45)cc3)c2)cc1]

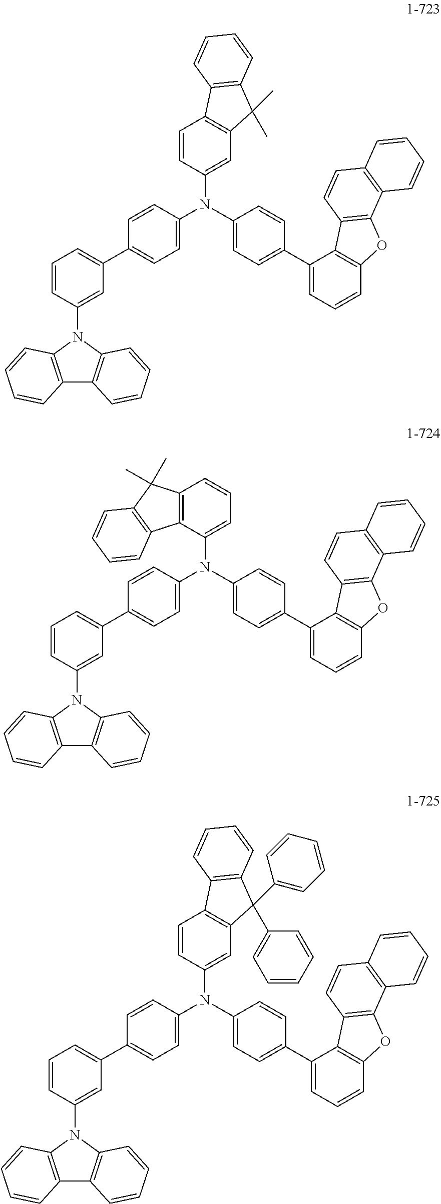 [SMILES: CC1(C)c2ccccc2-c2ccc(N(c3ccc(-c4cccc(-n5c6ccccc6c6ccccc65)c4)cc3)c3ccc(-c4cccc5oc6c7ccccc7ccc6c45)cc3)cc21 ; CC1(C)c2ccccc2-c2c(N(c3ccc(-c4cccc(-n5c6ccccc6c6ccccc65)c4)cc3)c3ccc(-c4cccc5oc6c7ccccc7ccc6c45)cc3)cccc21 ; c1ccc(C2(c3ccccc3)c3ccccc3-c3ccc(N(c4ccc(-c5cccc(-n6c7ccccc7c7ccccc76)c5)cc4)c4ccc(-c5cccc6oc7c8ccccc8ccc7c56)cc4)cc32)cc1]

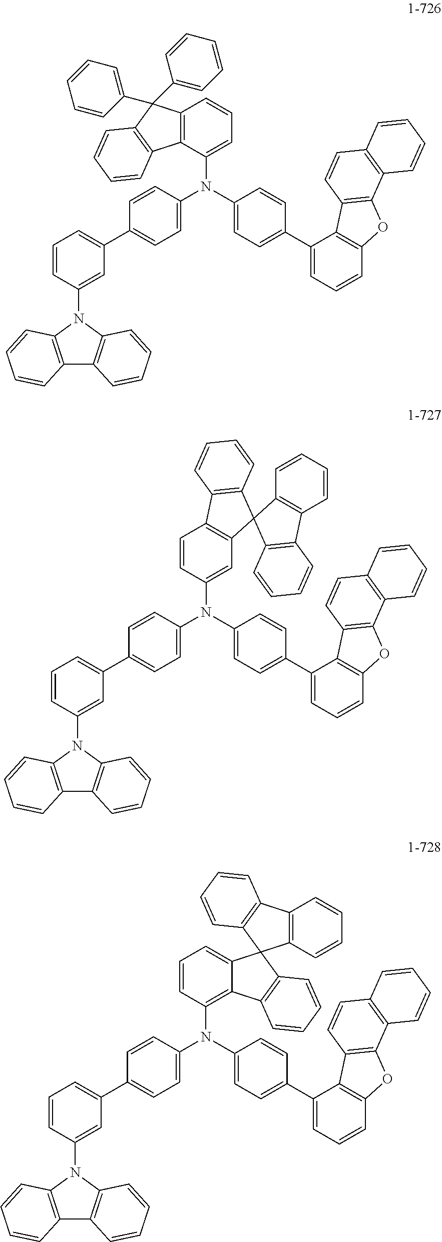 [SMILES: c1ccc(C2(c3ccccc3)c3ccccc3-c3c(N(c4ccc(-c5cccc(-n6c7ccccc7c7ccccc76)c5)cc4)c4ccc(-c5cccc6oc7c8ccccc8ccc7c56)cc4)cccc32)cc1 ; c1cc(-c2ccc(N(c3ccc(-c4cccc5oc6c7ccccc7ccc6c45)cc3)c3ccc4c(c3)C3(c5ccccc5-c5ccccc53)c3ccccc3-4)cc2)cc(-n2c3ccccc3c3ccccc32)c1 ; c1cc(-c2ccc(N(c3ccc(-c4cccc5oc6c7ccccc7ccc6c45)cc3)c3cccc4c3-c3ccccc3C43c4ccccc4-c4ccccc43)cc2)cc(-n2c3ccccc3c3ccccc32)c1]

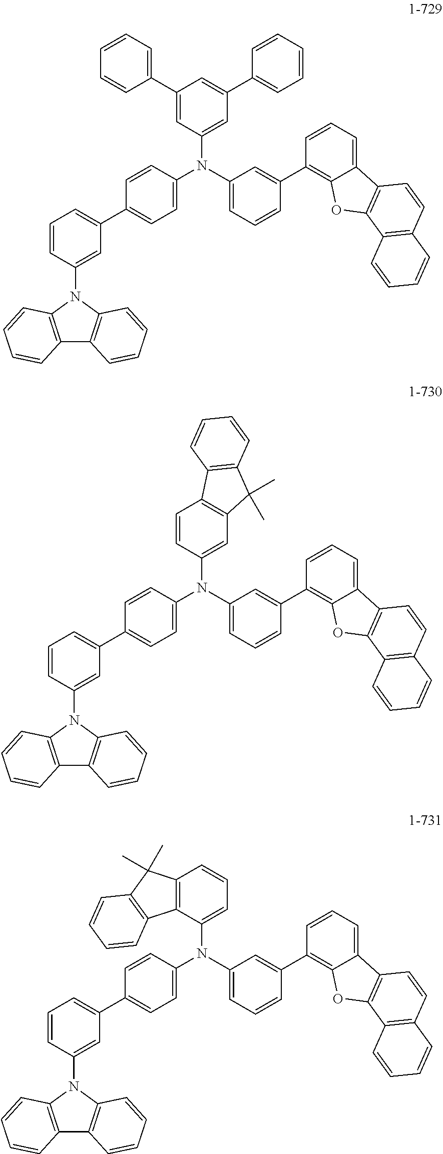 [SMILES: c1ccc(-c2cc(-c3ccccc3)cc(N(c3ccc(-c4cccc(-n5c6ccccc6c6ccccc65)c4)cc3)c3cccc(-c4cccc5c4oc4c6ccccc6ccc54)c3)c2)cc1 ; CC1(C)c2ccccc2-c2ccc(N(c3ccc(-c4cccc(-n5c6ccccc6c6ccccc65)c4)cc3)c3cccc(-c4cccc5c4oc4c6ccccc6ccc54)c3)cc21 ; CC1(C)c2ccccc2-c2c(N(c3ccc(-c4cccc(-n5c6ccccc6c6ccccc65)c4)cc3)c3cccc(-c4cccc5c4oc4c6ccccc6ccc54)c3)cccc21]

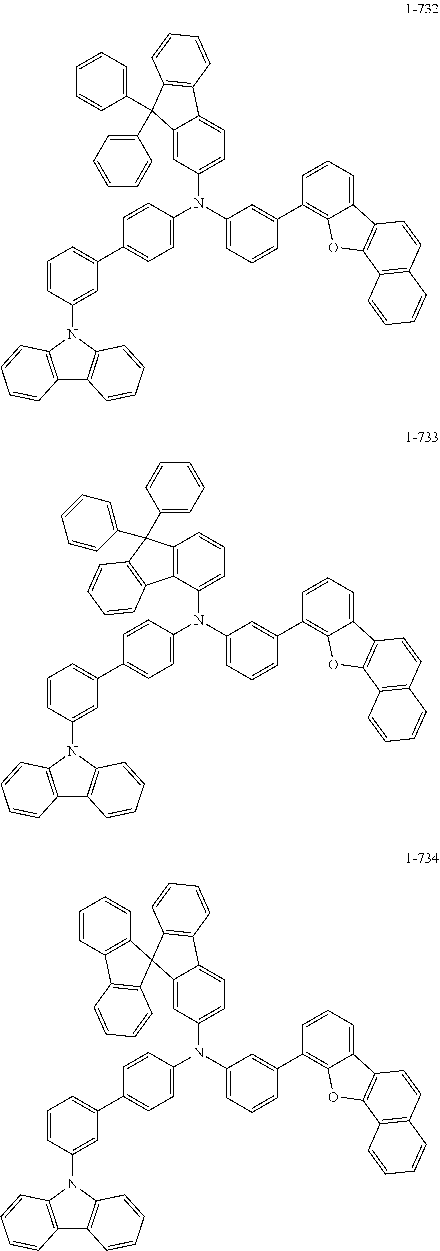 [SMILES: c1ccc(C2(c3ccccc3)c3ccccc3-c3ccc(N(c4ccc(-c5cccc(-n6c7ccccc7c7ccccc76)c5)cc4)c4cccc(-c5cccc6c5oc5c7ccccc7ccc65)c4)cc32)cc1 ; c1ccc(C2(c3ccccc3)c3ccccc3-c3c(N(c4ccc(-c5cccc(-n6c7ccccc7c7ccccc76)c5)cc4)c4cccc(-c5cccc6c5oc5c7ccccc7ccc65)c4)cccc32)cc1 ; c1cc(-c2cccc3c2oc2c4ccccc4ccc32)cc(N(c2ccc(-c3cccc(-n4c5ccccc5c5ccccc54)c3)cc2)c2ccc3c(c2)C2(c4ccccc4-c4ccccc42)c2ccccc2-3)c1]

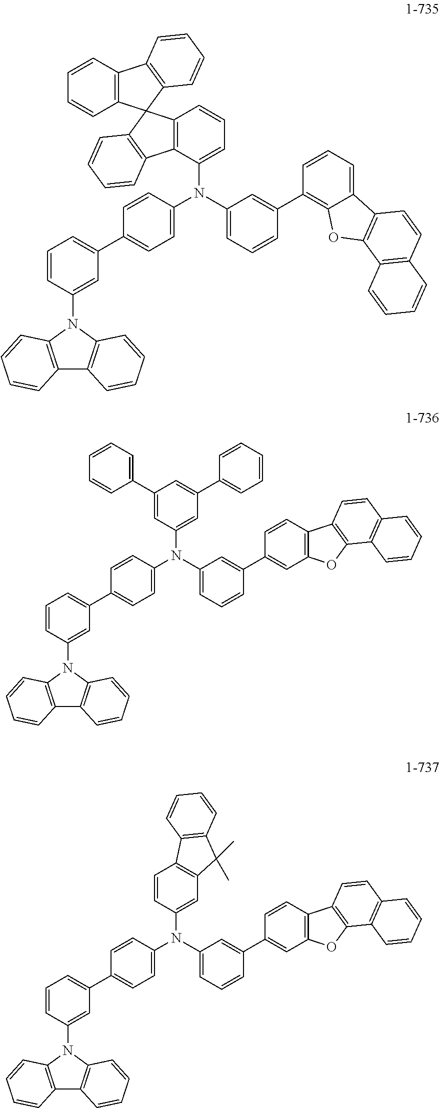 [SMILES: c1cc(-c2cccc3c2oc2c4ccccc4ccc32)cc(N(c2ccc(-c3cccc(-n4c5ccccc5c5ccccc54)c3)cc2)c2cccc3c2-c2ccccc2C32c3ccccc3-c3ccccc32)c1 ; c1ccc(-c2cc(-c3ccccc3)cc(N(c3ccc(-c4cccc(-n5c6ccccc6c6ccccc65)c4)cc3)c3cccc(-c4ccc5c(c4)oc4c6ccccc6ccc54)c3)c2)cc1 ; CC1(C)c2ccccc2-c2ccc(N(c3ccc(-c4cccc(-n5c6ccccc6c6ccccc65)c4)cc3)c3cccc(-c4ccc5c(c4)oc4c6ccccc6ccc54)c3)cc21]

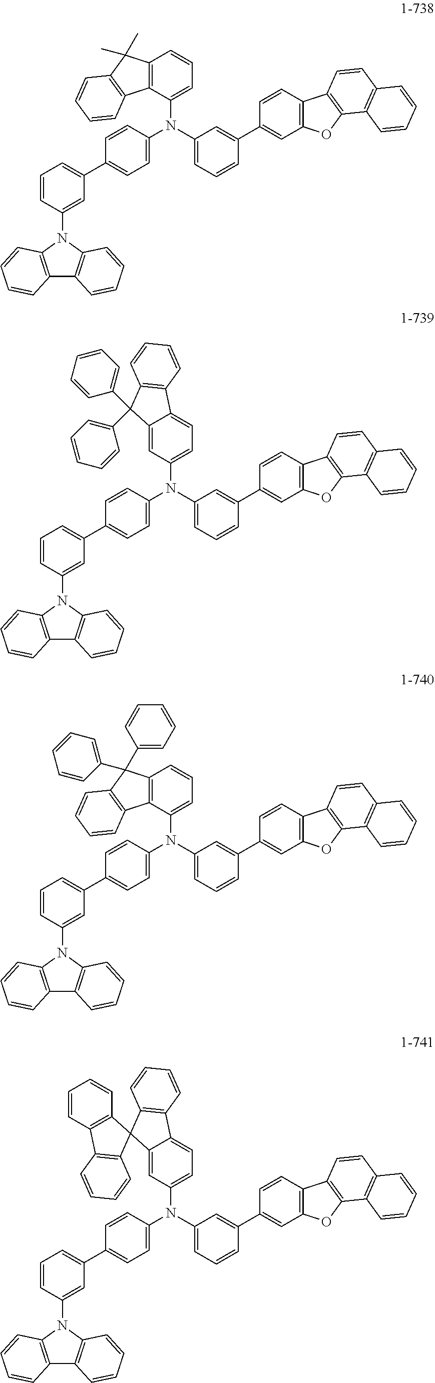 [SMILES: CC1(C)c2ccccc2-c2c(N(c3ccc(-c4cccc(-n5c6ccccc6c6ccccc65)c4)cc3)c3cccc(-c4ccc5c(c4)oc4c6ccccc6ccc54)c3)cccc21 ; c1ccc(C2(c3ccccc3)c3ccccc3-c3ccc(N(c4ccc(-c5cccc(-n6c7ccccc7c7ccccc76)c5)cc4)c4cccc(-c5ccc6c(c5)oc5c7ccccc7ccc65)c4)cc32)cc1 ; c1ccc(C2(c3ccccc3)c3ccccc3-c3c(N(c4ccc(-c5cccc(-n6c7ccccc7c7ccccc76)c5)cc4)c4cccc(-c5ccc6c(c5)oc5c7ccccc7ccc65)c4)cccc32)cc1 ; c1cc(-c2ccc3c(c2)oc2c4ccccc4ccc32)cc(N(c2ccc(-c3cccc(-n4c5ccccc5c5ccccc54)c3)cc2)c2ccc3c(c2)C2(c4ccccc4-c4ccccc42)c2ccccc2-3)c1]

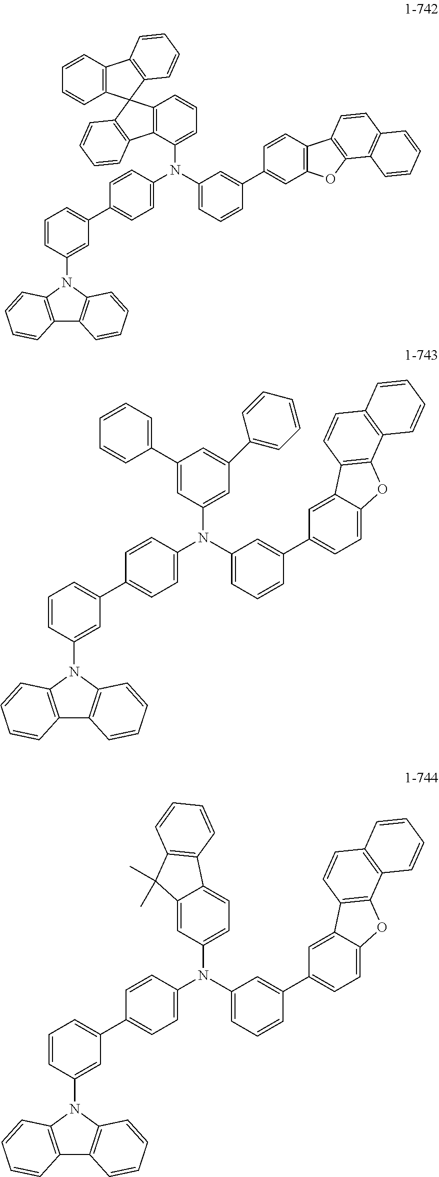 [SMILES: c1cc(-c2ccc3c(c2)oc2c4ccccc4ccc32)cc(N(c2ccc(-c3cccc(-n4c5ccccc5c5ccccc54)c3)cc2)c2cccc3c2-c2ccccc2C32c3ccccc3-c3ccccc32)c1 ; c1ccc(-c2cc(-c3ccccc3)cc(N(c3ccc(-c4cccc(-n5c6ccccc6c6ccccc65)c4)cc3)c3cccc(-c4ccc5oc6c7ccccc7ccc6c5c4)c3)c2)cc1 ; CC1(C)c2ccccc2-c2ccc(N(c3ccc(-c4cccc(-n5c6ccccc6c6ccccc65)c4)cc3)c3cccc(-c4ccc5oc6c7ccccc7ccc6c5c4)c3)cc21]

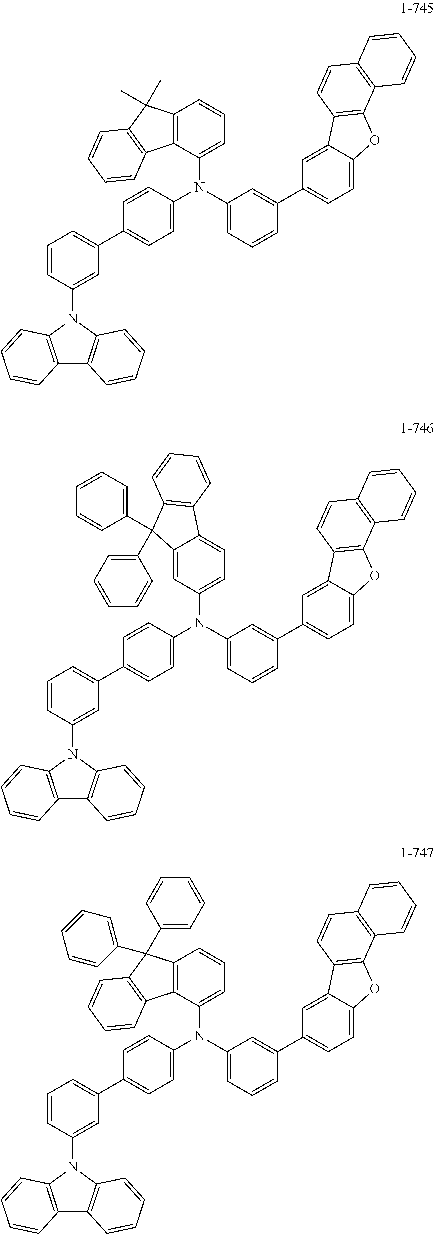 [SMILES: CC1(C)c2ccccc2-c2c(N(c3ccc(-c4cccc(-n5c6ccccc6c6ccccc65)c4)cc3)c3cccc(-c4ccc5oc6c7ccccc7ccc6c5c4)c3)cccc21 ; c1ccc(C2(c3ccccc3)c3ccccc3-c3ccc(N(c4ccc(-c5cccc(-n6c7ccccc7c7ccccc76)c5)cc4)c4cccc(-c5ccc6oc7c8ccccc8ccc7c6c5)c4)cc32)cc1 ; c1ccc(C2(c3ccccc3)c3ccccc3-c3c(N(c4ccc(-c5cccc(-n6c7ccccc7c7ccccc76)c5)cc4)c4cccc(-c5ccc6oc7c8ccccc8ccc7c6c5)c4)cccc32)cc1]

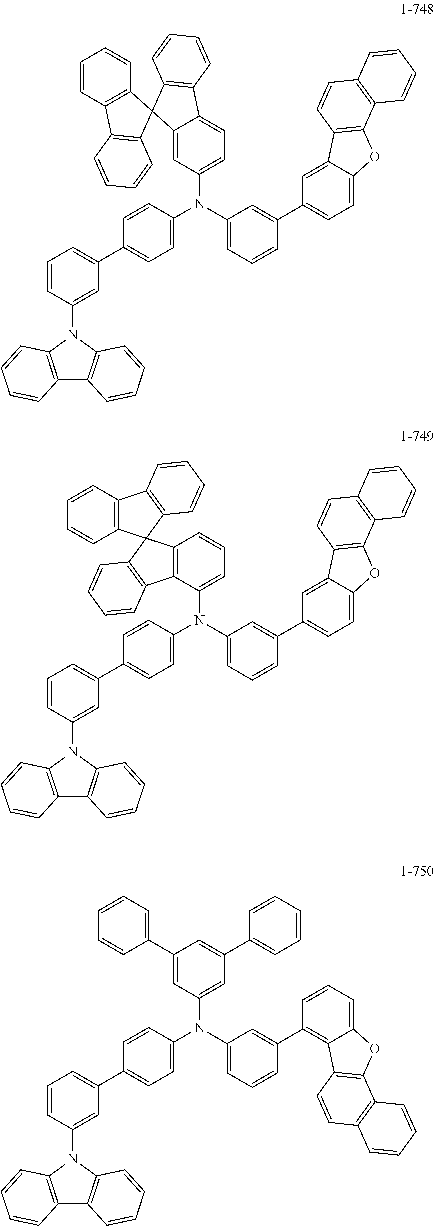 [SMILES: c1cc(-c2ccc3oc4c5ccccc5ccc4c3c2)cc(N(c2ccc(-c3cccc(-n4c5ccccc5c5ccccc54)c3)cc2)c2ccc3c(c2)C2(c4ccccc4-c4ccccc42)c2ccccc2-3)c1 ; c1cc(-c2ccc3oc4c5ccccc5ccc4c3c2)cc(N(c2ccc(-c3cccc(-n4c5ccccc5c5ccccc54)c3)cc2)c2cccc3c2-c2ccccc2C32c3ccccc3-c3ccccc32)c1 ; c1ccc(-c2cc(-c3ccccc3)cc(N(c3ccc(-c4cccc(-n5c6ccccc6c6ccccc65)c4)cc3)c3cccc(-c4cccc5oc6c7ccccc7ccc6c45)c3)c2)cc1]

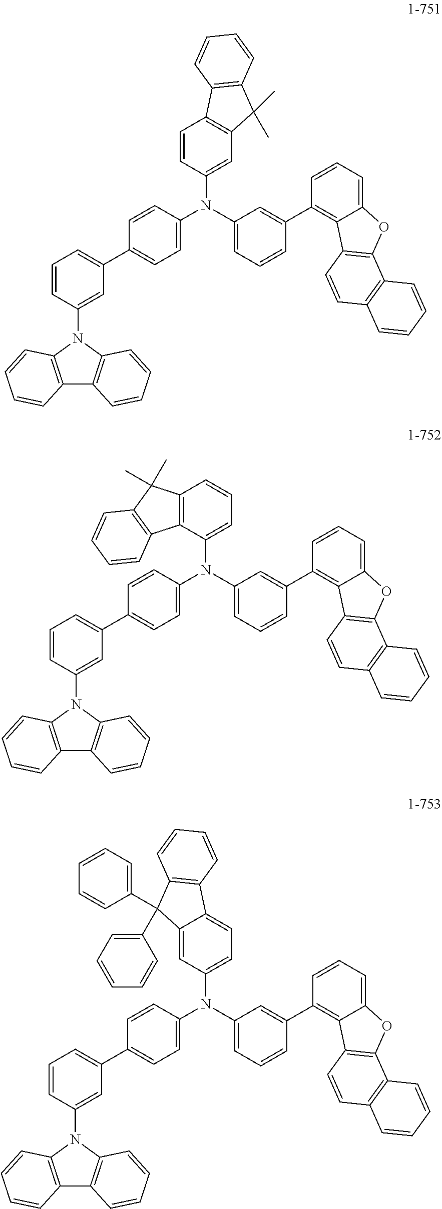 [SMILES: CC1(C)c2ccccc2-c2ccc(N(c3ccc(-c4cccc(-n5c6ccccc6c6ccccc65)c4)cc3)c3cccc(-c4cccc5oc6c7ccccc7ccc6c45)c3)cc21 ; CC1(C)c2ccccc2-c2c(N(c3ccc(-c4cccc(-n5c6ccccc6c6ccccc65)c4)cc3)c3cccc(-c4cccc5oc6c7ccccc7ccc6c45)c3)cccc21 ; c1ccc(C2(c3ccccc3)c3ccccc3-c3ccc(N(c4ccc(-c5cccc(-n6c7ccccc7c7ccccc76)c5)cc4)c4cccc(-c5cccc6oc7c8ccccc8ccc7c56)c4)cc32)cc1]

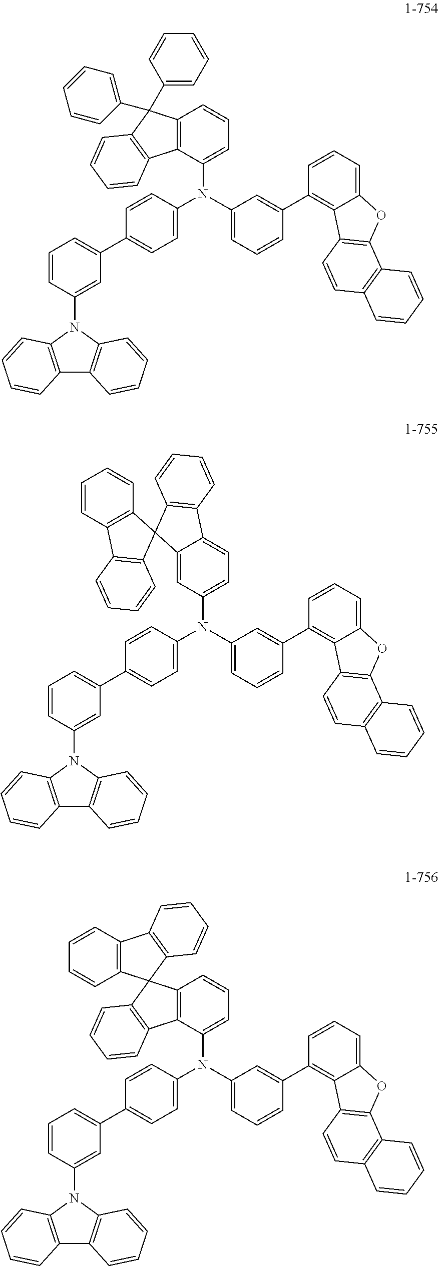 [SMILES: c1ccc(C2(c3ccccc3)c3ccccc3-c3c(N(c4ccc(-c5cccc(-n6c7ccccc7c7ccccc76)c5)cc4)c4cccc(-c5cccc6oc7c8ccccc8ccc7c56)c4)cccc32)cc1 ; c1cc(-c2cccc3oc4c5ccccc5ccc4c23)cc(N(c2ccc(-c3cccc(-n4c5ccccc5c5ccccc54)c3)cc2)c2ccc3c(c2)C2(c4ccccc4-c4ccccc42)c2ccccc2-3)c1 ; c1cc(-c2cccc3oc4c5ccccc5ccc4c23)cc(N(c2ccc(-c3cccc(-n4c5ccccc5c5ccccc54)c3)cc2)c2cccc3c2-c2ccccc2C32c3ccccc3-c3ccccc32)c1]

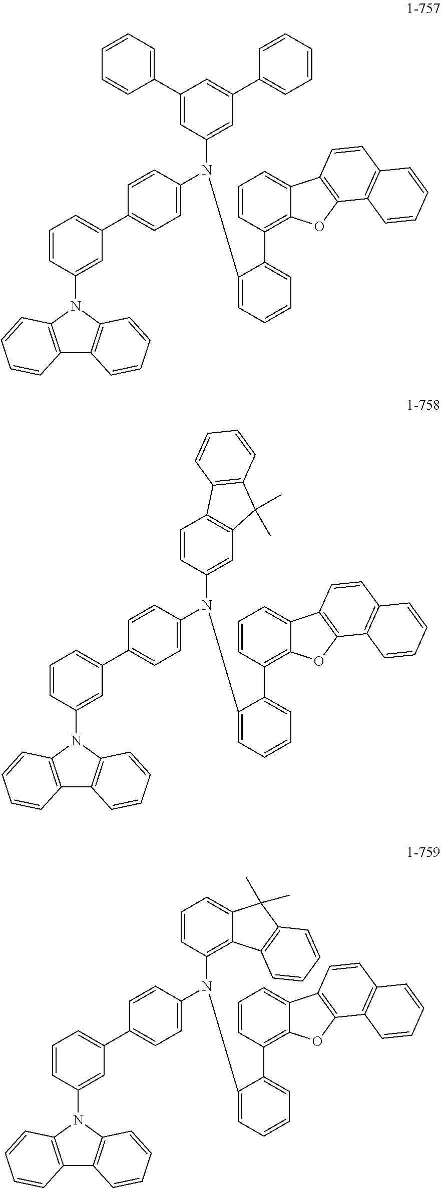 [SMILES: c1ccc(-c2cc(-c3ccccc3)cc(N(c3ccc(-c4cccc(-n5c6ccccc6c6ccccc65)c4)cc3)c3ccccc3-c3cccc4c3oc3c5ccccc5ccc43)c2)cc1 ; CC1(C)c2ccccc2-c2ccc(N(c3ccc(-c4cccc(-n5c6ccccc6c6ccccc65)c4)cc3)c3ccccc3-c3cccc4c3oc3c5ccccc5ccc43)cc21 ; CC1(C)c2ccccc2-c2c(N(c3ccc(-c4cccc(-n5c6ccccc6c6ccccc65)c4)cc3)c3ccccc3-c3cccc4c3oc3c5ccccc5ccc43)cccc21]

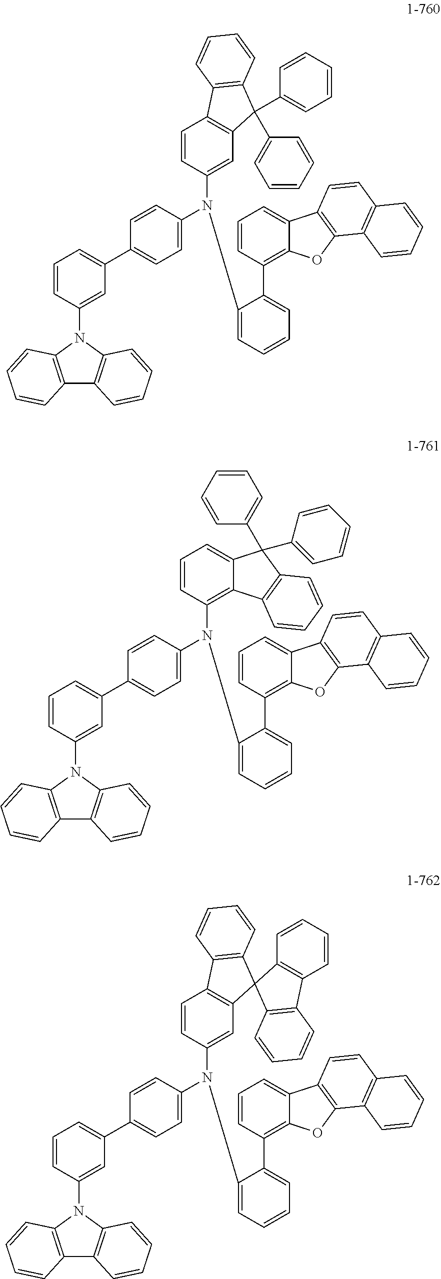 [SMILES: c1ccc(C2(c3ccccc3)c3ccccc3-c3ccc(N(c4ccc(-c5cccc(-n6c7ccccc7c7ccccc76)c5)cc4)c4ccccc4-c4cccc5c4oc4c6ccccc6ccc54)cc32)cc1 ; c1ccc(C2(c3ccccc3)c3ccccc3-c3c(N(c4ccc(-c5cccc(-n6c7ccccc7c7ccccc76)c5)cc4)c4ccccc4-c4cccc5c4oc4c6ccccc6ccc54)cccc32)cc1 ; c1cc(-c2ccc(N(c3ccc4c(c3)C3(c5ccccc5-c5ccccc53)c3ccccc3-4)c3ccccc3-c3cccc4c3oc3c5ccccc5ccc43)cc2)cc(-n2c3ccccc3c3ccccc32)c1]

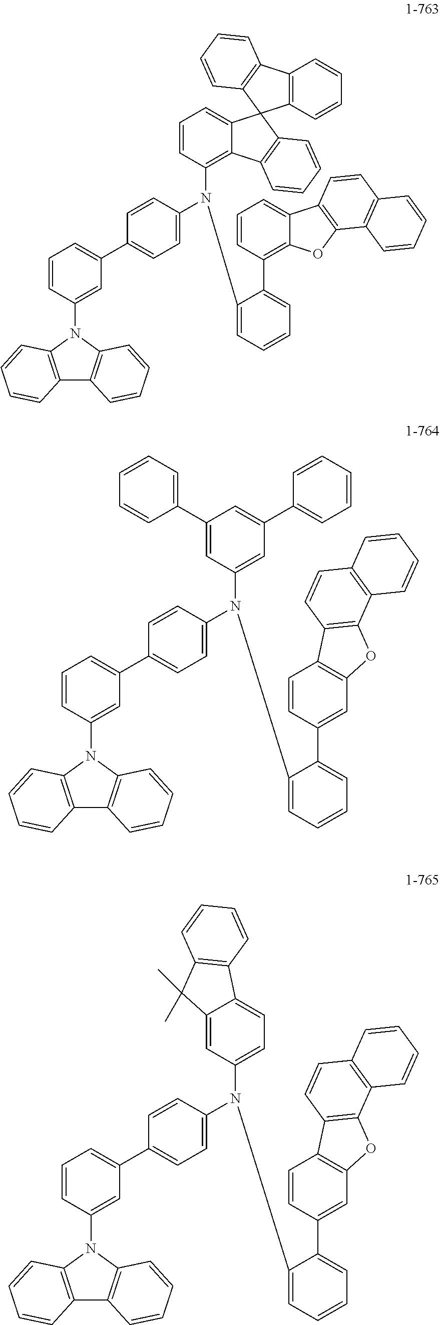 [SMILES: c1cc(-c2ccc(N(c3ccccc3-c3cccc4c3oc3c5ccccc5ccc43)c3cccc4c3-c3ccccc3C43c4ccccc4-c4ccccc43)cc2)cc(-n2c3ccccc3c3ccccc32)c1 ; c1ccc(-c2cc(-c3ccccc3)cc(N(c3ccc(-c4cccc(-n5c6ccccc6c6ccccc65)c4)cc3)c3ccccc3-c3ccc4c(c3)oc3c5ccccc5ccc43)c2)cc1 ; CC1(C)c2ccccc2-c2ccc(N(c3ccc(-c4cccc(-n5c6ccccc6c6ccccc65)c4)cc3)c3ccccc3-c3ccc4c(c3)oc3c5ccccc5ccc43)cc21]

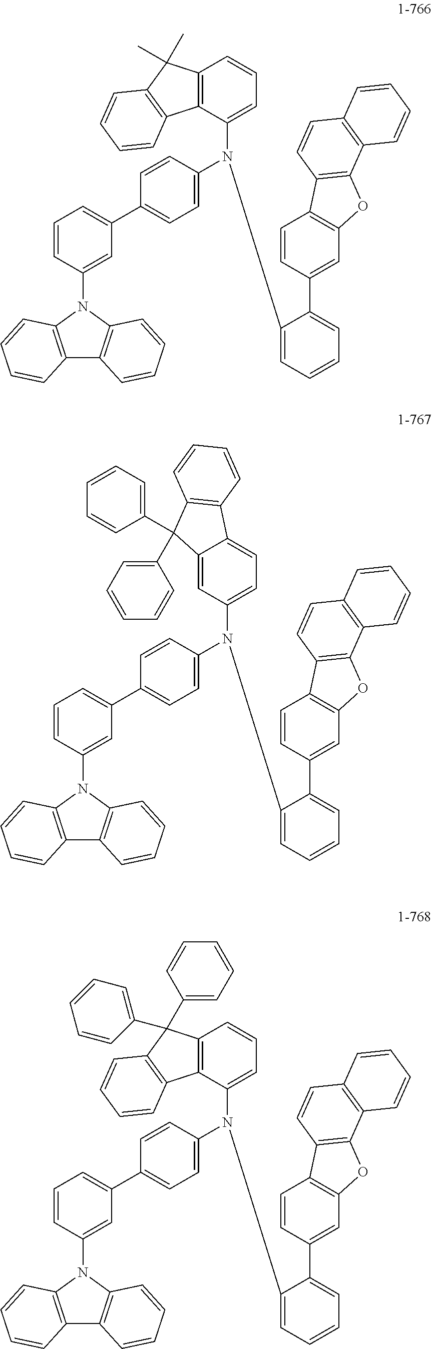 [SMILES: CC1(C)c2ccccc2-c2c(N(c3ccc(-c4cccc(-n5c6ccccc6c6ccccc65)c4)cc3)c3ccccc3-c3ccc4c(c3)oc3c5ccccc5ccc43)cccc21 ; c1ccc(C2(c3ccccc3)c3ccccc3-c3ccc(N(c4ccc(-c5cccc(-n6c7ccccc7c7ccccc76)c5)cc4)c4ccccc4-c4ccc5c(c4)oc4c6ccccc6ccc54)cc32)cc1 ; c1ccc(C2(c3ccccc3)c3ccccc3-c3c(N(c4ccc(-c5cccc(-n6c7ccccc7c7ccccc76)c5)cc4)c4ccccc4-c4ccc5c(c4)oc4c6ccccc6ccc54)cccc32)cc1]

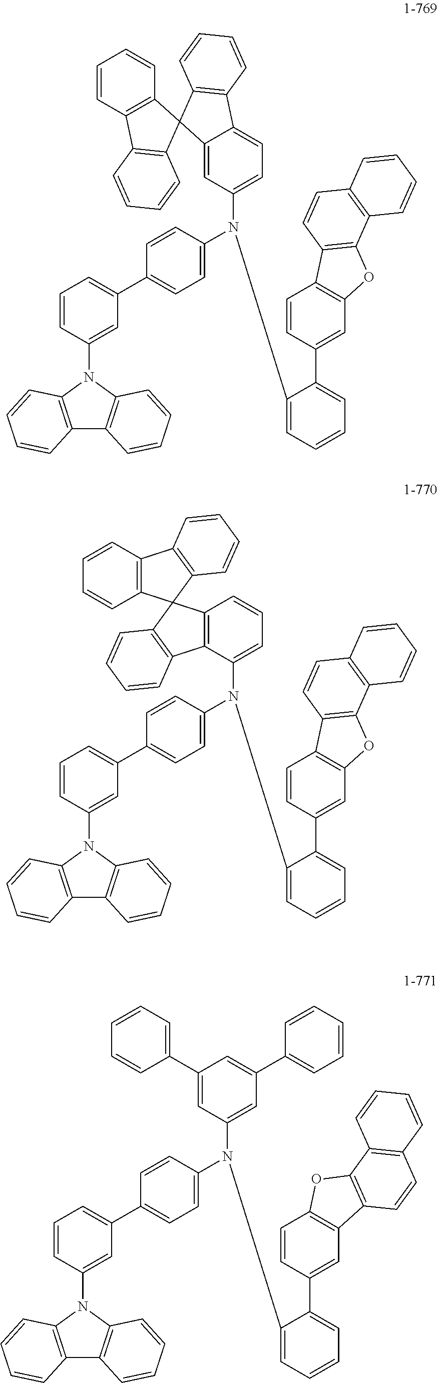 [SMILES: c1cc(-c2ccc(N(c3ccc4c(c3)C3(c5ccccc5-c5ccccc53)c3ccccc3-4)c3ccccc3-c3ccc4c(c3)oc3c5ccccc5ccc43)cc2)cc(-n2c3ccccc3c3ccccc32)c1 ; c1cc(-c2ccc(N(c3ccccc3-c3ccc4c(c3)oc3c5ccccc5ccc43)c3cccc4c3-c3ccccc3C43c4ccccc4-c4ccccc43)cc2)cc(-n2c3ccccc3c3ccccc32)c1 ; c1ccc(-c2cc(-c3ccccc3)cc(N(c3ccc(-c4cccc(-n5c6ccccc6c6ccccc65)c4)cc3)c3ccccc3-c3ccc4oc5c6ccccc6ccc5c4c3)c2)cc1]

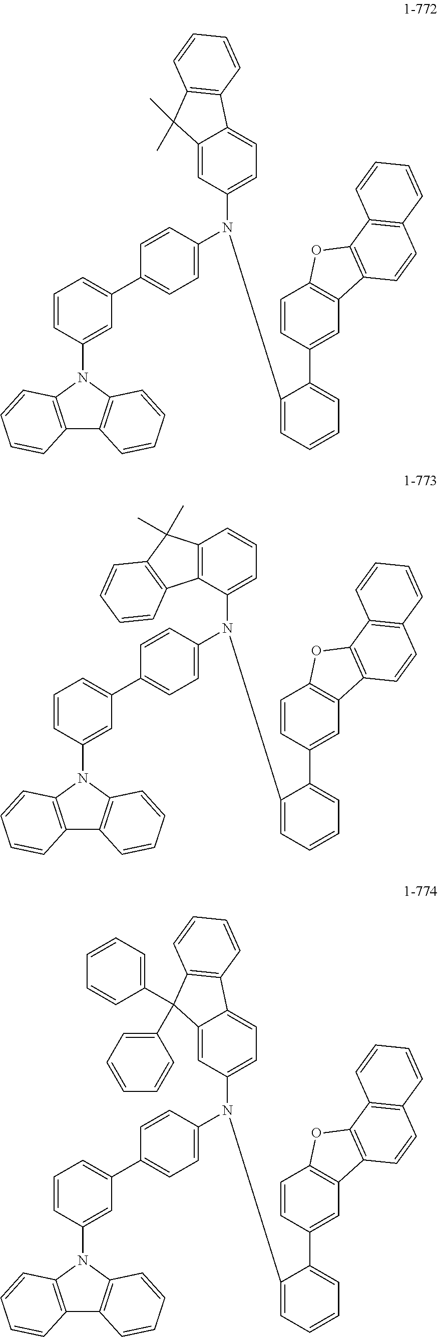 [SMILES: CC1(C)c2ccccc2-c2ccc(N(c3ccc(-c4cccc(-n5c6ccccc6c6ccccc65)c4)cc3)c3ccccc3-c3ccc4oc5c6ccccc6ccc5c4c3)cc21 ; CC1(C)c2ccccc2-c2c(N(c3ccc(-c4cccc(-n5c6ccccc6c6ccccc65)c4)cc3)c3ccccc3-c3ccc4oc5c6ccccc6ccc5c4c3)cccc21 ; c1ccc(C2(c3ccccc3)c3ccccc3-c3ccc(N(c4ccc(-c5cccc(-n6c7ccccc7c7ccccc76)c5)cc4)c4ccccc4-c4ccc5oc6c7ccccc7ccc6c5c4)cc32)cc1]

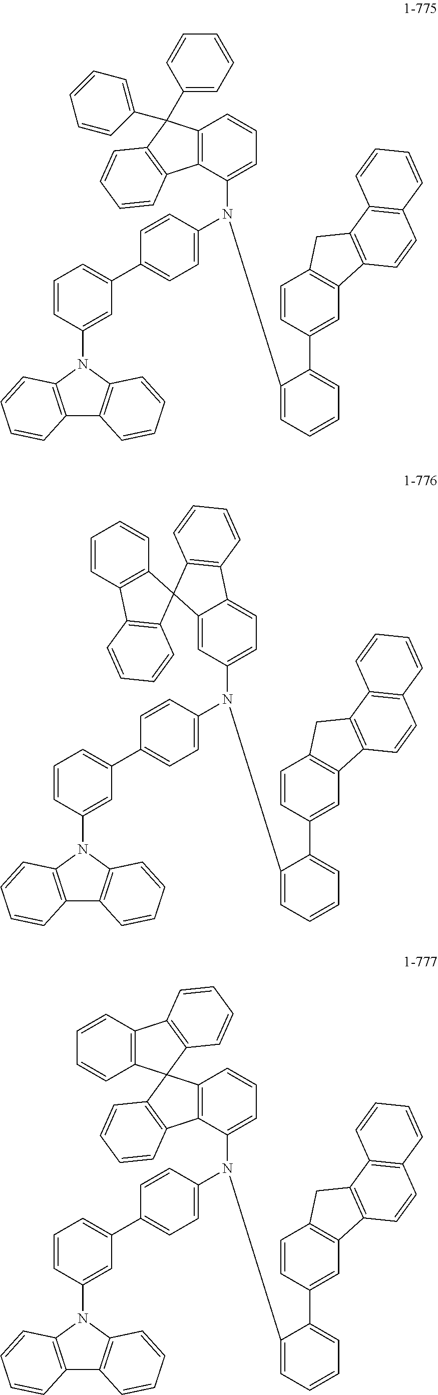 [SMILES: c1ccc(C2(c3ccccc3)c3ccccc3-c3c(N(c4ccc(-c5cccc(-n6c7ccccc7c7ccccc76)c5)cc4)c4ccccc4-c4ccc5c(c4)-c4ccc6ccccc6c4C5)cccc32)cc1 ; c1cc(-c2ccc(N(c3ccc4c(c3)C3(c5ccccc5-c5ccccc53)c3ccccc3-4)c3ccccc3-c3ccc4c(c3)-c3ccc5ccccc5c3C4)cc2)cc(-n2c3ccccc3c3ccccc32)c1 ; c1cc(-c2ccc(N(c3ccccc3-c3ccc4c(c3)-c3ccc5ccccc5c3C4)c3cccc4c3-c3ccccc3C43c4ccccc4-c4ccccc43)cc2)cc(-n2c3ccccc3c3ccccc32)c1]

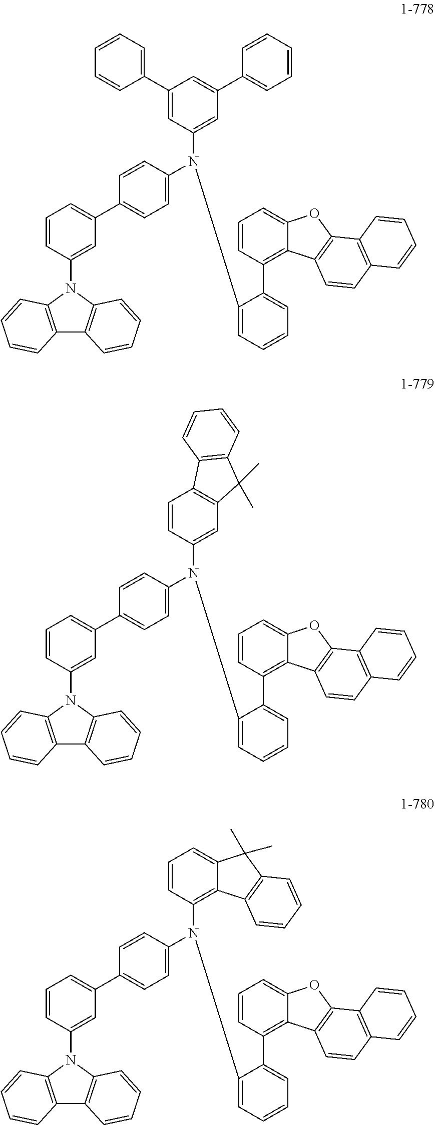 [SMILES: c1ccc(-c2cc(-c3ccccc3)cc(N(c3ccc(-c4cccc(-n5c6ccccc6c6ccccc65)c4)cc3)c3ccccc3-c3cccc4oc5c6ccccc6ccc5c34)c2)cc1 ; CC1(C)c2ccccc2-c2ccc(N(c3ccc(-c4cccc(-n5c6ccccc6c6ccccc65)c4)cc3)c3ccccc3-c3cccc4oc5c6ccccc6ccc5c34)cc21 ; CC1(C)c2ccccc2-c2c(N(c3ccc(-c4cccc(-n5c6ccccc6c6ccccc65)c4)cc3)c3ccccc3-c3cccc4oc5c6ccccc6ccc5c34)cccc21]

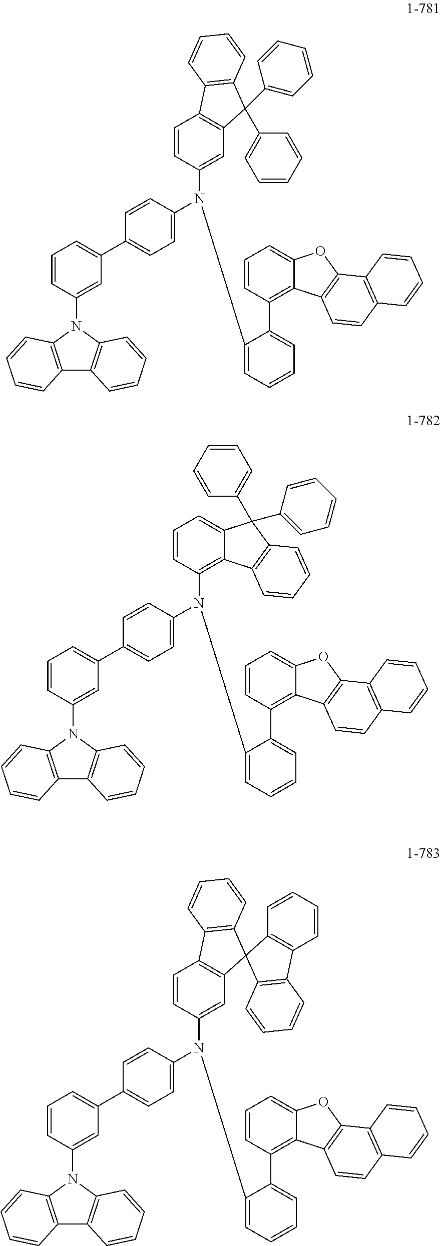 [SMILES: c1ccc(C2(c3ccccc3)c3ccccc3-c3ccc(N(c4ccc(-c5cccc(-n6c7ccccc7c7ccccc76)c5)cc4)c4ccccc4-c4cccc5oc6c7ccccc7ccc6c45)cc32)cc1 ; c1ccc(C2(c3ccccc3)c3ccccc3-c3c(N(c4ccc(-c5cccc(-n6c7ccccc7c7ccccc76)c5)cc4)c4ccccc4-c4cccc5oc6c7ccccc7ccc6c45)cccc32)cc1 ; c1cc(-c2ccc(N(c3ccc4c(c3)C3(c5ccccc5-c5ccccc53)c3ccccc3-4)c3ccccc3-c3cccc4oc5c6ccccc6ccc5c34)cc2)cc(-n2c3ccccc3c3ccccc32)c1]

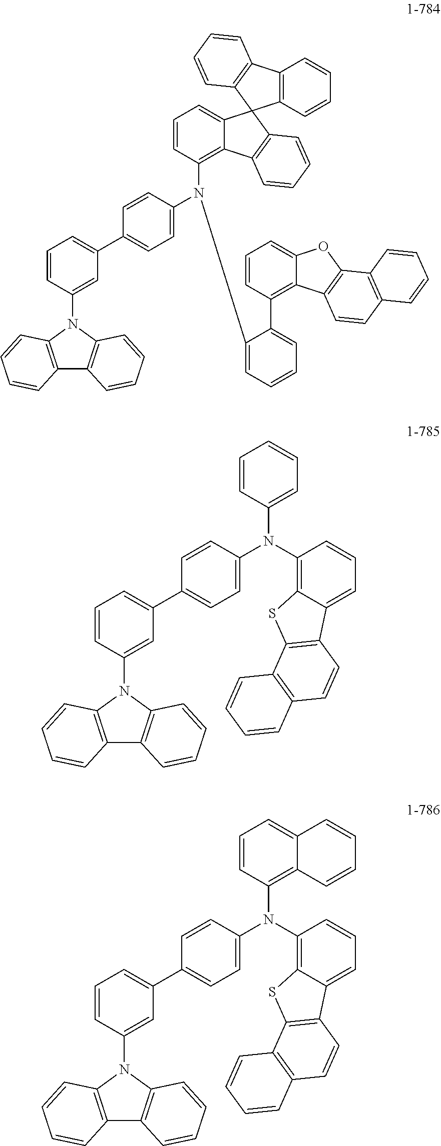 [SMILES: c1cc(-c2ccc(N(c3ccccc3-c3cccc4oc5c6ccccc6ccc5c34)c3cccc4c3-c3ccccc3C43c4ccccc4-c4ccccc43)cc2)cc(-n2c3ccccc3c3ccccc32)c1 ; c1ccc(N(c2ccc(-c3cccc(-n4c5ccccc5c5ccccc54)c3)cc2)c2cccc3c2sc2c4ccccc4ccc32)cc1 ; c1cc(-c2ccc(N(c3cccc4ccccc34)c3cccc4c3sc3c5ccccc5ccc43)cc2)cc(-n2c3ccccc3c3ccccc32)c1]

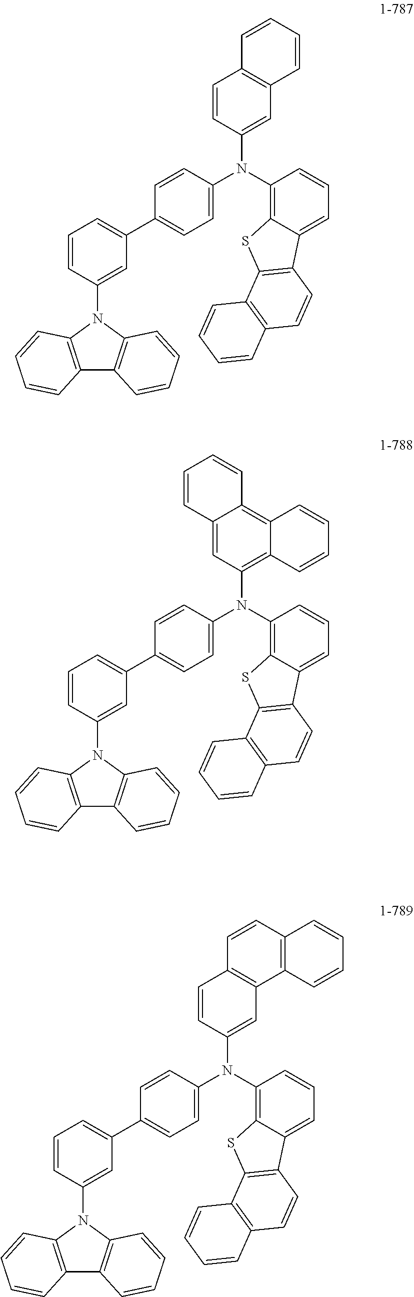 [SMILES: c1cc(-c2ccc(N(c3ccc4ccccc4c3)c3cccc4c3sc3c5ccccc5ccc43)cc2)cc(-n2c3ccccc3c3ccccc32)c1 ; c1cc(-c2ccc(N(c3cc4ccccc4c4ccccc34)c3cccc4c3sc3c5ccccc5ccc43)cc2)cc(-n2c3ccccc3c3ccccc32)c1 ; c1cc(-c2ccc(N(c3ccc4ccc5ccccc5c4c3)c3cccc4c3sc3c5ccccc5ccc43)cc2)cc(-n2c3ccccc3c3ccccc32)c1]

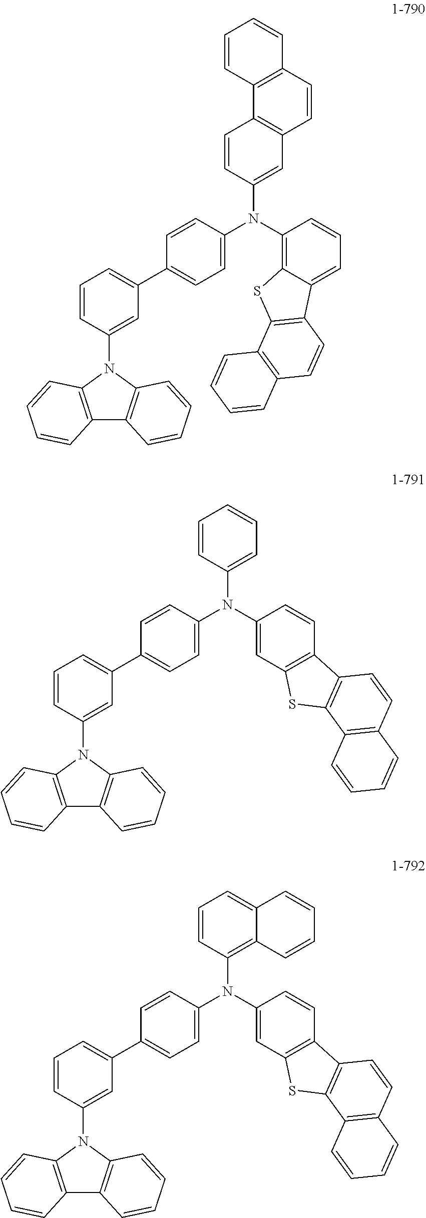 [SMILES: c1cc(-c2ccc(N(c3ccc4c(ccc5ccccc54)c3)c3cccc4c3sc3c5ccccc5ccc43)cc2)cc(-n2c3ccccc3c3ccccc32)c1 ; c1ccc(N(c2ccc(-c3cccc(-n4c5ccccc5c5ccccc54)c3)cc2)c2ccc3c(c2)sc2c4ccccc4ccc32)cc1 ; c1cc(-c2ccc(N(c3ccc4c(c3)sc3c5ccccc5ccc43)c3cccc4ccccc34)cc2)cc(-n2c3ccccc3c3ccccc32)c1]

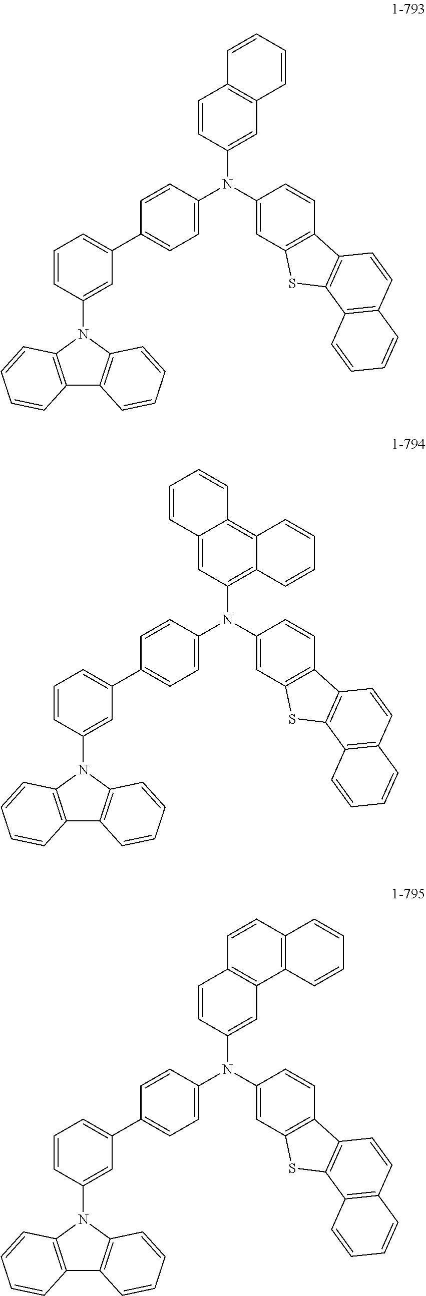 [SMILES: c1cc(-c2ccc(N(c3ccc4ccccc4c3)c3ccc4c(c3)sc3c5ccccc5ccc43)cc2)cc(-n2c3ccccc3c3ccccc32)c1 ; c1cc(-c2ccc(N(c3ccc4c(c3)sc3c5ccccc5ccc43)c3cc4ccccc4c4ccccc34)cc2)cc(-n2c3ccccc3c3ccccc32)c1 ; c1cc(-c2ccc(N(c3ccc4c(c3)sc3c5ccccc5ccc43)c3ccc4ccc5ccccc5c4c3)cc2)cc(-n2c3ccccc3c3ccccc32)c1]

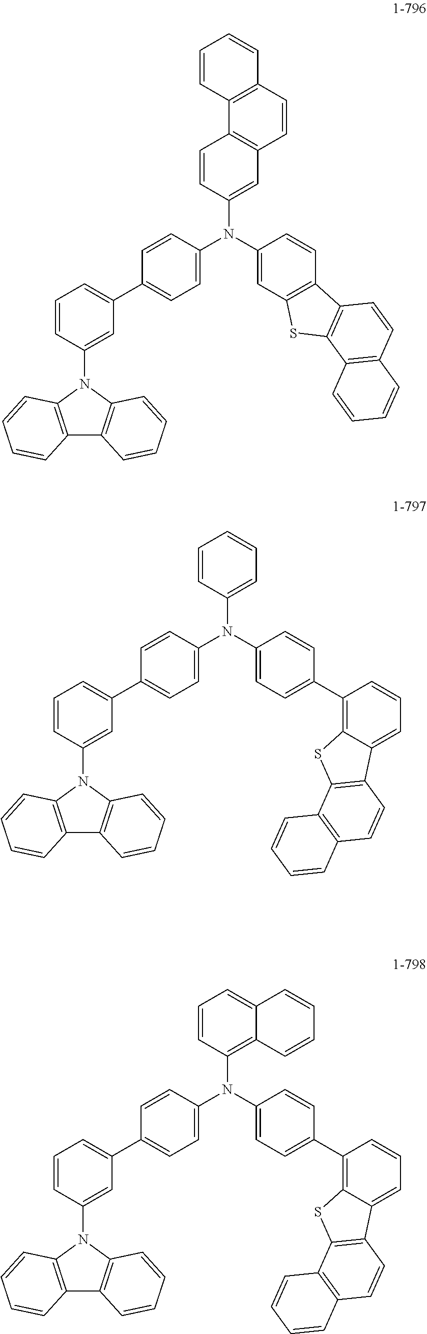 [SMILES: c1cc(-c2ccc(N(c3ccc4c(ccc5ccccc54)c3)c3ccc4c(c3)sc3c5ccccc5ccc43)cc2)cc(-n2c3ccccc3c3ccccc32)c1 ; c1ccc(N(c2ccc(-c3cccc(-n4c5ccccc5c5ccccc54)c3)cc2)c2ccc(-c3cccc4c3sc3c5ccccc5ccc43)cc2)cc1 ; c1cc(-c2ccc(N(c3ccc(-c4cccc5c4sc4c6ccccc6ccc54)cc3)c3cccc4ccccc34)cc2)cc(-n2c3ccccc3c3ccccc32)c1]

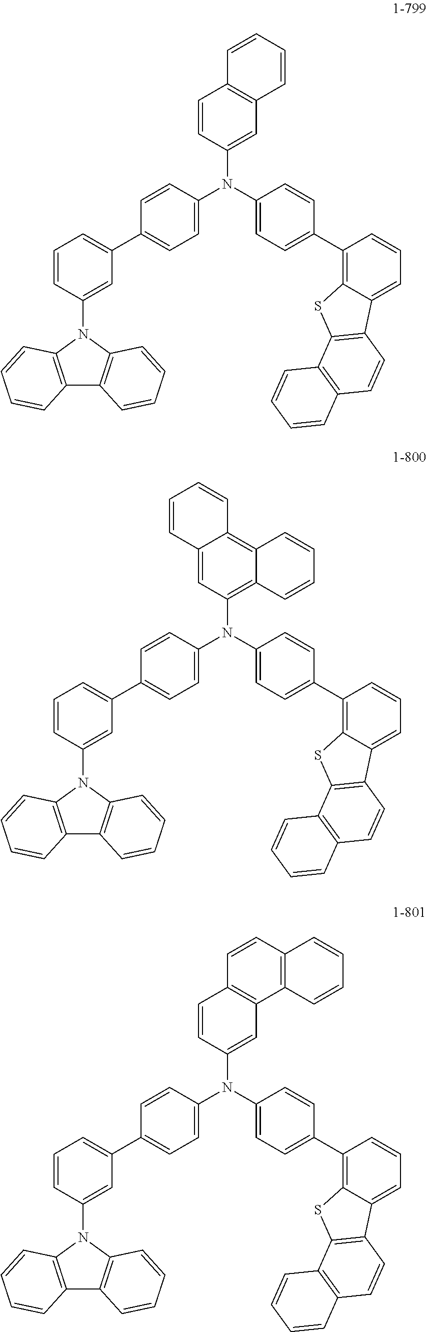 [SMILES: c1cc(-c2ccc(N(c3ccc(-c4cccc5c4sc4c6ccccc6ccc54)cc3)c3ccc4ccccc4c3)cc2)cc(-n2c3ccccc3c3ccccc32)c1 ; c1cc(-c2ccc(N(c3ccc(-c4cccc5c4sc4c6ccccc6ccc54)cc3)c3cc4ccccc4c4ccccc34)cc2)cc(-n2c3ccccc3c3ccccc32)c1 ; c1cc(-c2ccc(N(c3ccc(-c4cccc5c4sc4c6ccccc6ccc54)cc3)c3ccc4ccc5ccccc5c4c3)cc2)cc(-n2c3ccccc3c3ccccc32)c1]

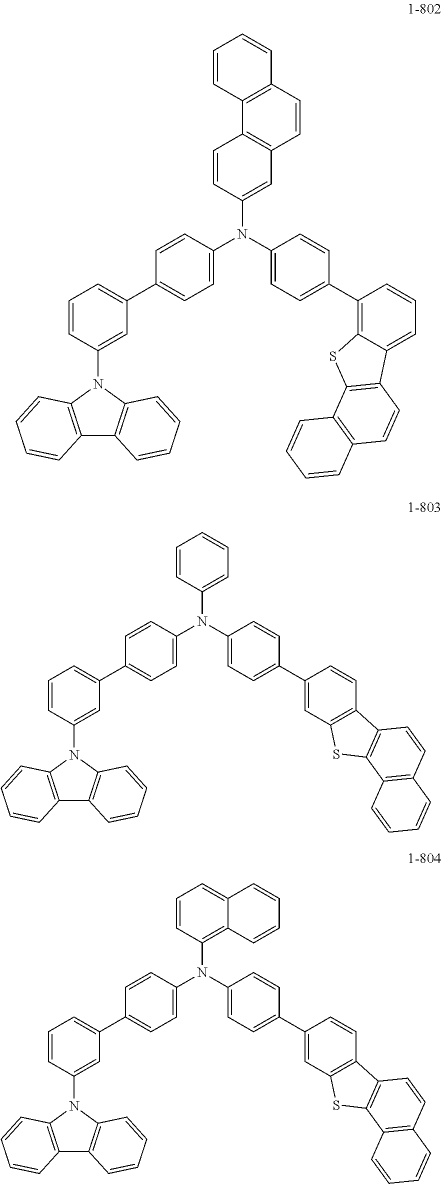 [SMILES: c1cc(-c2ccc(N(c3ccc(-c4cccc5c4sc4c6ccccc6ccc54)cc3)c3ccc4c(ccc5ccccc54)c3)cc2)cc(-n2c3ccccc3c3ccccc32)c1 ; c1ccc(N(c2ccc(-c3cccc(-n4c5ccccc5c5ccccc54)c3)cc2)c2ccc(-c3ccc4c(c3)sc3c5ccccc5ccc43)cc2)cc1 ; c1cc(-c2ccc(N(c3ccc(-c4ccc5c(c4)sc4c6ccccc6ccc54)cc3)c3cccc4ccccc34)cc2)cc(-n2c3ccccc3c3ccccc32)c1]

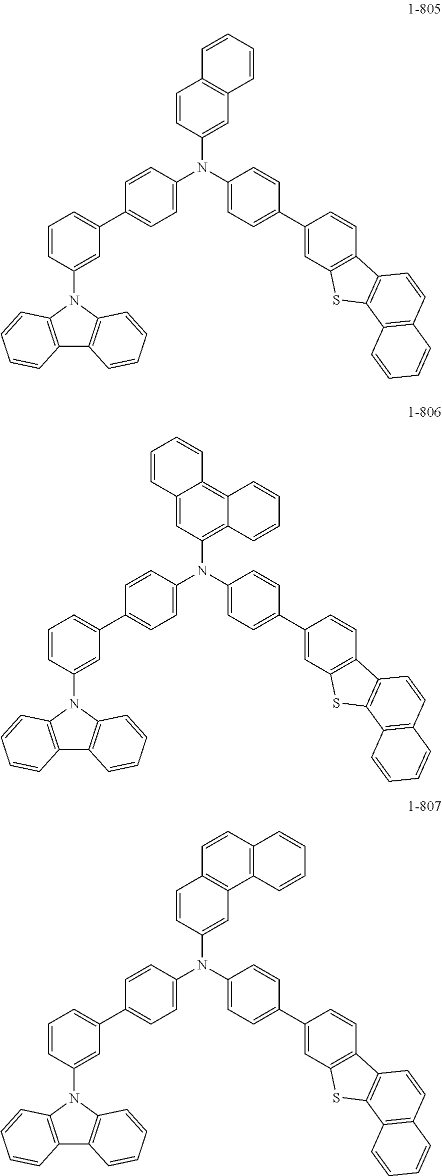 [SMILES: c1cc(-c2ccc(N(c3ccc(-c4ccc5c(c4)sc4c6ccccc6ccc54)cc3)c3ccc4ccccc4c3)cc2)cc(-n2c3ccccc3c3ccccc32)c1 ; c1cc(-c2ccc(N(c3ccc(-c4ccc5c(c4)sc4c6ccccc6ccc54)cc3)c3cc4ccccc4c4ccccc34)cc2)cc(-n2c3ccccc3c3ccccc32)c1 ; c1cc(-c2ccc(N(c3ccc(-c4ccc5c(c4)sc4c6ccccc6ccc54)cc3)c3ccc4ccc5ccccc5c4c3)cc2)cc(-n2c3ccccc3c3ccccc32)c1]

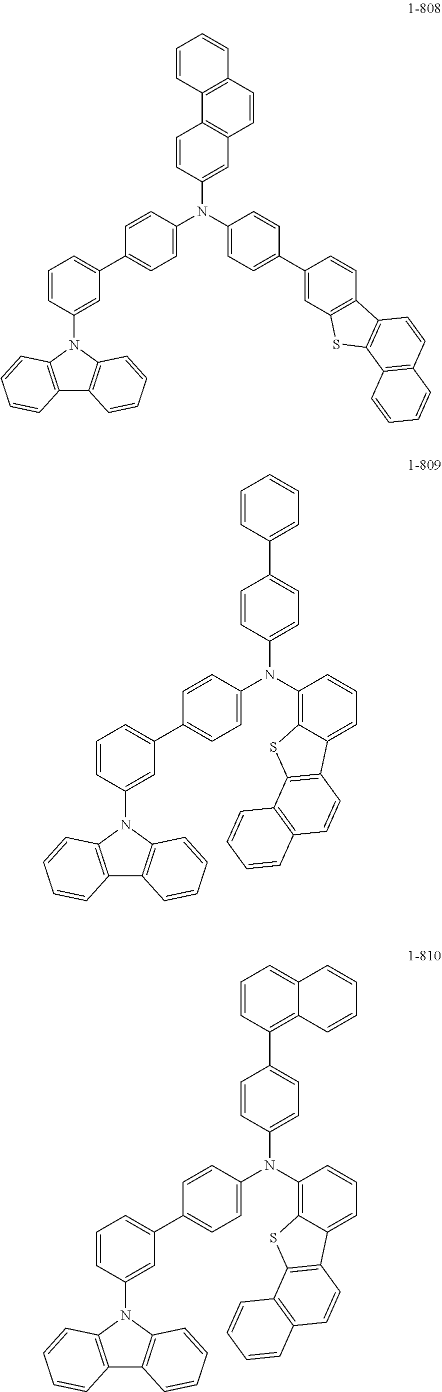 [SMILES: c1cc(-c2ccc(N(c3ccc(-c4ccc5c(c4)sc4c6ccccc6ccc54)cc3)c3ccc4c(ccc5ccccc54)c3)cc2)cc(-n2c3ccccc3c3ccccc32)c1 ; c1ccc(-c2ccc(N(c3ccc(-c4cccc(-n5c6ccccc6c6ccccc65)c4)cc3)c3cccc4c3sc3c5ccccc5ccc43)cc2)cc1 ; c1cc(-c2ccc(N(c3ccc(-c4cccc5ccccc45)cc3)c3cccc4c3sc3c5ccccc5ccc43)cc2)cc(-n2c3ccccc3c3ccccc32)c1]

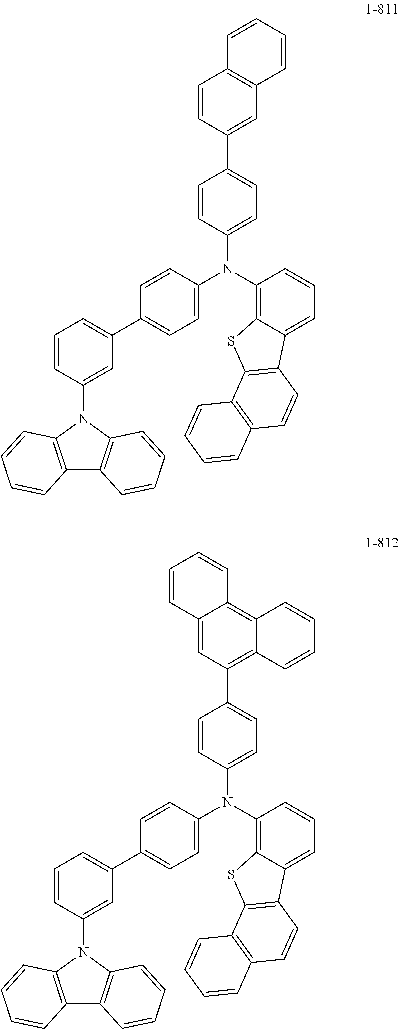 [SMILES: c1cc(-c2ccc(N(c3ccc(-c4ccc5ccccc5c4)cc3)c3cccc4c3sc3c5ccccc5ccc43)cc2)cc(-n2c3ccccc3c3ccccc32)c1 ; c1cc(-c2ccc(N(c3ccc(-c4cc5ccccc5c5ccccc45)cc3)c3cccc4c3sc3c5ccccc5ccc43)cc2)cc(-n2c3ccccc3c3ccccc32)c1]

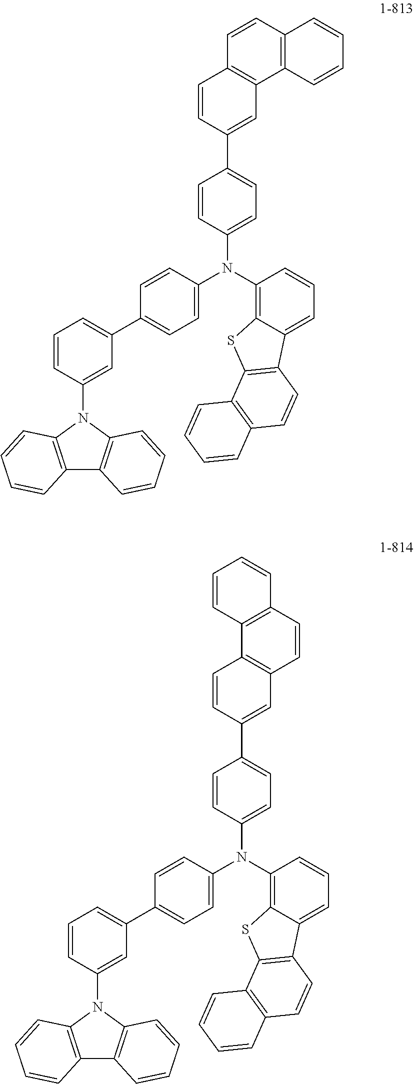 [SMILES: c1cc(-c2ccc(N(c3ccc(-c4ccc5ccc6ccccc6c5c4)cc3)c3cccc4c3sc3c5ccccc5ccc43)cc2)cc(-n2c3ccccc3c3ccccc32)c1 ; c1cc(-c2ccc(N(c3ccc(-c4ccc5c(ccc6ccccc65)c4)cc3)c3cccc4c3sc3c5ccccc5ccc43)cc2)cc(-n2c3ccccc3c3ccccc32)c1]

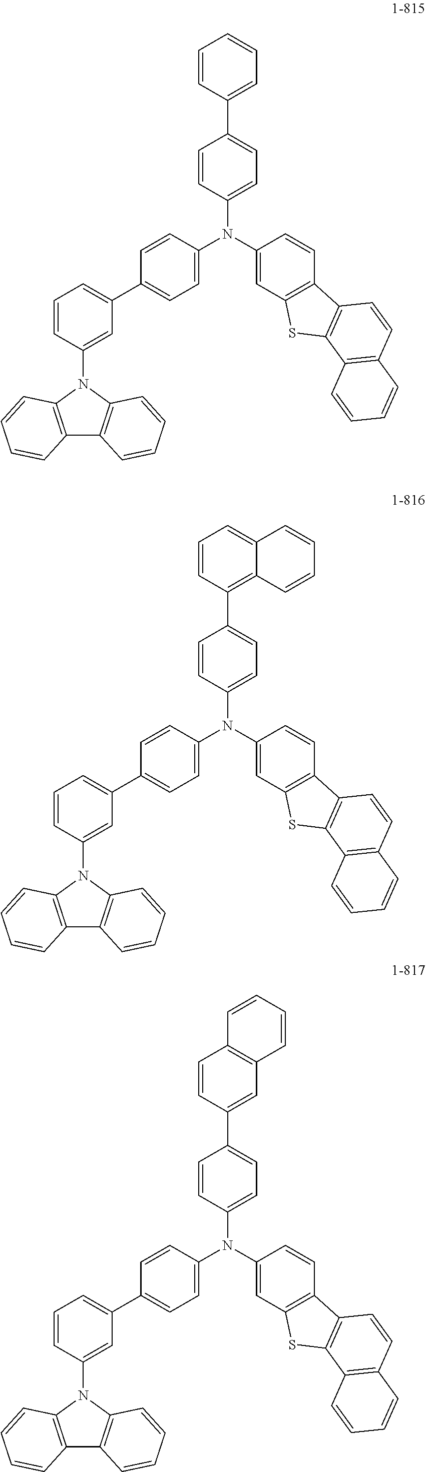 [SMILES: c1ccc(-c2ccc(N(c3ccc(-c4cccc(-n5c6ccccc6c6ccccc65)c4)cc3)c3ccc4c(c3)sc3c5ccccc5ccc43)cc2)cc1 ; c1cc(-c2ccc(N(c3ccc(-c4cccc5ccccc45)cc3)c3ccc4c(c3)sc3c5ccccc5ccc43)cc2)cc(-n2c3ccccc3c3ccccc32)c1 ; c1cc(-c2ccc(N(c3ccc(-c4ccc5ccccc5c4)cc3)c3ccc4c(c3)sc3c5ccccc5ccc43)cc2)cc(-n2c3ccccc3c3ccccc32)c1]

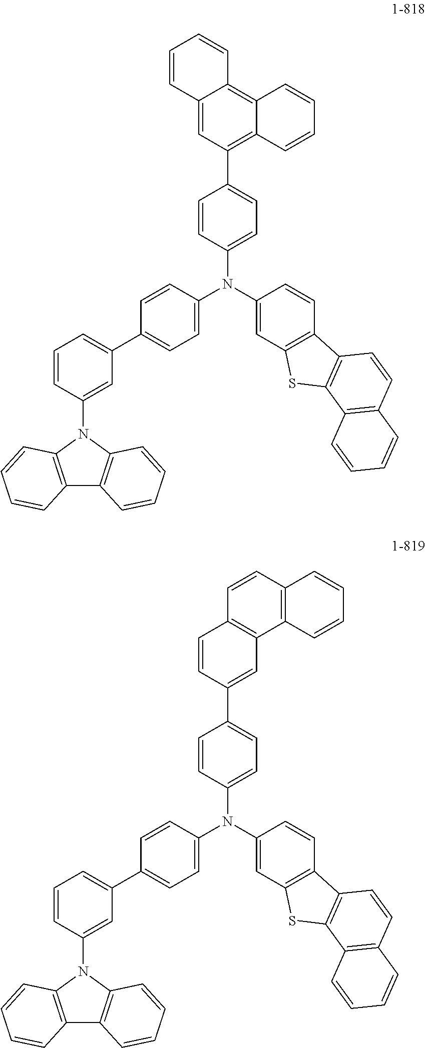 [SMILES: c1cc(-c2ccc(N(c3ccc(-c4cc5ccccc5c5ccccc45)cc3)c3ccc4c(c3)sc3c5ccccc5ccc43)cc2)cc(-n2c3ccccc3c3ccccc32)c1 ; c1cc(-c2ccc(N(c3ccc(-c4ccc5ccc6ccccc6c5c4)cc3)c3ccc4c(c3)sc3c5ccccc5ccc43)cc2)cc(-n2c3ccccc3c3ccccc32)c1]

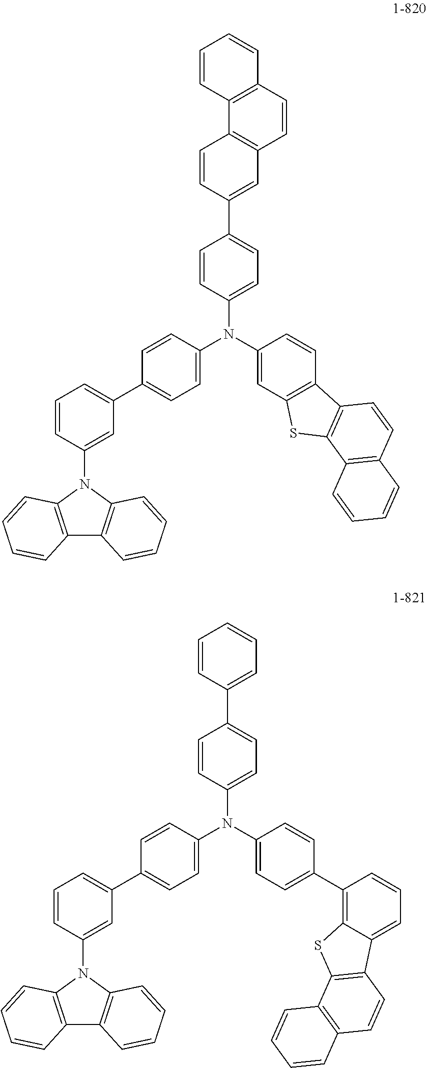 [SMILES: c1cc(-c2ccc(N(c3ccc(-c4ccc5c(ccc6ccccc65)c4)cc3)c3ccc4c(c3)sc3c5ccccc5ccc43)cc2)cc(-n2c3ccccc3c3ccccc32)c1 ; c1ccc(-c2ccc(N(c3ccc(-c4cccc(-n5c6ccccc6c6ccccc65)c4)cc3)c3ccc(-c4cccc5c4sc4c6ccccc6ccc54)cc3)cc2)cc1]

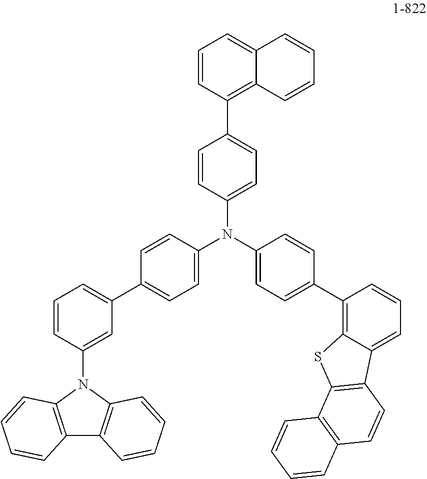 [SMILES: c1cc(-c2ccc(N(c3ccc(-c4cccc5ccccc45)cc3)c3ccc(-c4cccc5c4sc4c6ccccc6ccc54)cc3)cc2)cc(-n2c3ccccc3c3ccccc32)c1]

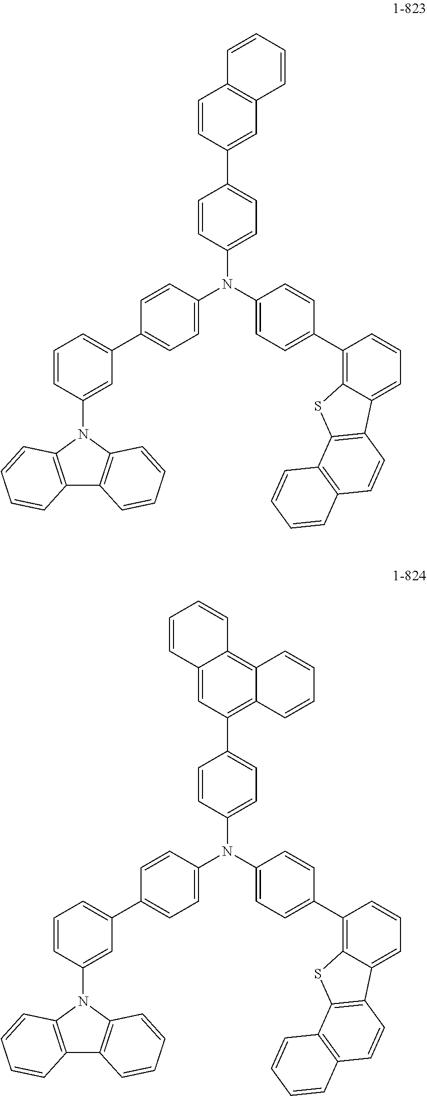 [SMILES: c1cc(-c2ccc(N(c3ccc(-c4ccc5ccccc5c4)cc3)c3ccc(-c4cccc5c4sc4c6ccccc6ccc54)cc3)cc2)cc(-n2c3ccccc3c3ccccc32)c1 ; c1cc(-c2ccc(N(c3ccc(-c4cc5ccccc5c5ccccc45)cc3)c3ccc(-c4cccc5c4sc4c6ccccc6ccc54)cc3)cc2)cc(-n2c3ccccc3c3ccccc32)c1]

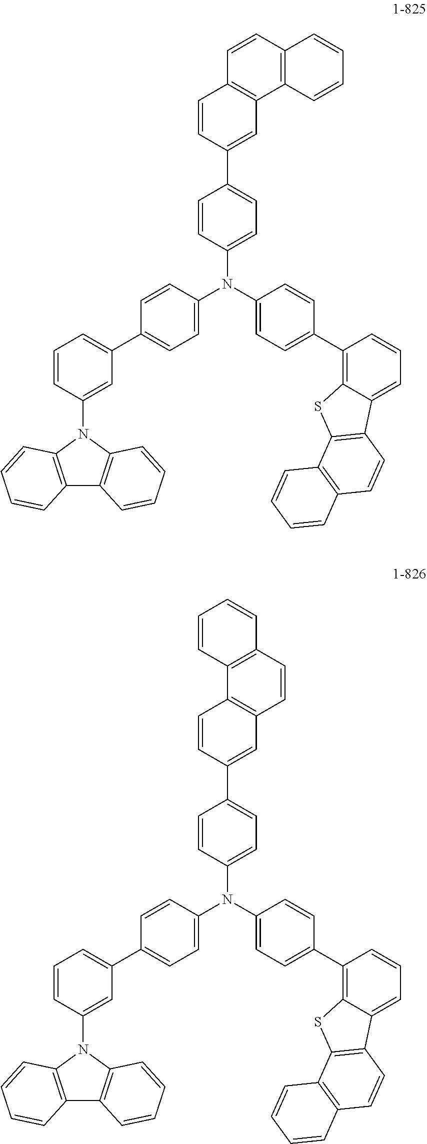 [SMILES: c1cc(-c2ccc(N(c3ccc(-c4ccc5ccc6ccccc6c5c4)cc3)c3ccc(-c4cccc5c4sc4c6ccccc6ccc54)cc3)cc2)cc(-n2c3ccccc3c3ccccc32)c1 ; c1cc(-c2ccc(N(c3ccc(-c4ccc5c(ccc6ccccc65)c4)cc3)c3ccc(-c4cccc5c4sc4c6ccccc6ccc54)cc3)cc2)cc(-n2c3ccccc3c3ccccc32)c1]

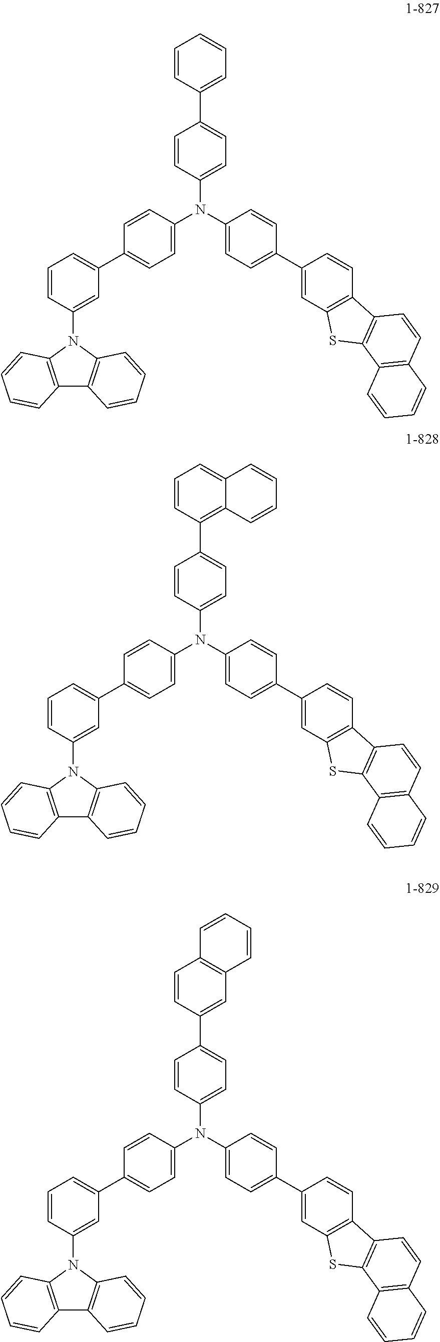 [SMILES: c1ccc(-c2ccc(N(c3ccc(-c4cccc(-n5c6ccccc6c6ccccc65)c4)cc3)c3ccc(-c4ccc5c(c4)sc4c6ccccc6ccc54)cc3)cc2)cc1 ; c1cc(-c2ccc(N(c3ccc(-c4ccc5c(c4)sc4c6ccccc6ccc54)cc3)c3ccc(-c4cccc5ccccc45)cc3)cc2)cc(-n2c3ccccc3c3ccccc32)c1 ; c1cc(-c2ccc(N(c3ccc(-c4ccc5ccccc5c4)cc3)c3ccc(-c4ccc5c(c4)sc4c6ccccc6ccc54)cc3)cc2)cc(-n2c3ccccc3c3ccccc32)c1]

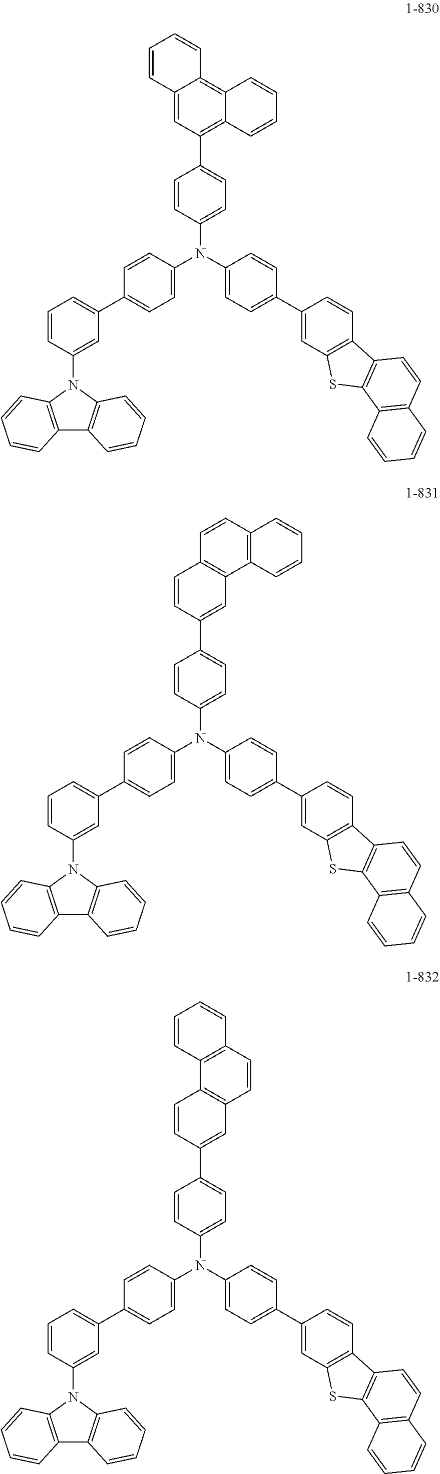 [SMILES: c1cc(-c2ccc(N(c3ccc(-c4ccc5c(c4)sc4c6ccccc6ccc54)cc3)c3ccc(-c4cc5ccccc5c5ccccc45)cc3)cc2)cc(-n2c3ccccc3c3ccccc32)c1 ; c1cc(-c2ccc(N(c3ccc(-c4ccc5c(c4)sc4c6ccccc6ccc54)cc3)c3ccc(-c4ccc5ccc6ccccc6c5c4)cc3)cc2)cc(-n2c3ccccc3c3ccccc32)c1 ; c1cc(-c2ccc(N(c3ccc(-c4ccc5c(ccc6ccccc65)c4)cc3)c3ccc(-c4ccc5c(c4)sc4c6ccccc6ccc54)cc3)cc2)cc(-n2c3ccccc3c3ccccc32)c1]

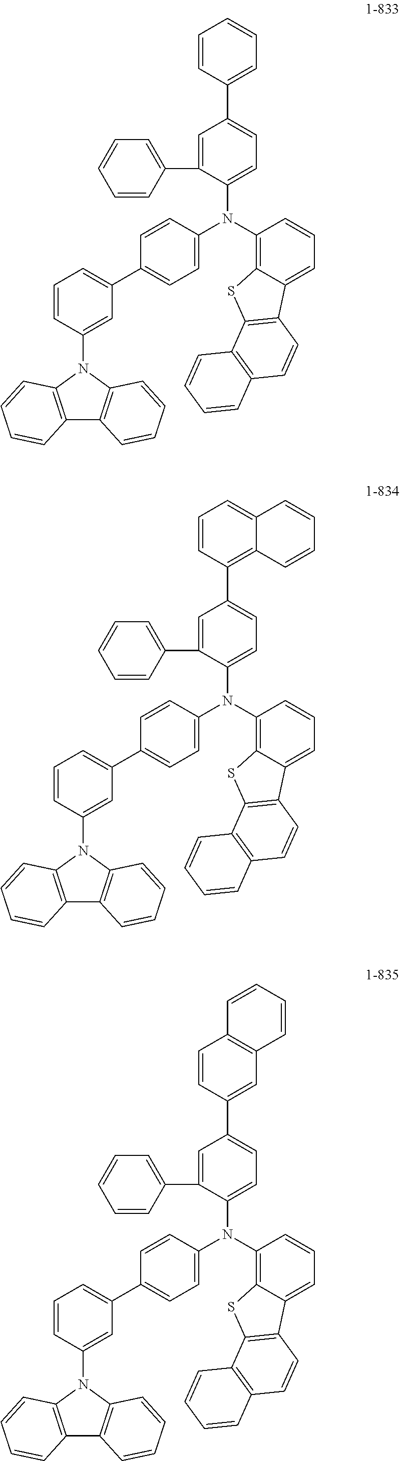 [SMILES: c1ccc(-c2ccc(N(c3ccc(-c4cccc(-n5c6ccccc6c6ccccc65)c4)cc3)c3cccc4c3sc3c5ccccc5ccc43)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2cccc3c2sc2c4ccccc4ccc32)cc1 ; c1ccc(-c2cc(-c3ccc4ccccc4c3)ccc2N(c2ccc(-c3cccc(-n4c5ccccc5c5ccccc54)c3)cc2)c2cccc3c2sc2c4ccccc4ccc32)cc1]

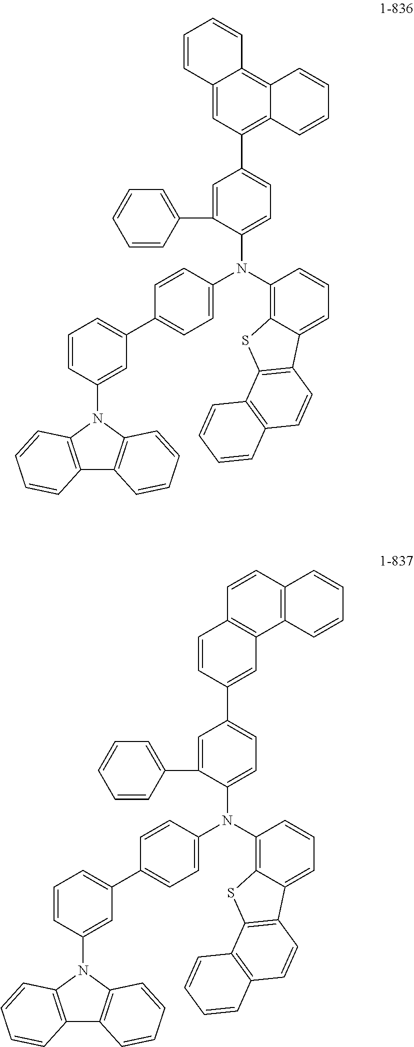 [SMILES: c1ccc(-c2cc(-c3cc4ccccc4c4ccccc34)ccc2N(c2ccc(-c3cccc(-n4c5ccccc5c5ccccc54)c3)cc2)c2cccc3c2sc2c4ccccc4ccc32)cc1 ; c1ccc(-c2cc(-c3ccc4ccc5ccccc5c4c3)ccc2N(c2ccc(-c3cccc(-n4c5ccccc5c5ccccc54)c3)cc2)c2cccc3c2sc2c4ccccc4ccc32)cc1]

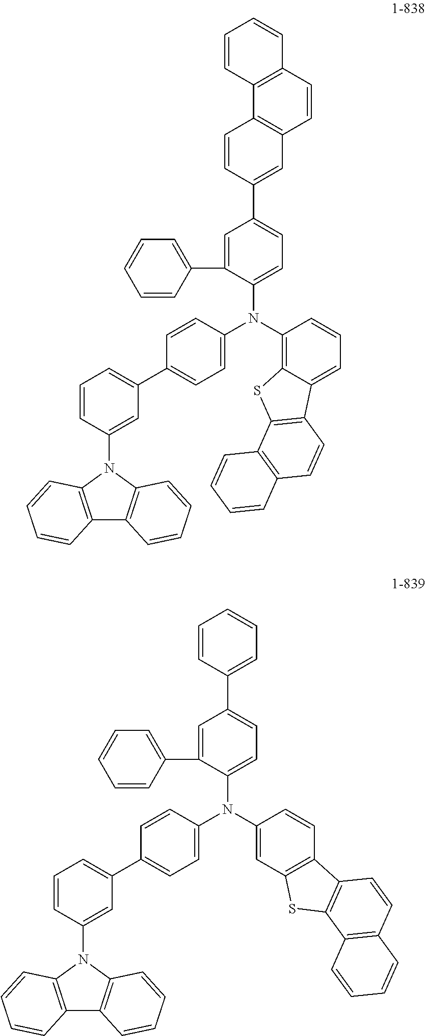 [SMILES: c1ccc(-c2cc(-c3ccc4c(ccc5ccccc54)c3)ccc2N(c2ccc(-c3cccc(-n4c5ccccc5c5ccccc54)c3)cc2)c2cccc3c2sc2c4ccccc4ccc32)cc1 ; c1ccc(-c2ccc(N(c3ccc(-c4cccc(-n5c6ccccc6c6ccccc65)c4)cc3)c3ccc4c(c3)sc3c5ccccc5ccc43)c(-c3ccccc3)c2)cc1]

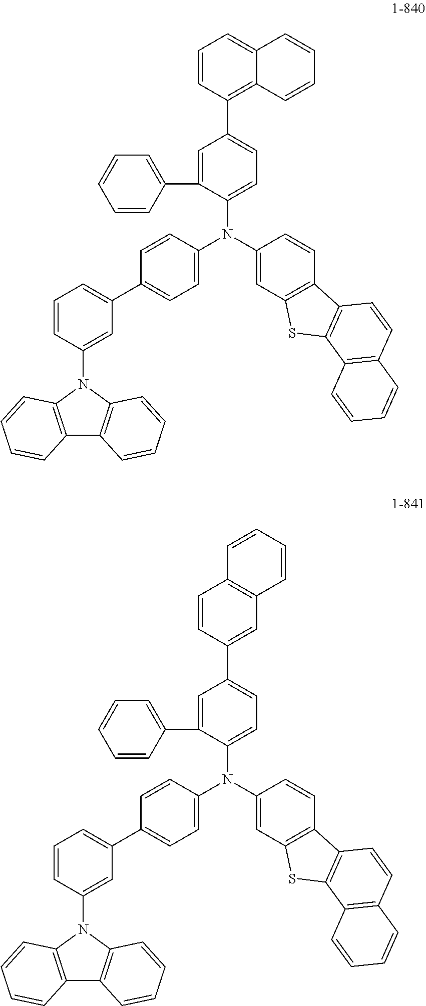 [SMILES: c1ccc(-c2cc(-c3cccc4ccccc34)ccc2N(c2ccc(-c3cccc(-n4c5ccccc5c5ccccc54)c3)cc2)c2ccc3c(c2)sc2c4ccccc4ccc32)cc1 ; c1ccc(-c2cc(-c3ccc4ccccc4c3)ccc2N(c2ccc(-c3cccc(-n4c5ccccc5c5ccccc54)c3)cc2)c2ccc3c(c2)sc2c4ccccc4ccc32)cc1]

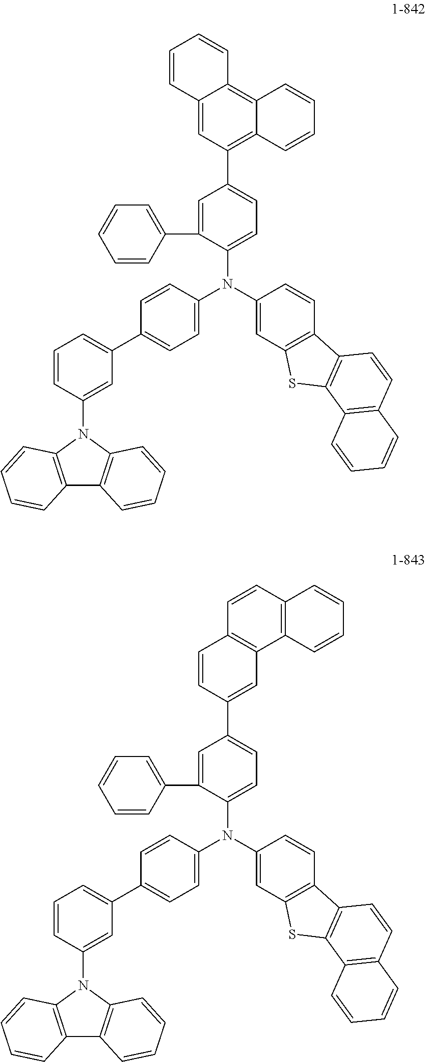 [SMILES: c1ccc(-c2cc(-c3cc4ccccc4c4ccccc34)ccc2N(c2ccc(-c3cccc(-n4c5ccccc5c5ccccc54)c3)cc2)c2ccc3c(c2)sc2c4ccccc4ccc32)cc1 ; c1ccc(-c2cc(-c3ccc4ccc5ccccc5c4c3)ccc2N(c2ccc(-c3cccc(-n4c5ccccc5c5ccccc54)c3)cc2)c2ccc3c(c2)sc2c4ccccc4ccc32)cc1]

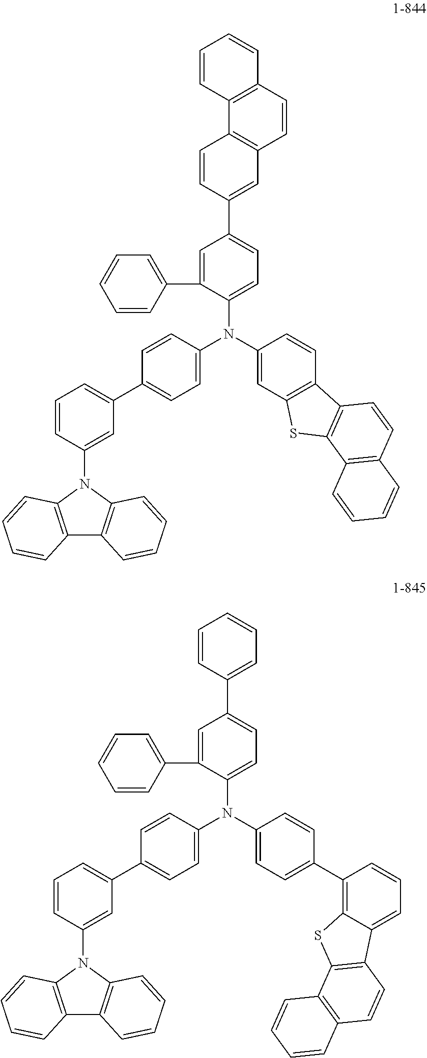 [SMILES: c1ccc(-c2cc(-c3ccc4c(ccc5ccccc54)c3)ccc2N(c2ccc(-c3cccc(-n4c5ccccc5c5ccccc54)c3)cc2)c2ccc3c(c2)sc2c4ccccc4ccc32)cc1 ; c1ccc(-c2ccc(N(c3ccc(-c4cccc(-n5c6ccccc6c6ccccc65)c4)cc3)c3ccc(-c4cccc5c4sc4c6ccccc6ccc54)cc3)c(-c3ccccc3)c2)cc1]

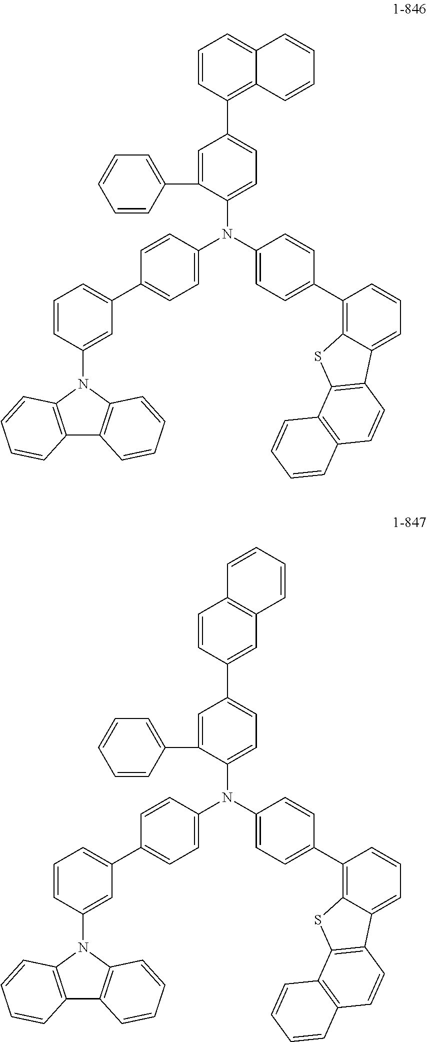 [SMILES: c1ccc(-c2cc(-c3cccc4ccccc34)ccc2N(c2ccc(-c3cccc(-n4c5ccccc5c5ccccc54)c3)cc2)c2ccc(-c3cccc4c3sc3c5ccccc5ccc43)cc2)cc1 ; c1ccc(-c2cc(-c3ccc4ccccc4c3)ccc2N(c2ccc(-c3cccc(-n4c5ccccc5c5ccccc54)c3)cc2)c2ccc(-c3cccc4c3sc3c5ccccc5ccc43)cc2)cc1]

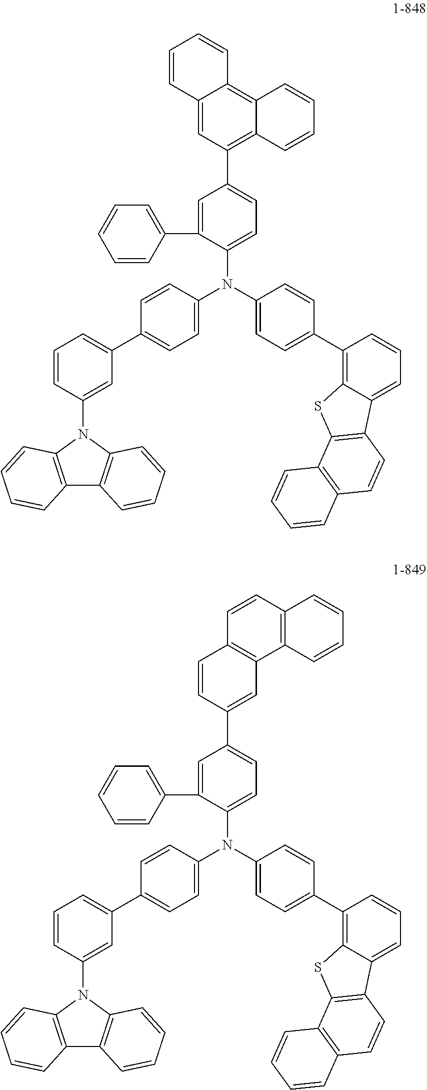 [SMILES: c1ccc(-c2cc(-c3cc4ccccc4c4ccccc34)ccc2N(c2ccc(-c3cccc(-n4c5ccccc5c5ccccc54)c3)cc2)c2ccc(-c3cccc4c3sc3c5ccccc5ccc43)cc2)cc1 ; c1ccc(-c2cc(-c3ccc4ccc5ccccc5c4c3)ccc2N(c2ccc(-c3cccc(-n4c5ccccc5c5ccccc54)c3)cc2)c2ccc(-c3cccc4c3sc3c5ccccc5ccc43)cc2)cc1]

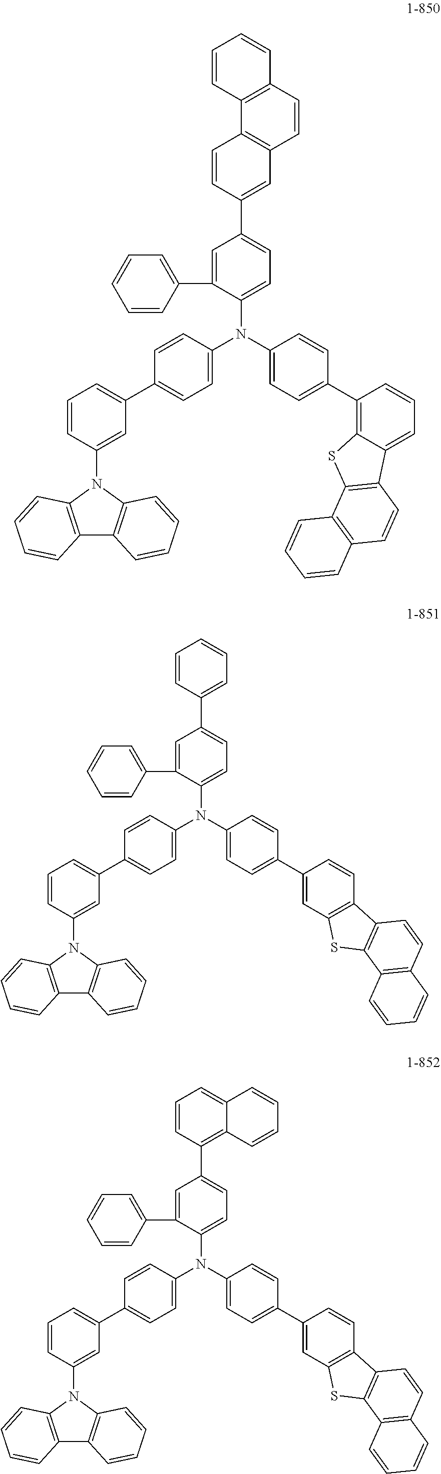 [SMILES: c1ccc(-c2cc(-c3ccc4c(ccc5ccccc54)c3)ccc2N(c2ccc(-c3cccc(-n4c5ccccc5c5ccccc54)c3)cc2)c2ccc(-c3cccc4c3sc3c5ccccc5ccc43)cc2)cc1 ; c1ccc(-c2ccc(N(c3ccc(-c4cccc(-n5c6ccccc6c6ccccc65)c4)cc3)c3ccc(-c4ccc5c(c4)sc4c6ccccc6ccc54)cc3)c(-c3ccccc3)c2)cc1 ; c1ccc(-c2cc(-c3cccc4ccccc34)ccc2N(c2ccc(-c3cccc(-n4c5ccccc5c5ccccc54)c3)cc2)c2ccc(-c3ccc4c(c3)sc3c5ccccc5ccc43)cc2)cc1]

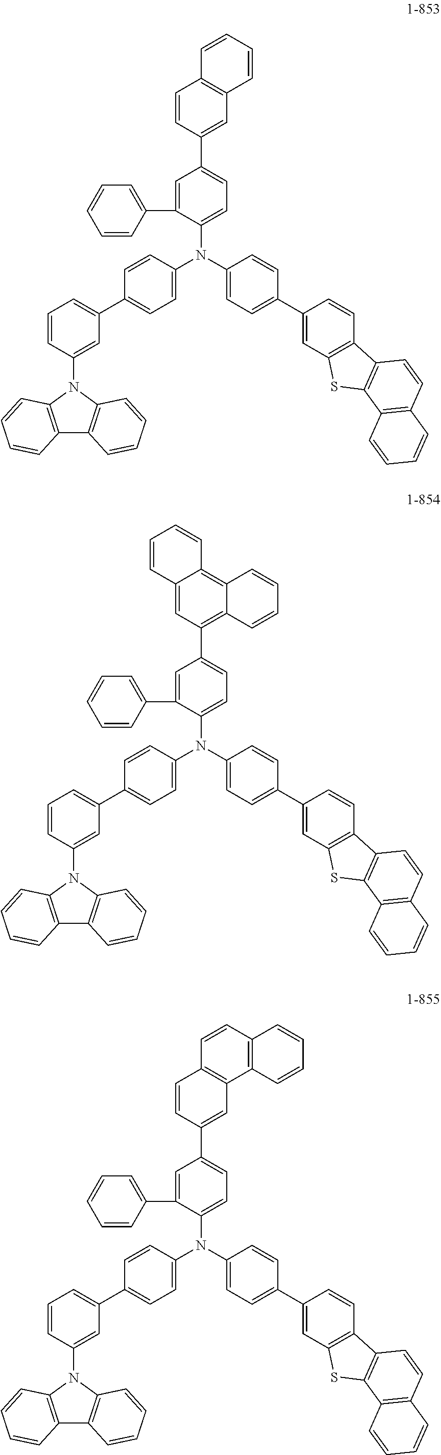 [SMILES: c1ccc(-c2cc(-c3ccc4ccccc4c3)ccc2N(c2ccc(-c3cccc(-n4c5ccccc5c5ccccc54)c3)cc2)c2ccc(-c3ccc4c(c3)sc3c5ccccc5ccc43)cc2)cc1 ; c1ccc(-c2cc(-c3cc4ccccc4c4ccccc34)ccc2N(c2ccc(-c3cccc(-n4c5ccccc5c5ccccc54)c3)cc2)c2ccc(-c3ccc4c(c3)sc3c5ccccc5ccc43)cc2)cc1 ; c1ccc(-c2cc(-c3ccc4ccc5ccccc5c4c3)ccc2N(c2ccc(-c3cccc(-n4c5ccccc5c5ccccc54)c3)cc2)c2ccc(-c3ccc4c(c3)sc3c5ccccc5ccc43)cc2)cc1]

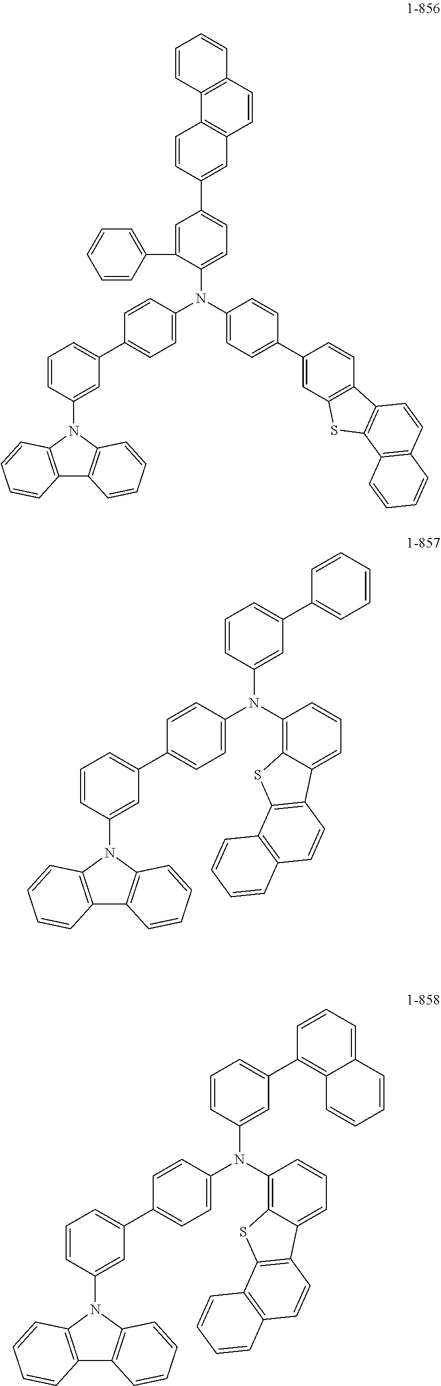 [SMILES: c1ccc(-c2cc(-c3ccc4c(ccc5ccccc54)c3)ccc2N(c2ccc(-c3cccc(-n4c5ccccc5c5ccccc54)c3)cc2)c2ccc(-c3ccc4c(c3)sc3c5ccccc5ccc43)cc2)cc1 ; c1ccc(-c2cccc(N(c3ccc(-c4cccc(-n5c6ccccc6c6ccccc65)c4)cc3)c3cccc4c3sc3c5ccccc5ccc43)c2)cc1 ; c1cc(-c2cccc3ccccc23)cc(N(c2ccc(-c3cccc(-n4c5ccccc5c5ccccc54)c3)cc2)c2cccc3c2sc2c4ccccc4ccc32)c1]

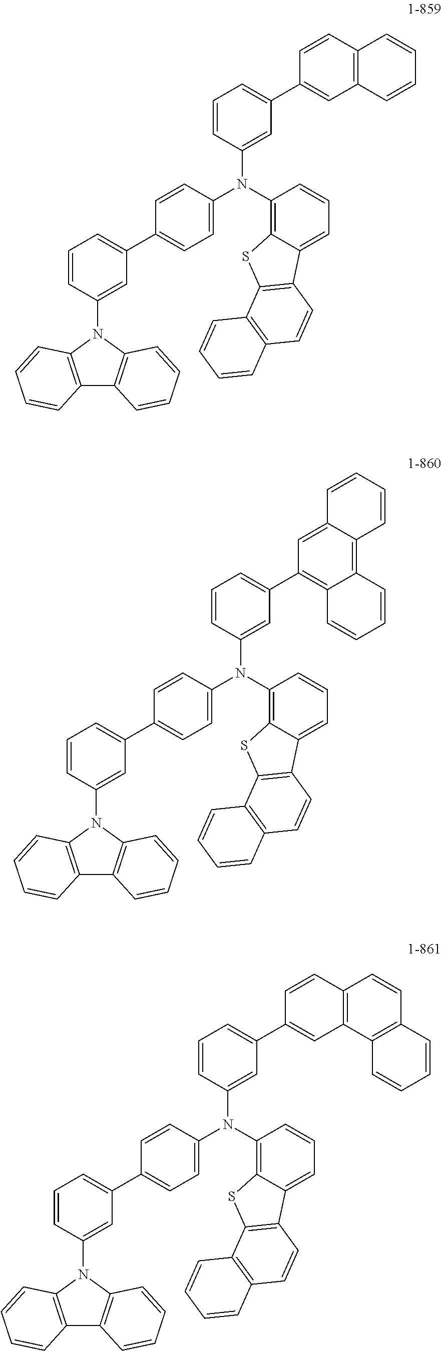 [SMILES: c1cc(-c2ccc3ccccc3c2)cc(N(c2ccc(-c3cccc(-n4c5ccccc5c5ccccc54)c3)cc2)c2cccc3c2sc2c4ccccc4ccc32)c1 ; c1cc(-c2cc3ccccc3c3ccccc23)cc(N(c2ccc(-c3cccc(-n4c5ccccc5c5ccccc54)c3)cc2)c2cccc3c2sc2c4ccccc4ccc32)c1 ; c1cc(-c2ccc3ccc4ccccc4c3c2)cc(N(c2ccc(-c3cccc(-n4c5ccccc5c5ccccc54)c3)cc2)c2cccc3c2sc2c4ccccc4ccc32)c1]

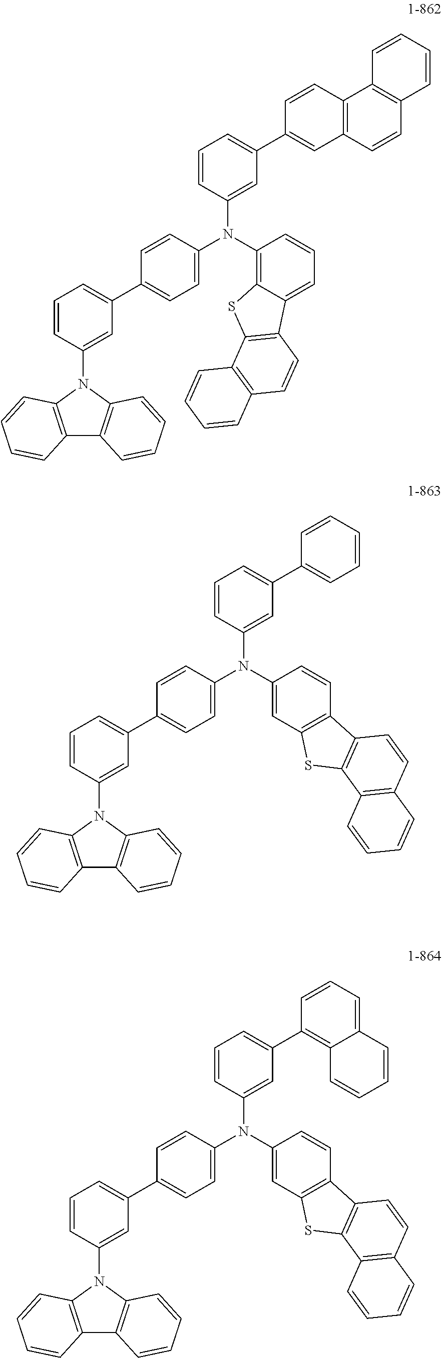 [SMILES: c1cc(-c2ccc3c(ccc4ccccc43)c2)cc(N(c2ccc(-c3cccc(-n4c5ccccc5c5ccccc54)c3)cc2)c2cccc3c2sc2c4ccccc4ccc32)c1 ; c1ccc(-c2cccc(N(c3ccc(-c4cccc(-n5c6ccccc6c6ccccc65)c4)cc3)c3ccc4c(c3)sc3c5ccccc5ccc43)c2)cc1 ; c1cc(-c2cccc3ccccc23)cc(N(c2ccc(-c3cccc(-n4c5ccccc5c5ccccc54)c3)cc2)c2ccc3c(c2)sc2c4ccccc4ccc32)c1]

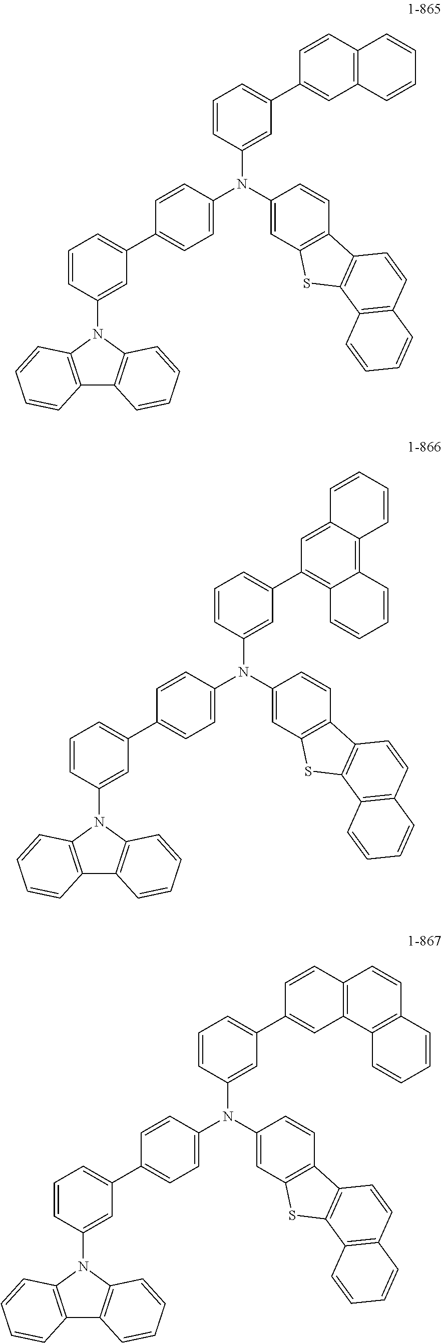 [SMILES: c1cc(-c2ccc3ccccc3c2)cc(N(c2ccc(-c3cccc(-n4c5ccccc5c5ccccc54)c3)cc2)c2ccc3c(c2)sc2c4ccccc4ccc32)c1 ; c1cc(-c2cc3ccccc3c3ccccc23)cc(N(c2ccc(-c3cccc(-n4c5ccccc5c5ccccc54)c3)cc2)c2ccc3c(c2)sc2c4ccccc4ccc32)c1 ; c1cc(-c2ccc3ccc4ccccc4c3c2)cc(N(c2ccc(-c3cccc(-n4c5ccccc5c5ccccc54)c3)cc2)c2ccc3c(c2)sc2c4ccccc4ccc32)c1]

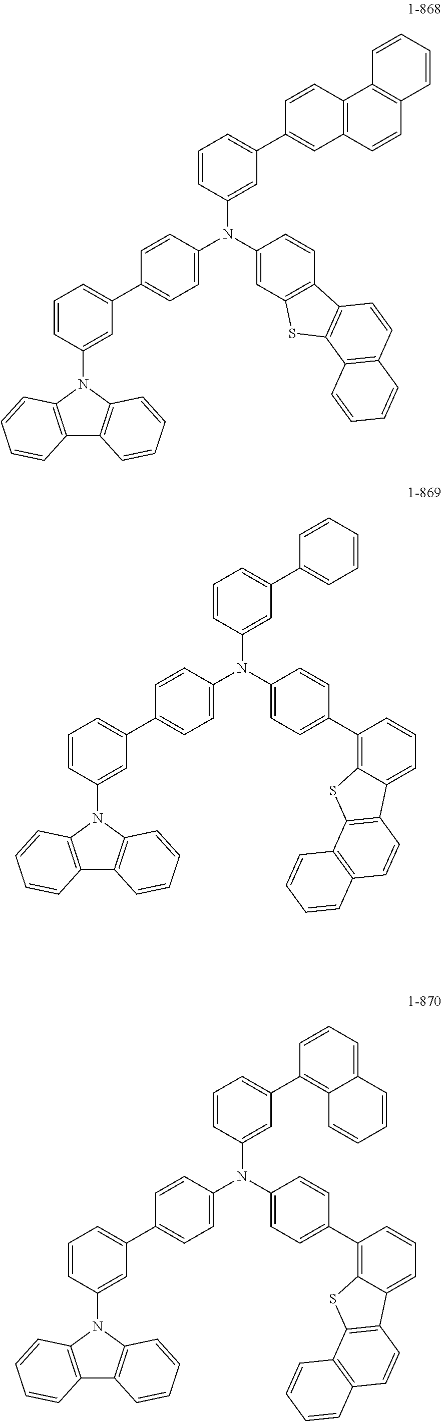 [SMILES: c1cc(-c2ccc3c(ccc4ccccc43)c2)cc(N(c2ccc(-c3cccc(-n4c5ccccc5c5ccccc54)c3)cc2)c2ccc3c(c2)sc2c4ccccc4ccc32)c1 ; c1ccc(-c2cccc(N(c3ccc(-c4cccc(-n5c6ccccc6c6ccccc65)c4)cc3)c3ccc(-c4cccc5c4sc4c6ccccc6ccc54)cc3)c2)cc1 ; c1cc(-c2cccc3ccccc23)cc(N(c2ccc(-c3cccc(-n4c5ccccc5c5ccccc54)c3)cc2)c2ccc(-c3cccc4c3sc3c5ccccc5ccc43)cc2)c1]

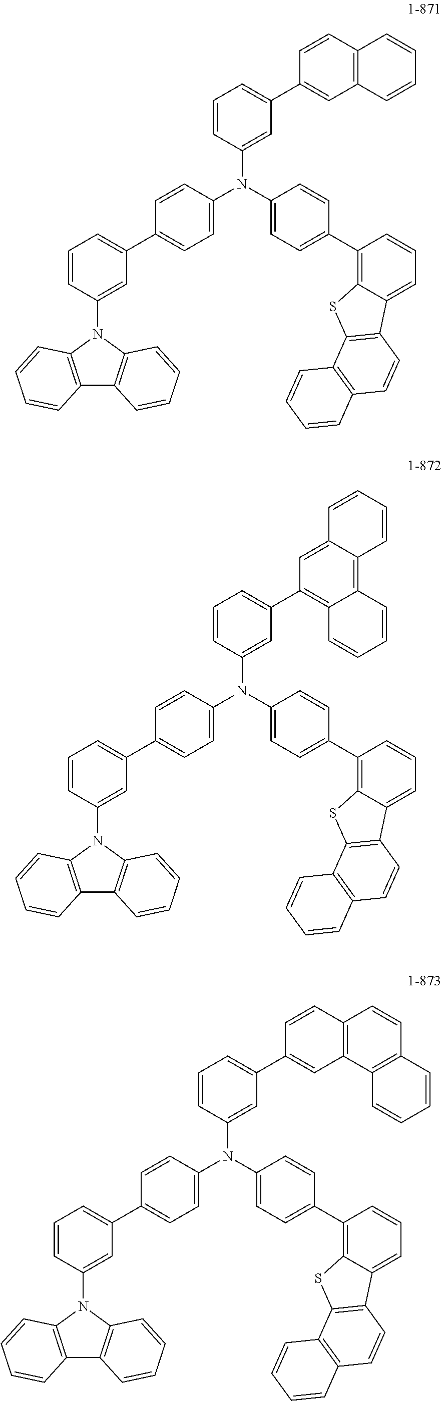 [SMILES: c1cc(-c2ccc3ccccc3c2)cc(N(c2ccc(-c3cccc(-n4c5ccccc5c5ccccc54)c3)cc2)c2ccc(-c3cccc4c3sc3c5ccccc5ccc43)cc2)c1 ; c1cc(-c2cc3ccccc3c3ccccc23)cc(N(c2ccc(-c3cccc(-n4c5ccccc5c5ccccc54)c3)cc2)c2ccc(-c3cccc4c3sc3c5ccccc5ccc43)cc2)c1 ; c1cc(-c2ccc3ccc4ccccc4c3c2)cc(N(c2ccc(-c3cccc(-n4c5ccccc5c5ccccc54)c3)cc2)c2ccc(-c3cccc4c3sc3c5ccccc5ccc43)cc2)c1]

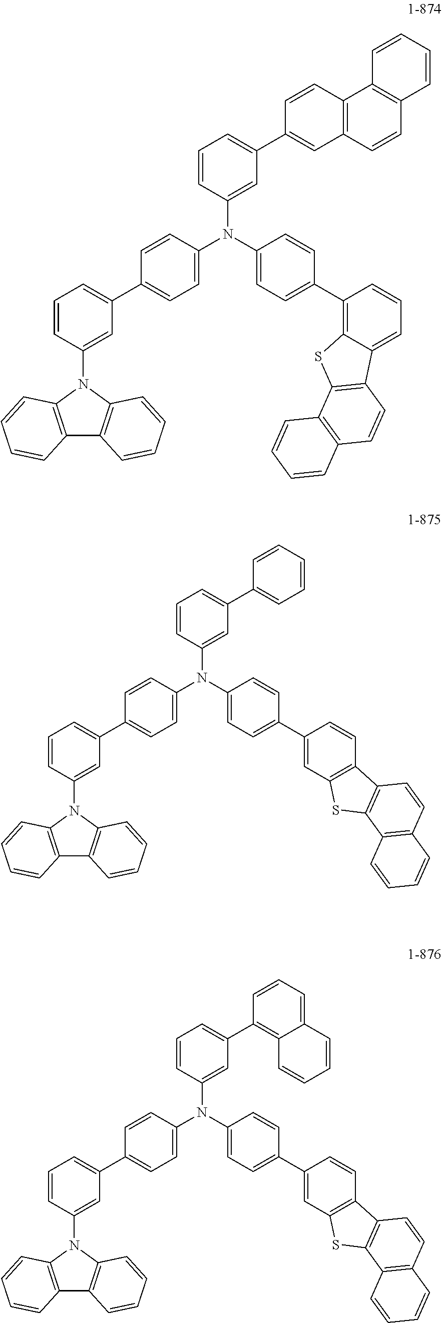 [SMILES: c1cc(-c2ccc3c(ccc4ccccc43)c2)cc(N(c2ccc(-c3cccc(-n4c5ccccc5c5ccccc54)c3)cc2)c2ccc(-c3cccc4c3sc3c5ccccc5ccc43)cc2)c1 ; c1ccc(-c2cccc(N(c3ccc(-c4cccc(-n5c6ccccc6c6ccccc65)c4)cc3)c3ccc(-c4ccc5c(c4)sc4c6ccccc6ccc54)cc3)c2)cc1 ; c1cc(-c2cccc3ccccc23)cc(N(c2ccc(-c3cccc(-n4c5ccccc5c5ccccc54)c3)cc2)c2ccc(-c3ccc4c(c3)sc3c5ccccc5ccc43)cc2)c1]

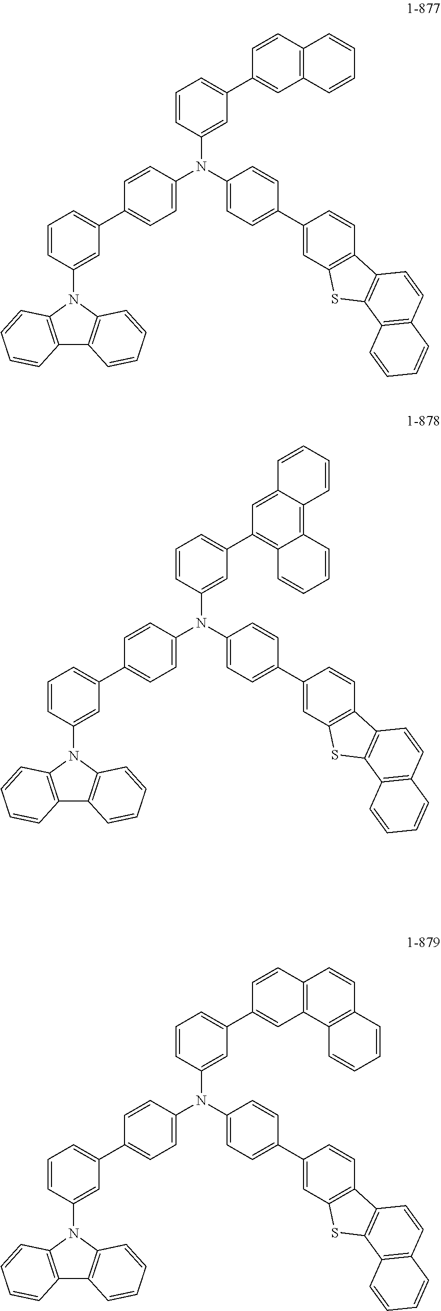 [SMILES: c1cc(-c2ccc3ccccc3c2)cc(N(c2ccc(-c3cccc(-n4c5ccccc5c5ccccc54)c3)cc2)c2ccc(-c3ccc4c(c3)sc3c5ccccc5ccc43)cc2)c1 ; c1cc(-c2cc3ccccc3c3ccccc23)cc(N(c2ccc(-c3cccc(-n4c5ccccc5c5ccccc54)c3)cc2)c2ccc(-c3ccc4c(c3)sc3c5ccccc5ccc43)cc2)c1 ; c1cc(-c2ccc3ccc4ccccc4c3c2)cc(N(c2ccc(-c3cccc(-n4c5ccccc5c5ccccc54)c3)cc2)c2ccc(-c3ccc4c(c3)sc3c5ccccc5ccc43)cc2)c1]

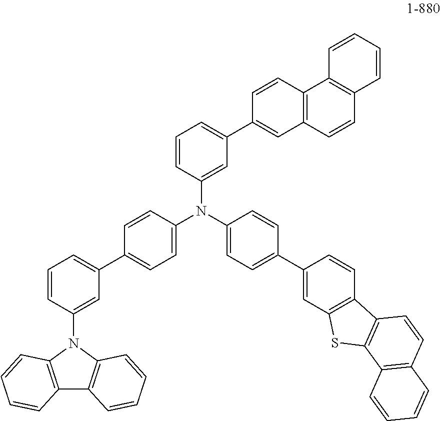 [SMILES: c1cc(-c2ccc3c(ccc4ccccc43)c2)cc(N(c2ccc(-c3cccc(-n4c5ccccc5c5ccccc54)c3)cc2)c2ccc(-c3ccc4c(c3)sc3c5ccccc5ccc43)cc2)c1]

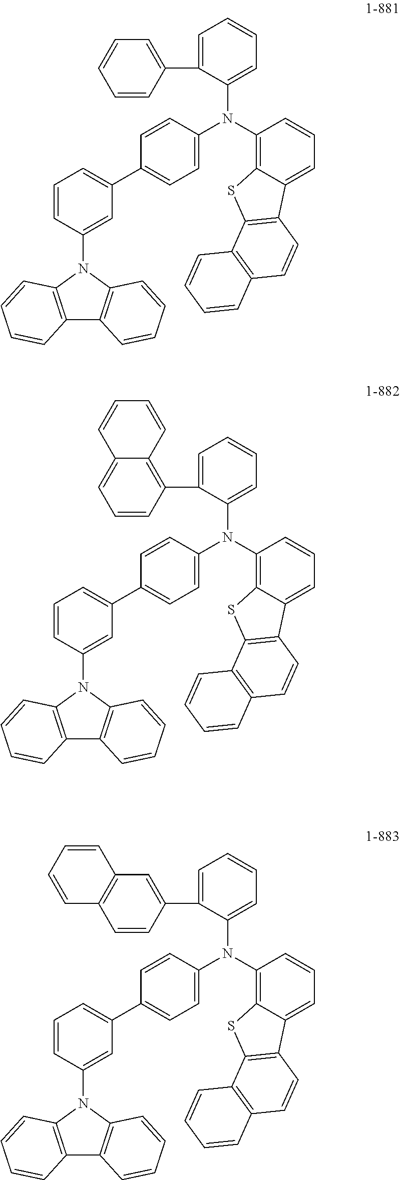 [SMILES: c1ccc(-c2ccccc2N(c2ccc(-c3cccc(-n4c5ccccc5c5ccccc54)c3)cc2)c2cccc3c2sc2c4ccccc4ccc32)cc1 ; c1cc(-c2ccc(N(c3ccccc3-c3cccc4ccccc34)c3cccc4c3sc3c5ccccc5ccc43)cc2)cc(-n2c3ccccc3c3ccccc32)c1 ; c1cc(-c2ccc(N(c3ccccc3-c3ccc4ccccc4c3)c3cccc4c3sc3c5ccccc5ccc43)cc2)cc(-n2c3ccccc3c3ccccc32)c1]

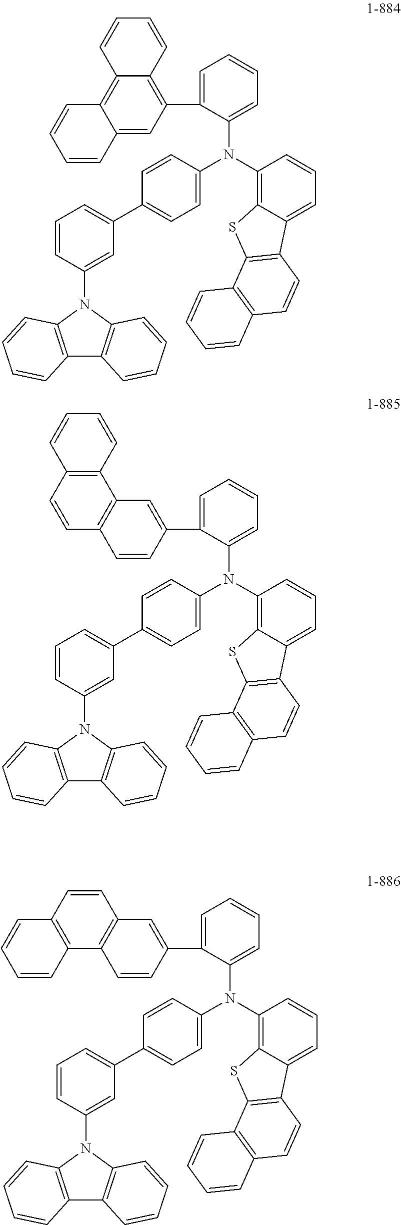

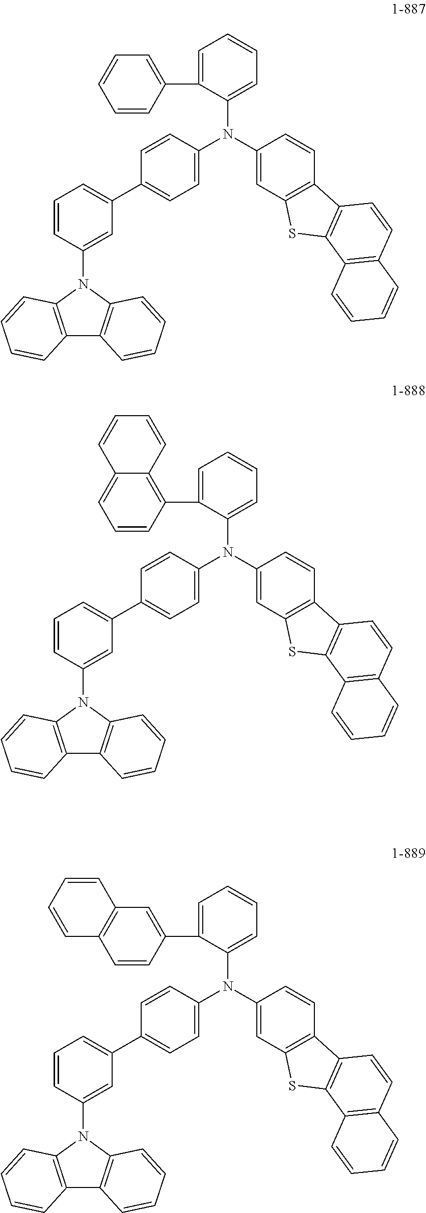 [SMILES: c1ccc(-c2ccccc2N(c2ccc(-c3cccc(-n4c5ccccc5c5ccccc54)c3)cc2)c2ccc3c(c2)sc2c4ccccc4ccc32)cc1 ; c1cc(-c2ccc(N(c3ccc4c(c3)sc3c5ccccc5ccc43)c3ccccc3-c3cccc4ccccc34)cc2)cc(-n2c3ccccc3c3ccccc32)c1 ; c1cc(-c2ccc(N(c3ccc4c(c3)sc3c5ccccc5ccc43)c3ccccc3-c3ccc4ccccc4c3)cc2)cc(-n2c3ccccc3c3ccccc32)c1]

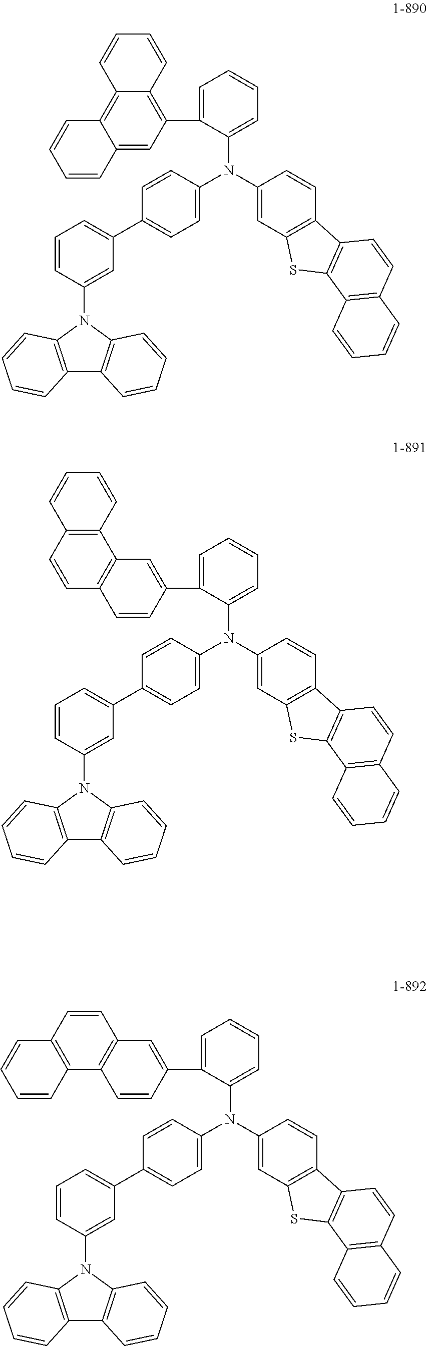 [SMILES: c1cc(-c2ccc(N(c3ccc4c(c3)sc3c5ccccc5ccc43)c3ccccc3-c3cc4ccccc4c4ccccc34)cc2)cc(-n2c3ccccc3c3ccccc32)c1 ; c1cc(-c2ccc(N(c3ccc4c(c3)sc3c5ccccc5ccc43)c3ccccc3-c3ccc4ccc5ccccc5c4c3)cc2)cc(-n2c3ccccc3c3ccccc32)c1 ; c1cc(-c2ccc(N(c3ccc4c(c3)sc3c5ccccc5ccc43)c3ccccc3-c3ccc4c(ccc5ccccc54)c3)cc2)cc(-n2c3ccccc3c3ccccc32)c1]

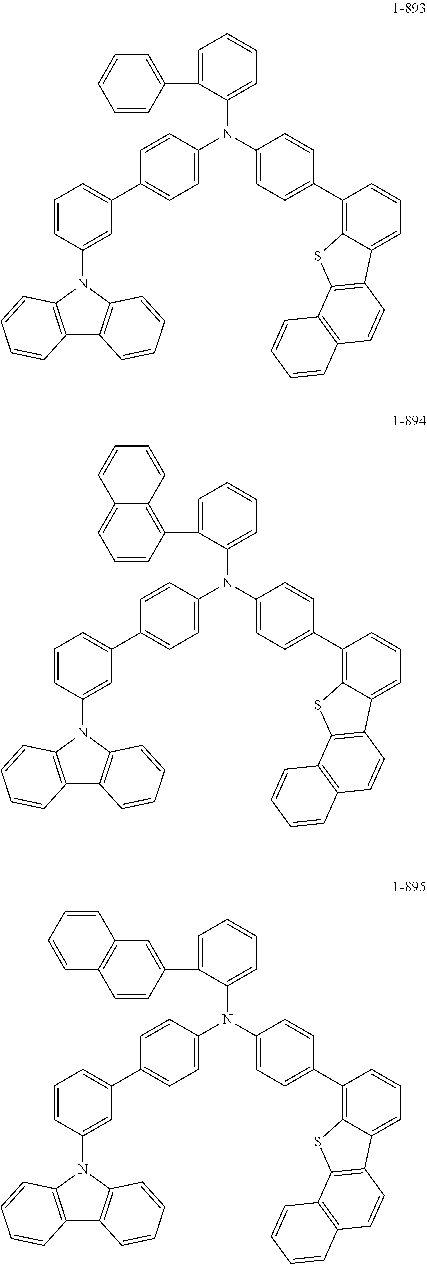 [SMILES: c1ccc(-c2ccccc2N(c2ccc(-c3cccc(-n4c5ccccc5c5ccccc54)c3)cc2)c2ccc(-c3cccc4c3sc3c5ccccc5ccc43)cc2)cc1 ; c1cc(-c2ccc(N(c3ccc(-c4cccc5c4sc4c6ccccc6ccc54)cc3)c3ccccc3-c3cccc4ccccc34)cc2)cc(-n2c3ccccc3c3ccccc32)c1 ; c1cc(-c2ccc(N(c3ccc(-c4cccc5c4sc4c6ccccc6ccc54)cc3)c3ccccc3-c3ccc4ccccc4c3)cc2)cc(-n2c3ccccc3c3ccccc32)c1]

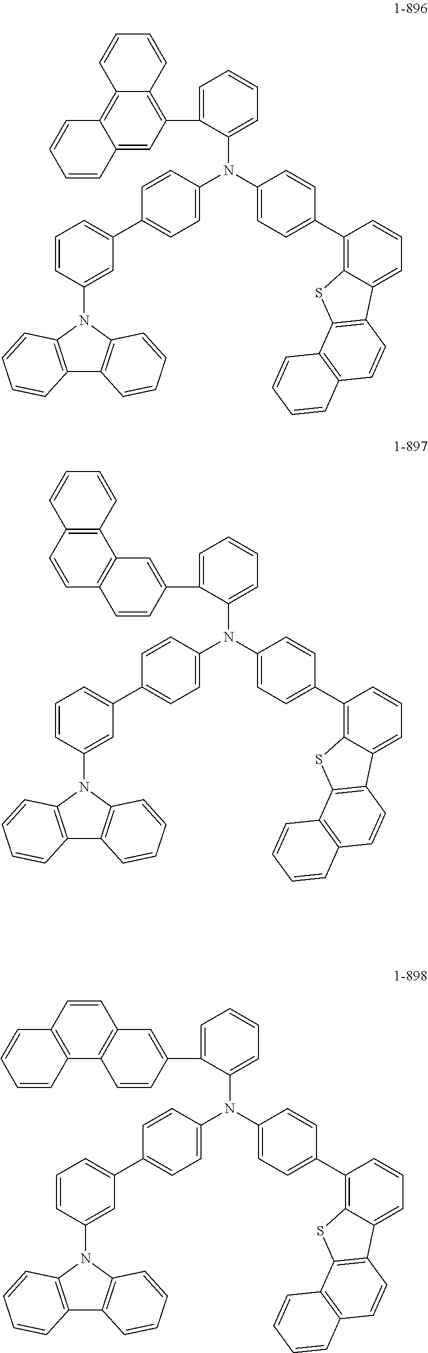 [SMILES: c1cc(-c2ccc(N(c3ccc(-c4cccc5c4sc4c6ccccc6ccc54)cc3)c3ccccc3-c3cc4ccccc4c4ccccc34)cc2)cc(-n2c3ccccc3c3ccccc32)c1 ; c1cc(-c2ccc(N(c3ccc(-c4cccc5c4sc4c6ccccc6ccc54)cc3)c3ccccc3-c3ccc4ccc5ccccc5c4c3)cc2)cc(-n2c3ccccc3c3ccccc32)c1 ; c1cc(-c2ccc(N(c3ccc(-c4cccc5c4sc4c6ccccc6ccc54)cc3)c3ccccc3-c3ccc4c(ccc5ccccc54)c3)cc2)cc(-n2c3ccccc3c3ccccc32)c1]

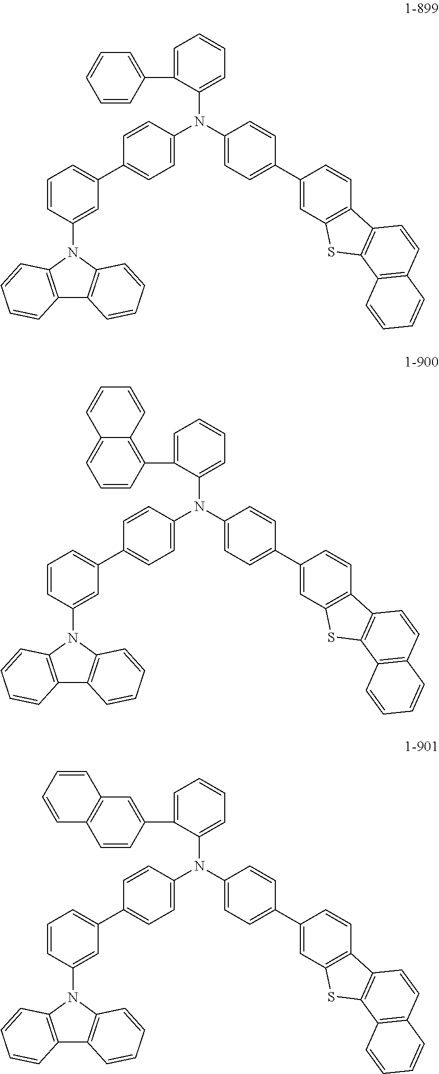 [SMILES: c1ccc(-c2ccccc2N(c2ccc(-c3cccc(-n4c5ccccc5c5ccccc54)c3)cc2)c2ccc(-c3ccc4c(c3)sc3c5ccccc5ccc43)cc2)cc1 ; c1cc(-c2ccc(N(c3ccc(-c4ccc5c(c4)sc4c6ccccc6ccc54)cc3)c3ccccc3-c3cccc4ccccc34)cc2)cc(-n2c3ccccc3c3ccccc32)c1 ; c1cc(-c2ccc(N(c3ccc(-c4ccc5c(c4)sc4c6ccccc6ccc54)cc3)c3ccccc3-c3ccc4ccccc4c3)cc2)cc(-n2c3ccccc3c3ccccc32)c1]

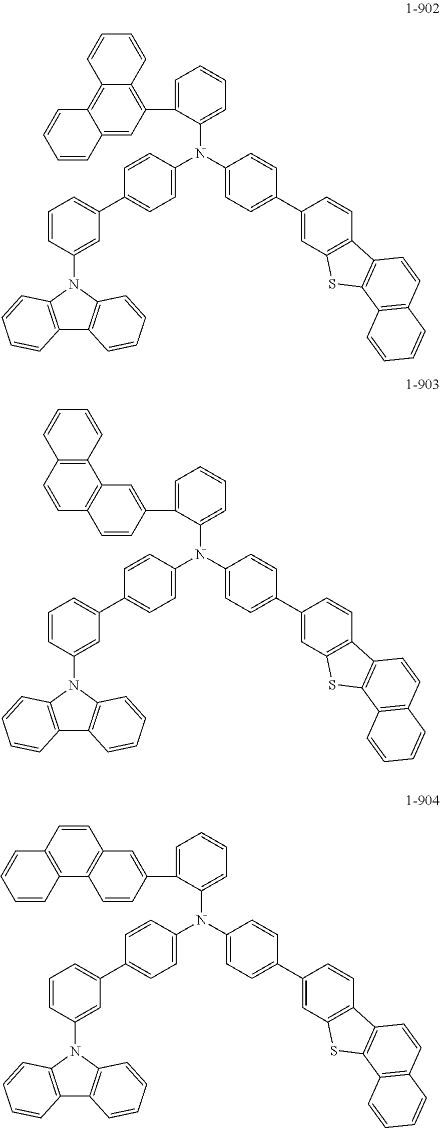 [SMILES: c1cc(-c2ccc(N(c3ccc(-c4ccc5c(c4)sc4c6ccccc6ccc54)cc3)c3ccccc3-c3cc4ccccc4c4ccccc34)cc2)cc(-n2c3ccccc3c3ccccc32)c1 ; c1cc(-c2ccc(N(c3ccc(-c4ccc5c(c4)sc4c6ccccc6ccc54)cc3)c3ccccc3-c3ccc4ccc5ccccc5c4c3)cc2)cc(-n2c3ccccc3c3ccccc32)c1 ; c1cc(-c2ccc(N(c3ccc(-c4ccc5c(c4)sc4c6ccccc6ccc54)cc3)c3ccccc3-c3ccc4c(ccc5ccccc54)c3)cc2)cc(-n2c3ccccc3c3ccccc32)c1]

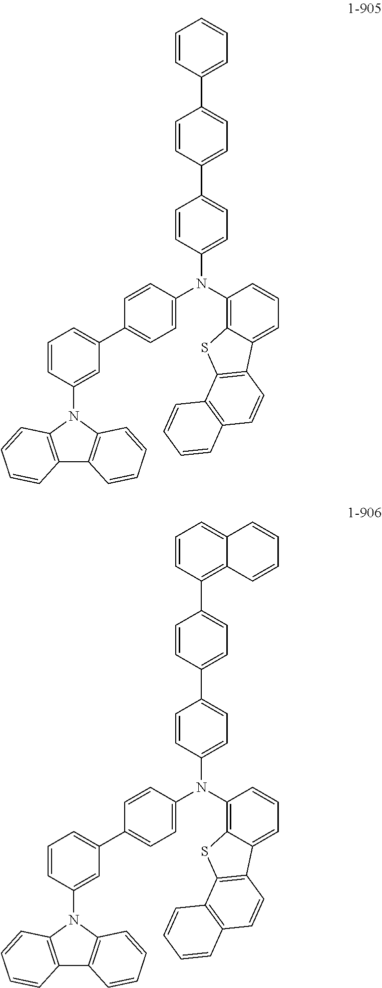 [SMILES: c1ccc(-c2ccc(-c3ccc(N(c4ccc(-c5cccc(-n6c7ccccc7c7ccccc76)c5)cc4)c4cccc5c4sc4c6ccccc6ccc54)cc3)cc2)cc1 ; c1cc(-c2ccc(N(c3ccc(-c4ccc(-c5cccc6ccccc56)cc4)cc3)c3cccc4c3sc3c5ccccc5ccc43)cc2)cc(-n2c3ccccc3c3ccccc32)c1]

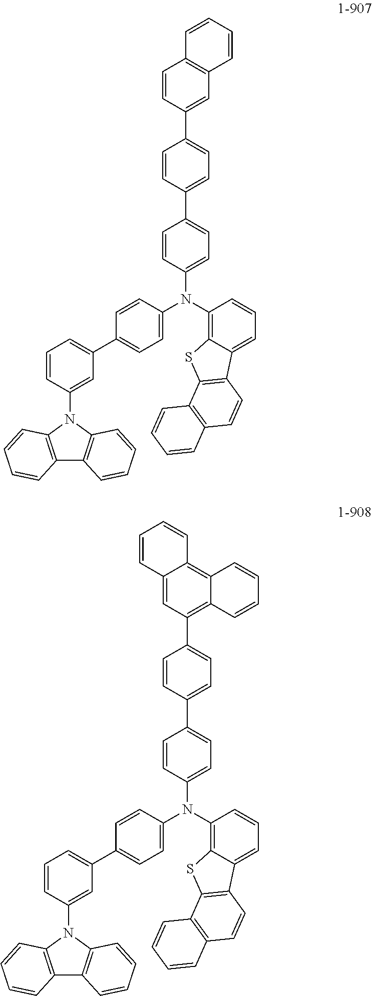 [SMILES: c1cc(-c2ccc(N(c3ccc(-c4ccc(-c5ccc6ccccc6c5)cc4)cc3)c3cccc4c3sc3c5ccccc5ccc43)cc2)cc(-n2c3ccccc3c3ccccc32)c1 ; c1cc(-c2ccc(N(c3ccc(-c4ccc(-c5cc6ccccc6c6ccccc56)cc4)cc3)c3cccc4c3sc3c5ccccc5ccc43)cc2)cc(-n2c3ccccc3c3ccccc32)c1]

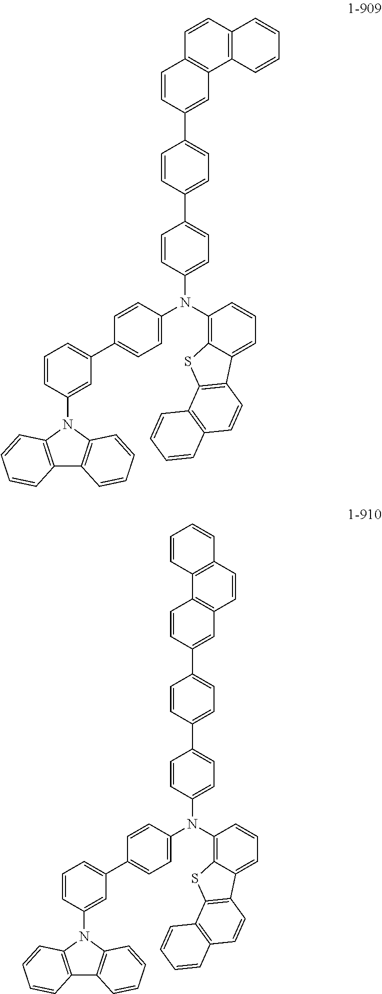 [SMILES: c1cc(-c2ccc(N(c3ccc(-c4ccc(-c5ccc6ccc7ccccc7c6c5)cc4)cc3)c3cccc4c3sc3c5ccccc5ccc43)cc2)cc(-n2c3ccccc3c3ccccc32)c1 ; c1cc(-c2ccc(N(c3ccc(-c4ccc(-c5ccc6c(ccc7ccccc76)c5)cc4)cc3)c3cccc4c3sc3c5ccccc5ccc43)cc2)cc(-n2c3ccccc3c3ccccc32)c1]

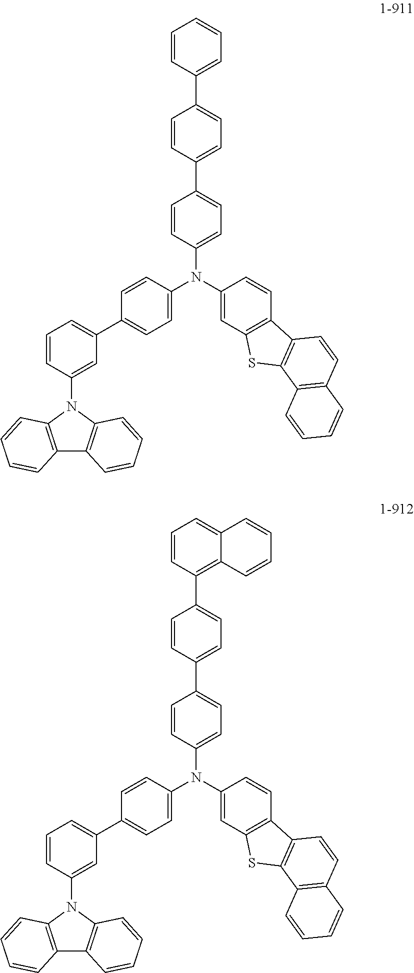 [SMILES: c1ccc(-c2ccc(-c3ccc(N(c4ccc(-c5cccc(-n6c7ccccc7c7ccccc76)c5)cc4)c4ccc5c(c4)sc4c6ccccc6ccc54)cc3)cc2)cc1 ; c1cc(-c2ccc(N(c3ccc(-c4ccc(-c5cccc6ccccc56)cc4)cc3)c3ccc4c(c3)sc3c5ccccc5ccc43)cc2)cc(-n2c3ccccc3c3ccccc32)c1]

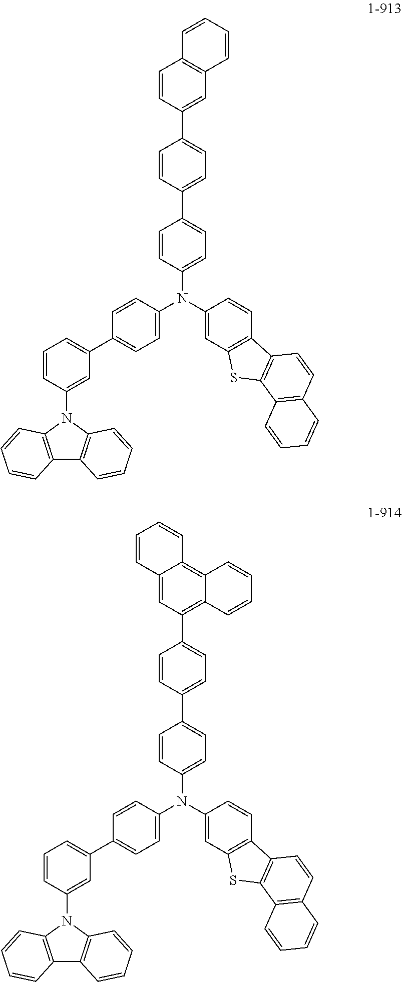 [SMILES: c1cc(-c2ccc(N(c3ccc(-c4ccc(-c5ccc6ccccc6c5)cc4)cc3)c3ccc4c(c3)sc3c5ccccc5ccc43)cc2)cc(-n2c3ccccc3c3ccccc32)c1 ; c1cc(-c2ccc(N(c3ccc(-c4ccc(-c5cc6ccccc6c6ccccc56)cc4)cc3)c3ccc4c(c3)sc3c5ccccc5ccc43)cc2)cc(-n2c3ccccc3c3ccccc32)c1]

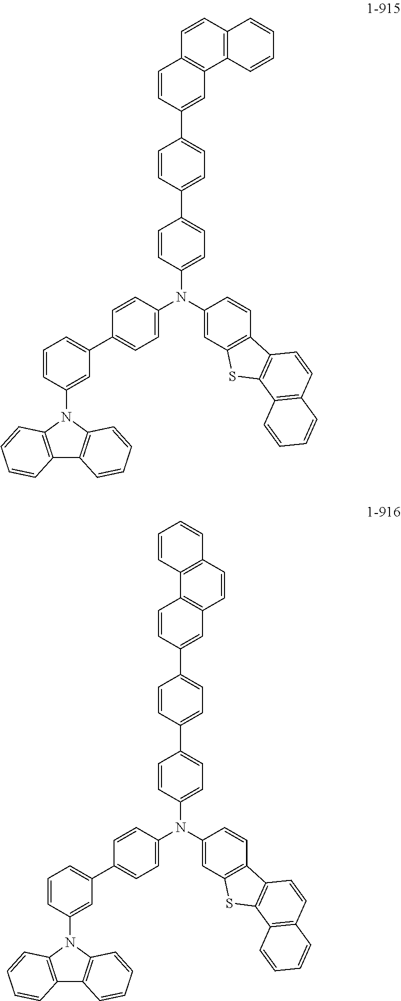 [SMILES: c1cc(-c2ccc(N(c3ccc(-c4ccc(-c5ccc6ccc7ccccc7c6c5)cc4)cc3)c3ccc4c(c3)sc3c5ccccc5ccc43)cc2)cc(-n2c3ccccc3c3ccccc32)c1 ; c1cc(-c2ccc(N(c3ccc(-c4ccc(-c5ccc6c(ccc7ccccc76)c5)cc4)cc3)c3ccc4c(c3)sc3c5ccccc5ccc43)cc2)cc(-n2c3ccccc3c3ccccc32)c1]

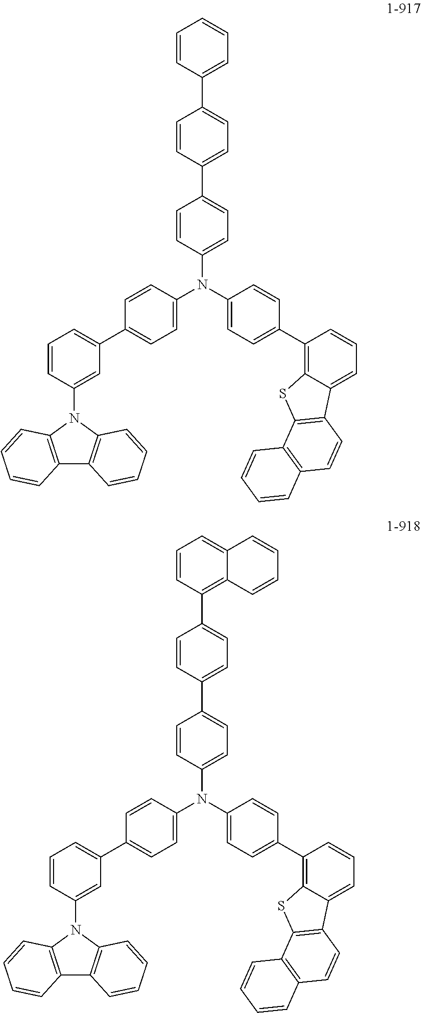

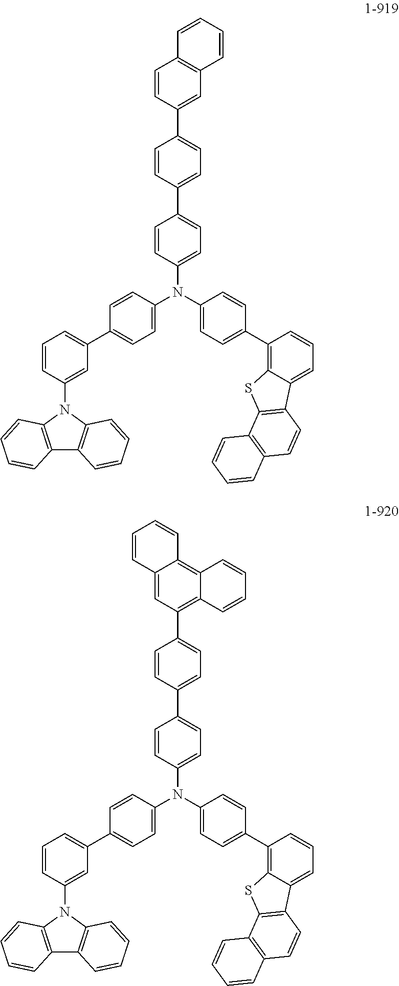 [SMILES: c1cc(-c2ccc(N(c3ccc(-c4ccc(-c5ccc6ccccc6c5)cc4)cc3)c3ccc(-c4cccc5c4sc4c6ccccc6ccc54)cc3)cc2)cc(-n2c3ccccc3c3ccccc32)c1 ; c1cc(-c2ccc(N(c3ccc(-c4ccc(-c5cc6ccccc6c6ccccc56)cc4)cc3)c3ccc(-c4cccc5c4sc4c6ccccc6ccc54)cc3)cc2)cc(-n2c3ccccc3c3ccccc32)c1]

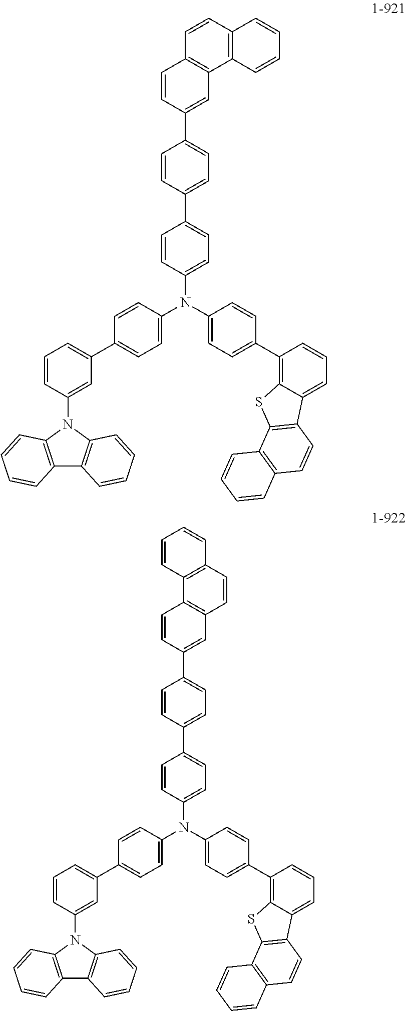 [SMILES: c1cc(-c2ccc(N(c3ccc(-c4ccc(-c5ccc6ccc7ccccc7c6c5)cc4)cc3)c3ccc(-c4cccc5c4sc4c6ccccc6ccc54)cc3)cc2)cc(-n2c3ccccc3c3ccccc32)c1 ; c1cc(-c2ccc(N(c3ccc(-c4ccc(-c5ccc6c(ccc7ccccc76)c5)cc4)cc3)c3ccc(-c4cccc5c4sc4c6ccccc6ccc54)cc3)cc2)cc(-n2c3ccccc3c3ccccc32)c1]

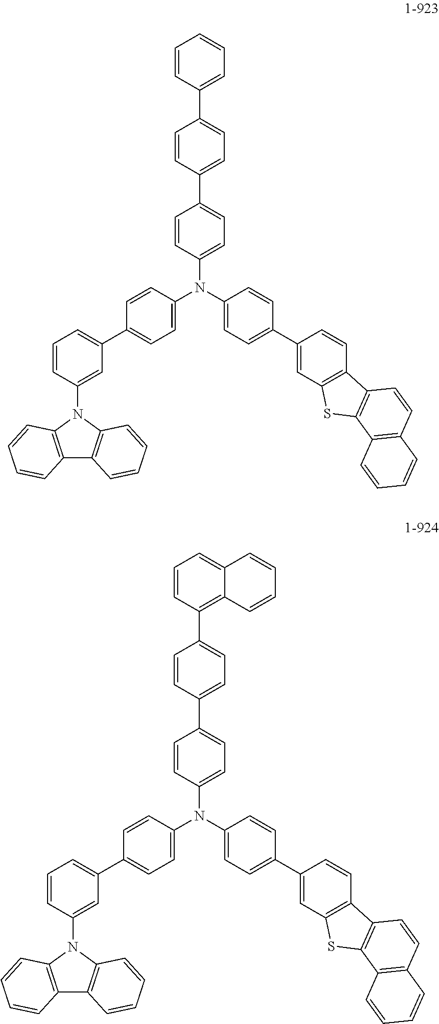 [SMILES: c1ccc(-c2ccc(-c3ccc(N(c4ccc(-c5cccc(-n6c7ccccc7c7ccccc76)c5)cc4)c4ccc(-c5ccc6c(c5)sc5c7ccccc7ccc65)cc4)cc3)cc2)cc1 ; c1cc(-c2ccc(N(c3ccc(-c4ccc(-c5cccc6ccccc56)cc4)cc3)c3ccc(-c4ccc5c(c4)sc4c6ccccc6ccc54)cc3)cc2)cc(-n2c3ccccc3c3ccccc32)c1]

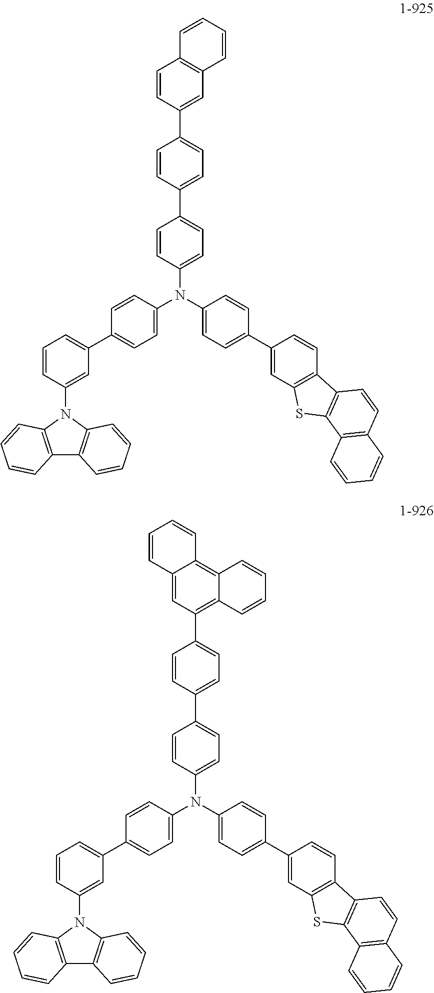 [SMILES: c1cc(-c2ccc(N(c3ccc(-c4ccc(-c5ccc6ccccc6c5)cc4)cc3)c3ccc(-c4ccc5c(c4)sc4c6ccccc6ccc54)cc3)cc2)cc(-n2c3ccccc3c3ccccc32)c1 ; c1cc(-c2ccc(N(c3ccc(-c4ccc(-c5cc6ccccc6c6ccccc56)cc4)cc3)c3ccc(-c4ccc5c(c4)sc4c6ccccc6ccc54)cc3)cc2)cc(-n2c3ccccc3c3ccccc32)c1]

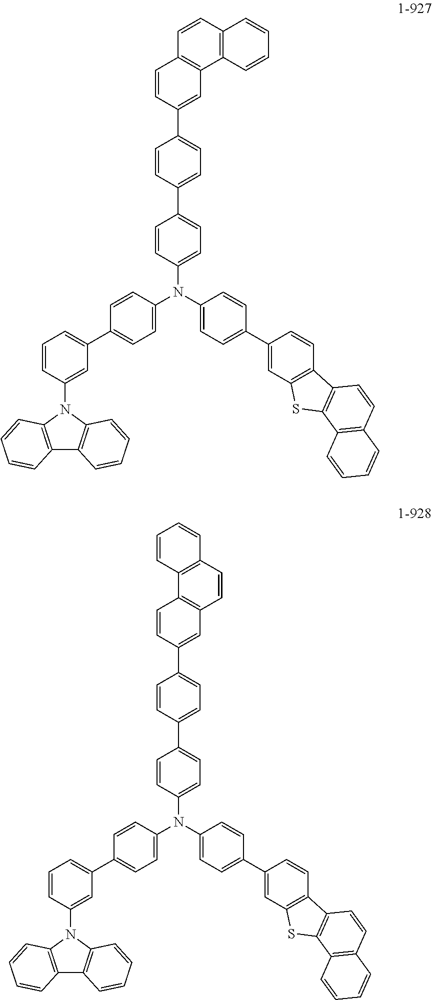 [SMILES: c1cc(-c2ccc(N(c3ccc(-c4ccc(-c5ccc6ccc7ccccc7c6c5)cc4)cc3)c3ccc(-c4ccc5c(c4)sc4c6ccccc6ccc54)cc3)cc2)cc(-n2c3ccccc3c3ccccc32)c1 ; c1cc(-c2ccc(N(c3ccc(-c4ccc(-c5ccc6c(ccc7ccccc76)c5)cc4)cc3)c3ccc(-c4ccc5c(c4)sc4c6ccccc6ccc54)cc3)cc2)cc(-n2c3ccccc3c3ccccc32)c1]

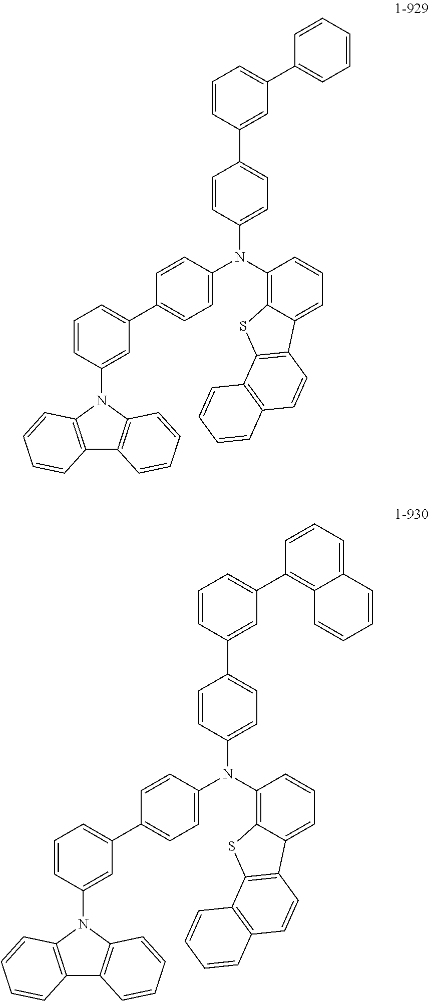 [SMILES: c1ccc(-c2cccc(-c3ccc(N(c4ccc(-c5cccc(-n6c7ccccc7c7ccccc76)c5)cc4)c4cccc5c4sc4c6ccccc6ccc54)cc3)c2)cc1 ; c1cc(-c2ccc(N(c3ccc(-c4cccc(-n5c6ccccc6c6ccccc65)c4)cc3)c3cccc4c3sc3c5ccccc5ccc43)cc2)cc(-c2cccc3ccccc23)c1]

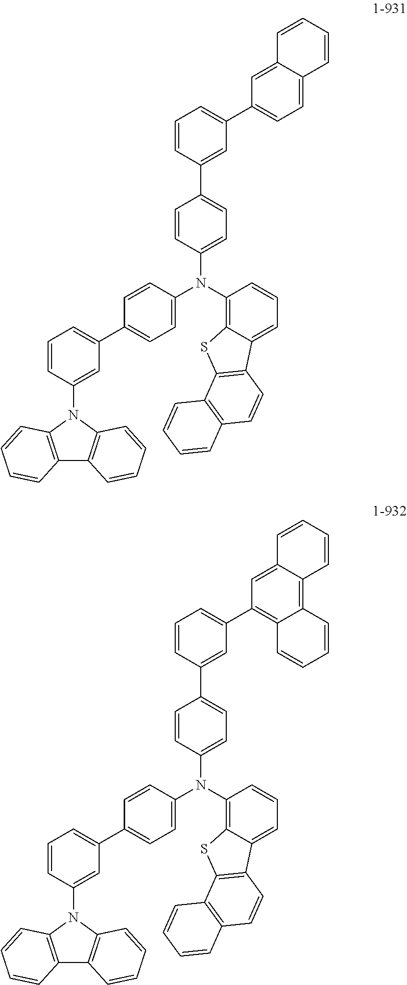 [SMILES: c1cc(-c2ccc(N(c3ccc(-c4cccc(-n5c6ccccc6c6ccccc65)c4)cc3)c3cccc4c3sc3c5ccccc5ccc43)cc2)cc(-c2ccc3ccccc3c2)c1 ; c1cc(-c2ccc(N(c3ccc(-c4cccc(-n5c6ccccc6c6ccccc65)c4)cc3)c3cccc4c3sc3c5ccccc5ccc43)cc2)cc(-c2cc3ccccc3c3ccccc23)c1]

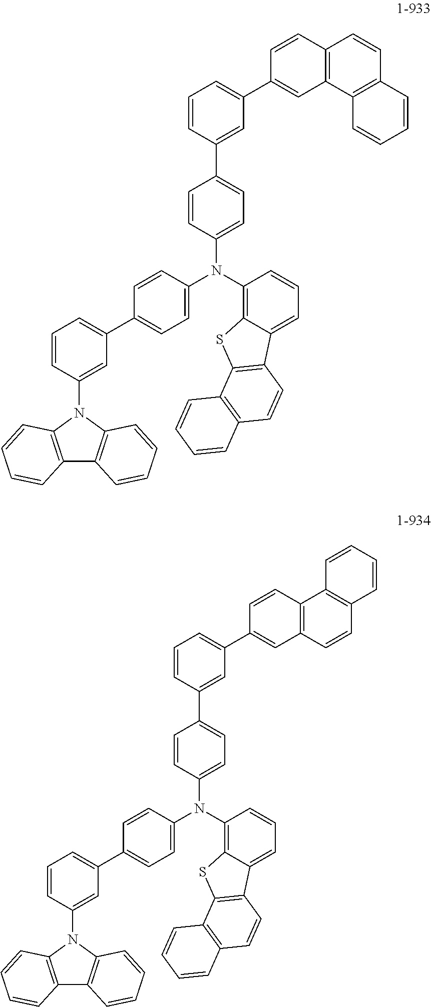 [SMILES: c1cc(-c2ccc(N(c3ccc(-c4cccc(-n5c6ccccc6c6ccccc65)c4)cc3)c3cccc4c3sc3c5ccccc5ccc43)cc2)cc(-c2ccc3ccc4ccccc4c3c2)c1 ; c1cc(-c2ccc(N(c3ccc(-c4cccc(-n5c6ccccc6c6ccccc65)c4)cc3)c3cccc4c3sc3c5ccccc5ccc43)cc2)cc(-c2ccc3c(ccc4ccccc43)c2)c1]

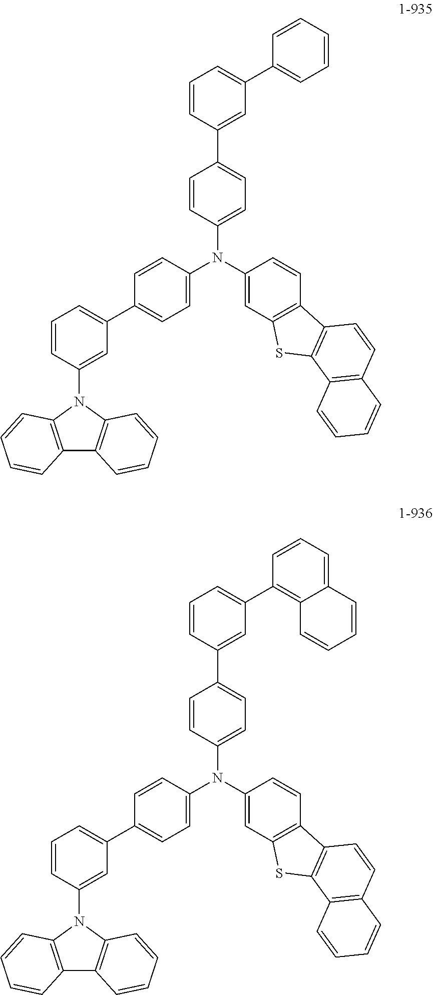 [SMILES: c1ccc(-c2cccc(-c3ccc(N(c4ccc(-c5cccc(-n6c7ccccc7c7ccccc76)c5)cc4)c4ccc5c(c4)sc4c6ccccc6ccc54)cc3)c2)cc1 ; c1cc(-c2ccc(N(c3ccc(-c4cccc(-n5c6ccccc6c6ccccc65)c4)cc3)c3ccc4c(c3)sc3c5ccccc5ccc43)cc2)cc(-c2cccc3ccccc23)c1]

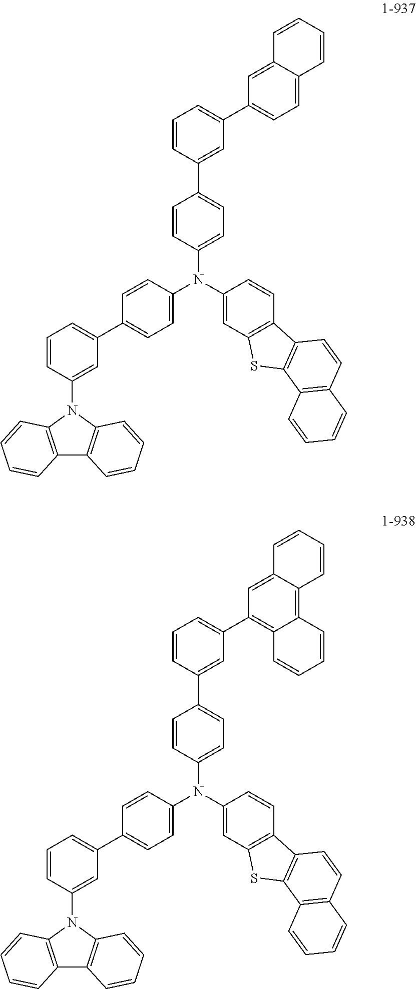 [SMILES: c1cc(-c2ccc(N(c3ccc(-c4cccc(-n5c6ccccc6c6ccccc65)c4)cc3)c3ccc4c(c3)sc3c5ccccc5ccc43)cc2)cc(-c2ccc3ccccc3c2)c1 ; c1cc(-c2ccc(N(c3ccc(-c4cccc(-n5c6ccccc6c6ccccc65)c4)cc3)c3ccc4c(c3)sc3c5ccccc5ccc43)cc2)cc(-c2cc3ccccc3c3ccccc23)c1]

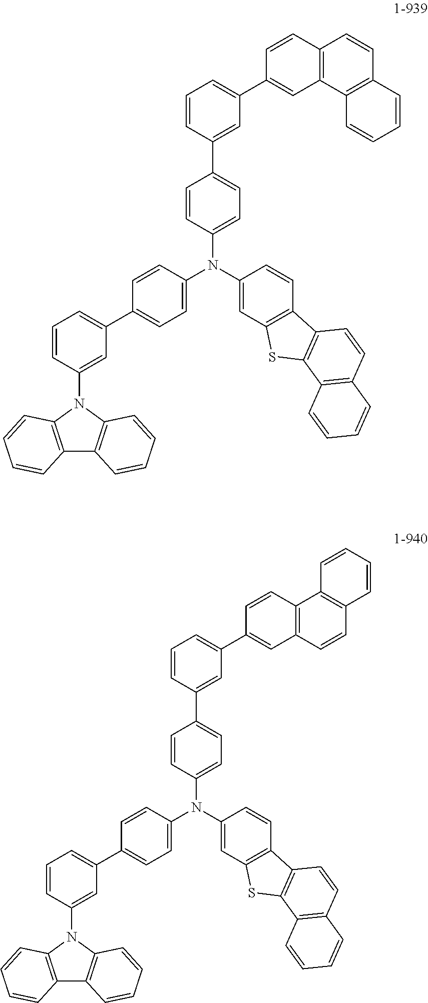 [SMILES: c1cc(-c2ccc(N(c3ccc(-c4cccc(-n5c6ccccc6c6ccccc65)c4)cc3)c3ccc4c(c3)sc3c5ccccc5ccc43)cc2)cc(-c2ccc3ccc4ccccc4c3c2)c1 ; c1cc(-c2ccc(N(c3ccc(-c4cccc(-n5c6ccccc6c6ccccc65)c4)cc3)c3ccc4c(c3)sc3c5ccccc5ccc43)cc2)cc(-c2ccc3c(ccc4ccccc43)c2)c1]

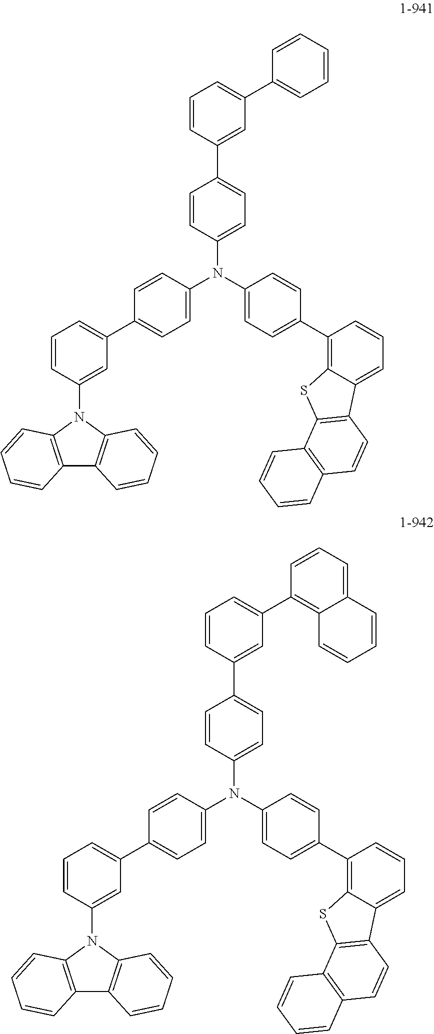

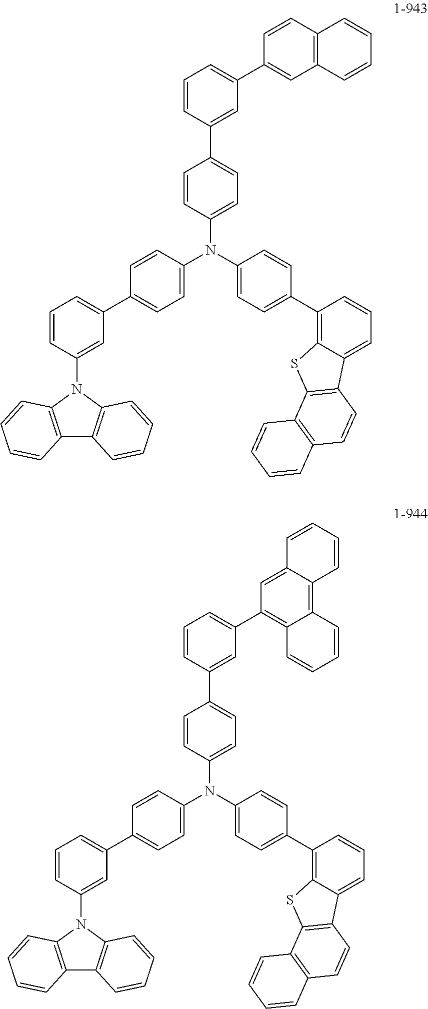 [SMILES: c1cc(-c2ccc(N(c3ccc(-c4cccc(-n5c6ccccc6c6ccccc65)c4)cc3)c3ccc(-c4cccc5c4sc4c6ccccc6ccc54)cc3)cc2)cc(-c2ccc3ccccc3c2)c1 ; c1cc(-c2ccc(N(c3ccc(-c4cccc(-n5c6ccccc6c6ccccc65)c4)cc3)c3ccc(-c4cccc5c4sc4c6ccccc6ccc54)cc3)cc2)cc(-c2cc3ccccc3c3ccccc23)c1]

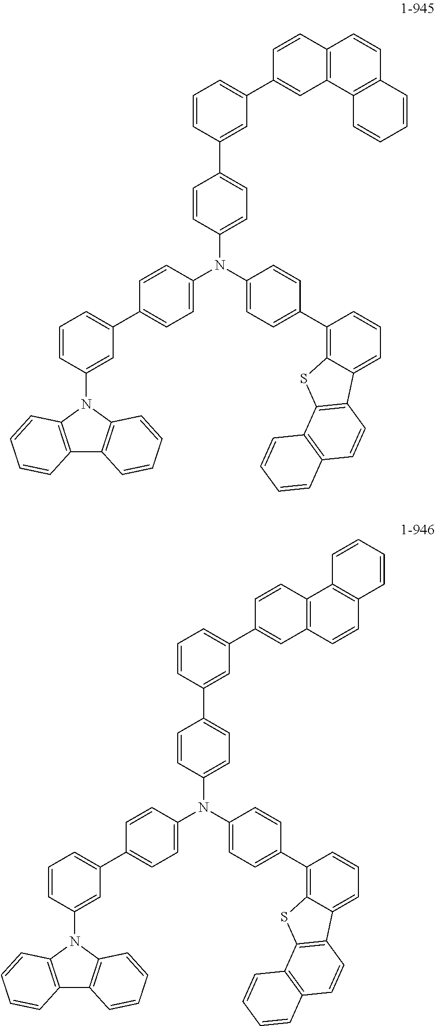 [SMILES: c1cc(-c2ccc(N(c3ccc(-c4cccc(-n5c6ccccc6c6ccccc65)c4)cc3)c3ccc(-c4cccc5c4sc4c6ccccc6ccc54)cc3)cc2)cc(-c2ccc3ccc4ccccc4c3c2)c1 ; c1cc(-c2ccc(N(c3ccc(-c4cccc(-n5c6ccccc6c6ccccc65)c4)cc3)c3ccc(-c4cccc5c4sc4c6ccccc6ccc54)cc3)cc2)cc(-c2ccc3c(ccc4ccccc43)c2)c1]

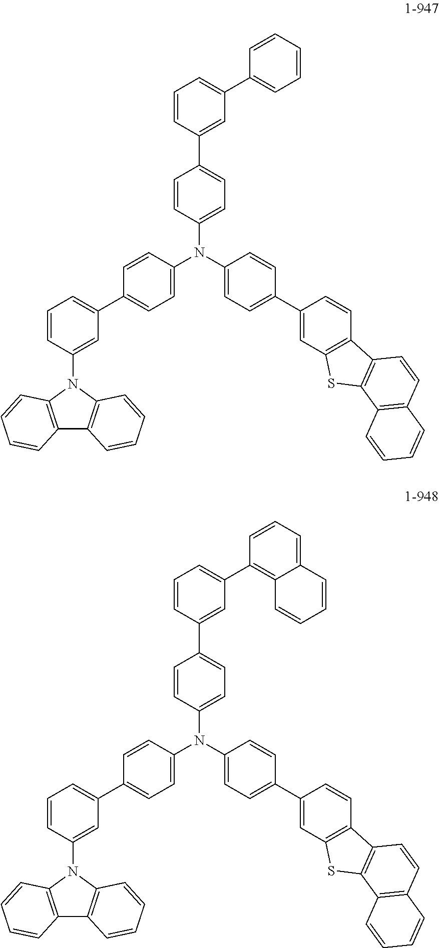 [SMILES: c1ccc(-c2cccc(-c3ccc(N(c4ccc(-c5cccc(-n6c7ccccc7c7ccccc76)c5)cc4)c4ccc(-c5ccc6c(c5)sc5c7ccccc7ccc65)cc4)cc3)c2)cc1 ; c1cc(-c2ccc(N(c3ccc(-c4cccc(-n5c6ccccc6c6ccccc65)c4)cc3)c3ccc(-c4ccc5c(c4)sc4c6ccccc6ccc54)cc3)cc2)cc(-c2cccc3ccccc23)c1]

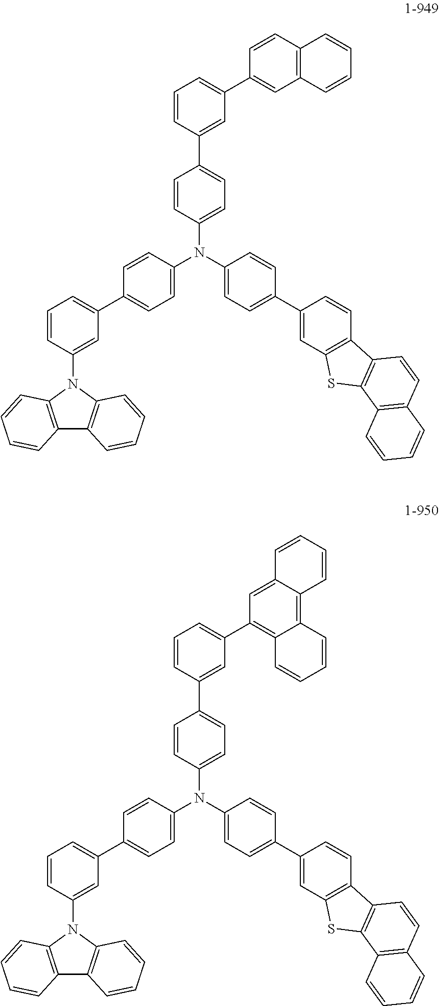 [SMILES: c1cc(-c2ccc(N(c3ccc(-c4cccc(-n5c6ccccc6c6ccccc65)c4)cc3)c3ccc(-c4ccc5c(c4)sc4c6ccccc6ccc54)cc3)cc2)cc(-c2ccc3ccccc3c2)c1 ; c1cc(-c2ccc(N(c3ccc(-c4cccc(-n5c6ccccc6c6ccccc65)c4)cc3)c3ccc(-c4ccc5c(c4)sc4c6ccccc6ccc54)cc3)cc2)cc(-c2cc3ccccc3c3ccccc23)c1]

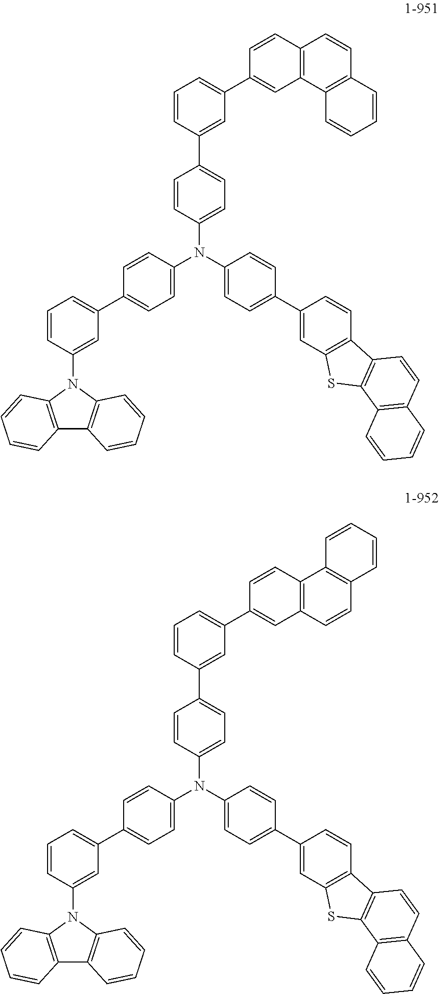 [SMILES: c1cc(-c2ccc(N(c3ccc(-c4cccc(-n5c6ccccc6c6ccccc65)c4)cc3)c3ccc(-c4ccc5c(c4)sc4c6ccccc6ccc54)cc3)cc2)cc(-c2ccc3ccc4ccccc4c3c2)c1 ; c1cc(-c2ccc(N(c3ccc(-c4cccc(-n5c6ccccc6c6ccccc65)c4)cc3)c3ccc(-c4ccc5c(c4)sc4c6ccccc6ccc54)cc3)cc2)cc(-c2ccc3c(ccc4ccccc43)c2)c1]

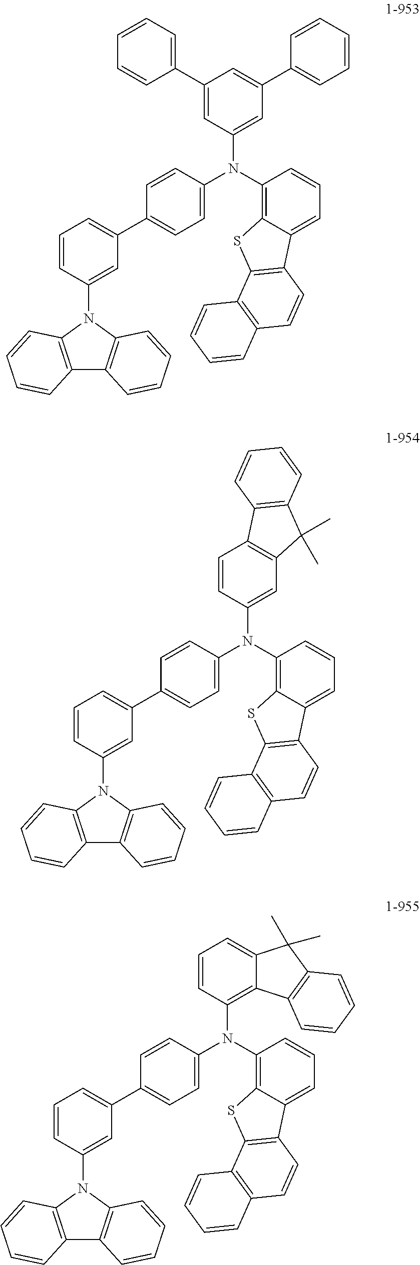 [SMILES: c1ccc(-c2cc(-c3ccccc3)cc(N(c3ccc(-c4cccc(-n5c6ccccc6c6ccccc65)c4)cc3)c3cccc4c3sc3c5ccccc5ccc43)c2)cc1 ; CC1(C)c2ccccc2-c2ccc(N(c3ccc(-c4cccc(-n5c6ccccc6c6ccccc65)c4)cc3)c3cccc4c3sc3c5ccccc5ccc43)cc21 ; CC1(C)c2ccccc2-c2c(N(c3ccc(-c4cccc(-n5c6ccccc6c6ccccc65)c4)cc3)c3cccc4c3sc3c5ccccc5ccc43)cccc21]

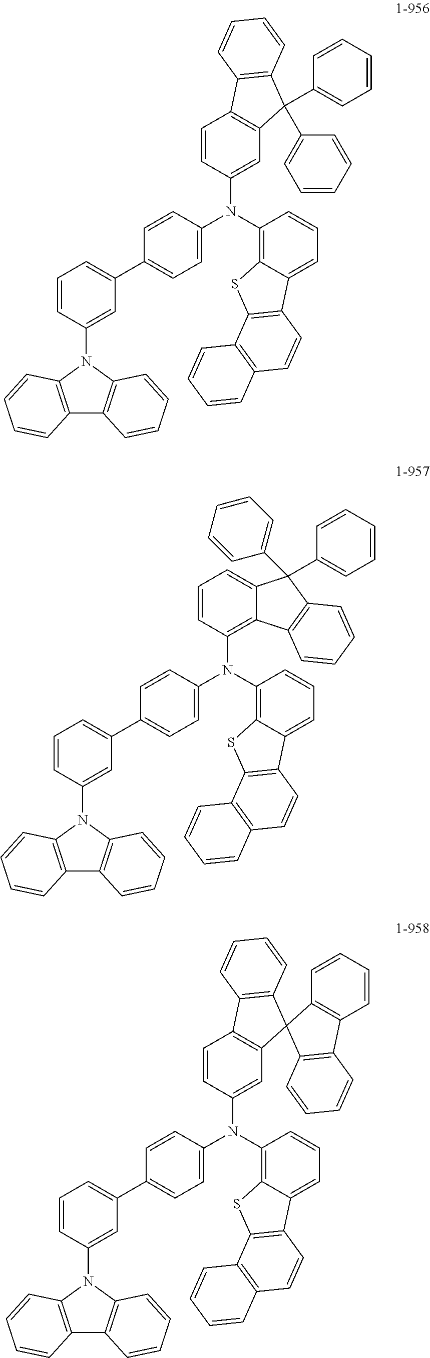 [SMILES: c1ccc(C2(c3ccccc3)c3ccccc3-c3ccc(N(c4ccc(-c5cccc(-n6c7ccccc7c7ccccc76)c5)cc4)c4cccc5c4sc4c6ccccc6ccc54)cc32)cc1 ; c1ccc(C2(c3ccccc3)c3ccccc3-c3c(N(c4ccc(-c5cccc(-n6c7ccccc7c7ccccc76)c5)cc4)c4cccc5c4sc4c6ccccc6ccc54)cccc32)cc1 ; c1cc(-c2ccc(N(c3ccc4c(c3)C3(c5ccccc5-c5ccccc53)c3ccccc3-4)c3cccc4c3sc3c5ccccc5ccc43)cc2)cc(-n2c3ccccc3c3ccccc32)c1]

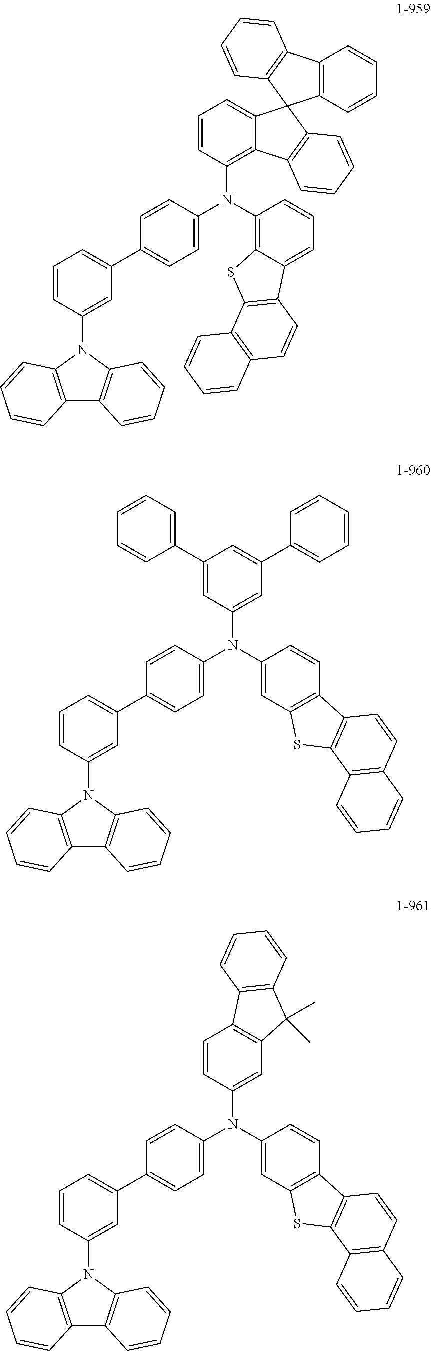 [SMILES: c1cc(-c2ccc(N(c3cccc4c3-c3ccccc3C43c4ccccc4-c4ccccc43)c3cccc4c3sc3c5ccccc5ccc43)cc2)cc(-n2c3ccccc3c3ccccc32)c1 ; c1ccc(-c2cc(-c3ccccc3)cc(N(c3ccc(-c4cccc(-n5c6ccccc6c6ccccc65)c4)cc3)c3ccc4c(c3)sc3c5ccccc5ccc43)c2)cc1 ; CC1(C)c2ccccc2-c2ccc(N(c3ccc(-c4cccc(-n5c6ccccc6c6ccccc65)c4)cc3)c3ccc4c(c3)sc3c5ccccc5ccc43)cc21]

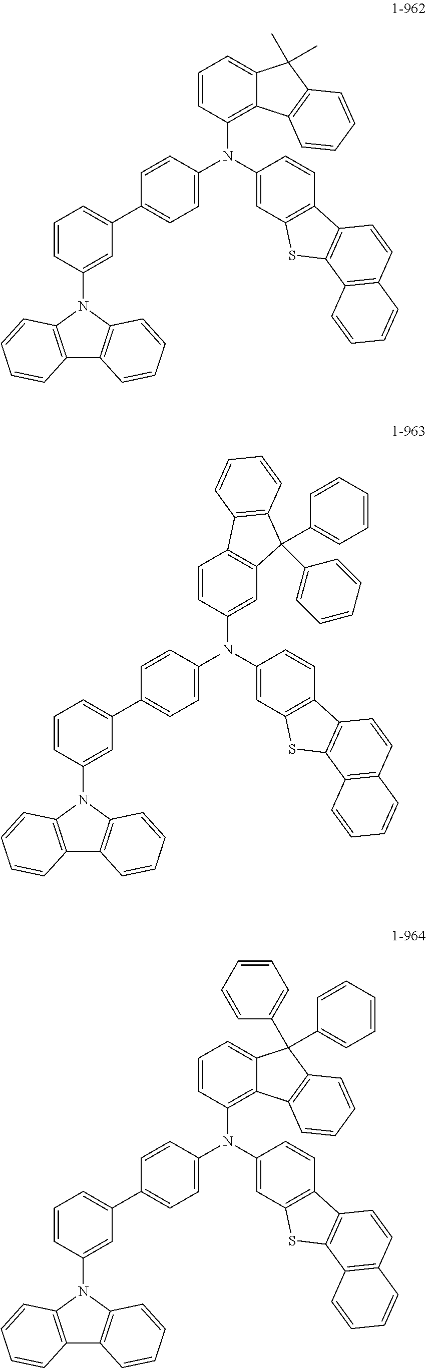 [SMILES: CC1(C)c2ccccc2-c2c(N(c3ccc(-c4cccc(-n5c6ccccc6c6ccccc65)c4)cc3)c3ccc4c(c3)sc3c5ccccc5ccc43)cccc21 ; c1ccc(C2(c3ccccc3)c3ccccc3-c3ccc(N(c4ccc(-c5cccc(-n6c7ccccc7c7ccccc76)c5)cc4)c4ccc5c(c4)sc4c6ccccc6ccc54)cc32)cc1 ; c1ccc(C2(c3ccccc3)c3ccccc3-c3c(N(c4ccc(-c5cccc(-n6c7ccccc7c7ccccc76)c5)cc4)c4ccc5c(c4)sc4c6ccccc6ccc54)cccc32)cc1]

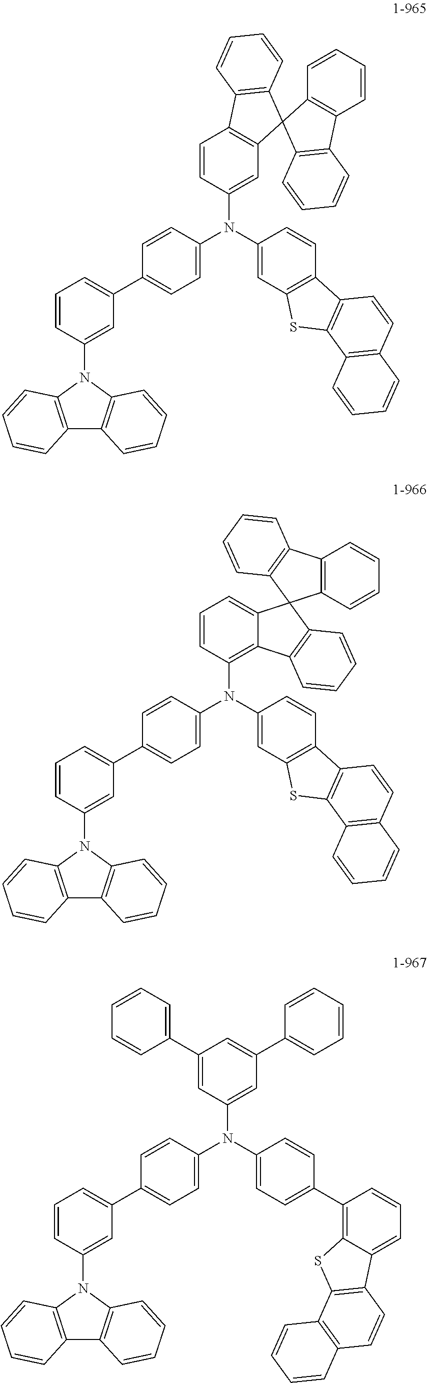 [SMILES: c1cc(-c2ccc(N(c3ccc4c(c3)C3(c5ccccc5-c5ccccc53)c3ccccc3-4)c3ccc4c(c3)sc3c5ccccc5ccc43)cc2)cc(-n2c3ccccc3c3ccccc32)c1 ; c1cc(-c2ccc(N(c3ccc4c(c3)sc3c5ccccc5ccc43)c3cccc4c3-c3ccccc3C43c4ccccc4-c4ccccc43)cc2)cc(-n2c3ccccc3c3ccccc32)c1 ; c1ccc(-c2cc(-c3ccccc3)cc(N(c3ccc(-c4cccc(-n5c6ccccc6c6ccccc65)c4)cc3)c3ccc(-c4cccc5c4sc4c6ccccc6ccc54)cc3)c2)cc1]

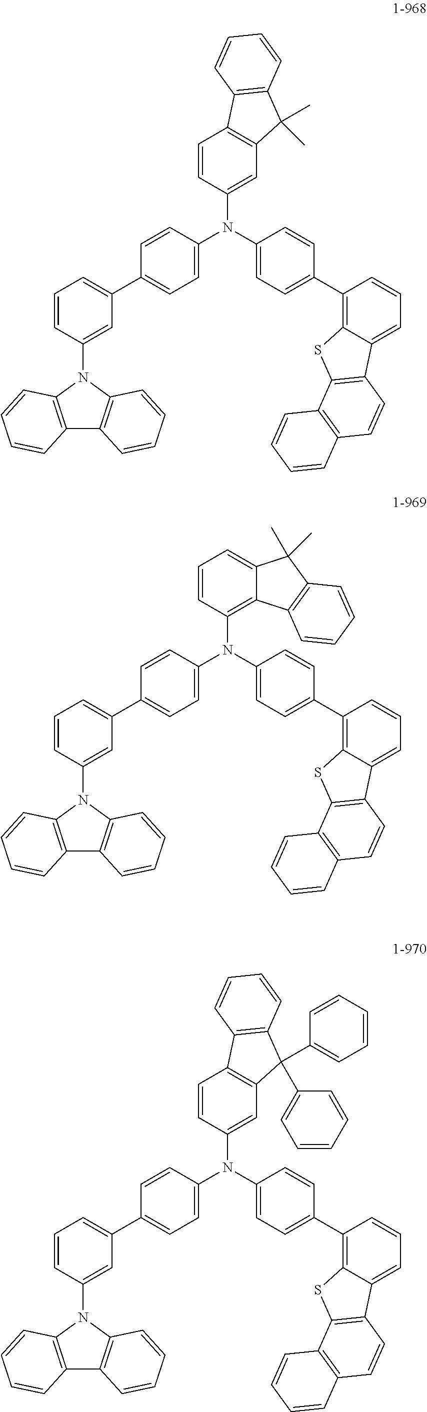 [SMILES: CC1(C)c2ccccc2-c2ccc(N(c3ccc(-c4cccc(-n5c6ccccc6c6ccccc65)c4)cc3)c3ccc(-c4cccc5c4sc4c6ccccc6ccc54)cc3)cc21 ; CC1(C)c2ccccc2-c2c(N(c3ccc(-c4cccc(-n5c6ccccc6c6ccccc65)c4)cc3)c3ccc(-c4cccc5c4sc4c6ccccc6ccc54)cc3)cccc21 ; c1ccc(C2(c3ccccc3)c3ccccc3-c3ccc(N(c4ccc(-c5cccc(-n6c7ccccc7c7ccccc76)c5)cc4)c4ccc(-c5cccc6c5sc5c7ccccc7ccc65)cc4)cc32)cc1]

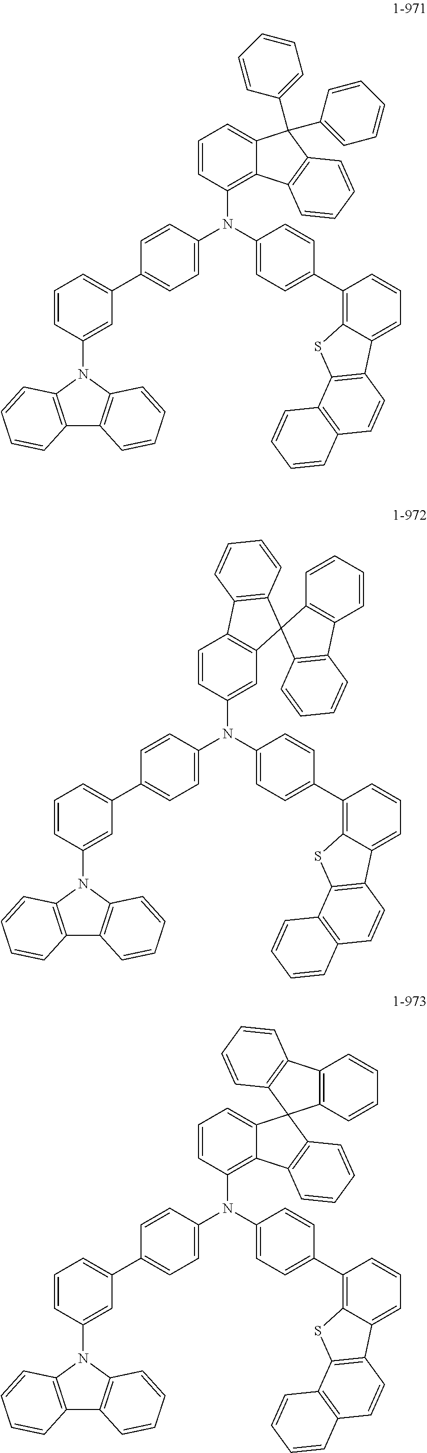 [SMILES: c1ccc(C2(c3ccccc3)c3ccccc3-c3c(N(c4ccc(-c5cccc(-n6c7ccccc7c7ccccc76)c5)cc4)c4ccc(-c5cccc6c5sc5c7ccccc7ccc65)cc4)cccc32)cc1 ; c1cc(-c2ccc(N(c3ccc(-c4cccc5c4sc4c6ccccc6ccc54)cc3)c3ccc4c(c3)C3(c5ccccc5-c5ccccc53)c3ccccc3-4)cc2)cc(-n2c3ccccc3c3ccccc32)c1 ; c1cc(-c2ccc(N(c3ccc(-c4cccc5c4sc4c6ccccc6ccc54)cc3)c3cccc4c3-c3ccccc3C43c4ccccc4-c4ccccc43)cc2)cc(-n2c3ccccc3c3ccccc32)c1]

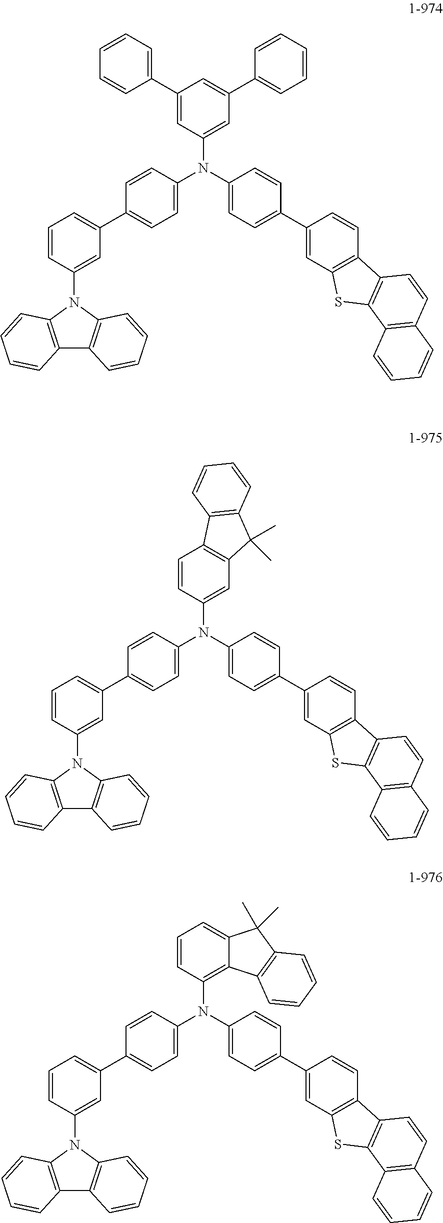 [SMILES: c1ccc(-c2cc(-c3ccccc3)cc(N(c3ccc(-c4cccc(-n5c6ccccc6c6ccccc65)c4)cc3)c3ccc(-c4ccc5c(c4)sc4c6ccccc6ccc54)cc3)c2)cc1 ; CC1(C)c2ccccc2-c2ccc(N(c3ccc(-c4cccc(-n5c6ccccc6c6ccccc65)c4)cc3)c3ccc(-c4ccc5c(c4)sc4c6ccccc6ccc54)cc3)cc21 ; CC1(C)c2ccccc2-c2c(N(c3ccc(-c4cccc(-n5c6ccccc6c6ccccc65)c4)cc3)c3ccc(-c4ccc5c(c4)sc4c6ccccc6ccc54)cc3)cccc21]

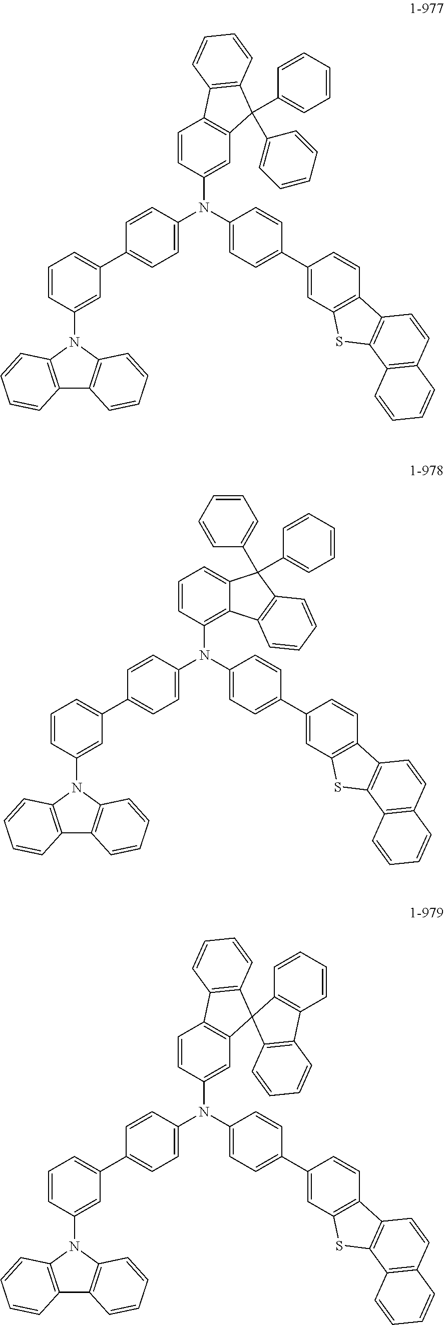 [SMILES: c1ccc(C2(c3ccccc3)c3ccccc3-c3ccc(N(c4ccc(-c5cccc(-n6c7ccccc7c7ccccc76)c5)cc4)c4ccc(-c5ccc6c(c5)sc5c7ccccc7ccc65)cc4)cc32)cc1 ; c1ccc(C2(c3ccccc3)c3ccccc3-c3c(N(c4ccc(-c5cccc(-n6c7ccccc7c7ccccc76)c5)cc4)c4ccc(-c5ccc6c(c5)sc5c7ccccc7ccc65)cc4)cccc32)cc1 ; c1cc(-c2ccc(N(c3ccc(-c4ccc5c(c4)sc4c6ccccc6ccc54)cc3)c3ccc4c(c3)C3(c5ccccc5-c5ccccc53)c3ccccc3-4)cc2)cc(-n2c3ccccc3c3ccccc32)c1]

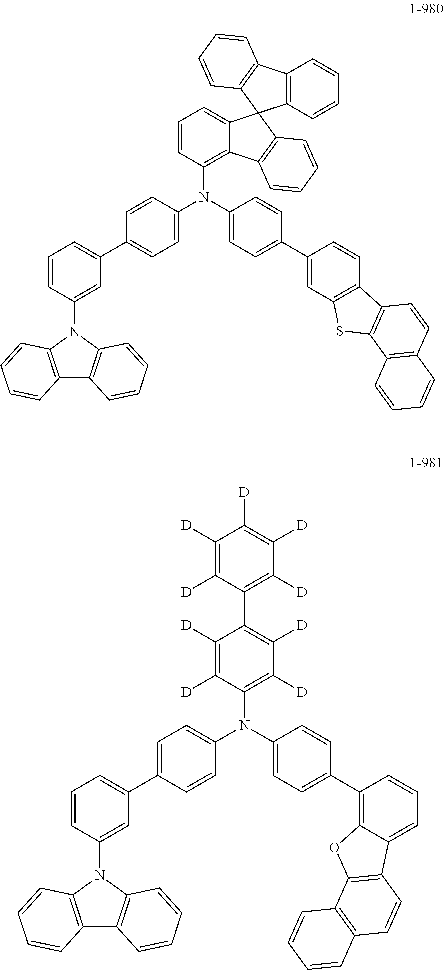 [SMILES: c1cc(-c2ccc(N(c3ccc(-c4ccc5c(c4)sc4c6ccccc6ccc54)cc3)c3cccc4c3-c3ccccc3C43c4ccccc4-c4ccccc43)cc2)cc(-n2c3ccccc3c3ccccc32)c1 ; [2H]c1c([2H])c([2H])c(-c2c([2H])c([2H])c(N(c3ccc(-c4cccc(-n5c6ccccc6c6ccccc65)c4)cc3)c3ccc(-c4cccc5c4oc4c6ccccc6ccc54)cc3)c([2H])c2[2H])c([2H])c1[2H]]

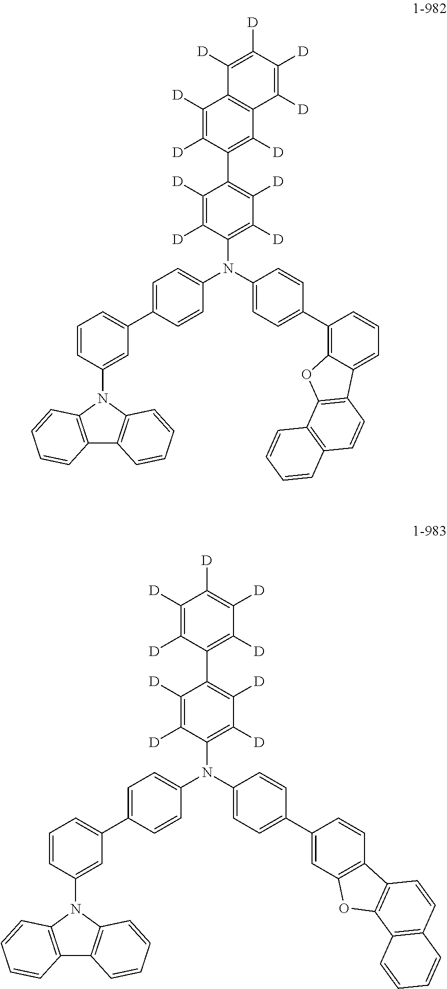 [SMILES: [2H]c1c([2H])c(N(c2ccc(-c3cccc(-n4c5ccccc5c5ccccc54)c3)cc2)c2ccc(-c3cccc4c3oc3c5ccccc5ccc43)cc2)c([2H])c([2H])c1-c1c([2H])c([2H])c2c([2H])c([2H])c([2H])c([2H])c2c1[2H] ; [2H]c1c([2H])c([2H])c(-c2c([2H])c([2H])c(N(c3ccc(-c4cccc(-n5c6ccccc6c6ccccc65)c4)cc3)c3ccc(-c4ccc5c(c4)oc4c6ccccc6ccc54)cc3)c([2H])c2[2H])c([2H])c1[2H]]

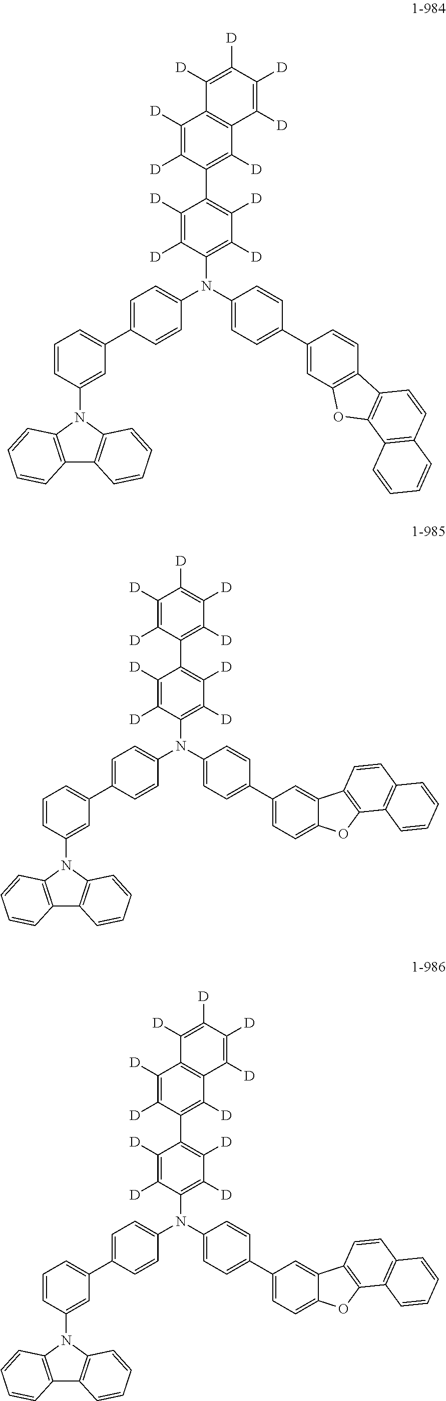 [SMILES: [2H]c1c([2H])c(N(c2ccc(-c3cccc(-n4c5ccccc5c5ccccc54)c3)cc2)c2ccc(-c3ccc4c(c3)oc3c5ccccc5ccc43)cc2)c([2H])c([2H])c1-c1c([2H])c([2H])c2c([2H])c([2H])c([2H])c([2H])c2c1[2H] ; [2H]c1c([2H])c([2H])c(-c2c([2H])c([2H])c(N(c3ccc(-c4cccc(-n5c6ccccc6c6ccccc65)c4)cc3)c3ccc(-c4ccc5oc6c7ccccc7ccc6c5c4)cc3)c([2H])c2[2H])c([2H])c1[2H] ; [2H]c1c([2H])c(N(c2ccc(-c3cccc(-n4c5ccccc5c5ccccc54)c3)cc2)c2ccc(-c3ccc4oc5c6ccccc6ccc5c4c3)cc2)c([2H])c([2H])c1-c1c([2H])c([2H])c2c([2H])c([2H])c([2H])c([2H])c2c1[2H]]

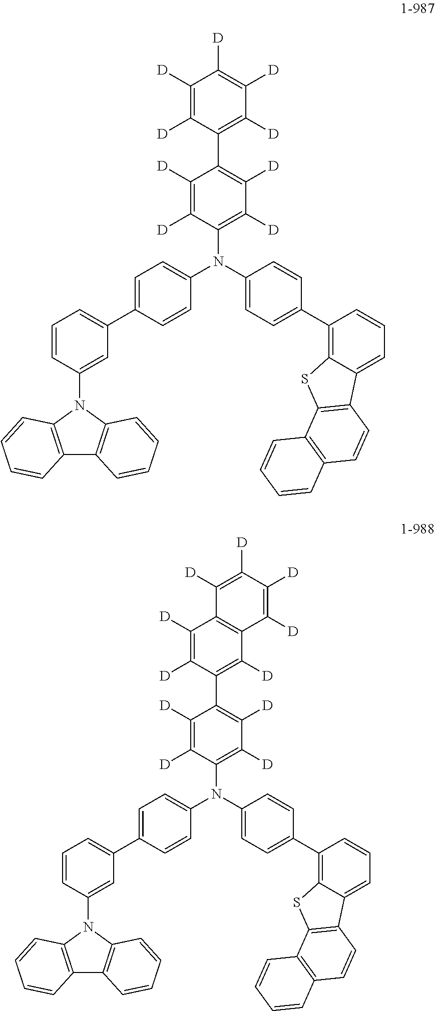 [SMILES: [2H]c1c([2H])c([2H])c(-c2c([2H])c([2H])c(N(c3ccc(-c4cccc(-n5c6ccccc6c6ccccc65)c4)cc3)c3ccc(-c4cccc5c4sc4c6ccccc6ccc54)cc3)c([2H])c2[2H])c([2H])c1[2H] ; [2H]c1c([2H])c(N(c2ccc(-c3cccc(-n4c5ccccc5c5ccccc54)c3)cc2)c2ccc(-c3cccc4c3sc3c5ccccc5ccc43)cc2)c([2H])c([2H])c1-c1c([2H])c([2H])c2c([2H])c([2H])c([2H])c([2H])c2c1[2H]]

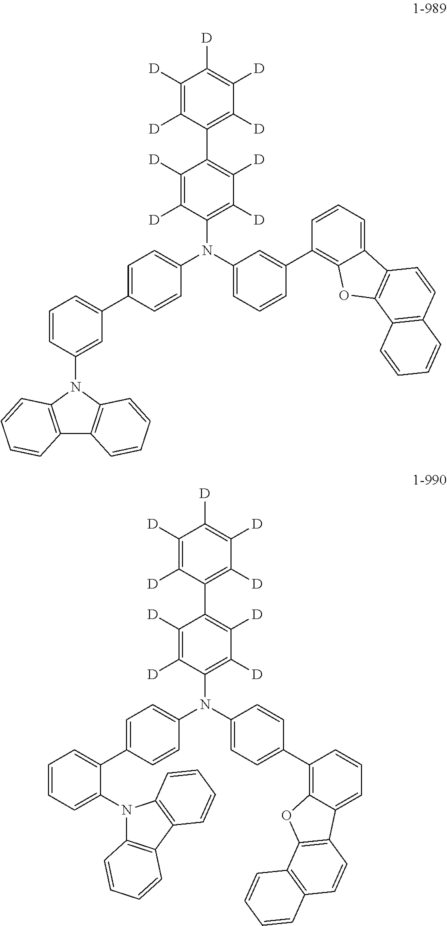 [SMILES: [2H]c1c([2H])c([2H])c(-c2c([2H])c([2H])c(N(c3ccc(-c4cccc(-n5c6ccccc6c6ccccc65)c4)cc3)c3cccc(-c4cccc5c4oc4c6ccccc6ccc54)c3)c([2H])c2[2H])c([2H])c1[2H] ; [2H]c1c([2H])c([2H])c(-c2c([2H])c([2H])c(N(c3ccc(-c4ccccc4-n4c5ccccc5c5ccccc54)cc3)c3ccc(-c4cccc5c4oc4c6ccccc6ccc54)cc3)c([2H])c2[2H])c([2H])c1[2H]]

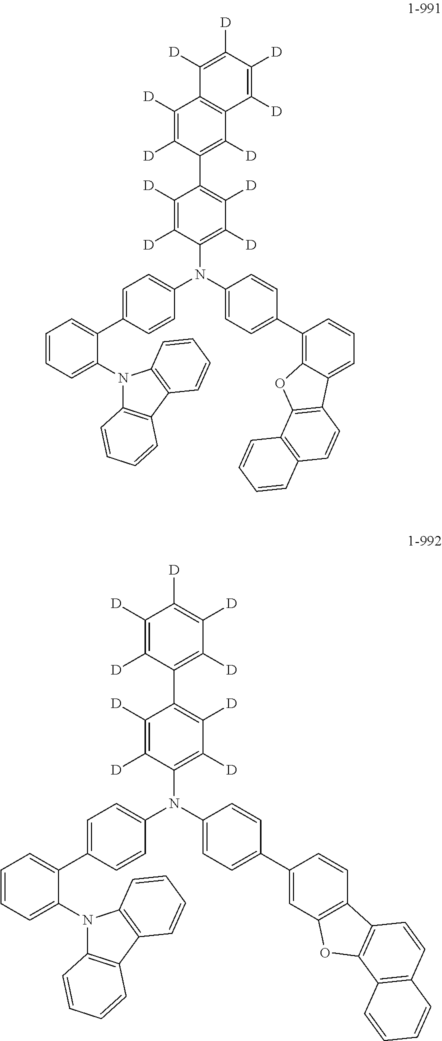 [SMILES: [2H]c1c([2H])c(N(c2ccc(-c3ccccc3-n3c4ccccc4c4ccccc43)cc2)c2ccc(-c3cccc4c3oc3c5ccccc5ccc43)cc2)c([2H])c([2H])c1-c1c([2H])c([2H])c2c([2H])c([2H])c([2H])c([2H])c2c1[2H] ; [2H]c1c([2H])c([2H])c(-c2c([2H])c([2H])c(N(c3ccc(-c4ccc5c(c4)oc4c6ccccc6ccc54)cc3)c3ccc(-c4ccccc4-n4c5ccccc5c5ccccc54)cc3)c([2H])c2[2H])c([2H])c1[2H]]

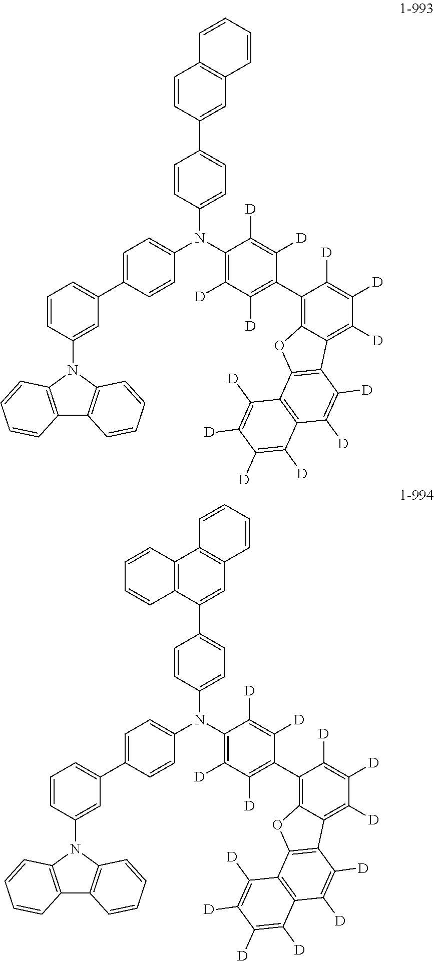

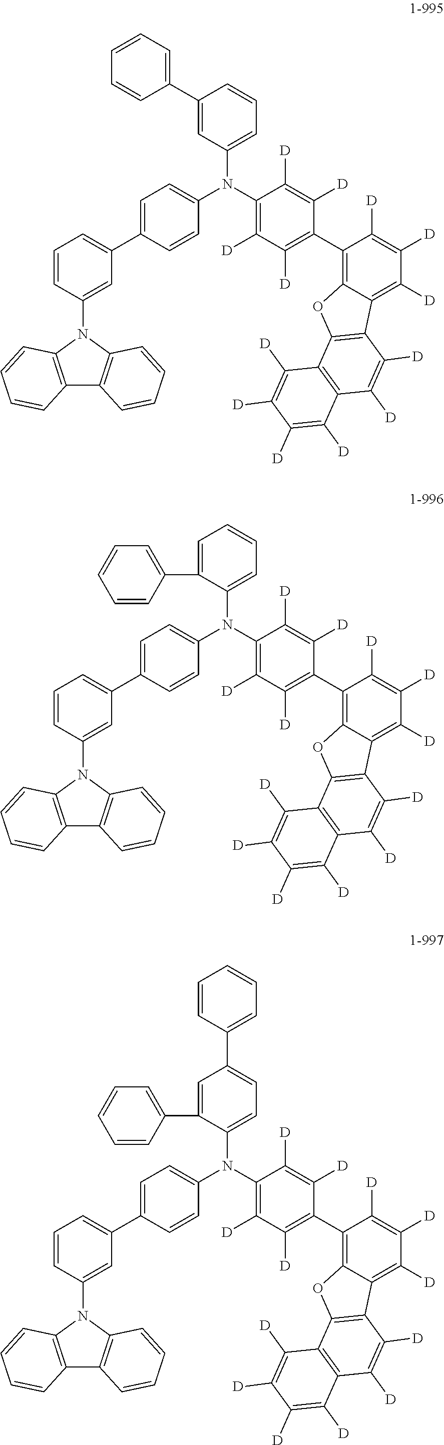

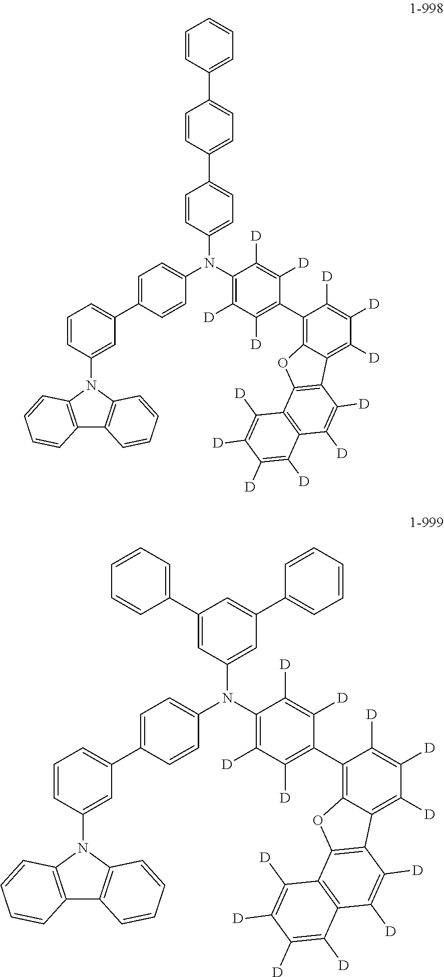 [SMILES: [2H]c1c([2H])c(N(c2ccc(-c3ccc(-c4ccccc4)cc3)cc2)c2ccc(-c3cccc(-n4c5ccccc5c5ccccc54)c3)cc2)c([2H])c([2H])c1-c1c([2H])c([2H])c([2H])c2c1oc1c3c([2H])c([2H])c([2H])c([2H])c3c([2H])c([2H])c12 ; [2H]c1c([2H])c(N(c2ccc(-c3cccc(-n4c5ccccc5c5ccccc54)c3)cc2)c2cc(-c3ccccc3)cc(-c3ccccc3)c2)c([2H])c([2H])c1-c1c([2H])c([2H])c([2H])c2c1oc1c3c([2H])c([2H])c([2H])c([2H])c3c([2H])c([2H])c12]

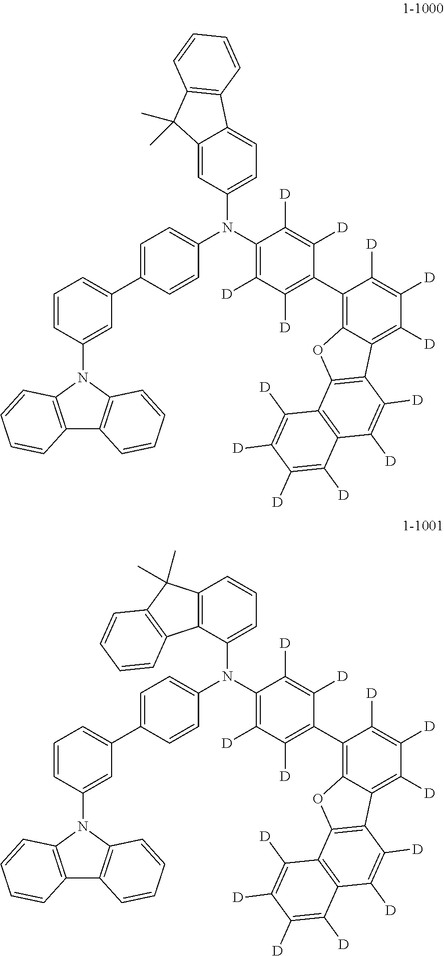 [SMILES: [2H]c1c([2H])c(N(c2ccc(-c3cccc(-n4c5ccccc5c5ccccc54)c3)cc2)c2ccc3c(c2)C(C)(C)c2ccccc2-3)c([2H])c([2H])c1-c1c([2H])c([2H])c([2H])c2c1oc1c3c([2H])c([2H])c([2H])c([2H])c3c([2H])c([2H])c12 ; [2H]c1c([2H])c(N(c2ccc(-c3cccc(-n4c5ccccc5c5ccccc54)c3)cc2)c2cccc3c2-c2ccccc2C3(C)C)c([2H])c([2H])c1-c1c([2H])c([2H])c([2H])c2c1oc1c3c([2H])c([2H])c([2H])c([2H])c3c([2H])c([2H])c12]

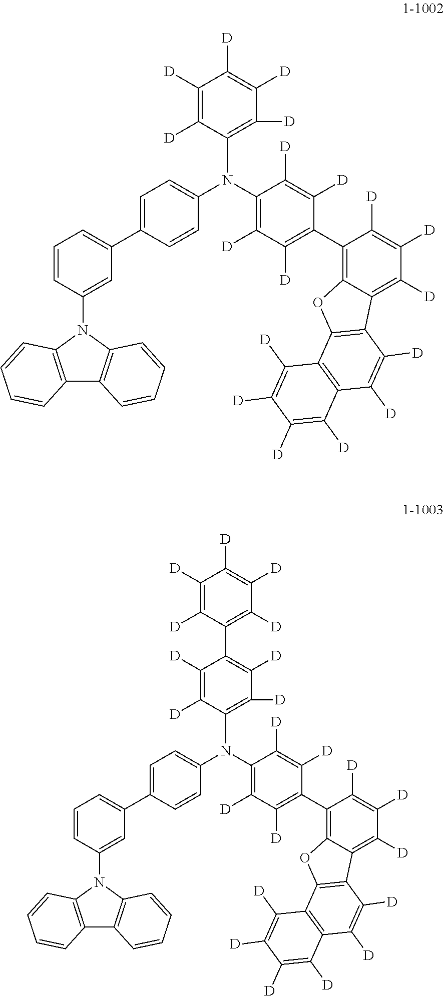 [SMILES: [2H]c1c([2H])c([2H])c(N(c2ccc(-c3cccc(-n4c5ccccc5c5ccccc54)c3)cc2)c2c([2H])c([2H])c(-c3c([2H])c([2H])c([2H])c4c3oc3c5c([2H])c([2H])c([2H])c([2H])c5c([2H])c([2H])c34)c([2H])c2[2H])c([2H])c1[2H] ; [2H]c1c([2H])c([2H])c(-c2c([2H])c([2H])c(N(c3ccc(-c4cccc(-n5c6ccccc6c6ccccc65)c4)cc3)c3c([2H])c([2H])c(-c4c([2H])c([2H])c([2H])c5c4oc4c6c([2H])c([2H])c([2H])c([2H])c6c([2H])c([2H])c45)c([2H])c3[2H])c([2H])c2[2H])c([2H])c1[2H]]

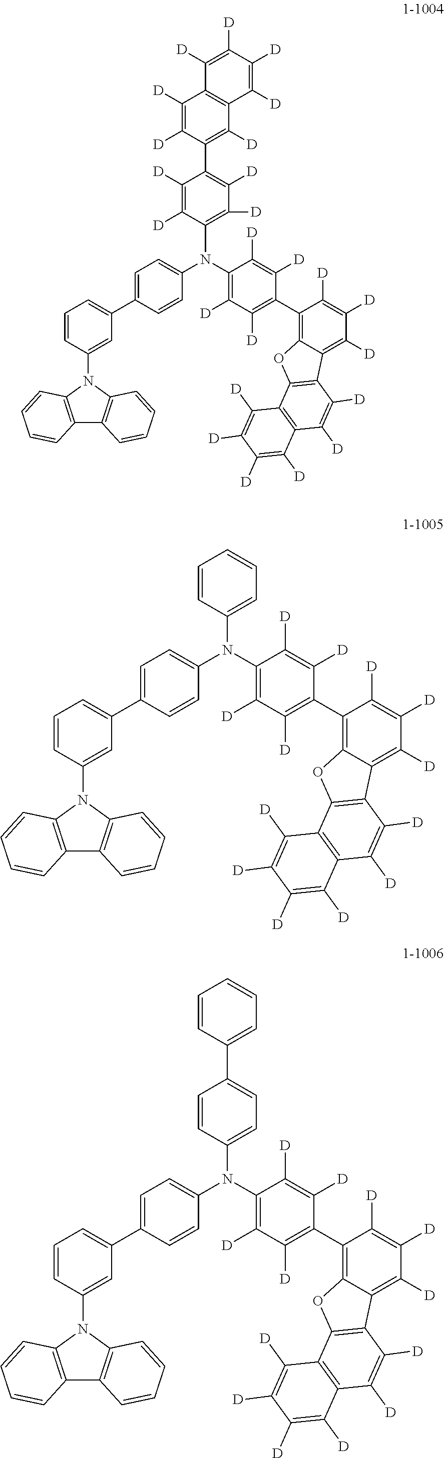

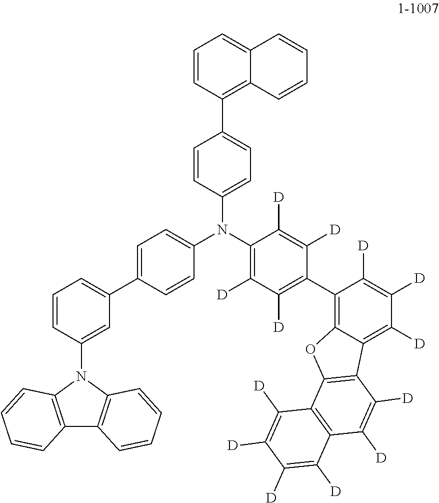 [SMILES: [2H]c1c([2H])c(N(c2ccc(-c3cccc(-n4c5ccccc5c5ccccc54)c3)cc2)c2ccc(-c3cccc4ccccc34)cc2)c([2H])c([2H])c1-c1c([2H])c([2H])c([2H])c2c1oc1c3c([2H])c([2H])c([2H])c([2H])c3c([2H])c([2H])c12]

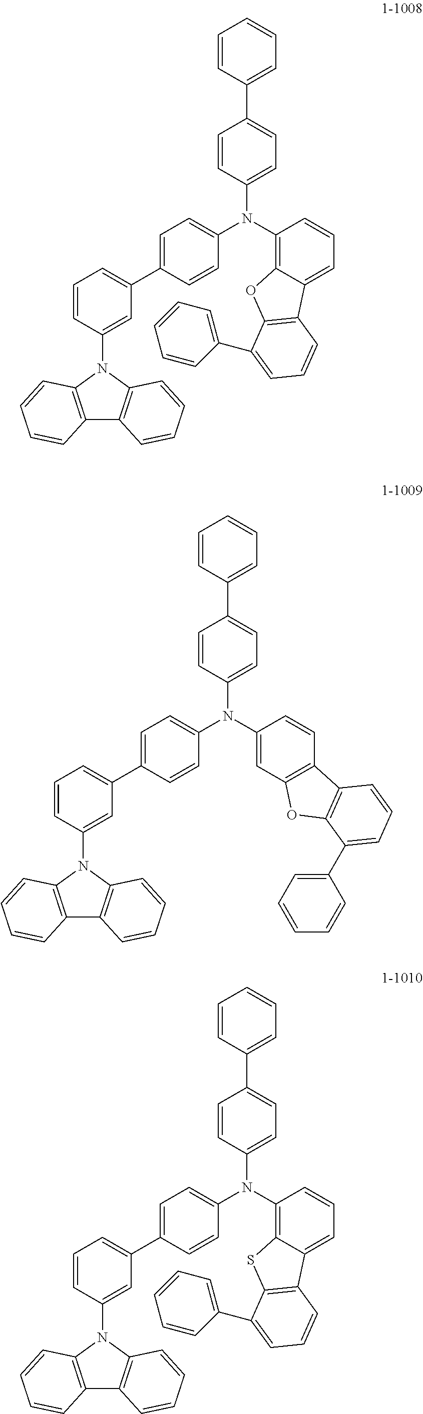 [SMILES: c1ccc(-c2ccc(N(c3ccc(-c4cccc(-n5c6ccccc6c6ccccc65)c4)cc3)c3cccc4c3oc3c(-c5ccccc5)cccc34)cc2)cc1 ; c1ccc(-c2ccc(N(c3ccc(-c4cccc(-n5c6ccccc6c6ccccc65)c4)cc3)c3ccc4c(c3)oc3c(-c5ccccc5)cccc34)cc2)cc1 ; c1ccc(-c2ccc(N(c3ccc(-c4cccc(-n5c6ccccc6c6ccccc65)c4)cc3)c3cccc4c3sc3c(-c5ccccc5)cccc34)cc2)cc1]

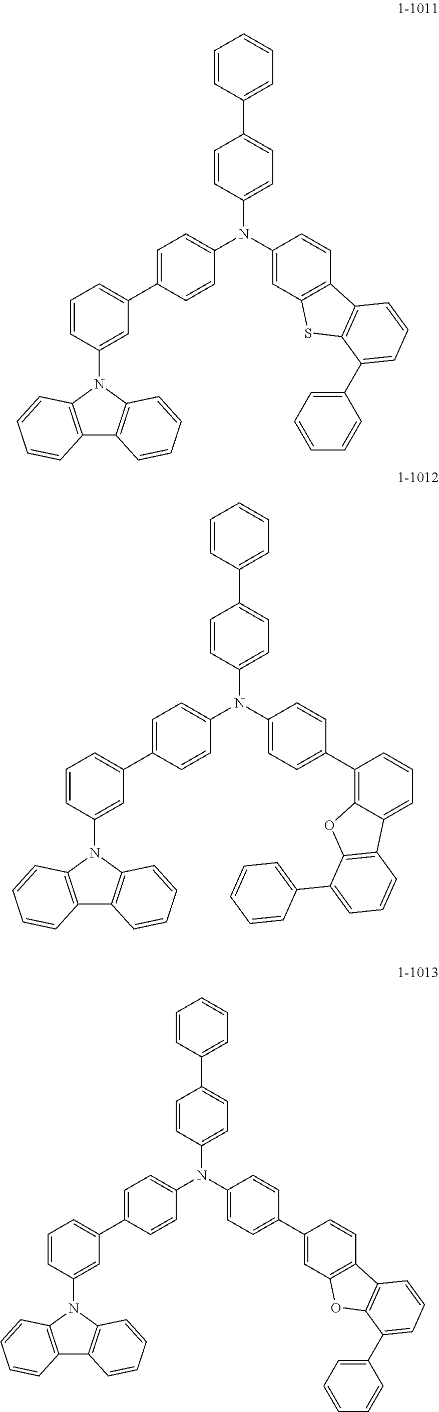 [SMILES: c1ccc(-c2ccc(N(c3ccc(-c4cccc(-n5c6ccccc6c6ccccc65)c4)cc3)c3ccc4c(c3)sc3c(-c5ccccc5)cccc34)cc2)cc1 ; c1ccc(-c2ccc(N(c3ccc(-c4cccc(-n5c6ccccc6c6ccccc65)c4)cc3)c3ccc(-c4cccc5c4oc4c(-c6ccccc6)cccc45)cc3)cc2)cc1 ; c1ccc(-c2ccc(N(c3ccc(-c4cccc(-n5c6ccccc6c6ccccc65)c4)cc3)c3ccc(-c4ccc5c(c4)oc4c(-c6ccccc6)cccc45)cc3)cc2)cc1]

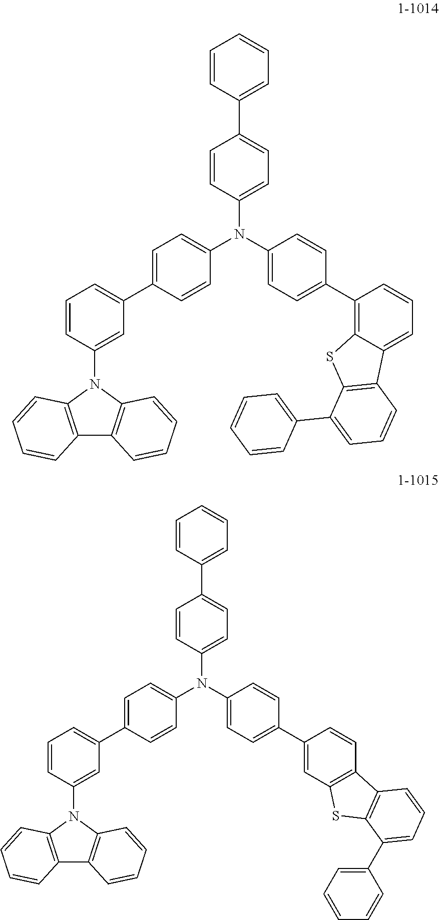 [SMILES: c1ccc(-c2ccc(N(c3ccc(-c4cccc(-n5c6ccccc6c6ccccc65)c4)cc3)c3ccc(-c4cccc5c4sc4c(-c6ccccc6)cccc45)cc3)cc2)cc1 ; c1ccc(-c2ccc(N(c3ccc(-c4cccc(-n5c6ccccc6c6ccccc65)c4)cc3)c3ccc(-c4ccc5c(c4)sc4c(-c6ccccc6)cccc45)cc3)cc2)cc1]

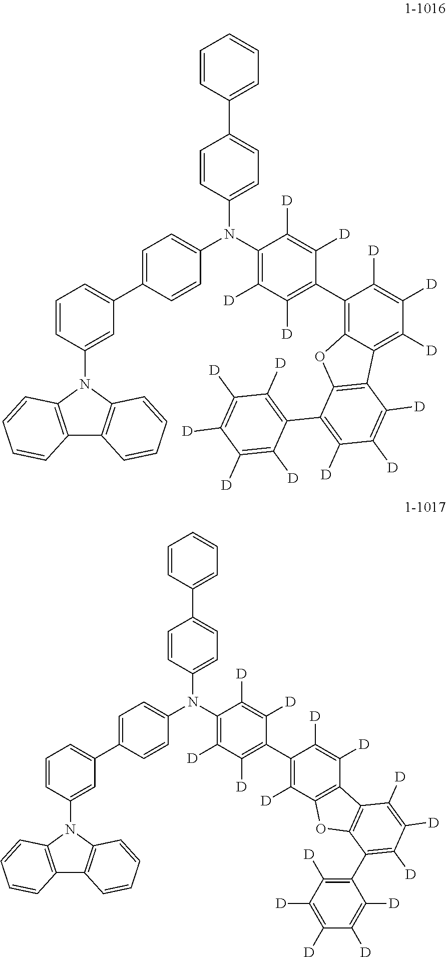 [SMILES: [2H]c1c([2H])c([2H])c(-c2c([2H])c([2H])c([2H])c3c2oc2c(-c4c([2H])c([2H])c(N(c5ccc(-c6ccccc6)cc5)c5ccc(-c6cccc(-n7c8ccccc8c8ccccc87)c6)cc5)c([2H])c4[2H])c([2H])c([2H])c([2H])c23)c([2H])c1[2H] ; [2H]c1c([2H])c([2H])c(-c2c([2H])c([2H])c([2H])c3c2oc2c([2H])c(-c4c([2H])c([2H])c(N(c5ccc(-c6ccccc6)cc5)c5ccc(-c6cccc(-n7c8ccccc8c8ccccc87)c6)cc5)c([2H])c4[2H])c([2H])c([2H])c23)c([2H])c1[2H]]

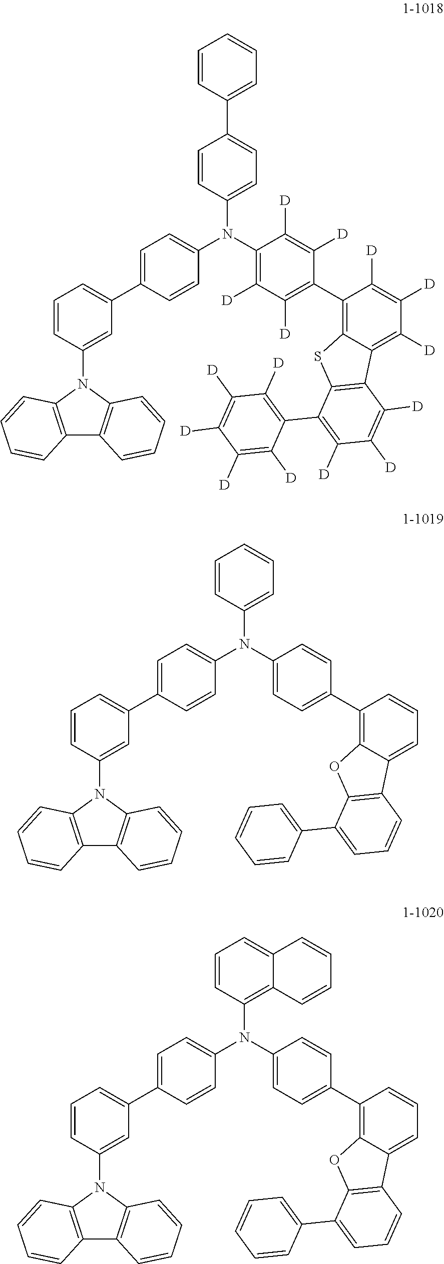 [SMILES: [2H]c1c([2H])c([2H])c(-c2c([2H])c([2H])c([2H])c3c2sc2c(-c4c([2H])c([2H])c(N(c5ccc(-c6ccccc6)cc5)c5ccc(-c6cccc(-n7c8ccccc8c8ccccc87)c6)cc5)c([2H])c4[2H])c([2H])c([2H])c([2H])c23)c([2H])c1[2H] ; c1ccc(-c2cccc3c2oc2c(-c4ccc(N(c5ccccc5)c5ccc(-c6cccc(-n7c8ccccc8c8ccccc87)c6)cc5)cc4)cccc23)cc1 ; c1ccc(-c2cccc3c2oc2c(-c4ccc(N(c5ccc(-c6cccc(-n7c8ccccc8c8ccccc87)c6)cc5)c5cccc6ccccc56)cc4)cccc23)cc1]

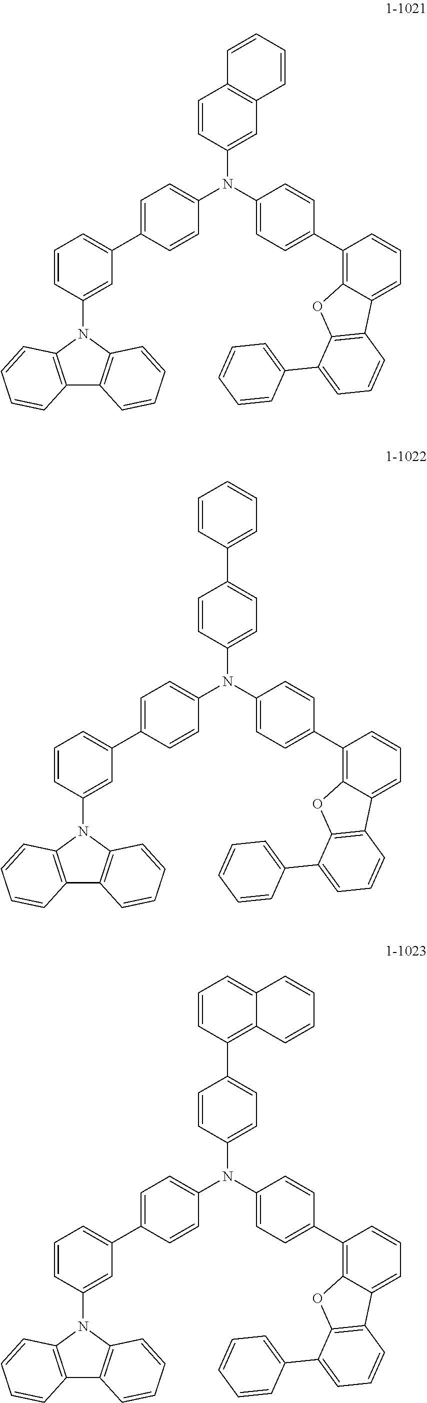 [SMILES: c1ccc(-c2cccc3c2oc2c(-c4ccc(N(c5ccc(-c6cccc(-n7c8ccccc8c8ccccc87)c6)cc5)c5ccc6ccccc6c5)cc4)cccc23)cc1 ; c1ccc(-c2ccc(N(c3ccc(-c4cccc(-n5c6ccccc6c6ccccc65)c4)cc3)c3ccc(-c4cccc5c4oc4c(-c6ccccc6)cccc45)cc3)cc2)cc1 ; c1ccc(-c2cccc3c2oc2c(-c4ccc(N(c5ccc(-c6cccc(-n7c8ccccc8c8ccccc87)c6)cc5)c5ccc(-c6cccc7ccccc67)cc5)cc4)cccc23)cc1]

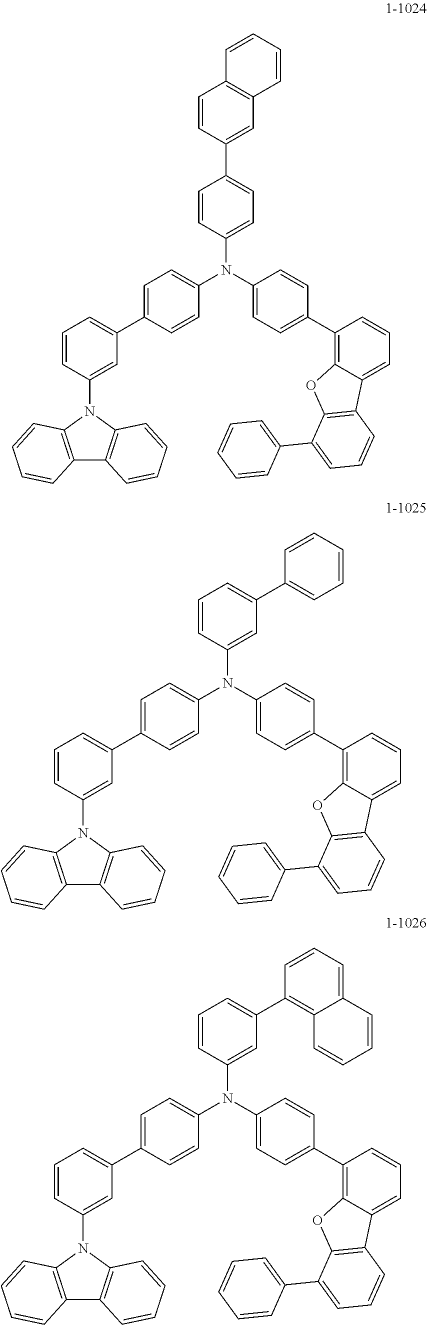 [SMILES: c1ccc(-c2cccc3c2oc2c(-c4ccc(N(c5ccc(-c6cccc(-n7c8ccccc8c8ccccc87)c6)cc5)c5ccc(-c6ccc7ccccc7c6)cc5)cc4)cccc23)cc1 ; c1ccc(-c2cccc(N(c3ccc(-c4cccc(-n5c6ccccc6c6ccccc65)c4)cc3)c3ccc(-c4cccc5c4oc4c(-c6ccccc6)cccc45)cc3)c2)cc1 ; c1ccc(-c2cccc3c2oc2c(-c4ccc(N(c5ccc(-c6cccc(-n7c8ccccc8c8ccccc87)c6)cc5)c5cccc(-c6cccc7ccccc67)c5)cc4)cccc23)cc1]

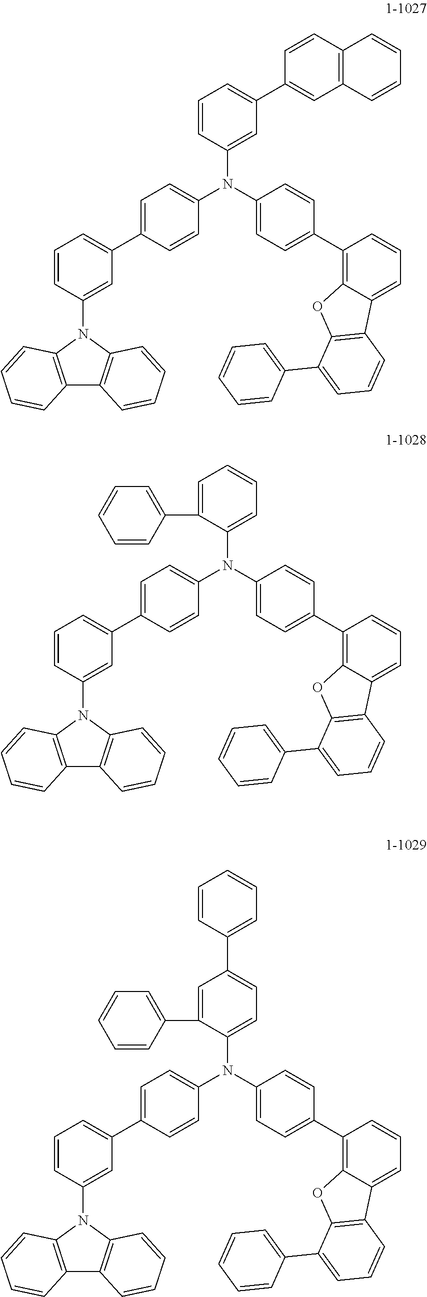 [SMILES: c1ccc(-c2cccc3c2oc2c(-c4ccc(N(c5ccc(-c6cccc(-n7c8ccccc8c8ccccc87)c6)cc5)c5cccc(-c6ccc7ccccc7c6)c5)cc4)cccc23)cc1 ; c1ccc(-c2ccccc2N(c2ccc(-c3cccc(-n4c5ccccc5c5ccccc54)c3)cc2)c2ccc(-c3cccc4c3oc3c(-c5ccccc5)cccc34)cc2)cc1 ; c1ccc(-c2ccc(N(c3ccc(-c4cccc(-n5c6ccccc6c6ccccc65)c4)cc3)c3ccc(-c4cccc5c4oc4c(-c6ccccc6)cccc45)cc3)c(-c3ccccc3)c2)cc1]

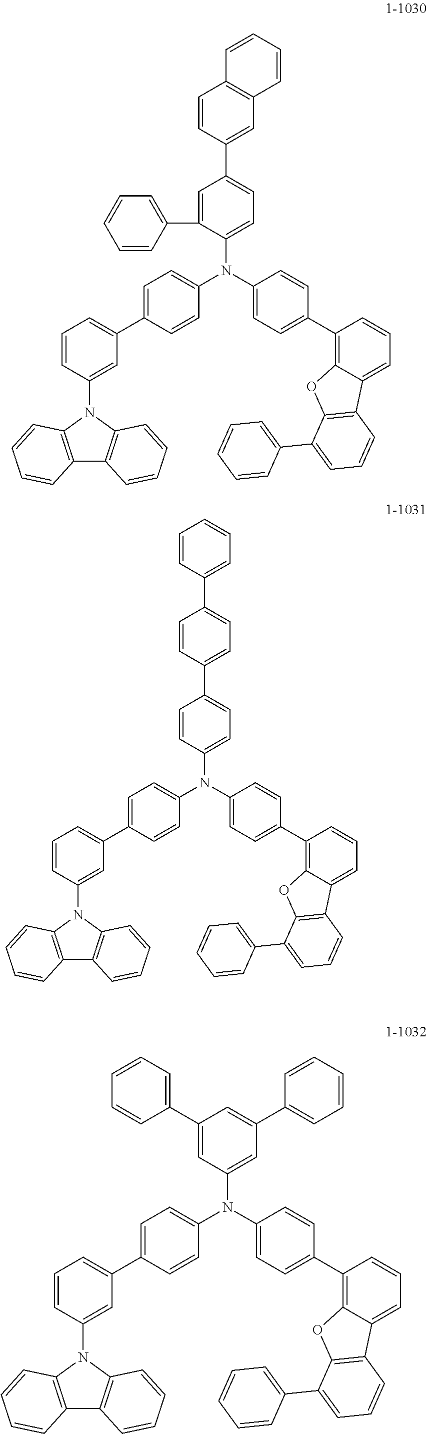 [SMILES: c1ccc(-c2cc(-c3ccc4ccccc4c3)ccc2N(c2ccc(-c3cccc(-n4c5ccccc5c5ccccc54)c3)cc2)c2ccc(-c3cccc4c3oc3c(-c5ccccc5)cccc34)cc2)cc1 ; c1ccc(-c2ccc(-c3ccc(N(c4ccc(-c5cccc(-n6c7ccccc7c7ccccc76)c5)cc4)c4ccc(-c5cccc6c5oc5c(-c7ccccc7)cccc56)cc4)cc3)cc2)cc1 ; c1ccc(-c2cc(-c3ccccc3)cc(N(c3ccc(-c4cccc(-n5c6ccccc6c6ccccc65)c4)cc3)c3ccc(-c4cccc5c4oc4c(-c6ccccc6)cccc45)cc3)c2)cc1]

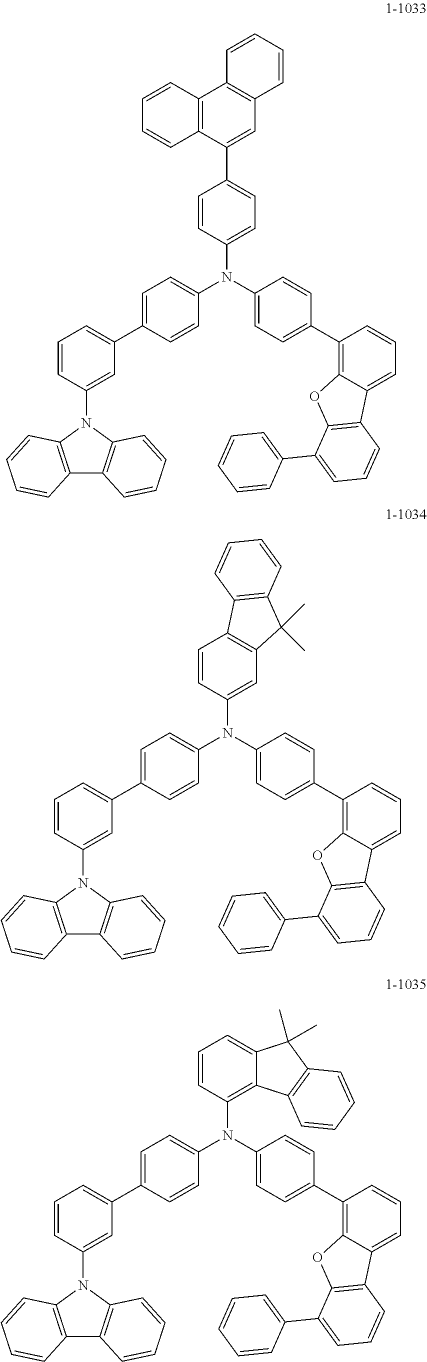 [SMILES: c1ccc(-c2cccc3c2oc2c(-c4ccc(N(c5ccc(-c6cccc(-n7c8ccccc8c8ccccc87)c6)cc5)c5ccc(-c6cc7ccccc7c7ccccc67)cc5)cc4)cccc23)cc1 ; CC1(C)c2ccccc2-c2ccc(N(c3ccc(-c4cccc(-n5c6ccccc6c6ccccc65)c4)cc3)c3ccc(-c4cccc5c4oc4c(-c6ccccc6)cccc45)cc3)cc21 ; CC1(C)c2ccccc2-c2c(N(c3ccc(-c4cccc(-n5c6ccccc6c6ccccc65)c4)cc3)c3ccc(-c4cccc5c4oc4c(-c6ccccc6)cccc45)cc3)cccc21]

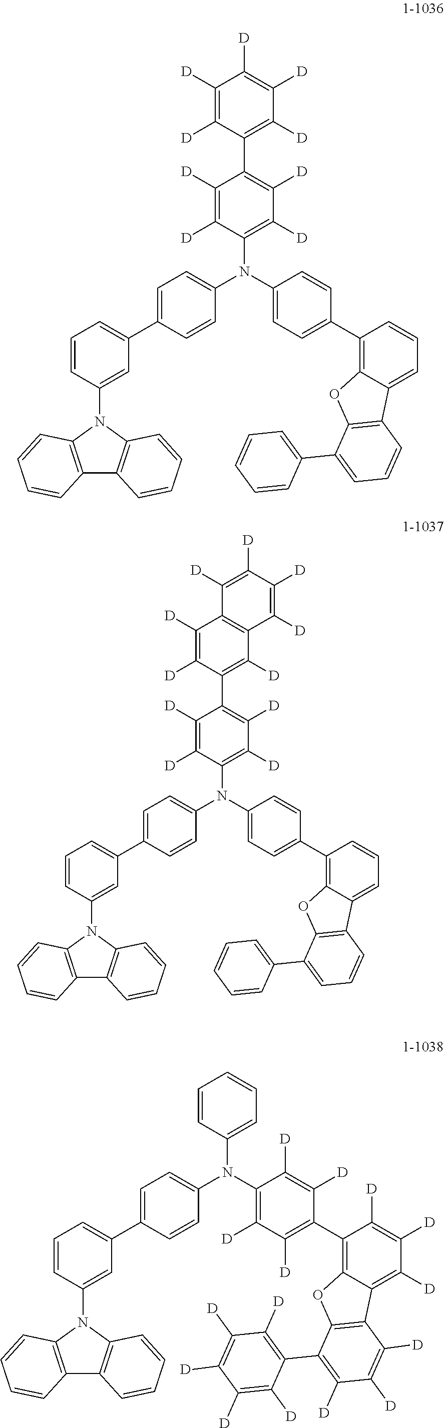

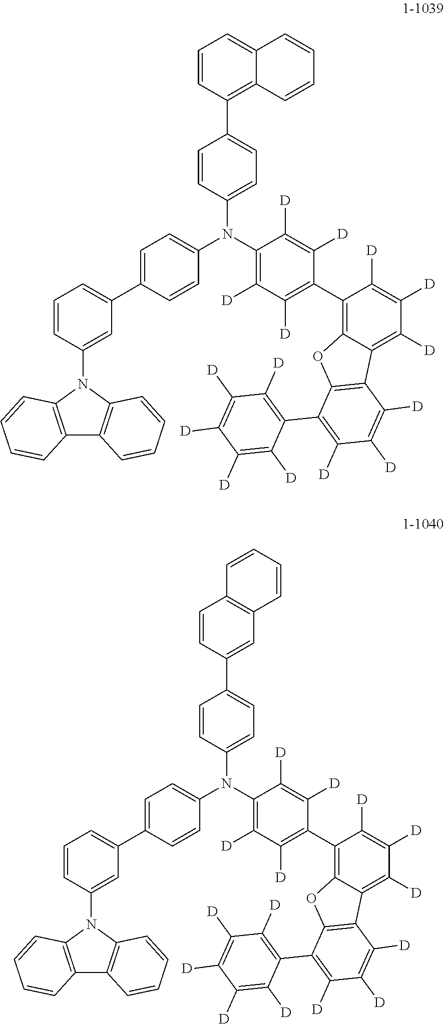 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5ccc(-c6cccc7ccccc67)cc5)c([2H])c4[2H])c([2H])c([2H])c([2H])c23)c([2H])c1[2H] ; [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5ccc(-c6ccc7ccccc7c6)cc5)c([2H])c4[2H])c([2H])c([2H])c([2H])c23)c([2H])c1[2H]]

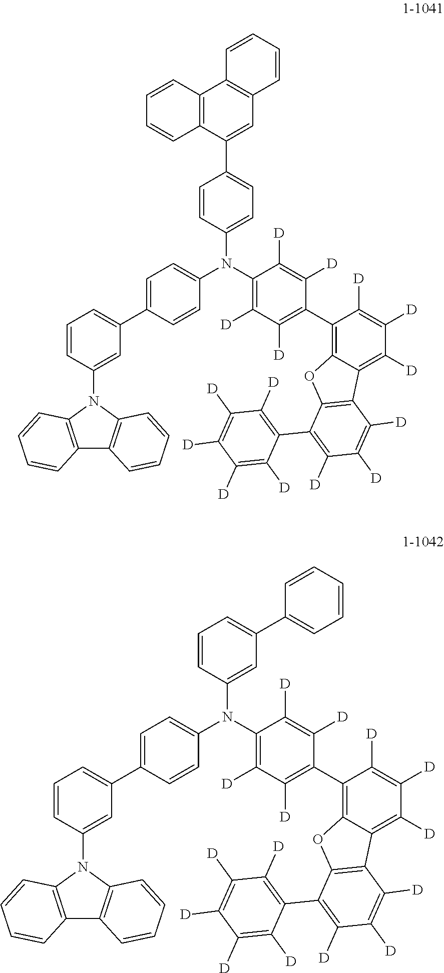 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5ccc(-c6cc7ccccc7c7ccccc67)cc5)c([2H])c4[2H])c([2H])c([2H])c([2H])c23)c([2H])c1[2H] ; [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5cccc(-c6ccccc6)c5)c([2H])c4[2H])c([2H])c([2H])c([2H])c23)c([2H])c1[2H]]

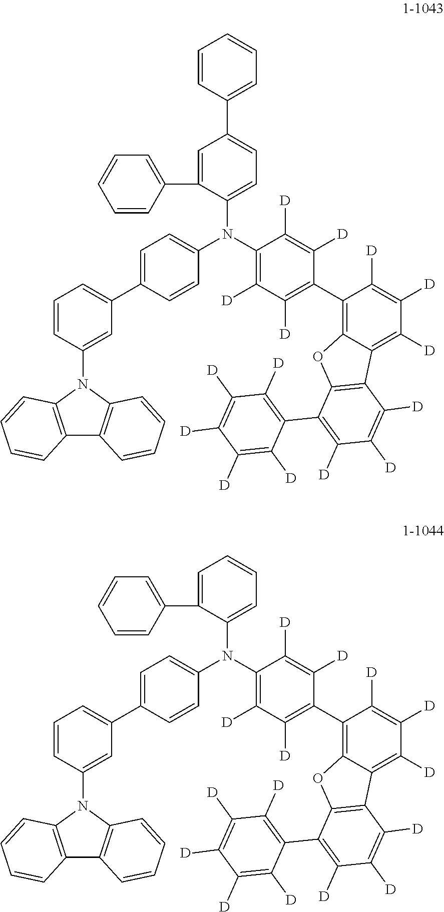 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5ccc(-c6ccccc6)cc5-c5ccccc5)c([2H])c4[2H])c([2H])c([2H])c([2H])c23)c([2H])c1[2H] ; [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5ccccc5-c5ccccc5)c([2H])c4[2H])c([2H])c([2H])c([2H])c23)c([2H])c1[2H]]

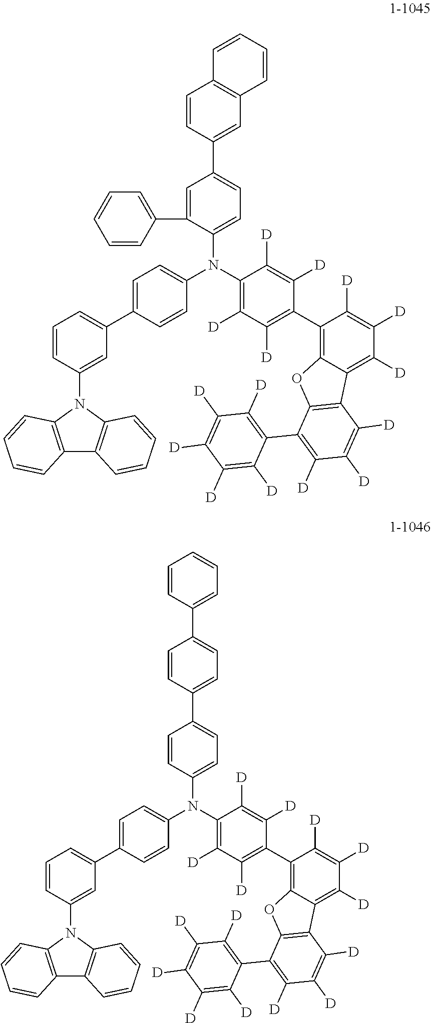 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5ccc(-c6ccc7ccccc7c6)cc5-c5ccccc5)c([2H])c4[2H])c([2H])c([2H])c([2H])c23)c([2H])c1[2H] ; [2H]c1c([2H])c([2H])c(-c2c([2H])c([2H])c([2H])c3c2oc2c(-c4c([2H])c([2H])c(N(c5ccc(-c6ccc(-c7ccccc7)cc6)cc5)c5ccc(-c6cccc(-n7c8ccccc8c8ccccc87)c6)cc5)c([2H])c4[2H])c([2H])c([2H])c([2H])c23)c([2H])c1[2H]]

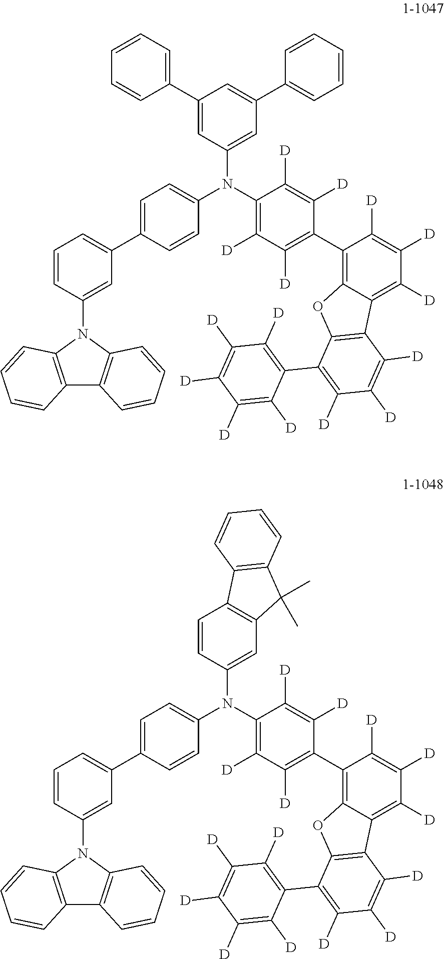 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5cc(-c6ccccc6)cc(-c6ccccc6)c5)c([2H])c4[2H])c([2H])c([2H])c([2H])c23)c([2H])c1[2H] ; [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5ccc6c(c5)C(C)(C)c5ccccc5-6)c([2H])c4[2H])c([2H])c([2H])c([2H])c23)c([2H])c1[2H]]

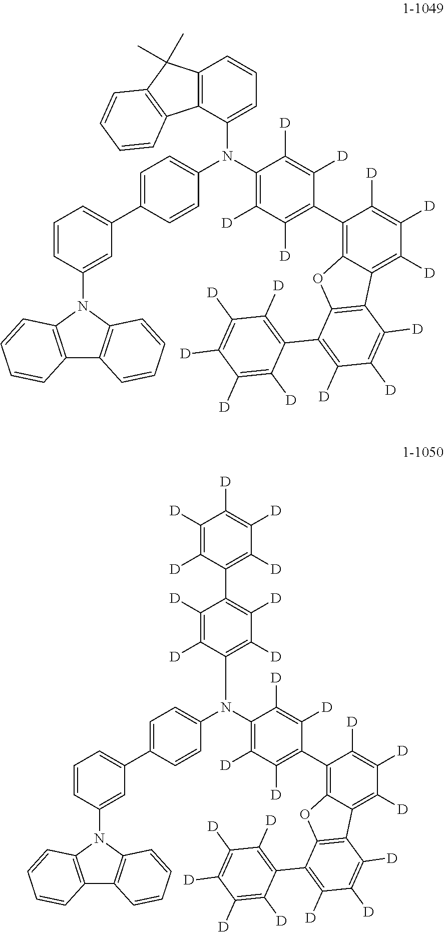 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5cccc6c5-c5ccccc5C6(C)C)c([2H])c4[2H])c([2H])c([2H])c([2H])c23)c([2H])c1[2H] ; [2H]c1c([2H])c([2H])c(-c2c([2H])c([2H])c(N(c3ccc(-c4cccc(-n5c6ccccc6c6ccccc65)c4)cc3)c3c([2H])c([2H])c(-c4c([2H])c([2H])c([2H])c5c4oc4c(-c6c([2H])c([2H])c([2H])c([2H])c6[2H])c([2H])c([2H])c([2H])c45)c([2H])c3[2H])c([2H])c2[2H])c([2H])c1[2H]]

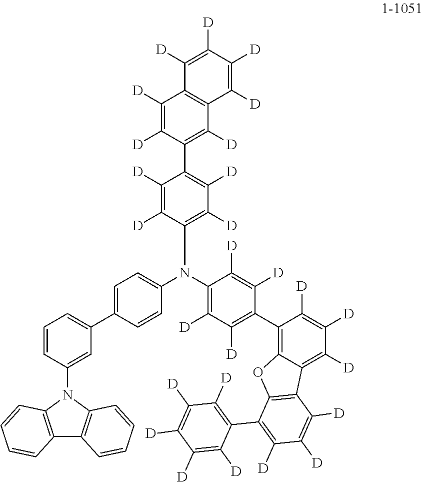 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5c([2H])c([2H])c(-c6c([2H])c([2H])c7c([2H])c([2H])c([2H])c([2H])c7c6[2H])c([2H])c5[2H])c([2H])c4[2H])c([2H])c([2H])c([2H])c23)c([2H])c1[2H]]

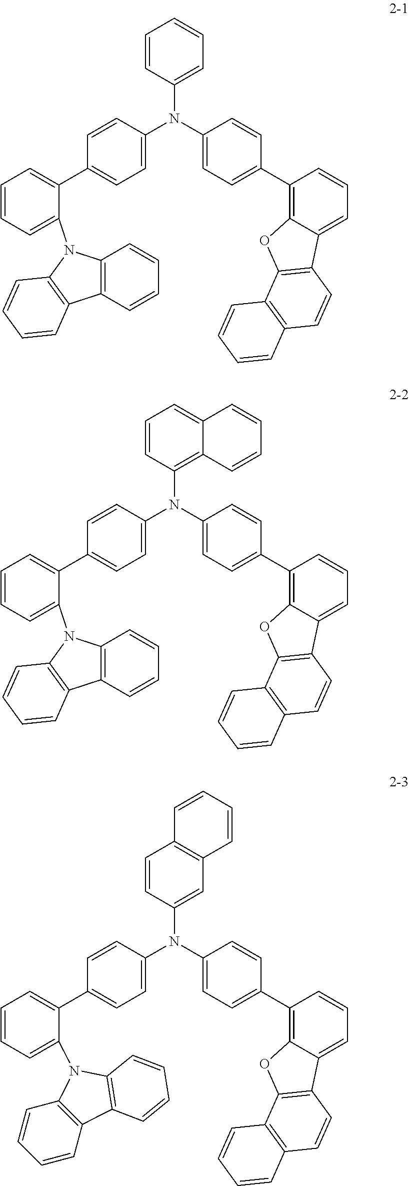

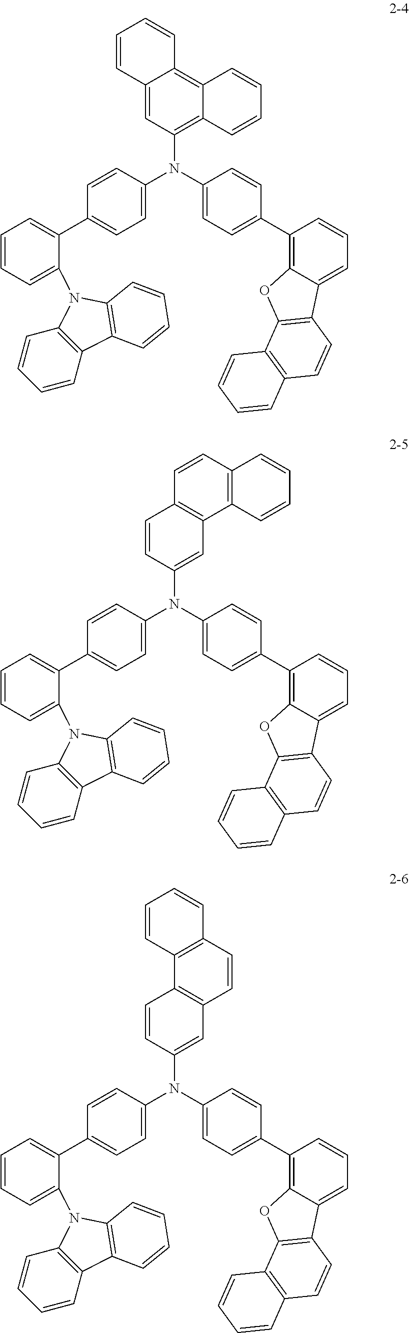

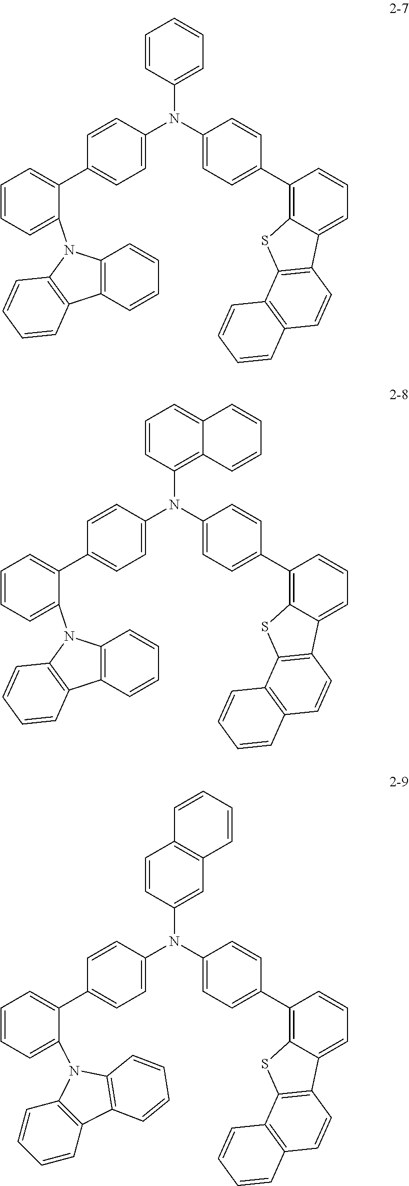

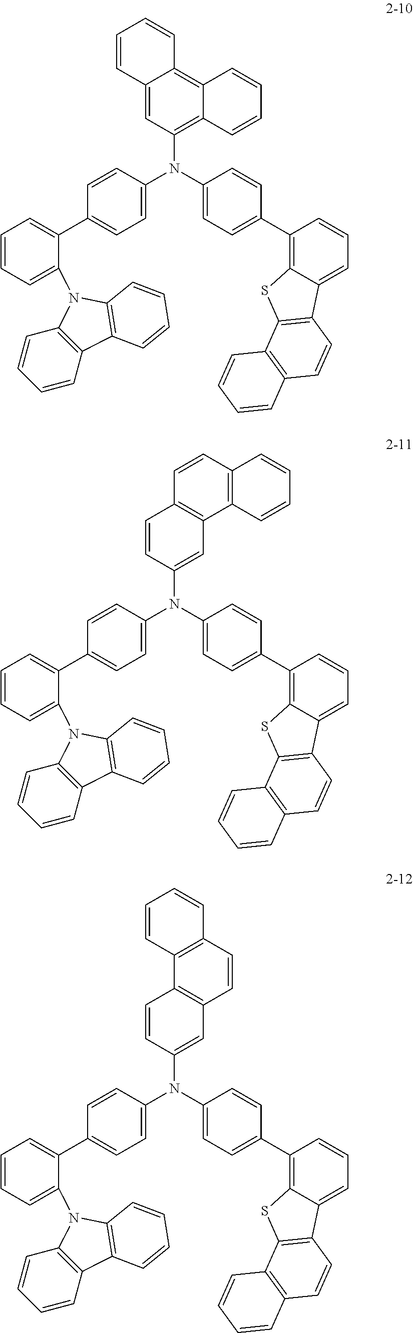

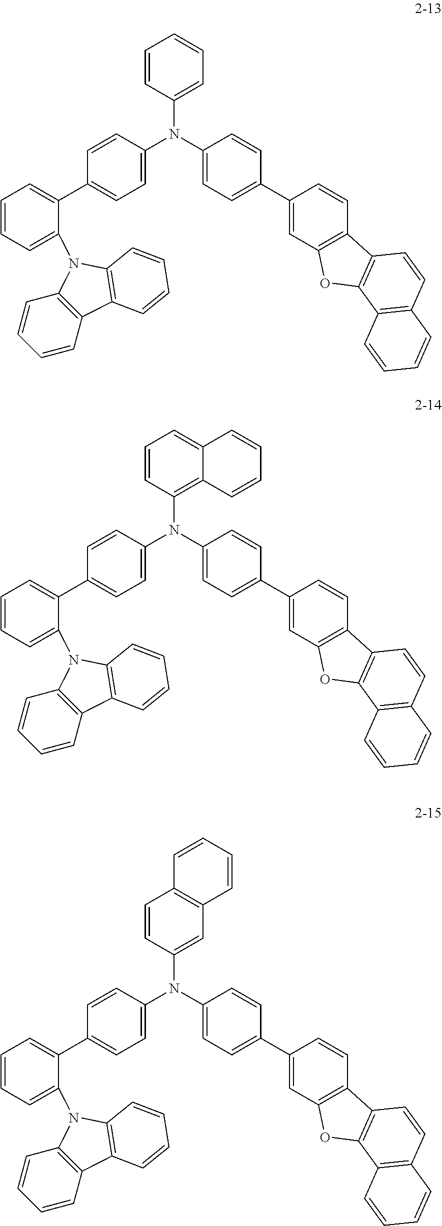 [SMILES: c1ccc(N(c2ccc(-c3ccc4c(c3)oc3c5ccccc5ccc43)cc2)c2ccc(-c3ccccc3-n3c4ccccc4c4ccccc43)cc2)cc1 ; c1ccc(-n2c3ccccc3c3ccccc32)c(-c2ccc(N(c3ccc(-c4ccc5c(c4)oc4c6ccccc6ccc54)cc3)c3cccc4ccccc34)cc2)c1 ; c1ccc(-n2c3ccccc3c3ccccc32)c(-c2ccc(N(c3ccc(-c4ccc5c(c4)oc4c6ccccc6ccc54)cc3)c3ccc4ccccc4c3)cc2)c1]

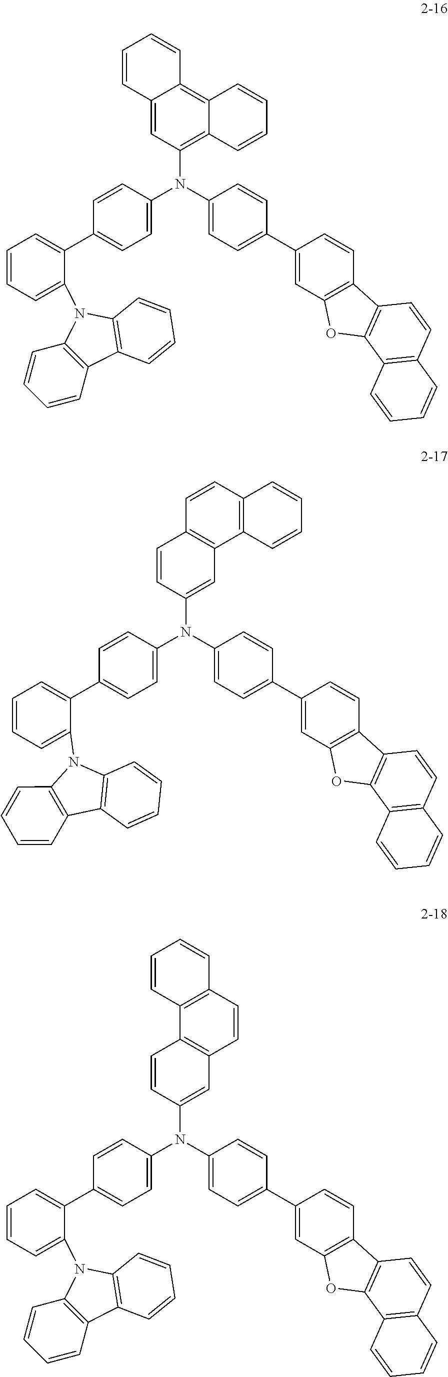 [SMILES: c1ccc(-n2c3ccccc3c3ccccc32)c(-c2ccc(N(c3ccc(-c4ccc5c(c4)oc4c6ccccc6ccc54)cc3)c3cc4ccccc4c4ccccc34)cc2)c1 ; c1ccc(-n2c3ccccc3c3ccccc32)c(-c2ccc(N(c3ccc(-c4ccc5c(c4)oc4c6ccccc6ccc54)cc3)c3ccc4ccc5ccccc5c4c3)cc2)c1 ; c1ccc(-n2c3ccccc3c3ccccc32)c(-c2ccc(N(c3ccc(-c4ccc5c(c4)oc4c6ccccc6ccc54)cc3)c3ccc4c(ccc5ccccc54)c3)cc2)c1]

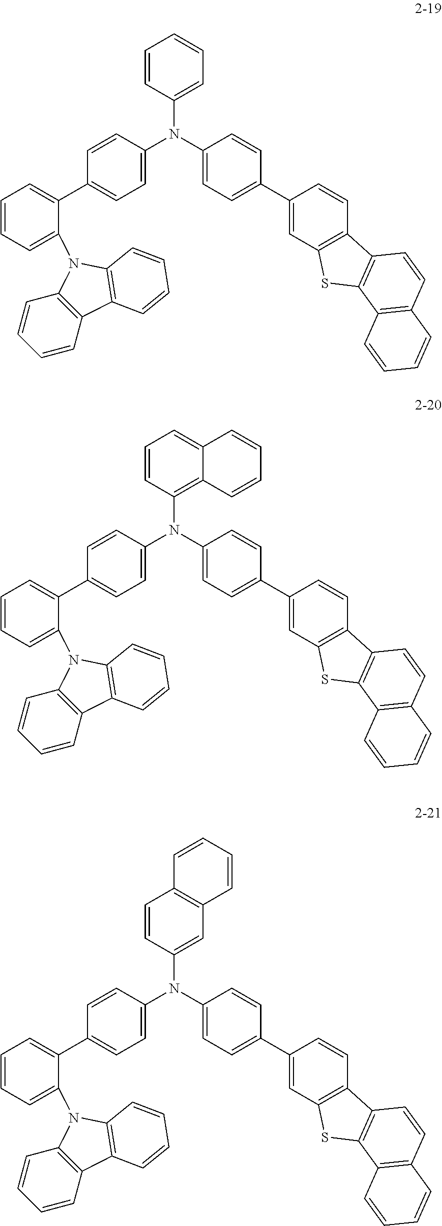 [SMILES: c1ccc(N(c2ccc(-c3ccc4c(c3)sc3c5ccccc5ccc43)cc2)c2ccc(-c3ccccc3-n3c4ccccc4c4ccccc43)cc2)cc1 ; c1ccc(-n2c3ccccc3c3ccccc32)c(-c2ccc(N(c3ccc(-c4ccc5c(c4)sc4c6ccccc6ccc54)cc3)c3cccc4ccccc34)cc2)c1 ; c1ccc(-n2c3ccccc3c3ccccc32)c(-c2ccc(N(c3ccc(-c4ccc5c(c4)sc4c6ccccc6ccc54)cc3)c3ccc4ccccc4c3)cc2)c1]

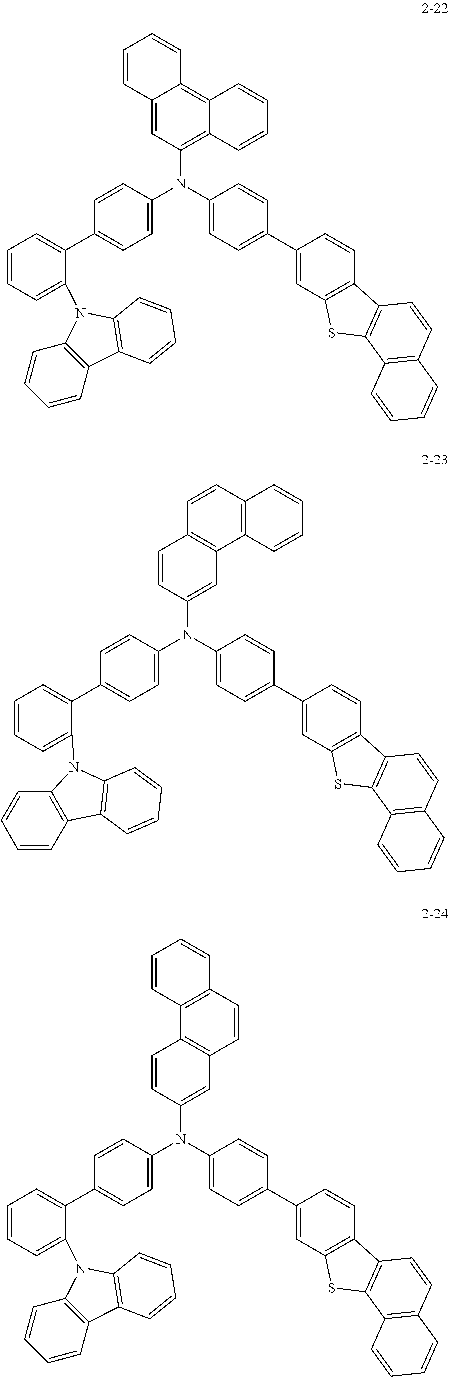 [SMILES: c1ccc(-n2c3ccccc3c3ccccc32)c(-c2ccc(N(c3ccc(-c4ccc5c(c4)sc4c6ccccc6ccc54)cc3)c3cc4ccccc4c4ccccc34)cc2)c1 ; c1ccc(-n2c3ccccc3c3ccccc32)c(-c2ccc(N(c3ccc(-c4ccc5c(c4)sc4c6ccccc6ccc54)cc3)c3ccc4ccc5ccccc5c4c3)cc2)c1 ; c1ccc(-n2c3ccccc3c3ccccc32)c(-c2ccc(N(c3ccc(-c4ccc5c(c4)sc4c6ccccc6ccc54)cc3)c3ccc4c(ccc5ccccc54)c3)cc2)c1]

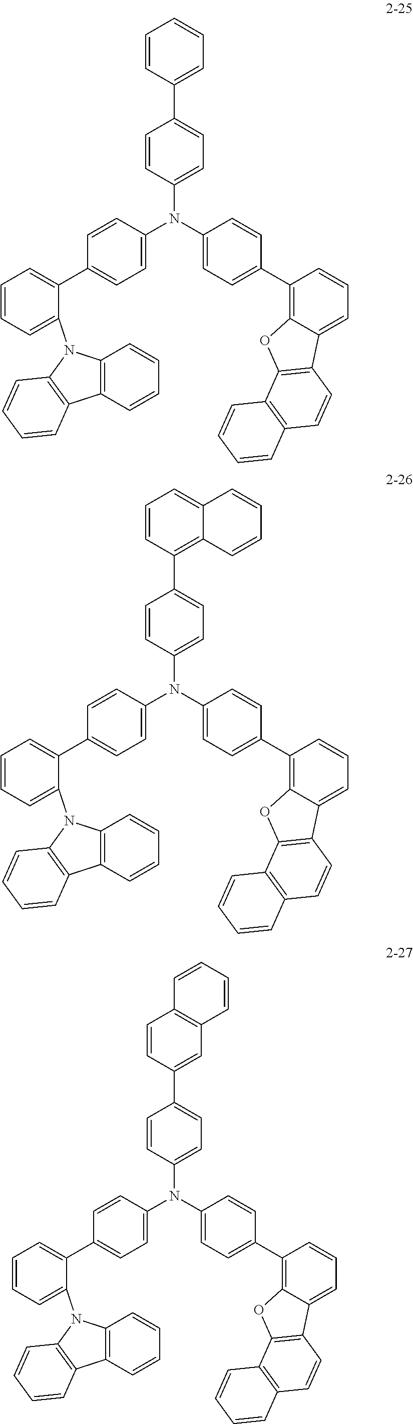

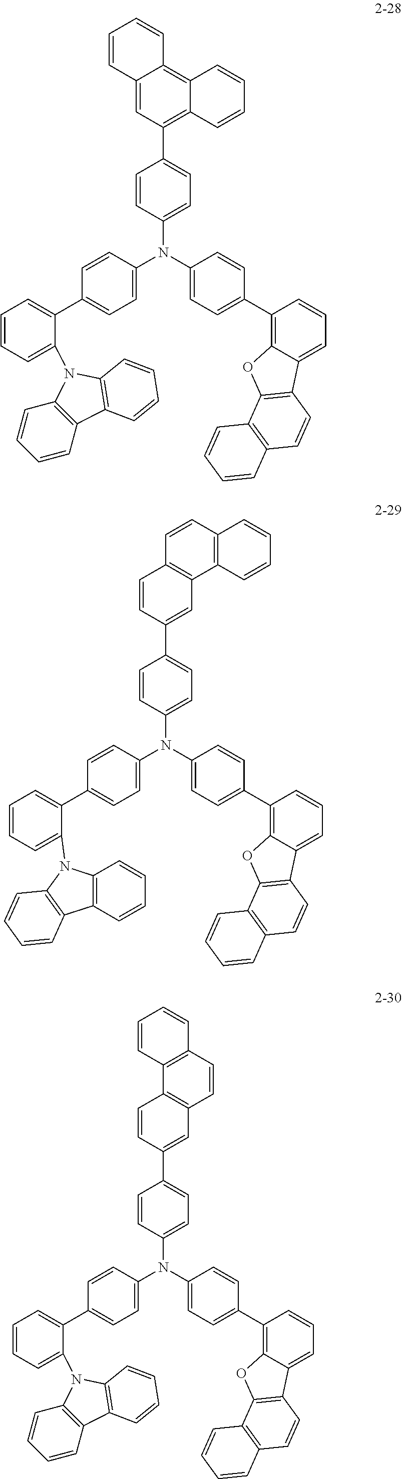 [SMILES: c1ccc(-n2c3ccccc3c3ccccc32)c(-c2ccc(N(c3ccc(-c4cc5ccccc5c5ccccc45)cc3)c3ccc(-c4cccc5c4oc4c6ccccc6ccc54)cc3)cc2)c1 ; c1ccc(-n2c3ccccc3c3ccccc32)c(-c2ccc(N(c3ccc(-c4ccc5ccc6ccccc6c5c4)cc3)c3ccc(-c4cccc5c4oc4c6ccccc6ccc54)cc3)cc2)c1 ; c1ccc(-n2c3ccccc3c3ccccc32)c(-c2ccc(N(c3ccc(-c4ccc5c(ccc6ccccc65)c4)cc3)c3ccc(-c4cccc5c4oc4c6ccccc6ccc54)cc3)cc2)c1]

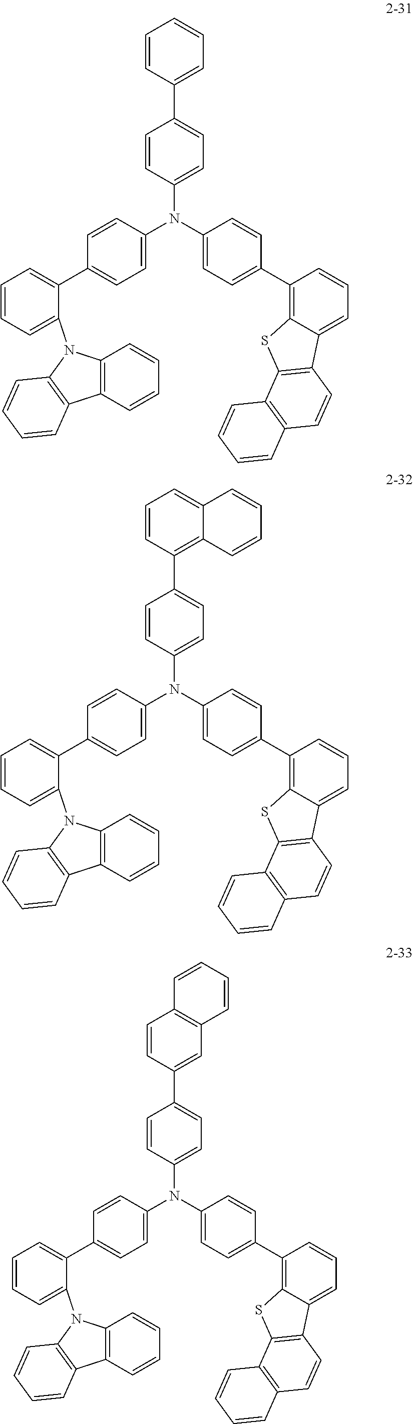

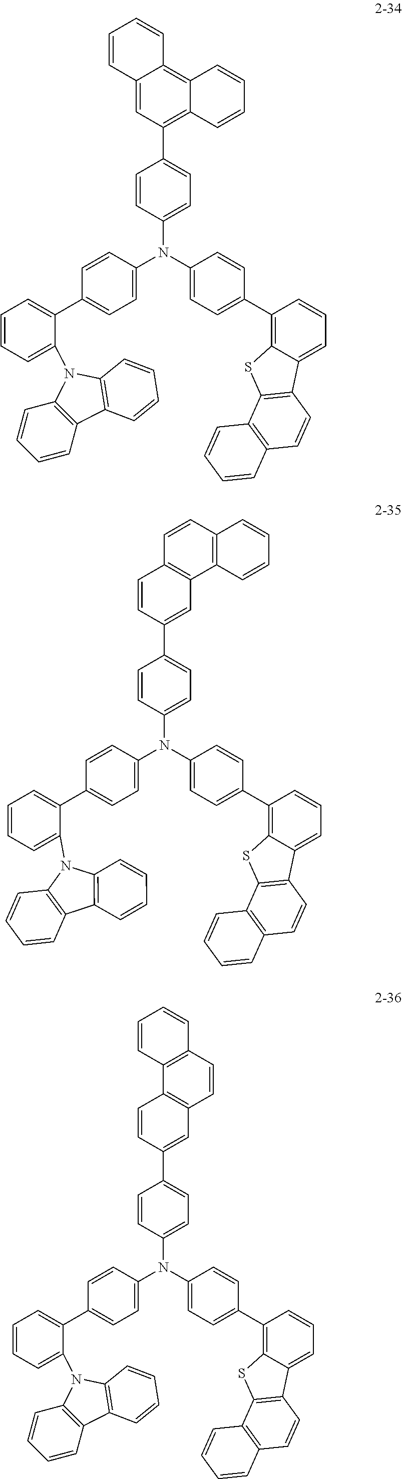 [SMILES: c1ccc(-n2c3ccccc3c3ccccc32)c(-c2ccc(N(c3ccc(-c4cc5ccccc5c5ccccc45)cc3)c3ccc(-c4cccc5c4sc4c6ccccc6ccc54)cc3)cc2)c1 ; c1ccc(-n2c3ccccc3c3ccccc32)c(-c2ccc(N(c3ccc(-c4ccc5ccc6ccccc6c5c4)cc3)c3ccc(-c4cccc5c4sc4c6ccccc6ccc54)cc3)cc2)c1 ; c1ccc(-n2c3ccccc3c3ccccc32)c(-c2ccc(N(c3ccc(-c4ccc5c(ccc6ccccc65)c4)cc3)c3ccc(-c4cccc5c4sc4c6ccccc6ccc54)cc3)cc2)c1]

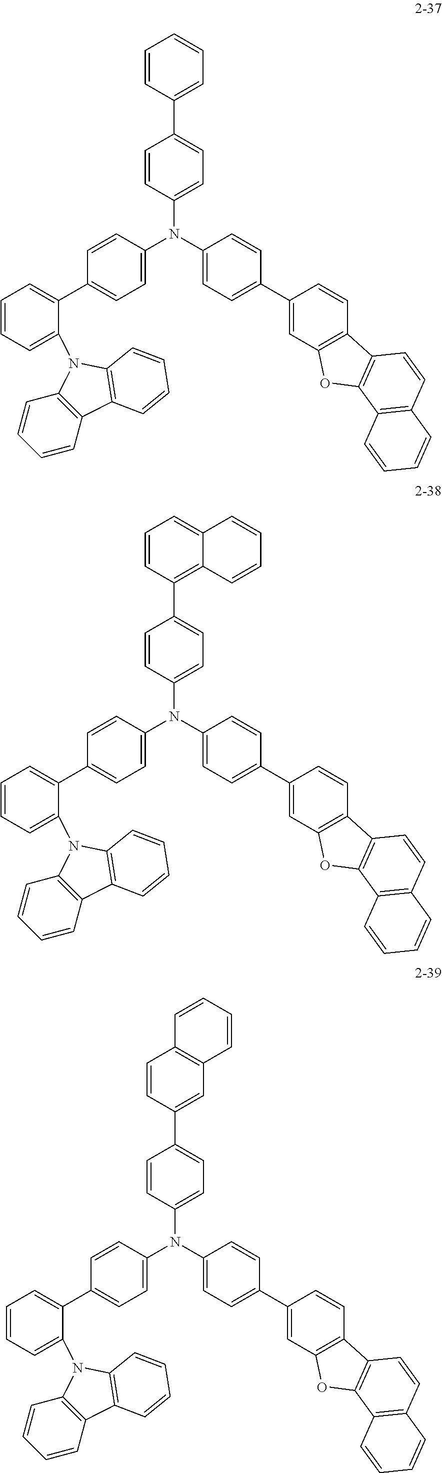 [SMILES: c1ccc(-c2ccc(N(c3ccc(-c4ccc5c(c4)oc4c6ccccc6ccc54)cc3)c3ccc(-c4ccccc4-n4c5ccccc5c5ccccc54)cc3)cc2)cc1 ; c1ccc(-n2c3ccccc3c3ccccc32)c(-c2ccc(N(c3ccc(-c4ccc5c(c4)oc4c6ccccc6ccc54)cc3)c3ccc(-c4cccc5ccccc45)cc3)cc2)c1 ; c1ccc(-n2c3ccccc3c3ccccc32)c(-c2ccc(N(c3ccc(-c4ccc5ccccc5c4)cc3)c3ccc(-c4ccc5c(c4)oc4c6ccccc6ccc54)cc3)cc2)c1]

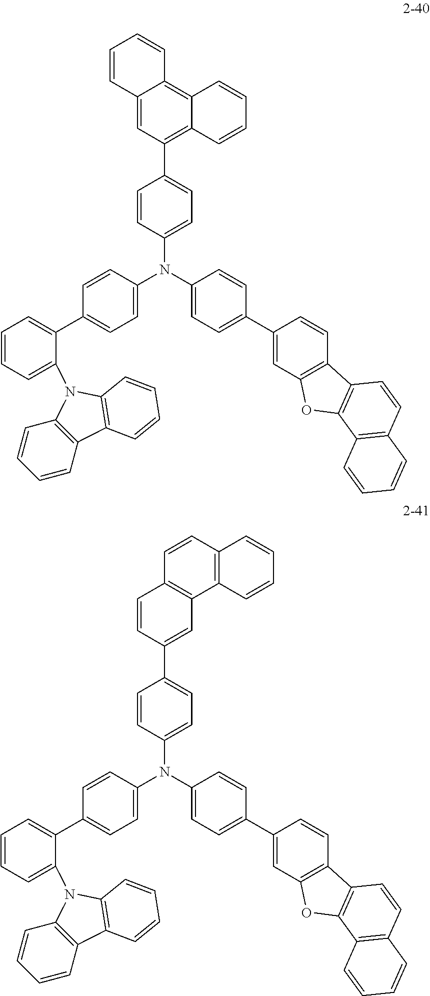 [SMILES: c1ccc(-n2c3ccccc3c3ccccc32)c(-c2ccc(N(c3ccc(-c4ccc5c(c4)oc4c6ccccc6ccc54)cc3)c3ccc(-c4cc5ccccc5c5ccccc45)cc3)cc2)c1 ; c1ccc(-n2c3ccccc3c3ccccc32)c(-c2ccc(N(c3ccc(-c4ccc5c(c4)oc4c6ccccc6ccc54)cc3)c3ccc(-c4ccc5ccc6ccccc6c5c4)cc3)cc2)c1]

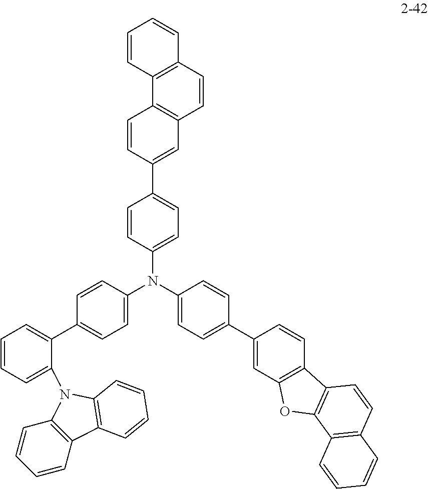 [SMILES: c1ccc(-n2c3ccccc3c3ccccc32)c(-c2ccc(N(c3ccc(-c4ccc5c(ccc6ccccc65)c4)cc3)c3ccc(-c4ccc5c(c4)oc4c6ccccc6ccc54)cc3)cc2)c1]

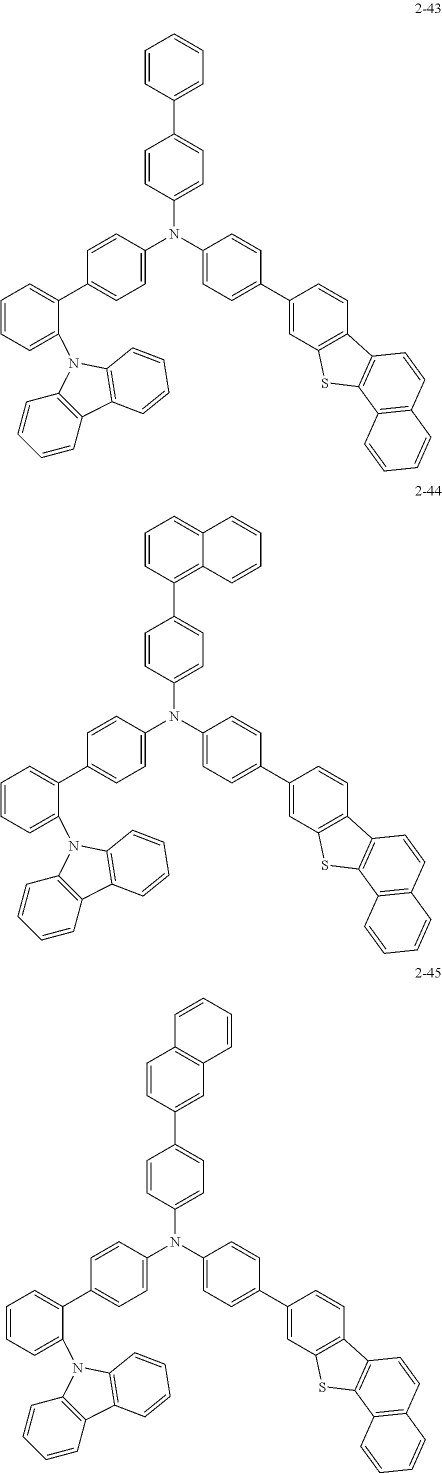 [SMILES: c1ccc(-c2ccc(N(c3ccc(-c4ccc5c(c4)sc4c6ccccc6ccc54)cc3)c3ccc(-c4ccccc4-n4c5ccccc5c5ccccc54)cc3)cc2)cc1 ; c1ccc(-n2c3ccccc3c3ccccc32)c(-c2ccc(N(c3ccc(-c4ccc5c(c4)sc4c6ccccc6ccc54)cc3)c3ccc(-c4cccc5ccccc45)cc3)cc2)c1 ; c1ccc(-n2c3ccccc3c3ccccc32)c(-c2ccc(N(c3ccc(-c4ccc5ccccc5c4)cc3)c3ccc(-c4ccc5c(c4)sc4c6ccccc6ccc54)cc3)cc2)c1]

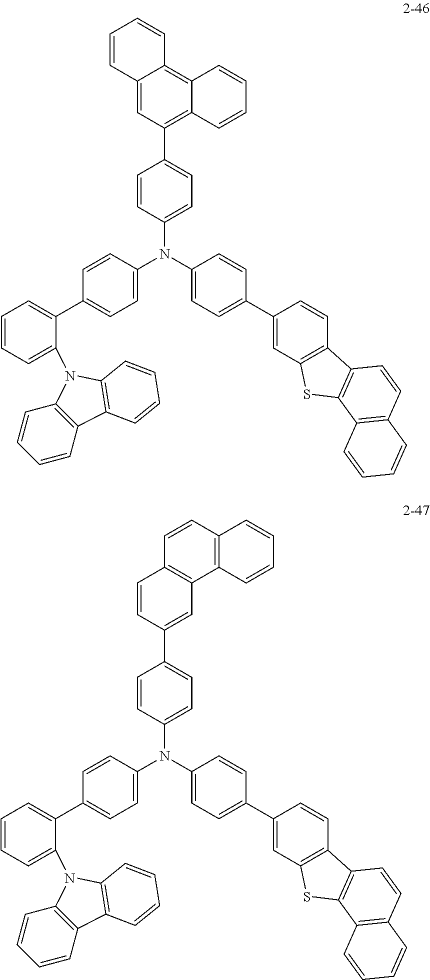 [SMILES: c1ccc(-n2c3ccccc3c3ccccc32)c(-c2ccc(N(c3ccc(-c4ccc5c(c4)sc4c6ccccc6ccc54)cc3)c3ccc(-c4cc5ccccc5c5ccccc45)cc3)cc2)c1 ; c1ccc(-n2c3ccccc3c3ccccc32)c(-c2ccc(N(c3ccc(-c4ccc5c(c4)sc4c6ccccc6ccc54)cc3)c3ccc(-c4ccc5ccc6ccccc6c5c4)cc3)cc2)c1]

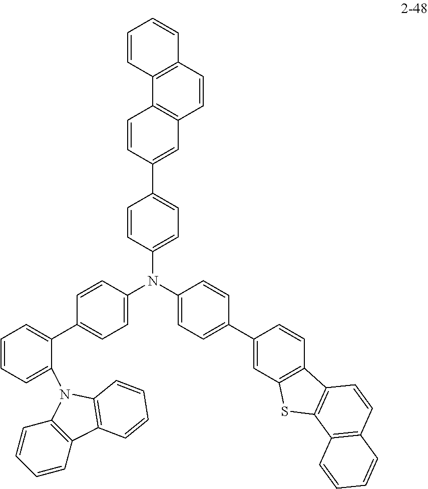 [SMILES: c1ccc(-n2c3ccccc3c3ccccc32)c(-c2ccc(N(c3ccc(-c4ccc5c(ccc6ccccc65)c4)cc3)c3ccc(-c4ccc5c(c4)sc4c6ccccc6ccc54)cc3)cc2)c1]

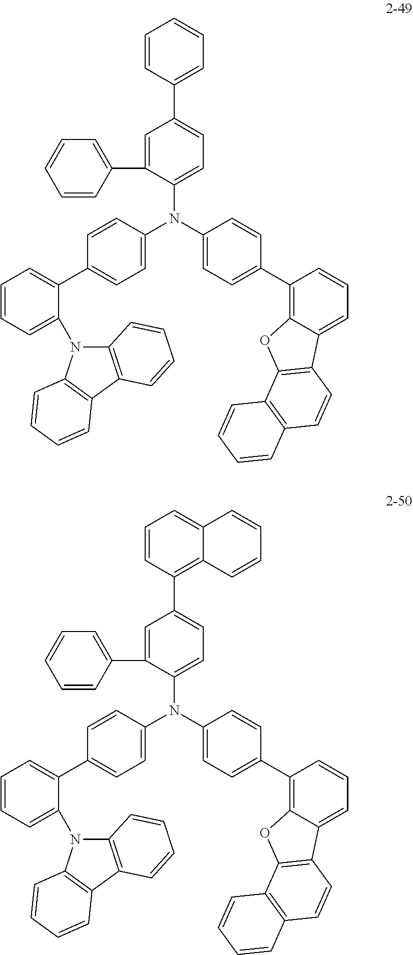 [SMILES: c1ccc(-c2ccc(N(c3ccc(-c4ccccc4-n4c5ccccc5c5ccccc54)cc3)c3ccc(-c4cccc5c4oc4c6ccccc6ccc54)cc3)c(-c3ccccc3)c2)cc1 ; c1ccc(-c2cc(-c3cccc4ccccc34)ccc2N(c2ccc(-c3ccccc3-n3c4ccccc4c4ccccc43)cc2)c2ccc(-c3cccc4c3oc3c5ccccc5ccc43)cc2)cc1]

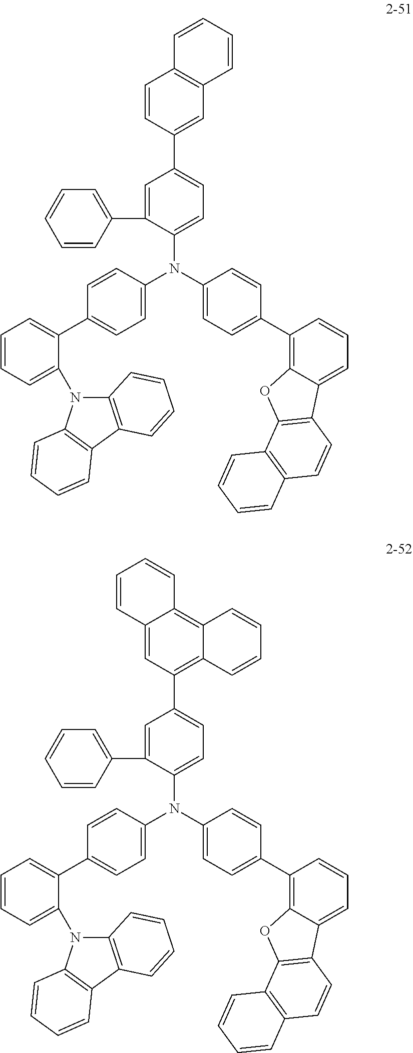 [SMILES: c1ccc(-c2cc(-c3ccc4ccccc4c3)ccc2N(c2ccc(-c3ccccc3-n3c4ccccc4c4ccccc43)cc2)c2ccc(-c3cccc4c3oc3c5ccccc5ccc43)cc2)cc1 ; c1ccc(-c2cc(-c3cc4ccccc4c4ccccc34)ccc2N(c2ccc(-c3ccccc3-n3c4ccccc4c4ccccc43)cc2)c2ccc(-c3cccc4c3oc3c5ccccc5ccc43)cc2)cc1]

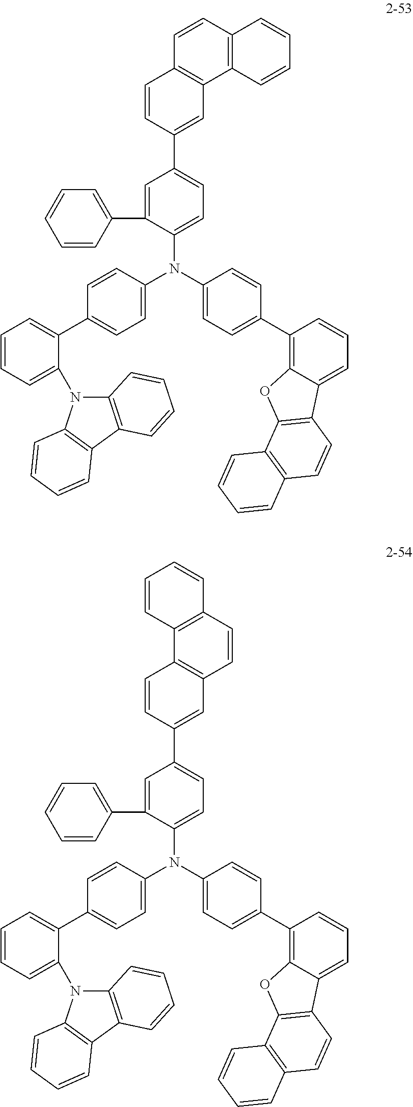 [SMILES: c1ccc(-c2cc(-c3ccc4ccc5ccccc5c4c3)ccc2N(c2ccc(-c3ccccc3-n3c4ccccc4c4ccccc43)cc2)c2ccc(-c3cccc4c3oc3c5ccccc5ccc43)cc2)cc1 ; c1ccc(-c2cc(-c3ccc4c(ccc5ccccc54)c3)ccc2N(c2ccc(-c3ccccc3-n3c4ccccc4c4ccccc43)cc2)c2ccc(-c3cccc4c3oc3c5ccccc5ccc43)cc2)cc1]

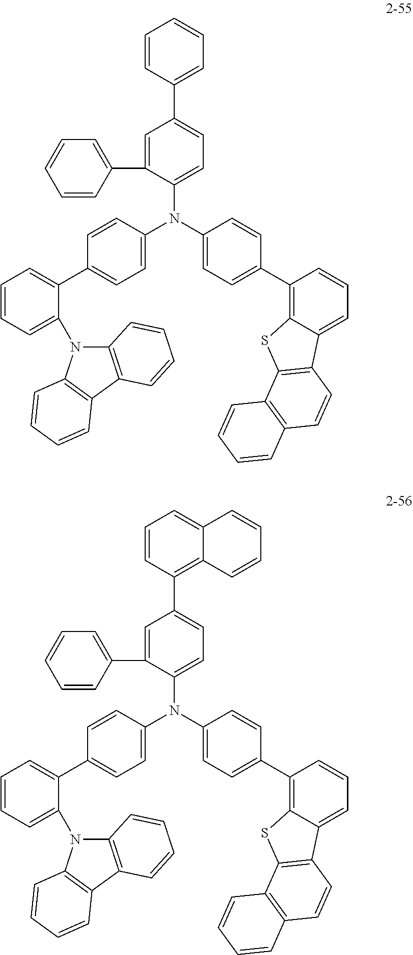 [SMILES: c1ccc(-c2ccc(N(c3ccc(-c4ccccc4-n4c5ccccc5c5ccccc54)cc3)c3ccc(-c4cccc5c4sc4c6ccccc6ccc54)cc3)c(-c3ccccc3)c2)cc1 ; c1ccc(-c2cc(-c3cccc4ccccc34)ccc2N(c2ccc(-c3ccccc3-n3c4ccccc4c4ccccc43)cc2)c2ccc(-c3cccc4c3sc3c5ccccc5ccc43)cc2)cc1]

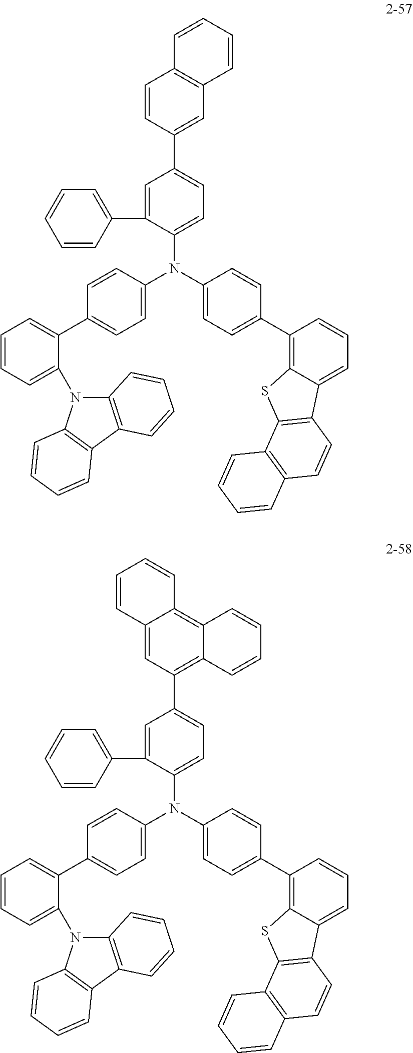 [SMILES: c1ccc(-c2cc(-c3ccc4ccccc4c3)ccc2N(c2ccc(-c3ccccc3-n3c4ccccc4c4ccccc43)cc2)c2ccc(-c3cccc4c3sc3c5ccccc5ccc43)cc2)cc1 ; c1ccc(-c2cc(-c3cc4ccccc4c4ccccc34)ccc2N(c2ccc(-c3ccccc3-n3c4ccccc4c4ccccc43)cc2)c2ccc(-c3cccc4c3sc3c5ccccc5ccc43)cc2)cc1]

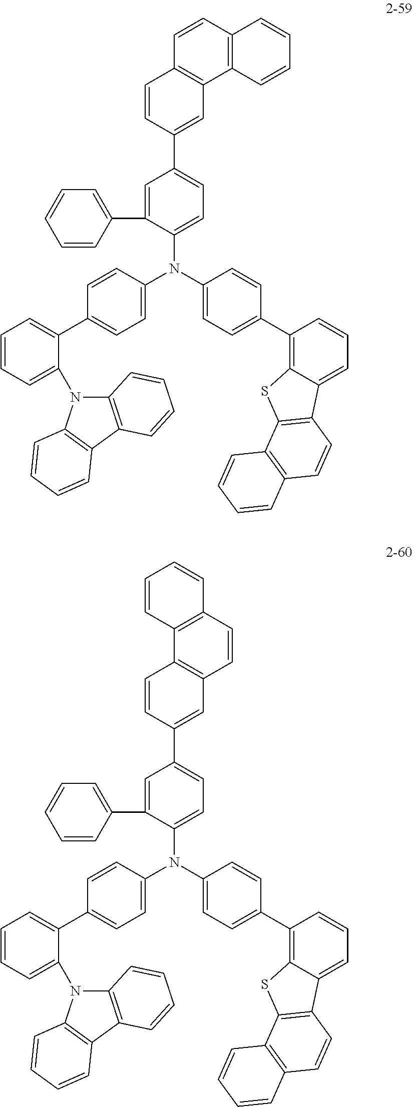 [SMILES: c1ccc(-c2cc(-c3ccc4ccc5ccccc5c4c3)ccc2N(c2ccc(-c3ccccc3-n3c4ccccc4c4ccccc43)cc2)c2ccc(-c3cccc4c3sc3c5ccccc5ccc43)cc2)cc1 ; c1ccc(-c2cc(-c3ccc4c(ccc5ccccc54)c3)ccc2N(c2ccc(-c3ccccc3-n3c4ccccc4c4ccccc43)cc2)c2ccc(-c3cccc4c3sc3c5ccccc5ccc43)cc2)cc1]

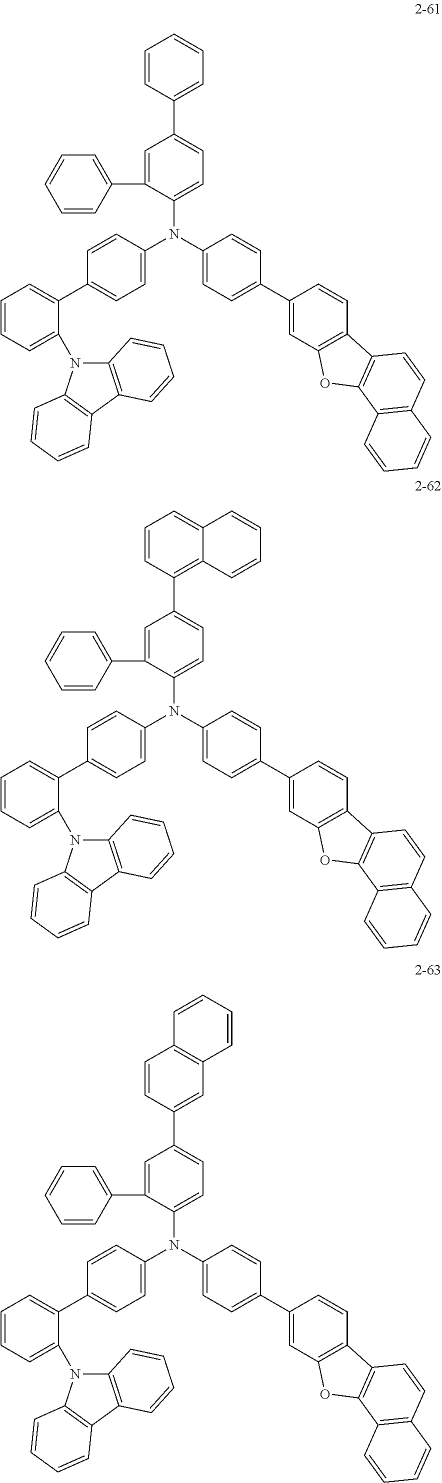 [SMILES: c1ccc(-c2ccc(N(c3ccc(-c4ccc5c(c4)oc4c6ccccc6ccc54)cc3)c3ccc(-c4ccccc4-n4c5ccccc5c5ccccc54)cc3)c(-c3ccccc3)c2)cc1 ; c1ccc(-c2cc(-c3cccc4ccccc34)ccc2N(c2ccc(-c3ccc4c(c3)oc3c5ccccc5ccc43)cc2)c2ccc(-c3ccccc3-n3c4ccccc4c4ccccc43)cc2)cc1 ; c1ccc(-c2cc(-c3ccc4ccccc4c3)ccc2N(c2ccc(-c3ccc4c(c3)oc3c5ccccc5ccc43)cc2)c2ccc(-c3ccccc3-n3c4ccccc4c4ccccc43)cc2)cc1]

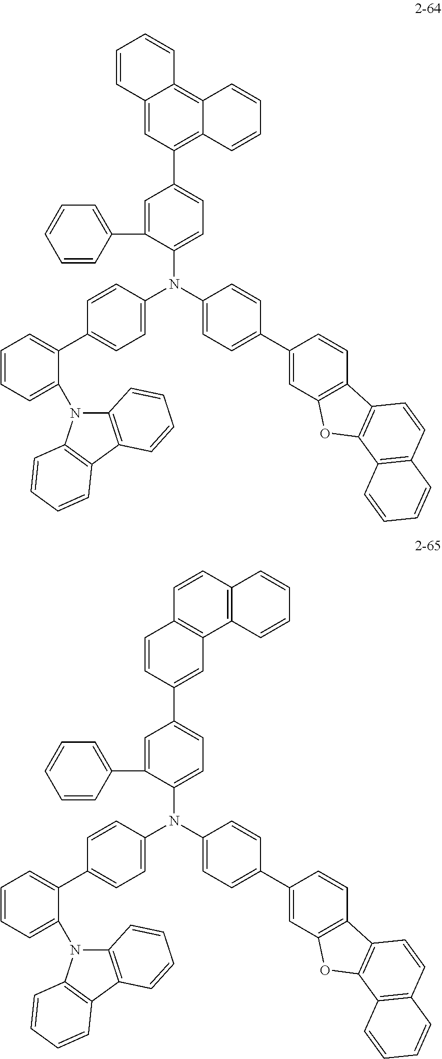 [SMILES: c1ccc(-c2cc(-c3cc4ccccc4c4ccccc34)ccc2N(c2ccc(-c3ccc4c(c3)oc3c5ccccc5ccc43)cc2)c2ccc(-c3ccccc3-n3c4ccccc4c4ccccc43)cc2)cc1 ; c1ccc(-c2cc(-c3ccc4ccc5ccccc5c4c3)ccc2N(c2ccc(-c3ccc4c(c3)oc3c5ccccc5ccc43)cc2)c2ccc(-c3ccccc3-n3c4ccccc4c4ccccc43)cc2)cc1]

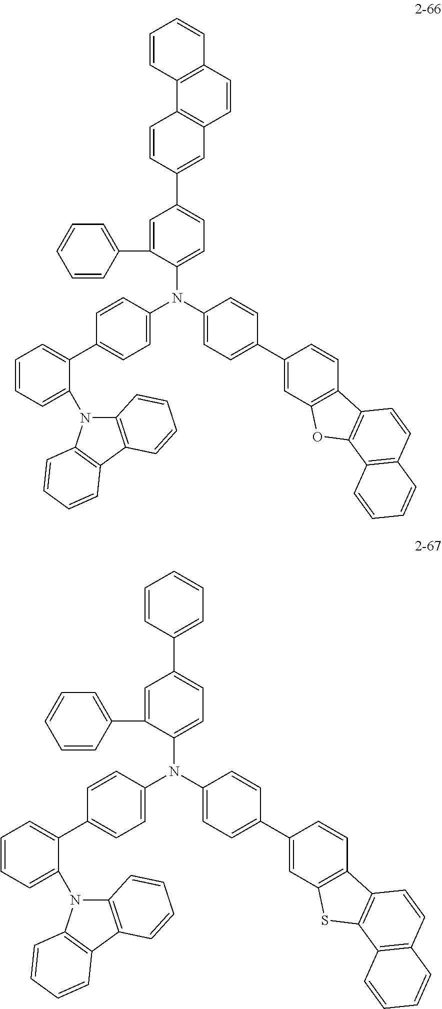 [SMILES: c1ccc(-c2cc(-c3ccc4c(ccc5ccccc54)c3)ccc2N(c2ccc(-c3ccc4c(c3)oc3c5ccccc5ccc43)cc2)c2ccc(-c3ccccc3-n3c4ccccc4c4ccccc43)cc2)cc1 ; c1ccc(-c2ccc(N(c3ccc(-c4ccc5c(c4)sc4c6ccccc6ccc54)cc3)c3ccc(-c4ccccc4-n4c5ccccc5c5ccccc54)cc3)c(-c3ccccc3)c2)cc1]

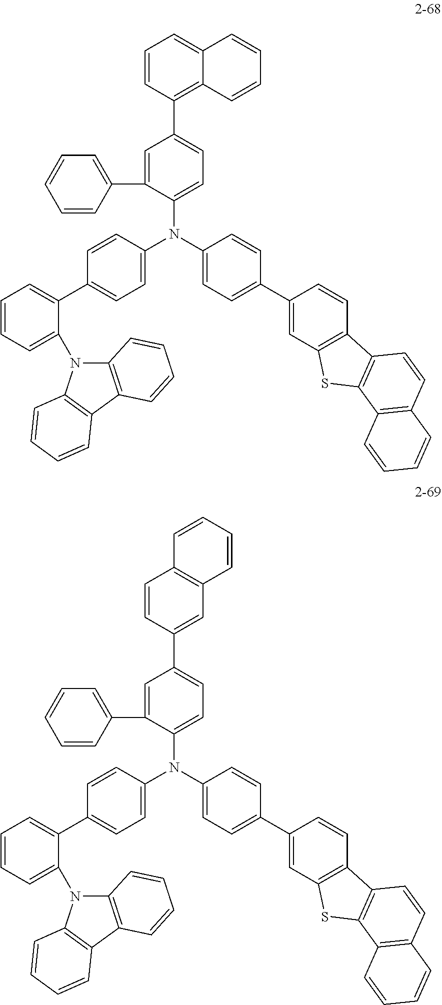 [SMILES: c1ccc(-c2cc(-c3cccc4ccccc34)ccc2N(c2ccc(-c3ccc4c(c3)sc3c5ccccc5ccc43)cc2)c2ccc(-c3ccccc3-n3c4ccccc4c4ccccc43)cc2)cc1 ; c1ccc(-c2cc(-c3ccc4ccccc4c3)ccc2N(c2ccc(-c3ccc4c(c3)sc3c5ccccc5ccc43)cc2)c2ccc(-c3ccccc3-n3c4ccccc4c4ccccc43)cc2)cc1]

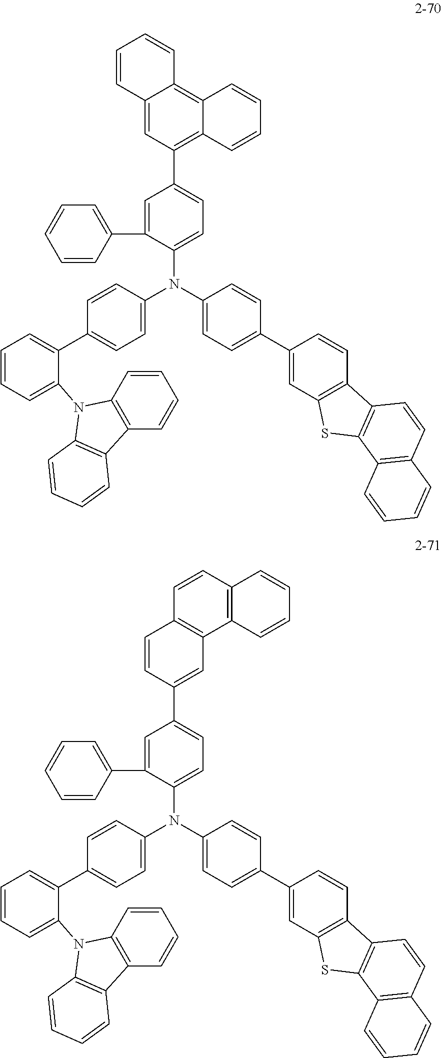 [SMILES: c1ccc(-c2cc(-c3cc4ccccc4c4ccccc34)ccc2N(c2ccc(-c3ccc4c(c3)sc3c5ccccc5ccc43)cc2)c2ccc(-c3ccccc3-n3c4ccccc4c4ccccc43)cc2)cc1 ; c1ccc(-c2cc(-c3ccc4ccc5ccccc5c4c3)ccc2N(c2ccc(-c3ccc4c(c3)sc3c5ccccc5ccc43)cc2)c2ccc(-c3ccccc3-n3c4ccccc4c4ccccc43)cc2)cc1]

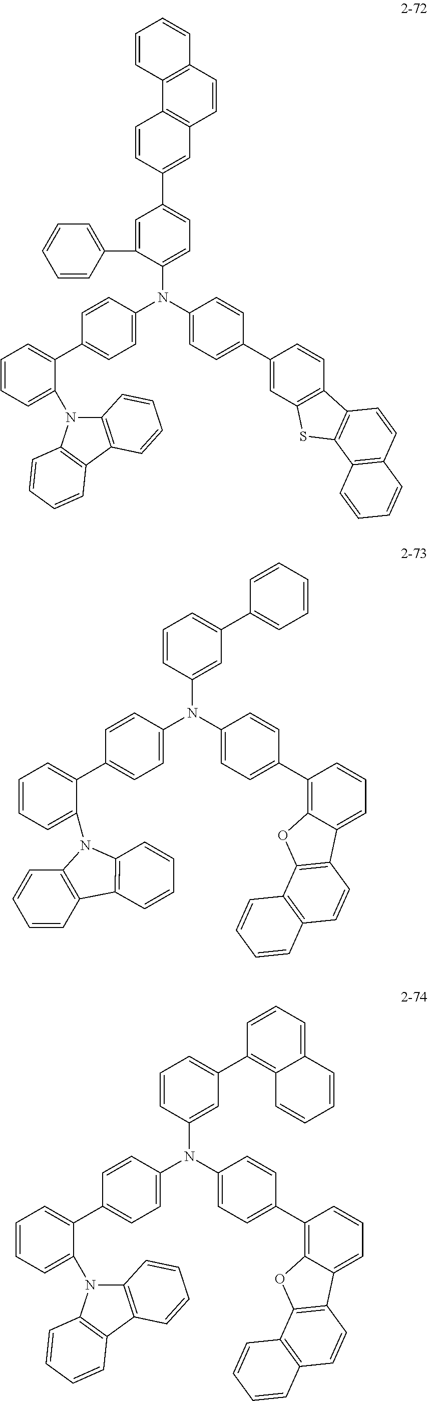 [SMILES: c1ccc(-c2cc(-c3ccc4c(ccc5ccccc54)c3)ccc2N(c2ccc(-c3ccc4c(c3)sc3c5ccccc5ccc43)cc2)c2ccc(-c3ccccc3-n3c4ccccc4c4ccccc43)cc2)cc1 ; c1ccc(-c2cccc(N(c3ccc(-c4ccccc4-n4c5ccccc5c5ccccc54)cc3)c3ccc(-c4cccc5c4oc4c6ccccc6ccc54)cc3)c2)cc1 ; c1cc(-c2cccc3ccccc23)cc(N(c2ccc(-c3ccccc3-n3c4ccccc4c4ccccc43)cc2)c2ccc(-c3cccc4c3oc3c5ccccc5ccc43)cc2)c1]

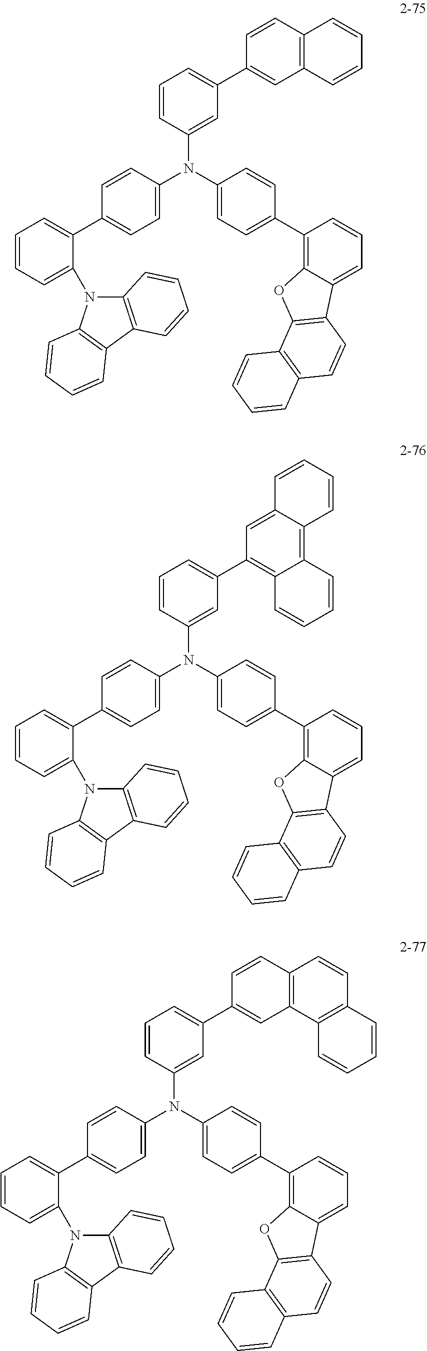 [SMILES: c1cc(-c2ccc3ccccc3c2)cc(N(c2ccc(-c3ccccc3-n3c4ccccc4c4ccccc43)cc2)c2ccc(-c3cccc4c3oc3c5ccccc5ccc43)cc2)c1 ; c1cc(-c2cc3ccccc3c3ccccc23)cc(N(c2ccc(-c3ccccc3-n3c4ccccc4c4ccccc43)cc2)c2ccc(-c3cccc4c3oc3c5ccccc5ccc43)cc2)c1 ; c1cc(-c2ccc3ccc4ccccc4c3c2)cc(N(c2ccc(-c3ccccc3-n3c4ccccc4c4ccccc43)cc2)c2ccc(-c3cccc4c3oc3c5ccccc5ccc43)cc2)c1]

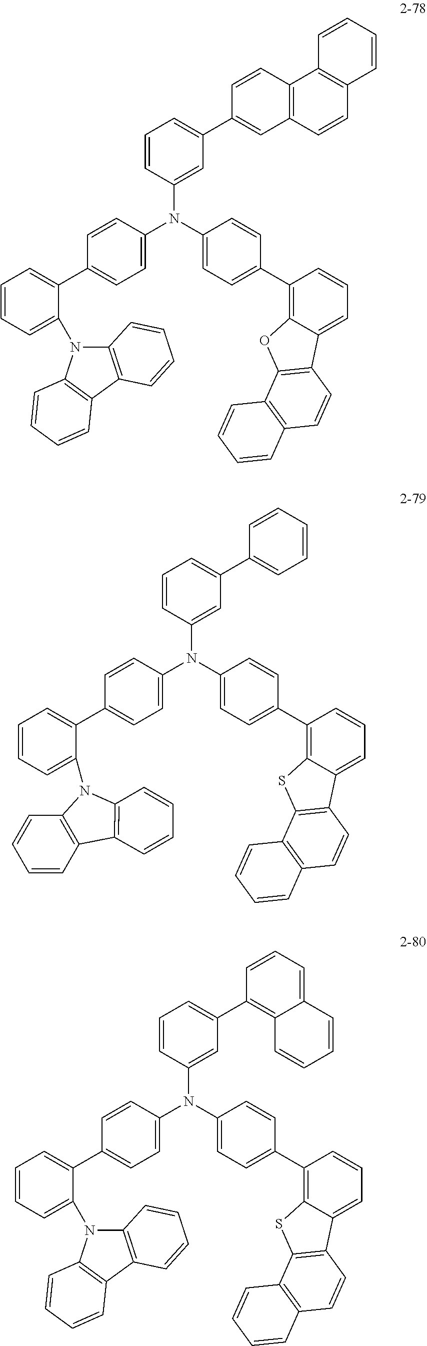 [SMILES: c1cc(-c2ccc3c(ccc4ccccc43)c2)cc(N(c2ccc(-c3ccccc3-n3c4ccccc4c4ccccc43)cc2)c2ccc(-c3cccc4c3oc3c5ccccc5ccc43)cc2)c1 ; c1ccc(-c2cccc(N(c3ccc(-c4ccccc4-n4c5ccccc5c5ccccc54)cc3)c3ccc(-c4cccc5c4sc4c6ccccc6ccc54)cc3)c2)cc1 ; c1cc(-c2cccc3ccccc23)cc(N(c2ccc(-c3ccccc3-n3c4ccccc4c4ccccc43)cc2)c2ccc(-c3cccc4c3sc3c5ccccc5ccc43)cc2)c1]

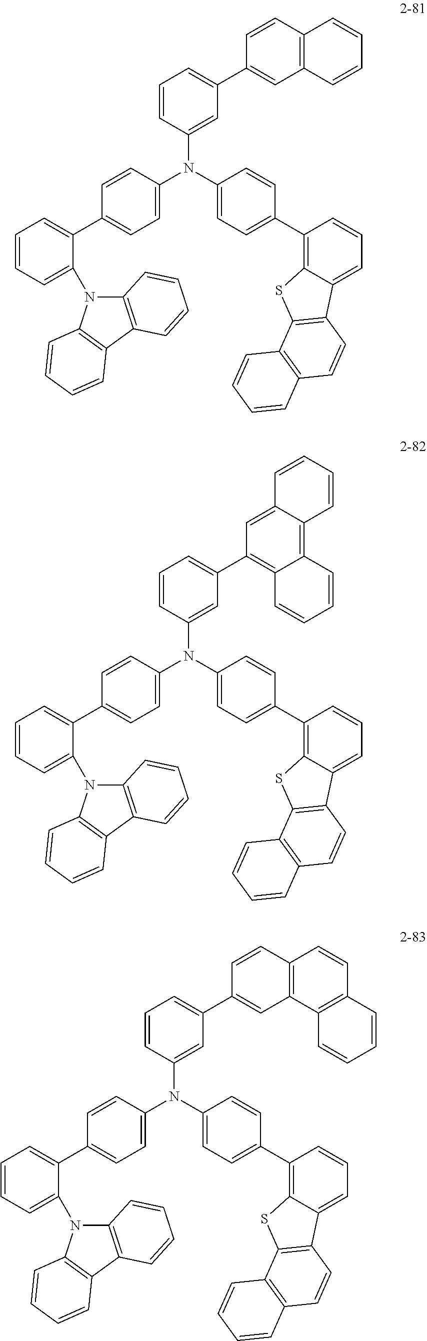 [SMILES: c1cc(-c2ccc3ccccc3c2)cc(N(c2ccc(-c3ccccc3-n3c4ccccc4c4ccccc43)cc2)c2ccc(-c3cccc4c3sc3c5ccccc5ccc43)cc2)c1 ; c1cc(-c2cc3ccccc3c3ccccc23)cc(N(c2ccc(-c3ccccc3-n3c4ccccc4c4ccccc43)cc2)c2ccc(-c3cccc4c3sc3c5ccccc5ccc43)cc2)c1 ; c1cc(-c2ccc3ccc4ccccc4c3c2)cc(N(c2ccc(-c3ccccc3-n3c4ccccc4c4ccccc43)cc2)c2ccc(-c3cccc4c3sc3c5ccccc5ccc43)cc2)c1]

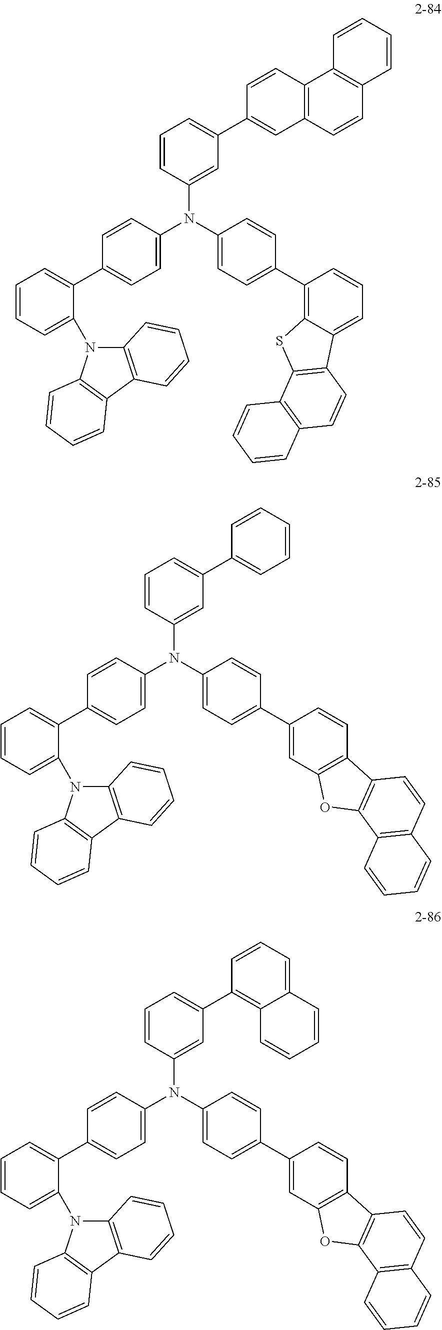

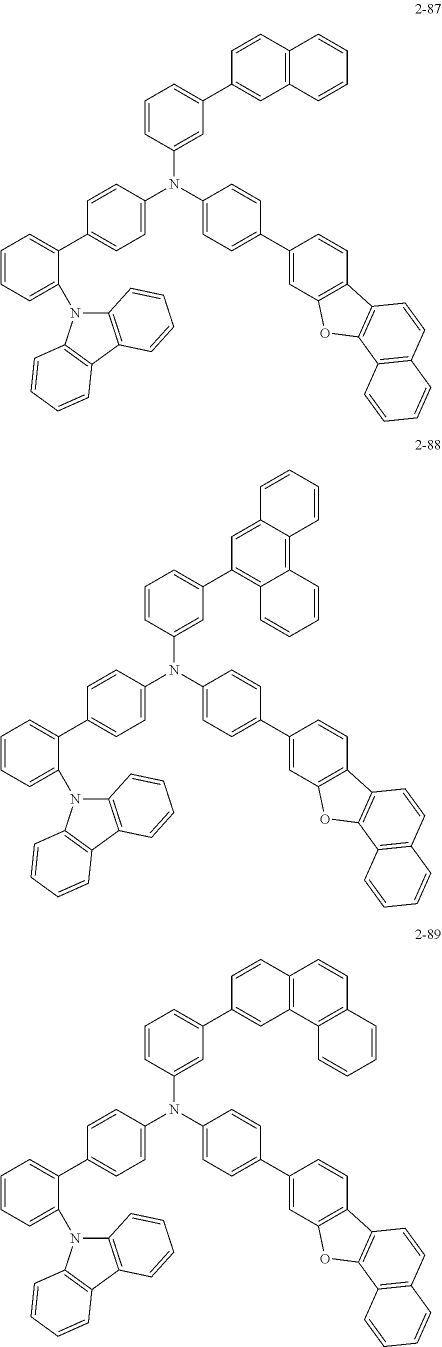 [SMILES: c1cc(-c2ccc3ccccc3c2)cc(N(c2ccc(-c3ccc4c(c3)oc3c5ccccc5ccc43)cc2)c2ccc(-c3ccccc3-n3c4ccccc4c4ccccc43)cc2)c1 ; c1cc(-c2cc3ccccc3c3ccccc23)cc(N(c2ccc(-c3ccc4c(c3)oc3c5ccccc5ccc43)cc2)c2ccc(-c3ccccc3-n3c4ccccc4c4ccccc43)cc2)c1 ; c1cc(-c2ccc3ccc4ccccc4c3c2)cc(N(c2ccc(-c3ccc4c(c3)oc3c5ccccc5ccc43)cc2)c2ccc(-c3ccccc3-n3c4ccccc4c4ccccc43)cc2)c1]

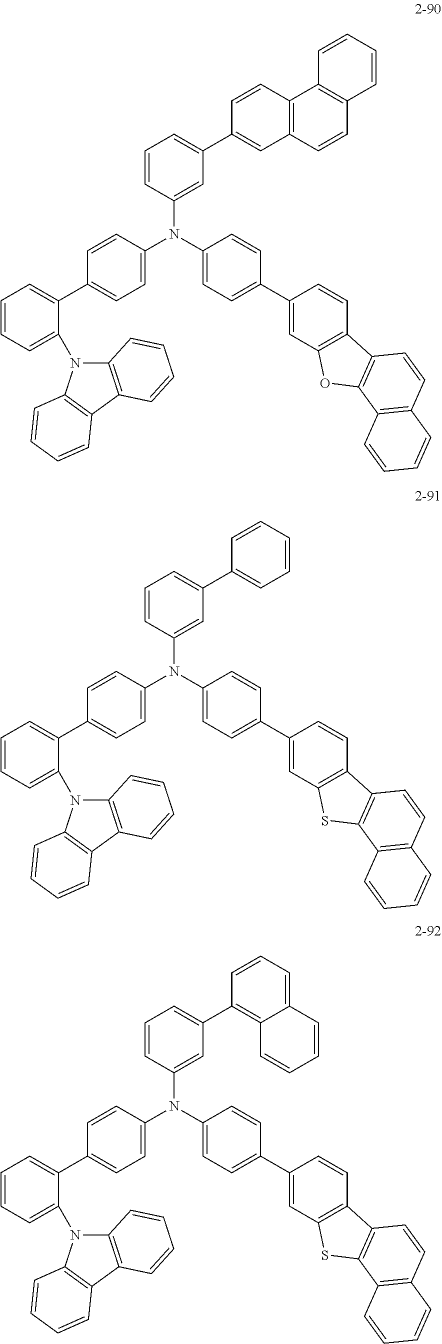 [SMILES: c1cc(-c2ccc3c(ccc4ccccc43)c2)cc(N(c2ccc(-c3ccc4c(c3)oc3c5ccccc5ccc43)cc2)c2ccc(-c3ccccc3-n3c4ccccc4c4ccccc43)cc2)c1 ; c1ccc(-c2cccc(N(c3ccc(-c4ccc5c(c4)sc4c6ccccc6ccc54)cc3)c3ccc(-c4ccccc4-n4c5ccccc5c5ccccc54)cc3)c2)cc1 ; c1cc(-c2cccc3ccccc23)cc(N(c2ccc(-c3ccc4c(c3)sc3c5ccccc5ccc43)cc2)c2ccc(-c3ccccc3-n3c4ccccc4c4ccccc43)cc2)c1]

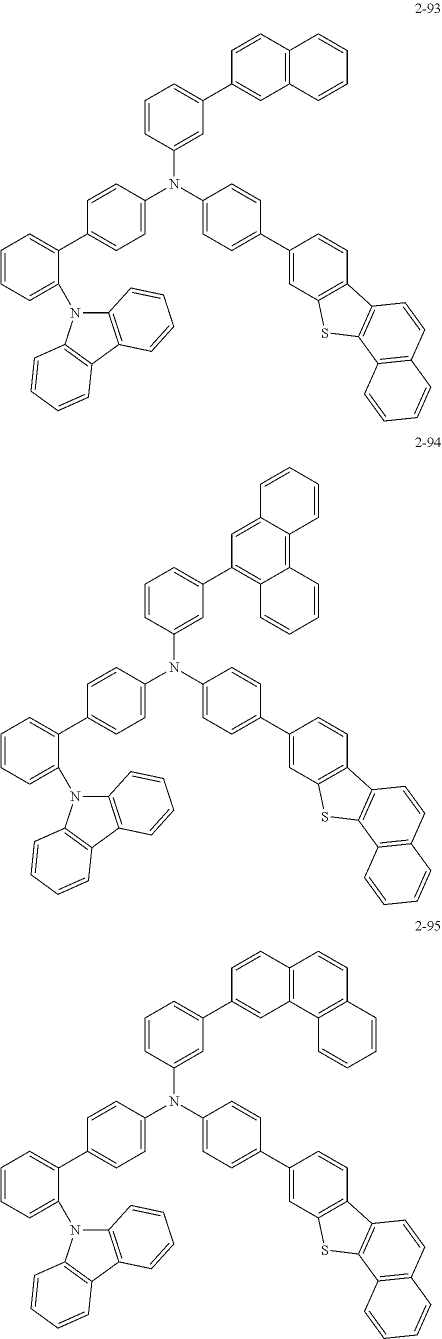 [SMILES: c1cc(-c2ccc3ccccc3c2)cc(N(c2ccc(-c3ccc4c(c3)sc3c5ccccc5ccc43)cc2)c2ccc(-c3ccccc3-n3c4ccccc4c4ccccc43)cc2)c1 ; c1cc(-c2cc3ccccc3c3ccccc23)cc(N(c2ccc(-c3ccc4c(c3)sc3c5ccccc5ccc43)cc2)c2ccc(-c3ccccc3-n3c4ccccc4c4ccccc43)cc2)c1 ; c1cc(-c2ccc3ccc4ccccc4c3c2)cc(N(c2ccc(-c3ccc4c(c3)sc3c5ccccc5ccc43)cc2)c2ccc(-c3ccccc3-n3c4ccccc4c4ccccc43)cc2)c1]

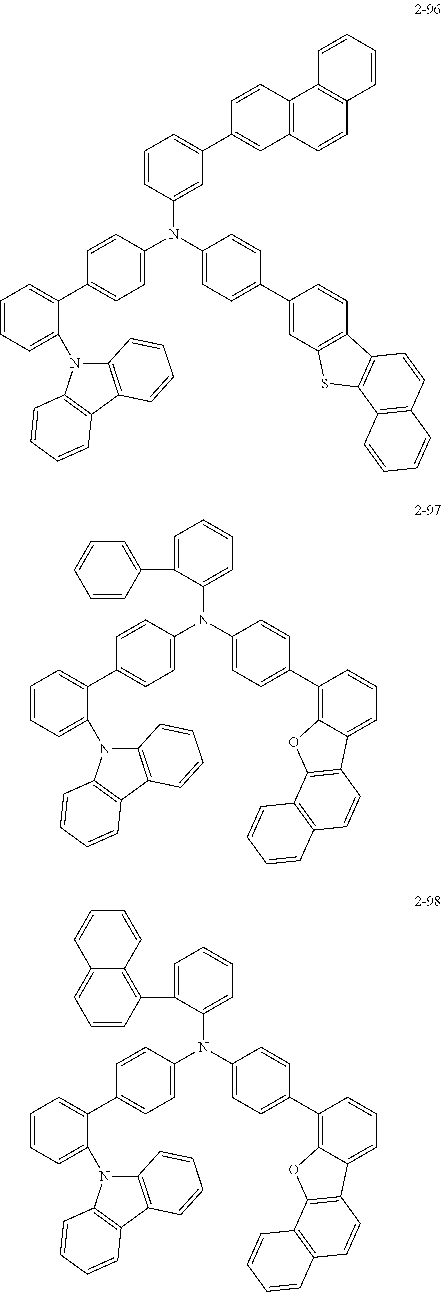 [SMILES: c1cc(-c2ccc3c(ccc4ccccc43)c2)cc(N(c2ccc(-c3ccc4c(c3)sc3c5ccccc5ccc43)cc2)c2ccc(-c3ccccc3-n3c4ccccc4c4ccccc43)cc2)c1 ; c1ccc(-c2ccccc2N(c2ccc(-c3ccccc3-n3c4ccccc4c4ccccc43)cc2)c2ccc(-c3cccc4c3oc3c5ccccc5ccc43)cc2)cc1 ; c1ccc(N(c2ccc(-c3ccccc3-n3c4ccccc4c4ccccc43)cc2)c2ccc(-c3cccc4c3oc3c5ccccc5ccc43)cc2)c(-c2cccc3ccccc23)c1]

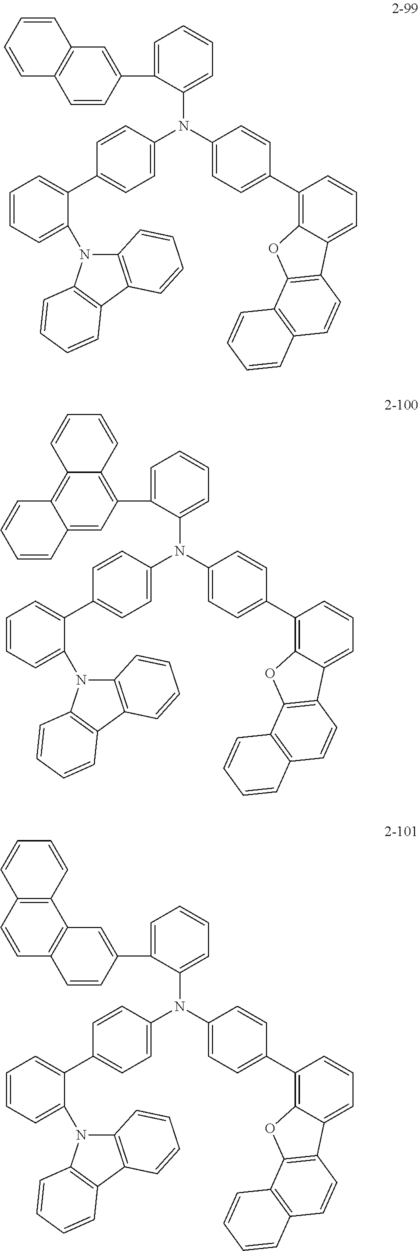 [SMILES: c1ccc(N(c2ccc(-c3ccccc3-n3c4ccccc4c4ccccc43)cc2)c2ccc(-c3cccc4c3oc3c5ccccc5ccc43)cc2)c(-c2ccc3ccccc3c2)c1 ; c1ccc(N(c2ccc(-c3ccccc3-n3c4ccccc4c4ccccc43)cc2)c2ccc(-c3cccc4c3oc3c5ccccc5ccc43)cc2)c(-c2cc3ccccc3c3ccccc23)c1 ; c1ccc(N(c2ccc(-c3ccccc3-n3c4ccccc4c4ccccc43)cc2)c2ccc(-c3cccc4c3oc3c5ccccc5ccc43)cc2)c(-c2ccc3ccc4ccccc4c3c2)c1]

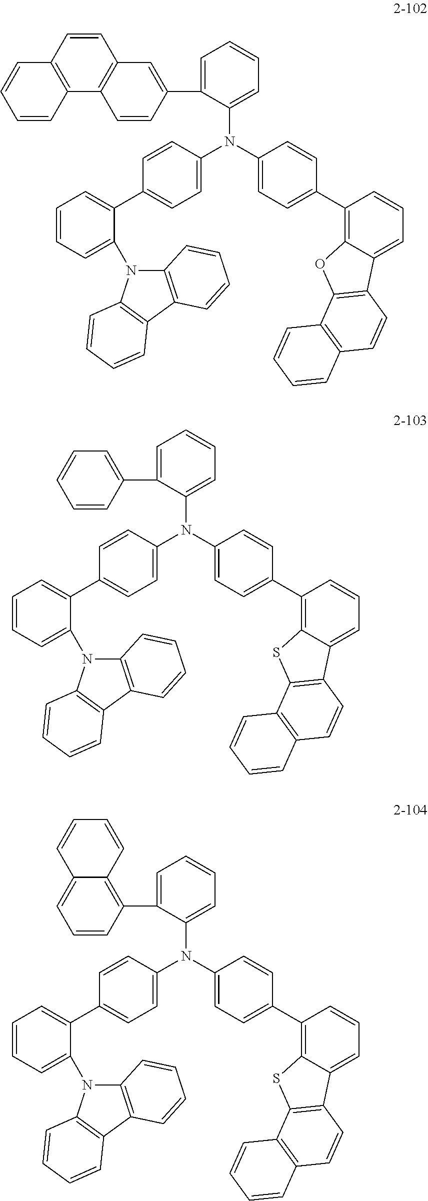 [SMILES: c1ccc(N(c2ccc(-c3ccccc3-n3c4ccccc4c4ccccc43)cc2)c2ccc(-c3cccc4c3oc3c5ccccc5ccc43)cc2)c(-c2ccc3c(ccc4ccccc43)c2)c1 ; c1ccc(-c2ccccc2N(c2ccc(-c3ccccc3-n3c4ccccc4c4ccccc43)cc2)c2ccc(-c3cccc4c3sc3c5ccccc5ccc43)cc2)cc1 ; c1ccc(N(c2ccc(-c3ccccc3-n3c4ccccc4c4ccccc43)cc2)c2ccc(-c3cccc4c3sc3c5ccccc5ccc43)cc2)c(-c2cccc3ccccc23)c1]

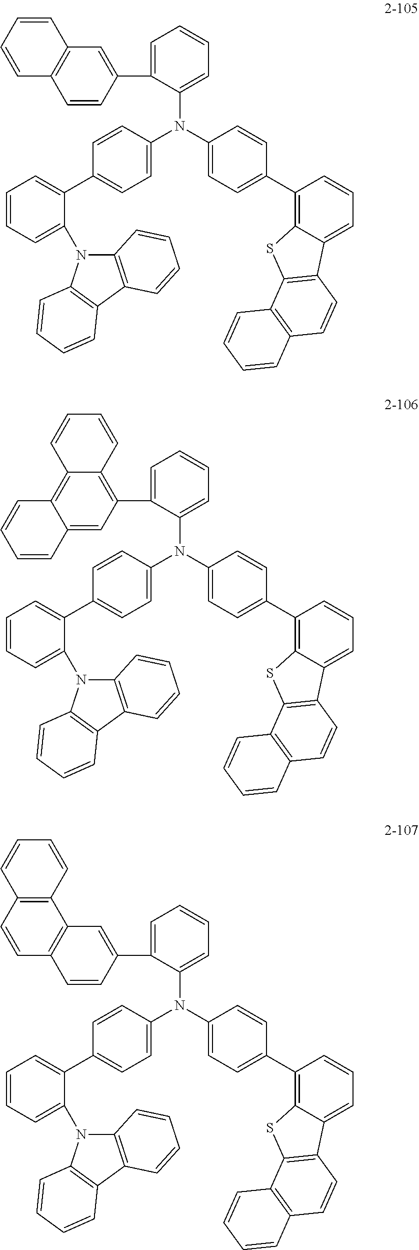 [SMILES: c1ccc(N(c2ccc(-c3ccccc3-n3c4ccccc4c4ccccc43)cc2)c2ccc(-c3cccc4c3sc3c5ccccc5ccc43)cc2)c(-c2ccc3ccccc3c2)c1 ; c1ccc(N(c2ccc(-c3ccccc3-n3c4ccccc4c4ccccc43)cc2)c2ccc(-c3cccc4c3sc3c5ccccc5ccc43)cc2)c(-c2cc3ccccc3c3ccccc23)c1 ; c1ccc(N(c2ccc(-c3ccccc3-n3c4ccccc4c4ccccc43)cc2)c2ccc(-c3cccc4c3sc3c5ccccc5ccc43)cc2)c(-c2ccc3ccc4ccccc4c3c2)c1]

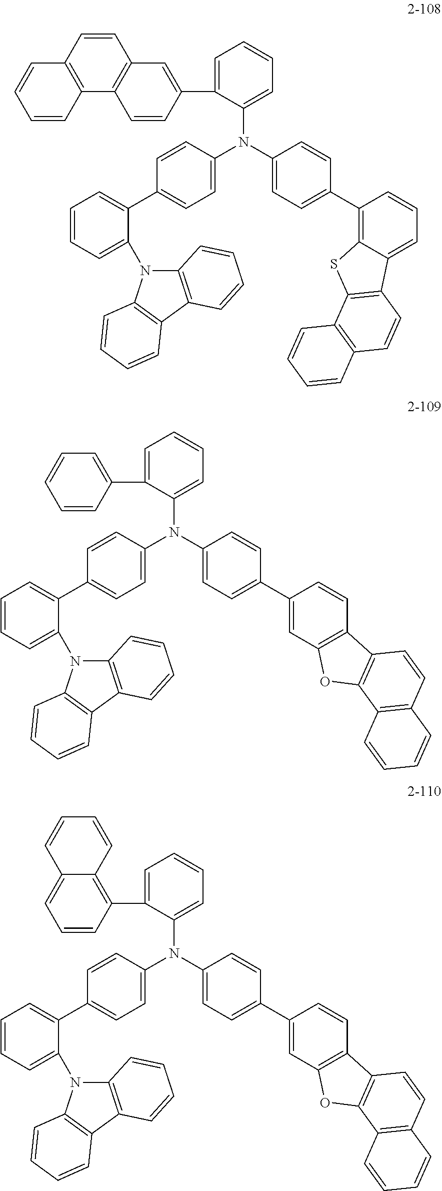 [SMILES: c1ccc(N(c2ccc(-c3ccccc3-n3c4ccccc4c4ccccc43)cc2)c2ccc(-c3cccc4c3sc3c5ccccc5ccc43)cc2)c(-c2ccc3c(ccc4ccccc43)c2)c1 ; c1ccc(-c2ccccc2N(c2ccc(-c3ccc4c(c3)oc3c5ccccc5ccc43)cc2)c2ccc(-c3ccccc3-n3c4ccccc4c4ccccc43)cc2)cc1 ; c1ccc(N(c2ccc(-c3ccc4c(c3)oc3c5ccccc5ccc43)cc2)c2ccc(-c3ccccc3-n3c4ccccc4c4ccccc43)cc2)c(-c2cccc3ccccc23)c1]

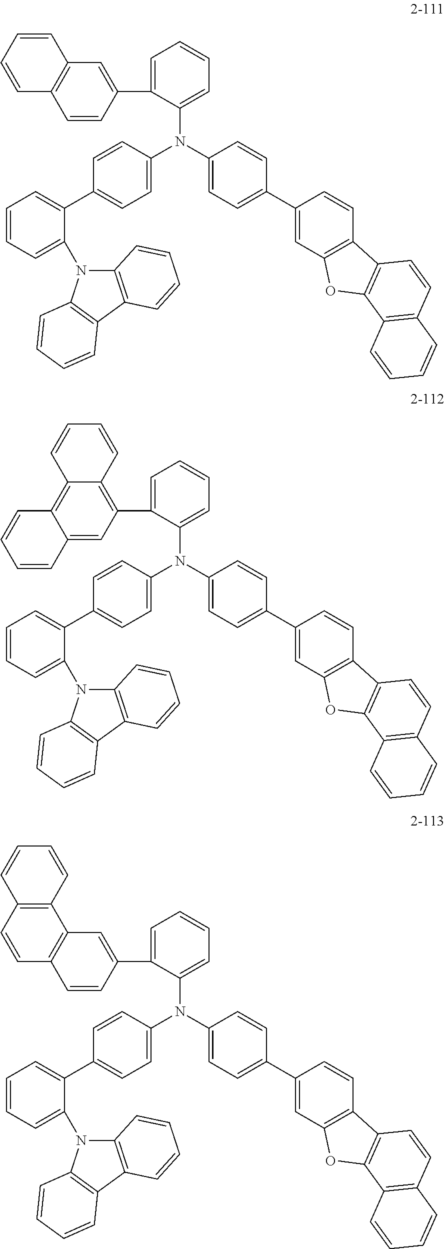 [SMILES: c1ccc(N(c2ccc(-c3ccc4c(c3)oc3c5ccccc5ccc43)cc2)c2ccc(-c3ccccc3-n3c4ccccc4c4ccccc43)cc2)c(-c2ccc3ccccc3c2)c1 ; c1ccc(N(c2ccc(-c3ccc4c(c3)oc3c5ccccc5ccc43)cc2)c2ccc(-c3ccccc3-n3c4ccccc4c4ccccc43)cc2)c(-c2cc3ccccc3c3ccccc23)c1 ; c1ccc(N(c2ccc(-c3ccc4c(c3)oc3c5ccccc5ccc43)cc2)c2ccc(-c3ccccc3-n3c4ccccc4c4ccccc43)cc2)c(-c2ccc3ccc4ccccc4c3c2)c1]

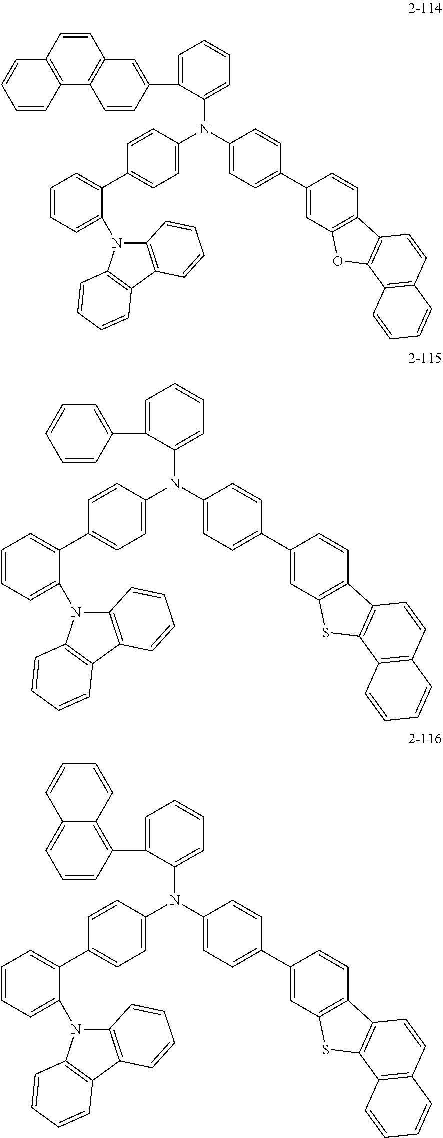 [SMILES: c1ccc(N(c2ccc(-c3ccc4c(c3)oc3c5ccccc5ccc43)cc2)c2ccc(-c3ccccc3-n3c4ccccc4c4ccccc43)cc2)c(-c2ccc3c(ccc4ccccc43)c2)c1 ; c1ccc(-c2ccccc2N(c2ccc(-c3ccc4c(c3)sc3c5ccccc5ccc43)cc2)c2ccc(-c3ccccc3-n3c4ccccc4c4ccccc43)cc2)cc1 ; c1ccc(N(c2ccc(-c3ccc4c(c3)sc3c5ccccc5ccc43)cc2)c2ccc(-c3ccccc3-n3c4ccccc4c4ccccc43)cc2)c(-c2cccc3ccccc23)c1]

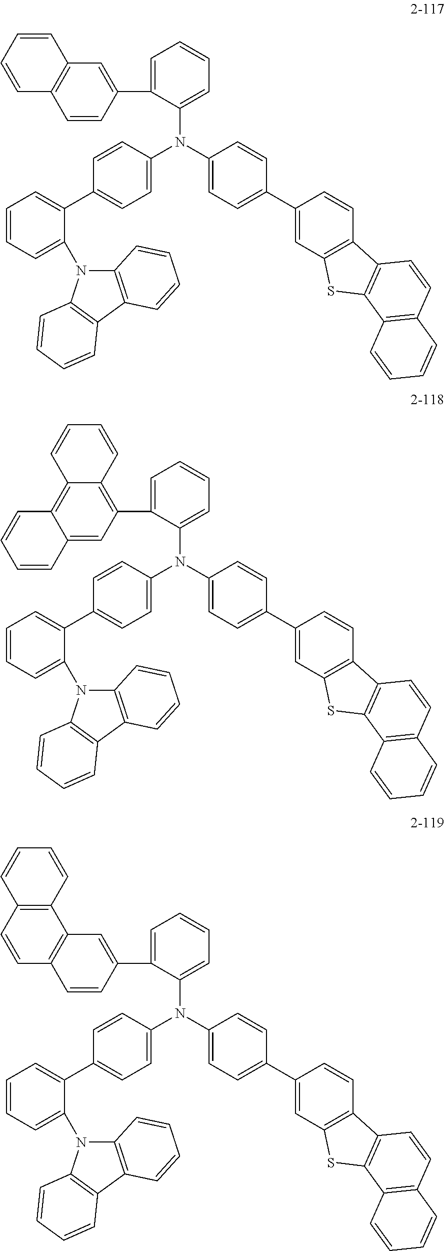 [SMILES: c1ccc(N(c2ccc(-c3ccc4c(c3)sc3c5ccccc5ccc43)cc2)c2ccc(-c3ccccc3-n3c4ccccc4c4ccccc43)cc2)c(-c2ccc3ccccc3c2)c1 ; c1ccc(N(c2ccc(-c3ccc4c(c3)sc3c5ccccc5ccc43)cc2)c2ccc(-c3ccccc3-n3c4ccccc4c4ccccc43)cc2)c(-c2cc3ccccc3c3ccccc23)c1 ; c1ccc(N(c2ccc(-c3ccc4c(c3)sc3c5ccccc5ccc43)cc2)c2ccc(-c3ccccc3-n3c4ccccc4c4ccccc43)cc2)c(-c2ccc3ccc4ccccc4c3c2)c1]

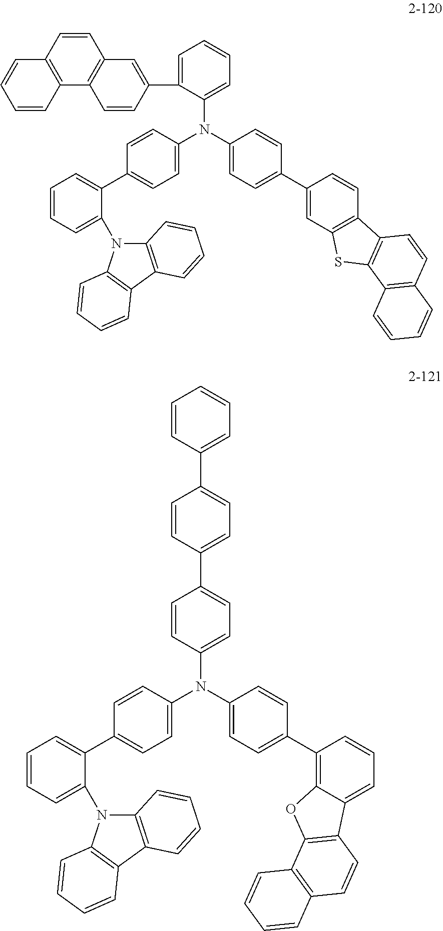 [SMILES: c1ccc(N(c2ccc(-c3ccc4c(c3)sc3c5ccccc5ccc43)cc2)c2ccc(-c3ccccc3-n3c4ccccc4c4ccccc43)cc2)c(-c2ccc3c(ccc4ccccc43)c2)c1 ; c1ccc(-c2ccc(-c3ccc(N(c4ccc(-c5ccccc5-n5c6ccccc6c6ccccc65)cc4)c4ccc(-c5cccc6c5oc5c7ccccc7ccc65)cc4)cc3)cc2)cc1]

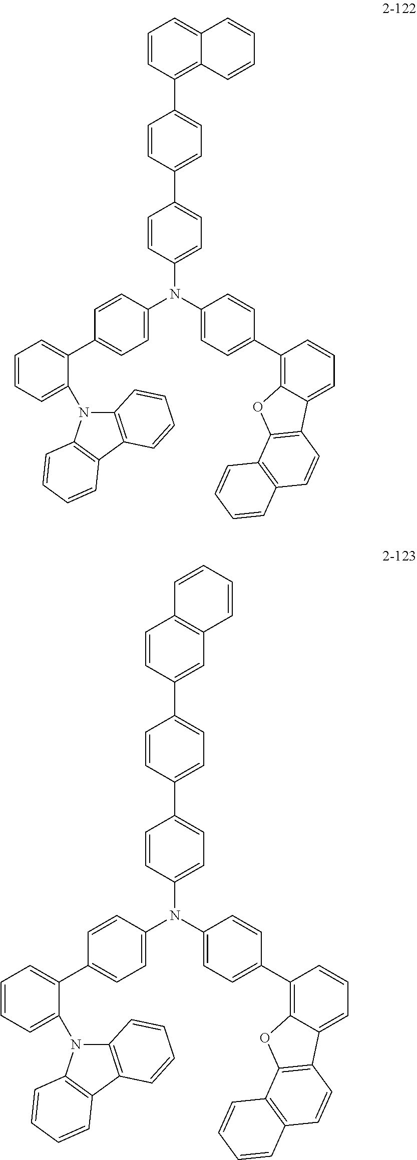 [SMILES: c1ccc(-n2c3ccccc3c3ccccc32)c(-c2ccc(N(c3ccc(-c4ccc(-c5cccc6ccccc56)cc4)cc3)c3ccc(-c4cccc5c4oc4c6ccccc6ccc54)cc3)cc2)c1 ; c1ccc(-n2c3ccccc3c3ccccc32)c(-c2ccc(N(c3ccc(-c4ccc(-c5ccc6ccccc6c5)cc4)cc3)c3ccc(-c4cccc5c4oc4c6ccccc6ccc54)cc3)cc2)c1]

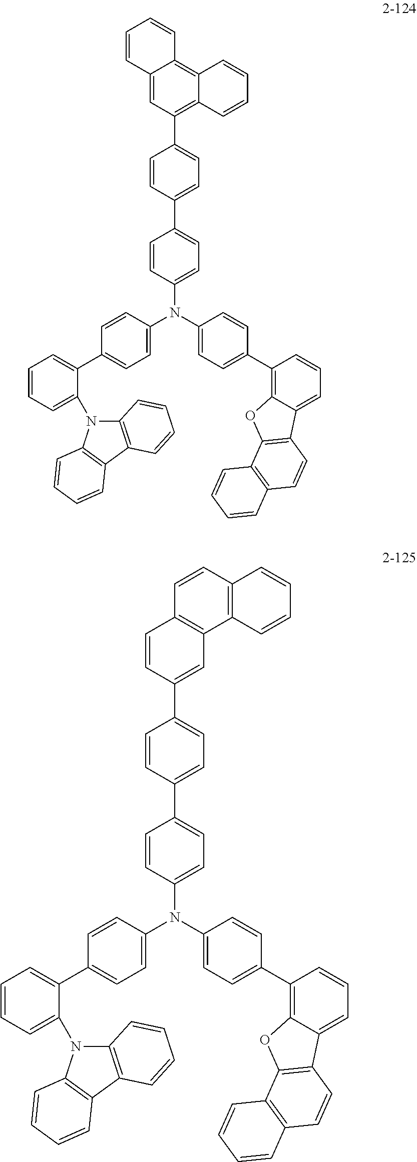 [SMILES: c1ccc(-n2c3ccccc3c3ccccc32)c(-c2ccc(N(c3ccc(-c4ccc(-c5cc6ccccc6c6ccccc56)cc4)cc3)c3ccc(-c4cccc5c4oc4c6ccccc6ccc54)cc3)cc2)c1 ; c1ccc(-n2c3ccccc3c3ccccc32)c(-c2ccc(N(c3ccc(-c4ccc(-c5ccc6ccc7ccccc7c6c5)cc4)cc3)c3ccc(-c4cccc5c4oc4c6ccccc6ccc54)cc3)cc2)c1]

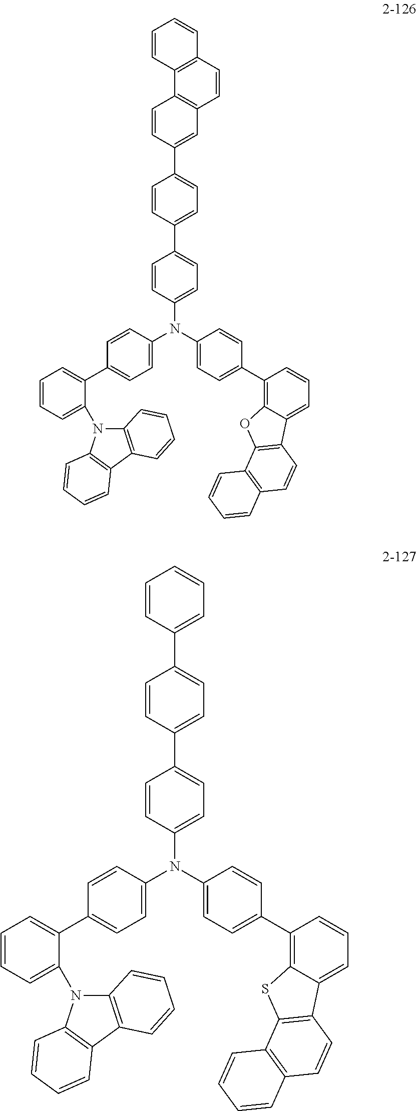 [SMILES: c1ccc(-n2c3ccccc3c3ccccc32)c(-c2ccc(N(c3ccc(-c4ccc(-c5ccc6c(ccc7ccccc76)c5)cc4)cc3)c3ccc(-c4cccc5c4oc4c6ccccc6ccc54)cc3)cc2)c1 ; c1ccc(-c2ccc(-c3ccc(N(c4ccc(-c5ccccc5-n5c6ccccc6c6ccccc65)cc4)c4ccc(-c5cccc6c5sc5c7ccccc7ccc65)cc4)cc3)cc2)cc1]

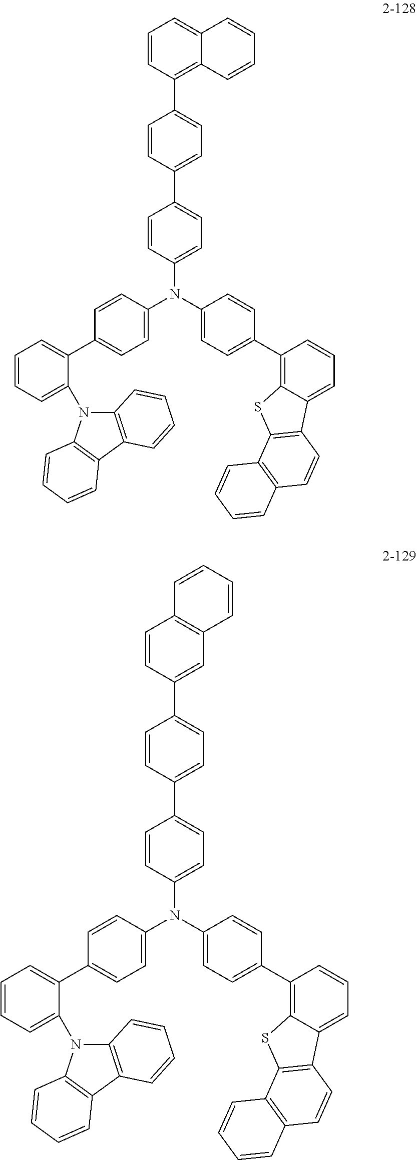 [SMILES: c1ccc(-n2c3ccccc3c3ccccc32)c(-c2ccc(N(c3ccc(-c4ccc(-c5cccc6ccccc56)cc4)cc3)c3ccc(-c4cccc5c4sc4c6ccccc6ccc54)cc3)cc2)c1 ; c1ccc(-n2c3ccccc3c3ccccc32)c(-c2ccc(N(c3ccc(-c4ccc(-c5ccc6ccccc6c5)cc4)cc3)c3ccc(-c4cccc5c4sc4c6ccccc6ccc54)cc3)cc2)c1]

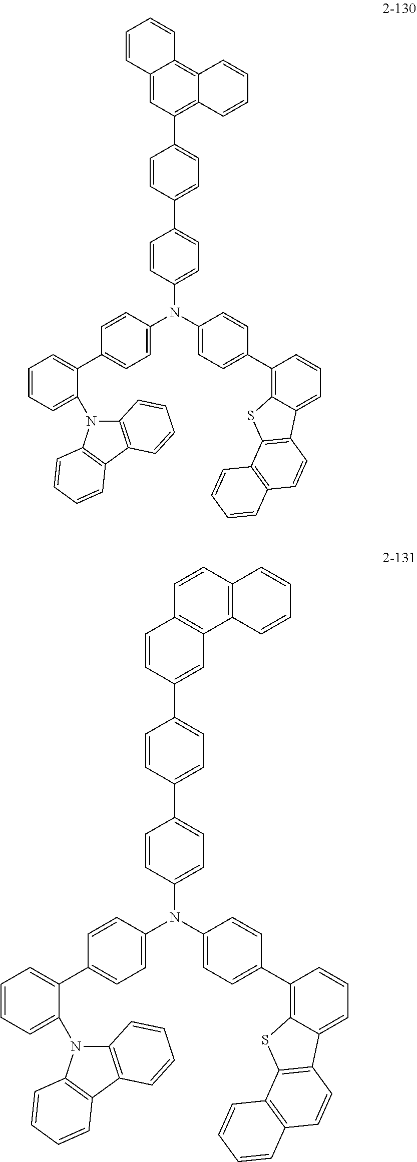 [SMILES: c1ccc(-n2c3ccccc3c3ccccc32)c(-c2ccc(N(c3ccc(-c4ccc(-c5cc6ccccc6c6ccccc56)cc4)cc3)c3ccc(-c4cccc5c4sc4c6ccccc6ccc54)cc3)cc2)c1 ; c1ccc(-n2c3ccccc3c3ccccc32)c(-c2ccc(N(c3ccc(-c4ccc(-c5ccc6ccc7ccccc7c6c5)cc4)cc3)c3ccc(-c4cccc5c4sc4c6ccccc6ccc54)cc3)cc2)c1]

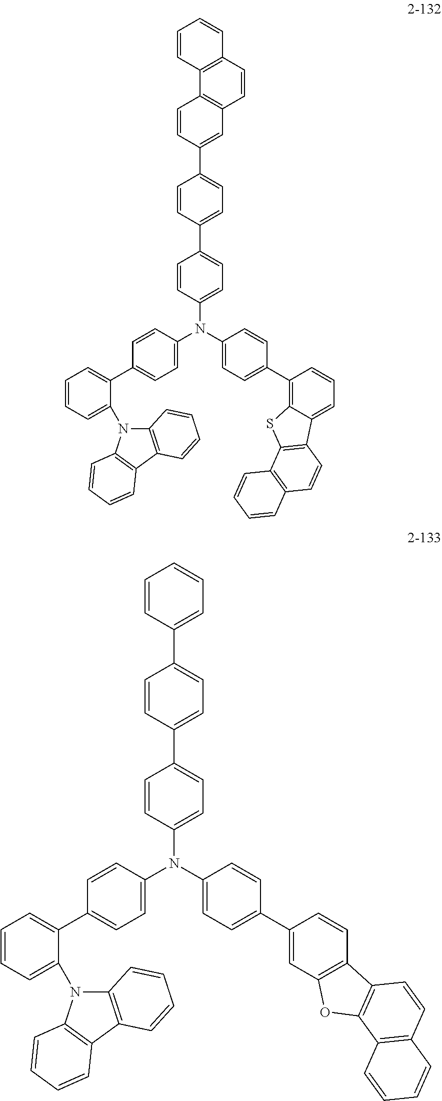 [SMILES: c1ccc(-n2c3ccccc3c3ccccc32)c(-c2ccc(N(c3ccc(-c4ccc(-c5ccc6c(ccc7ccccc76)c5)cc4)cc3)c3ccc(-c4cccc5c4sc4c6ccccc6ccc54)cc3)cc2)c1 ; c1ccc(-c2ccc(-c3ccc(N(c4ccc(-c5ccc6c(c5)oc5c7ccccc7ccc65)cc4)c4ccc(-c5ccccc5-n5c6ccccc6c6ccccc65)cc4)cc3)cc2)cc1]

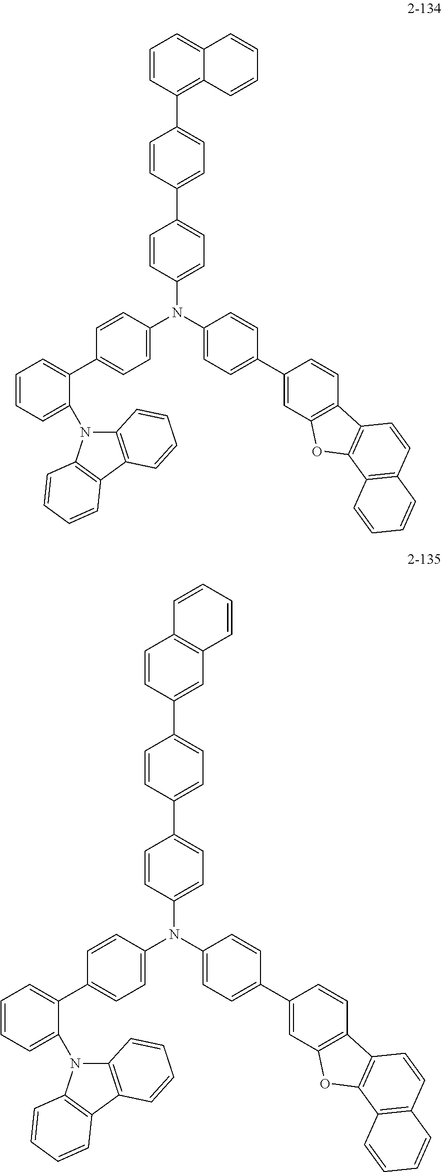 [SMILES: c1ccc(-n2c3ccccc3c3ccccc32)c(-c2ccc(N(c3ccc(-c4ccc(-c5cccc6ccccc56)cc4)cc3)c3ccc(-c4ccc5c(c4)oc4c6ccccc6ccc54)cc3)cc2)c1 ; c1ccc(-n2c3ccccc3c3ccccc32)c(-c2ccc(N(c3ccc(-c4ccc(-c5ccc6ccccc6c5)cc4)cc3)c3ccc(-c4ccc5c(c4)oc4c6ccccc6ccc54)cc3)cc2)c1]

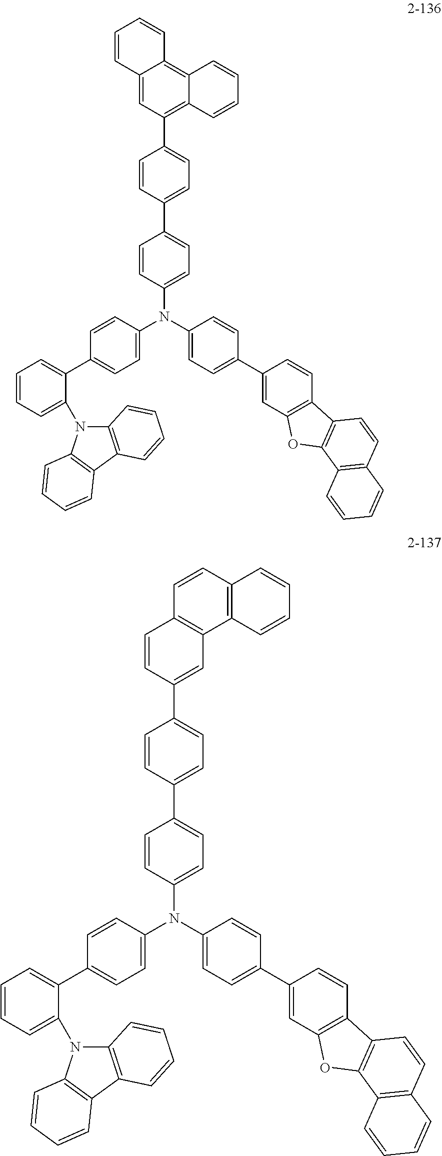 [SMILES: c1ccc(-n2c3ccccc3c3ccccc32)c(-c2ccc(N(c3ccc(-c4ccc(-c5cc6ccccc6c6ccccc56)cc4)cc3)c3ccc(-c4ccc5c(c4)oc4c6ccccc6ccc54)cc3)cc2)c1 ; c1ccc(-n2c3ccccc3c3ccccc32)c(-c2ccc(N(c3ccc(-c4ccc(-c5ccc6ccc7ccccc7c6c5)cc4)cc3)c3ccc(-c4ccc5c(c4)oc4c6ccccc6ccc54)cc3)cc2)c1]

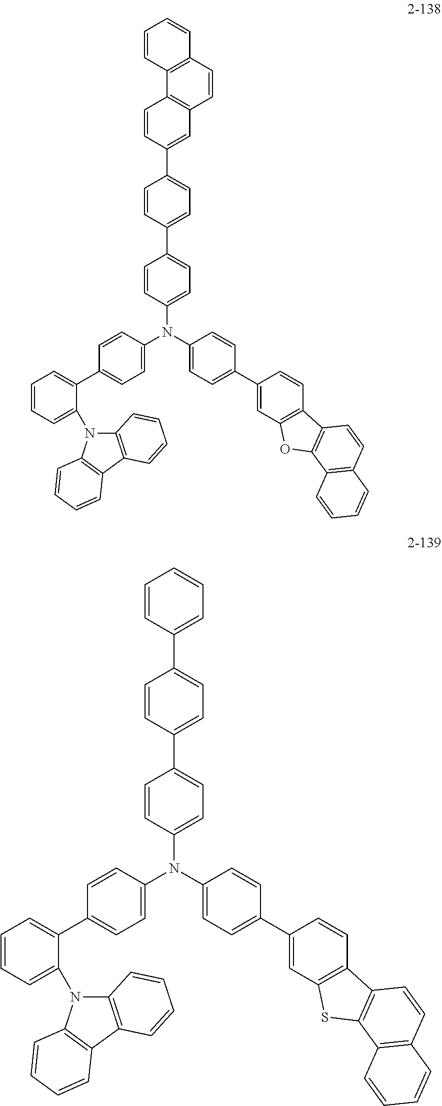 [SMILES: c1ccc(-n2c3ccccc3c3ccccc32)c(-c2ccc(N(c3ccc(-c4ccc(-c5ccc6c(ccc7ccccc76)c5)cc4)cc3)c3ccc(-c4ccc5c(c4)oc4c6ccccc6ccc54)cc3)cc2)c1 ; c1ccc(-c2ccc(-c3ccc(N(c4ccc(-c5ccc6c(c5)sc5c7ccccc7ccc65)cc4)c4ccc(-c5ccccc5-n5c6ccccc6c6ccccc65)cc4)cc3)cc2)cc1]

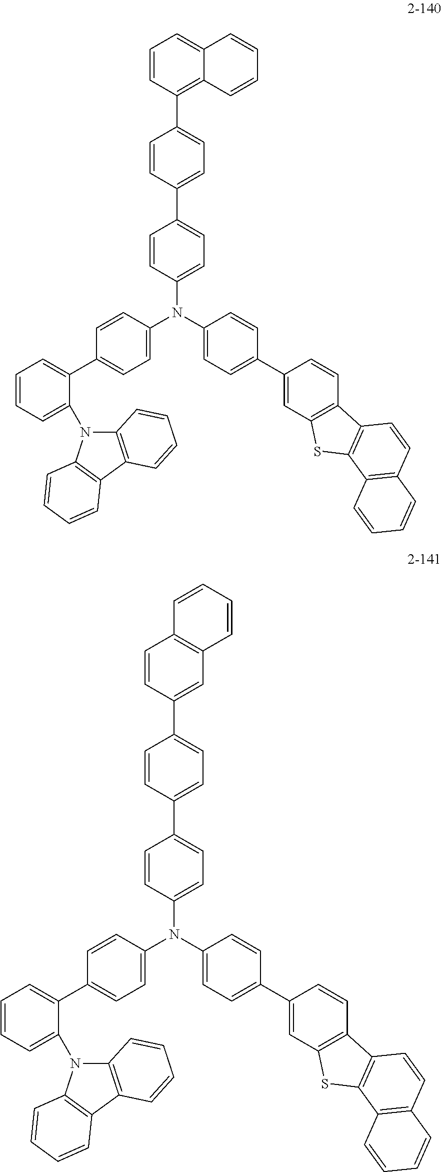 [SMILES: c1ccc(-n2c3ccccc3c3ccccc32)c(-c2ccc(N(c3ccc(-c4ccc(-c5cccc6ccccc56)cc4)cc3)c3ccc(-c4ccc5c(c4)sc4c6ccccc6ccc54)cc3)cc2)c1 ; c1ccc(-n2c3ccccc3c3ccccc32)c(-c2ccc(N(c3ccc(-c4ccc(-c5ccc6ccccc6c5)cc4)cc3)c3ccc(-c4ccc5c(c4)sc4c6ccccc6ccc54)cc3)cc2)c1]

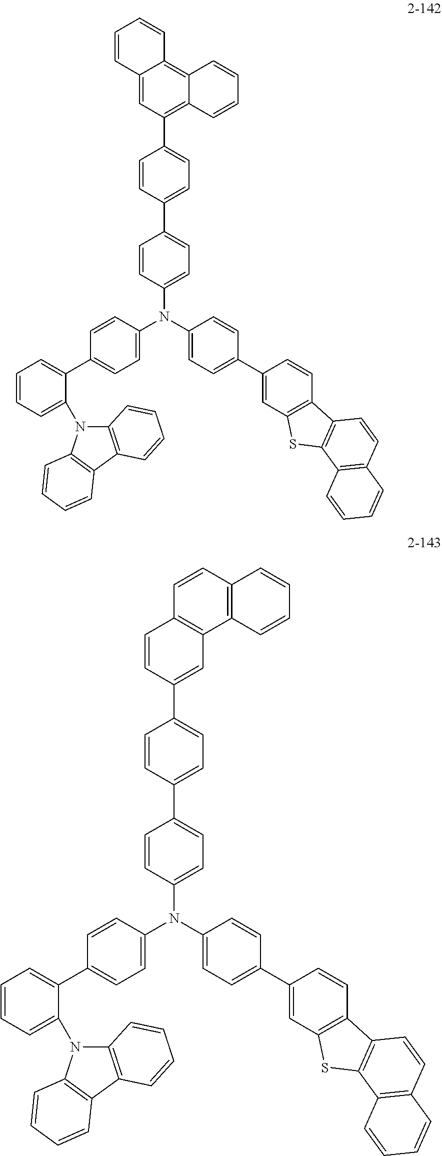 [SMILES: c1ccc(-n2c3ccccc3c3ccccc32)c(-c2ccc(N(c3ccc(-c4ccc(-c5cc6ccccc6c6ccccc56)cc4)cc3)c3ccc(-c4ccc5c(c4)sc4c6ccccc6ccc54)cc3)cc2)c1 ; c1ccc(-n2c3ccccc3c3ccccc32)c(-c2ccc(N(c3ccc(-c4ccc(-c5ccc6ccc7ccccc7c6c5)cc4)cc3)c3ccc(-c4ccc5c(c4)sc4c6ccccc6ccc54)cc3)cc2)c1]

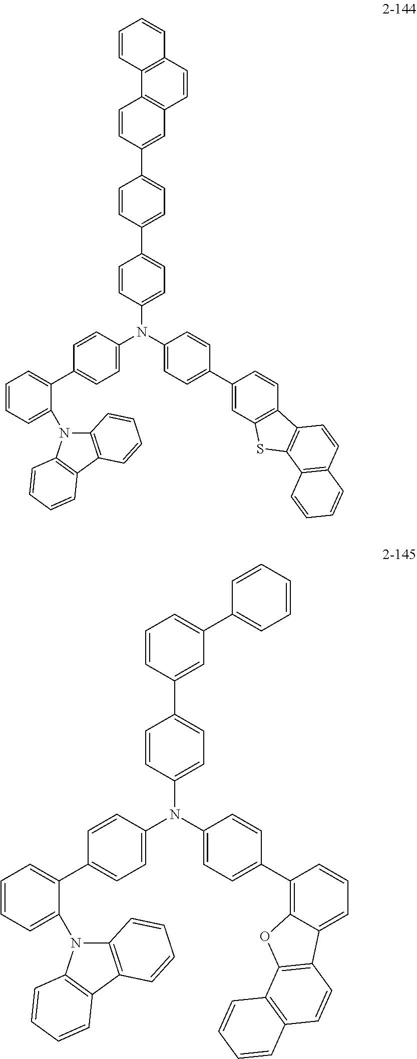 [SMILES: c1ccc(-n2c3ccccc3c3ccccc32)c(-c2ccc(N(c3ccc(-c4ccc(-c5ccc6c(ccc7ccccc76)c5)cc4)cc3)c3ccc(-c4ccc5c(c4)sc4c6ccccc6ccc54)cc3)cc2)c1 ; c1ccc(-c2cccc(-c3ccc(N(c4ccc(-c5ccccc5-n5c6ccccc6c6ccccc65)cc4)c4ccc(-c5cccc6c5oc5c7ccccc7ccc65)cc4)cc3)c2)cc1]

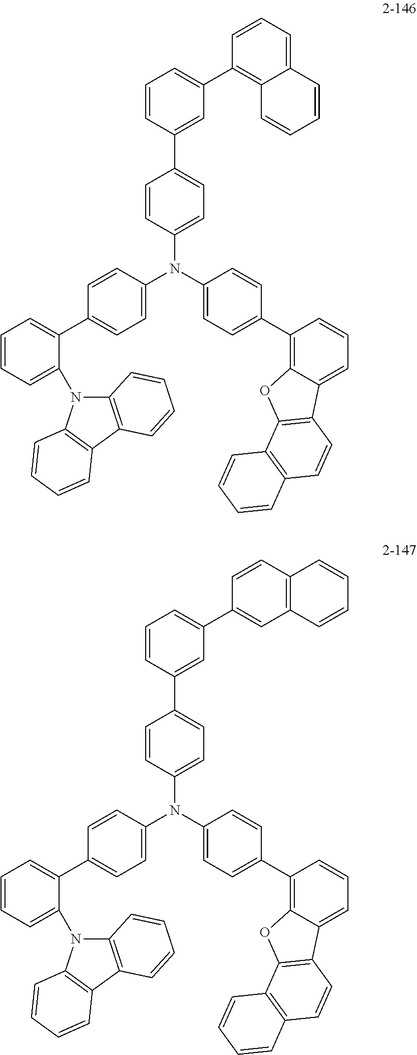 [SMILES: c1cc(-c2ccc(N(c3ccc(-c4ccccc4-n4c5ccccc5c5ccccc54)cc3)c3ccc(-c4cccc5c4oc4c6ccccc6ccc54)cc3)cc2)cc(-c2cccc3ccccc23)c1 ; c1cc(-c2ccc(N(c3ccc(-c4ccccc4-n4c5ccccc5c5ccccc54)cc3)c3ccc(-c4cccc5c4oc4c6ccccc6ccc54)cc3)cc2)cc(-c2ccc3ccccc3c2)c1]

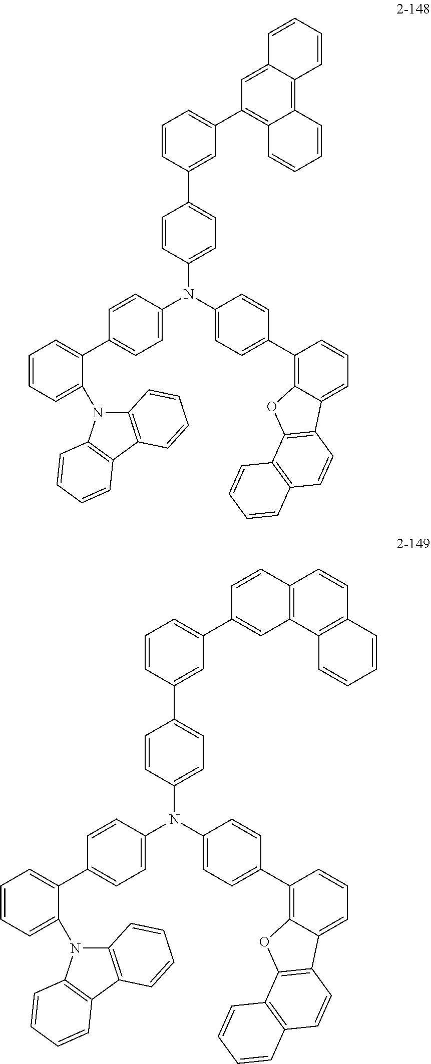 [SMILES: c1cc(-c2ccc(N(c3ccc(-c4ccccc4-n4c5ccccc5c5ccccc54)cc3)c3ccc(-c4cccc5c4oc4c6ccccc6ccc54)cc3)cc2)cc(-c2cc3ccccc3c3ccccc23)c1 ; c1cc(-c2ccc(N(c3ccc(-c4ccccc4-n4c5ccccc5c5ccccc54)cc3)c3ccc(-c4cccc5c4oc4c6ccccc6ccc54)cc3)cc2)cc(-c2ccc3ccc4ccccc4c3c2)c1]

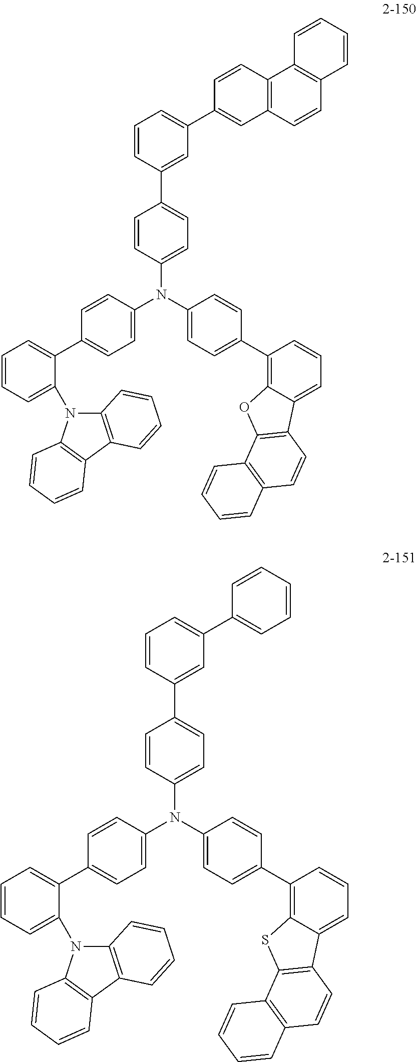 [SMILES: c1cc(-c2ccc(N(c3ccc(-c4ccccc4-n4c5ccccc5c5ccccc54)cc3)c3ccc(-c4cccc5c4oc4c6ccccc6ccc54)cc3)cc2)cc(-c2ccc3c(ccc4ccccc43)c2)c1 ; c1ccc(-c2cccc(-c3ccc(N(c4ccc(-c5ccccc5-n5c6ccccc6c6ccccc65)cc4)c4ccc(-c5cccc6c5sc5c7ccccc7ccc65)cc4)cc3)c2)cc1]

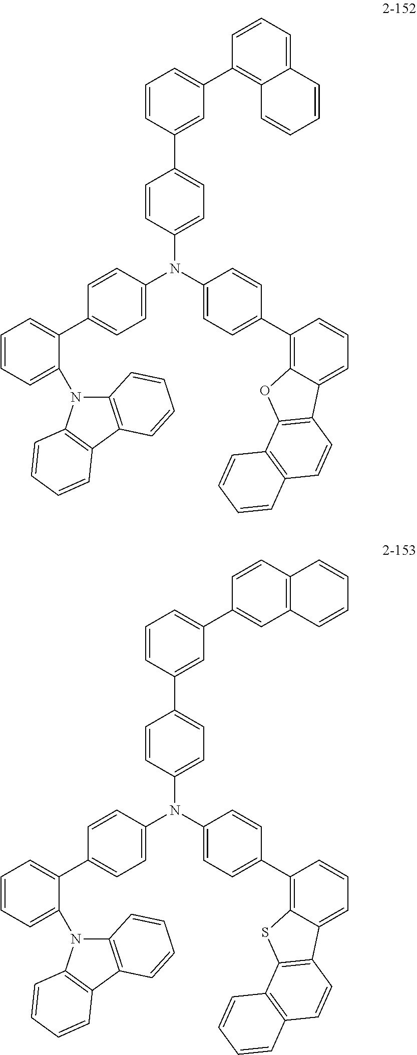 [SMILES: c1cc(-c2ccc(N(c3ccc(-c4ccccc4-n4c5ccccc5c5ccccc54)cc3)c3ccc(-c4cccc5c4oc4c6ccccc6ccc54)cc3)cc2)cc(-c2cccc3ccccc23)c1 ; c1cc(-c2ccc(N(c3ccc(-c4ccccc4-n4c5ccccc5c5ccccc54)cc3)c3ccc(-c4cccc5c4sc4c6ccccc6ccc54)cc3)cc2)cc(-c2ccc3ccccc3c2)c1]

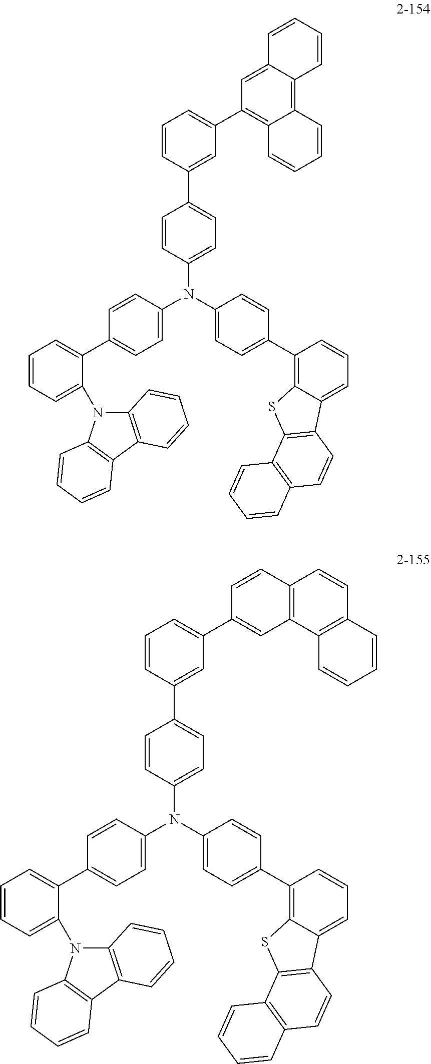 [SMILES: c1cc(-c2ccc(N(c3ccc(-c4ccccc4-n4c5ccccc5c5ccccc54)cc3)c3ccc(-c4cccc5c4sc4c6ccccc6ccc54)cc3)cc2)cc(-c2cc3ccccc3c3ccccc23)c1 ; c1cc(-c2ccc(N(c3ccc(-c4ccccc4-n4c5ccccc5c5ccccc54)cc3)c3ccc(-c4cccc5c4sc4c6ccccc6ccc54)cc3)cc2)cc(-c2ccc3ccc4ccccc4c3c2)c1]

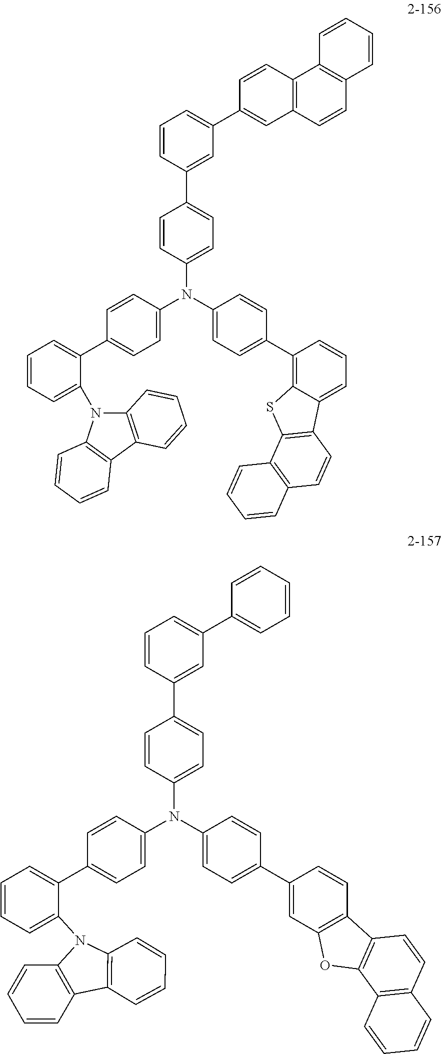 [SMILES: c1cc(-c2ccc(N(c3ccc(-c4ccccc4-n4c5ccccc5c5ccccc54)cc3)c3ccc(-c4cccc5c4sc4c6ccccc6ccc54)cc3)cc2)cc(-c2ccc3c(ccc4ccccc43)c2)c1 ; c1ccc(-c2cccc(-c3ccc(N(c4ccc(-c5ccc6c(c5)oc5c7ccccc7ccc65)cc4)c4ccc(-c5ccccc5-n5c6ccccc6c6ccccc65)cc4)cc3)c2)cc1]

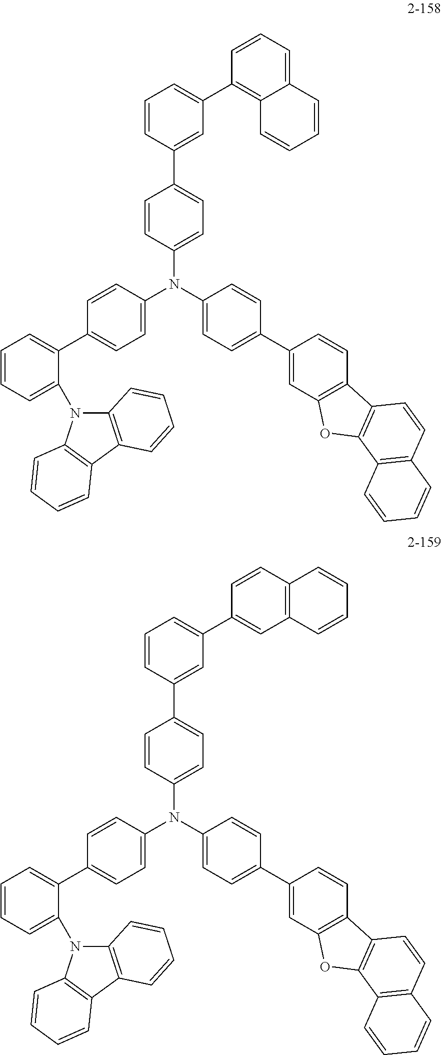 [SMILES: c1cc(-c2ccc(N(c3ccc(-c4ccc5c(c4)oc4c6ccccc6ccc54)cc3)c3ccc(-c4ccccc4-n4c5ccccc5c5ccccc54)cc3)cc2)cc(-c2cccc3ccccc23)c1 ; c1cc(-c2ccc(N(c3ccc(-c4ccc5c(c4)oc4c6ccccc6ccc54)cc3)c3ccc(-c4ccccc4-n4c5ccccc5c5ccccc54)cc3)cc2)cc(-c2ccc3ccccc3c2)c1]

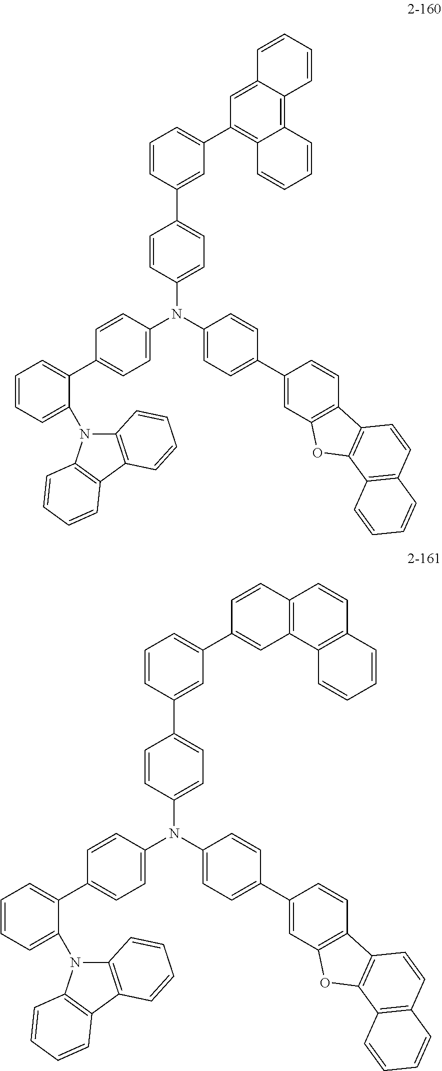 [SMILES: c1cc(-c2ccc(N(c3ccc(-c4ccc5c(c4)oc4c6ccccc6ccc54)cc3)c3ccc(-c4ccccc4-n4c5ccccc5c5ccccc54)cc3)cc2)cc(-c2cc3ccccc3c3ccccc23)c1 ; c1cc(-c2ccc(N(c3ccc(-c4ccc5c(c4)oc4c6ccccc6ccc54)cc3)c3ccc(-c4ccccc4-n4c5ccccc5c5ccccc54)cc3)cc2)cc(-c2ccc3ccc4ccccc4c3c2)c1]

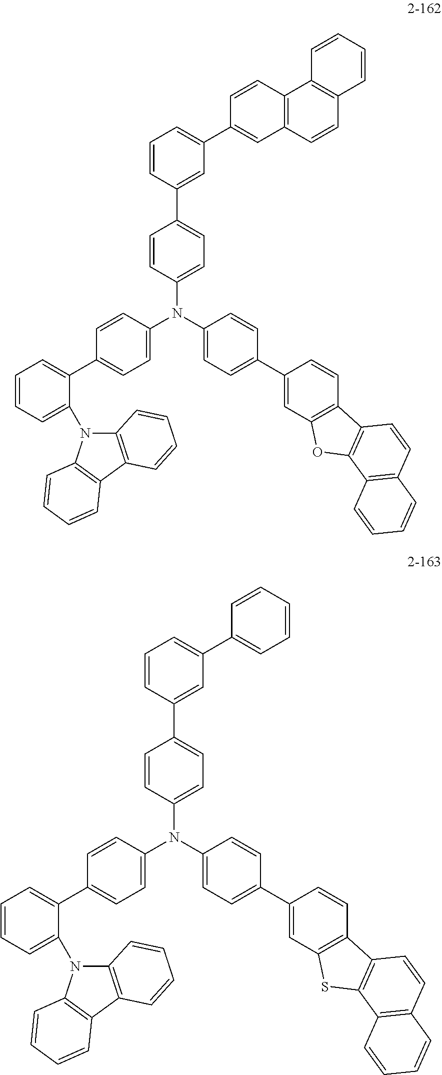 [SMILES: c1cc(-c2ccc(N(c3ccc(-c4ccc5c(c4)oc4c6ccccc6ccc54)cc3)c3ccc(-c4ccccc4-n4c5ccccc5c5ccccc54)cc3)cc2)cc(-c2ccc3c(ccc4ccccc43)c2)c1 ; c1ccc(-c2cccc(-c3ccc(N(c4ccc(-c5ccc6c(c5)sc5c7ccccc7ccc65)cc4)c4ccc(-c5ccccc5-n5c6ccccc6c6ccccc65)cc4)cc3)c2)cc1]

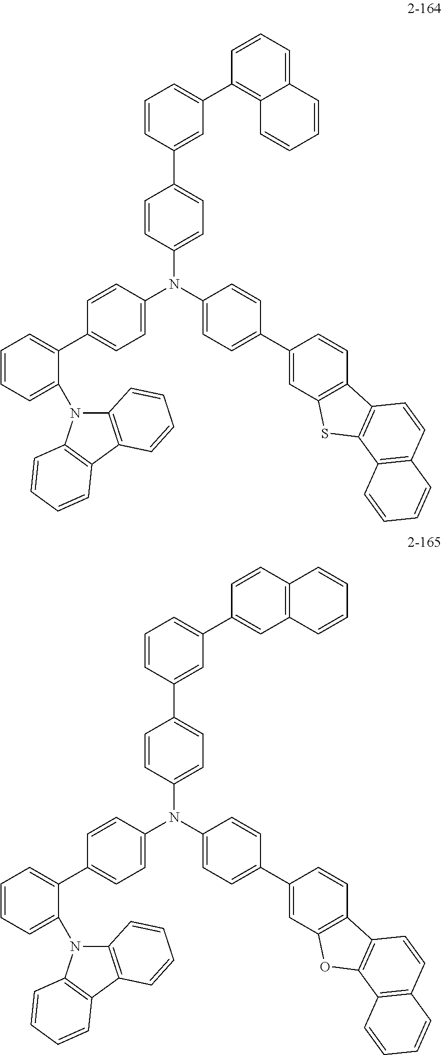 [SMILES: c1cc(-c2ccc(N(c3ccc(-c4ccc5c(c4)sc4c6ccccc6ccc54)cc3)c3ccc(-c4ccccc4-n4c5ccccc5c5ccccc54)cc3)cc2)cc(-c2cccc3ccccc23)c1 ; c1cc(-c2ccc(N(c3ccc(-c4ccc5c(c4)oc4c6ccccc6ccc54)cc3)c3ccc(-c4ccccc4-n4c5ccccc5c5ccccc54)cc3)cc2)cc(-c2ccc3ccccc3c2)c1]

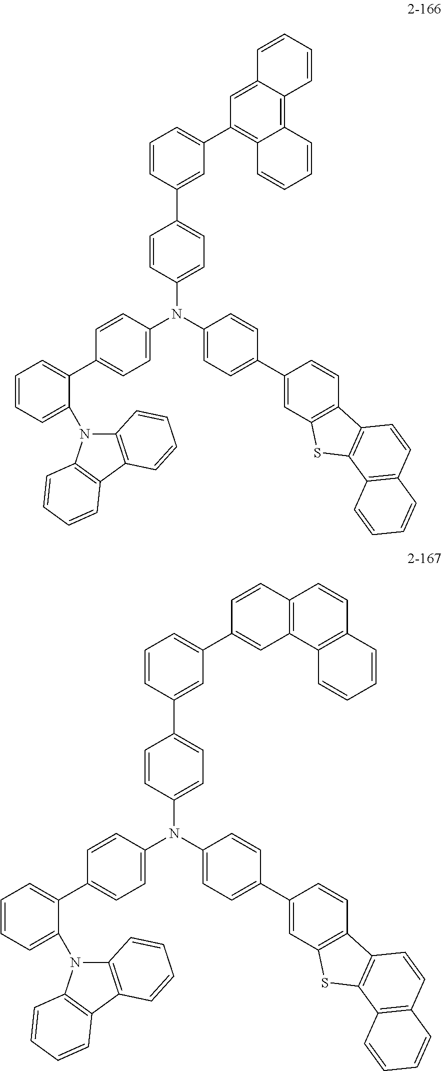 [SMILES: c1cc(-c2ccc(N(c3ccc(-c4ccc5c(c4)sc4c6ccccc6ccc54)cc3)c3ccc(-c4ccccc4-n4c5ccccc5c5ccccc54)cc3)cc2)cc(-c2cc3ccccc3c3ccccc23)c1 ; c1cc(-c2ccc(N(c3ccc(-c4ccc5c(c4)sc4c6ccccc6ccc54)cc3)c3ccc(-c4ccccc4-n4c5ccccc5c5ccccc54)cc3)cc2)cc(-c2ccc3ccc4ccccc4c3c2)c1]

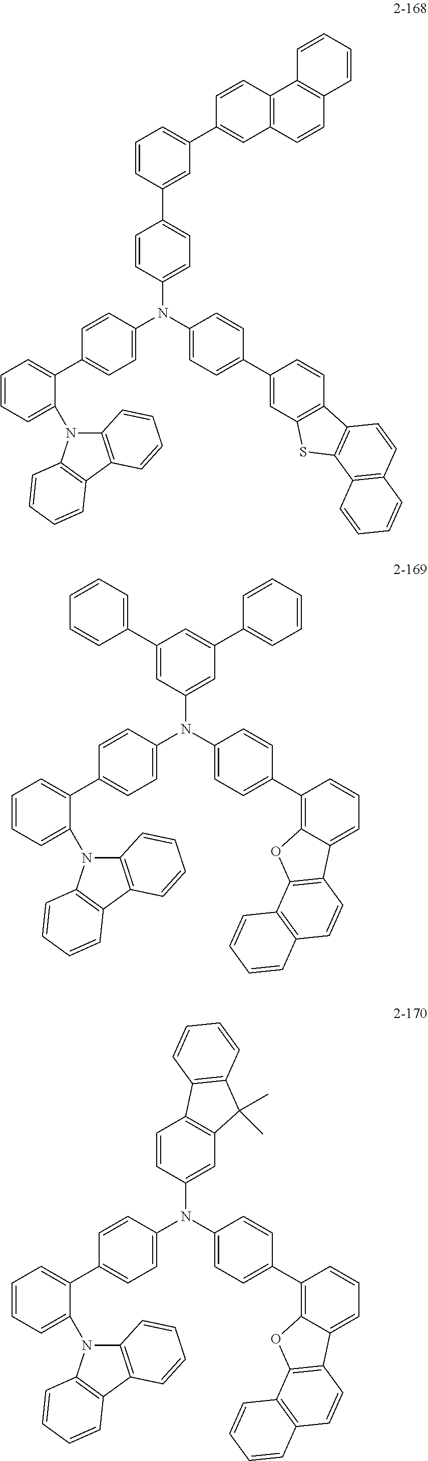 [SMILES: c1cc(-c2ccc(N(c3ccc(-c4ccc5c(c4)sc4c6ccccc6ccc54)cc3)c3ccc(-c4ccccc4-n4c5ccccc5c5ccccc54)cc3)cc2)cc(-c2ccc3c(ccc4ccccc43)c2)c1 ; c1ccc(-c2cc(-c3ccccc3)cc(N(c3ccc(-c4ccccc4-n4c5ccccc5c5ccccc54)cc3)c3ccc(-c4cccc5c4oc4c6ccccc6ccc54)cc3)c2)cc1 ; CC1(C)c2ccccc2-c2ccc(N(c3ccc(-c4ccccc4-n4c5ccccc5c5ccccc54)cc3)c3ccc(-c4cccc5c4oc4c6ccccc6ccc54)cc3)cc21]

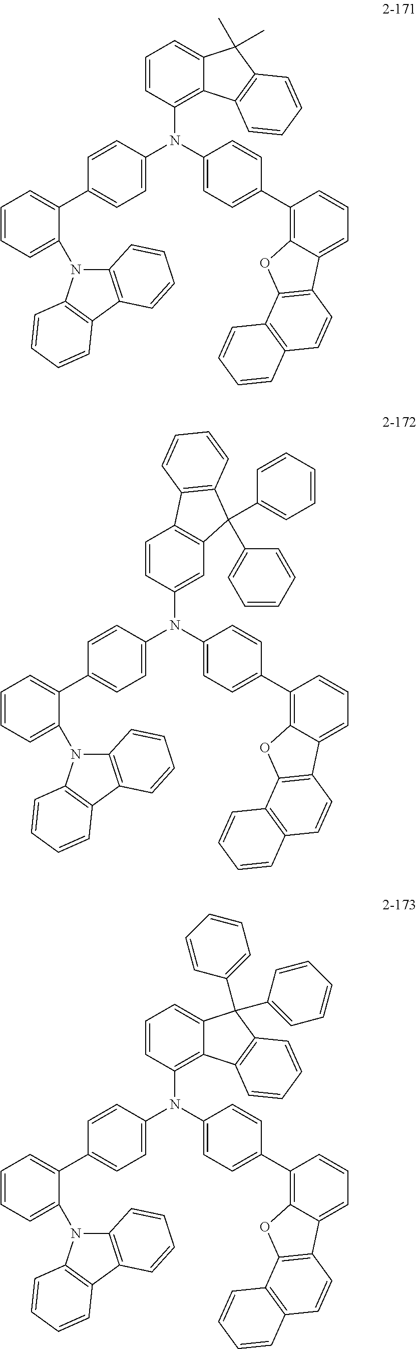 [SMILES: CC1(C)c2ccccc2-c2c(N(c3ccc(-c4ccccc4-n4c5ccccc5c5ccccc54)cc3)c3ccc(-c4cccc5c4oc4c6ccccc6ccc54)cc3)cccc21 ; c1ccc(C2(c3ccccc3)c3ccccc3-c3ccc(N(c4ccc(-c5ccccc5-n5c6ccccc6c6ccccc65)cc4)c4ccc(-c5cccc6c5oc5c7ccccc7ccc65)cc4)cc32)cc1 ; c1ccc(C2(c3ccccc3)c3ccccc3-c3c(N(c4ccc(-c5ccccc5-n5c6ccccc6c6ccccc65)cc4)c4ccc(-c5cccc6c5oc5c7ccccc7ccc65)cc4)cccc32)cc1]

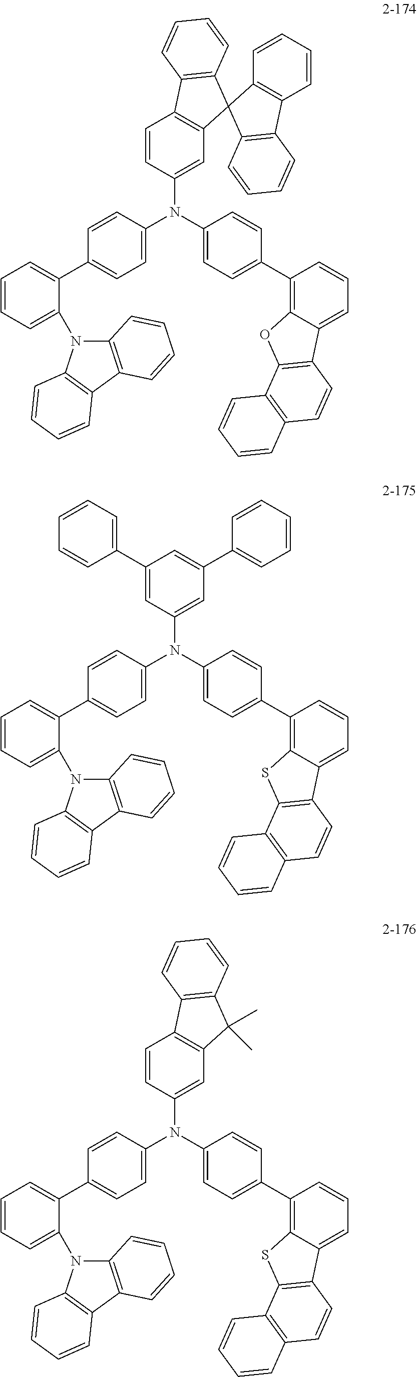 [SMILES: c1ccc(-n2c3ccccc3c3ccccc32)c(-c2ccc(N(c3ccc(-c4cccc5c4oc4c6ccccc6ccc54)cc3)c3ccc4c(c3)C3(c5ccccc5-c5ccccc53)c3ccccc3-4)cc2)c1 ; c1ccc(-c2cc(-c3ccccc3)cc(N(c3ccc(-c4ccccc4-n4c5ccccc5c5ccccc54)cc3)c3ccc(-c4cccc5c4sc4c6ccccc6ccc54)cc3)c2)cc1 ; CC1(C)c2ccccc2-c2ccc(N(c3ccc(-c4ccccc4-n4c5ccccc5c5ccccc54)cc3)c3ccc(-c4cccc5c4sc4c6ccccc6ccc54)cc3)cc21]

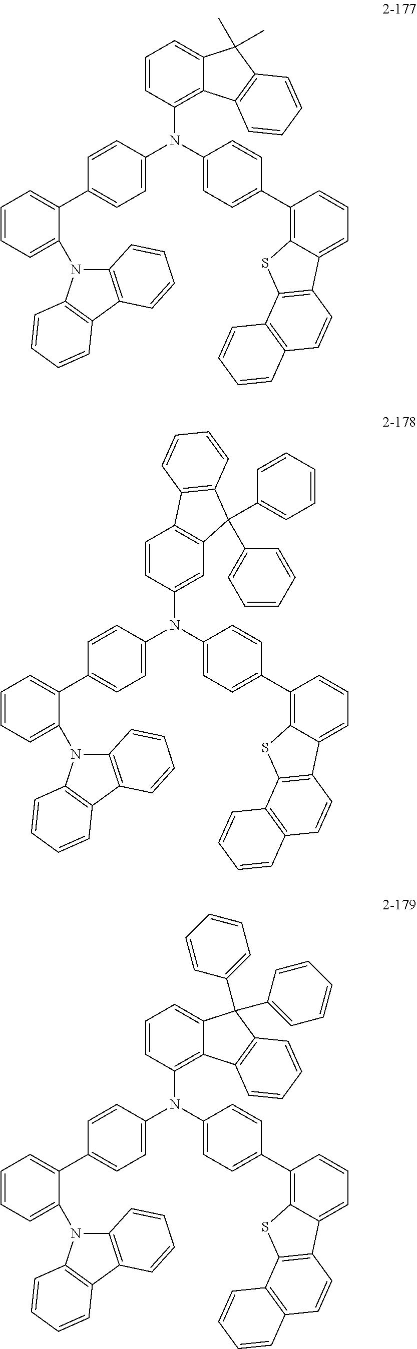 [SMILES: CC1(C)c2ccccc2-c2c(N(c3ccc(-c4ccccc4-n4c5ccccc5c5ccccc54)cc3)c3ccc(-c4cccc5c4sc4c6ccccc6ccc54)cc3)cccc21 ; c1ccc(C2(c3ccccc3)c3ccccc3-c3ccc(N(c4ccc(-c5ccccc5-n5c6ccccc6c6ccccc65)cc4)c4ccc(-c5cccc6c5sc5c7ccccc7ccc65)cc4)cc32)cc1 ; c1ccc(C2(c3ccccc3)c3ccccc3-c3c(N(c4ccc(-c5ccccc5-n5c6ccccc6c6ccccc65)cc4)c4ccc(-c5cccc6c5sc5c7ccccc7ccc65)cc4)cccc32)cc1]

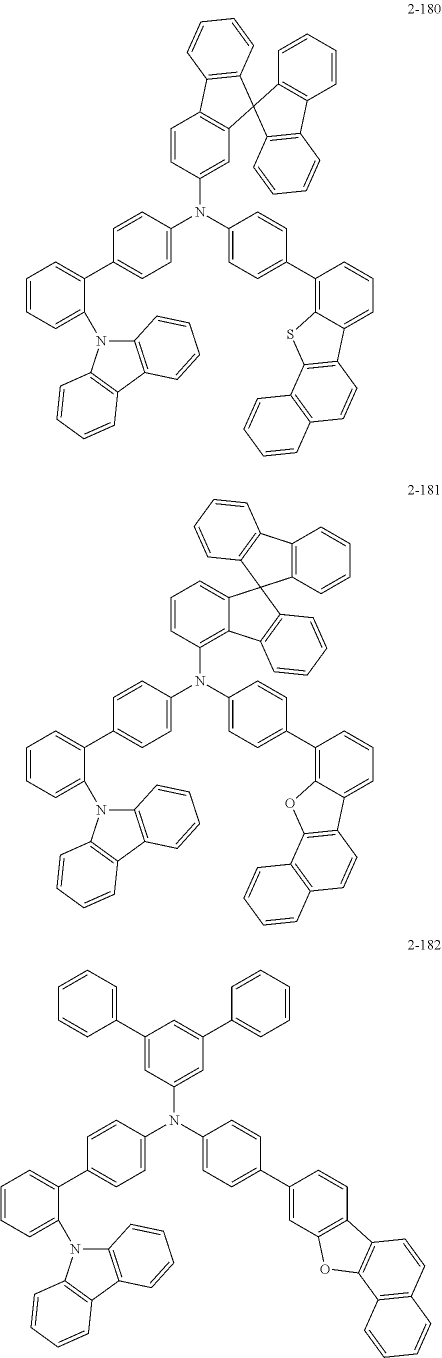

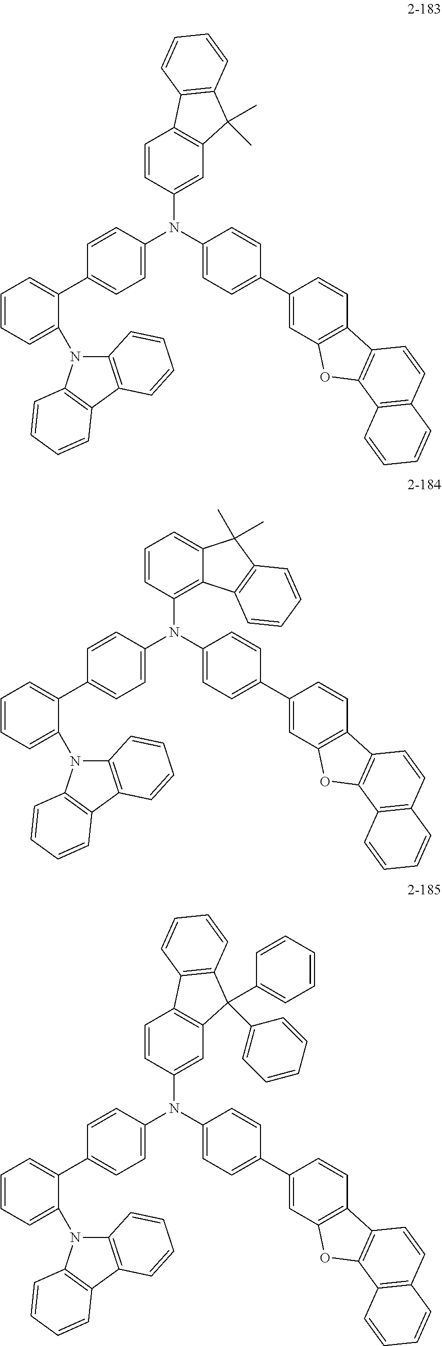 [SMILES: CC1(C)c2ccccc2-c2ccc(N(c3ccc(-c4ccc5c(c4)oc4c6ccccc6ccc54)cc3)c3ccc(-c4ccccc4-n4c5ccccc5c5ccccc54)cc3)cc21 ; CC1(C)c2ccccc2-c2c(N(c3ccc(-c4ccc5c(c4)oc4c6ccccc6ccc54)cc3)c3ccc(-c4ccccc4-n4c5ccccc5c5ccccc54)cc3)cccc21 ; c1ccc(C2(c3ccccc3)c3ccccc3-c3ccc(N(c4ccc(-c5ccc6c(c5)oc5c7ccccc7ccc65)cc4)c4ccc(-c5ccccc5-n5c6ccccc6c6ccccc65)cc4)cc32)cc1]

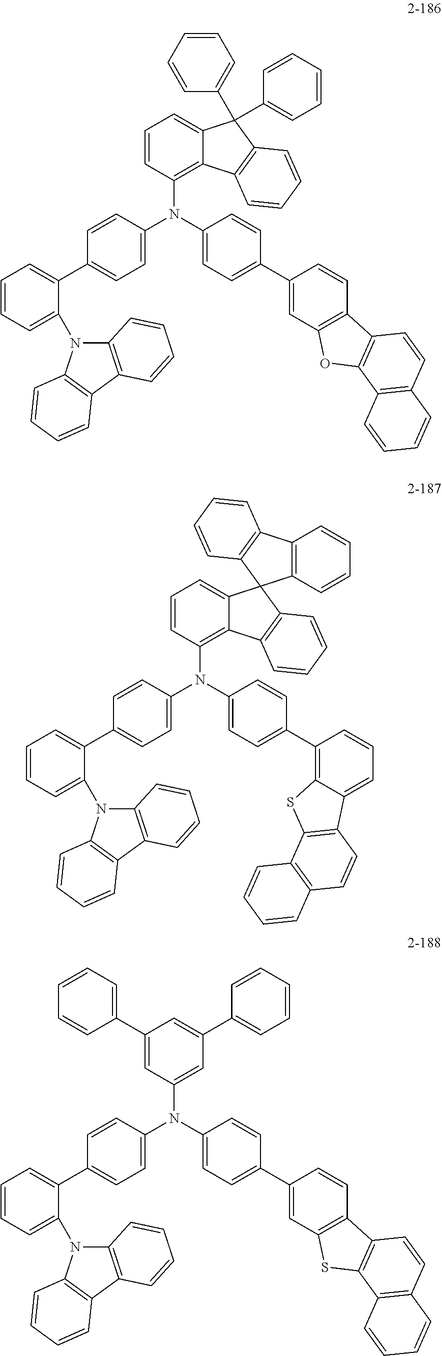

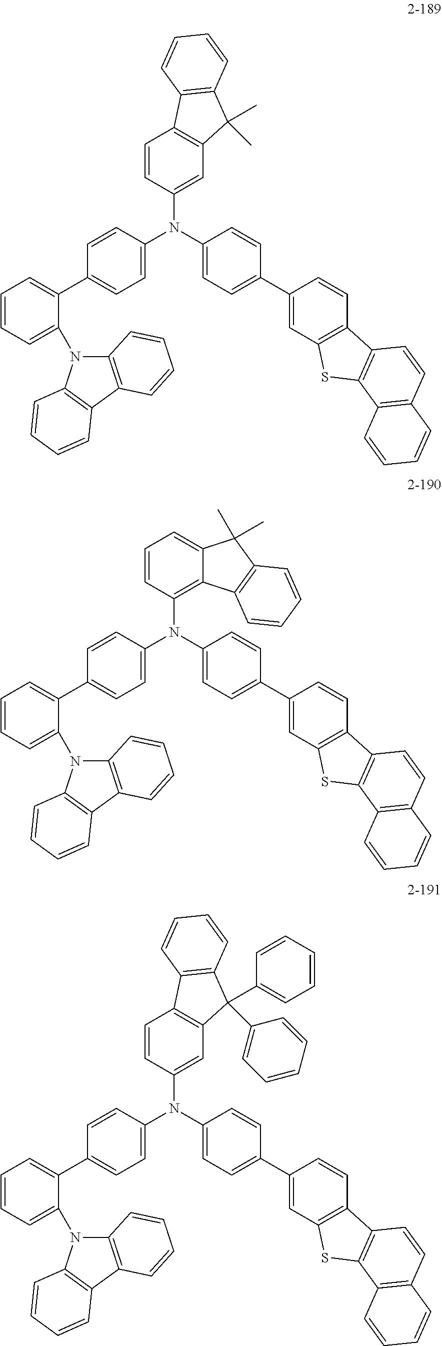 [SMILES: CC1(C)c2ccccc2-c2ccc(N(c3ccc(-c4ccc5c(c4)sc4c6ccccc6ccc54)cc3)c3ccc(-c4ccccc4-n4c5ccccc5c5ccccc54)cc3)cc21 ; CC1(C)c2ccccc2-c2c(N(c3ccc(-c4ccc5c(c4)sc4c6ccccc6ccc54)cc3)c3ccc(-c4ccccc4-n4c5ccccc5c5ccccc54)cc3)cccc21 ; c1ccc(C2(c3ccccc3)c3ccccc3-c3ccc(N(c4ccc(-c5ccc6c(c5)sc5c7ccccc7ccc65)cc4)c4ccc(-c5ccccc5-n5c6ccccc6c6ccccc65)cc4)cc32)cc1]

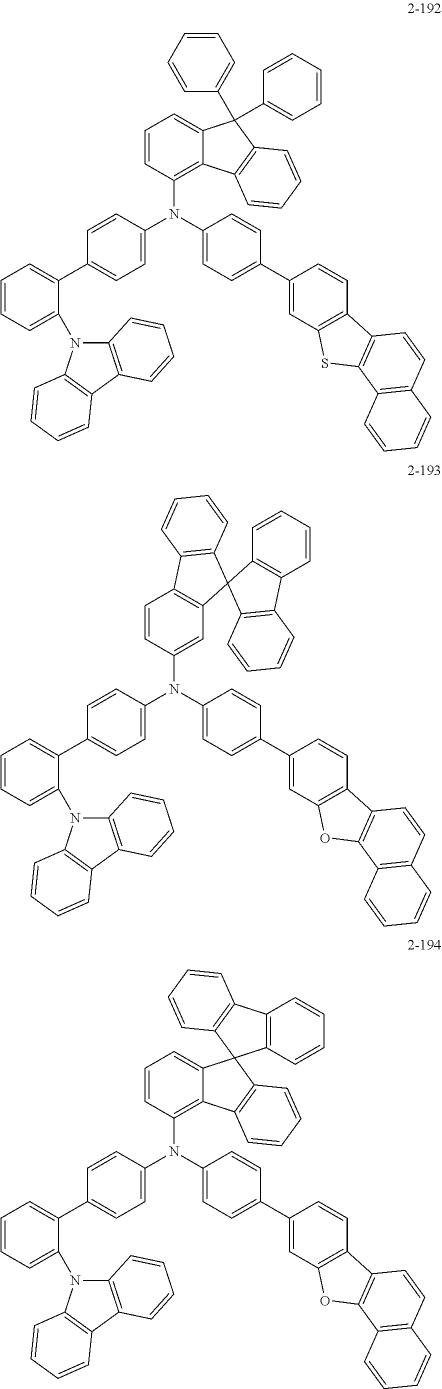 [SMILES: c1ccc(C2(c3ccccc3)c3ccccc3-c3c(N(c4ccc(-c5ccc6c(c5)sc5c7ccccc7ccc65)cc4)c4ccc(-c5ccccc5-n5c6ccccc6c6ccccc65)cc4)cccc32)cc1 ; c1ccc(-n2c3ccccc3c3ccccc32)c(-c2ccc(N(c3ccc(-c4ccc5c(c4)oc4c6ccccc6ccc54)cc3)c3ccc4c(c3)C3(c5ccccc5-c5ccccc53)c3ccccc3-4)cc2)c1 ; c1ccc(-n2c3ccccc3c3ccccc32)c(-c2ccc(N(c3ccc(-c4ccc5c(c4)oc4c6ccccc6ccc54)cc3)c3cccc4c3-c3ccccc3C43c4ccccc4-c4ccccc43)cc2)c1]

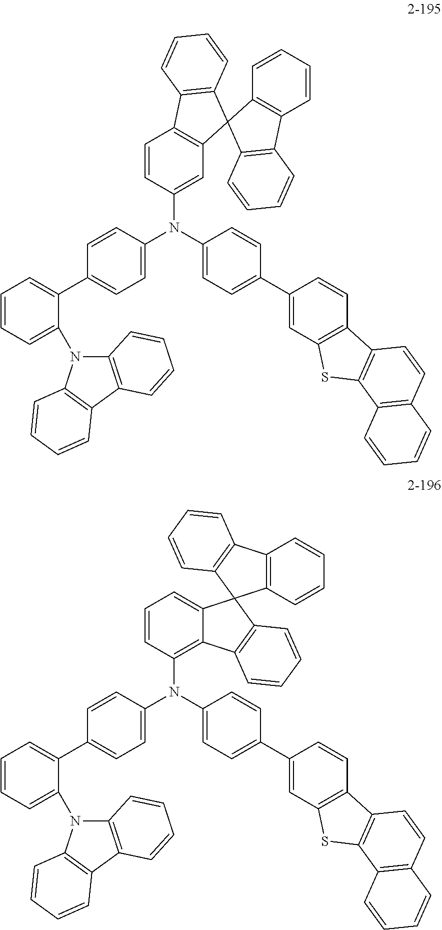 [SMILES: c1ccc(-n2c3ccccc3c3ccccc32)c(-c2ccc(N(c3ccc(-c4ccc5c(c4)sc4c6ccccc6ccc54)cc3)c3ccc4c(c3)C3(c5ccccc5-c5ccccc53)c3ccccc3-4)cc2)c1 ; c1ccc(-n2c3ccccc3c3ccccc32)c(-c2ccc(N(c3ccc(-c4ccc5c(c4)sc4c6ccccc6ccc54)cc3)c3cccc4c3-c3ccccc3C43c4ccccc4-c4ccccc43)cc2)c1]

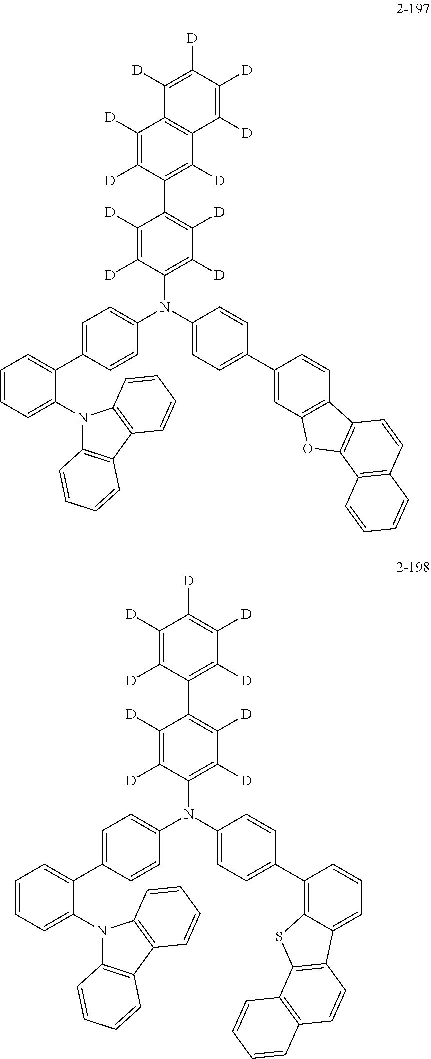 [SMILES: [2H]c1c([2H])c(N(c2ccc(-c3ccc4c(c3)oc3c5ccccc5ccc43)cc2)c2ccc(-c3ccccc3-n3c4ccccc4c4ccccc43)cc2)c([2H])c([2H])c1-c1c([2H])c([2H])c2c([2H])c([2H])c([2H])c([2H])c2c1[2H] ; [2H]c1c([2H])c([2H])c(-c2c([2H])c([2H])c(N(c3ccc(-c4ccccc4-n4c5ccccc5c5ccccc54)cc3)c3ccc(-c4cccc5c4sc4c6ccccc6ccc54)cc3)c([2H])c2[2H])c([2H])c1[2H]]

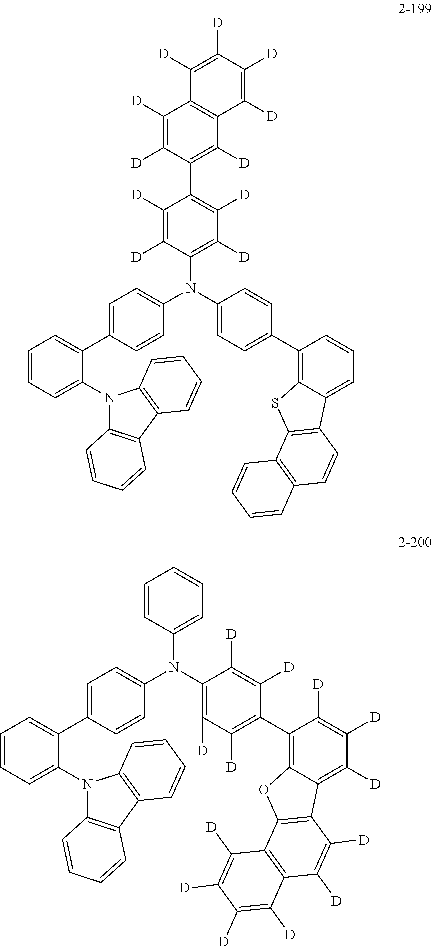 [SMILES: [2H]c1c([2H])c(N(c2ccc(-c3ccccc3-n3c4ccccc4c4ccccc43)cc2)c2ccc(-c3cccc4c3sc3c5ccccc5ccc43)cc2)c([2H])c([2H])c1-c1c([2H])c([2H])c2c([2H])c([2H])c([2H])c([2H])c2c1[2H] ; [2H]c1c([2H])c(N(c2ccccc2)c2ccc(-c3ccccc3-n3c4ccccc4c4ccccc43)cc2)c([2H])c([2H])c1-c1c([2H])c([2H])c([2H])c2c1oc1c3c([2H])c([2H])c([2H])c([2H])c3c([2H])c([2H])c12]

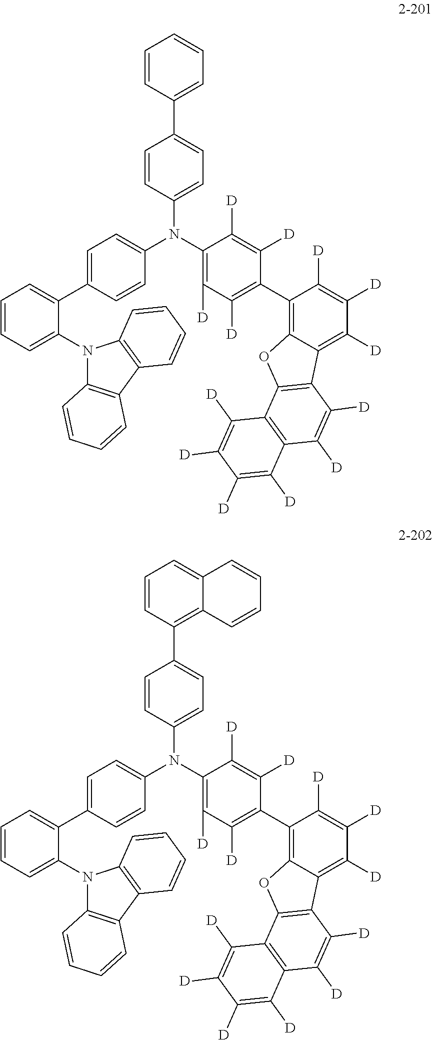 [SMILES: [2H]c1c([2H])c(N(c2ccc(-c3ccccc3)cc2)c2ccc(-c3ccccc3-n3c4ccccc4c4ccccc43)cc2)c([2H])c([2H])c1-c1c([2H])c([2H])c([2H])c2c1oc1c3c([2H])c([2H])c([2H])c([2H])c3c([2H])c([2H])c12 ; [2H]c1c([2H])c(N(c2ccc(-c3ccccc3-n3c4ccccc4c4ccccc43)cc2)c2ccc(-c3cccc4ccccc34)cc2)c([2H])c([2H])c1-c1c([2H])c([2H])c([2H])c2c1oc1c3c([2H])c([2H])c([2H])c([2H])c3c([2H])c([2H])c12]

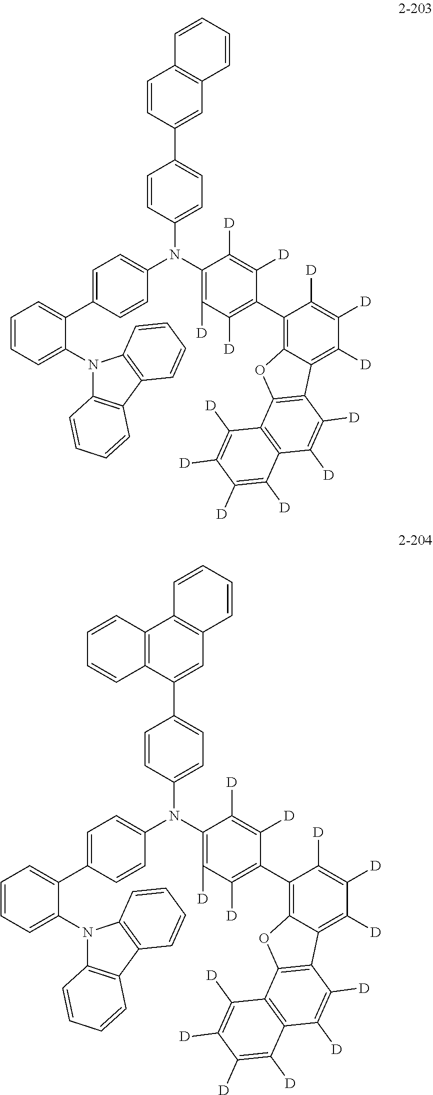 [SMILES: [2H]c1c([2H])c(N(c2ccc(-c3ccc4ccccc4c3)cc2)c2ccc(-c3ccccc3-n3c4ccccc4c4ccccc43)cc2)c([2H])c([2H])c1-c1c([2H])c([2H])c([2H])c2c1oc1c3c([2H])c([2H])c([2H])c([2H])c3c([2H])c([2H])c12 ; [2H]c1c([2H])c(N(c2ccc(-c3ccccc3-n3c4ccccc4c4ccccc43)cc2)c2ccc(-c3cc4ccccc4c4ccccc34)cc2)c([2H])c([2H])c1-c1c([2H])c([2H])c([2H])c2c1oc1c3c([2H])c([2H])c([2H])c([2H])c3c([2H])c([2H])c12]

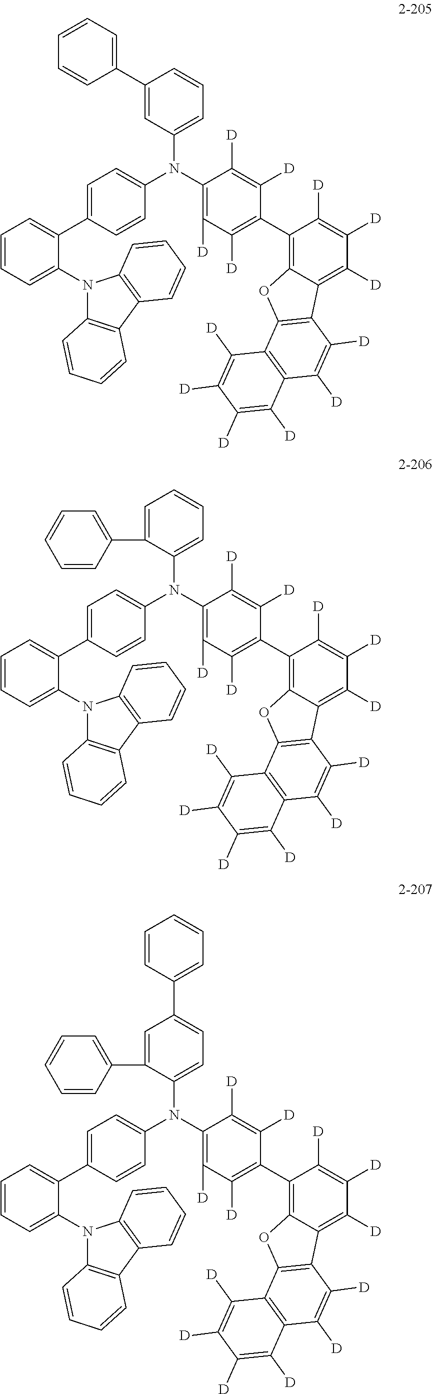

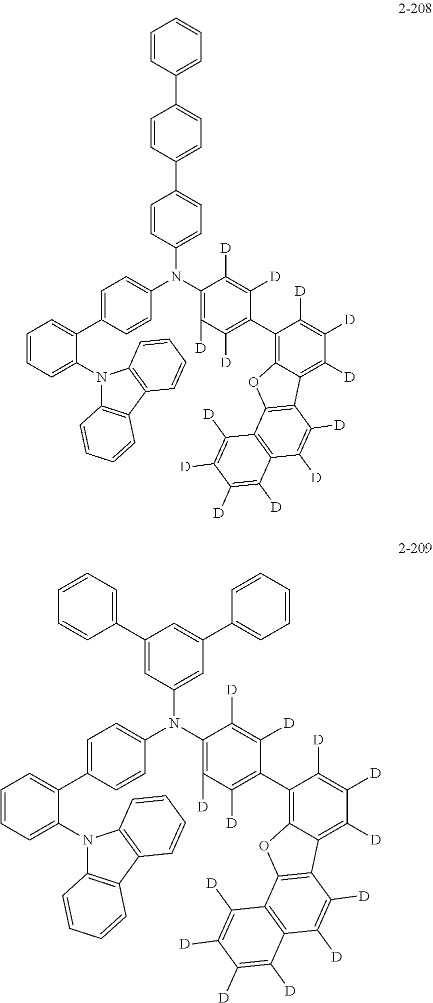 [SMILES: [2H]c1c([2H])c(N(c2ccc(-c3ccc(-c4ccccc4)cc3)cc2)c2ccc(-c3ccccc3-n3c4ccccc4c4ccccc43)cc2)c([2H])c([2H])c1-c1c([2H])c([2H])c([2H])c2c1oc1c3c([2H])c([2H])c([2H])c([2H])c3c([2H])c([2H])c12 ; [2H]c1c([2H])c(N(c2ccc(-c3ccccc3-n3c4ccccc4c4ccccc43)cc2)c2cc(-c3ccccc3)cc(-c3ccccc3)c2)c([2H])c([2H])c1-c1c([2H])c([2H])c([2H])c2c1oc1c3c([2H])c([2H])c([2H])c([2H])c3c([2H])c([2H])c12]

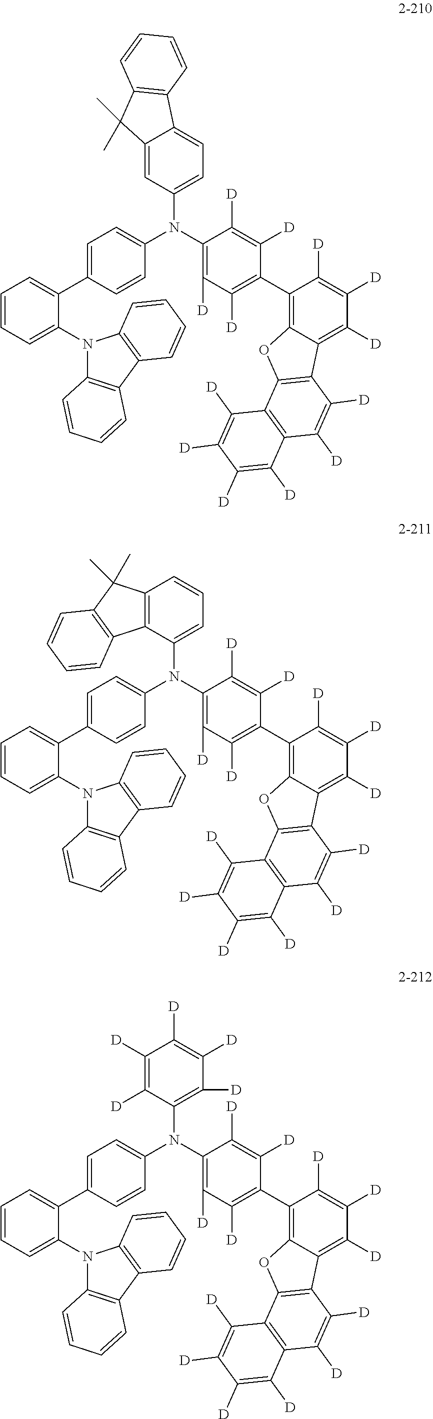

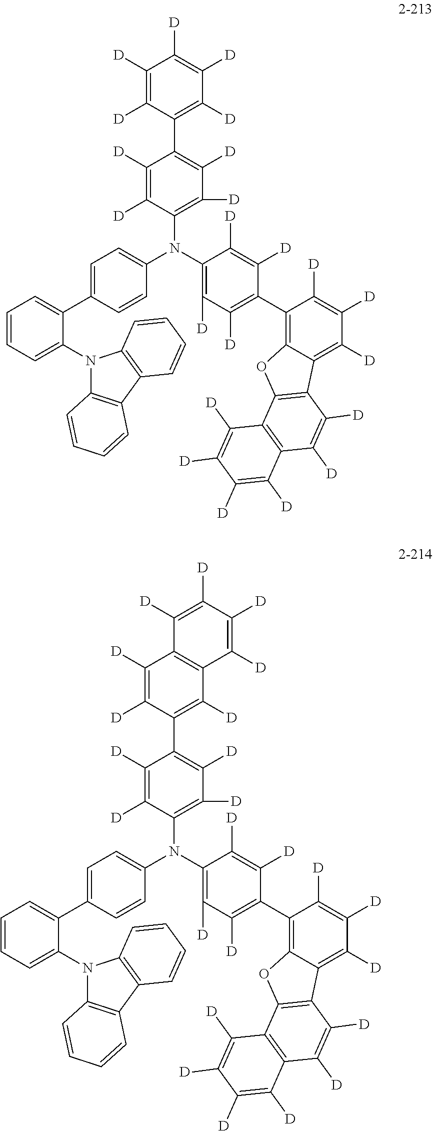 [SMILES: [2H]c1c([2H])c([2H])c(-c2c([2H])c([2H])c(N(c3ccc(-c4ccccc4-n4c5ccccc5c5ccccc54)cc3)c3c([2H])c([2H])c(-c4c([2H])c([2H])c([2H])c5c4oc4c6c([2H])c([2H])c([2H])c([2H])c6c([2H])c([2H])c45)c([2H])c3[2H])c([2H])c2[2H])c([2H])c1[2H] ; [2H]c1c([2H])c(N(c2ccc(-c3ccccc3-n3c4ccccc4c4ccccc43)cc2)c2c([2H])c([2H])c(-c3c([2H])c([2H])c([2H])c4c3oc3c5c([2H])c([2H])c([2H])c([2H])c5c([2H])c([2H])c34)c([2H])c2[2H])c([2H])c([2H])c1-c1c([2H])c([2H])c2c([2H])c([2H])c([2H])c([2H])c2c1[2H]]

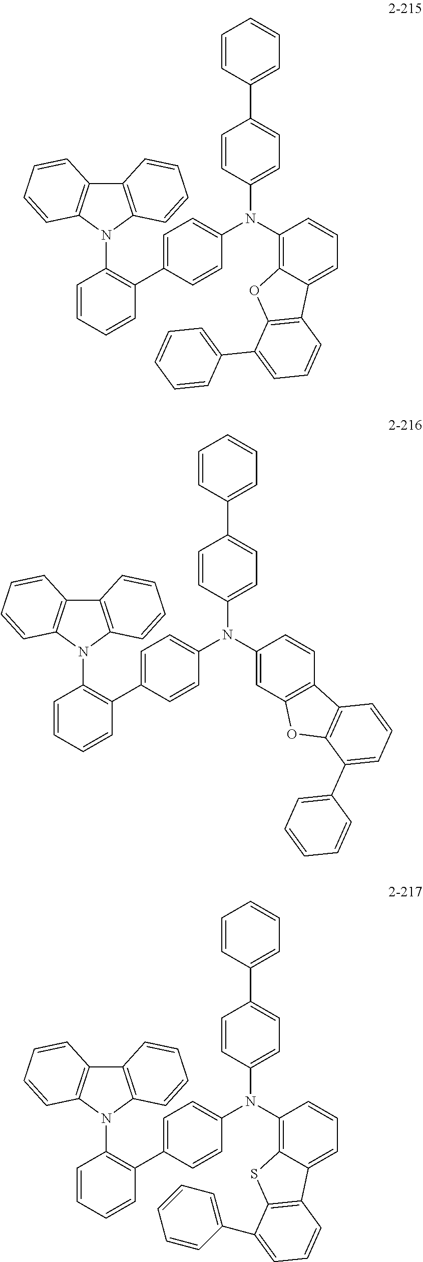 [SMILES: c1ccc(-c2ccc(N(c3ccc(-c4ccccc4-n4c5ccccc5c5ccccc54)cc3)c3cccc4c3oc3c(-c5ccccc5)cccc34)cc2)cc1 ; c1ccc(-c2ccc(N(c3ccc(-c4ccccc4-n4c5ccccc5c5ccccc54)cc3)c3ccc4c(c3)oc3c(-c5ccccc5)cccc34)cc2)cc1 ; c1ccc(-c2ccc(N(c3ccc(-c4ccccc4-n4c5ccccc5c5ccccc54)cc3)c3cccc4c3sc3c(-c5ccccc5)cccc34)cc2)cc1]

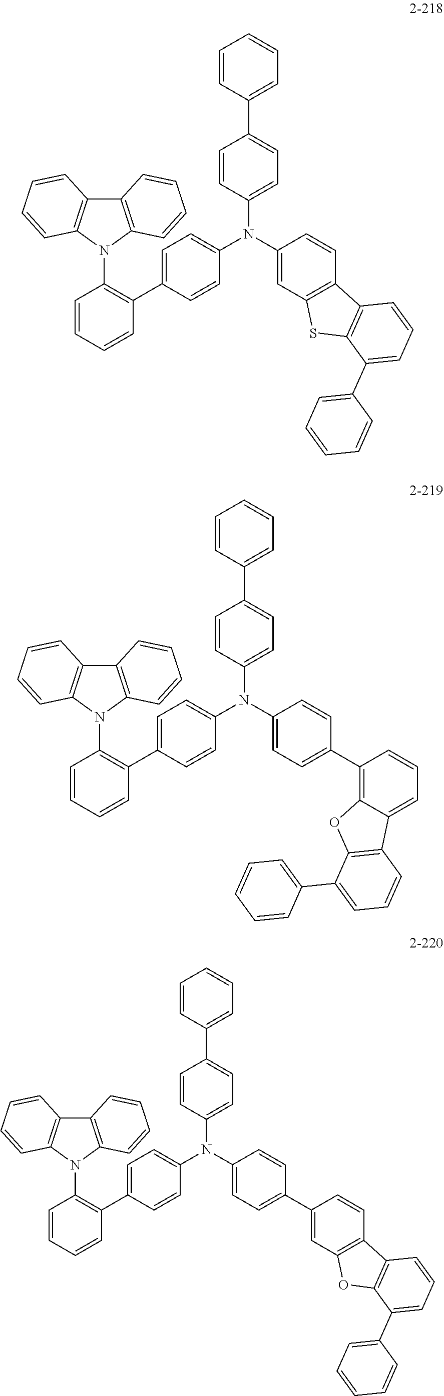 [SMILES: c1ccc(-c2ccc(N(c3ccc(-c4ccccc4-n4c5ccccc5c5ccccc54)cc3)c3ccc4c(c3)sc3c(-c5ccccc5)cccc34)cc2)cc1 ; c1ccc(-c2ccc(N(c3ccc(-c4ccccc4-n4c5ccccc5c5ccccc54)cc3)c3ccc(-c4cccc5c4oc4c(-c6ccccc6)cccc45)cc3)cc2)cc1 ; c1ccc(-c2ccc(N(c3ccc(-c4ccc5c(c4)oc4c(-c6ccccc6)cccc45)cc3)c3ccc(-c4ccccc4-n4c5ccccc5c5ccccc54)cc3)cc2)cc1]

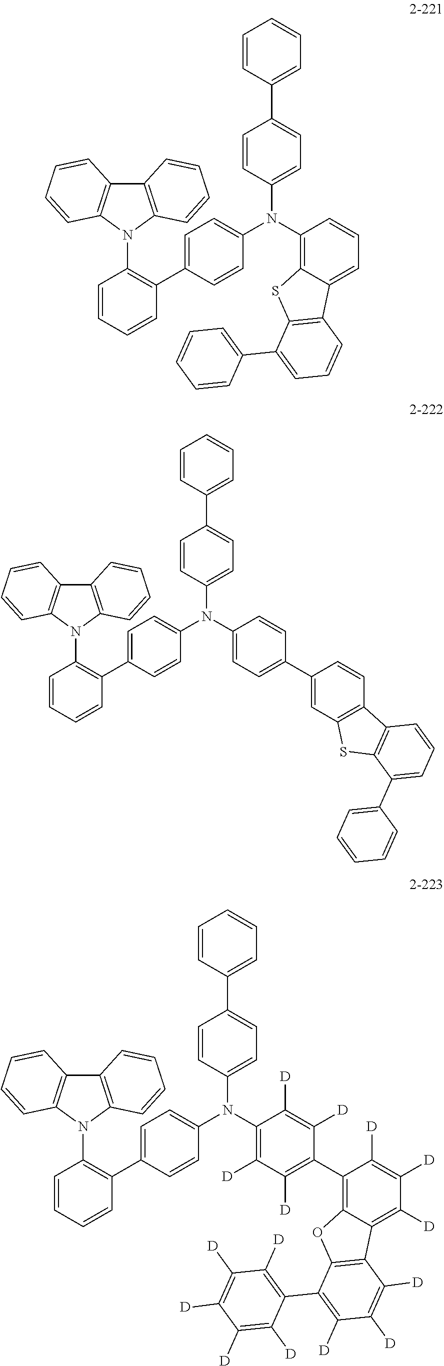 [SMILES: c1ccc(-c2ccc(N(c3ccc(-c4ccccc4-n4c5ccccc5c5ccccc54)cc3)c3cccc4c3sc3c(-c5ccccc5)cccc34)cc2)cc1 ; c1ccc(-c2ccc(N(c3ccc(-c4ccc5c(c4)sc4c(-c6ccccc6)cccc45)cc3)c3ccc(-c4ccccc4-n4c5ccccc5c5ccccc54)cc3)cc2)cc1 ; [2H]c1c([2H])c([2H])c(-c2c([2H])c([2H])c([2H])c3c2oc2c(-c4c([2H])c([2H])c(N(c5ccc(-c6ccccc6)cc5)c5ccc(-c6ccccc6-n6c7ccccc7c7ccccc76)cc5)c([2H])c4[2H])c([2H])c([2H])c([2H])c23)c([2H])c1[2H]]

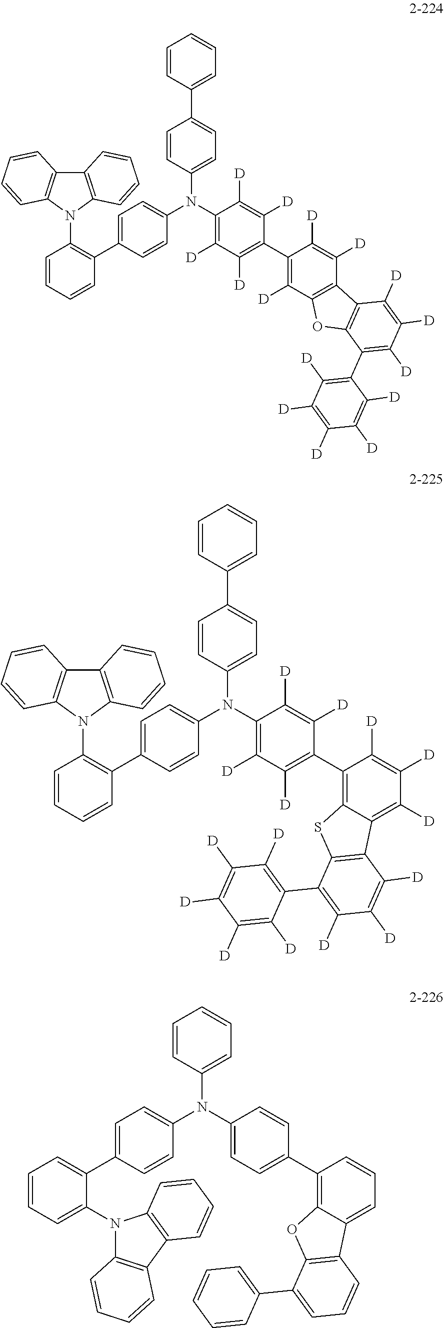 [SMILES: [2H]c1c([2H])c([2H])c(-c2c([2H])c([2H])c([2H])c3c2oc2c([2H])c(-c4c([2H])c([2H])c(N(c5ccc(-c6ccccc6)cc5)c5ccc(-c6ccccc6-n6c7ccccc7c7ccccc76)cc5)c([2H])c4[2H])c([2H])c([2H])c23)c([2H])c1[2H] ; [2H]c1c([2H])c([2H])c(-c2c([2H])c([2H])c([2H])c3c2sc2c(-c4c([2H])c([2H])c(N(c5ccc(-c6ccccc6)cc5)c5ccc(-c6ccccc6-n6c7ccccc7c7ccccc76)cc5)c([2H])c4[2H])c([2H])c([2H])c([2H])c23)c([2H])c1[2H] ; c1ccc(-c2cccc3c2oc2c(-c4ccc(N(c5ccccc5)c5ccc(-c6ccccc6-n6c7ccccc7c7ccccc76)cc5)cc4)cccc23)cc1]

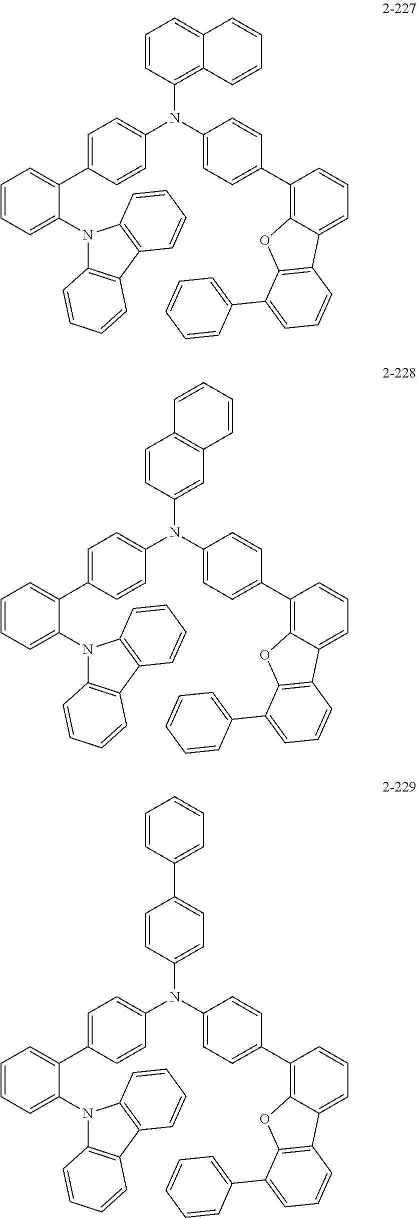

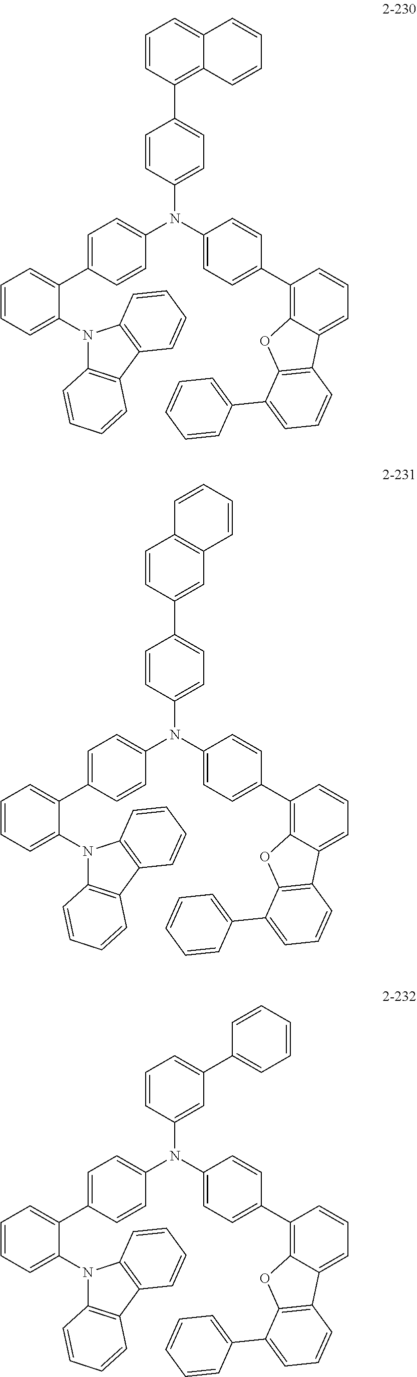 [SMILES: c1ccc(-c2cccc3c2oc2c(-c4ccc(N(c5ccc(-c6ccccc6-n6c7ccccc7c7ccccc76)cc5)c5ccc(-c6cccc7ccccc67)cc5)cc4)cccc23)cc1 ; c1ccc(-c2cccc3c2oc2c(-c4ccc(N(c5ccc(-c6ccc7ccccc7c6)cc5)c5ccc(-c6ccccc6-n6c7ccccc7c7ccccc76)cc5)cc4)cccc23)cc1 ; c1ccc(-c2cccc(N(c3ccc(-c4ccccc4-n4c5ccccc5c5ccccc54)cc3)c3ccc(-c4cccc5c4oc4c(-c6ccccc6)cccc45)cc3)c2)cc1]

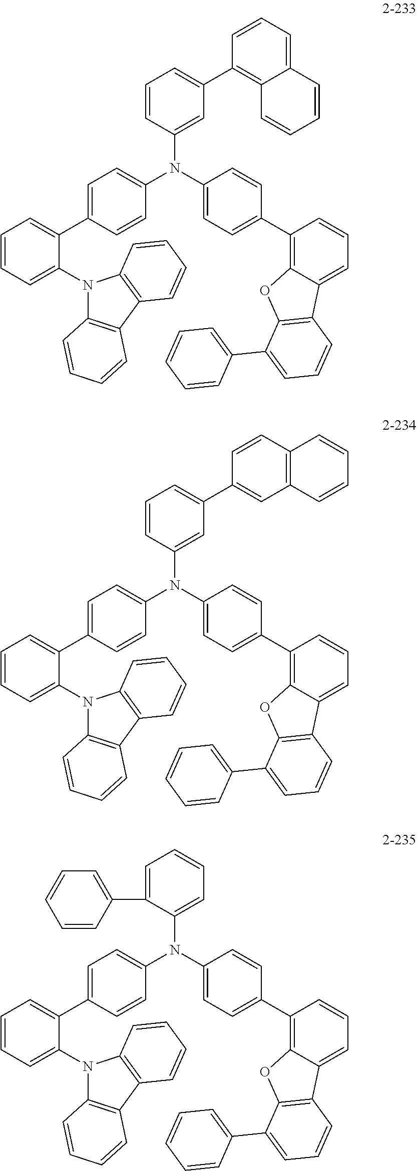

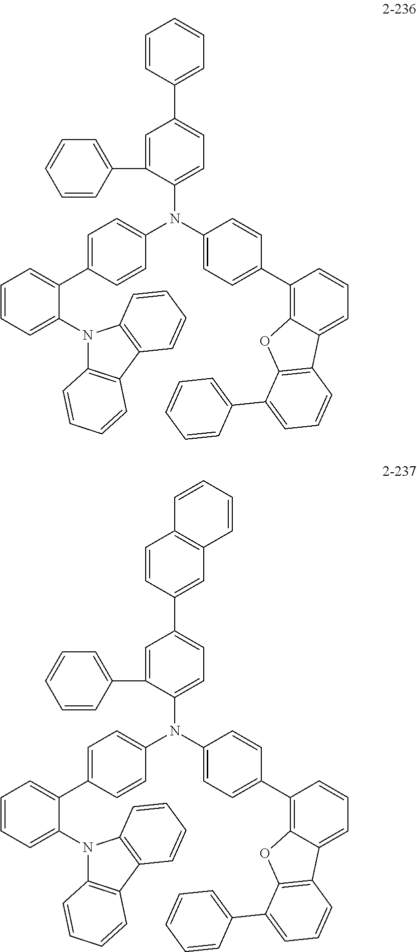

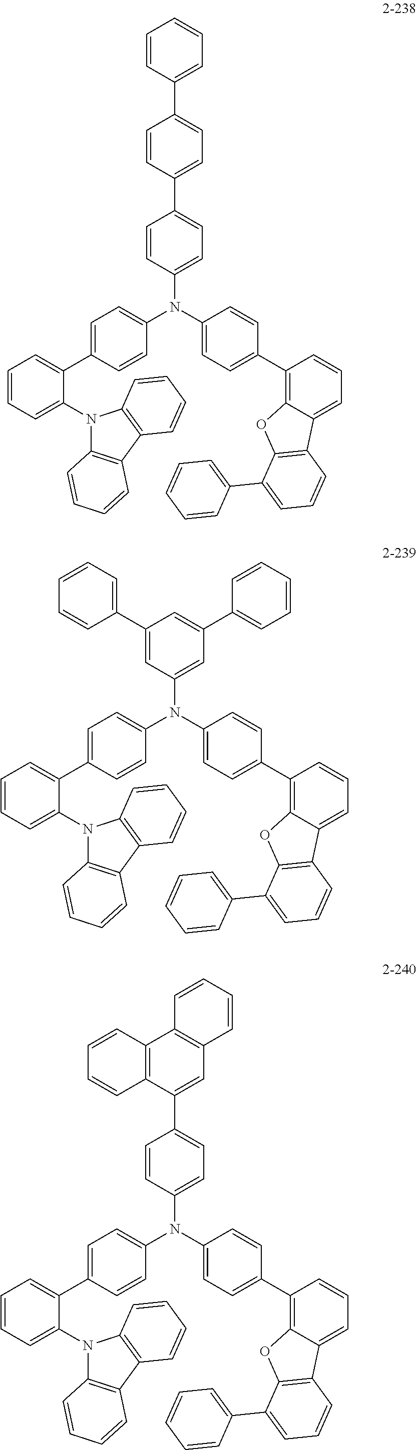 [SMILES: c1ccc(-c2ccc(-c3ccc(N(c4ccc(-c5ccccc5-n5c6ccccc6c6ccccc65)cc4)c4ccc(-c5cccc6c5oc5c(-c7ccccc7)cccc56)cc4)cc3)cc2)cc1 ; c1ccc(-c2cc(-c3ccccc3)cc(N(c3ccc(-c4ccccc4-n4c5ccccc5c5ccccc54)cc3)c3ccc(-c4cccc5c4oc4c(-c6ccccc6)cccc45)cc3)c2)cc1 ; c1ccc(-c2cccc3c2oc2c(-c4ccc(N(c5ccc(-c6ccccc6-n6c7ccccc7c7ccccc76)cc5)c5ccc(-c6cc7ccccc7c7ccccc67)cc5)cc4)cccc23)cc1]

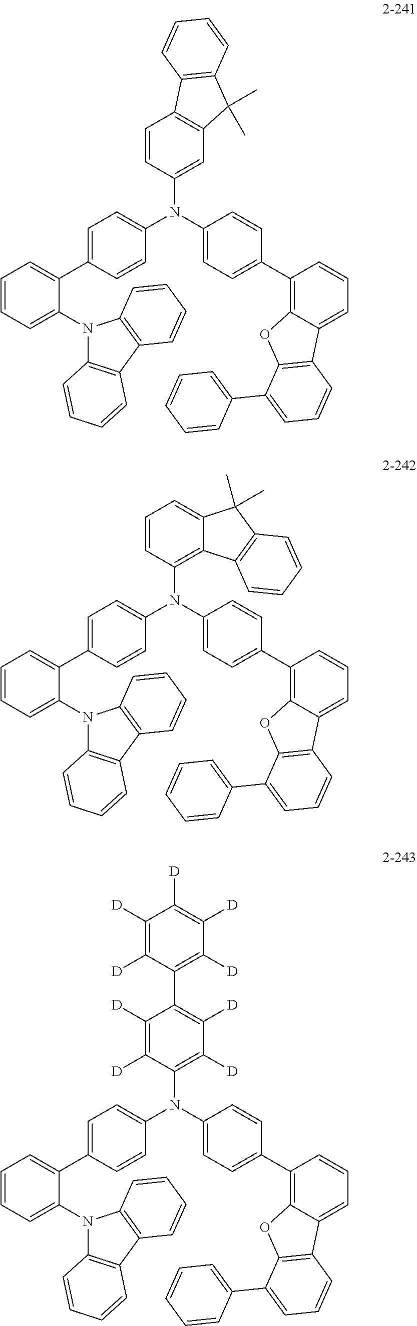

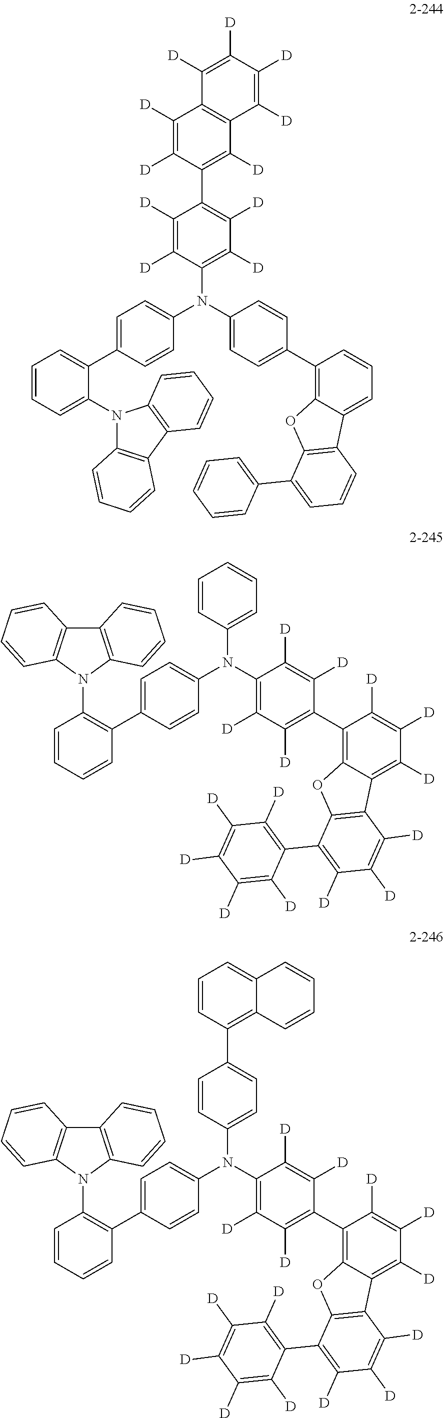

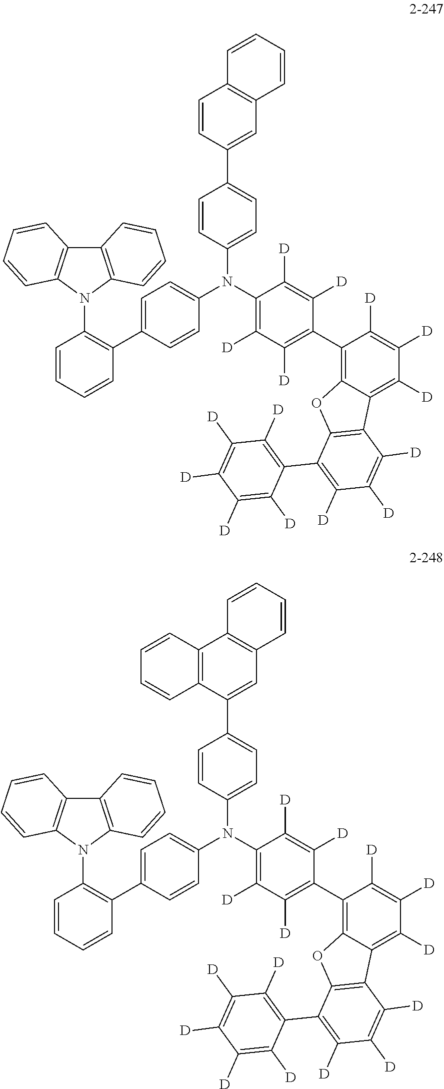 [SMILES: [2H]c1c([2H])c([2H])c(-c2c([2H])c([2H])c([2H])c3c2oc2c(-c4c([2H])c([2H])c(N(c5ccc(-c6ccc7ccccc7c6)cc5)c5ccc(-c6ccccc6-n6c7ccccc7c7ccccc76)cc5)c([2H])c4[2H])c([2H])c([2H])c([2H])c23)c([2H])c1[2H] ; [2H]c1c([2H])c([2H])c(-c2c([2H])c([2H])c([2H])c3c2oc2c(-c4c([2H])c([2H])c(N(c5ccc(-c6ccccc6-n6c7ccccc7c7ccccc76)cc5)c5ccc(-c6cc7ccccc7c7ccccc67)cc5)c([2H])c4[2H])c([2H])c([2H])c([2H])c23)c([2H])c1[2H]]

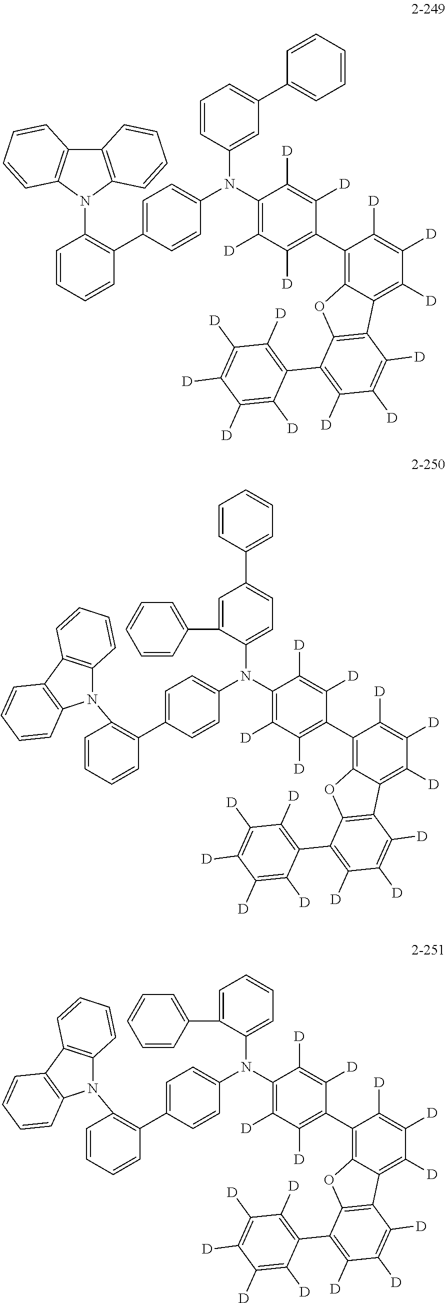

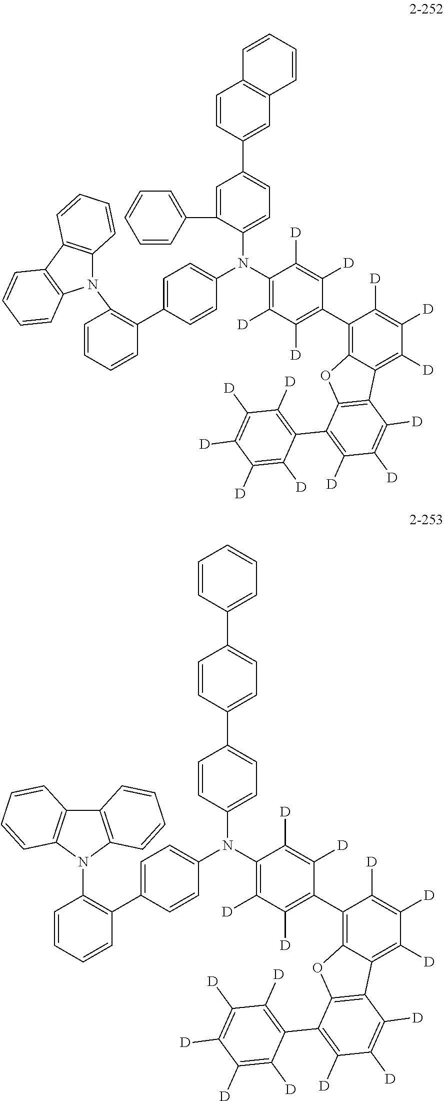 [SMILES: [2H]c1c([2H])c([2H])c(-c2c([2H])c([2H])c([2H])c3c2oc2c(-c4c([2H])c([2H])c(N(c5ccc(-c6ccccc6-n6c7ccccc7c7ccccc76)cc5)c5ccc(-c6ccc7ccccc7c6)cc5-c5ccccc5)c([2H])c4[2H])c([2H])c([2H])c([2H])c23)c([2H])c1[2H] ; [2H]c1c([2H])c([2H])c(-c2c([2H])c([2H])c([2H])c3c2oc2c(-c4c([2H])c([2H])c(N(c5ccc(-c6ccc(-c7ccccc7)cc6)cc5)c5ccc(-c6ccccc6-n6c7ccccc7c7ccccc76)cc5)c([2H])c4[2H])c([2H])c([2H])c([2H])c23)c([2H])c1[2H]]

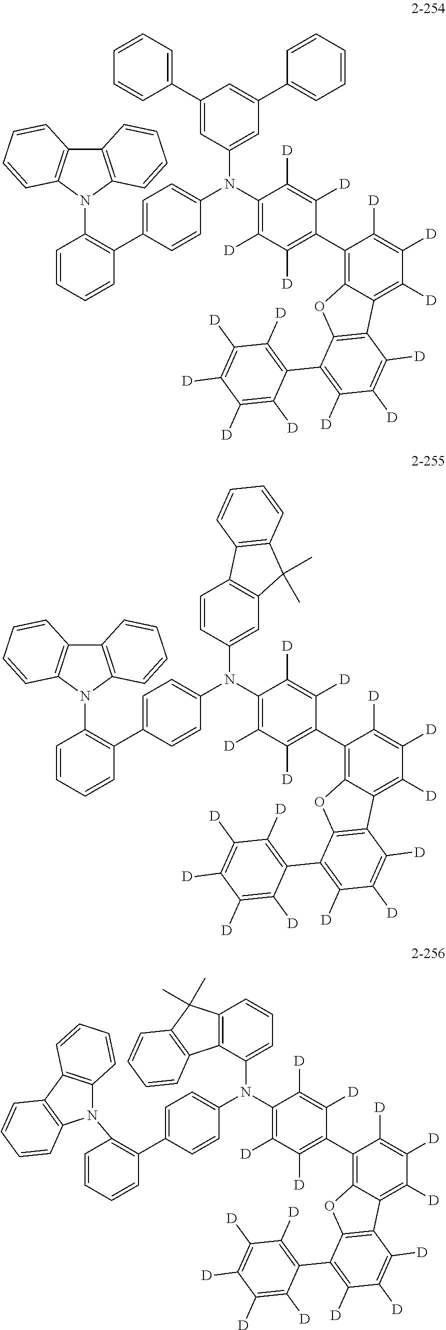

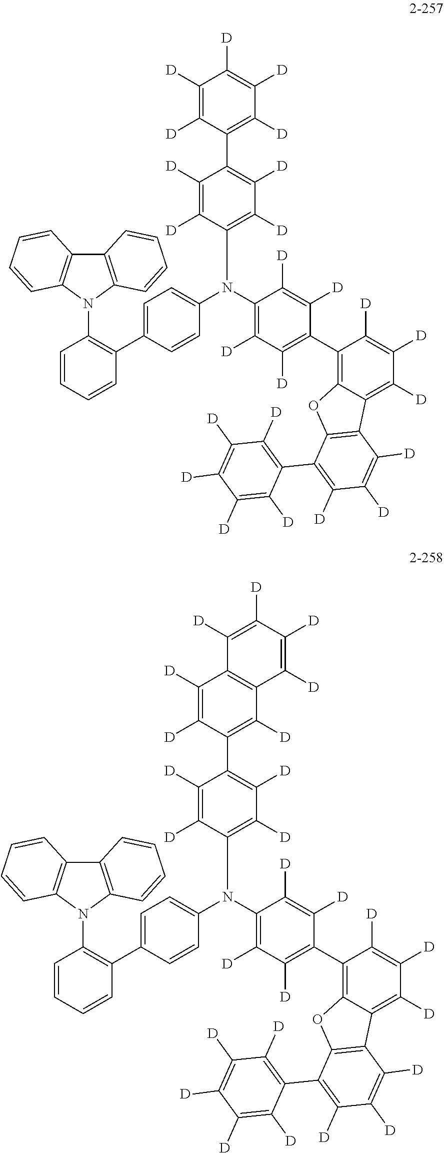 [SMILES: [2H]c1c([2H])c([2H])c(-c2c([2H])c([2H])c(N(c3ccc(-c4ccccc4-n4c5ccccc5c5ccccc54)cc3)c3c([2H])c([2H])c(-c4c([2H])c([2H])c([2H])c5c4oc4c(-c6c([2H])c([2H])c([2H])c([2H])c6[2H])c([2H])c([2H])c([2H])c45)c([2H])c3[2H])c([2H])c2[2H])c([2H])c1[2H] ; [2H]c1c([2H])c([2H])c(-c2c([2H])c([2H])c([2H])c3c2oc2c(-c4c([2H])c([2H])c(N(c5ccc(-c6ccccc6-n6c7ccccc7c7ccccc76)cc5)c5c([2H])c([2H])c(-c6c([2H])c([2H])c7c([2H])c([2H])c([2H])c([2H])c7c6[2H])c([2H])c5[2H])c([2H])c4[2H])c([2H])c([2H])c([2H])c23)c([2H])c1[2H]]

The compound of Chemical Formula 1 of the present invention can be usefully used as a material for a hole transport auxiliary layer.

When the organic compound is used as the material for an organic electroluminescent device, the organic material can show equivalent or excellent characteristics in most device characteristics, such as luminous efficiency and lifetime.

The present invention provides an organic electroluminescent device containing the compound represented by Chemical Formula 1.

The organic compound of the present invention can be usefully used as a material for a hole transport auxiliary layer.

In addition, according to the present invention, in an organic electroluminescent device in which an organic thin film layer including one or more layers including at least a light emitting layer is stacked between a positive electrode and a negative electrode, the organic thin film layer is a hole transport layer and/or a hole transport auxiliary layer between the first electrode and the light emitting layer.

The hole transport layer and/or the hole transport auxiliary layer is the compound represented by Chemical Formula 1.

The hole transport auxiliary layer may adjust hole injection characteristics by reducing a difference in HOMO energy level between the hole transport layer and the light emitting layer, thereby reducing the accumulation of holes at an interface between the hole transport auxiliary layer and the light emitting layer to reduce a quenching phenomenon in which excitons extinct due to polarons at the interface. Therefore, it is possible to reduce a degradation phenomenon of the device, thereby stabilizing the device and increasing efficiency and lifetime.

The organic electroluminescent device may have a structure in which a positive electrode, a hole injection layer, a hole transport layer, a hole transport auxiliary layer, a light emitting layer, an electron transport layer, an electron injection layer, and a negative electrode are stacked, and an electron transport auxiliary layer may be added if necessary.

Hereinafter, the organic electroluminescent device of the present invention will be described as an example. However, the contents illustrated below do not limit the organic electroluminescent device of the present invention.

The organic electroluminescent device of the present invention may have a structure in which a positive electrode (hole injection electrode), a hole injection layer (HIL), a hole transport layer (HTL), a hole transport auxiliary layer, a light emitting layer (EML), and a negative electrode (electron injection electrode) are sequentially stacked and preferably, may further include a hole transport auxiliary layer between the positive electrode and the light emitting layer, and an electron transport layer (ETL) and an electron injection layer (EIL) between the negative electrode and the light emitting layer. In addition, an electron transport auxiliary layer may be further included between the negative electrode and the light emitting layer.

In a method of manufacturing an organic electroluminescent device according to the present invention, a positive electrode is formed by coating a surface of a substrate with a material for a positive electrode in a typical method. In this case, the substrate used is preferably a glass substrate or a transparent plastic substrate with excellent transparency, surface smoothness, ease of handling, and waterproofness. In addition, indium tin oxide (ITO), indium zinc oxide (IZO), tin oxide (SnO₂), zinc oxide (ZnO), and the like, which are transparent and have excellent conductivity, can be used as a material for a positive electrode.

Next, a hole injection layer is formed by forming a material for a hole injection layer (HIL) on a surface of the positive electrode by vacuum thermal evaporation or spin coating in a typical method. Examples of the material for a hole injection layer may include copper phthalocyanine (CuPc), 4,4′,4″-tris(3-methylphenylamino)triphenylamine (m-MTDATA), 4,4′,4″-tris(3-methylphenyl)phenoxybenzene (m-MTDAPB), 4,4′,4″-tri(N-carbazolyl)triphenylamine (TCTA) which is starburst type amines, 4,4′,4″-tris(N-(2-naphthyl)-N-phenylamino)-triphenylamine(2-TNATA), or IDE406 available from Idemitsu.

A hole transport layer is formed by forming a material for a hole transport layer on a surface of the hole injection layer by vacuum thermal evaporation or spin coating in a typical method. As the material for a hole transport layer, a commonly used material for a hole transport layer can be used.

A hole transport auxiliary layer may be formed by forming the compound represented by Chemical Formula 1 according to the present invention on a surface of the hole transport layer by vacuum thermal evaporation or spin coating. As described above, in the hole transport auxiliary layer, the compound according to the present invention can be used as the material for a hole transport auxiliary layer, and a commonly used material for a hole transport auxiliary layer can be used to form the hole transport auxiliary layer.

A light emitting layer is formed by forming a material for a light emitting layer (EML) on a surface of the hole transport auxiliary layer by vacuum thermal evaporation or spin coating in a typical method. In this case, a sole light emitting material or light emitting host material among materials for a light emitting layer used may use tris(8-hydroxyquinolinolato)aluminum (Alq₃) or the like for green and use Alq₃, CBP(4,4′-N,N′-dicabazole-biphenyl, 4,4′-N,N′-dicabazole-biphenyl), PVK(poly(n-vinylcabazole), poly(n-vinylcarbazole)), ADN(9, 10-di(naphthalene-2-yl)anthracene, 9,10-di(naphthalene-2-yl)anthracene), TCTA, TPBI(1,3,5-tris(N-phenylbenzimidazole-2-yl)benzene, 1,3,5-tris(N-phenylbenzimidazol-2-yl)benzene), TBADN(3-tert-butyl-9,10-di(naphth-2-yl)anthracene, 3-tert-butyl-9,10-di(naphth-2-yl)anthracene, E3, DSA(distyrylarylene), or a mixture of two or more thereof for blue, but the present invention is not limited thereto.

In the case of dopant which can be used together with a light emitting host among the materials for a light emitting layer, IDE102 and IDE105 available from Idemitsu can be used, and as a phosphorescent dopant, tris(2-phenylpyridine)iridium(III)(Ir(ppy)3), iridium(III)bis[(4,6-difluorophenyl)pyridinato-N,C-2′]picolinate (FIrpic) (reference [Chihaya Adachi, et al., Appl. Phys. Lett., 2001, 79, 2082-2084]), platinium(II)octaethylporphyrin(PtOEP), TBE002 (by Covion), or the like can be used.

An electron transport layer is formed by forming a material for the electron transport layer (ETL) on a surface of the light emitting layer by vacuum thermal evaporation or spin coating in a typical method. In this case, the material for an electron transport layer used is not especially limited, and tris(8-hydroxyquinolinolato)aluminum(Alq₃) can be preferably used.

Optionally, by additionally forming a hole blocking layer (HBL) between the light emitting layer and the electron transport layer and using the phosphorescent dopant in the light emitting layer, it is possible to prevent triplet excitons or holes from diffusing into the electron transport layer.

The formation of the hole blocking layer may be performed by vacuum thermal evaporation and spin coating of a material for a hole blocking layer in a typical method, and the material for a hole blocking layer is not especially limited, but can preferably use (8-hydroxyquinolinolato)Lithium(Liq), bis(8-hydroxy-2-methylquinolinolnato)-aluminum biphenoxide(BAlq), bathocuproine(BCP), LiF, or the like.

An electron injection layer is formed by forming a material for an electron injection layer (EIL) on a surface of the electron transport layer by vacuum thermal evaporation or spin coating in a typical method. In this case, materials such as LiF, Liq, Li₂O, BaO, NaCl, and CsF can be used as the material for an electron injection layer.

A negative electrode is formed by forming a negative electrode material on a surface of the electron injection layer by vacuum thermal deposition in a typical method.

In this case, as the negative electrode material used, lithium (Li), aluminum (Al), aluminum-lithium (Al—Li), calcium (Ca), magnesium (Mg), magnesium-indium (Mg—In), and magnesium-silver (Mg—Ag), and the like can be used. In addition, in the case of a top-emitting organic electroluminescent device, a transparent negative electrode through which light may transmit may be formed by using indium tin oxide (ITO) or indium zinc oxide (IZO).

A capping layer CPL may be formed on a surface of the negative electrode using a composition for the formation of the capping layer.

Hereinafter, a method of synthesizing the compounds will be described below as a representative example. However, the method of synthesizing the compounds of the present invention is not limited to the method exemplified below, and the compounds of the present invention may be manufactured by the methods exemplified below and methods known in the art.

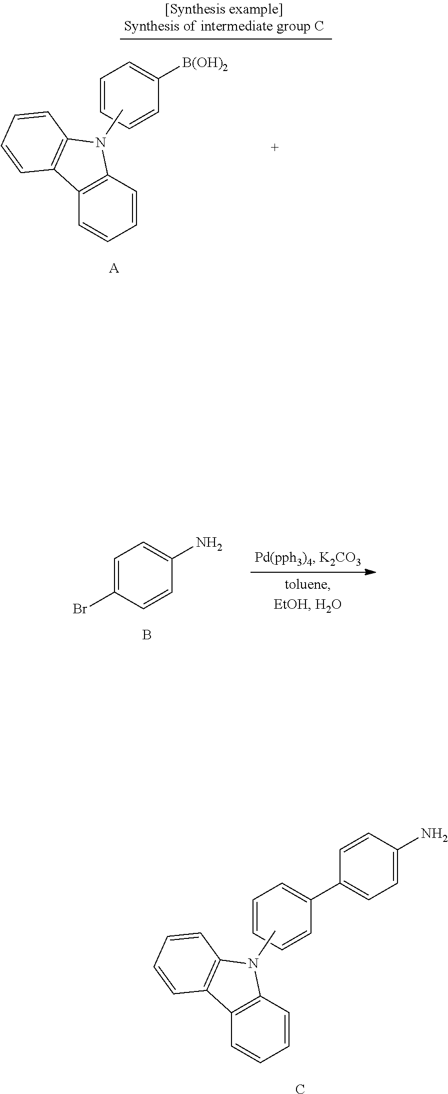

Reactant A (28.7 g, 100 mmol), reactant B (18.9 g, 110 mmol), Pd(PPh₃)₄ (2.31 g, 2.0 mmol), K₂CO₃ (27.6 g, 200 mmol) and toluene (150 mL), water (80 mL), and ethanol (50 mL) were added to a round bottom flask under nitrogen atmosphere and then stirred at 100° C. to perform reaction. When the reaction was finished, after separating an organic layer, then removing water, and performing filtering, the mixture was purified in a silica gel column and recrystallization method to manufacture intermediate group C.

The result is expressed in Table 1 below.

TABLE 1

| | | | Intermediate group | Acquired | | |
|---|---|---|---|---|---|---|
| Items | Reactant A | Reactant B | C | amount (g) | Yield (%) | Purity (%) |
| C-1 | 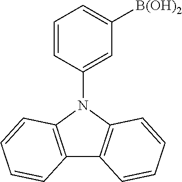 [SMILES: OB(O)c1cccc(-n2c3ccccc3c3ccccc32)c1] | 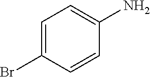 | 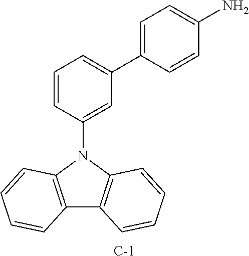 | 24.4 | 73 | 98.6 |
| C-2 | 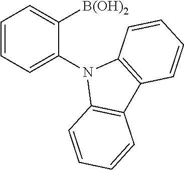 [SMILES: OB(O)c1ccccc1-n1c2ccccc2c2ccccc21] | | 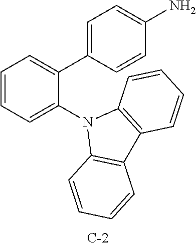 | 19.7 | 59 | 98.9 |

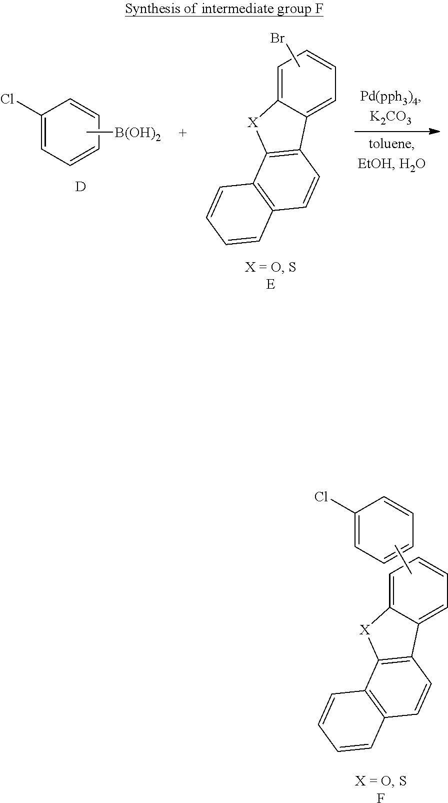

Reactant D (17.2 g, 110 mmol), reactant E (29.7 g, 100 mmol), Pd(PPh₃)₄ (2.31 g, 2.0 mmol), K₂CO₃ (27.6 g, 200 mmol) and toluene (150 mL), water (80 mL), and ethanol (50 mL) were added to a round bottom flask under nitrogen atmosphere and then stirred at 100° C. to perform reaction. When the reaction was finished, after separating an organic layer, then removing water, and performing filtering, the mixture was purified in a silica gel column and recrystallization method to manufacture intermediate group F.

The result is expressed in Table 2 below.

TABLE 2

| | | | | Acquired | | Purity |
|---|---|---|---|---|---|---|
| Items | Reactant D | Reactant E | Intermediate group F | amount (g) | Yield (%) | (%) |
| F-1 | 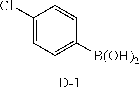 | 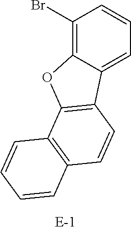 [SMILES: Brc1cccc2c1oc1c3ccccc3ccc21] | 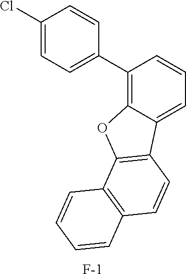 | 25.9 | 79 | 99.1 |
| F-2 | | 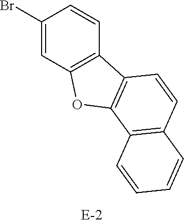 [SMILES: Brc1ccc2c(c1)oc1c3ccccc3ccc21] | 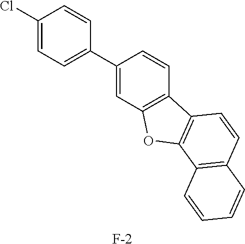 | 24.7 | 75 | 99.2 |
| F-3 | | 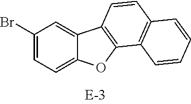 [SMILES: Brc1ccc2oc3c4ccccc4ccc3c2c1] | 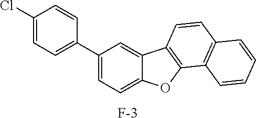 | 26.6 | 81 | 99.1 |
| F-4 | | 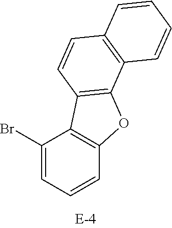 [SMILES: Brc1cccc2oc3c4ccccc4ccc3c12] | 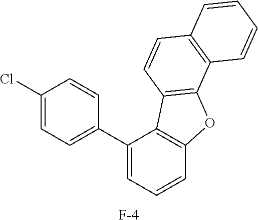 | 23.3 | 71 | 98.9 |
| F-5 | | 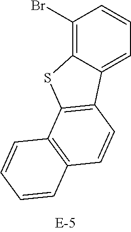 [SMILES: Brc1cccc2c1sc1c3ccccc3ccc21] | 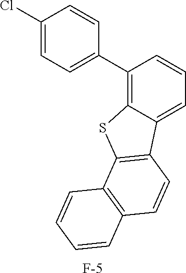 | 25.2 | 73 | 98.7 |
| F-6 | | 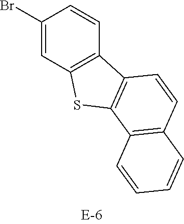 [SMILES: Brc1ccc2c(c1)sc1c3ccccc3ccc21] | 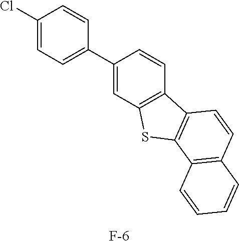 | 23.8 | 69 | 98.6 |
| F-7 | | 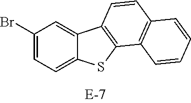 [SMILES: Brc1ccc2sc3c4ccccc4ccc3c2c1] | 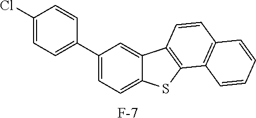 | 24.8 | 72 | 99.1 |
| F-8 | | 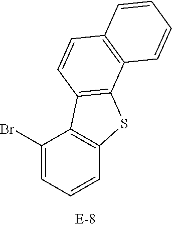 [SMILES: Brc1cccc2sc3c4ccccc4ccc3c12] | 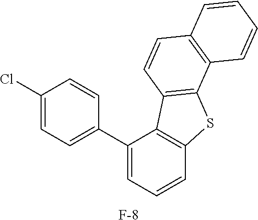 | 23.4 | 68 | 98.5 |
| F-9 | 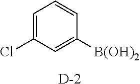 | 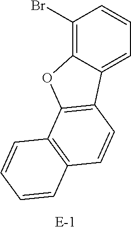 [SMILES: Brc1cccc2c1oc1c3ccccc3ccc21] | 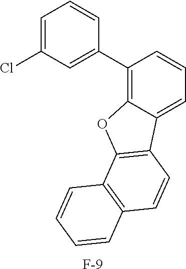 | 24.7 | 75 | 97.8 |
| F-10 | | 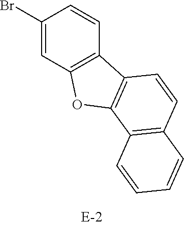 [SMILES: Brc1ccc2c(c1)oc1c3ccccc3ccc21] | 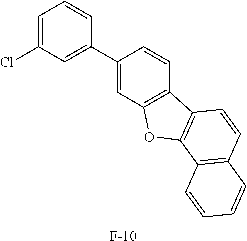 | 20.7 | 63 | 99.3 |
| F-11 | | 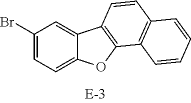 [SMILES: Brc1ccc2oc3c4ccccc4ccc3c2c1] | 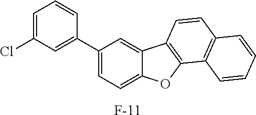 | 22.7 | 69 | 97.9 |
| F-12 | | 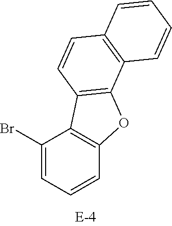 [SMILES: Brc1cccc2oc3c4ccccc4ccc3c12] | 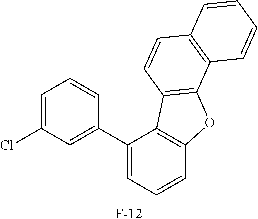 | 26.0 | 79 | 98.2 |
| F-13 | | 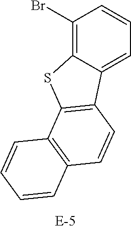 [SMILES: Brc1cccc2c1sc1c3ccccc3ccc21] | 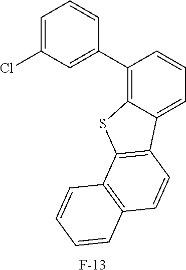 | 21.4 | 62 | 98.4 |
| F-14 | | 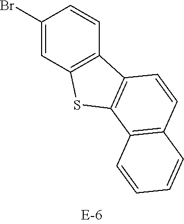 [SMILES: Brc1ccc2c(c1)sc1c3ccccc3ccc21] | 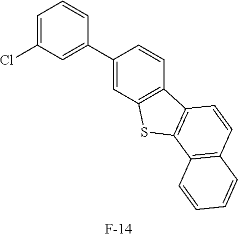 | 23.4 | 68 | 98.0 |
| F-15 | | 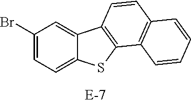 [SMILES: Brc1ccc2sc3c4ccccc4ccc3c2c1] | 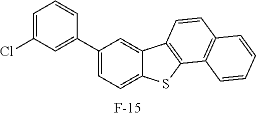 | 22.8 | 66 | 97.8 |
| F-16 | | 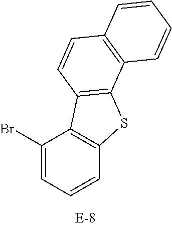 [SMILES: Brc1cccc2sc3c4ccccc4ccc3c12] | 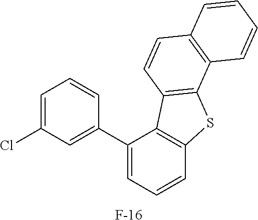 | 20.3 | 59 | 99.0 |
| F-17 | 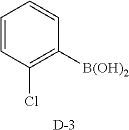 | 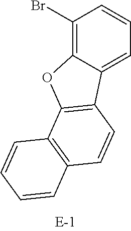 [SMILES: Brc1cccc2c1oc1c3ccccc3ccc21] | 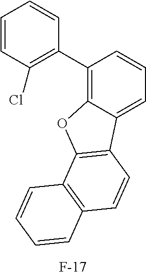 | 18.4 | 56 | 97.6 |
| F-18 | | 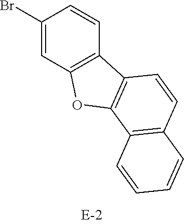 [SMILES: Brc1ccc2c(c1)oc1c3ccccc3ccc21] | 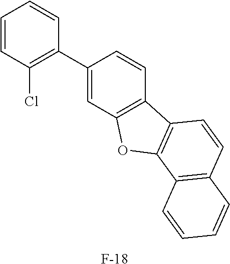 | 18.1 | 55 | 98.4 |
| F-19 | | 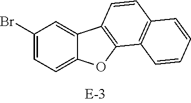 [SMILES: Brc1ccc2oc3c4ccccc4ccc3c2c1] | 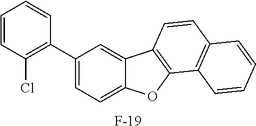 | 19.1 | 58 | 99.1 |
| F-20 | | 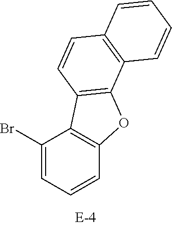 [SMILES: Brc1cccc2oc3c4ccccc4ccc3c12] | 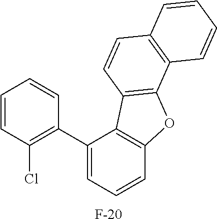 | 17.1 | 52 | 97.8 |
| F-21 | | 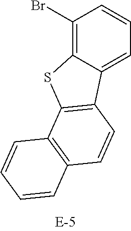 [SMILES: Brc1cccc2c1sc1c3ccccc3ccc21] | 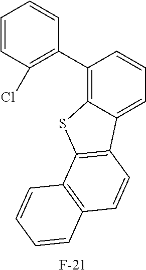 | 20.3 | 59 | 97.9 |
| F-22 | | 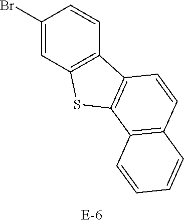 [SMILES: Brc1ccc2c(c1)sc1c3ccccc3ccc21] | 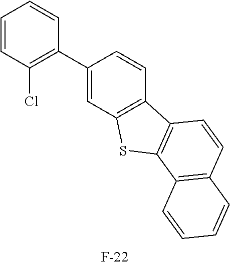 | 17.6 | 51 | 98.1 |
| F-23 | | 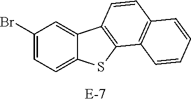 [SMILES: Brc1ccc2sc3c4ccccc4ccc3c2c1] | 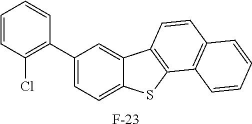 | 19.3 | 56 | 98.3 |
| F-24 | | 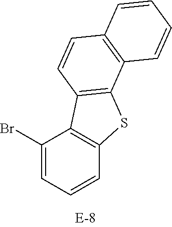 [SMILES: Brc1cccc2sc3c4ccccc4ccc3c12] | 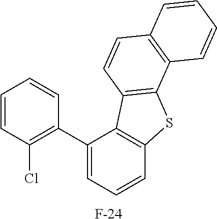 | 18.3 | 53 | 98.6 |
| F-25 | 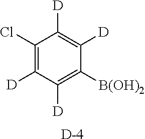 | 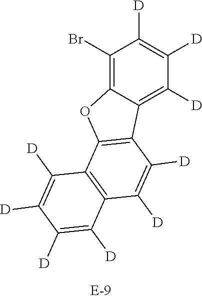 [SMILES: [2H]c1c([2H])c([2H])c2c(oc3c4c([2H])c([2H])c([2H])c([2H])c4c([2H])c([2H])c32)c1Br] | 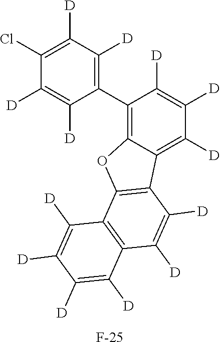 | 19.5 | 59 | 98.5 |
| F-26 | | 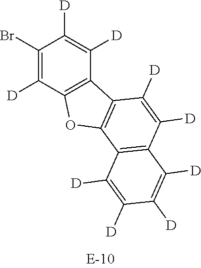 [SMILES: [2H]c1c([2H])c([2H])c2c(c1[2H])c([2H])c([2H])c1c2oc2c([2H])c(Br)c([2H])c([2H])c21] | 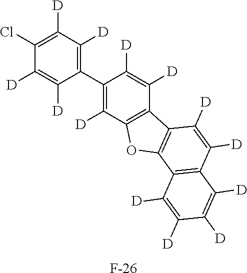 | 19.8 | 62 | 97.9 |
| F-27 | | 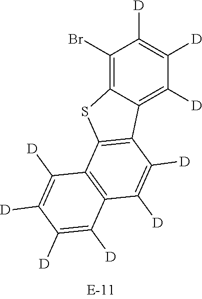 [SMILES: [2H]c1c([2H])c([2H])c2c(sc3c4c([2H])c([2H])c([2H])c([2H])c4c([2H])c([2H])c32)c1Br] | 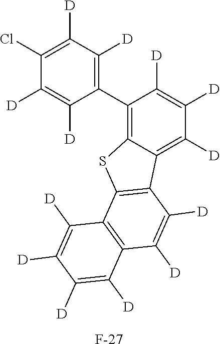 | 17.8 | 52 | 98.5 |

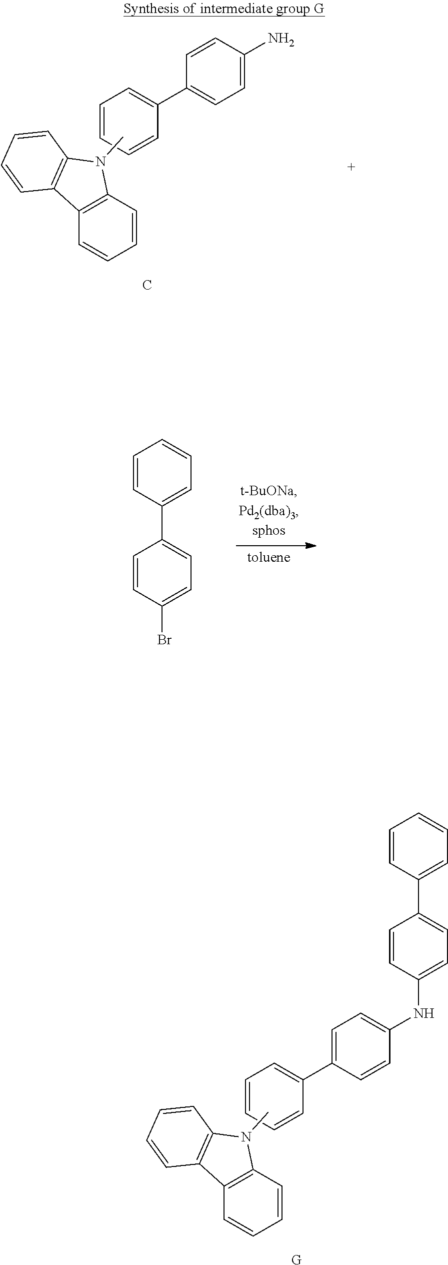

Intermediate C (33.4 g, 100 mmol), 4-bromobiphenyl (25.6 g, 110 mmol), Pd₂(dba)₃ (1.83 g, 2.0 mmol), t-BuONa (19.2 g, 200 mmol), SPhos (1.64 g, 4.0 mmol), and toluene (480 mL) were added to a round bottom flask under nitrogen atmosphere and then stirred at 100° C. to perform the reaction. When the reaction was finished, the mixture was precipitated in methanol and after the precipitate was filtered, purified in a silica gel column and recrystallization method to manufacture intermediate group G.

The result is expressed in Table 3 below.

TABLE 3

| | Intermediate | | Intermediate | | Yield | Purity |
|---|---|---|---|---|---|---|
| Items | group C | Reactant | group G | Acquired amount (g) | (%) | (%) |
| G-1 | 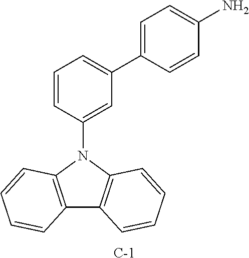 | 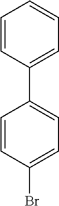 [SMILES: Brc1ccc(-c2ccccc2)cc1] | 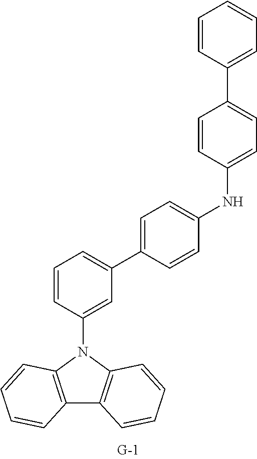 | 37.0 | 76 | 99.3 |
| G-2 | 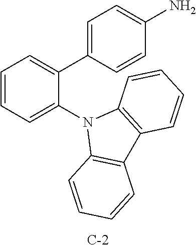 | | 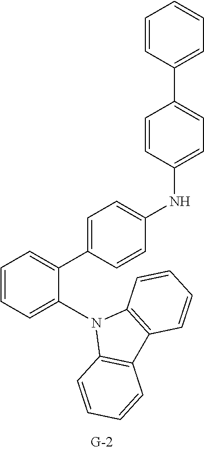 | 38.4 | 79 | 99.5 |

### Synthesis of Compound Group H

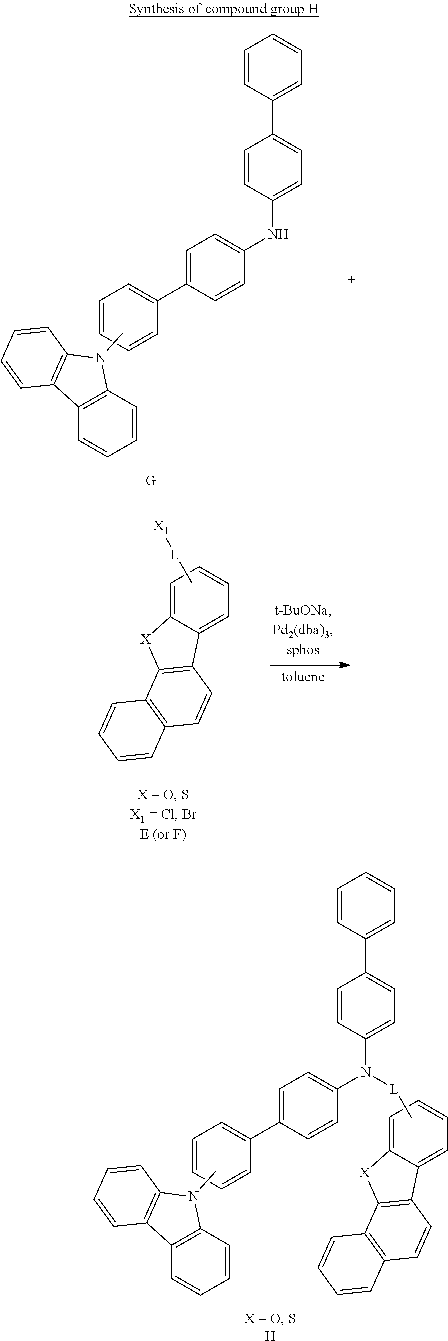

Intermediate G (4.86 g, 10 mmol), intermediate E (or F) (11 mmol), Pd₂(dba)₃ (0.183 g, 0.2 mmol), t-BuONa (1.92 g, 20 mmol), SPhos (0.164 g, 0.4 mmol), and toluene (60 mL) were added to a round bottom flask under nitrogen atmosphere and then stirred at 100° C. to perform the reaction. When the reaction was finished, the mixture was precipitated in methanol and after the precipitate was filtered, purified in a silica gel column and recrystallization method to manufacture compound group H.

The result is expressed in Table 4 below.

TABLE 4

| | | Intermediate | | Acquired | | | MS |
|---|---|---|---|---|---|---|---|
| | Intermediate | groups | Compound group | amount | Yield | Purity | [M + |
| Items | group G | E and F | H | (g) | (%) | (%) | H]+ |
| Com- pound 1-97 | 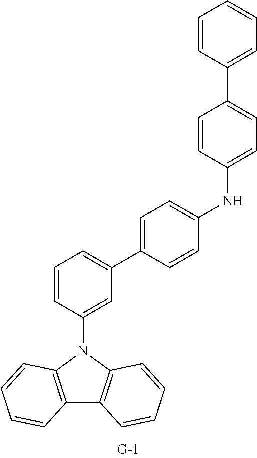 [SMILES: c1ccc(-c2ccc(Nc3ccc(-c4cccc(-n5c6ccccc6c6ccccc65)c4)cc3)cc2)cc1] | 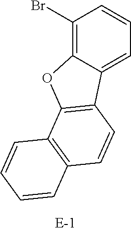 [SMILES: Brc1cccc2c1oc1c3ccccc3ccc21] | 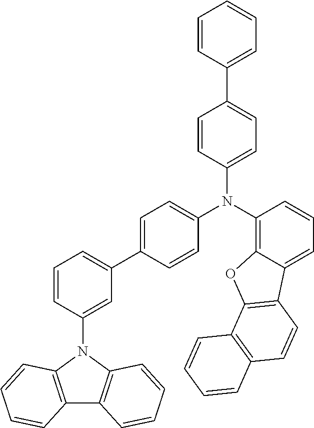 [SMILES: c1ccc(-c2ccc(N(c3ccc(-c4cccc(-n5c6ccccc6c6ccccc65)c4)cc3)c3cccc4c3oc3c5ccccc5ccc43)cc2)cc1] | 4.56 | 65 | 99.96 | 703.27 |
| Com- pound 1-103 | | 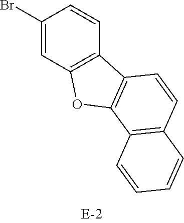 [SMILES: Brc1ccc2c(c1)oc1c3ccccc3ccc21] | 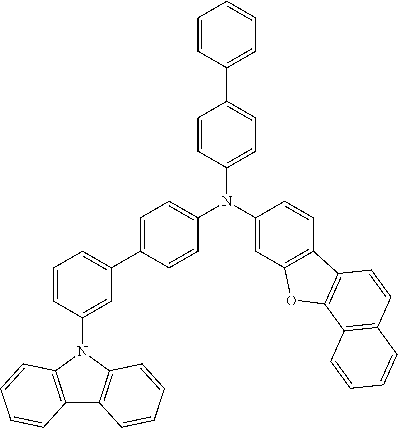 [SMILES: c1ccc(-c2ccc(N(c3ccc(-c4cccc(-n5c6ccccc6c6ccccc65)c4)cc3)c3ccc4c(c3)oc3c5ccccc5ccc43)cc2)cc1] | 4.77 | 68 | 99.95 | 703.27 |
| Com- pound 1-109 | | 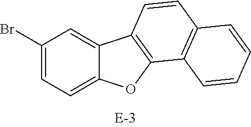 [SMILES: Brc1ccc2oc3c4ccccc4ccc3c2c1] | 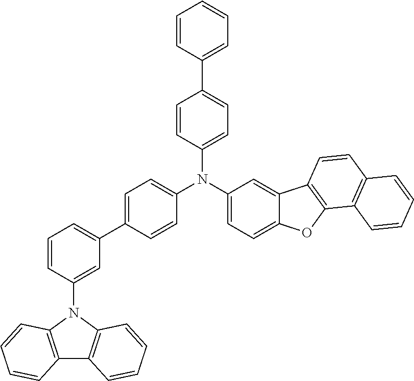 [SMILES: c1ccc(-c2ccc(N(c3ccc(-c4cccc(-n5c6ccccc6c6ccccc65)c4)cc3)c3ccc4oc5c6ccccc6ccc5c4c3)cc2)cc1] | 4.14 | 59 | 99.93 | 703.28 |
| Com- pound 1-115 | | 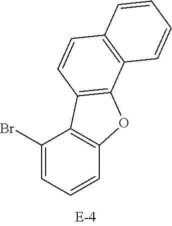 [SMILES: Brc1cccc2oc3c4ccccc4ccc3c12] | 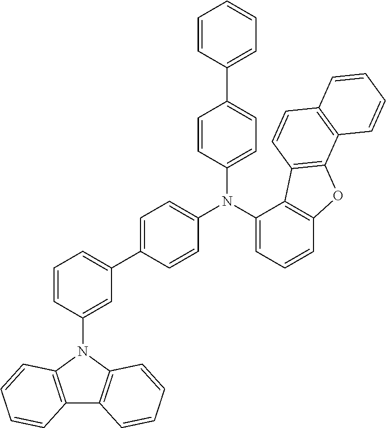 [SMILES: c1ccc(-c2ccc(N(c3ccc(-c4cccc(-n5c6ccccc6c6ccccc65)c4)cc3)c3cccc4oc5c6ccccc6ccc5c34)cc2)cc1] | 4.84 | 69 | 99.94 | 703.27 |
| Com- pound 1-809 | | 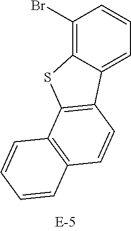 [SMILES: Brc1cccc2c1sc1c3ccccc3ccc21] | 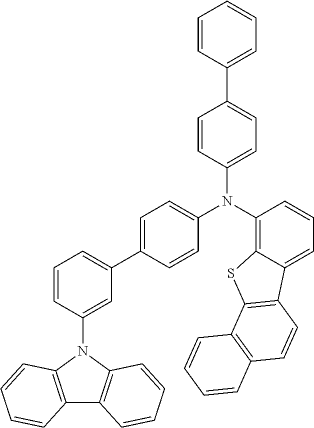 [SMILES: c1ccc(-c2ccc(N(c3ccc(-c4cccc(-n5c6ccccc6c6ccccc65)c4)cc3)c3cccc4c3sc3c5ccccc5ccc43)cc2)cc1] | 5.17 | 72 | 99.92 | 719.25 |
| Com- pound 1-815 | | 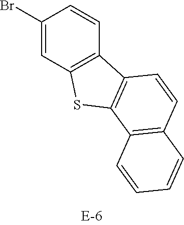 [SMILES: Brc1ccc2c(c1)sc1c3ccccc3ccc21] | 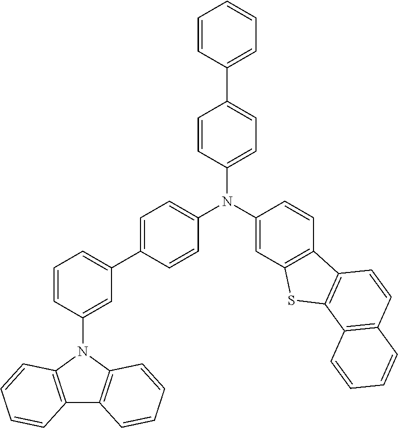 [SMILES: c1ccc(-c2ccc(N(c3ccc(-c4cccc(-n5c6ccccc6c6ccccc65)c4)cc3)c3ccc4c(c3)sc3c5ccccc5ccc43)cc2)cc1] | 5.02 | 70 | 99.97 | 719.24 |
| Com- pound 1-121 | | 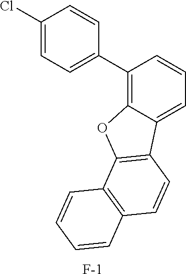 | 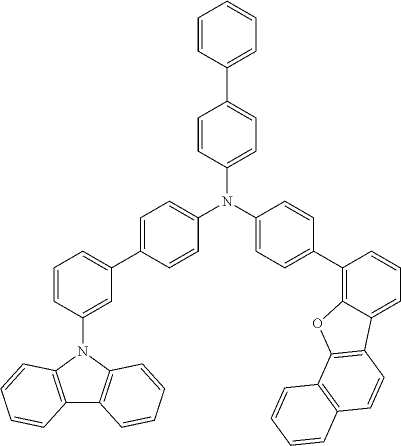 [SMILES: c1ccc(-c2ccc(N(c3ccc(-c4cccc(-n5c6ccccc6c6ccccc65)c4)cc3)c3ccc(-c4cccc5c4oc4c6ccccc6ccc54)cc3)cc2)cc1] | 5.84 | 75 | 99.91 | 779.30 |
| Com- pound 1-127 | | 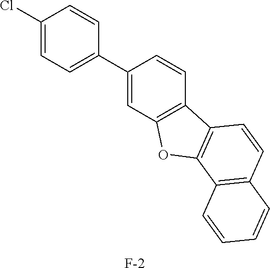 | 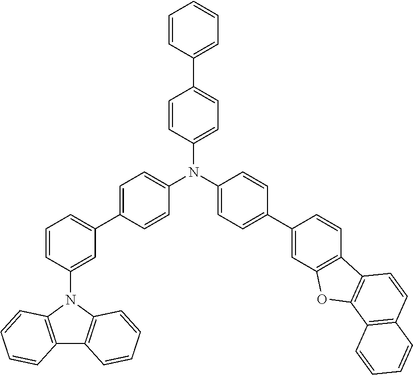 [SMILES: c1ccc(-c2ccc(N(c3ccc(-c4cccc(-n5c6ccccc6c6ccccc65)c4)cc3)c3ccc(-c4ccc5c(c4)oc4c6ccccc6ccc54)cc3)cc2)cc1] | 5.60 | 72 | 99.95 | 779.30 |
| Com- pound 1-133 | | 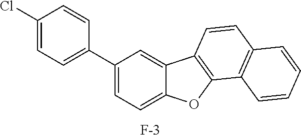 | 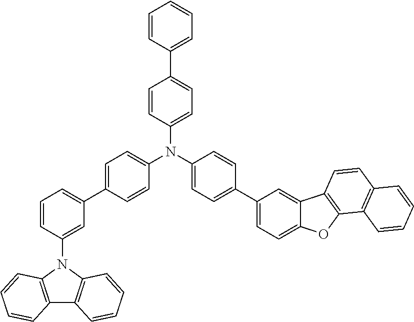 [SMILES: c1ccc(-c2ccc(N(c3ccc(-c4cccc(-n5c6ccccc6c6ccccc65)c4)cc3)c3ccc(-c4ccc5oc6c7ccccc7ccc6c5c4)cc3)cc2)cc1] | 5.37 | 69 | 99.92 | 779.29 |
| Com- pound 1-139 | | 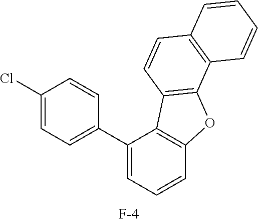 | 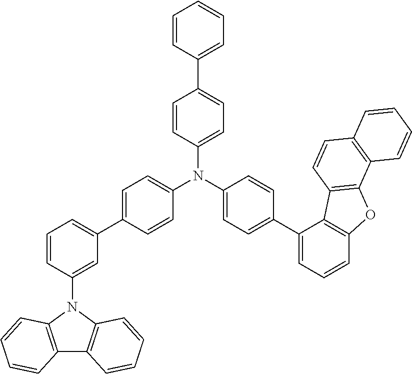 [SMILES: c1ccc(-c2ccc(N(c3ccc(-c4cccc(-n5c6ccccc6c6ccccc65)c4)cc3)c3ccc(-c4cccc5oc6c7ccccc7ccc6c45)cc3)cc2)cc1] | 4.51 | 58 | 99.94 | 779.30 |
| Com- pound 1-821 | | 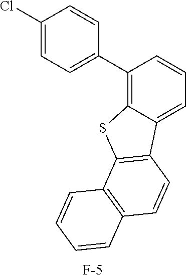 | 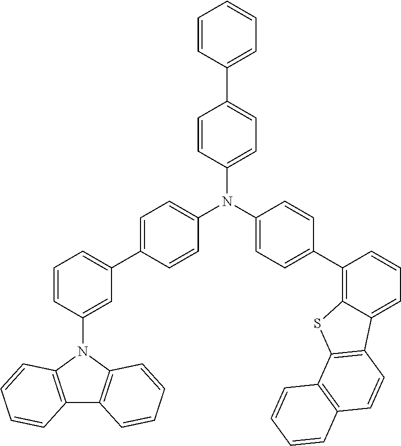 [SMILES: c1ccc(-c2ccc(N(c3ccc(-c4cccc(-n5c6ccccc6c6ccccc65)c4)cc3)c3ccc(-c4cccc5c4sc4c6ccccc6ccc54)cc3)cc2)cc1] | 6.20 | 78 | 99.98 | 795.27 |
| Com- pound 1-827 | | 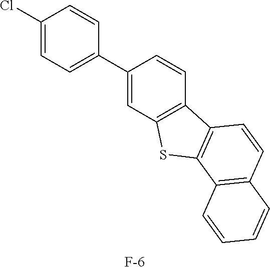 | 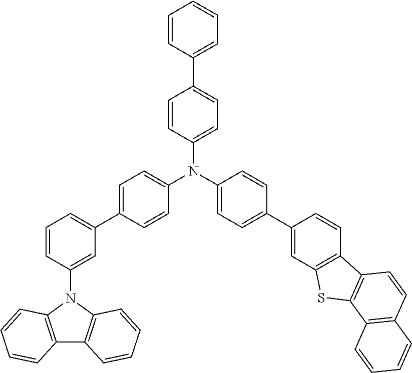 [SMILES: c1ccc(-c2ccc(N(c3ccc(-c4cccc(-n5c6ccccc6c6ccccc65)c4)cc3)c3ccc(-c4ccc5c(c4)sc4c6ccccc6ccc54)cc3)cc2)cc1] | 5.88 | 74 | 99.96 | 795.28 |
| Com- pound 1-145 | | 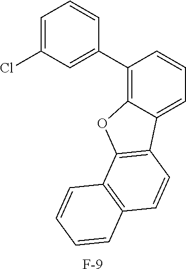 | 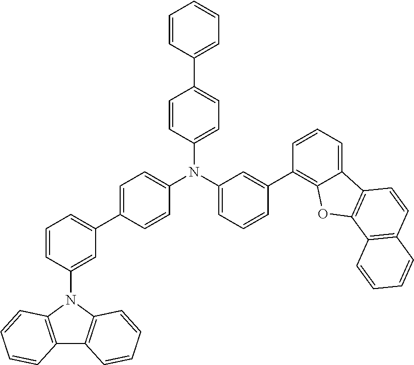 [SMILES: c1ccc(-c2ccc(N(c3ccc(-c4cccc(-n5c6ccccc6c6ccccc65)c4)cc3)c3cccc(-c4cccc5c4oc4c6ccccc6ccc54)c3)cc2)cc1] | 4.98 | 64 | 99.92 | 779.30 |
| Com- pound 1-151 | | 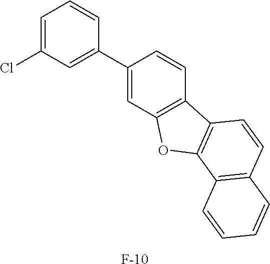 | 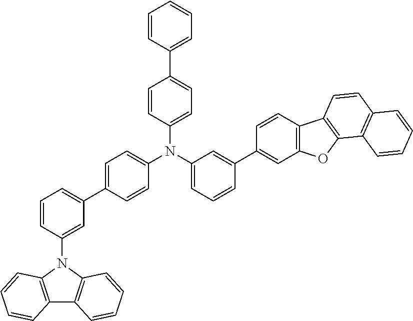 [SMILES: c1ccc(-c2ccc(N(c3ccc(-c4cccc(-n5c6ccccc6c6ccccc65)c4)cc3)c3cccc(-c4ccc5c(c4)oc4c6ccccc6ccc54)c3)cc2)cc1] | 5.14 | 66 | 99.98 | 779.29 |
| Com- pound 1-157 | | 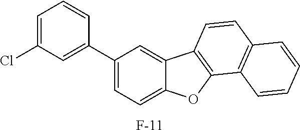 | 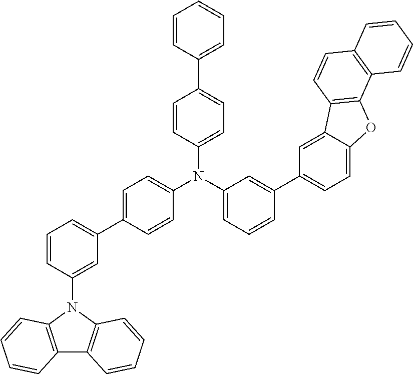 [SMILES: c1ccc(-c2ccc(N(c3ccc(-c4cccc(-n5c6ccccc6c6ccccc65)c4)cc3)c3cccc(-c4ccc5oc6c7ccccc7ccc6c5c4)c3)cc2)cc1] | 4.51 | 58 | 99.97 | 779.30 |
| Com- pound 1-163 | | 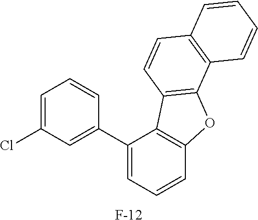 | 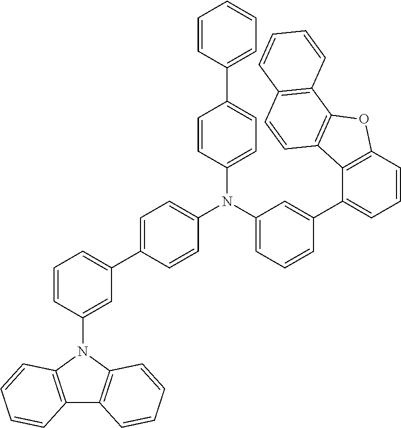 [SMILES: c1ccc(-c2ccc(N(c3ccc(-c4cccc(-n5c6ccccc6c6ccccc65)c4)cc3)c3cccc(-c4cccc5oc6c7ccccc7ccc6c45)c3)cc2)cc1] | 4.05 | 52 | 99.90 | 779.30 |
| Com- pound 1-169 | | 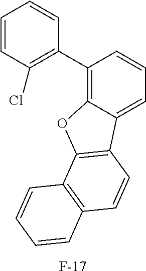 | 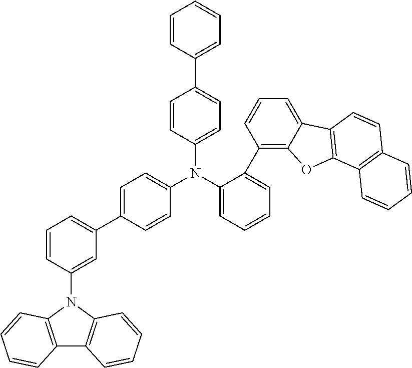 [SMILES: c1ccc(-c2ccc(N(c3ccc(-c4cccc(-n5c6ccccc6c6ccccc65)c4)cc3)c3ccccc3-c3cccc4c3oc3c5ccccc5ccc43)cc2)cc1] | 4.36 | 56 | 99.93 | 779.31 |
| Com- pound 1-175 | | 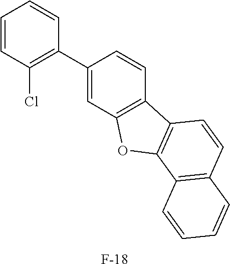 | 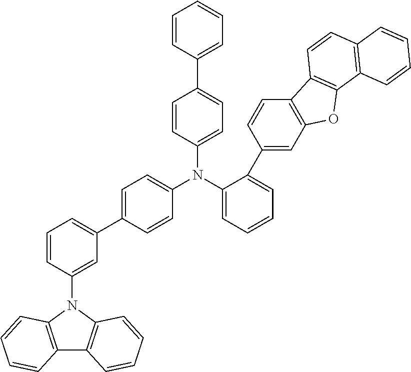 [SMILES: c1ccc(-c2ccc(N(c3ccc(-c4cccc(-n5c6ccccc6c6ccccc65)c4)cc3)c3ccccc3-c3ccc4c(c3)oc3c5ccccc5ccc43)cc2)cc1] | 4.20 | 54 | 99.92 | 779.30 |
| Com- pound 1-181 | | 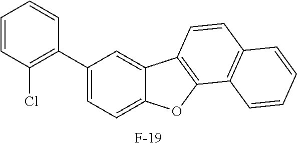 | 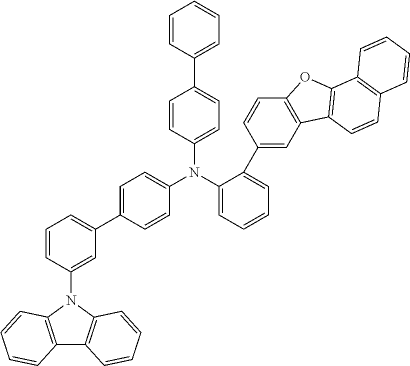 [SMILES: c1ccc(-c2ccc(N(c3ccc(-c4cccc(-n5c6ccccc6c6ccccc65)c4)cc3)c3ccccc3-c3ccc4oc5c6ccccc6ccc5c4c3)cc2)cc1] | 4.51 | 58 | 99.90 | 779.30 |
| Com- pound 1-187 | | 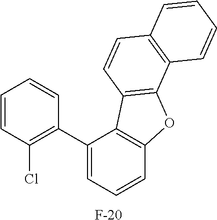 | 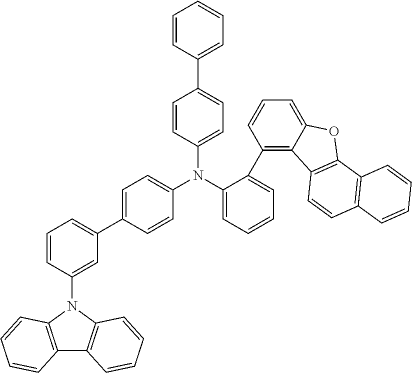 [SMILES: c1ccc(-c2ccc(N(c3ccc(-c4cccc(-n5c6ccccc6c6ccccc65)c4)cc3)c3ccccc3-c3cccc4oc5c6ccccc6ccc5c34)cc2)cc1] | 3.81 | 49 | 99.94 | 779.31 |
| Com- pound 2-25 | | 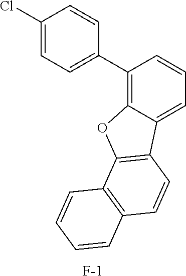 | 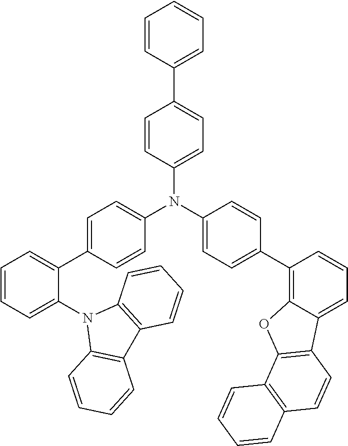 [SMILES: c1ccc(-c2ccc(N(c3ccc(-c4ccccc4-n4c5ccccc5c5ccccc54)cc3)c3ccc(-c4cccc5c4oc4c6ccccc6ccc54)cc3)cc2)cc1] | 5.45 | 70 | 99.98 | 779.30 |
| Com- pound 2-37 | 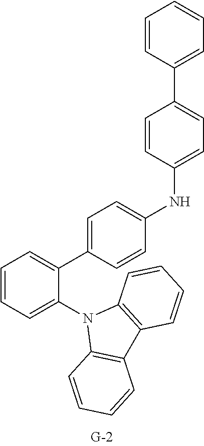 [SMILES: c1ccc(-c2ccc(Nc3ccc(-c4ccccc4-n4c5ccccc5c5ccccc54)cc3)cc2)cc1] | 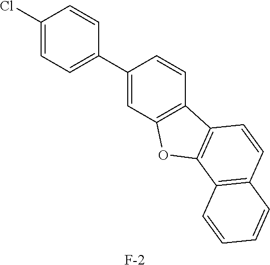 | 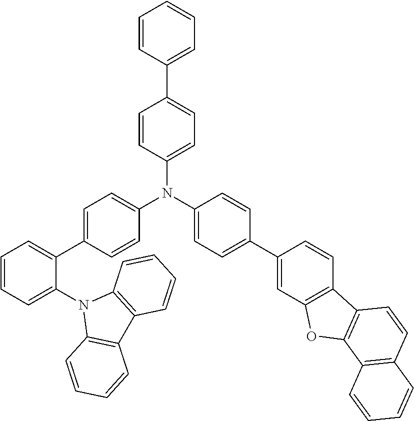 [SMILES: c1ccc(-c2ccc(N(c3ccc(-c4ccc5c(c4)oc4c6ccccc6ccc54)cc3)c3ccc(-c4ccccc4-n4c5ccccc5c5ccccc54)cc3)cc2)cc1] | 5.84 | 75 | 99.95 | 779.30 |
| Com- pound 2-31 | | 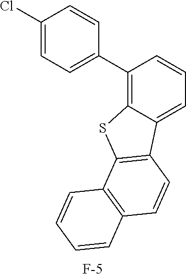 | 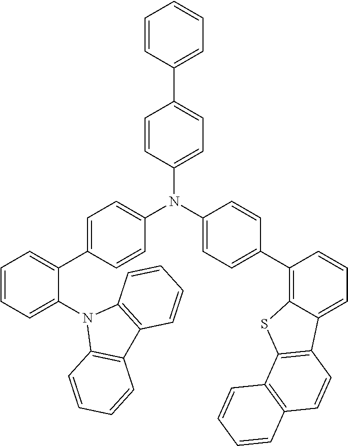 [SMILES: c1ccc(-c2ccc(N(c3ccc(-c4ccccc4-n4c5ccccc5c5ccccc54)cc3)c3ccc(-c4cccc5c4sc4c6ccccc6ccc54)cc3)cc2)cc1] | 6.20 | 78 | 99.97 | 795.28 |
| Com- pound 2-43 | | 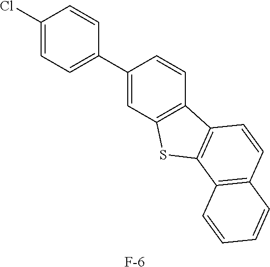 | 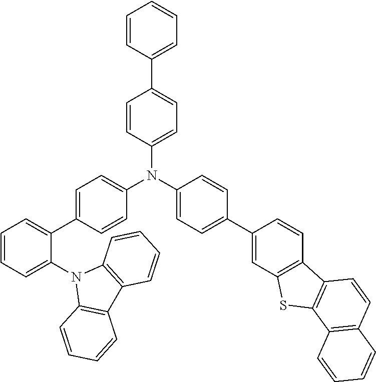 [SMILES: c1ccc(-c2ccc(N(c3ccc(-c4ccc5c(c4)sc4c6ccccc6ccc54)cc3)c3ccc(-c4ccccc4-n4c5ccccc5c5ccccc54)cc3)cc2)cc1] | 5.72 | 72 | 99.96 | 795.27 |

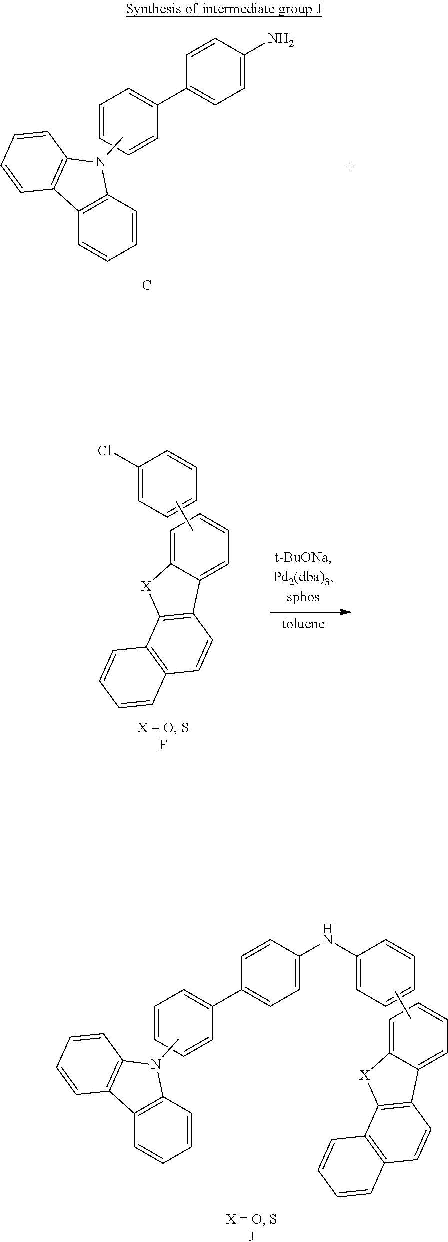

Intermediate C (33.4 g, 100 mmol), intermediate F (36.2 g, 110 mmol), Pd₂(dba)₃ (1.83 g, 2.0 mmol), t-BuONa (19.2 g, 200 mmol), SPhos (1.64 g, 4.0 mmol), and toluene (560 mL) were added to a round bottom flask under nitrogen atmosphere and then stirred at 100° C. to perform the reaction. When the reaction was finished, the mixture was precipitated in methanol and after the precipitate was filtered, purified in a silica gel column and recrystallization method to manufacture intermediate group J.

The result is expressed in Table 5 below.

TABLE 5

| | | | | Acquired | | |
|---|---|---|---|---|---|---|
| | Intermediate | Intermediate | | amount | Yield | Purity |
| Items | group C | group F | Intermediate group J | (g) | (%) | (%) |
| J-1 | 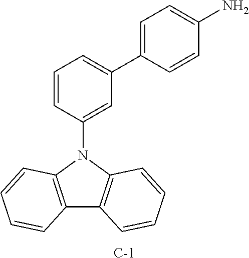 | 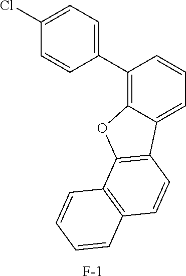 | 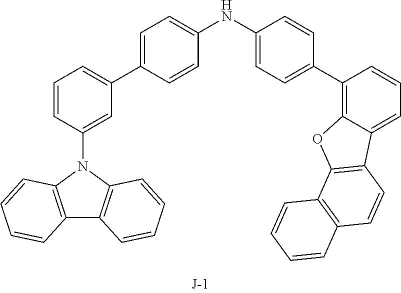 [SMILES: c1cc(-c2ccc(Nc3ccc(-c4cccc5c4oc4c6ccccc6ccc54)cc3)cc2)cc(-n2c3ccccc3c3ccccc32)c1] | 40.0 | 64 | 99.3 |
| J-2 | | 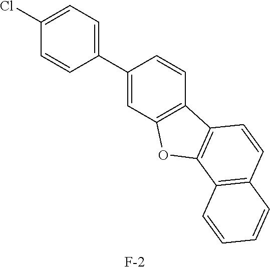 | 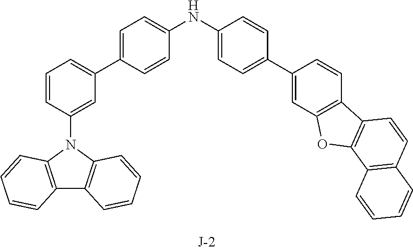 [SMILES: c1cc(-c2ccc(Nc3ccc(-c4ccc5c(c4)oc4c6ccccc6ccc54)cc3)cc2)cc(-n2c3ccccc3c3ccccc32)c1] | 44.4 | 71 | 99.2 |
| J-3 | | 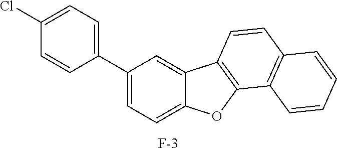 | 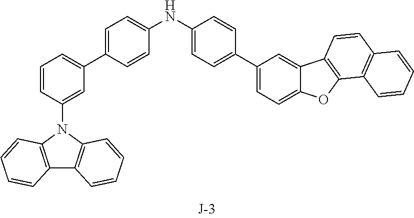 [SMILES: c1cc(-c2ccc(Nc3ccc(-c4ccc5oc6c7ccccc7ccc6c5c4)cc3)cc2)cc(-n2c3ccccc3c3ccccc32)c1] | 36.9 | 59 | 99.1 |
| J-4 | | 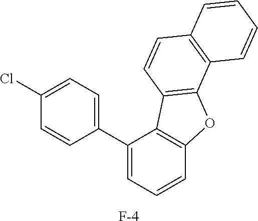 | 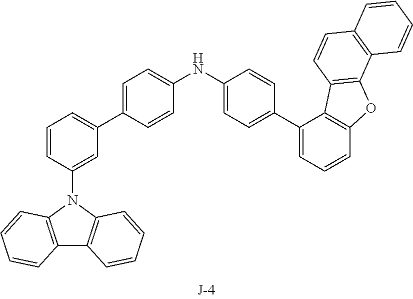 [SMILES: c1cc(-c2ccc(Nc3ccc(-c4cccc5oc6c7ccccc7ccc6c45)cc3)cc2)cc(-n2c3ccccc3c3ccccc32)c1] | 45.0 | 72 | 99.3 |
| J-5 | | 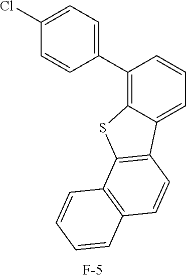 | 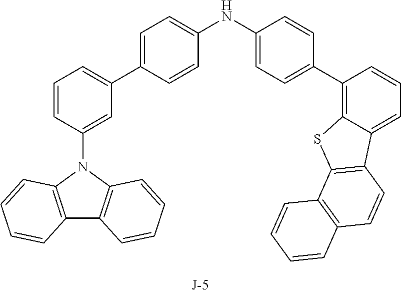 [SMILES: c1cc(-c2ccc(Nc3ccc(-c4cccc5c4sc4c6ccccc6ccc54)cc3)cc2)cc(-n2c3ccccc3c3ccccc32)c1] | 43.65 | 68 | 99.5 |
| J-6 | | 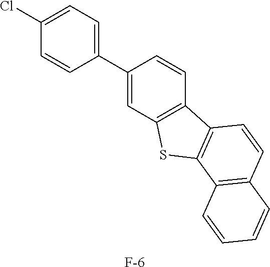 | 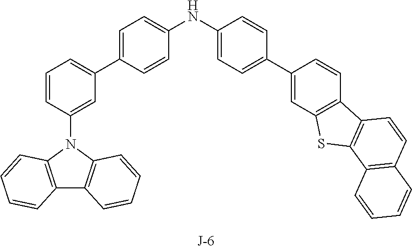 [SMILES: c1cc(-c2ccc(Nc3ccc(-c4ccc5c(c4)sc4c6ccccc6ccc54)cc3)cc2)cc(-n2c3ccccc3c3ccccc32)c1] | 39.1 | 61 | 99.2 |
| J-7 | | 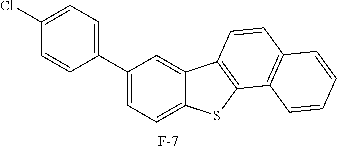 | 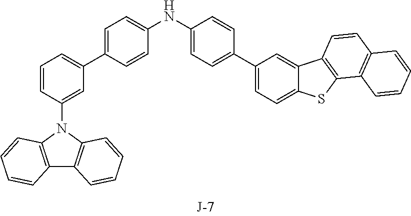 [SMILES: c1cc(-c2ccc(Nc3ccc(-c4ccc5sc6c7ccccc7ccc6c5c4)cc3)cc2)cc(-n2c3ccccc3c3ccccc32)c1] | 38.5 | 60 | 99.0 |
| J-8 | | 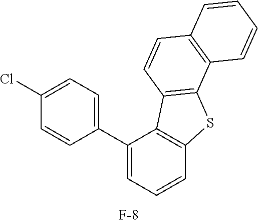 | 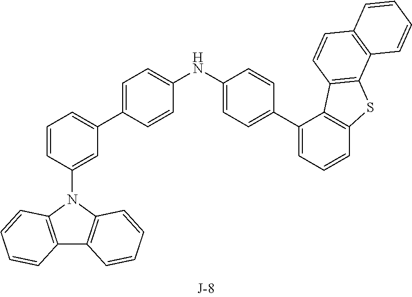 [SMILES: c1cc(-c2ccc(Nc3ccc(-c4cccc5sc6c7ccccc7ccc6c45)cc3)cc2)cc(-n2c3ccccc3c3ccccc32)c1] | 46.8 | 73 | 99.5 |
| J-9 | | 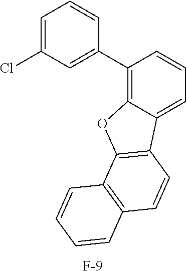 | 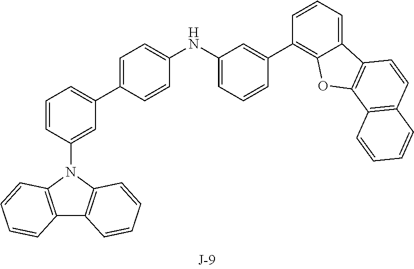 [SMILES: c1cc(Nc2ccc(-c3cccc(-n4c5ccccc5c5ccccc54)c3)cc2)cc(-c2cccc3c2oc2c4ccccc4ccc32)c1] | 36.9 | 59 | 99.4 |
| J-10 | | 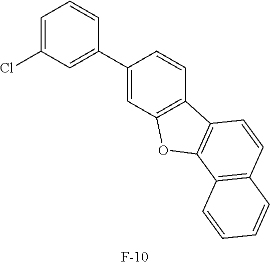 | 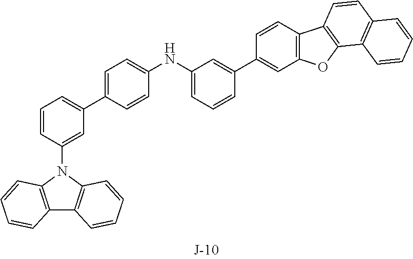 [SMILES: c1cc(Nc2ccc(-c3cccc(-n4c5ccccc5c5ccccc54)c3)cc2)cc(-c2ccc3c(c2)oc2c4ccccc4ccc32)c1] | 39.4 | 63 | 99.2 |
| J-11 | | 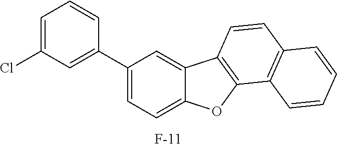 | 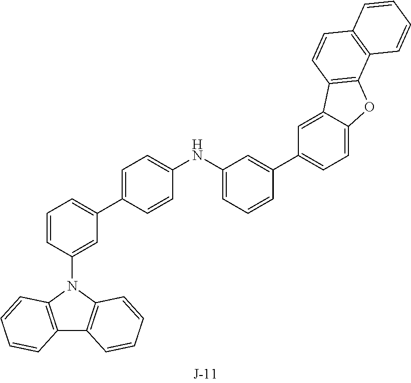 [SMILES: c1cc(Nc2ccc(-c3cccc(-n4c5ccccc5c5ccccc54)c3)cc2)cc(-c2ccc3oc4c5ccccc5ccc4c3c2)c1] | 41.3 | 66 | 99.6 |
| J-12 | | 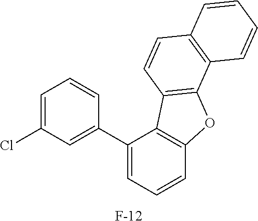 | 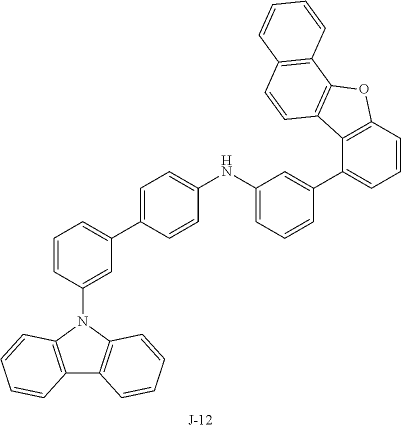 [SMILES: c1cc(Nc2ccc(-c3cccc(-n4c5ccccc5c5ccccc54)c3)cc2)cc(-c2cccc3oc4c5ccccc5ccc4c23)c1] | 42.5 | 68 | 99.4 |
| J-13 | | 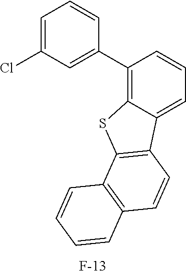 | 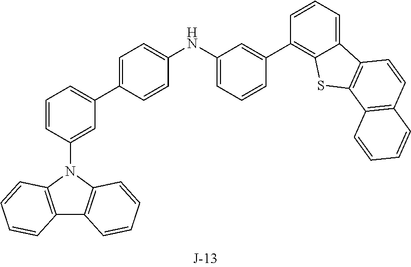 [SMILES: c1cc(Nc2ccc(-c3cccc(-n4c5ccccc5c5ccccc54)c3)cc2)cc(-c2cccc3c2sc2c4ccccc4ccc32)c1] | 40.4 | 63 | 99.0 |
| J-14 | | 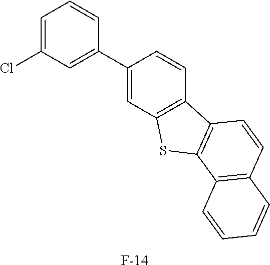 | 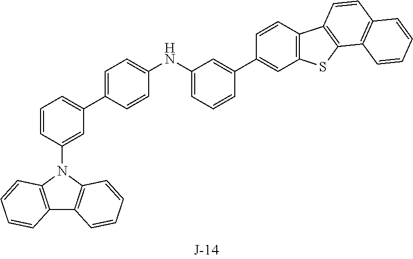 [SMILES: c1cc(Nc2ccc(-c3cccc(-n4c5ccccc5c5ccccc54)c3)cc2)cc(-c2ccc3c(c2)sc2c4ccccc4ccc32)c1] | 41.0 | 64 | 99.3 |
| J-15 | | 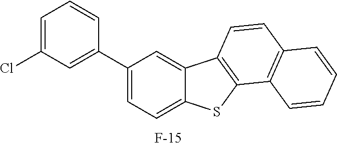 | 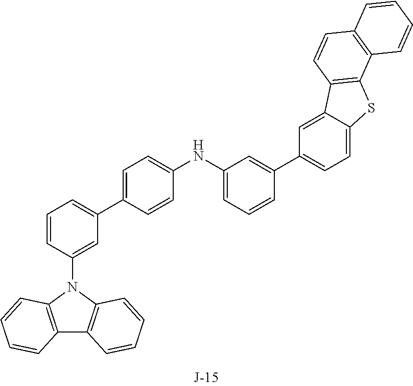 [SMILES: c1cc(Nc2ccc(-c3cccc(-n4c5ccccc5c5ccccc54)c3)cc2)cc(-c2ccc3sc4c5ccccc5ccc4c3c2)c1] | 43.6 | 68 | 99.1 |
| J-16 | | 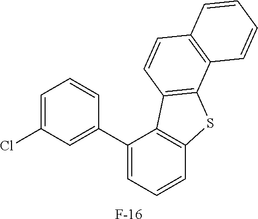 | 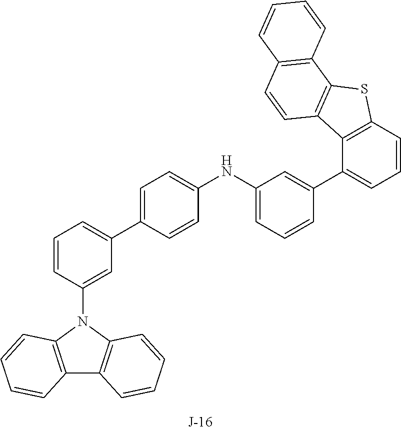 [SMILES: c1cc(Nc2ccc(-c3cccc(-n4c5ccccc5c5ccccc54)c3)cc2)cc(-c2cccc3sc4c5ccccc5ccc4c23)c1] | 45.5 | 71 | 99.4 |
| J-17 | | 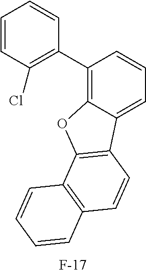 | 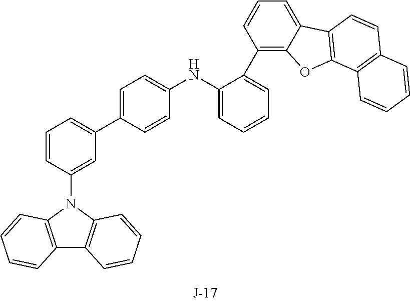 [SMILES: c1cc(-c2ccc(Nc3ccccc3-c3cccc4c3oc3c5ccccc5ccc43)cc2)cc(-n2c3ccccc3c3ccccc32)c1] | 43.8 | 70 | 99.0 |
| J-18 | | 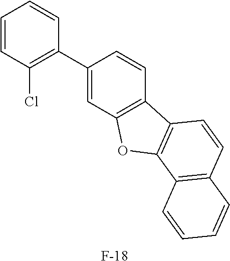 | 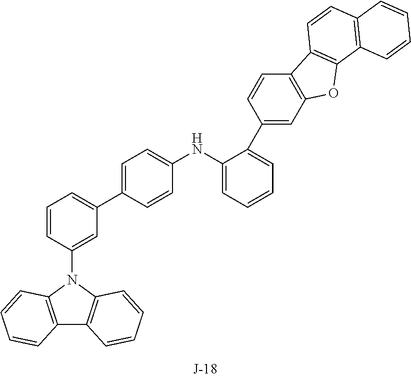 [SMILES: c1cc(-c2ccc(Nc3ccccc3-c3ccc4c(c3)oc3c5ccccc5ccc43)cc2)cc(-n2c3ccccc3c3ccccc32)c1] | 36.9 | 59 | 99.1 |
| J-19 | | 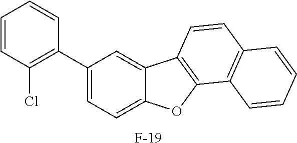 | 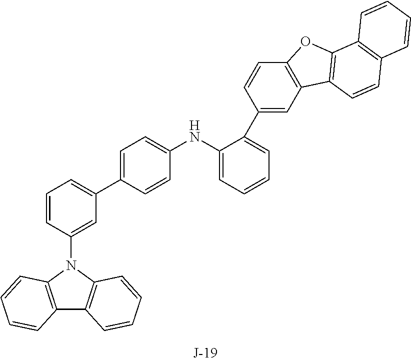 [SMILES: c1cc(-c2ccc(Nc3ccccc3-c3ccc4oc5c6ccccc6ccc5c4c3)cc2)cc(-n2c3ccccc3c3ccccc32)c1] | 40.7 | 65 | 99.0 |
| J-20 | | 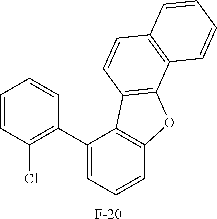 | 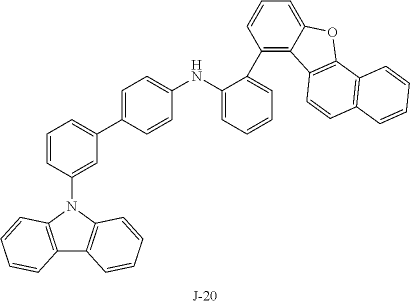 [SMILES: c1cc(-c2ccc(Nc3ccccc3-c3cccc4oc5c6ccccc6ccc5c34)cc2)cc(-n2c3ccccc3c3ccccc32)c1] | 38.2 | 61 | 99.3 |
| J-21 | | 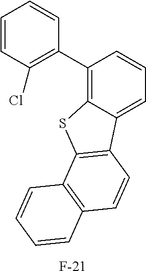 | 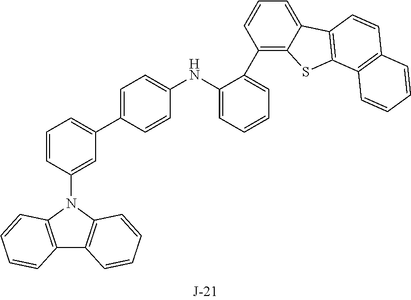 [SMILES: c1cc(-c2ccc(Nc3ccccc3-c3cccc4c3sc3c5ccccc5ccc43)cc2)cc(-n2c3ccccc3c3ccccc32)c1] | 38.5 | 60 | 99.5 |
| J-22 | | 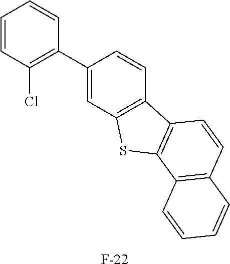 | 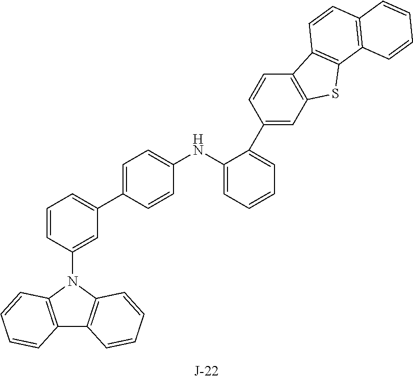 [SMILES: c1cc(-c2ccc(Nc3ccccc3-c3ccc4c(c3)sc3c5ccccc5ccc43)cc2)cc(-n2c3ccccc3c3ccccc32)c1] | 37.2 | 58 | 99.4 |
| J-23 | | 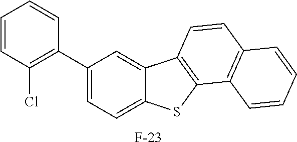 | 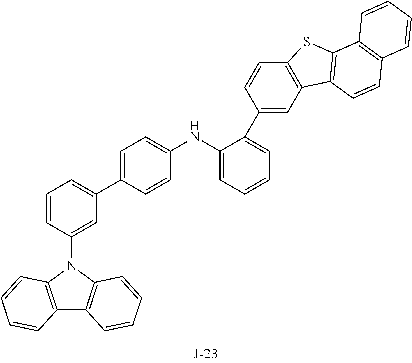 [SMILES: c1cc(-c2ccc(Nc3ccccc3-c3ccc4sc5c6ccccc6ccc5c4c3)cc2)cc(-n2c3ccccc3c3ccccc32)c1] | 35.9 | 56 | 99.3 |
| J-24 | | 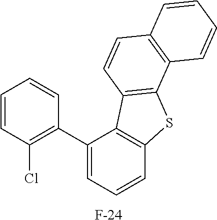 | 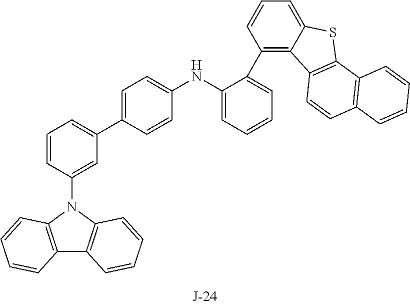 [SMILES: c1cc(-c2ccc(Nc3ccccc3-c3cccc4sc5c6ccccc6ccc5c34)cc2)cc(-n2c3ccccc3c3ccccc32)c1] | 38.5 | 60 | 99.0 |
| J-25 | 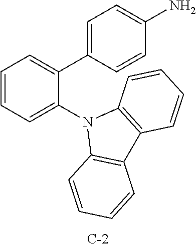 | 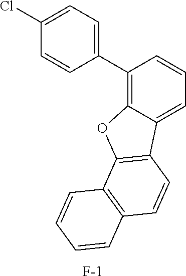 | 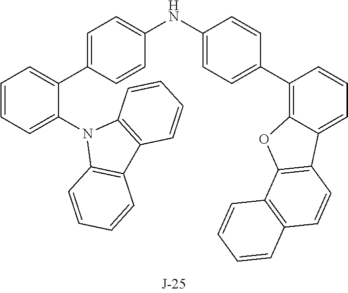 [SMILES: c1ccc(-n2c3ccccc3c3ccccc32)c(-c2ccc(Nc3ccc(-c4cccc5c4oc4c6ccccc6ccc54)cc3)cc2)c1] | 45.7 | 73 | 99.2 |
| J-26 | | 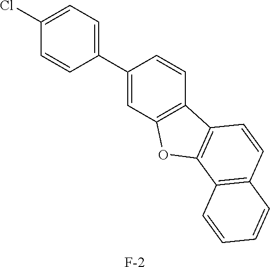 | 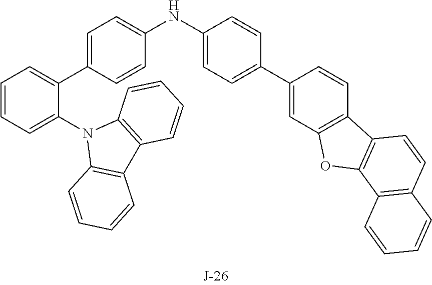 [SMILES: c1ccc(-n2c3ccccc3c3ccccc32)c(-c2ccc(Nc3ccc(-c4ccc5c(c4)oc4c6ccccc6ccc54)cc3)cc2)c1] | 43.2 | 69 | 99.5 |
| J-27 | | 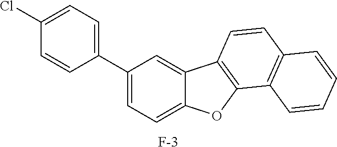 | 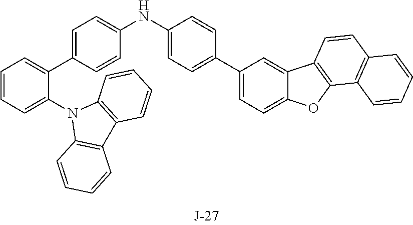 [SMILES: c1ccc(-n2c3ccccc3c3ccccc32)c(-c2ccc(Nc3ccc(-c4ccc5oc6c7ccccc7ccc6c5c4)cc3)cc2)c1] | 42.6 | 68 | 99.2 |
| J-28 | | 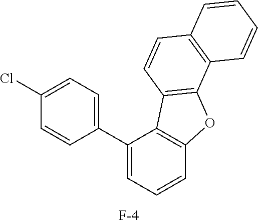 | 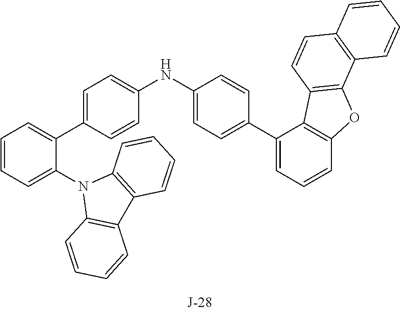 [SMILES: c1ccc(-n2c3ccccc3c3ccccc32)c(-c2ccc(Nc3ccc(-c4cccc5oc6c7ccccc7ccc6c45)cc3)cc2)c1] | 41.3 | 66 | 99.0 |
| J-29 | | 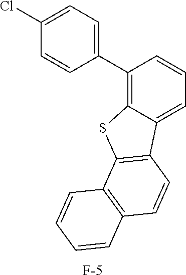 | 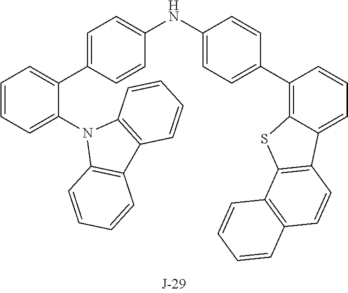 [SMILES: c1ccc(-n2c3ccccc3c3ccccc32)c(-c2ccc(Nc3ccc(-c4cccc5c4sc4c6ccccc6ccc54)cc3)cc2)c1] | 48.8 | 76 | 99.3 |
| J-30 | | 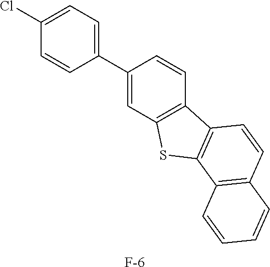 | 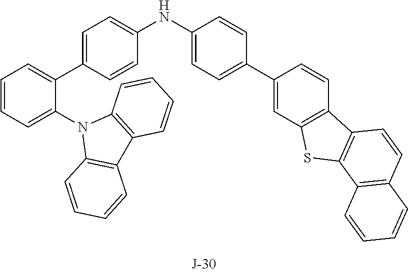 [SMILES: c1ccc(-n2c3ccccc3c3ccccc32)c(-c2ccc(Nc3ccc(-c4ccc5c(c4)sc4c6ccccc6ccc54)cc3)cc2)c1] | 44.9 | 70 | 99.1 |
| J-31 | | 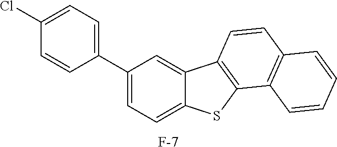 | 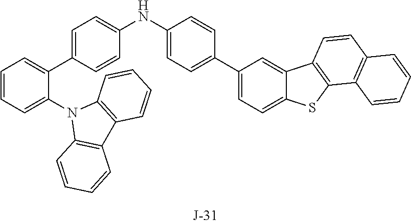 [SMILES: c1ccc(-n2c3ccccc3c3ccccc32)c(-c2ccc(Nc3ccc(-c4ccc5sc6c7ccccc7ccc6c5c4)cc3)cc2)c1] | 43.6 | 68 | 99.3 |
| J-32 | | 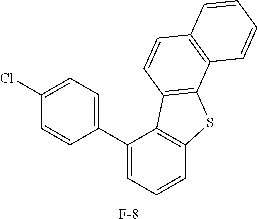 | 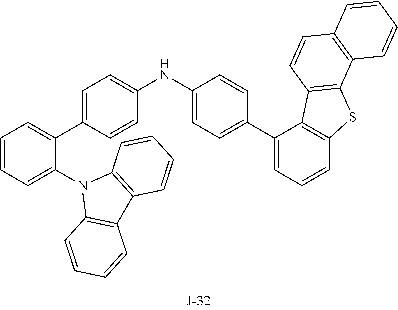 [SMILES: c1ccc(-n2c3ccccc3c3ccccc32)c(-c2ccc(Nc3ccc(-c4cccc5sc6c7ccccc7ccc6c45)cc3)cc2)c1] | 37.8 | 59 | 99.4 |
| J-33 | 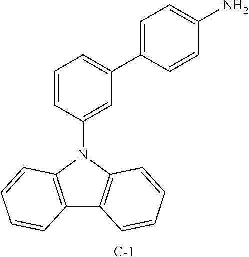 | 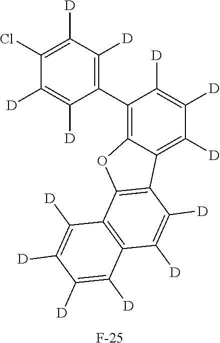 | 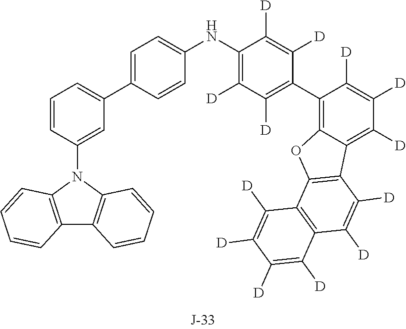 [SMILES: [2H]c1c([2H])c(-c2c([2H])c([2H])c([2H])c3c2oc2c4c([2H])c([2H])c([2H])c([2H])c4c([2H])c([2H])c23)c([2H])c([2H])c1Nc1ccc(-c2cccc(-n3c4ccccc4c4ccccc43)c2)cc1] | 43.5 | 68 | 98.5 |
| J-34 | | 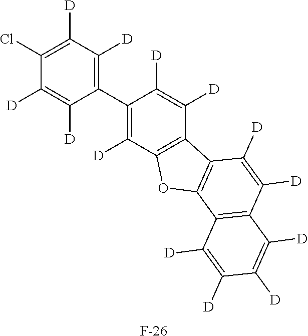 | 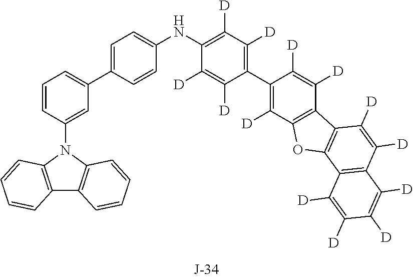 [SMILES: [2H]c1c([2H])c(-c2c([2H])c([2H])c3c(oc4c5c([2H])c([2H])c([2H])c([2H])c5c([2H])c([2H])c43)c2[2H])c([2H])c([2H])c1Nc1ccc(-c2cccc(-n3c4ccccc4c4ccccc43)c2)cc1] | 37.7 | 59 | 98.7 |
| J-35 | | 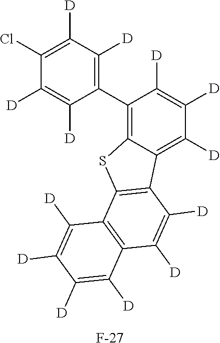 | 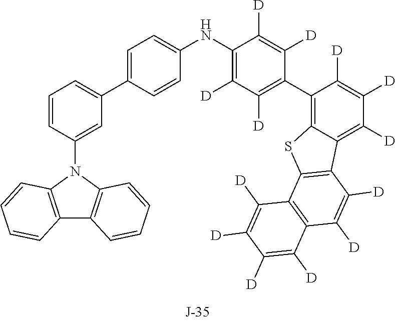 [SMILES: [2H]c1c([2H])c(-c2c([2H])c([2H])c([2H])c3c2sc2c4c([2H])c([2H])c([2H])c([2H])c4c([2H])c([2H])c23)c([2H])c([2H])c1Nc1ccc(-c2cccc(-n3c4ccccc4c4ccccc43)c2)cc1] | 45.9 | 70 | 99.0 |
| J-36 | 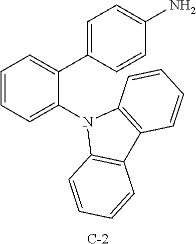 | 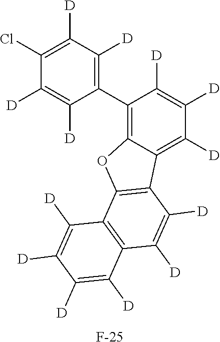 | 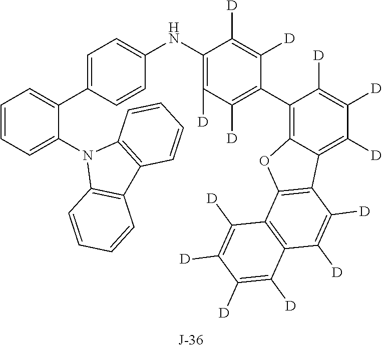 [SMILES: [2H]c1c([2H])c(-c2c([2H])c([2H])c([2H])c3c2oc2c4c([2H])c([2H])c([2H])c([2H])c4c([2H])c([2H])c23)c([2H])c([2H])c1Nc1ccc(-c2ccccc2-n2c3ccccc3c3ccccc32)cc1] | 40.9 | 64 | 97.8 |
| J-37 | | 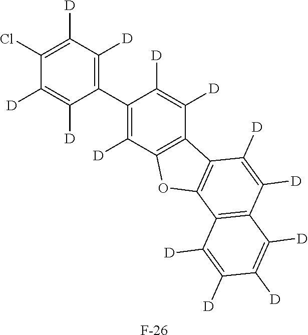 | 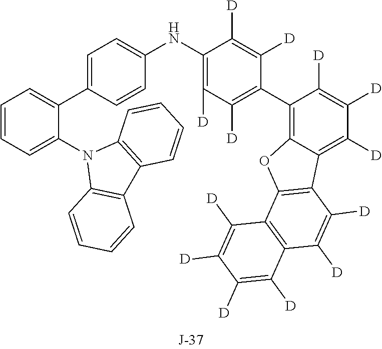 [SMILES: [2H]c1c([2H])c(-c2c([2H])c([2H])c([2H])c3c2oc2c4c([2H])c([2H])c([2H])c([2H])c4c([2H])c([2H])c23)c([2H])c([2H])c1Nc1ccc(-c2ccccc2-n2c3ccccc3c3ccccc32)cc1] | 44.1 | 69 | 98.4 |
| J-38 | | 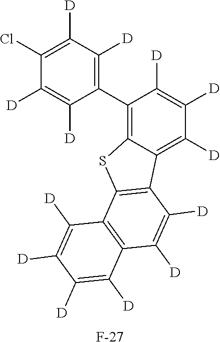 | 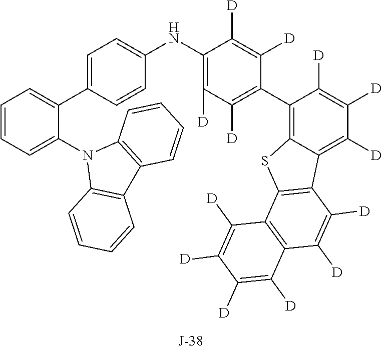 [SMILES: [2H]c1c([2H])c(-c2c([2H])c([2H])c([2H])c3c2sc2c4c([2H])c([2H])c([2H])c([2H])c4c([2H])c([2H])c23)c([2H])c([2H])c1Nc1ccc(-c2ccccc2-n2c3ccccc3c3ccccc32)cc1] | 38.7 | 59 | 98.9 |

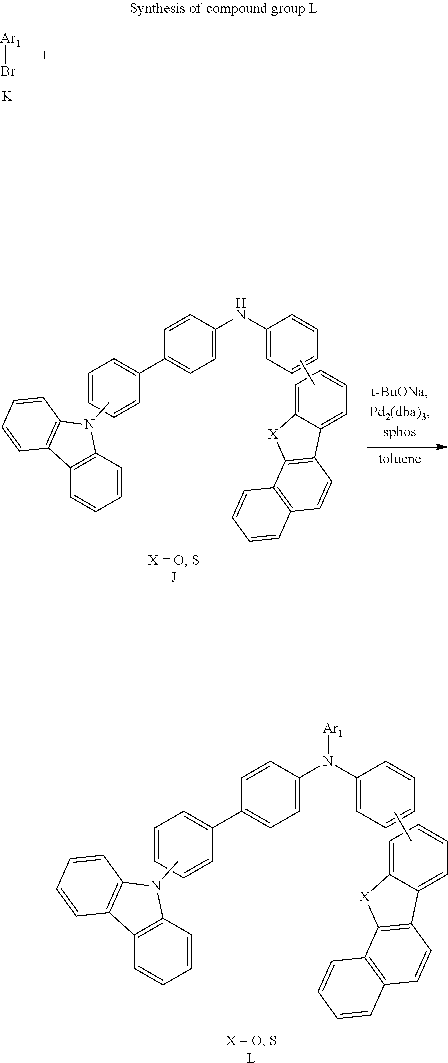

Reactant K (11 mmol), intermediate J (10 mmol), Pd₂(dba)₃ (0.183 g, 0.2 mmol), t-BuONa (1.92 g, 20 mmol), SPhos (0.164 g, 0.4 mmol), and toluene (60 mL) were added to a round bottom flask under nitrogen atmosphere and then stirred at 100° C. to perform the reaction. When the reaction was finished, the mixture was precipitated in methanol and after the precipitate was filtered, purified in a silica gel column and recrystallization method to manufacture compound group L.

The result is expressed in Table 6 below.

TABLE 6

| | | | | Ac- | | | |
|---|---|---|---|---|---|---|---|
| | | | | quired | | | MS |
| | | Intermediate group | | amount | Yield | Purity | [M + |
| Items | Reactant K | J | Compound group L | (g) | (%) | (%) | H]+ |
| Com- pound 1-1 | 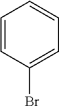 | 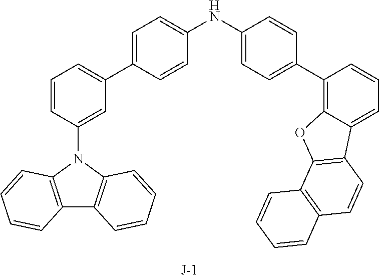 [SMILES: c1cc(-c2ccc(Nc3ccc(-c4cccc5c4oc4c6ccccc6ccc54)cc3)cc2)cc(-n2c3ccccc3c3ccccc32)c1] | 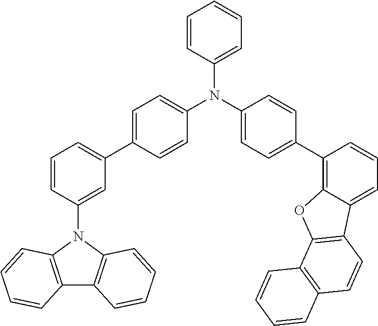 [SMILES: c1ccc(N(c2ccc(-c3cccc(-n4c5ccccc5c5ccccc54)c3)cc2)c2ccc(-c3cccc4c3oc3c5ccccc5ccc43)cc2)cc1] | 3.65 | 52 | 99.95 | 703.27 |
| Com- pound 1-121 | 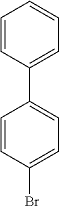 [SMILES: Brc1ccc(-c2ccccc2)cc1] | | 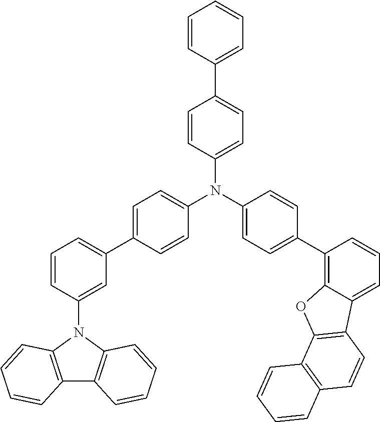 [SMILES: c1ccc(-c2ccc(N(c3ccc(-c4cccc(-n5c6ccccc6c6ccccc65)c4)cc3)c3ccc(-c4cccc5c4oc4c6ccccc6ccc54)cc3)cc2)cc1] | 3.89 | 50 | 99.93 | 779.30 |
| Com- pound 1-152 | 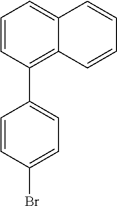 [SMILES: Brc1ccc(-c2cccc3ccccc23)cc1] | | 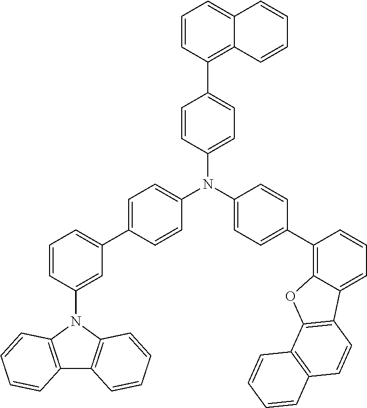 [SMILES: c1cc(-c2ccc(N(c3ccc(-c4cccc5ccccc45)cc3)c3ccc(-c4cccc5c4oc4c6ccccc6ccc54)cc3)cc2)cc(-n2c3ccccc3c3ccccc32)c1] | 3.98 | 48 | 99.92 | 829.32 |
| Com- pound 1-153 | 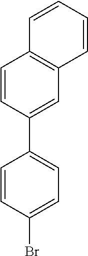 [SMILES: Brc1ccc(-c2ccc3ccccc3c2)cc1] | | 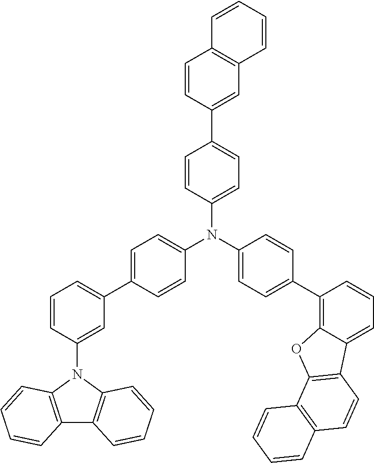 [SMILES: c1cc(-c2ccc(N(c3ccc(-c4ccc5ccccc5c4)cc3)c3ccc(-c4cccc5c4oc4c6ccccc6ccc54)cc3)cc2)cc(-n2c3ccccc3c3ccccc32)c1] | 4.64 | 56 | 99.95 | 829.33 |
| Com- pound 1-160 | 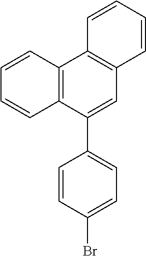 [SMILES: Brc1ccc(-c2cc3ccccc3c3ccccc23)cc1] | | 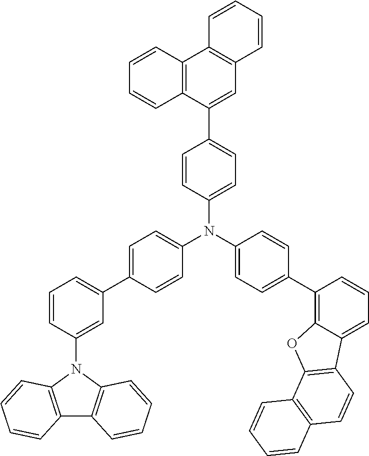 [SMILES: c1cc(-c2ccc(N(c3ccc(-c4cc5ccccc5c5ccccc45)cc3)c3ccc(-c4cccc5c4oc4c6ccccc6ccc54)cc3)cc2)cc(-n2c3ccccc3c3ccccc32)c1] | 5.27 | 60 | 99.90 | 879.33 |
| Com- pound 1-313 | 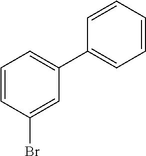 [SMILES: Brc1cccc(-c2ccccc2)c1] | | 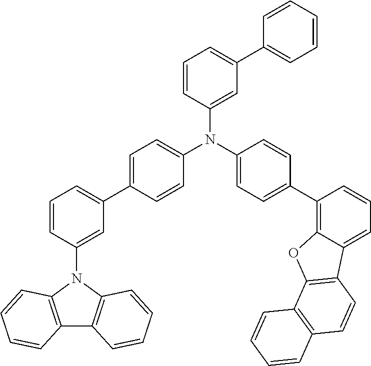 [SMILES: c1ccc(-c2cccc(N(c3ccc(-c4cccc(-n5c6ccccc6c6ccccc65)c4)cc3)c3ccc(-c4cccc5c4oc4c6ccccc6ccc54)cc3)c2)cc1] | 4.12 | 53 | 99.96 | 779.30 |
| Com- pound 1-314 | 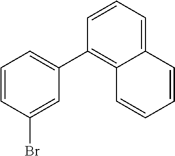 [SMILES: Brc1cccc(-c2cccc3ccccc23)c1] | | 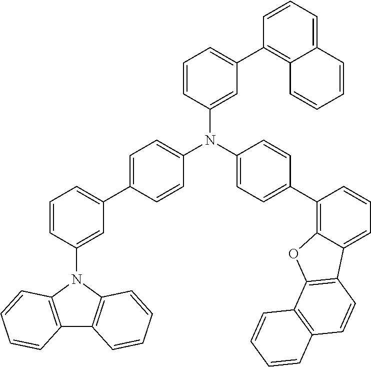 [SMILES: c1cc(-c2cccc3ccccc23)cc(N(c2ccc(-c3cccc(-n4c5ccccc5c5ccccc54)c3)cc2)c2ccc(-c3cccc4c3oc3c5ccccc5ccc43)cc2)c1] | 4.64 | 56 | 99.95 | 829.32 |
| Com- pound 1-321 | 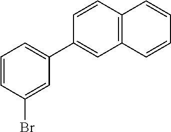 [SMILES: Brc1cccc(-c2ccc3ccccc3c2)c1] | | 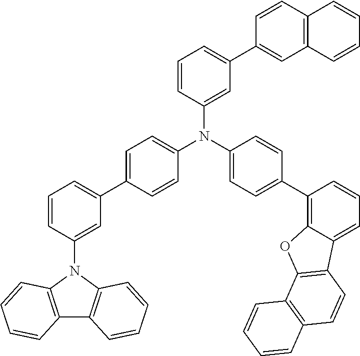 [SMILES: c1cc(-c2ccc3ccccc3c2)cc(N(c2ccc(-c3cccc(-n4c5ccccc5c5ccccc54)c3)cc2)c2ccc(-c3cccc4c3oc3c5ccccc5ccc43)cc2)c1] | 4.47 | 54 | 99.90 | 829.32 |
| Com- pound 1-217 | 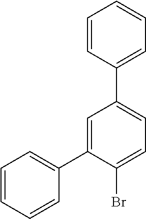 [SMILES: Brc1ccc(-c2ccccc2)cc1-c1ccccc1] | | 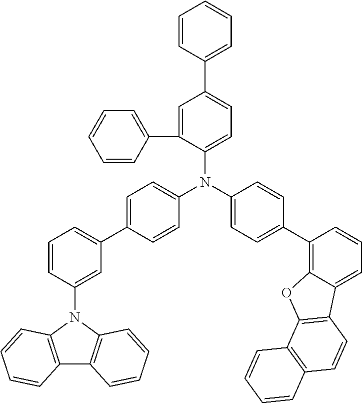 [SMILES: c1ccc(-c2ccc(N(c3ccc(-c4cccc(-n5c6ccccc6c6ccccc65)c4)cc3)c3ccc(-c4cccc5c4oc4c6ccccc6ccc54)cc3)c(-c3ccccc3)c2)cc1] | 4.19 | 49 | 99.94 | 855.33 |
| Com- pound 1-409 | 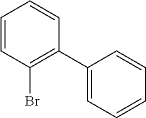 [SMILES: Brc1ccccc1-c1ccccc1] | | 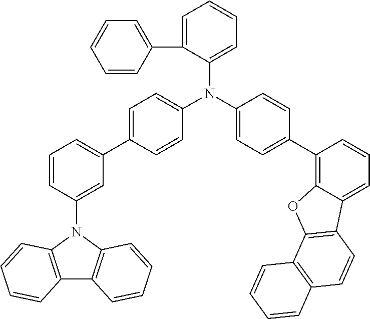 [SMILES: c1ccc(-c2ccccc2N(c2ccc(-c3cccc(-n4c5ccccc5c5ccccc54)c3)cc2)c2ccc(-c3cccc4c3oc3c5ccccc5ccc43)cc2)cc1] | 4.36 | 56 | 99.95 | 779.31 |
| Com- pound 1-285 | 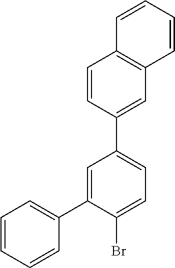 [SMILES: Brc1ccc(-c2ccc3ccccc3c2)cc1-c1ccccc1] | | 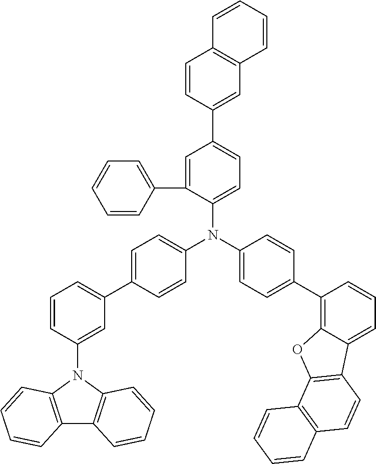 [SMILES: c1ccc(-c2cc(-c3ccc4ccccc4c3)ccc2N(c2ccc(-c3cccc(-n4c5ccccc5c5ccccc54)c3)cc2)c2ccc(-c3cccc4c3oc3c5ccccc5ccc43)cc2)cc1] | 5.25 | 58 | 99.96 | 905.35 |
| Com- pound 1-505 | 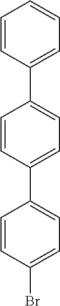 [SMILES: Brc1ccc(-c2ccc(-c3ccccc3)cc2)cc1] | | 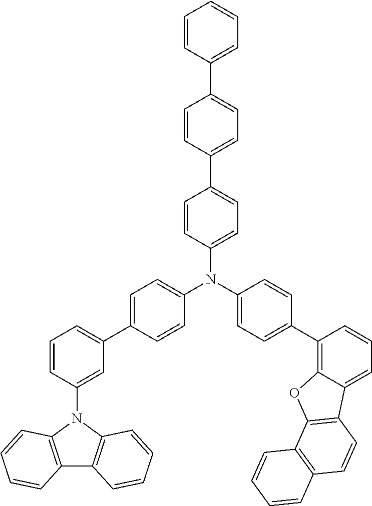 [SMILES: c1ccc(-c2ccc(-c3ccc(N(c4ccc(-c5cccc(-n6c7ccccc7c7ccccc76)c5)cc4)c4ccc(-c5cccc6c5oc5c7ccccc7ccc65)cc4)cc3)cc2)cc1] | 4.87 | 57 | 99.98 | 905.36 |
| Com- pound 1-701 | 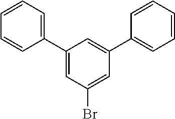 [SMILES: Brc1cc(-c2ccccc2)cc(-c2ccccc2)c1] | | 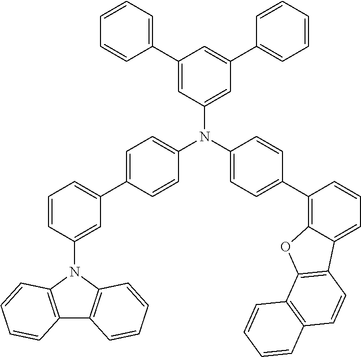 [SMILES: c1ccc(-c2cc(-c3ccccc3)cc(N(c3ccc(-c4cccc(-n5c6ccccc6c6ccccc65)c4)cc3)c3ccc(-c4cccc5c4oc4c6ccccc6ccc54)cc3)c2)cc1] | 4.78 | 56 | 99.92 | 905.35 |
| Com- pound 1-702 | 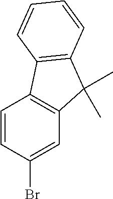 [SMILES: CC1(C)c2ccccc2-c2ccc(Br)cc21] | | 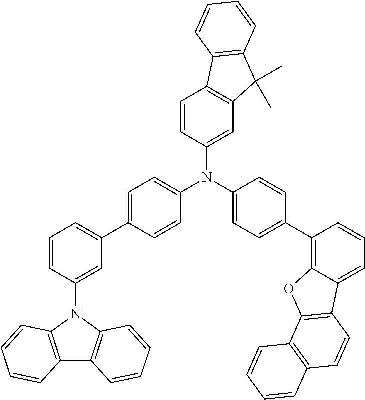 [SMILES: CC1(C)c2ccccc2-c2ccc(N(c3ccc(-c4cccc(-n5c6ccccc6c6ccccc65)c4)cc3)c3ccc(-c4cccc5c4oc4c6ccccc6ccc54)cc3)cc21] | 4.66 | 57 | 99.92 | 819.33 |
| Com- pound 1-703 | 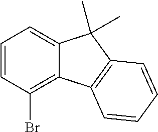 | | 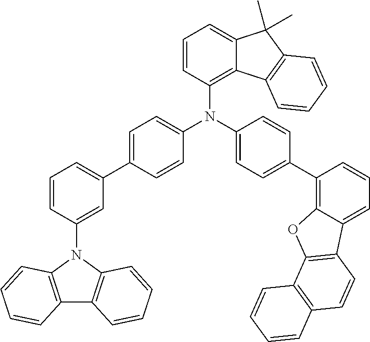 [SMILES: CC1(C)c2ccccc2-c2c(N(c3ccc(-c4cccc(-n5c6ccccc6c6ccccc65)c4)cc3)c3ccc(-c4cccc5c4oc4c6ccccc6ccc54)cc3)cccc21] | 5.24 | 64 | 99.90 | 819.33 |
| Com- pound 1-704 | 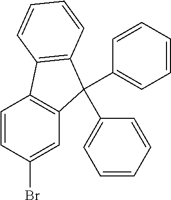 [SMILES: Brc1ccc2c(c1)C(c1ccccc1)(c1ccccc1)c1ccccc1-2] | | 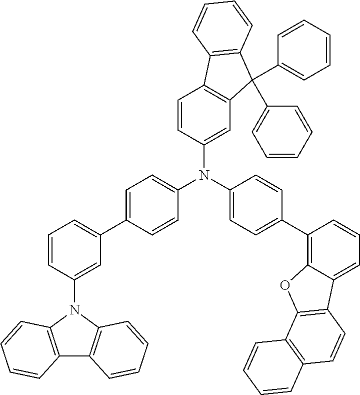 [SMILES: c1ccc(C2(c3ccccc3)c3ccccc3-c3ccc(N(c4ccc(-c5cccc(-n6c7ccccc7c7ccccc76)c5)cc4)c4ccc(-c5cccc6c5oc5c7ccccc7ccc65)cc4)cc32)cc1] | 5.47 | 58 | 99.97 | 943.36 |
| Com- pound 1-705 | 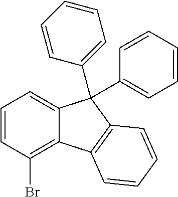 [SMILES: Brc1cccc2c1-c1ccccc1C2(c1ccccc1)c1ccccc1] | | 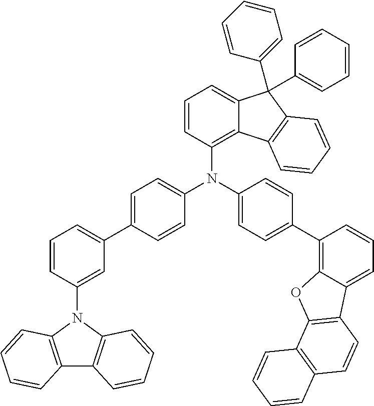 [SMILES: c1ccc(C2(c3ccccc3)c3ccccc3-c3c(N(c4ccc(-c5cccc(-n6c7ccccc7c7ccccc76)c5)cc4)c4ccc(-c5cccc6c5oc5c7ccccc7ccc65)cc4)cccc32)cc1] | 5.65 | 60 | 99.98 | 943.36 |
| Com- pound 1-706 | 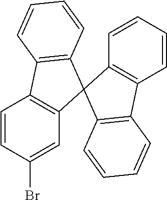 [SMILES: Brc1ccc2c(c1)C1(c3ccccc3-c3ccccc31)c1ccccc1-2] | | 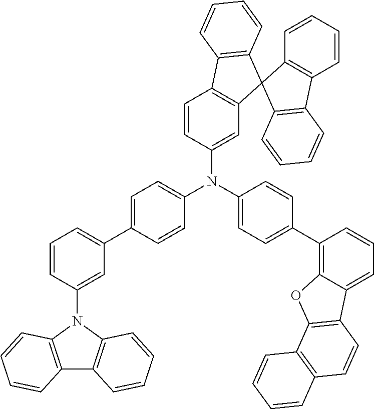 [SMILES: c1cc(-c2ccc(N(c3ccc(-c4cccc5c4oc4c6ccccc6ccc54)cc3)c3ccc4c(c3)C3(c5ccccc5-c5ccccc53)c3ccccc3-4)cc2)cc(-n2c3ccccc3c3ccccc32)c1] | 5.56 | 59 | 99.90 | 941.35 |
| Com- pound 1-707 | 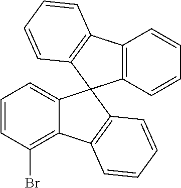 [SMILES: Brc1cccc2c1-c1ccccc1C21c2ccccc2-c2ccccc21] | | 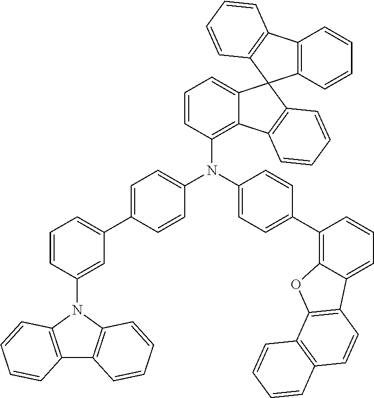 [SMILES: c1cc(-c2ccc(N(c3ccc(-c4cccc5c4oc4c6ccccc6ccc54)cc3)c3cccc4c3-c3ccccc3C43c4ccccc4-c4ccccc43)cc2)cc(-n2c3ccccc3c3ccccc32)c1] | 5.75 | 61 | 99.92 | 941.35 |
| Com- pound 1-981 | 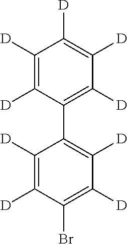 [SMILES: [2H]c1c([2H])c([2H])c(-c2c([2H])c([2H])c(Br)c([2H])c2[2H])c([2H])c1[2H]] | | 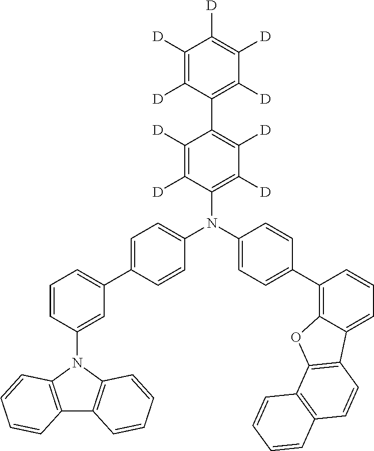 [SMILES: [2H]c1c([2H])c([2H])c(-c2c([2H])c([2H])c(N(c3ccc(-c4cccc(-n5c6ccccc6c6ccccc65)c4)cc3)c3ccc(-c4cccc5c4oc4c6ccccc6ccc54)cc3)c([2H])c2[2H])c([2H])c1[2H]] | 5.35 | 68 | 99.94 | 788.36 |
| Com- pound 1-982 | 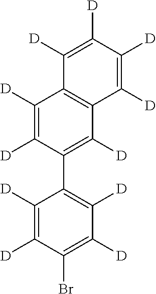 [SMILES: [2H]c1c([2H])c(-c2c([2H])c([2H])c3c([2H])c([2H])c([2H])c([2H])c3c2[2H])c([2H])c([2H])c1Br] | | 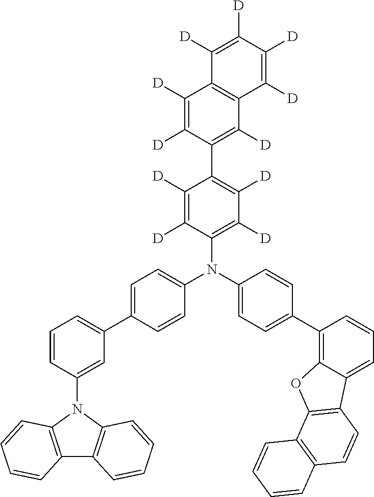 [SMILES: [2H]c1c([2H])c(N(c2ccc(-c3cccc(-n4c5ccccc5c5ccccc54)c3)cc2)c2ccc(-c3cccc4c3oc3c5ccccc5ccc43)cc2)c([2H])c([2H])c1-c1c([2H])c([2H])c2c([2H])c([2H])c([2H])c([2H])c2c1[2H]] | 4.20 | 50 | 99.94 | 840.39 |
| Com- pound 1-31 | 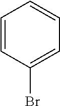 | 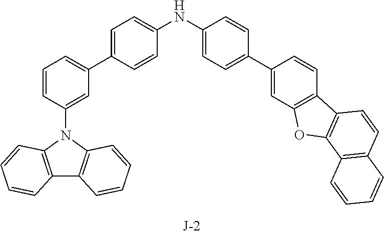 [SMILES: c1cc(-c2ccc(Nc3ccc(-c4ccc5c(c4)oc4c6ccccc6ccc54)cc3)cc2)cc(-n2c3ccccc3c3ccccc32)c1] | 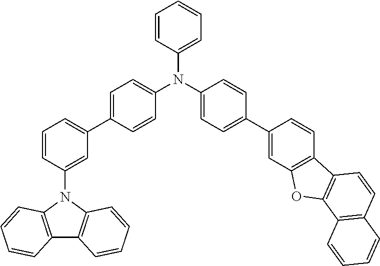 [SMILES: c1ccc(N(c2ccc(-c3cccc(-n4c5ccccc5c5ccccc54)c3)cc2)c2ccc(-c3ccc4c(c3)oc3c5ccccc5ccc43)cc2)cc1] | 4.07 | 58 | 99.92 | 703.27 |
| Com- pound 1-127 | 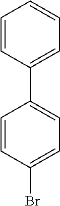 [SMILES: Brc1ccc(-c2ccccc2)cc1] | | 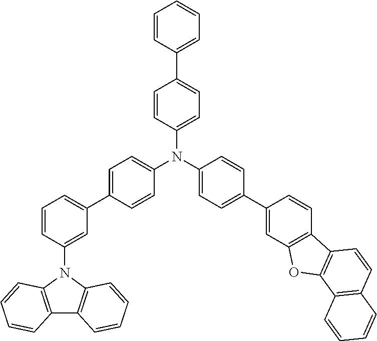 [SMILES: c1ccc(-c2ccc(N(c3ccc(-c4cccc(-n5c6ccccc6c6ccccc65)c4)cc3)c3ccc(-c4ccc5c(c4)oc4c6ccccc6ccc54)cc3)cc2)cc1] | 4.75 | 61 | 99.94 | 779.30 |
| Com- pound 1-128 | 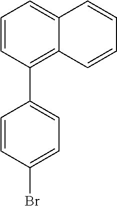 [SMILES: Brc1ccc(-c2cccc3ccccc23)cc1] | | 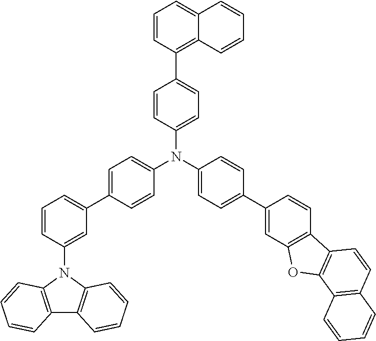 [SMILES: c1cc(-c2ccc(N(c3ccc(-c4ccc5c(c4)oc4c6ccccc6ccc54)cc3)c3ccc(-c4cccc5ccccc45)cc3)cc2)cc(-n2c3ccccc3c3ccccc32)c1] | 5.55 | 67 | 99.97 | 829.32 |
| Com- pound 1-129 | 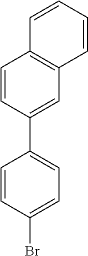 [SMILES: Brc1ccc(-c2ccc3ccccc3c2)cc1] | | 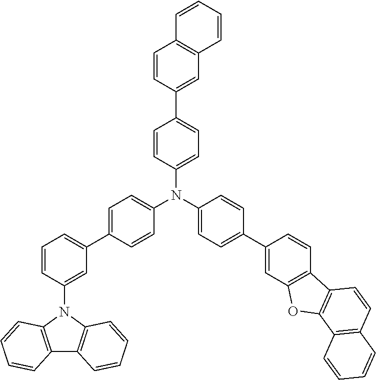 [SMILES: c1cc(-c2ccc(N(c3ccc(-c4ccc5ccccc5c4)cc3)c3ccc(-c4ccc5c(c4)oc4c6ccccc6ccc54)cc3)cc2)cc(-n2c3ccccc3c3ccccc32)c1] | 4.89 | 59 | 99.96 | 829.32 |
| Com- pound 1-130 | 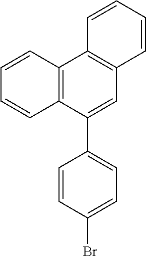 [SMILES: Brc1ccc(-c2cc3ccccc3c3ccccc23)cc1] | | 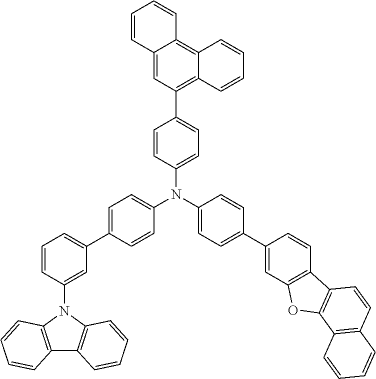 [SMILES: c1cc(-c2ccc(N(c3ccc(-c4ccc5c(c4)oc4c6ccccc6ccc54)cc3)c3ccc(-c4cc5ccccc5c5ccccc45)cc3)cc2)cc(-n2c3ccccc3c3ccccc32)c1] | 4.65 | 53 | 99.95 | 879.33 |
| Com- pound 1-319 | 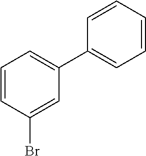 [SMILES: Brc1cccc(-c2ccccc2)c1] | | 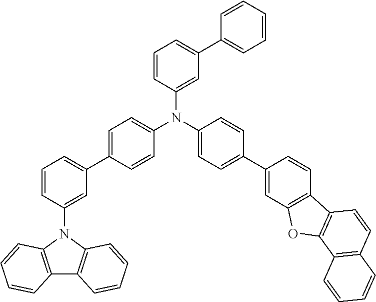 [SMILES: c1ccc(-c2cccc(N(c3ccc(-c4cccc(-n5c6ccccc6c6ccccc65)c4)cc3)c3ccc(-c4ccc5c(c4)oc4c6ccccc6ccc54)cc3)c2)cc1] | 4.83 | 62 | 99.90 | 779.30 |
| Com- pound 1-320 | 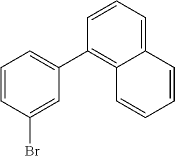 [SMILES: Brc1cccc(-c2cccc3ccccc23)c1] | | 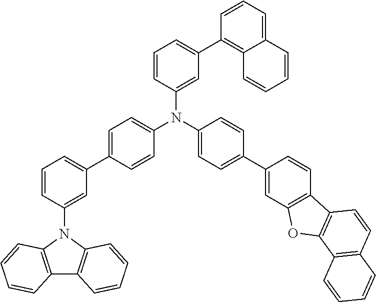 [SMILES: c1cc(-c2cccc3ccccc23)cc(N(c2ccc(-c3cccc(-n4c5ccccc5c5ccccc54)c3)cc2)c2ccc(-c3ccc4c(c3)oc3c5ccccc5ccc43)cc2)c1] | 4.97 | 60 | 99.94 | 829.31 |
| Com- pound 1-321 | 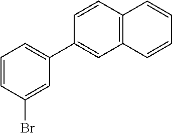 [SMILES: Brc1cccc(-c2ccc3ccccc3c2)c1] | | 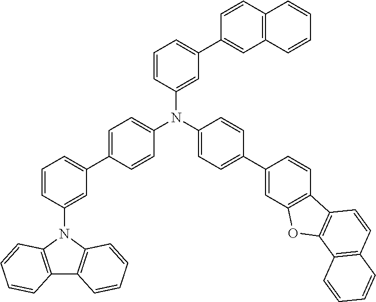 [SMILES: c1cc(-c2ccc3ccccc3c2)cc(N(c2ccc(-c3cccc(-n4c5ccccc5c5ccccc54)c3)cc2)c2ccc(-c3ccc4c(c3)oc3c5ccccc5ccc43)cc2)c1] | 5.05 | 61 | 99.95 | 829.32 |
| Com- pound 1-223 | 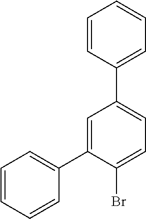 [SMILES: Brc1ccc(-c2ccccc2)cc1-c1ccccc1] | | 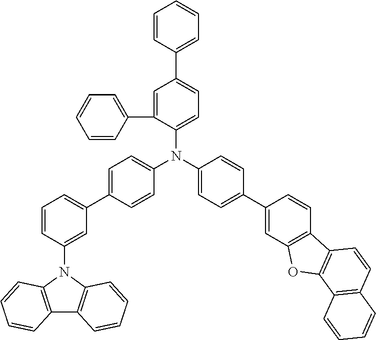 [SMILES: c1ccc(-c2ccc(N(c3ccc(-c4cccc(-n5c6ccccc6c6ccccc65)c4)cc3)c3ccc(-c4ccc5c(c4)oc4c6ccccc6ccc54)cc3)c(-c3ccccc3)c2)cc1] | 4.61 | 54 | 99.96 | 855.33 |
| Com- pound 1-415 | 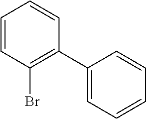 [SMILES: Brc1ccccc1-c1ccccc1] | | 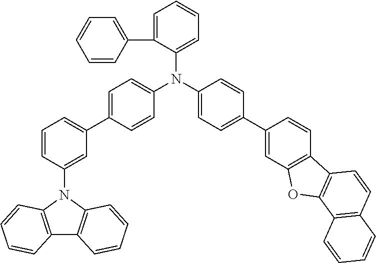 [SMILES: c1ccc(-c2ccccc2N(c2ccc(-c3cccc(-n4c5ccccc5c5ccccc54)c3)cc2)c2ccc(-c3ccc4c(c3)oc3c5ccccc5ccc43)cc2)cc1] | 4.12 | 53 | 99.90 | 779.30 |
| Com- pound 1-225 | 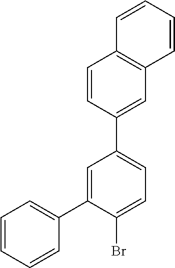 [SMILES: Brc1ccc(-c2ccc3ccccc3c2)cc1-c1ccccc1] | | 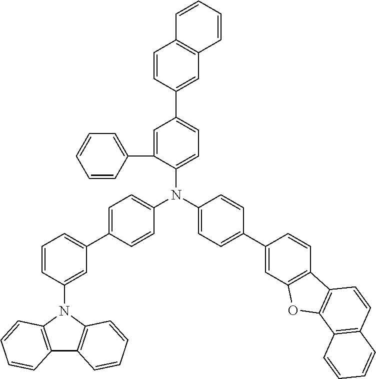 [SMILES: c1ccc(-c2cc(-c3ccc4ccccc4c3)ccc2N(c2ccc(-c3cccc(-n4c5ccccc5c5ccccc54)c3)cc2)c2ccc(-c3ccc4c(c3)oc3c5ccccc5ccc43)cc2)cc1] | 4.97 | 55 | 99.93 | 905.35 |
| Com- pound 1-487 | 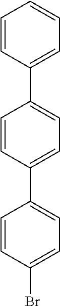 [SMILES: Brc1ccc(-c2ccc(-c3ccccc3)cc2)cc1] | | 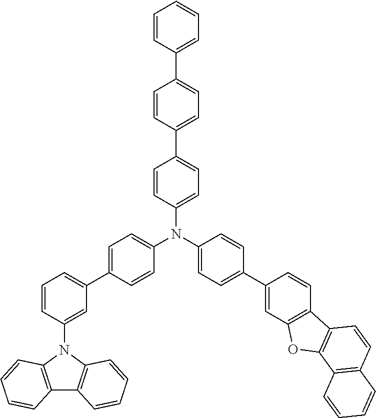 [SMILES: c1ccc(-c2ccc(-c3ccc(N(c4ccc(-c5cccc(-n6c7ccccc7c7ccccc76)c5)cc4)c4ccc(-c5ccc6c(c5)oc5c7ccccc7ccc65)cc4)cc3)cc2)cc1] | 5.13 | 60 | 99.95 | 855.32 |
| Com- pound 1-708 | 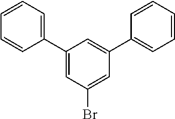 [SMILES: Brc1cc(-c2ccccc2)cc(-c2ccccc2)c1] | | 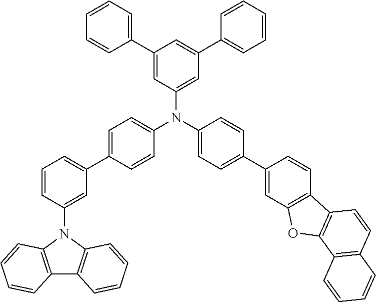 [SMILES: c1ccc(-c2cc(-c3ccccc3)cc(N(c3ccc(-c4cccc(-n5c6ccccc6c6ccccc65)c4)cc3)c3ccc(-c4ccc5c(c4)oc4c6ccccc6ccc54)cc3)c2)cc1] | 5.21 | 61 | 99.94 | 855.32 |
| Com- pound 1-709 | 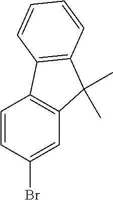 [SMILES: CC1(C)c2ccccc2-c2ccc(Br)cc21] | | 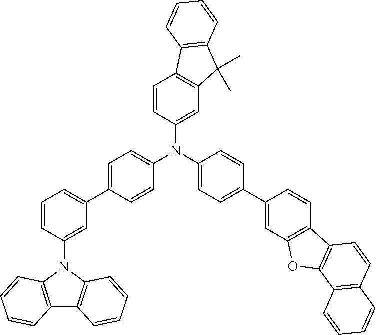 [SMILES: CC1(C)c2ccccc2-c2ccc(N(c3ccc(-c4cccc(-n5c6ccccc6c6ccccc65)c4)cc3)c3ccc(-c4ccc5c(c4)oc4c6ccccc6ccc54)cc3)cc21] | 5.32 | 65 | 99.98 | 819.33 |
| Com- pound 1-710 | 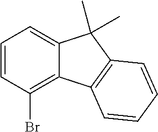 [SMILES: CC1(C)c2ccccc2-c2c(Br)cccc21] | | 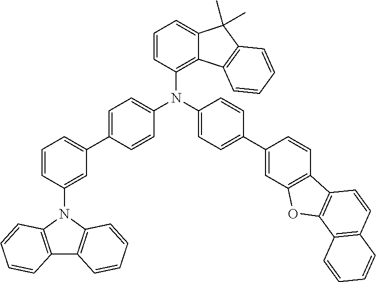 [SMILES: CC1(C)c2ccccc2-c2c(N(c3ccc(-c4cccc(-n5c6ccccc6c6ccccc65)c4)cc3)c3ccc(-c4ccc5c(c4)oc4c6ccccc6ccc54)cc3)cccc21] | 5.24 | 64 | 99.97 | 819.33 |
| Com- pound 1-711 | 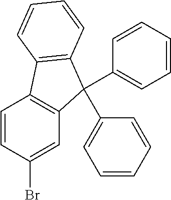 [SMILES: Brc1ccc2c(c1)C(c1ccccc1)(c1ccccc1)c1ccccc1-2] | | 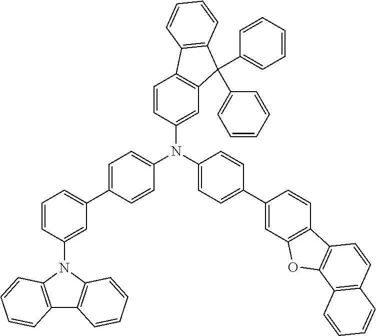 [SMILES: c1ccc(C2(c3ccccc3)c3ccccc3-c3ccc(N(c4ccc(-c5cccc(-n6c7ccccc7c7ccccc76)c5)cc4)c4ccc(-c5ccc6c(c5)oc5c7ccccc7ccc65)cc4)cc32)cc1] | 6.03 | 64 | 99.96 | 943.36 |
| Com- pound 1-712 | 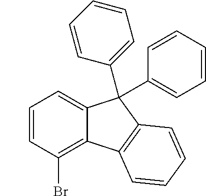 | | 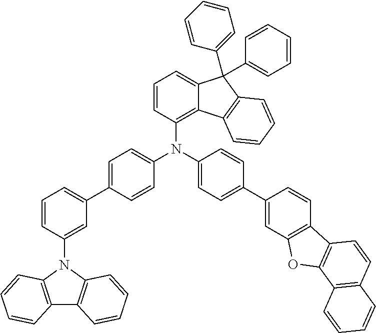 [SMILES: c1ccc(C2(c3ccccc3)c3ccccc3-c3c(N(c4ccc(-c5cccc(-n6c7ccccc7c7ccccc76)c5)cc4)c4ccc(-c5ccc6c(c5)oc5c7ccccc7ccc65)cc4)cccc32)cc1] | 5.65 | 60 | 99.95 | 943.37 |
| Com- pound 1-713 | 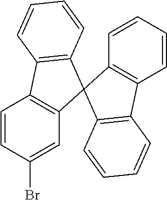 [SMILES: Brc1ccc2c(c1)C1(c3ccccc3-c3ccccc31)c1ccccc1-2] | | 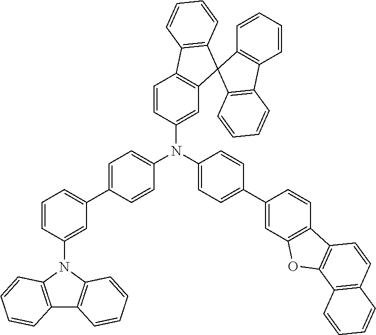 [SMILES: c1cc(-c2ccc(N(c3ccc(-c4ccc5c(c4)oc4c6ccccc6ccc54)cc3)c3ccc4c(c3)C3(c5ccccc5-c5ccccc53)c3ccccc3-4)cc2)cc(-n2c3ccccc3c3ccccc32)c1] | 5.39 | 58 | 99.93 | 941.35 |
| Com- pound 1-714 | 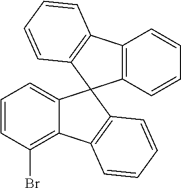 | | 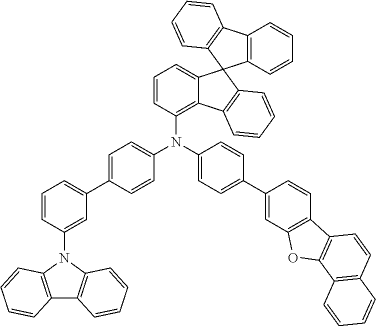 [SMILES: c1cc(-c2ccc(N(c3ccc(-c4ccc5c(c4)oc4c6ccccc6ccc54)cc3)c3cccc4c3-c3ccccc3C43c4ccccc4-c4ccccc43)cc2)cc(-n2c3ccccc3c3ccccc32)c1] | 5.09 | 54 | 99.91 | 941.35 |
| Com- pound 1-983 | 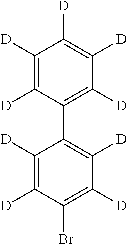 [SMILES: [2H]c1c([2H])c([2H])c(-c2c([2H])c([2H])c(Br)c([2H])c2[2H])c([2H])c1[2H]] | | 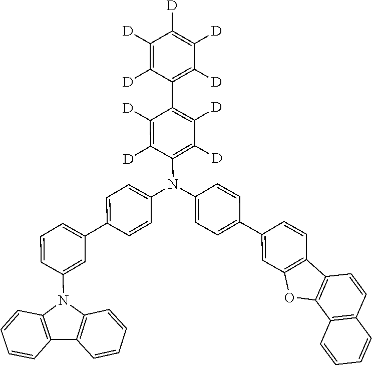 | 4.65 | 59 | 99.92 | 788.36 |
| Com- pound 1-984 | 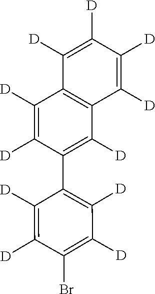 [SMILES: [2H]c1c([2H])c(-c2c([2H])c([2H])c3c([2H])c([2H])c([2H])c([2H])c3c2[2H])c([2H])c([2H])c1Br] | | 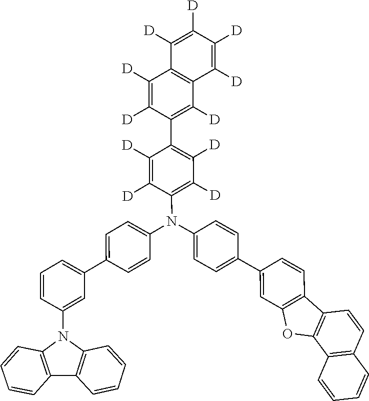 | 5.04 | 60 | 99.96 | 840.39 |
| Com- pound 1-135 | 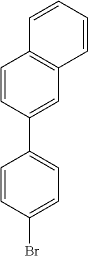 [SMILES: Brc1ccc(-c2ccc3ccccc3c2)cc1] | 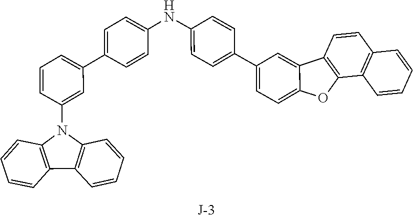 | 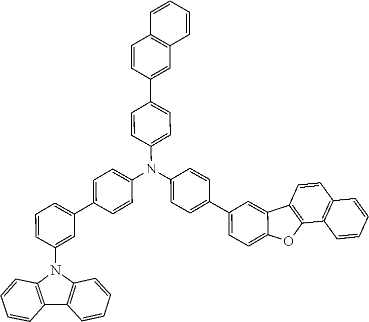 | 5.63 | 68 | 99.95 | 829.32 |
| Com- pound 1-301 | 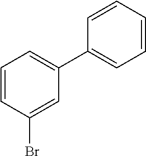 [SMILES: Brc1cccc(-c2ccccc2)c1] | | 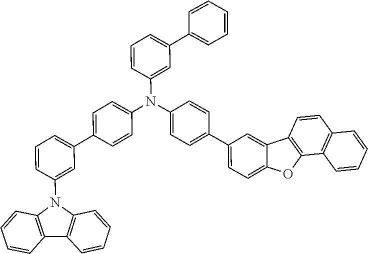 | 4.51 | 58 | 99.90 | 829.32 |
| Com- pound 1-229 | 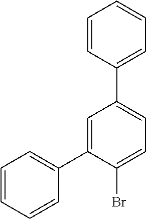 [SMILES: Brc1ccc(-c2ccccc2)cc1-c1ccccc1] | | 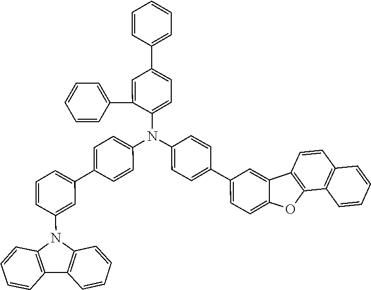 | 4.70 | 55 | 99.95 | 855.33 |
| Com- pound 1-517 | 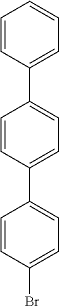 [SMILES: Brc1ccc(-c2ccc(-c3ccccc3)cc2)cc1] | | 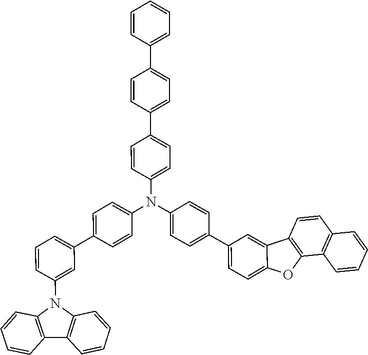 | 4.61 | 54 | 99.94 | 855.32 |
| Com- pound 1-706 | 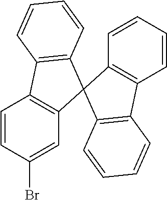 [SMILES: Brc1ccc2c(c1)C1(c3ccccc3-c3ccccc31)c1ccccc1-2] | | 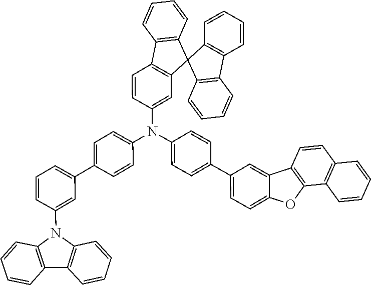 | 5.56 | 59 | 99.95 | 941.35 |
| Com- pound 1-707 | 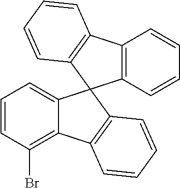 [SMILES: Brc1cccc2c1-c1ccccc1C21c2ccccc2-c2ccccc21] | | 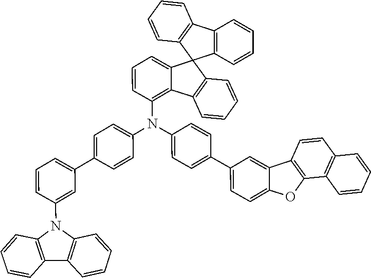 | 5.47 | 58 | 99.96 | 941.35 |
| Com- pound 1-985 | 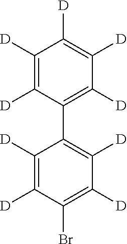 [SMILES: [2H]c1c([2H])c([2H])c(-c2c([2H])c([2H])c(Br)c([2H])c2[2H])c([2H])c1[2H]] | | 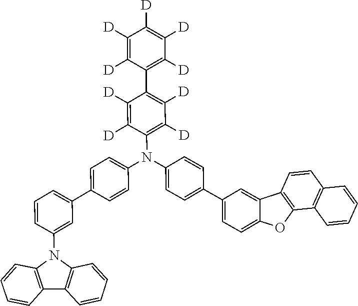 | 4.09 | 52 | 99.97 | 788.35 |
| Com- pound 1-986 | 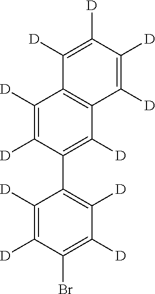 [SMILES: [2H]c1c([2H])c(-c2c([2H])c([2H])c3c([2H])c([2H])c([2H])c([2H])c3c2[2H])c([2H])c([2H])c1Br] | | 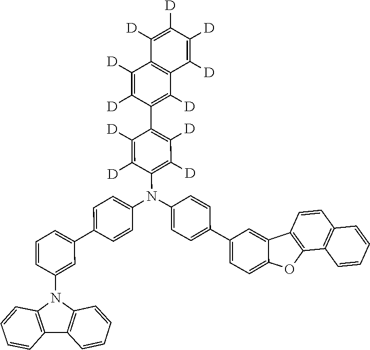 | 5.04 | 60 | 99.91 | 840.39 |
| Com- pound 1-414 | 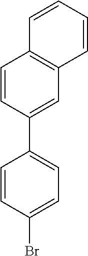 [SMILES: Brc1ccc(-c2ccc3ccccc3c2)cc1] | 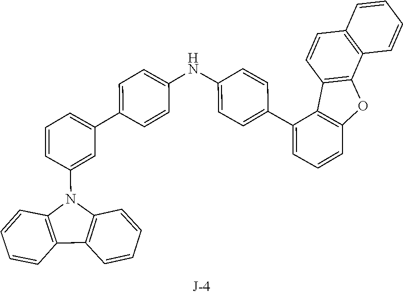 | 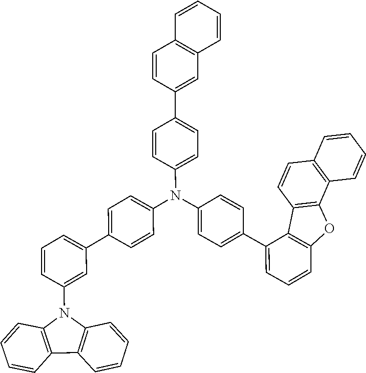 | 5.05 | 61 | 99.94 | 829.32 |
| Com- pound 1-727 | 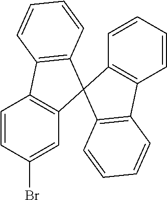 [SMILES: Brc1ccc2c(c1)C1(c3ccccc3-c3ccccc31)c1ccccc1-2] | | 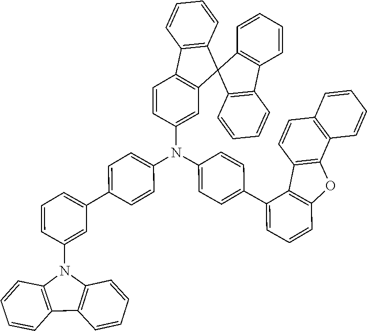 | 6.03 | 64 | 99.93 | 941.35 |
| Com- pound 1-728 | 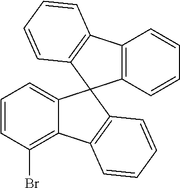 [SMILES: Brc1cccc2c1-c1ccccc1C21c2ccccc2-c2ccccc21] | | 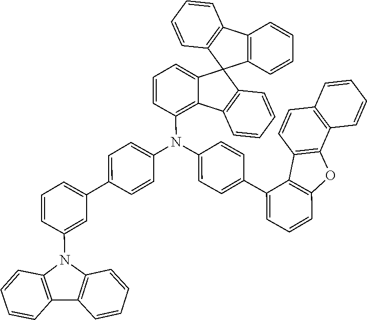 | 5.56 | 59 | 99.90 | 941.35 |
| Com- pound 1-797 | 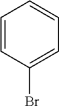 | 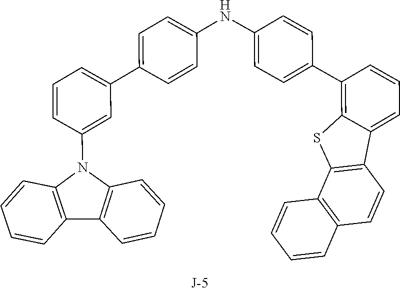 | 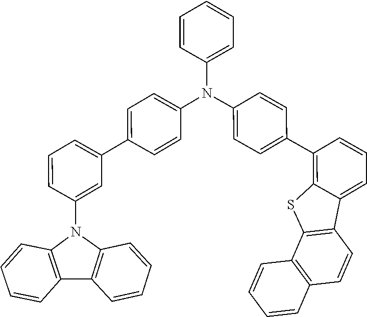 | 3.52 | 49 | 99.94 | 719.25 |
| Com- pound 1-821 | 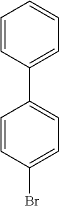 [SMILES: Brc1ccc(-c2ccccc2)cc1] | | 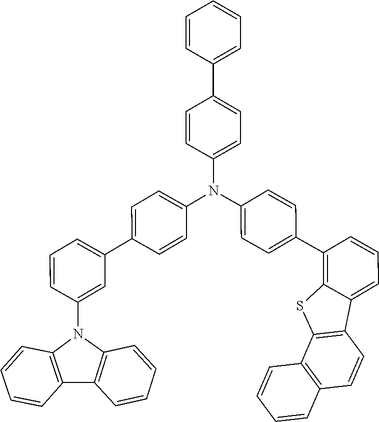 | 4.61 | 58 | 99.95 | 795.28 |
| Com- pound 1-822 | 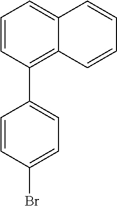 [SMILES: Brc1ccc(-c2cccc3ccccc23)cc1] | | 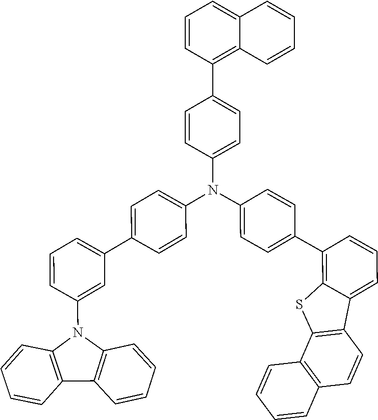 | 4.73 | 56 | 99.91 | 845.29 |
| Com- pound 1-823 | 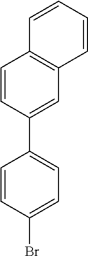 [SMILES: Brc1ccc(-c2ccc3ccccc3c2)cc1] | | 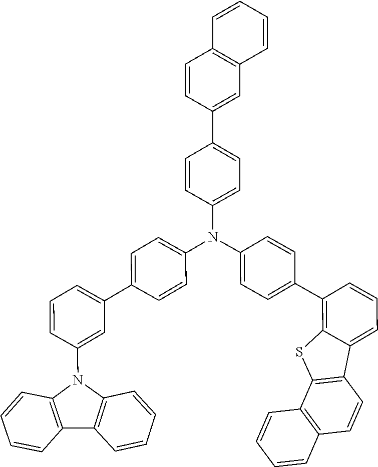 | 4.56 | 54 | 99.96 | 845.29 |
| Com- pound 1-824 | 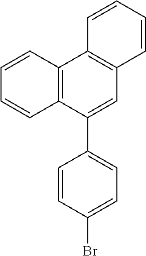 [SMILES: Brc1ccc(-c2cc3ccccc3c3ccccc23)cc1] | | 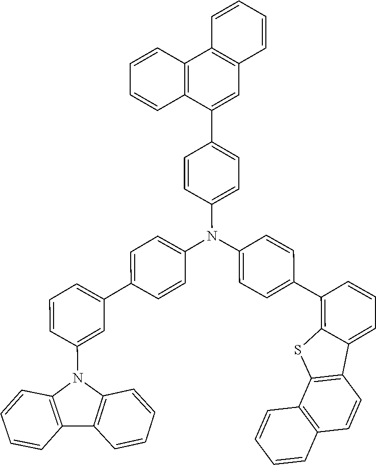 | 5.81 | 65 | 99.92 | 895.31 |
| Com- pound 1-869 | 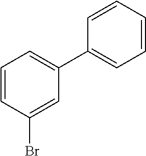 [SMILES: Brc1cccc(-c2ccccc2)c1] | | 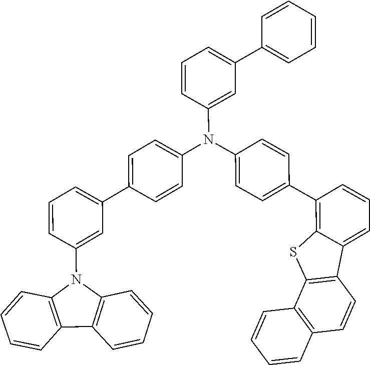 | 4.85 | 61 | 99.93 | 795.28 |
| Com- pound 1-864 | 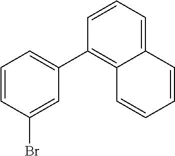 [SMILES: Brc1cccc(-c2cccc3ccccc23)c1] | | 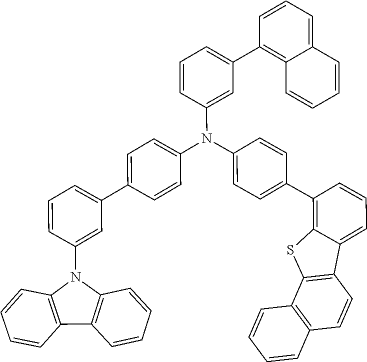 | 4.30 | 51 | 99.95 | 845.29 |
| Com- pound 1-865 | 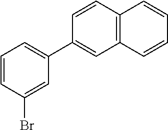 [SMILES: Brc1cccc(-c2ccc3ccccc3c2)c1] | | 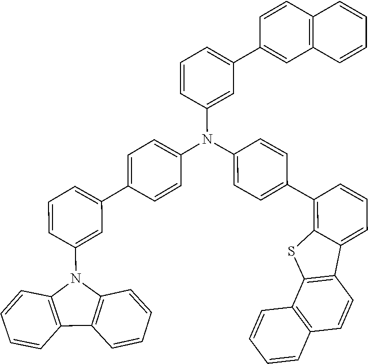 | 4.47 | 53 | 99.96 | 845.30 |
| Com- pound 1-845 | 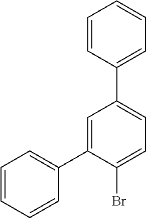 [SMILES: Brc1ccc(-c2ccccc2)cc1-c1ccccc1] | | 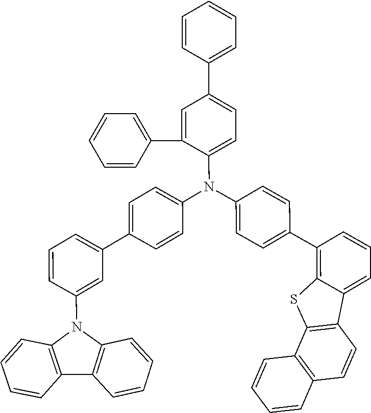 | 5.05 | 58 | 99.94 | 871.31 |
| Com- pound 1-893 | 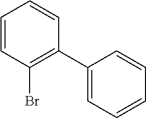 [SMILES: Brc1ccccc1-c1ccccc1] | | 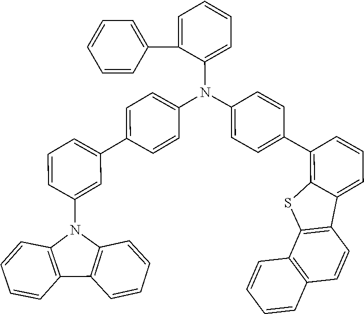 | 4.37 | 55 | 99.96 | 795.28 |
| Com- pound 1-847 | 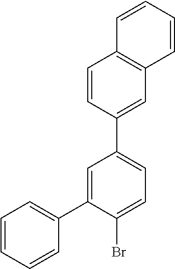 [SMILES: Brc1ccc(-c2ccc3ccccc3c2)cc1-c1ccccc1] | | 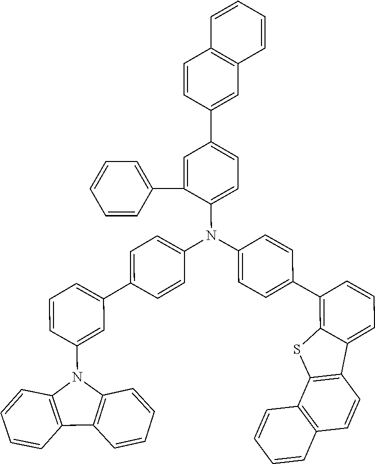 | 5.15 | 56 | 99.98 | 921.33 |
| Com- pound 1-917 | 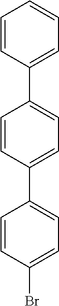 [SMILES: Brc1ccc(-c2ccc(-c3ccccc3)cc2)cc1] | | 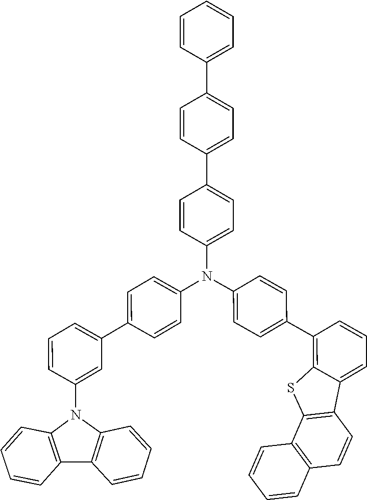 | 4.87 | 56 | 99.95 | 871.31 |
| Com- pound 1-967 | 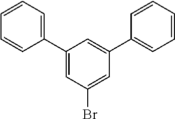 [SMILES: Brc1cc(-c2ccccc2)cc(-c2ccccc2)c1] | | 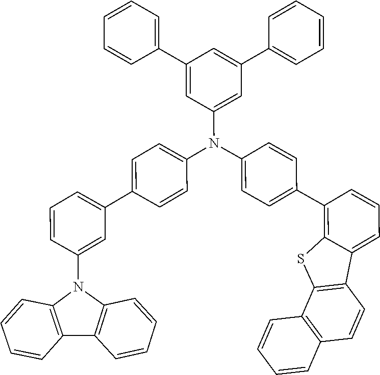 | 4.52 | 52 | 99.91 | 871.32 |
| Com- pound 1-968 | 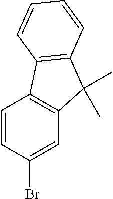 [SMILES: CC1(C)c2ccccc2-c2ccc(Br)cc21] | | 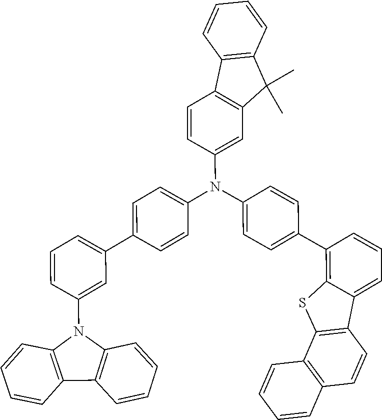 | 4.17 | 50 | 99.98 | 835.31 |
| Com- pound 1-969 | 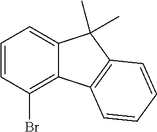 [SMILES: CC1(C)c2ccccc2-c2c(Br)cccc21] | | 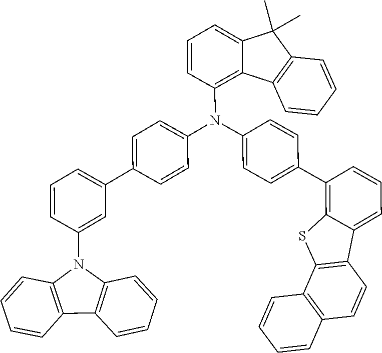 | 4.50 | 54 | 99.97 | 835.31 |
| Com- pound 1-970 | 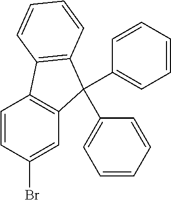 [SMILES: Brc1ccc2c(c1)C(c1ccccc1)(c1ccccc1)c1ccccc1-2] | | 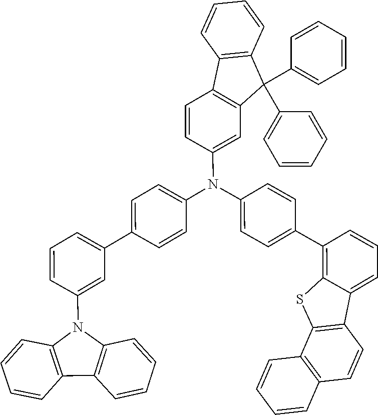 | 4.69 | 49 | 99.94 | 959.34 |
| Com- pound 1-971 | 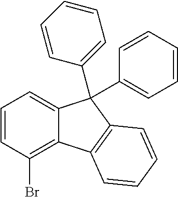 [SMILES: Brc1cccc2c1-c1ccccc1C2(c1ccccc1)c1ccccc1] | | 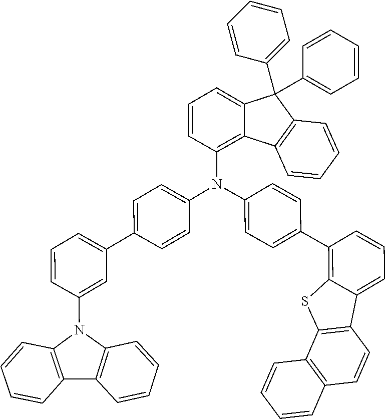 | 6.52 | 68 | 99.96 | 959.34 |
| Com- pound 1-972 | 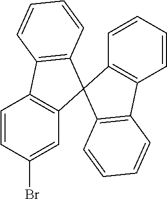 [SMILES: Brc1ccc2c(c1)C1(c3ccccc3-c3ccccc31)c1ccccc1-2] | | 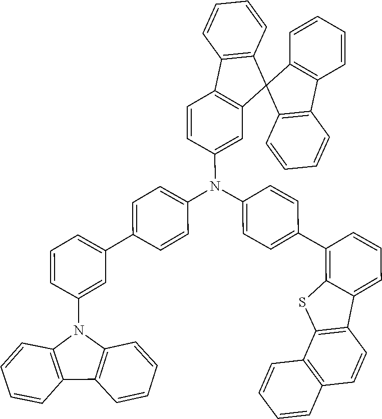 | 5.56 | 58 | 99.95 | 957.33 |
| Com- pound 1-973 | 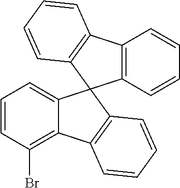 [SMILES: Brc1cccc2c1-c1ccccc1C21c2ccccc2-c2ccccc21] | | 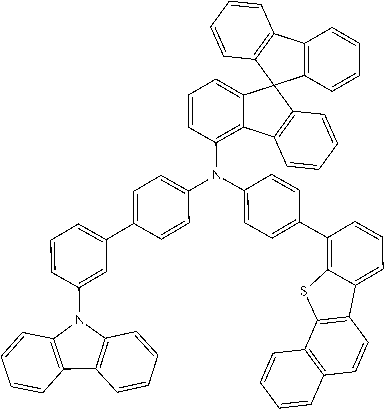 | 5.56 | 58 | 99.97 | 957.33 |
| Com- pound 1-987 | 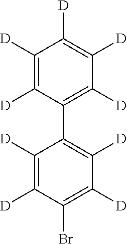 [SMILES: [2H]c1c([2H])c([2H])c(-c2c([2H])c([2H])c(Br)c([2H])c2[2H])c([2H])c1[2H]] | | 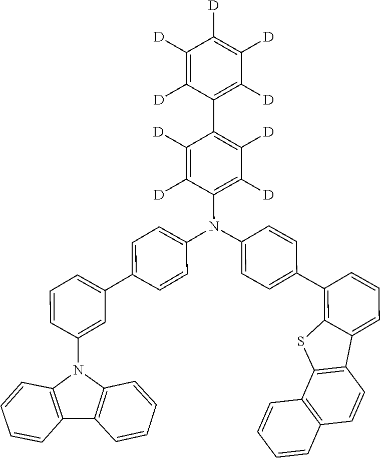 | 4.58 | 57 | 99.92 | 804.34 |
| Com- pound 1-988 | 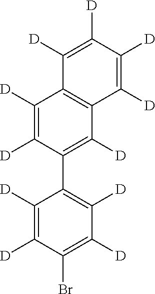 [SMILES: [2H]c1c([2H])c(-c2c([2H])c([2H])c3c([2H])c([2H])c([2H])c([2H])c3c2[2H])c([2H])c([2H])c1Br] | | 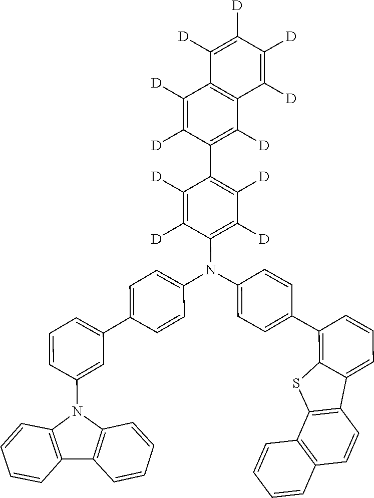 | 5.22 | 61 | 99.95 | 856.36 |
| Com- pound 1-147 | 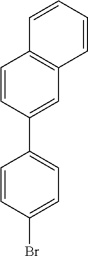 [SMILES: Brc1ccc(-c2ccc3ccccc3c2)cc1] | 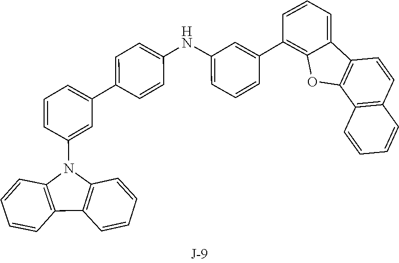 | 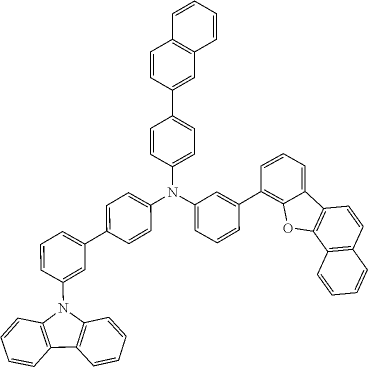 | 4.97 | 60 | 99.94 | 829.32 |
| Com- pound 1-732 | 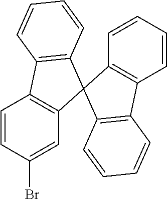 [SMILES: Brc1ccc2c(c1)C1(c3ccccc3-c3ccccc31)c1ccccc1-2] | | 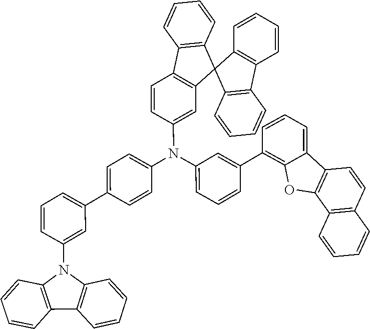 | 4.90 | 52 | 99.96 | 941.35 |
| Com- pound 1-733 | 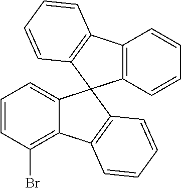 [SMILES: Brc1cccc2c1-c1ccccc1C21c2ccccc2-c2ccccc21] | | 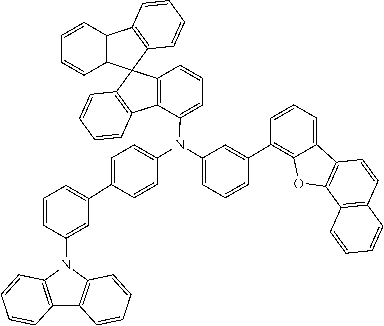 [SMILES: C1=CC2c3ccccc3C3(c4ccccc4-c4c(N(c5ccc(-c6cccc(-n7c8ccccc8c8ccccc87)c6)cc5)c5cccc(-c6cccc7c6oc6c8ccccc8ccc76)c5)cccc43)C2C=C1] | 5.56 | 59 | 99.93 | 941.36 |
| Com- pound 1-989 | 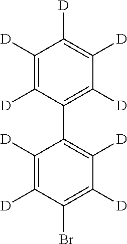 [SMILES: [2H]c1c([2H])c([2H])c(-c2c([2H])c([2H])c(Br)c([2H])c2[2H])c([2H])c1[2H]] | | 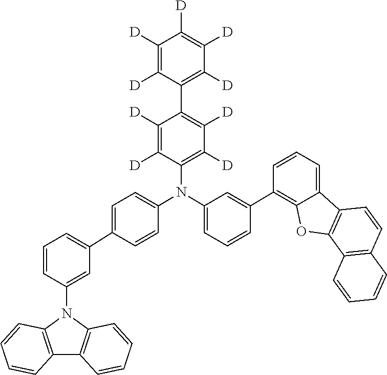 [SMILES: [2H]c1c([2H])c([2H])c(-c2c([2H])c([2H])c(N(c3ccc(-c4cccc(-n5c6ccccc6c6ccccc65)c4)cc3)c3cccc(-c4cccc5c4oc4c6ccccc6ccc54)c3)c([2H])c2[2H])c([2H])c1[2H]] | 4.49 | 57 | 99.95 | 788.37 |
| Com- pound 1-829 | 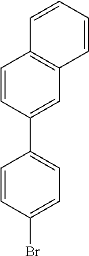 [SMILES: Brc1ccc(-c2ccc3ccccc3c2)cc1] | 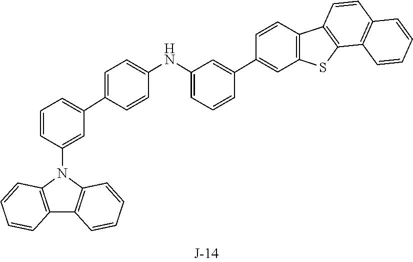 [SMILES: c1cc(Nc2ccc(-c3cccc(-n4c5ccccc5c5ccccc54)c3)cc2)cc(-c2ccc3c(c2)sc2c4ccccc4ccc32)c1] | 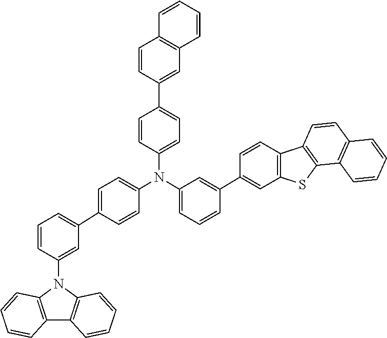 [SMILES: c1cc(-c2ccc3c(c2)sc2c4ccccc4ccc32)cc(N(c2ccc(-c3cccc(-n4c5ccccc5c5ccccc54)c3)cc2)c2ccc(-c3ccc4ccccc4c3)cc2)c1] | 5.07 | 60 | 99.93 | 845.29 |
| Com- pound 2-1 | 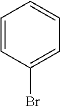 | 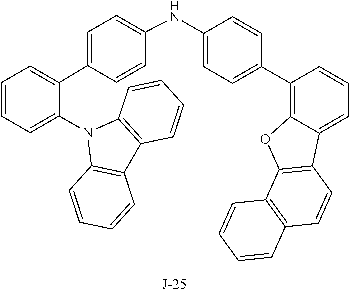 [SMILES: c1ccc(-n2c3ccccc3c3ccccc32)c(-c2ccc(Nc3ccc(-c4cccc5c4oc4c6ccccc6ccc54)cc3)cc2)c1] | 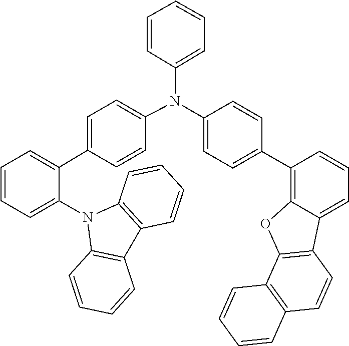 [SMILES: c1ccc(N(c2ccc(-c3ccccc3-n3c4ccccc4c4ccccc43)cc2)c2ccc(-c3cccc4c3oc3c5ccccc5ccc43)cc2)cc1] | 3.72 | 53 | 99.96 | 703.27 |
| Com- pound 2-25 | 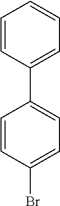 [SMILES: Brc1ccc(-c2ccccc2)cc1] | | 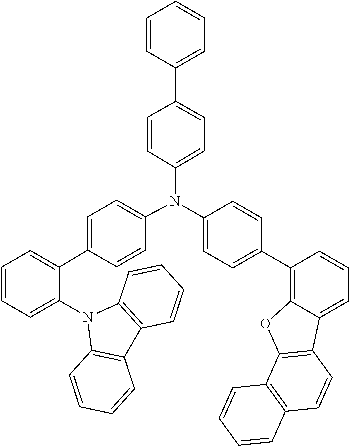 [SMILES: c1ccc(-c2ccc(N(c3ccc(-c4ccccc4-n4c5ccccc5c5ccccc54)cc3)c3ccc(-c4cccc5c4oc4c6ccccc6ccc54)cc3)cc2)cc1] | 4.44 | 57 | 99.95 | 779.30 |
| Com- pound 2-26 | 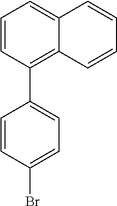 [SMILES: Brc1ccc(-c2cccc3ccccc23)cc1] | | 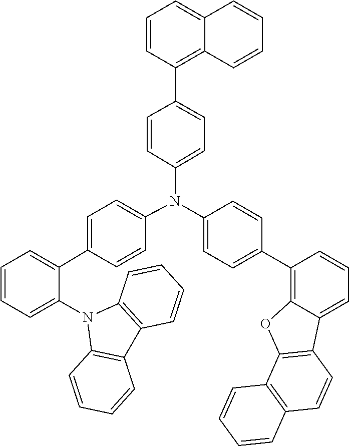 [SMILES: c1ccc(-n2c3ccccc3c3ccccc32)c(-c2ccc(N(c3ccc(-c4cccc5ccccc45)cc3)c3ccc(-c4cccc5c4oc4c6ccccc6ccc54)cc3)cc2)c1] | 4.47 | 54 | 99.94 | 829.32 |
| Com- pound 2-27 | 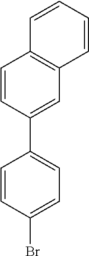 [SMILES: Brc1ccc(-c2ccc3ccccc3c2)cc1] | | 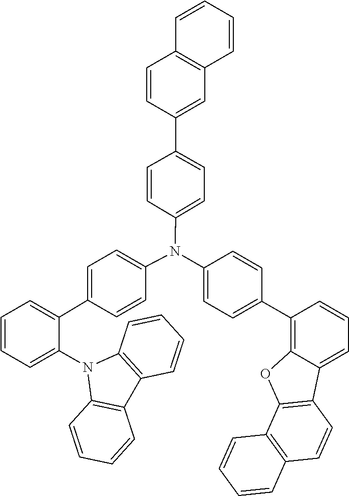 [SMILES: c1ccc(-n2c3ccccc3c3ccccc32)c(-c2ccc(N(c3ccc(-c4ccc5ccccc5c4)cc3)c3ccc(-c4cccc5c4oc4c6ccccc6ccc54)cc3)cc2)c1] | 4.14 | 50 | 99.90 | 829.31 |
| Com- pound 2-28 | 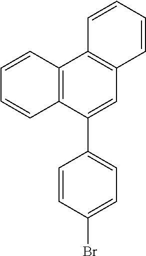 [SMILES: Brc1ccc(-c2cc3ccccc3c3ccccc23)cc1] | | 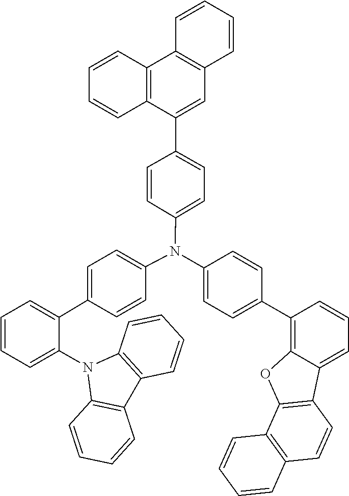 [SMILES: c1ccc(-n2c3ccccc3c3ccccc32)c(-c2ccc(N(c3ccc(-c4cc5ccccc5c5ccccc45)cc3)c3ccc(-c4cccc5c4oc4c6ccccc6ccc54)cc3)cc2)c1] | 5.09 | 58 | 99.96 | 879.33 |
| Com- pound 2-145 | 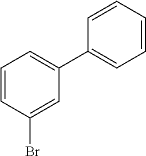 [SMILES: Brc1cccc(-c2ccccc2)c1] | | 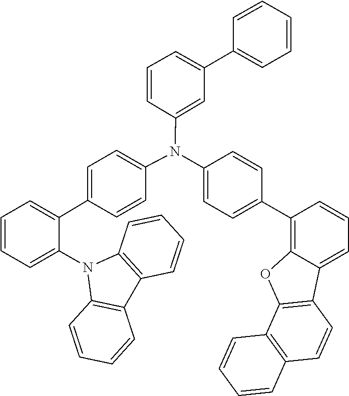 [SMILES: c1ccc(-c2cccc(N(c3ccc(-c4ccccc4-n4c5ccccc5c5ccccc54)cc3)c3ccc(-c4cccc5c4oc4c6ccccc6ccc54)cc3)c2)cc1] | 4.05 | 52 | 99.96 | 779.30 |
| Com- pound 2-146 | 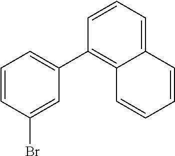 [SMILES: Brc1cccc(-c2cccc3ccccc23)c1] | | 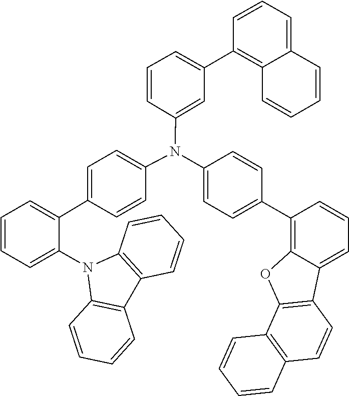 [SMILES: c1cc(-c2cccc3ccccc23)cc(N(c2ccc(-c3ccccc3-n3c4ccccc4c4ccccc43)cc2)c2ccc(-c3cccc4c3oc3c5ccccc5ccc43)cc2)c1] | 4.80 | 58 | 99.94 | 829.32 |
| Com- pound 2-147 | 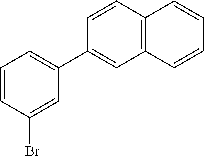 [SMILES: Brc1cccc(-c2ccc3ccccc3c2)c1] | | 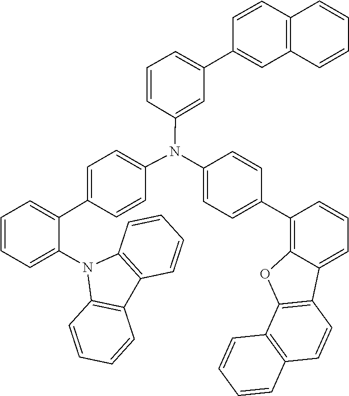 [SMILES: c1cc(-c2ccc3ccccc3c2)cc(N(c2ccc(-c3ccccc3-n3c4ccccc4c4ccccc43)cc2)c2ccc(-c3cccc4c3oc3c5ccccc5ccc43)cc2)c1] | 5.38 | 65 | 99.96 | 829.33 |
| Com- pound 2-49 | 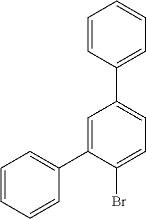 [SMILES: Brc1ccc(-c2ccccc2)cc1-c1ccccc1] | | 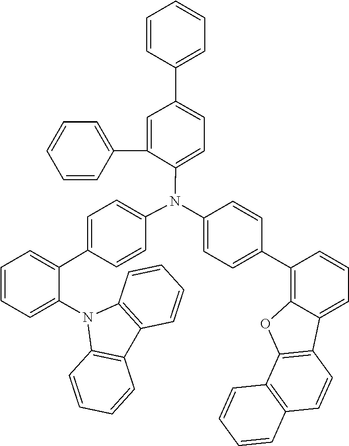 [SMILES: c1ccc(-c2ccc(N(c3ccc(-c4ccccc4-n4c5ccccc5c5ccccc54)cc3)c3ccc(-c4cccc5c4oc4c6ccccc6ccc54)cc3)c(-c3ccccc3)c2)cc1] | 5.47 | 64 | 99.97 | 855.33 |
| Com- pound 2-97 | 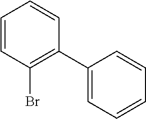 [SMILES: Brc1ccccc1-c1ccccc1] | | 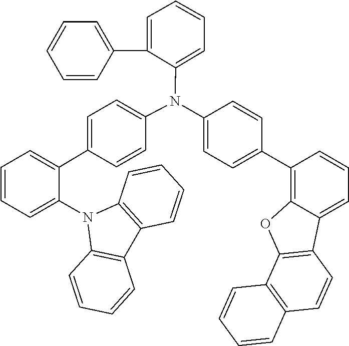 [SMILES: c1ccc(-c2ccccc2N(c2ccc(-c3ccccc3-n3c4ccccc4c4ccccc43)cc2)c2ccc(-c3cccc4c3oc3c5ccccc5ccc43)cc2)cc1] | 4.67 | 60 | 99.92 | 779.31 |
| Com- pound 2-51 | 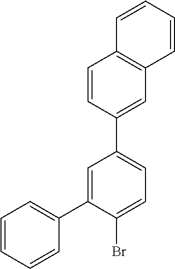 [SMILES: Brc1ccc(-c2ccc3ccccc3c2)cc1-c1ccccc1] | | 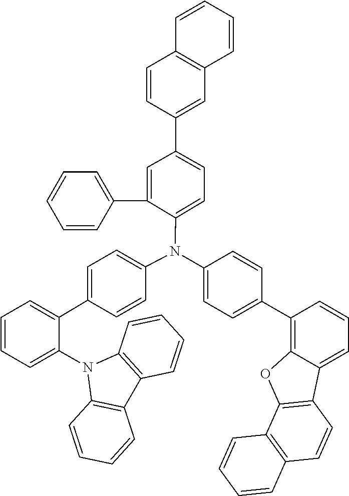 [SMILES: c1ccc(-c2cc(-c3ccc4ccccc4c3)ccc2N(c2ccc(-c3ccccc3-n3c4ccccc4c4ccccc43)cc2)c2ccc(-c3cccc4c3oc3c5ccccc5ccc43)cc2)cc1] | 4.70 | 52 | 99.93 | 905.35 |
| Com- pound 2-121 | 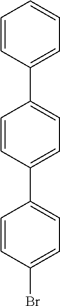 [SMILES: Brc1ccc(-c2ccc(-c3ccccc3)cc2)cc1] | | 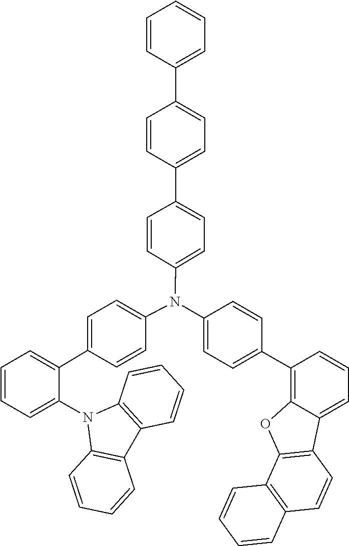 [SMILES: c1ccc(-c2ccc(-c3ccc(N(c4ccc(-c5ccccc5-n5c6ccccc6c6ccccc65)cc4)c4ccc(-c5cccc6c5oc5c7ccccc7ccc65)cc4)cc3)cc2)cc1] | 4.96 | 58 | 99.90 | 855.33 |
| Com- pound 2-169 | 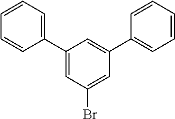 [SMILES: Brc1cc(-c2ccccc2)cc(-c2ccccc2)c1] | | 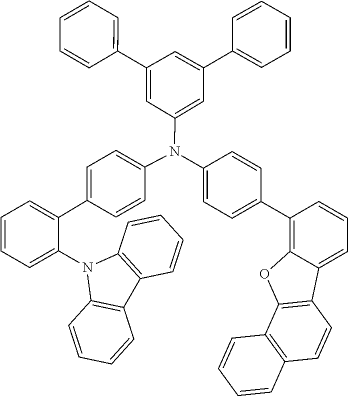 [SMILES: c1ccc(-c2cc(-c3ccccc3)cc(N(c3ccc(-c4ccccc4-n4c5ccccc5c5ccccc54)cc3)c3ccc(-c4cccc5c4oc4c6ccccc6ccc54)cc3)c2)cc1] | 4.53 | 53 | 99.98 | 855.33 |
| Com- pound 2-170 | 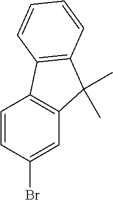 [SMILES: CC1(C)c2ccccc2-c2ccc(Br)cc21] | | 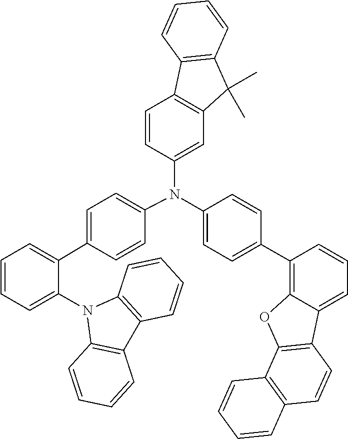 [SMILES: CC1(C)c2ccccc2-c2ccc(N(c3ccc(-c4ccccc4-n4c5ccccc5c5ccccc54)cc3)c3ccc(-c4cccc5c4oc4c6ccccc6ccc54)cc3)cc21] | 4.91 | 60 | 99.93 | 819.33 |
| Com- pound 2-171 | 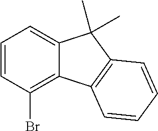 [SMILES: CC1(C)c2ccccc2-c2c(Br)cccc21] | | 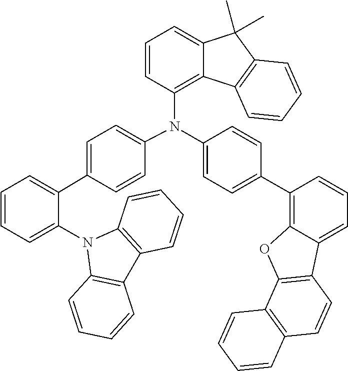 [SMILES: CC1(C)c2ccccc2-c2c(N(c3ccc(-c4ccccc4-n4c5ccccc5c5ccccc54)cc3)c3ccc(-c4cccc5c4oc4c6ccccc6ccc54)cc3)cccc21] | 5.24 | 64 | 99.94 | 819.33 |
| Com- pound 2-172 | 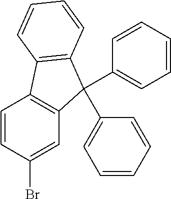 [SMILES: Brc1ccc2c(c1)C(c1ccccc1)(c1ccccc1)c1ccccc1-2] | | 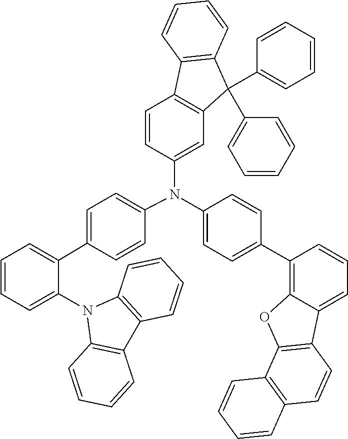 [SMILES: c1ccc(C2(c3ccccc3)c3ccccc3-c3ccc(N(c4ccc(-c5ccccc5-n5c6ccccc6c6ccccc65)cc4)c4ccc(-c5cccc6c5oc5c7ccccc7ccc65)cc4)cc32)cc1] | 5.84 | 62 | 99.90 | 943.35 |
| Com- pound 2-173 | 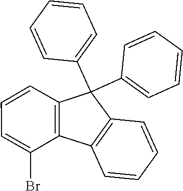 [SMILES: Brc1cccc2c1-c1ccccc1C2(c1ccccc1)c1ccccc1] | | 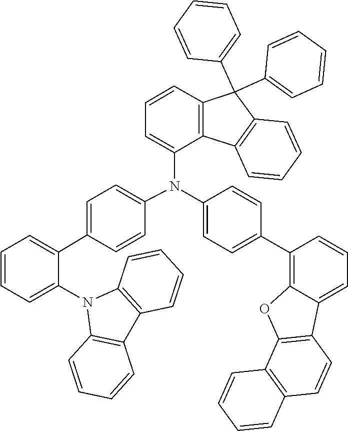 [SMILES: c1ccc(C2(c3ccccc3)c3ccccc3-c3c(N(c4ccc(-c5ccccc5-n5c6ccccc6c6ccccc65)cc4)c4ccc(-c5cccc6c5oc5c7ccccc7ccc65)cc4)cccc32)cc1] | 6.41 | 68 | 99.92 | 943.36 |
| Com- pound 2-174 | 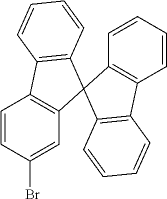 [SMILES: Brc1ccc2c(c1)C1(c3ccccc3-c3ccccc31)c1ccccc1-2] | | 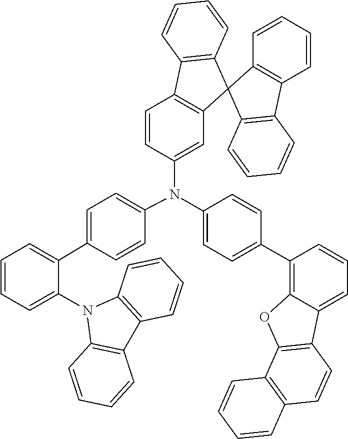 [SMILES: c1ccc(-n2c3ccccc3c3ccccc32)c(-c2ccc(N(c3ccc(-c4cccc5c4oc4c6ccccc6ccc54)cc3)c3ccc4c(c3)C3(c5ccccc5-c5ccccc53)c3ccccc3-4)cc2)c1] | 5.47 | 58 | 99.95 | 941.35 |
| Com- pound 2-181 | 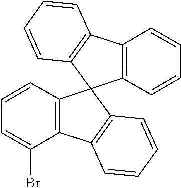 [SMILES: Brc1cccc2c1-c1ccccc1C21c2ccccc2-c2ccccc21] | | 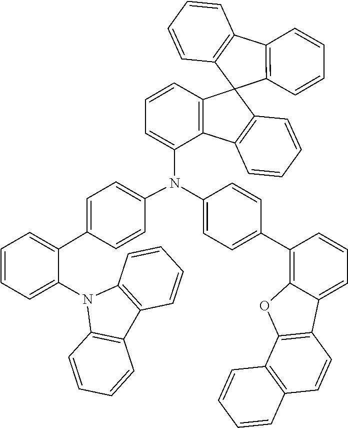 [SMILES: c1ccc(-n2c3ccccc3c3ccccc32)c(-c2ccc(N(c3ccc(-c4cccc5c4oc4c6ccccc6ccc54)cc3)c3cccc4c3-c3ccccc3C43c4ccccc4-c4ccccc43)cc2)c1] | 5.09 | 54 | 99.97 | 941.35 |
| Com- pound 2-37 | 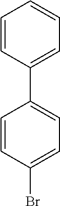 [SMILES: Brc1ccc(-c2ccccc2)cc1] | 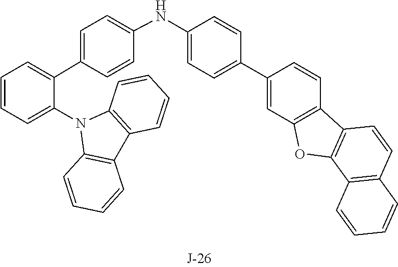 [SMILES: c1ccc(-n2c3ccccc3c3ccccc32)c(-c2ccc(Nc3ccc(-c4ccc5c(c4)oc4c6ccccc6ccc54)cc3)cc2)c1] | 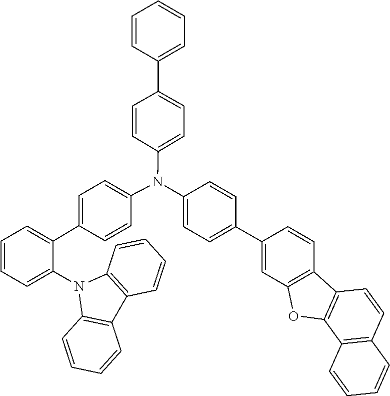 [SMILES: c1ccc(-c2ccc(N(c3ccc(-c4ccc5c(c4)oc4c6ccccc6ccc54)cc3)c3ccc(-c4ccccc4-n4c5ccccc5c5ccccc54)cc3)cc2)cc1] | 5.06 | 65 | 99.94 | 779.31 |
| Com- pound 2-38 | 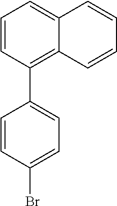 [SMILES: Brc1ccc(-c2cccc3ccccc23)cc1] | | 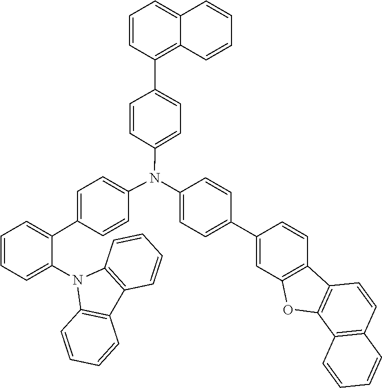 [SMILES: c1ccc(-n2c3ccccc3c3ccccc32)c(-c2ccc(N(c3ccc(-c4ccc5c(c4)oc4c6ccccc6ccc54)cc3)c3ccc(-c4cccc5ccccc45)cc3)cc2)c1] | 5.30 | 64 | 99.96 | 829.33 |
| Com- pound 2-45 | 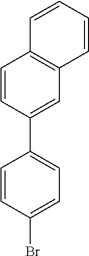 [SMILES: Brc1ccc(-c2ccc3ccccc3c2)cc1] | | 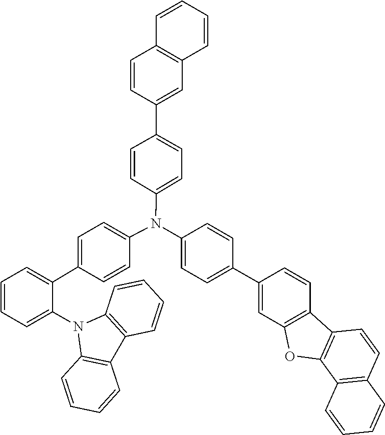 [SMILES: c1ccc(-n2c3ccccc3c3ccccc32)c(-c2ccc(N(c3ccc(-c4ccc5ccccc5c4)cc3)c3ccc(-c4ccc5c(c4)oc4c6ccccc6ccc54)cc3)cc2)c1] | 4.31 | 52 | 99.92 | 829.32 |
| Com- pound 2-85 | 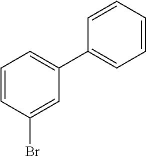 [SMILES: Brc1cccc(-c2ccccc2)c1] | | 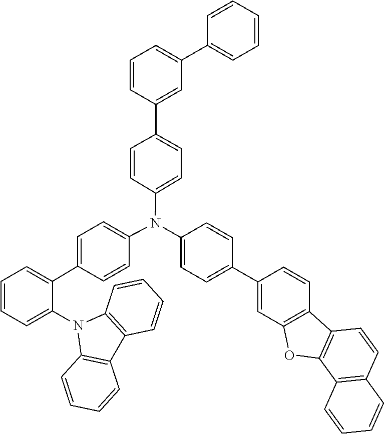 [SMILES: c1ccc(-c2cccc(-c3ccc(N(c4ccc(-c5ccc6c(c5)oc5c7ccccc7ccc65)cc4)c4ccc(-c5ccccc5-n5c6ccccc6c6ccccc65)cc4)cc3)c2)cc1] | 4.36 | 56 | 99.91 | 779.30 |
| Com- pound 2-86 | 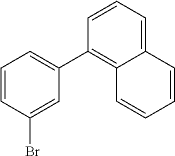 [SMILES: Brc1cccc(-c2cccc3ccccc23)c1] | | 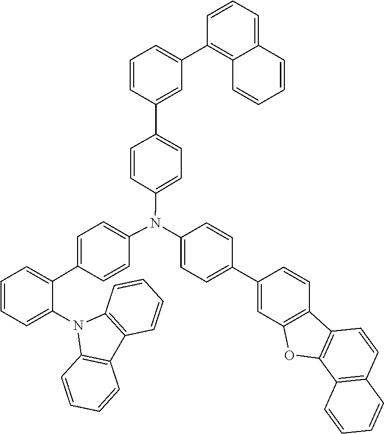 [SMILES: c1cc(-c2ccc(N(c3ccc(-c4ccc5c(c4)oc4c6ccccc6ccc54)cc3)c3ccc(-c4ccccc4-n4c5ccccc5c5ccccc54)cc3)cc2)cc(-c2cccc3ccccc23)c1] | 4.47 | 54 | 99.97 | 829.32 |
| Com- pound 2-87 | 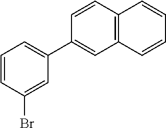 [SMILES: Brc1cccc(-c2ccc3ccccc3c2)c1] | | 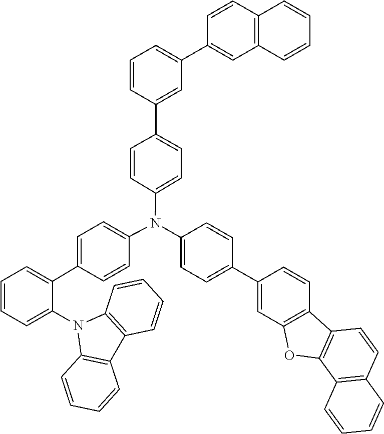 [SMILES: c1cc(-c2ccc(N(c3ccc(-c4ccc5c(c4)oc4c6ccccc6ccc54)cc3)c3ccc(-c4ccccc4-n4c5ccccc5c5ccccc54)cc3)cc2)cc(-c2ccc3ccccc3c2)c1] | 4.80 | 58 | 99.93 | 829.32 |
| 2-61 | 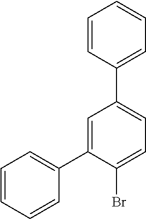 [SMILES: Brc1ccc(-c2ccccc2)cc1-c1ccccc1] | | 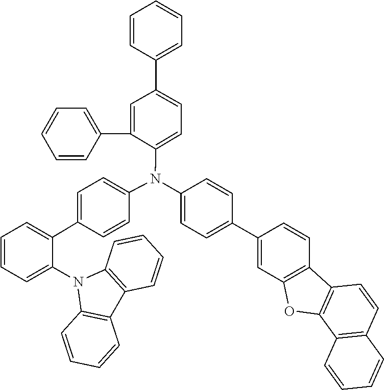 [SMILES: c1ccc(-c2ccc(N(c3ccc(-c4ccc5c(c4)oc4c6ccccc6ccc54)cc3)c3ccc(-c4ccccc4-n4c5ccccc5c5ccccc54)cc3)c(-c3ccccc3)c2)cc1] | 5.04 | 59 | 99.90 | 855.33 |
| Com- pound 2-109 | 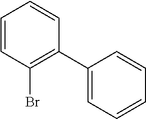 [SMILES: Brc1ccccc1-c1ccccc1] | | 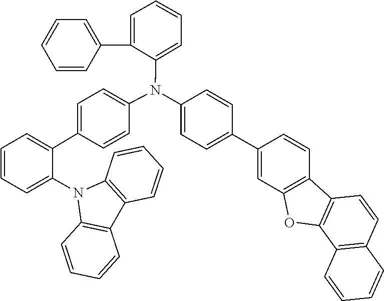 [SMILES: c1ccc(-c2ccccc2N(c2ccc(-c3ccc4c(c3)oc3c5ccccc5ccc43)cc2)c2ccc(-c3ccccc3-n3c4ccccc4c4ccccc43)cc2)cc1] | 4.36 | 56 | 99.93 | 779.30 |
| Com- pound 2-133 | 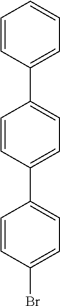 [SMILES: Brc1ccc(-c2ccc(-c3ccccc3)cc2)cc1] | | 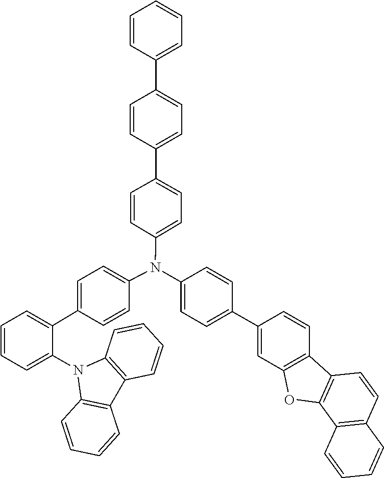 [SMILES: c1ccc(-c2ccc(-c3ccc(N(c4ccc(-c5ccc6c(c5)oc5c7ccccc7ccc65)cc4)c4ccc(-c5ccccc5-n5c6ccccc6c6ccccc65)cc4)cc3)cc2)cc1] | 4.95 | 58 | 99.90 | 855.33 |
| Com- pound 2-182 | 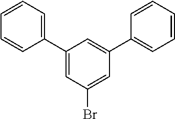 [SMILES: Brc1cc(-c2ccccc2)cc(-c2ccccc2)c1] | | 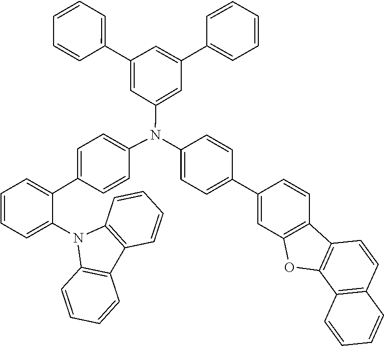 [SMILES: c1ccc(-c2cc(-c3ccccc3)cc(N(c3ccc(-c4ccc5c(c4)oc4c6ccccc6ccc54)cc3)c3ccc(-c4ccccc4-n4c5ccccc5c5ccccc54)cc3)c2)cc1] | 4.44 | 52 | 99.91 | 855.34 |
| Com- pound 2-193 | 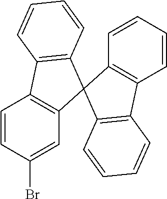 [SMILES: Brc1ccc2c(c1)C1(c3ccccc3-c3ccccc31)c1ccccc1-2] | | 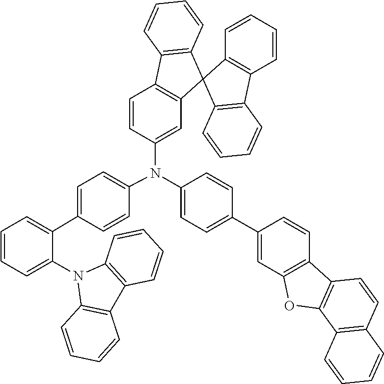 [SMILES: c1ccc(-n2c3ccccc3c3ccccc32)c(-c2ccc(N(c3ccc(-c4ccc5c(c4)oc4c6ccccc6ccc54)cc3)c3ccc4c(c3)C3(c5ccccc5-c5ccccc53)c3ccccc3-4)cc2)c1] | 5.37 | 57 | 99.95 | 941.36 |
| Com- pound 2-194 | 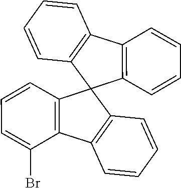 [SMILES: Brc1cccc2c1-c1ccccc1C21c2ccccc2-c2ccccc21] | | 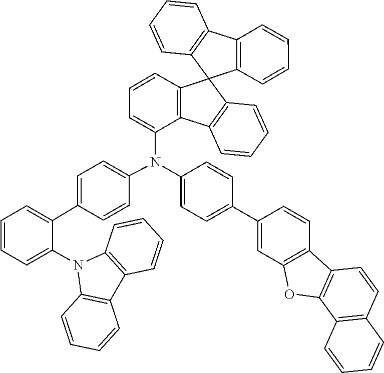 [SMILES: c1ccc(-n2c3ccccc3c3ccccc32)c(-c2ccc(N(c3ccc(-c4ccc5c(c4)oc4c6ccccc6ccc54)cc3)c3cccc4c3-c3ccccc3C43c4ccccc4-c4ccccc43)cc2)c1] | 5.75 | 61 | 99.91 | 941.35 |
| Com- pound 2-197 | 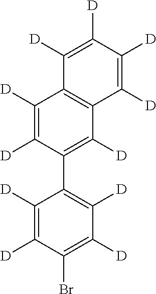 [SMILES: [2H]c1c([2H])c(-c2c([2H])c([2H])c3c([2H])c([2H])c([2H])c([2H])c3c2[2H])c([2H])c([2H])c1Br] | | 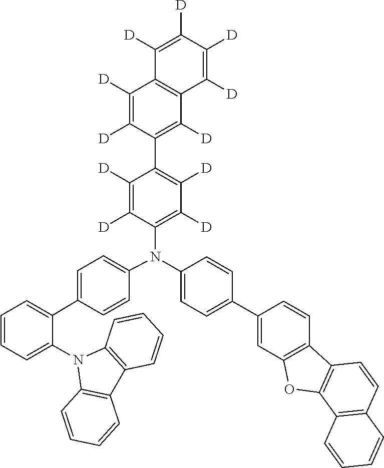 [SMILES: [2H]c1c([2H])c(N(c2ccc(-c3ccc4c(c3)oc3c5ccccc5ccc43)cc2)c2ccc(-c3ccccc3-n3c4ccccc4c4ccccc43)cc2)c([2H])c([2H])c1-c1c([2H])c([2H])c2c([2H])c([2H])c([2H])c([2H])c2c1[2H]] | 4.87 | 58 | 99.95 | 840.39 |
| Com- pound 2-31 | 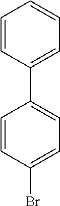 [SMILES: Brc1ccc(-c2ccccc2)cc1] | 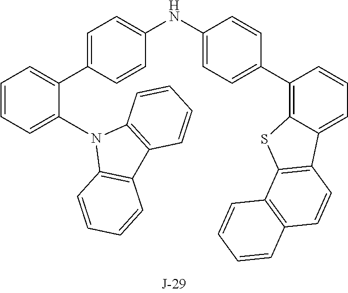 [SMILES: c1ccc(-n2c3ccccc3c3ccccc32)c(-c2ccc(Nc3ccc(-c4cccc5c4sc4c6ccccc6ccc54)cc3)cc2)c1] | 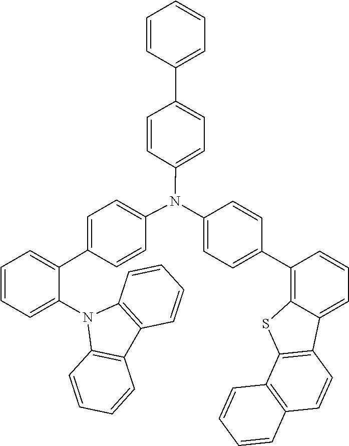 [SMILES: c1ccc(-c2ccc(N(c3ccc(-c4ccccc4-n4c5ccccc5c5ccccc54)cc3)c3ccc(-c4cccc5c4sc4c6ccccc6ccc54)cc3)cc2)cc1] | 4.77 | 60 | 99.94 | 795.28 |
| Com- pound 2-32 | 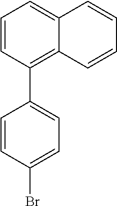 [SMILES: Brc1ccc(-c2cccc3ccccc23)cc1] | | 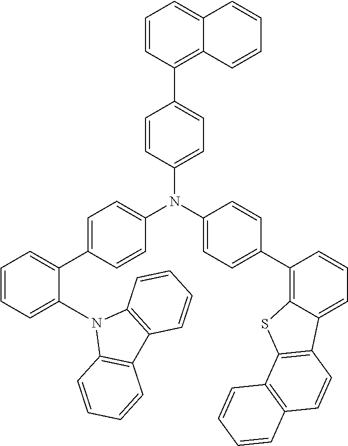 [SMILES: c1ccc(-n2c3ccccc3c3ccccc32)c(-c2ccc(N(c3ccc(-c4cccc5ccccc45)cc3)c3ccc(-c4cccc5c4sc4c6ccccc6ccc54)cc3)cc2)c1] | 5.24 | 62 | 99.95 | 845.30 |
| Com- pound 2-22 | 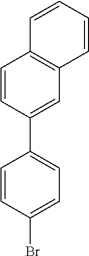 [SMILES: Brc1ccc(-c2ccc3ccccc3c2)cc1] | | 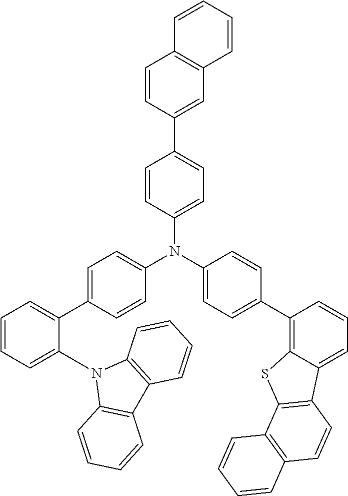 [SMILES: c1ccc(-n2c3ccccc3c3ccccc32)c(-c2ccc(N(c3ccc(-c4ccc5ccccc5c4)cc3)c3ccc(-c4cccc5c4sc4c6ccccc6ccc54)cc3)cc2)c1] | 5.40 | 64 | 99.96 | 845.29 |
| Com- pound 2-79 | 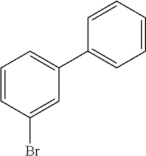 [SMILES: Brc1cccc(-c2ccccc2)c1] | | 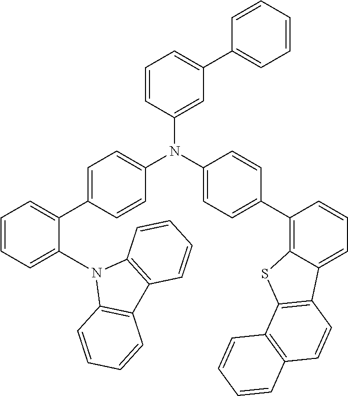 [SMILES: c1ccc(-c2cccc(N(c3ccc(-c4ccccc4-n4c5ccccc5c5ccccc54)cc3)c3ccc(-c4cccc5c4sc4c6ccccc6ccc54)cc3)c2)cc1] | 4.61 | 58 | 99.96 | 795.28 |
| Com- pound 2-55 | 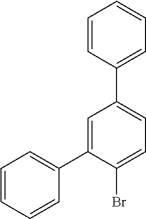 [SMILES: Brc1ccc(-c2ccccc2)cc1-c1ccccc1] | | 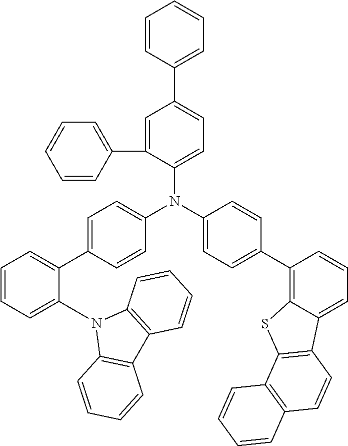 [SMILES: c1ccc(-c2ccc(N(c3ccc(-c4ccccc4-n4c5ccccc5c5ccccc54)cc3)c3ccc(-c4cccc5c4sc4c6ccccc6ccc54)cc3)c(-c3ccccc3)c2)cc1] | 4.53 | 52 | 99.95 | 871.31 |
| Com- pound 2-198 | 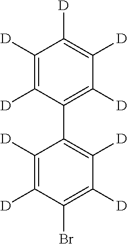 [SMILES: [2H]c1c([2H])c([2H])c(-c2c([2H])c([2H])c(Br)c([2H])c2[2H])c([2H])c1[2H]] | | 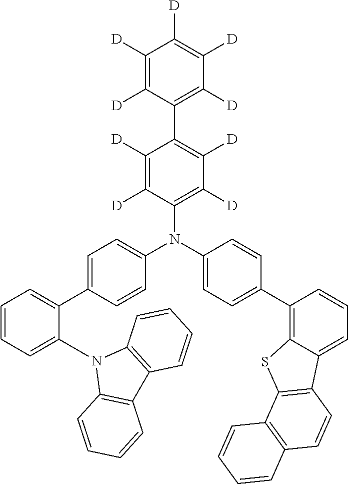 [SMILES: [2H]c1c([2H])c([2H])c(-c2c([2H])c([2H])c(N(c3ccc(-c4ccccc4-n4c5ccccc5c5ccccc54)cc3)c3ccc(-c4cccc5c4sc4c6ccccc6ccc54)cc3)c([2H])c2[2H])c([2H])c1[2H]] | 4.10 | 51 | 99.92 | 804.34 |
| Com- pound 2-199 | 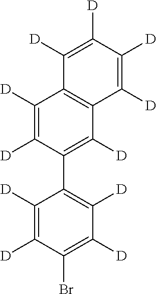 [SMILES: [2H]c1c([2H])c(-c2c([2H])c([2H])c3c([2H])c([2H])c([2H])c([2H])c3c2[2H])c([2H])c([2H])c1Br] | | 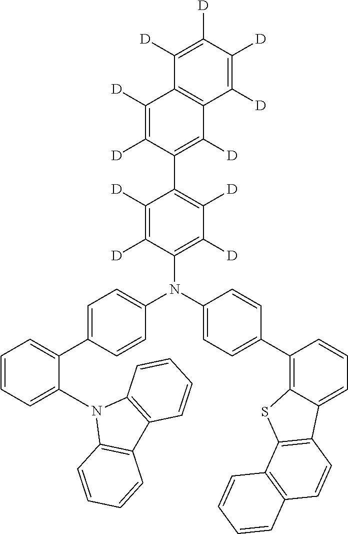 [SMILES: [2H]c1c([2H])c(N(c2ccc(-c3ccccc3-n3c4ccccc4c4ccccc43)cc2)c2ccc(-c3cccc4c3sc3c5ccccc5ccc43)cc2)c([2H])c([2H])c1-c1c([2H])c([2H])c2c([2H])c([2H])c([2H])c([2H])c2c1[2H]] | 4.28 | 50 | 99.98 | 856.37 |
| Com- pound 1-1005 | 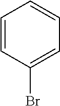 | 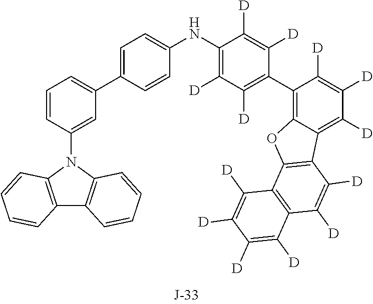 | 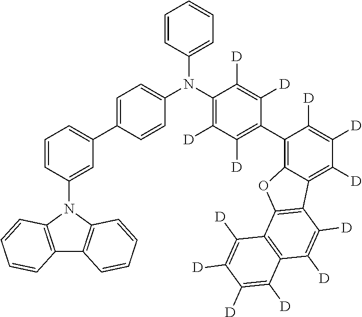 | 4.00 | 56 | 99.95 | 716.35 |
| Com- pound 1-1006 | 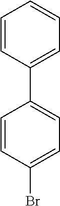 [SMILES: Brc1ccc(-c2ccccc2)cc1] | | 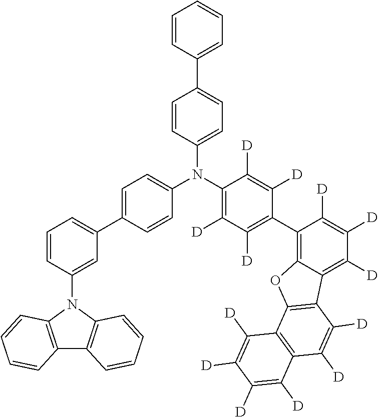 | 4.67 | 59 | 99.94 | 792.38 |
| Com- pound 1-1007 | 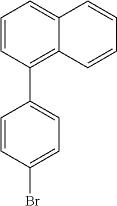 [SMILES: Brc1ccc(-c2cccc3ccccc23)cc1] | | 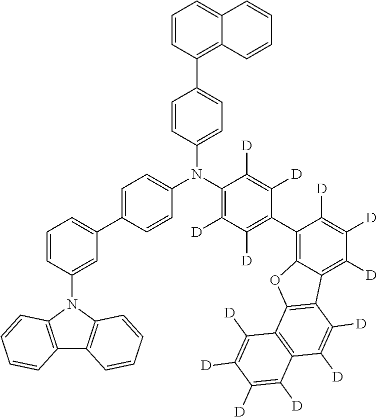 | 4.04 | 48 | 99.90 | 842.40 |
| Com- pound 1-993 | 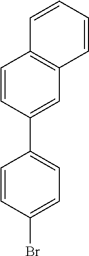 [SMILES: Brc1ccc(-c2ccc3ccccc3c2)cc1] | | 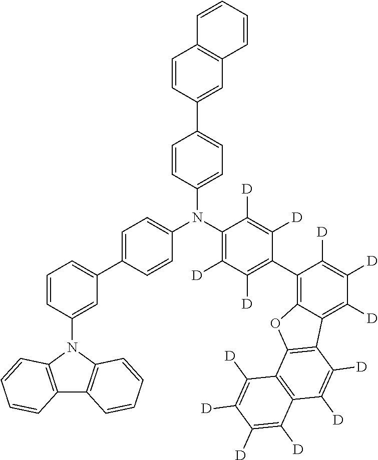 | 4.37 | 52 | 99.94 | 842.41 |
| Com- pound 1-994 | 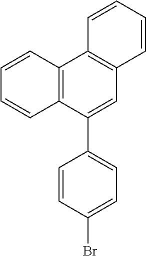 [SMILES: Brc1ccc(-c2cc3ccccc3c3ccccc23)cc1] | | 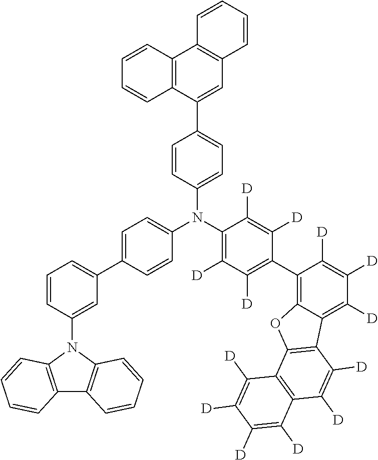 | 4.19 | 47 | 99.96 | 892.41 |
| Com- pound 1-995 | 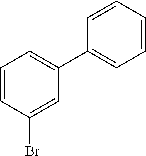 [SMILES: Brc1cccc(-c2ccccc2)c1] | | 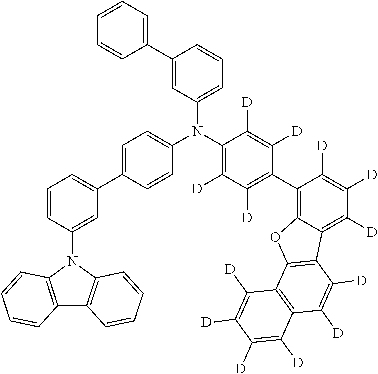 | 3.96 | 50 | 99.98 | 792.38 |
| Com- pound 1-996 | 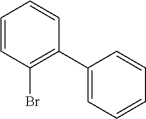 [SMILES: Brc1ccccc1-c1ccccc1] | | 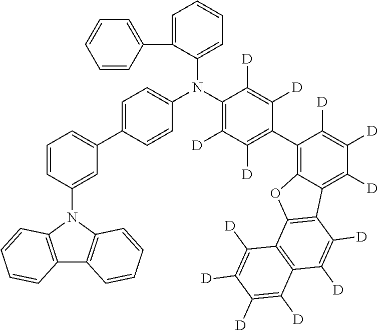 | 4.43 | 56 | 99.97 | 792.37 |
| Com- pound 1-997 | 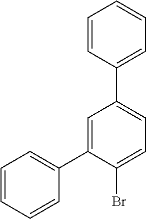 [SMILES: Brc1ccc(-c2ccccc2)cc1-c1ccccc1] | | 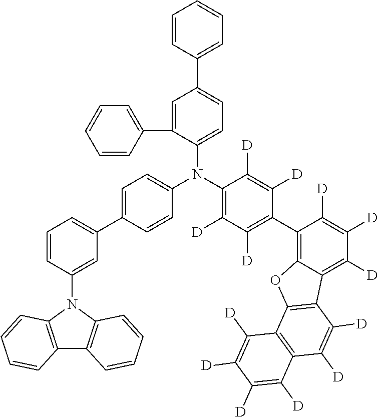 | 5.03 | 58 | 99.99 | 868.41 |
| Com- pound 1-998 | 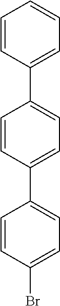 [SMILES: Brc1ccc(-c2ccc(-c3ccccc3)cc2)cc1] | | 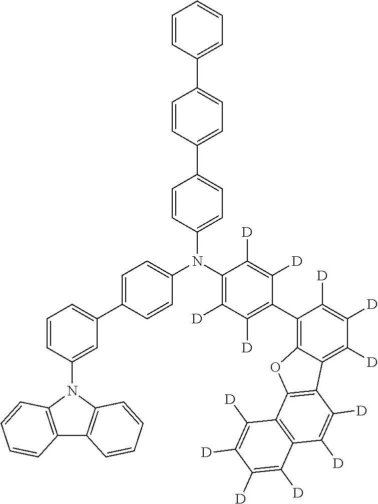 | 4.51 | 52 | 99.99 | 868.41 |
| Com- pound 1-999 | 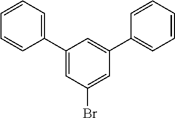 [SMILES: Brc1cc(-c2ccccc2)cc(-c2ccccc2)c1] | | 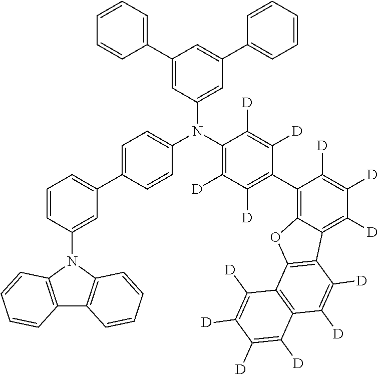 | 4.25 | 49 | 99.96 | 868.41 |
| Com- pound 1-1000 | 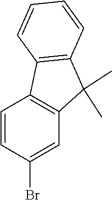 [SMILES: CC1(C)c2ccccc2-c2ccc(Br)cc21] | | 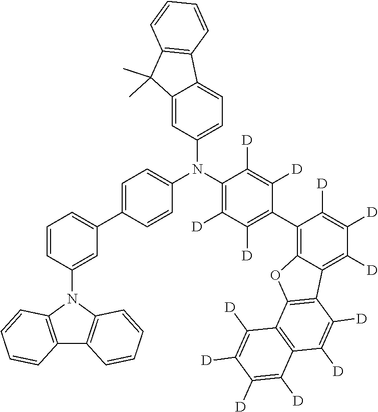 | 4.40 | 53 | 99.97 | 832.41 |
| Com- pound 1-1001 | 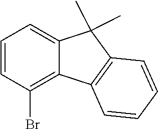 [SMILES: CC1(C)c2ccccc2-c2c(Br)cccc21] | | 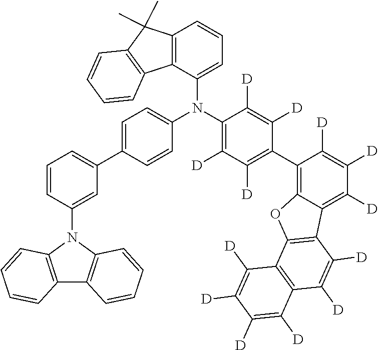 | 4.99 | 60 | 99.97 | 832.41 |
| Com- pound 1-1002 | 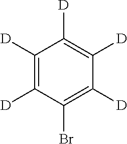 [SMILES: [2H]c1c([2H])c([2H])c(Br)c([2H])c1[2H]] | | 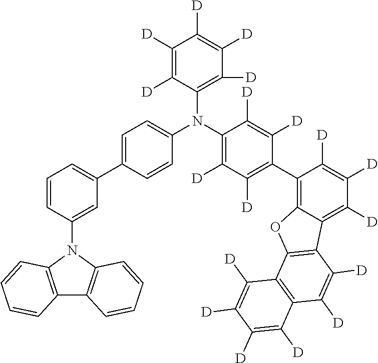 | 4.39 | 61 | 99.95 | 721.38 |
| Com- pound 1-1003 | 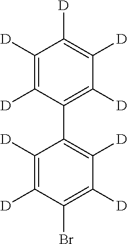 [SMILES: [2H]c1c([2H])c([2H])c(-c2c([2H])c([2H])c(Br)c([2H])c2[2H])c([2H])c1[2H]] | | 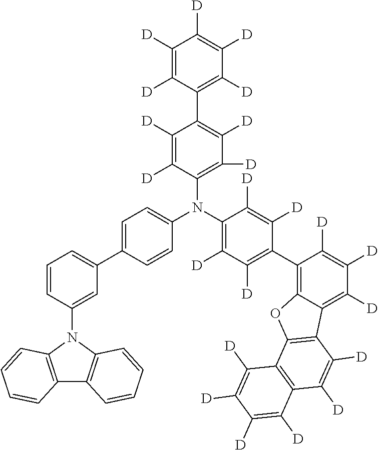 | 4.64 | 58 | 99.97 | 801.44 |
| Com- pound 1-1004 | 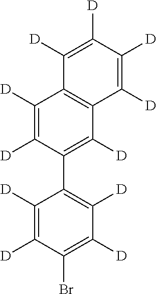 [SMILES: [2H]c1c([2H])c(-c2c([2H])c([2H])c3c([2H])c([2H])c([2H])c([2H])c3c2[2H])c([2H])c([2H])c1Br] | | 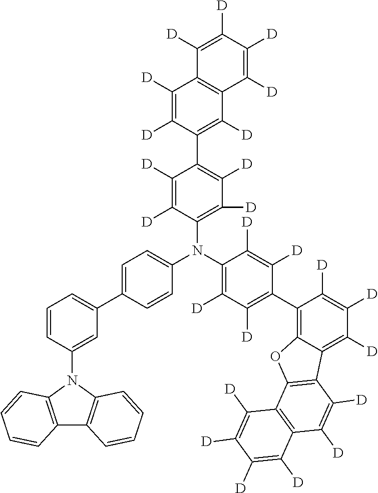 | 4.69 | 55 | 99.91 | 853.47 |
| Com- pound 2-200 | 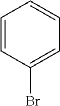 | 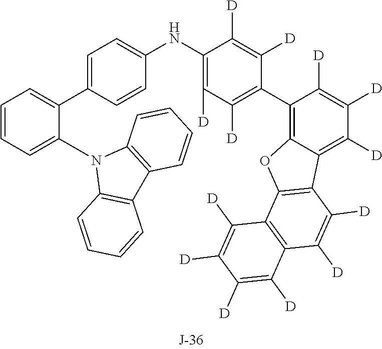 | 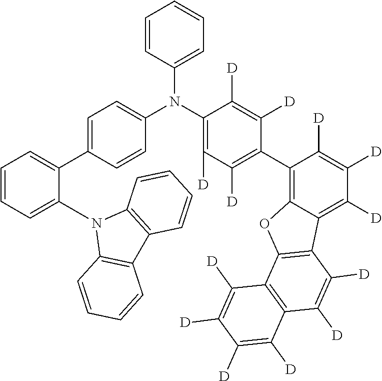 | 3.57 | 50 | 99.89 | 716.35 |
| Com- pound 2-201 | 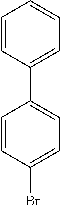 [SMILES: Brc1ccc(-c2ccccc2)cc1] | | 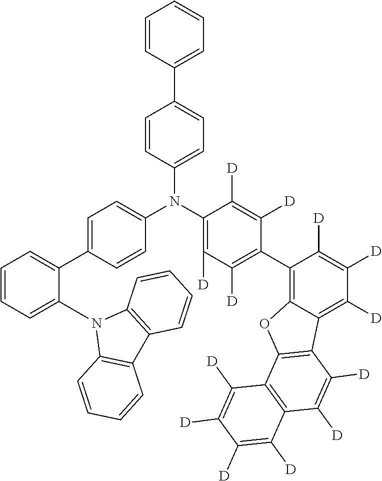 | 4.67 | 59 | 99.92 | 792.37 |
| Com- pound 2-202 | 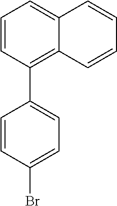 [SMILES: Brc1ccc(-c2cccc3ccccc23)cc1] | | 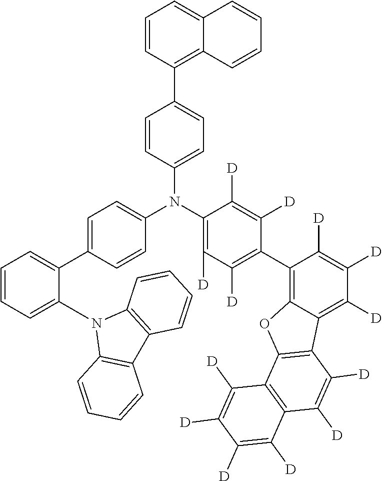 | 4.54 | 54 | 99.91 | 842.41 |
| Com- pound 2-203 | 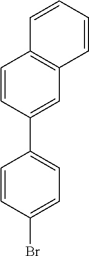 [SMILES: Brc1ccc(-c2ccc3ccccc3c2)cc1] | | 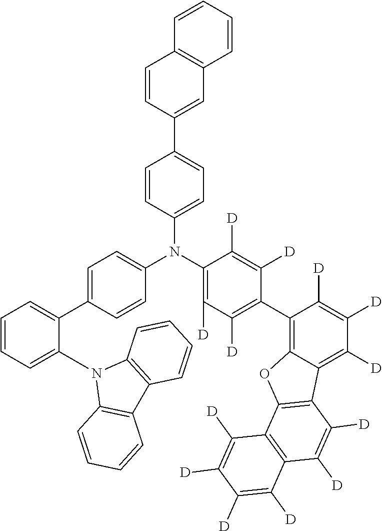 | 4.71 | 56 | 99.90 | 842.40 |
| Com- pound 2-204 | 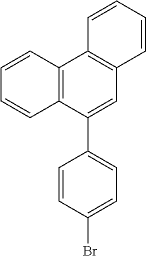 [SMILES: Brc1ccc(-c2cc3ccccc3c3ccccc23)cc1] | | 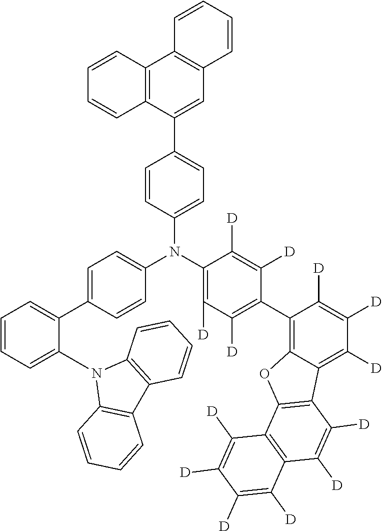 | 5.08 | 57 | 99.91 | 892.41 |
| Com- pound 2-205 | 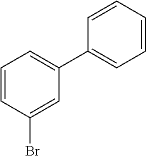 [SMILES: Brc1cccc(-c2ccccc2)c1] | | 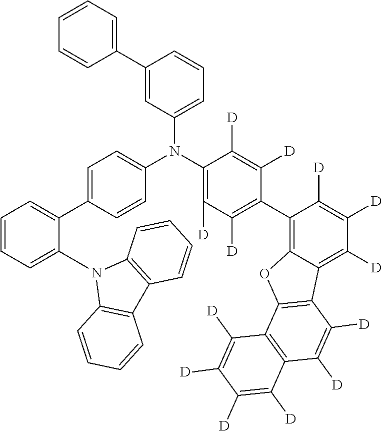 | 4.67 | 59 | 99.90 | 792.38 |
| Com- pound 2-206 | 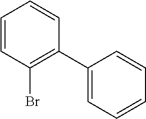 [SMILES: Brc1ccccc1-c1ccccc1] | | 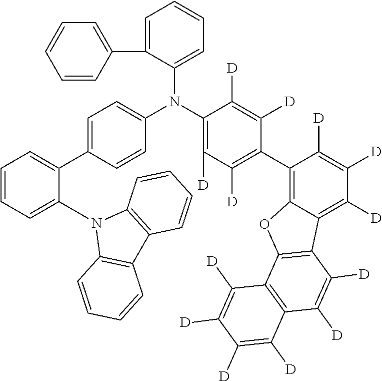 | 4.91 | 62 | 99.92 | 792.38 |
| Com- pound 2-207 | 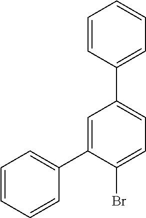 [SMILES: Brc1ccc(-c2ccccc2)cc1-c1ccccc1] | | 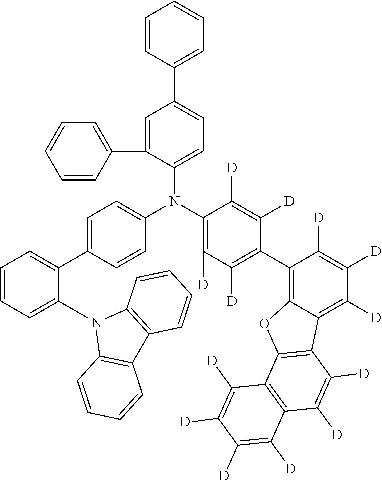 | 5.20 | 60 | 99.95 | 868.41 |
| Com- pound 2-208 | 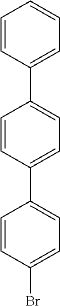 [SMILES: Brc1ccc(-c2ccc(-c3ccccc3)cc2)cc1] | | 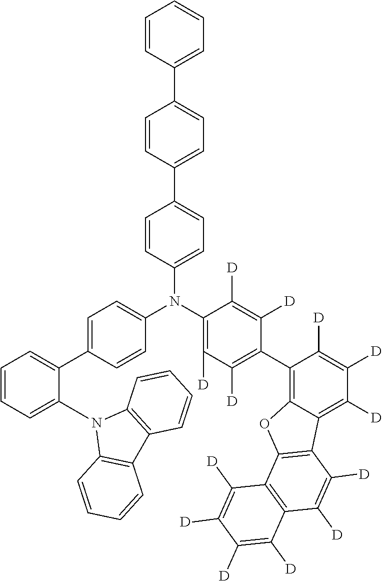 | 5.03 | 58 | 99.96 | 868.41 |
| Com- pound 2-209 | 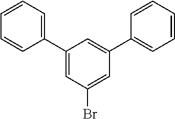 [SMILES: Brc1cc(-c2ccccc2)cc(-c2ccccc2)c1] | | 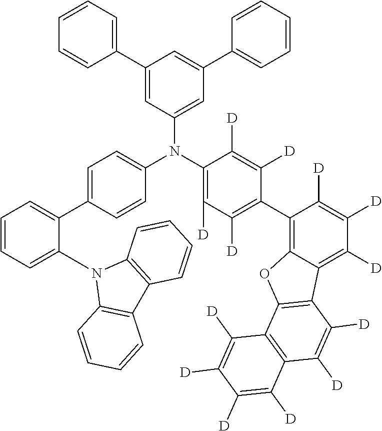 | 4.77 | 55 | 99.94 | 868.42 |
| Com- pound 2-210 | 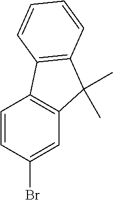 [SMILES: CC1(C)c2ccccc2-c2ccc(Br)cc21] | | 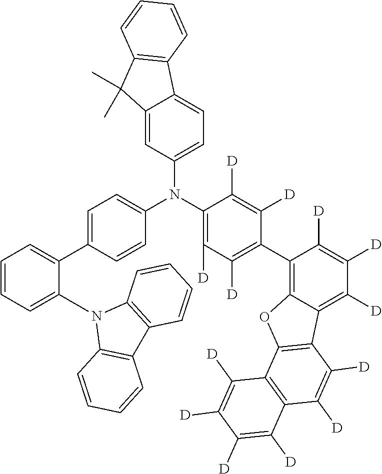 | 4.32 | 52 | 99.95 | 832.41 |
| Com- pound 2-211 | 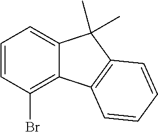 [SMILES: CC1(C)c2ccccc2-c2c(Br)cccc21] | | 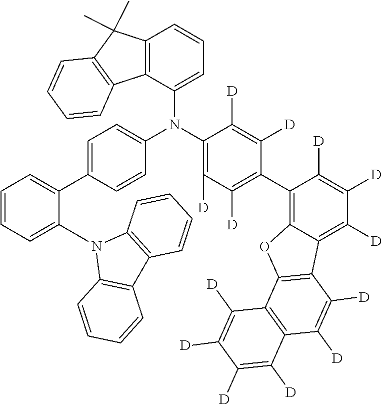 | 4.40 | 53 | 99.86 | 832.41 |
| Com- pound 2-212 | 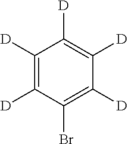 [SMILES: [2H]c1c([2H])c([2H])c(Br)c([2H])c1[2H]] | | 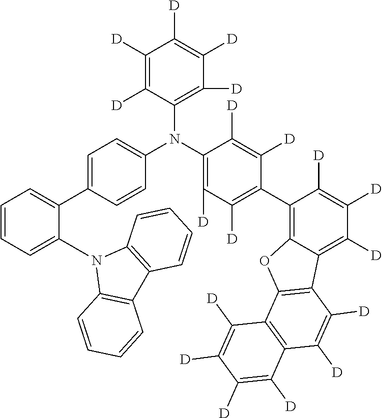 | 3.52 | 49 | 99.89 | 721.38 |
| Com- pound 2-213 | 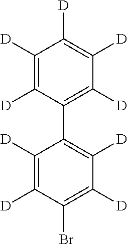 [SMILES: [2H]c1c([2H])c([2H])c(-c2c([2H])c([2H])c(Br)c([2H])c2[2H])c([2H])c1[2H]] | | 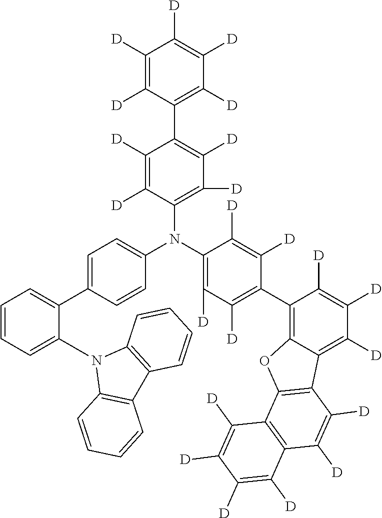 | 4.80 | 60 | 99.90 | 801.44 |
| Com- pound 2-214 | 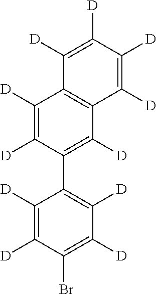 [SMILES: [2H]c1c([2H])c(-c2c([2H])c([2H])c3c([2H])c([2H])c([2H])c([2H])c3c2[2H])c([2H])c([2H])c1Br] | | 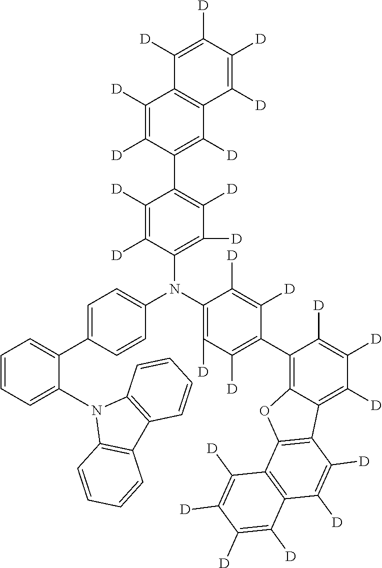 | 5.29 | 62 | 99.95 | 853.47 |

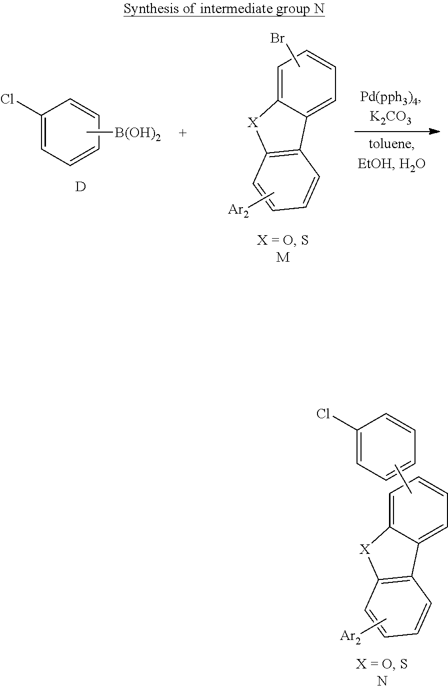

Reactant D (17.2 g, 110 mmol), reactant M (100 mmol), Pd(PPh₃)₄ (2.31 g, 2.0 mmol), K₂CO₃ (27.6 g, 200 mmol), toluene (150 mL), water (80 mL), and ethanol (50 mL) were added to a round bottom flask under nitrogen atmosphere and then stirred at 100° C. to perform reaction. When the reaction was finished, after separating an organic layer, then removing water, and performing filtering, the mixture was purified in a silica gel column and recrystallization method to manufacture intermediate group N.

The result is expressed in Table 7 below.

TABLE 7

| | | | | Acquired | | |
|---|---|---|---|---|---|---|
| | | | | amount | Yield | Purity |
| Items | Reactant D | Reactant M | Intermediate group N | (g) | (%) | (%) |
| N-1 | 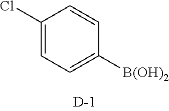 | 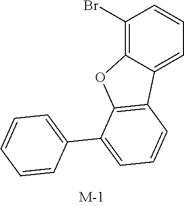 [SMILES: Brc1cccc2c1oc1c(-c3ccccc3)cccc12] | 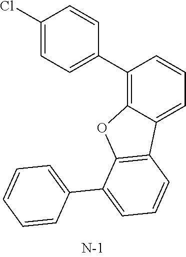 [SMILES: Clc1ccc(-c2cccc3c2oc2c(-c4ccccc4)cccc23)cc1] | 27.9 | 79 | 99.1 |
| N-2 | | 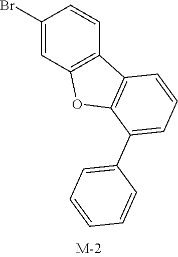 [SMILES: Brc1ccc2c(c1)oc1c(-c3ccccc3)cccc12] | 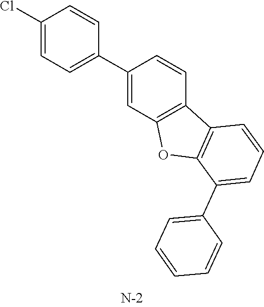 [SMILES: Clc1ccc(-c2ccc3c(c2)oc2c(-c4ccccc4)cccc23)cc1] | 26.5 | 75 | 99.2 |
| N-3 | | 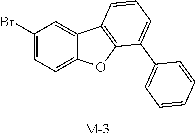 | 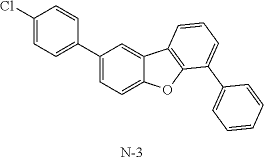 [SMILES: Clc1ccc(-c2ccc3oc4c(-c5ccccc5)cccc4c3c2)cc1] | 28.6 | 81 | 99.1 |
| N-4 | | 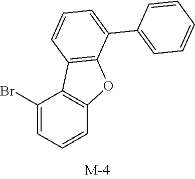 [SMILES: Brc1cccc2oc3c(-c4ccccc4)cccc3c12] | 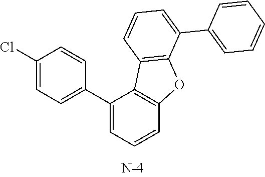 [SMILES: Clc1ccc(-c2cccc3oc4c(-c5ccccc5)cccc4c23)cc1] | 25.1 | 71 | 98.9 |
| N-5 | | 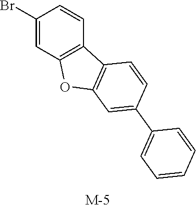 [SMILES: Brc1ccc2c(c1)oc1cc(-c3ccccc3)ccc12] | 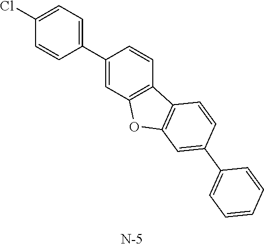 [SMILES: Clc1ccc(-c2ccc3c(c2)oc2cc(-c4ccccc4)ccc23)cc1] | 23.7 | 67 | 98.2 |
| N-6 | | 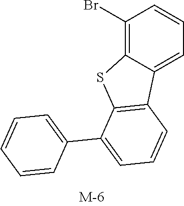 [SMILES: Brc1cccc2c1sc1c(-c3ccccc3)cccc12] | 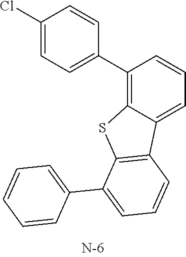 [SMILES: Clc1ccc(-c2cccc3c2sc2c(-c4ccccc4)cccc23)cc1] | 27.0 | 73 | 98.7 |
| N-7 | | 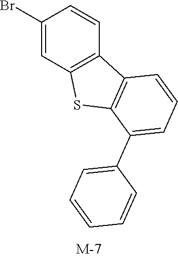 [SMILES: Brc1ccc2c(c1)sc1c(-c3ccccc3)cccc12] | 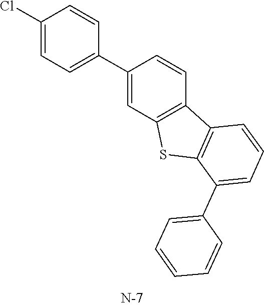 [SMILES: Clc1ccc(-c2ccc3c(c2)sc2c(-c4ccccc4)cccc23)cc1] | 25.5 | 69 | 98.6 |
| N-8 | | 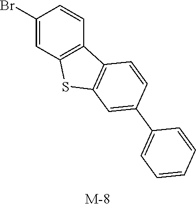 [SMILES: Brc1ccc2c(c1)sc1cc(-c3ccccc3)ccc12] | 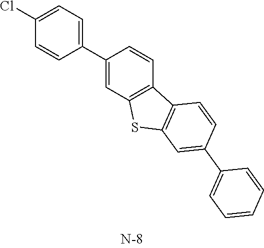 [SMILES: Clc1ccc(-c2ccc3c(c2)sc2cc(-c4ccccc4)ccc23)cc1] | 20.3 | 55 | 97.5 |
| F-9 | 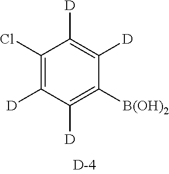 [SMILES: [2H]c1c([2H])c(B(O)O)c([2H])c([2H])c1Cl] | 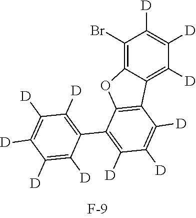 [SMILES: [2H]c1c([2H])c([2H])c(-c2c([2H])c([2H])c([2H])c3c2oc2c(Br)c([2H])c([2H])c([2H])c23)c([2H])c1[2H]] | 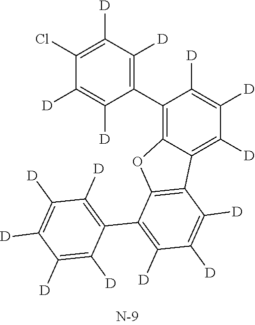 [SMILES: [2H]c1c([2H])c([2H])c(-c2c([2H])c([2H])c([2H])c3c2oc2c(-c4c([2H])c([2H])c(Cl)c([2H])c4[2H])c([2H])c([2H])c([2H])c23)c([2H])c1[2H]] | 21.7 | 59 | 98.5 |
| F-10 | | 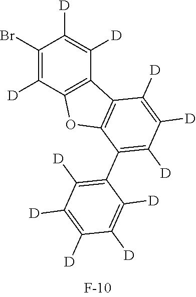 [SMILES: [2H]c1c([2H])c([2H])c(-c2c([2H])c([2H])c([2H])c3c2oc2c([2H])c(Br)c([2H])c([2H])c23)c([2H])c1[2H]] | 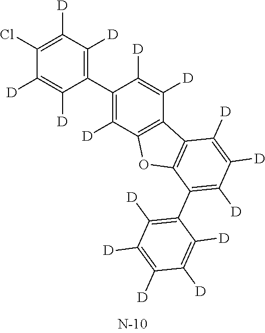 [SMILES: [2H]c1c([2H])c([2H])c(-c2c([2H])c([2H])c([2H])c3c2oc2c([2H])c(-c4c([2H])c([2H])c(Cl)c([2H])c4[2H])c([2H])c([2H])c23)c([2H])c1[2H]] | 22.8 | 62 | 97.9 |
| F-11 | | 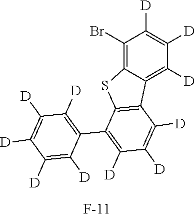 [SMILES: [2H]c1c([2H])c([2H])c(-c2c([2H])c([2H])c([2H])c3c2sc2c(Br)c([2H])c([2H])c([2H])c23)c([2H])c1[2H]] | 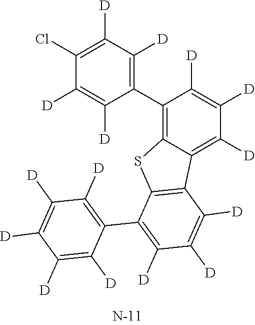 [SMILES: [2H]c1c([2H])c([2H])c(-c2c([2H])c([2H])c([2H])c3c2sc2c(-c4c([2H])c([2H])c(Cl)c([2H])c4[2H])c([2H])c([2H])c([2H])c23)c([2H])c1[2H]] | 20.0 | 52 | 98.5 |

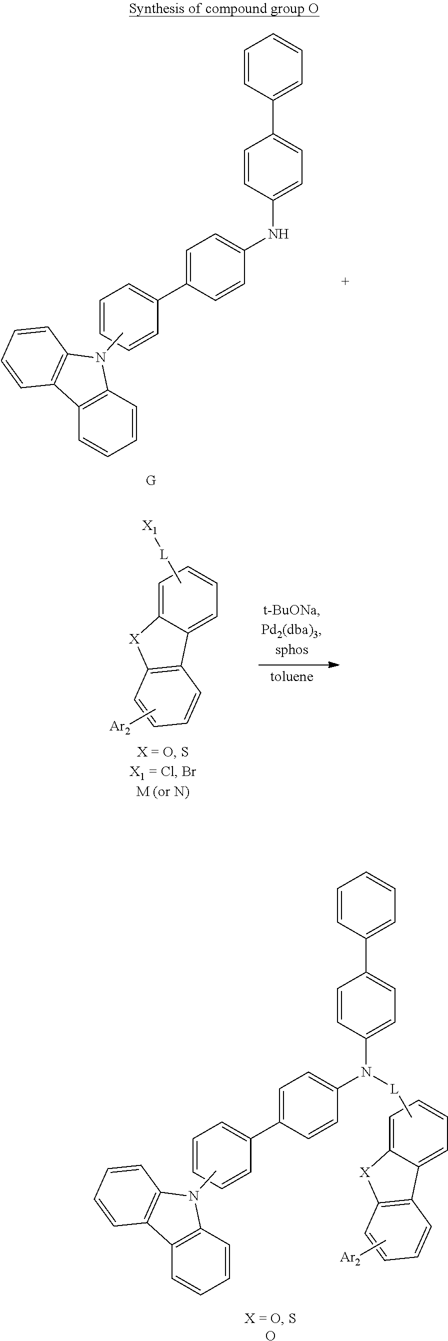

Intermediate G (4.86 g, 10 mmol), intermediate M (or N) (11 mmol), Pd₂(dba)₃ (0.183 g, 0.2 mmol), t-BuONa (1.92 g, 20 mmol), SPhos (0.164 g, 0.4 mmol), and toluene (60 mL) were added to a round bottom flask under nitrogen atmosphere and then stirred at 100° C. to perform the reaction. When the reaction was finished, the mixture was precipitated in methanol and after the precipitate was filtered, purified in a silica gel column and recrystallization method to manufacture compound group O.

The result is expressed in Table 8 below.

TABLE 8

| | | | | Acquired | | | |
|---|---|---|---|---|---|---|---|
| | | | | amount | Yield | Purity | MS |
| Items | Intermediate G | Intermediate groups M, N | Compound group O | (g) | (%) | (%) | [M + H]+ |
| Compound 1-1008 | 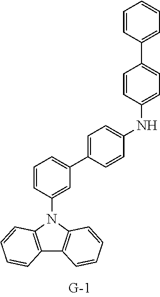 [SMILES: c1ccc(-c2ccc(Nc3ccc(-c4cccc(-n5c6ccccc6c6ccccc65)c4)cc3)cc2)cc1] | 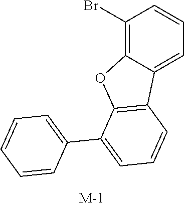 [SMILES: Brc1cccc2c1oc1c(-c3ccccc3)cccc12] | 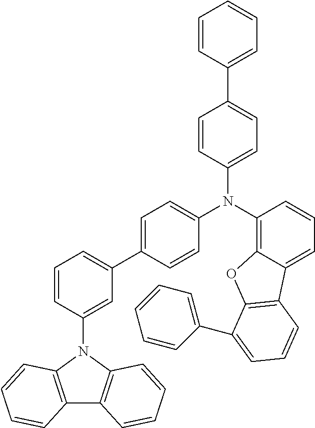 [SMILES: c1ccc(-c2ccc(N(c3ccc(-c4cccc(-n5c6ccccc6c6ccccc65)c4)cc3)c3cccc4c3oc3c(-c5ccccc5)cccc34)cc2)cc1] | 4.73 | 65 | 99.94 | 729.29 |
| Compound 1-1009 | | 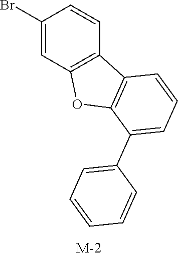 [SMILES: Brc1ccc2c(c1)oc1c(-c3ccccc3)cccc12] | 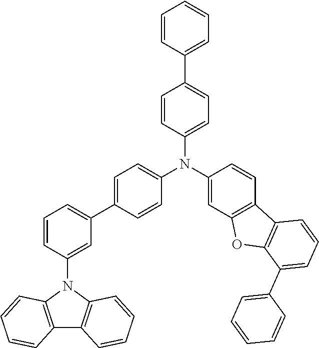 | 4.95 | 68 | 99.97 | 729.29 |
| Compound 1-1010 | | 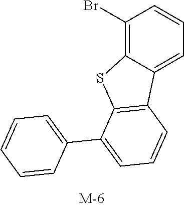 [SMILES: Brc1cccc2c1sc1c(-c3ccccc3)cccc12] | 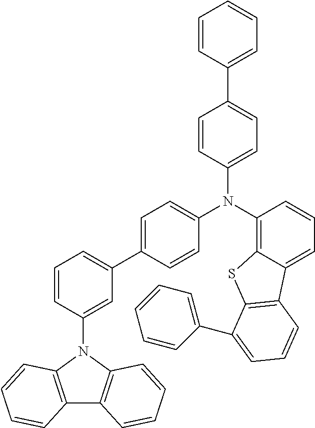 [SMILES: c1ccc(-c2ccc(N(c3ccc(-c4cccc(-n5c6ccccc6c6ccccc65)c4)cc3)c3cccc4c3sc3c(-c5ccccc5)cccc34)cc2)cc1] | 4.39 | 59 | 99.93 | 745.26 |
| Compound 1-1011 | | 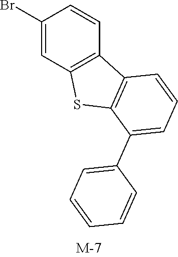 [SMILES: Brc1ccc2c(c1)sc1c(-c3ccccc3)cccc12] | 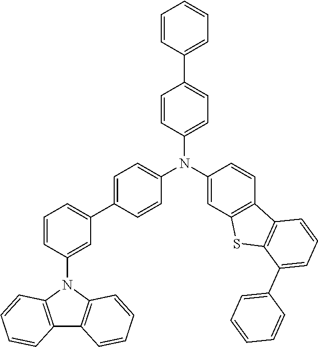 | 5.13 | 69 | 99.94 | 745.27 |
| Compound 1-1012 | | 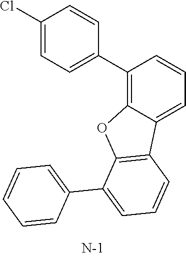 [SMILES: Clc1ccc(-c2cccc3c2oc2c(-c4ccccc4)cccc23)cc1] | 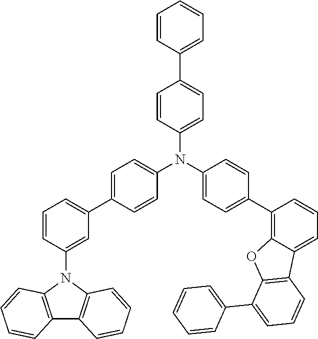 | 5.79 | 72 | 99.95 | 805.32 |
| Compound 1-1013 | | 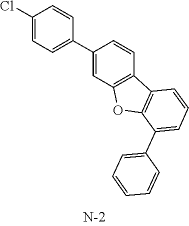 [SMILES: Clc1ccc(-c2ccc3c(c2)oc2c(-c4ccccc4)cccc23)cc1] | 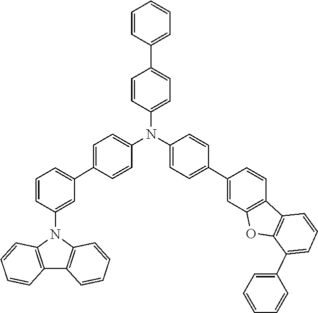 [SMILES: c1ccc(-c2ccc(N(c3ccc(-c4cccc(-n5c6ccccc6c6ccccc65)c4)cc3)c3ccc(-c4ccc5c(c4)oc4c(-c6ccccc6)cccc45)cc3)cc2)cc1] | 5.63 | 70 | 99.93 | 805.32 |
| Compound 1-1014 | | 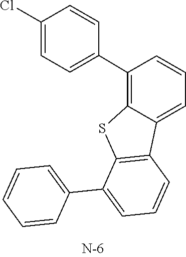 [SMILES: Clc1ccc(-c2cccc3c2sc2c(-c4ccccc4)cccc23)cc1] | 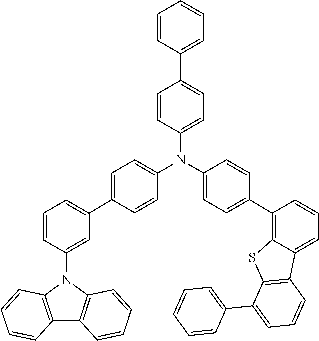 [SMILES: c1ccc(-c2ccc(N(c3ccc(-c4cccc(-n5c6ccccc6c6ccccc65)c4)cc3)c3ccc(-c4cccc5c4sc4c(-c6ccccc6)cccc45)cc3)cc2)cc1] | 5.25 | 64 | 99.92 | 821.29 |
| Compound 1-1015 | | 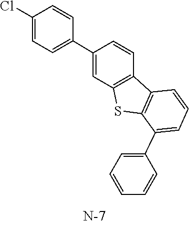 [SMILES: Clc1ccc(-c2ccc3c(c2)sc2c(-c4ccccc4)cccc23)cc1] | 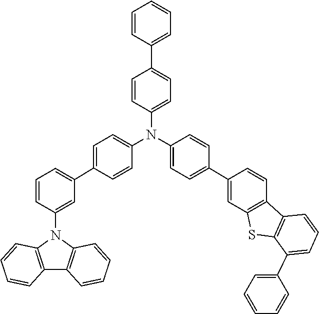 [SMILES: c1ccc(-c2ccc(N(c3ccc(-c4cccc(-n5c6ccccc6c6ccccc65)c4)cc3)c3ccc(-c4ccc5c(c4)sc4c(-c6ccccc6)cccc45)cc3)cc2)cc1] | 4.84 | 59 | 99.91 | 821.29 |
| Compound 1-1016 | | 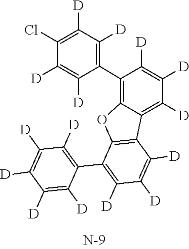 [SMILES: [2H]c1c([2H])c([2H])c(-c2c([2H])c([2H])c([2H])c3c2oc2c(-c4c([2H])c([2H])c(Cl)c([2H])c4[2H])c([2H])c([2H])c([2H])c23)c([2H])c1[2H]] | 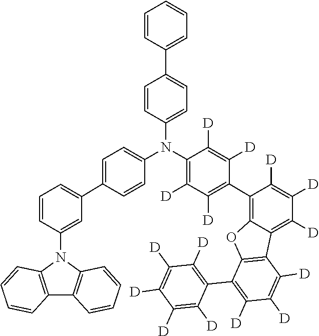 [SMILES: [2H]c1c([2H])c([2H])c(-c2c([2H])c([2H])c([2H])c3c2oc2c(-c4c([2H])c([2H])c(N(c5ccc(-c6ccccc6)cc5)c5ccc(-c6cccc(-n7c8ccccc8c8ccccc87)c6)cc5)c([2H])c4[2H])c([2H])c([2H])c([2H])c23)c([2H])c1[2H]] | 5.74 | 70 | 99.96 | 820.41 |
| Compound 1-1017 | | 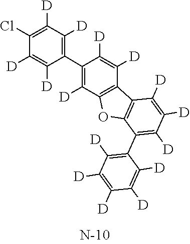 [SMILES: [2H]c1c([2H])c([2H])c(-c2c([2H])c([2H])c([2H])c3c2oc2c([2H])c(-c4c([2H])c([2H])c(Cl)c([2H])c4[2H])c([2H])c([2H])c23)c([2H])c1[2H]] | 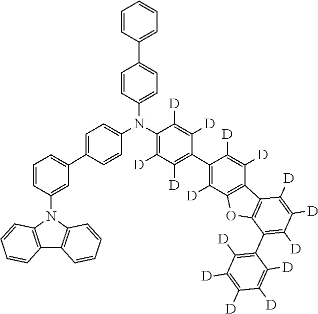 [SMILES: [2H]c1c([2H])c([2H])c(-c2c([2H])c([2H])c([2H])c3c2oc2c([2H])c(-c4c([2H])c([2H])c(N(c5ccc(-c6ccccc6)cc5)c5ccc(-c6cccc(-n7c8ccccc8c8ccccc87)c6)cc5)c([2H])c4[2H])c([2H])c([2H])c23)c([2H])c1[2H]] | 4.26 | 52 | 99.95 | 820.41 |
| Compound 1-1018 | | 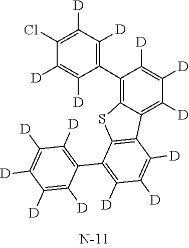 [SMILES: [2H]c1c([2H])c([2H])c(-c2c([2H])c([2H])c([2H])c3c2sc2c(-c4c([2H])c([2H])c(Cl)c([2H])c4[2H])c([2H])c([2H])c([2H])c23)c([2H])c1[2H]] | 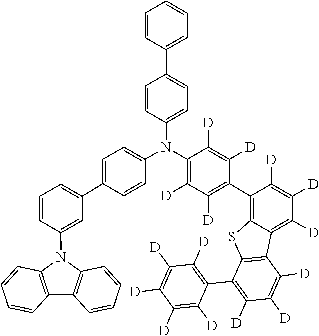 [SMILES: [2H]c1c([2H])c([2H])c(-c2c([2H])c([2H])c([2H])c3c2sc2c(-c4c([2H])c([2H])c(N(c5ccc(-c6ccccc6)cc5)c5ccc(-c6cccc(-n7c8ccccc8c8ccccc87)c6)cc5)c([2H])c4[2H])c([2H])c([2H])c([2H])c23)c([2H])c1[2H]] | 5.43 | 65 | 99.90 | 836.39 |
| Compound 2-215 | 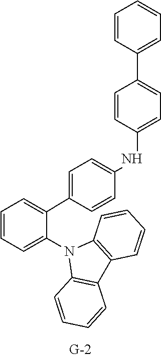 [SMILES: c1ccc(-c2ccc(Nc3ccc(-c4ccccc4-n4c5ccccc5c5ccccc54)cc3)cc2)cc1] | 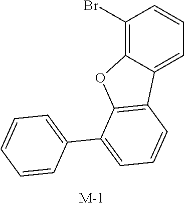 [SMILES: Brc1cccc2c1oc1c(-c3ccccc3)cccc12] | 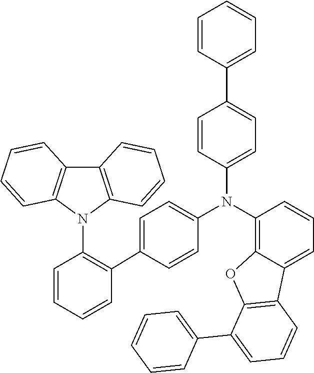 [SMILES: c1ccc(-c2ccc(N(c3ccc(-c4ccccc4-n4c5ccccc5c5ccccc54)cc3)c3cccc4c3oc3c(-c5ccccc5)cccc34)cc2)cc1] | 5.46 | 75 | 99.95 | 729.29 |
| Compound 2-216 | | 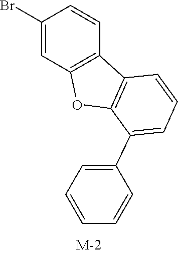 [SMILES: Brc1ccc2c(c1)oc1c(-c3ccccc3)cccc12] | 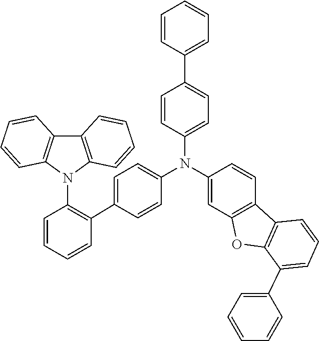 [SMILES: c1ccc(-c2ccc(N(c3ccc(-c4ccccc4-n4c5ccccc5c5ccccc54)cc3)c3ccc4c(c3)oc3c(-c5ccccc5)cccc34)cc2)cc1] | 5.54 | 76 | 99.96 | 729.28 |
| Compound 2-217 | | 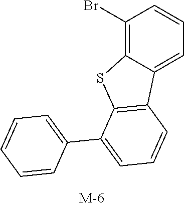 [SMILES: Brc1cccc2c1sc1c(-c3ccccc3)cccc12] | 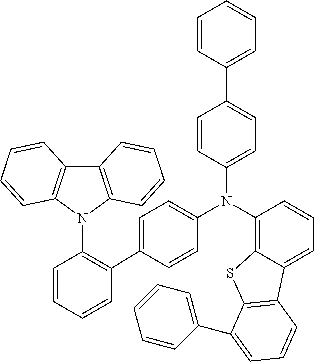 [SMILES: c1ccc(-c2ccc(N(c3ccc(-c4ccccc4-n4c5ccccc5c5ccccc54)cc3)c3cccc4c3sc3c(-c5ccccc5)cccc34)cc2)cc1] | 4.54 | 61 | 99.93 | 745.27 |
| Compound 2-218 | | 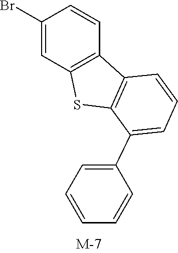 [SMILES: Brc1ccc2c(c1)sc1c(-c3ccccc3)cccc12] | 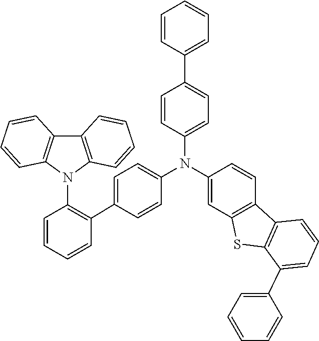 [SMILES: c1ccc(-c2ccc(N(c3ccc(-c4ccccc4-n4c5ccccc5c5ccccc54)cc3)c3ccc4c(c3)sc3c(-c5ccccc5)cccc34)cc2)cc1] | 5.80 | 78 | 99.97 | 745.26 |
| Compound 2-219 | | 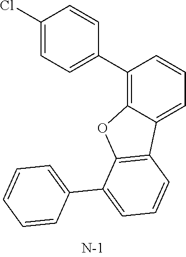 [SMILES: Clc1ccc(-c2cccc3c2oc2c(-c4ccccc4)cccc23)cc1] | 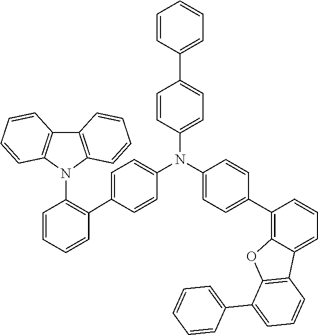 [SMILES: c1ccc(-c2ccc(N(c3ccc(-c4ccccc4-n4c5ccccc5c5ccccc54)cc3)c3ccc(-c4cccc5c4oc4c(-c6ccccc6)cccc45)cc3)cc2)cc1] | 5.79 | 72 | 99.96 | 805.32 |
| Compound 2-220 | | 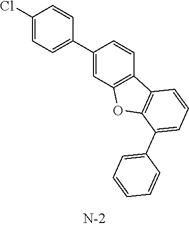 [SMILES: Clc1ccc(-c2ccc3c(c2)oc2c(-c4ccccc4)cccc23)cc1] | 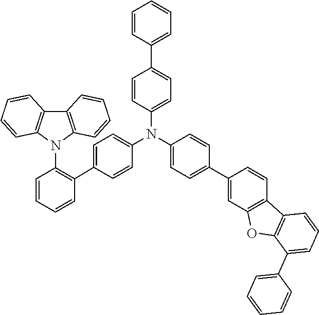 [SMILES: c1ccc(-c2ccc(N(c3ccc(-c4ccc5c(c4)oc4c(-c6ccccc6)cccc45)cc3)c3ccc(-c4ccccc4-n4c5ccccc5c5ccccc54)cc3)cc2)cc1] | 5.23 | 65 | 99.93 | 805.32 |
| Compound 2-221 | | 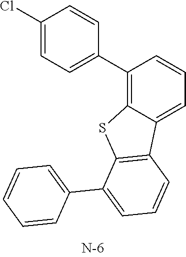 [SMILES: Clc1ccc(-c2cccc3c2sc2c(-c4ccccc4)cccc23)cc1] | 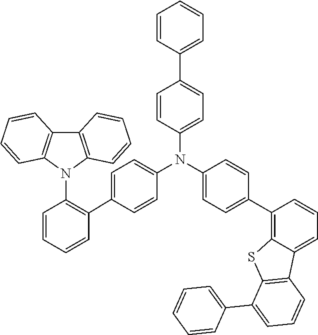 [SMILES: c1ccc(-c2ccc(N(c3ccc(-c4ccccc4-n4c5ccccc5c5ccccc54)cc3)c3ccc(-c4cccc5c4sc4c(-c6ccccc6)cccc45)cc3)cc2)cc1] | 5.00 | 61 | 99.91 | 821.29 |
| Compound 2-222 | | 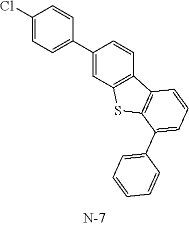 [SMILES: Clc1ccc(-c2ccc3c(c2)sc2c(-c4ccccc4)cccc23)cc1] | 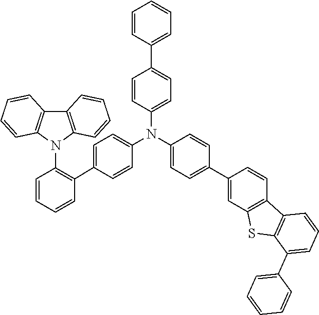 [SMILES: c1ccc(-c2ccc(N(c3ccc(-c4ccc5c(c4)sc4c(-c6ccccc6)cccc45)cc3)c3ccc(-c4ccccc4-n4c5ccccc5c5ccccc54)cc3)cc2)cc1] | 4.93 | 60 | 99.94 | 821.30 |
| Compound 2-223 | | 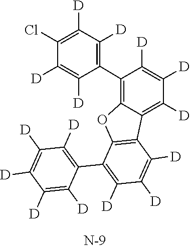 [SMILES: [2H]c1c([2H])c([2H])c(-c2c([2H])c([2H])c([2H])c3c2oc2c(-c4c([2H])c([2H])c(Cl)c([2H])c4[2H])c([2H])c([2H])c([2H])c23)c([2H])c1[2H]] | 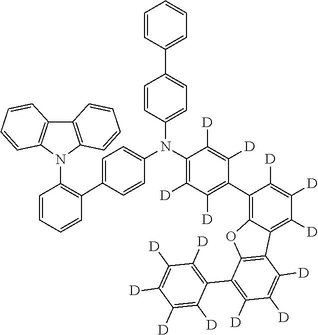 [SMILES: [2H]c1c([2H])c([2H])c(-c2c([2H])c([2H])c([2H])c3c2oc2c(-c4c([2H])c([2H])c(N(c5ccc(-c6ccccc6)cc5)c5ccc(-c6ccccc6-n6c7ccccc7c7ccccc76)cc5)c([2H])c4[2H])c([2H])c([2H])c([2H])c23)c([2H])c1[2H]] | 5.65 | 69 | 99.96 | 820.42 |
| Compound 2-224 | | 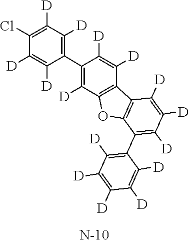 [SMILES: [2H]c1c([2H])c([2H])c(-c2c([2H])c([2H])c([2H])c3c2oc2c([2H])c(-c4c([2H])c([2H])c(Cl)c([2H])c4[2H])c([2H])c([2H])c23)c([2H])c1[2H]] | 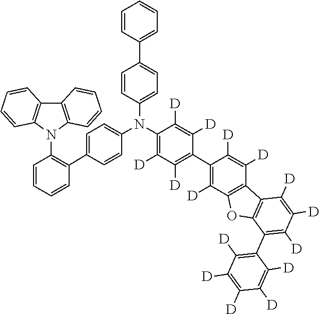 [SMILES: [2H]c1c([2H])c([2H])c(-c2c([2H])c([2H])c([2H])c3c2oc2c([2H])c(-c4c([2H])c([2H])c(N(c5ccc(-c6ccccc6)cc5)c5ccc(-c6ccccc6-n6c7ccccc7c7ccccc76)cc5)c([2H])c4[2H])c([2H])c([2H])c23)c([2H])c1[2H]] | 5.41 | 66 | 99.92 | 820.41 |
| Compound 2-225 | | 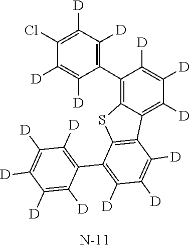 [SMILES: [2H]c1c([2H])c([2H])c(-c2c([2H])c([2H])c([2H])c3c2sc2c(-c4c([2H])c([2H])c(Cl)c([2H])c4[2H])c([2H])c([2H])c([2H])c23)c([2H])c1[2H]] | 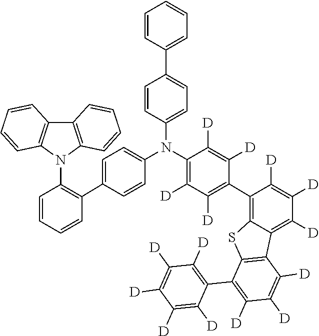 [SMILES: [2H]c1c([2H])c([2H])c(-c2c([2H])c([2H])c([2H])c3c2sc2c(-c4c([2H])c([2H])c(N(c5ccc(-c6ccccc6)cc5)c5ccc(-c6ccccc6-n6c7ccccc7c7ccccc76)cc5)c([2H])c4[2H])c([2H])c([2H])c([2H])c23)c([2H])c1[2H]] | 5.68 | 68 | 99.90 | 836.39 |

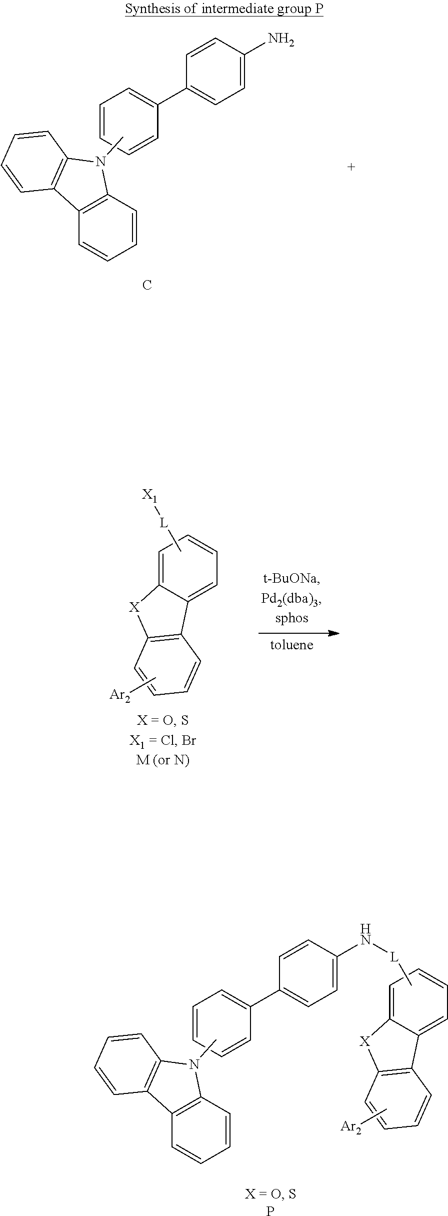

Intermediate C (33.4 g, 100 mmol), intermediate M (or N) (110 mmol), Pd₂(dha)₃ (1.83 g, 2.0 mmol), t-BuONa (19.2 g, 200 mmol), SPhos (1.64 g, 4.0 mmol), and toluene (560 mL) were added to a round bottom flask under nitrogen atmosphere and then stirred at 100° C. to perform the reaction. When the reaction was finished, the mixture was precipitated in methanol and after the precipitate was filtered, purified in a silica gel column and recrystallization method to manufacture intermediate group P.

The result is expressed in Table 9 below.

TABLE 9

| | | | | Acquired | | |
|---|---|---|---|---|---|---|
| | | | | amount | Yield | Purity |
| Items | Intermediate group C | Intermediate groups M, N | Intermediate group P | (g) | (%) | (%) |
| P-1 | 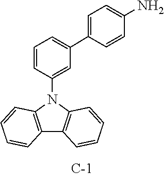 [SMILES: Nc1ccc(-c2cccc(-n3c4ccccc4c4ccccc43)c2)cc1] | 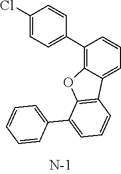 [SMILES: Clc1ccc(-c2cccc3c2oc2c(-c4ccccc4)cccc23)cc1] | 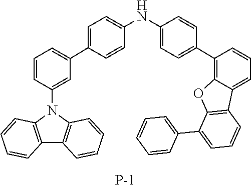 [SMILES: c1ccc(-c2cccc3c2oc2c(-c4ccc(Nc5ccc(-c6cccc(-n7c8ccccc8c8ccccc87)c6)cc5)cc4)cccc23)cc1] | 41.7 | 64 | 98.3 |
| P-2 | | 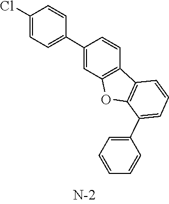 [SMILES: Clc1ccc(-c2ccc3c(c2)oc2c(-c4ccccc4)cccc23)cc1] | 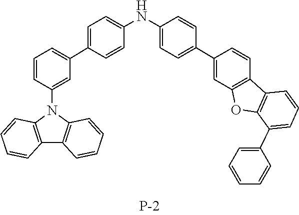 [SMILES: c1ccc(-c2cccc3c2oc2cc(-c4ccc(Nc5ccc(-c6cccc(-n7c8ccccc8c8ccccc87)c6)cc5)cc4)ccc23)cc1] | 46.2 | 71 | 98.2 |
| P-3 | | 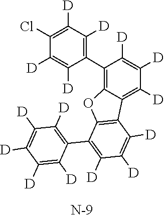 [SMILES: [2H]c1c([2H])c([2H])c(-c2c([2H])c([2H])c([2H])c3c2oc2c(-c4c([2H])c([2H])c(Cl)c([2H])c4[2H])c([2H])c([2H])c([2H])c23)c([2H])c1[2H]] | 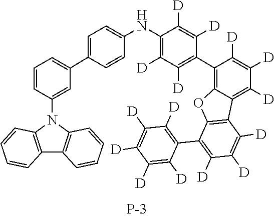 [SMILES: [2H]c1c([2H])c([2H])c(-c2c([2H])c([2H])c([2H])c3c2oc2c(-c4c([2H])c([2H])c(Nc5ccc(-c6cccc(-n7c8ccccc8c8ccccc87)c6)cc5)c([2H])c4[2H])c([2H])c([2H])c([2H])c23)c([2H])c1[2H]] | 39.3 | 59 | 98.8 |
| P-4 | | 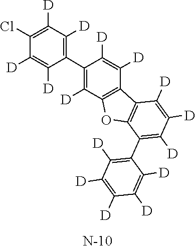 [SMILES: [2H]c1c([2H])c([2H])c(-c2c([2H])c([2H])c([2H])c3c2oc2c([2H])c(-c4c([2H])c([2H])c(Cl)c([2H])c4[2H])c([2H])c([2H])c23)c([2H])c1[2H]] | 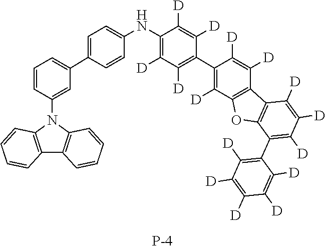 [SMILES: [2H]c1c([2H])c([2H])c(-c2c([2H])c([2H])c([2H])c3c2oc2c([2H])c(-c4c([2H])c([2H])c(Nc5ccc(-c6cccc(-n7c8ccccc8c8ccccc87)c6)cc5)c([2H])c4[2H])c([2H])c([2H])c23)c([2H])c1[2H]] | 48.0 | 72 | 98.4 |
| P-5 | 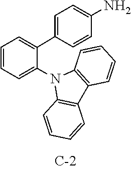 [SMILES: Nc1ccc(-c2ccccc2-n2c3ccccc3c3ccccc32)cc1] | 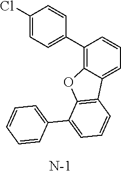 [SMILES: Clc1ccc(-c2cccc3c2oc2c(-c4ccccc4)cccc23)cc1] | 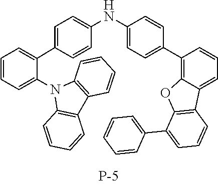 [SMILES: c1ccc(-c2cccc3c2oc2c(-c4ccc(Nc5ccc(-c6ccccc6-n6c7ccccc7c7ccccc76)cc5)cc4)cccc23)cc1] | 47.5 | 73 | 99.2 |
| P-6 | | 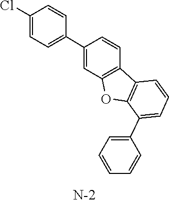 [SMILES: Clc1ccc(-c2ccc3c(c2)oc2c(-c4ccccc4)cccc23)cc1] | 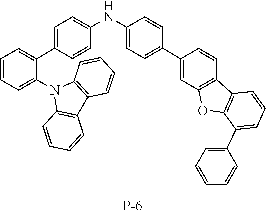 [SMILES: c1ccc(-c2cccc3c2oc2cc(-c4ccc(Nc5ccc(-c6ccccc6-n6c7ccccc7c7ccccc76)cc5)cc4)ccc23)cc1] | 44.9 | 69 | 98.5 |
| P-7 | | 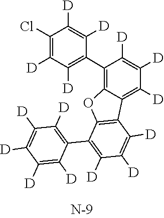 [SMILES: [2H]c1c([2H])c([2H])c(-c2c([2H])c([2H])c([2H])c3c2oc2c(-c4c([2H])c([2H])c(Cl)c([2H])c4[2H])c([2H])c([2H])c([2H])c23)c([2H])c1[2H]] | 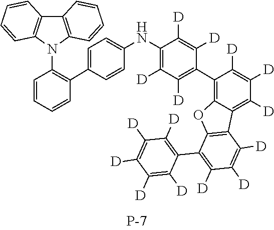 [SMILES: [2H]c1c([2H])c([2H])c(-c2c([2H])c([2H])c([2H])c3c2oc2c(-c4c([2H])c([2H])c(Nc5ccc(-c6ccccc6-n6c7ccccc7c7ccccc76)cc5)c([2H])c4[2H])c([2H])c([2H])c([2H])c23)c([2H])c1[2H]] | 45.3 | 68 | 98.2 |
| P-8 | | 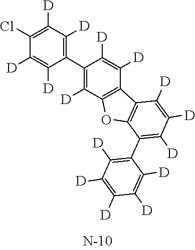 [SMILES: [2H]c1c([2H])c([2H])c(-c2c([2H])c([2H])c([2H])c3c2oc2c([2H])c(-c4c([2H])c([2H])c(Cl)c([2H])c4[2H])c([2H])c([2H])c23)c([2H])c1[2H]] | 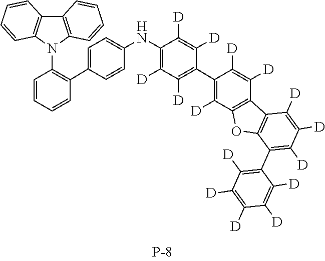 [SMILES: [2H]c1c([2H])c([2H])c(-c2c([2H])c([2H])c([2H])c3c2oc2c([2H])c(-c4c([2H])c([2H])c(Nc5ccc(-c6ccccc6-n6c7ccccc7c7ccccc76)cc5)c([2H])c4[2H])c([2H])c([2H])c23)c([2H])c1[2H]] | 44.0 | 66 | 99.0 |

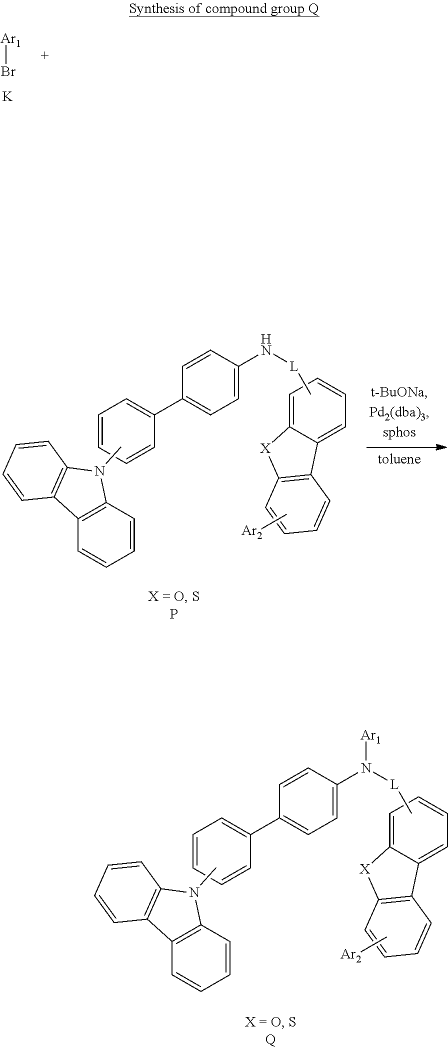

Reactant K (11 mmol), intermediate P (10 mmol), Pd₂(dba)₃ (0.183 g, 0.2 mmol), t-BuONa (1.92 g, 20 mmol), SPhos (0.164 g, 0.4 mmol), and toluene (60 mL) were added to a round bottom flask under nitrogen atmosphere and then stirred at 100° C. to perform the reaction. When the reaction was finished, the mixture was precipitated in methanol and after the precipitate was filtered, purified in a silica gel column and recrystallization method to manufacture compound group Q.

The result is expressed in Table 10 below.

TABLE 10

| Items | Reactant K | Intermediate group P |
|---|---|---|
| Compound 1-1019 | 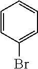 | 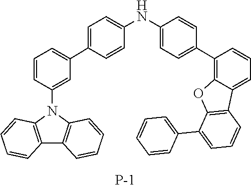 [SMILES: c1ccc(-c2cccc3c2oc2c(-c4ccc(Nc5ccc(-c6cccc(-n7c8ccccc8c8ccccc87)c6)cc5)cc4)cccc23)cc1] |
| Compound 1-1023 | 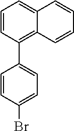 [SMILES: Brc1ccc(-c2cccc3ccccc23)cc1] | |
| Compound 1-1024 | 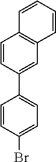 [SMILES: Brc1ccc(-c2ccc3ccccc3c2)cc1] | |
| Compound 1-1033 | 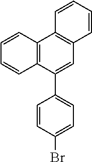 [SMILES: Brc1ccc(-c2cc3ccccc3c3ccccc23)cc1] | |
| Compound 1-1025 | 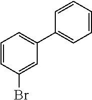 [SMILES: Brc1cccc(-c2ccccc2)c1] | |
| Compound 1-1026 | 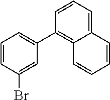 [SMILES: Brc1cccc(-c2cccc3ccccc23)c1] | |
| Compound 1-1027 | 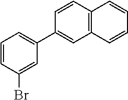 [SMILES: Brc1cccc(-c2ccc3ccccc3c2)c1] | |
| Compound 1-1029 | 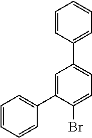 [SMILES: Brc1ccc(-c2ccccc2)cc1-c1ccccc1] | |
| Compound 1-1028 | 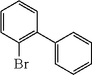 [SMILES: Brc1ccccc1-c1ccccc1] | |
| Compound 1-1030 | 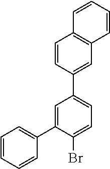 [SMILES: Brc1ccc(-c2ccc3ccccc3c2)cc1-c1ccccc1] | |
| Compound 1-1031 | 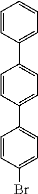 [SMILES: Brc1ccc(-c2ccc(-c3ccccc3)cc2)cc1] | |
| Compound 1-1032 | 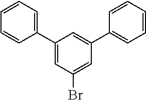 [SMILES: Brc1cc(-c2ccccc2)cc(-c2ccccc2)c1] | |
| Compound 1-1034 | 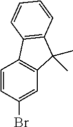 [SMILES: CC1(C)c2ccccc2-c2ccc(Br)cc21] | |
| Compound 1-1035 | 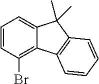 [SMILES: CC1(C)c2ccccc2-c2c(Br)cccc21] | |
| Compound 1-1036 | 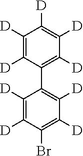 [SMILES: [2H]c1c([2H])c([2H])c(-c2c([2H])c([2H])c(Br)c([2H])c2[2H])c([2H])c1[2H]] | |
| Compound 1-1037 | 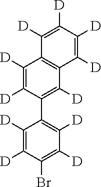 [SMILES: [2H]c1c([2H])c(-c2c([2H])c([2H])c3c([2H])c([2H])c([2H])c([2H])c3c2[2H])c([2H])c([2H])c1Br] | |
| Compound 1-1038 | 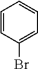 | 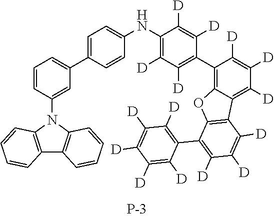 [SMILES: [2H]c1c([2H])c([2H])c(-c2c([2H])c([2H])c([2H])c3c2oc2c(-c4c([2H])c([2H])c(Nc5ccc(-c6cccc(-n7c8ccccc8c8ccccc87)c6)cc5)c([2H])c4[2H])c([2H])c([2H])c([2H])c23)c([2H])c1[2H]] |
| Compound 1-1039 | 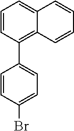 [SMILES: Brc1ccc(-c2cccc3ccccc23)cc1] | |
| Compound 1-1040 | 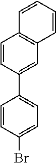 [SMILES: Brc1ccc(-c2ccc3ccccc3c2)cc1] | |
| Compound 1-1041 | 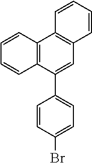 [SMILES: Brc1ccc(-c2cc3ccccc3c3ccccc23)cc1] | |
| Compound 1-1042 | 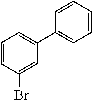 [SMILES: Brc1cccc(-c2ccccc2)c1] | |
| Compound 1-1043 | 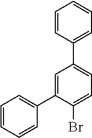 [SMILES: Brc1ccc(-c2ccccc2)cc1-c1ccccc1] | |
| Compound 1-1044 | 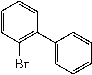 [SMILES: Brc1ccccc1-c1ccccc1] | |
| Compound 1-1045 | 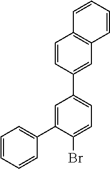 [SMILES: Brc1ccc(-c2ccc3ccccc3c2)cc1-c1ccccc1] | |
| Compound 1-1046 | 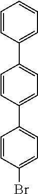 [SMILES: Brc1ccc(-c2ccc(-c3ccccc3)cc2)cc1] | |
| Compound 1-1047 | 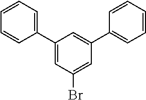 [SMILES: Brc1cc(-c2ccccc2)cc(-c2ccccc2)c1] | |
| Compound 1-1048 | 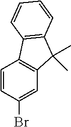 [SMILES: CC1(C)c2ccccc2-c2ccc(Br)cc21] | |
| Compound 1-1049 | 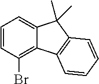 [SMILES: CC1(C)c2ccccc2-c2c(Br)cccc21] | |
| Compound 1-1050 | 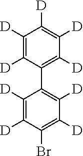 [SMILES: [2H]c1c([2H])c([2H])c(-c2c([2H])c([2H])c(Br)c([2H])c2[2H])c([2H])c1[2H]] | |
| Compound 1-1051 | 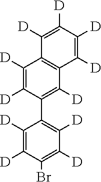 [SMILES: [2H]c1c([2H])c(-c2c([2H])c([2H])c3c([2H])c([2H])c([2H])c([2H])c3c2[2H])c([2H])c([2H])c1Br] | |
| Compound 2-226 | 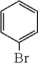 | 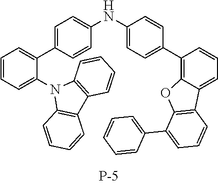 [SMILES: c1ccc(-c2cccc3c2oc2c(-c4ccc(Nc5ccc(-c6ccccc6-n6c7ccccc7c7ccccc76)cc5)cc4)cccc23)cc1] |
| Compound 2-231 | 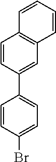 [SMILES: Brc1ccc(-c2ccc3ccccc3c2)cc1] | |
| Compound 2-232 | 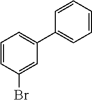 [SMILES: Brc1cccc(-c2ccccc2)c1] | |
| Compound 2-236 | 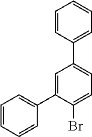 [SMILES: Brc1ccc(-c2ccccc2)cc1-c1ccccc1] | |
| Compound 2-238 | 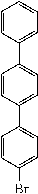 [SMILES: Brc1ccc(-c2ccc(-c3ccccc3)cc2)cc1] | |
| Compound 2-243 | 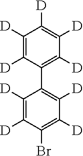 [SMILES: [2H]c1c([2H])c([2H])c(-c2c([2H])c([2H])c(Br)c([2H])c2[2H])c([2H])c1[2H]] | |
| Compound 2-244 | 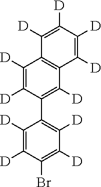 [SMILES: [2H]c1c([2H])c(-c2c([2H])c([2H])c3c([2H])c([2H])c([2H])c([2H])c3c2[2H])c([2H])c([2H])c1Br] | |
| Compound 2-245 | 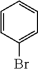 | |
| Compound 2-246 | 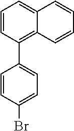 [SMILES: Brc1ccc(-c2cccc3ccccc23)cc1] | |
| Compound 2-247 | 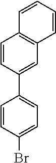 [SMILES: Brc1ccc(-c2ccc3ccccc3c2)cc1] | |
| Compound 2-248 | 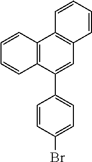 [SMILES: Brc1ccc(-c2cc3ccccc3c3ccccc23)cc1] | |
| Compound 2-249 | 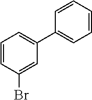 [SMILES: Brc1cccc(-c2ccccc2)c1] | |
| Compound 2-253 | 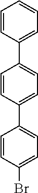 [SMILES: Brc1ccc(-c2ccc(-c3ccccc3)cc2)cc1] | |
| Compound 2-254 | 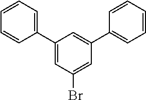 [SMILES: Brc1cc(-c2ccccc2)cc(-c2ccccc2)c1] | |
| Compound 2-255 | 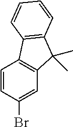 [SMILES: CC1(C)c2ccccc2-c2ccc(Br)cc21] | |
| Compound 2-257 | 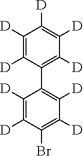 [SMILES: [2H]c1c([2H])c([2H])c(-c2c([2H])c([2H])c(Br)c([2H])c2[2H])c([2H])c1[2H]] | |
| Compound 2-258 | 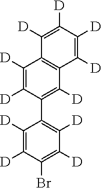 [SMILES: [2H]c1c([2H])c(-c2c([2H])c([2H])c3c([2H])c([2H])c([2H])c([2H])c3c2[2H])c([2H])c([2H])c1Br] | |

| | | Acquired | | | |
|---|---|---|---|---|---|
| | | amount | Yield | Purity | MS |
| | Compound group Q | (g) | (%) | (%) | [M + H]+ |
| Compound 1-1019 | 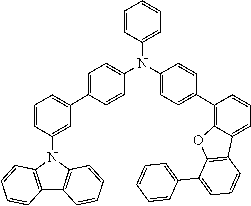 [SMILES: c1ccc(-c2cccc3c2oc2c(-c4ccc(N(c5ccccc5)c5ccc(-c6cccc(-n7c8ccccc8c8ccccc87)c6)cc5)cc4)cccc23)cc1] | 3.78 | 52 | 99.95 | 729.29 |
| Compound 1-1023 | 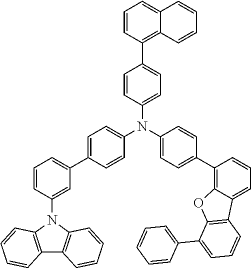 [SMILES: c1ccc(-c2cccc3c2oc2c(-c4ccc(N(c5ccc(-c6cccc(-n7c8ccccc8c8ccccc87)c6)cc5)c5ccc(-c6cccc7ccccc67)cc5)cc4)cccc23)cc1] | 4.10 | 48 | 99.92 | 855.32 |
| Compound 1-1024 | 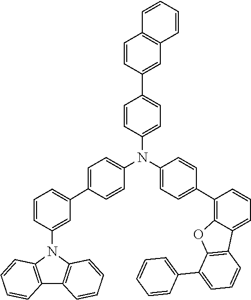 [SMILES: c1ccc(-c2cccc3c2oc2c(-c4ccc(N(c5ccc(-c6cccc(-n7c8ccccc8c8ccccc87)c6)cc5)c5ccc(-c6ccc7ccccc7c6)cc5)cc4)cccc23)cc1] | 4.78 | 56 | 99.95 | 855.33 |
| Compound 1-1033 | 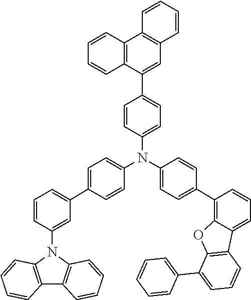 [SMILES: c1ccc(-c2cccc3c2oc2c(-c4ccc(N(c5ccc(-c6cccc(-n7c8ccccc8c8ccccc87)c6)cc5)c5ccc(-c6cc7ccccc7c7ccccc67)cc5)cc4)cccc23)cc1] | 5.43 | 60 | 99.90 | 905.36 |
| Compound 1-1025 | 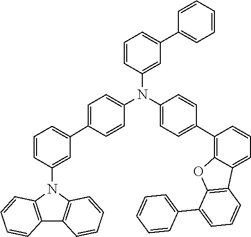 [SMILES: c1ccc(-c2cccc(N(c3ccc(-c4cccc(-n5c6ccccc6c6ccccc65)c4)cc3)c3ccc(-c4cccc5c4oc4c(-c6ccccc6)cccc45)cc3)c2)cc1] | 4.26 | 53 | 99.96 | 805.32 |
| Compound 1-1026 | 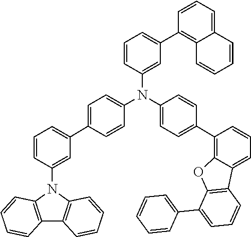 [SMILES: c1ccc(-c2cccc3c2oc2c(-c4ccc(N(c5ccc(-c6cccc(-n7c8ccccc8c8ccccc87)c6)cc5)c5cccc(-c6cccc7ccccc67)c5)cc4)cccc23)cc1] | 4.78 | 56 | 99.95 | 855.33 |
| Compound 1-1027 | 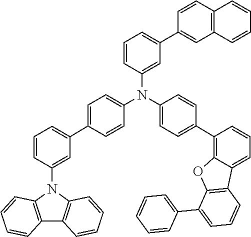 [SMILES: c1ccc(-c2cccc3c2oc2c(-c4ccc(N(c5ccc(-c6cccc(-n7c8ccccc8c8ccccc87)c6)cc5)c5cccc(-c6ccc7ccccc7c6)c5)cc4)cccc23)cc1] | 4.61 | 54 | 99.90 | 855.33 |
| Compound 1-1029 | 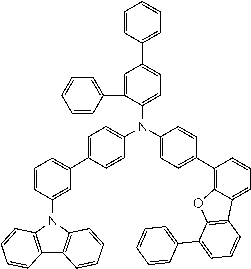 [SMILES: c1ccc(-c2ccc(N(c3ccc(-c4cccc(-n5c6ccccc6c6ccccc65)c4)cc3)c3ccc(-c4cccc5c4oc4c(-c6ccccc6)cccc45)cc3)c(-c3ccccc3)c2)cc1] | 4.31 | 49 | 99.94 | 881.35 |
| Compound 1-1028 | 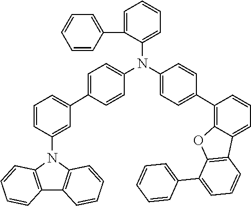 [SMILES: c1ccc(-c2ccccc2N(c2ccc(-c3cccc(-n4c5ccccc5c5ccccc54)c3)cc2)c2ccc(-c3cccc4c3oc3c(-c5ccccc5)cccc34)cc2)cc1] | 4.50 | 56 | 99.95 | 805.32 |
| Compound 1-1030 | 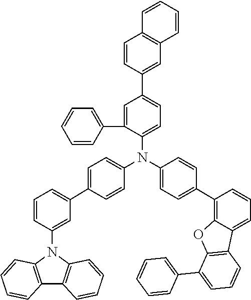 [SMILES: c1ccc(-c2cc(-c3ccc4ccccc4c3)ccc2N(c2ccc(-c3cccc(-n4c5ccccc5c5ccccc54)c3)cc2)c2ccc(-c3cccc4c3oc3c(-c5ccccc5)cccc34)cc2)cc1] | 5.39 | 58 | 99.96 | 931.36 |
| Compound 1-1031 | 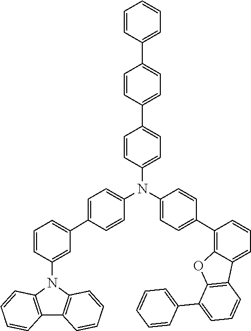 [SMILES: c1ccc(-c2ccc(-c3ccc(N(c4ccc(-c5cccc(-n6c7ccccc7c7ccccc76)c5)cc4)c4ccc(-c5cccc6c5oc5c(-c7ccccc7)cccc56)cc4)cc3)cc2)cc1] | 5.02 | 57 | 99.98 | 881.35 |
| Compound 1-1032 | 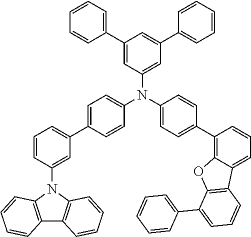 [SMILES: c1ccc(-c2cc(-c3ccccc3)cc(N(c3ccc(-c4cccc(-n5c6ccccc6c6ccccc65)c4)cc3)c3ccc(-c4cccc5c4oc4c(-c6ccccc6)cccc45)cc3)c2)cc1] | 4.93 | 56 | 99.92 | 881.36 |
| Compound 1-1034 | 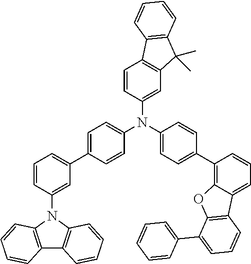 [SMILES: CC1(C)c2ccccc2-c2ccc(N(c3ccc(-c4cccc(-n5c6ccccc6c6ccccc65)c4)cc3)c3ccc(-c4cccc5c4oc4c(-c6ccccc6)cccc45)cc3)cc21] | 4.81 | 57 | 99.92 | 845.35 |
| Compound 1-1035 | 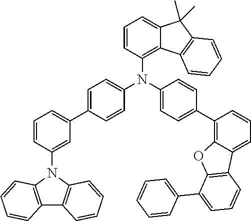 [SMILES: CC1(C)c2ccccc2-c2c(N(c3ccc(-c4cccc(-n5c6ccccc6c6ccccc65)c4)cc3)c3ccc(-c4cccc5c4oc4c(-c6ccccc6)cccc45)cc3)cccc21] | 5.40 | 64 | 99.90 | 845.35 |
| Compound 1-1036 | 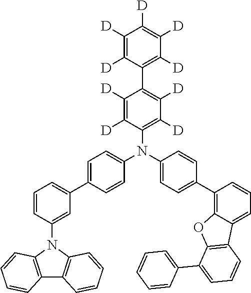 [SMILES: [2H]c1c([2H])c([2H])c(-c2c([2H])c([2H])c(N(c3ccc(-c4cccc(-n5c6ccccc6c6ccccc65)c4)cc3)c3ccc(-c4cccc5c4oc4c(-c6ccccc6)cccc45)cc3)c([2H])c2[2H])c([2H])c1[2H]] | 5.53 | 68 | 99.94 | 814.37 |
| Compound 1-1037 | 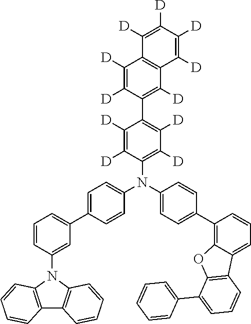 [SMILES: [2H]c1c([2H])c(N(c2ccc(-c3cccc(-n4c5ccccc5c5ccccc54)c3)cc2)c2ccc(-c3cccc4c3oc3c(-c5ccccc5)cccc34)cc2)c([2H])c([2H])c1-c1c([2H])c([2H])c2c([2H])c([2H])c([2H])c([2H])c2c1[2H]] | 4.33 | 50 | 99.94 | 866.40 |
| Compound 1-1038 | 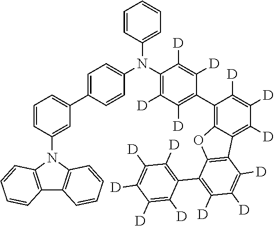 [SMILES: [2H]c1c([2H])c([2H])c(-c2c([2H])c([2H])c([2H])c3c2oc2c(-c4c([2H])c([2H])c(N(c5ccccc5)c5ccc(-c6cccc(-n7c8ccccc8c8ccccc87)c6)cc5)c([2H])c4[2H])c([2H])c([2H])c([2H])c23)c([2H])c1[2H]] | 4.31 | 58 | 99.92 | 744.38 |
| Compound 1-1039 | 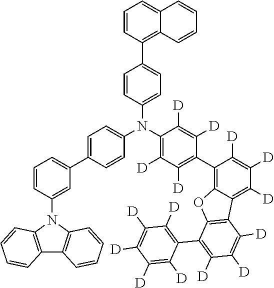 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5ccc(-c6cccc7ccccc67)cc5)c([2H])c4[2H])c([2H])c([2H])c([2H])c23)c([2H])c1[2H]] | 5.82 | 67 | 99.97 | 870.43 |
| Compound 1-1040 | 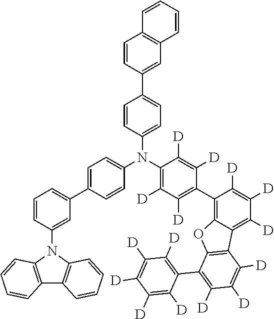 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5ccc(-c6ccc7ccccc7c6)cc5)c([2H])c4[2H])c([2H])c([2H])c([2H])c23)c([2H])c1[2H]] | 5.13 | 59 | 99.96 | 870.43 |
| Compound 1-1041 | 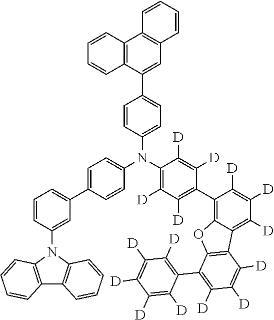 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5ccc(-c6cc7ccccc7c7ccccc67)cc5)c([2H])c4[2H])c([2H])c([2H])c([2H])c23)c([2H])c1[2H]] | 4.87 | 53 | 99.95 | 920.44 |
| Compound 1-1042 | 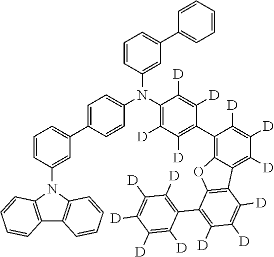 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5cccc(-c6ccccc6)c5)c([2H])c4[2H])c([2H])c([2H])c([2H])c23)c([2H])c1[2H]] | 5.08 | 62 | 99.90 | 820.40 |
| Compound 1-1043 | 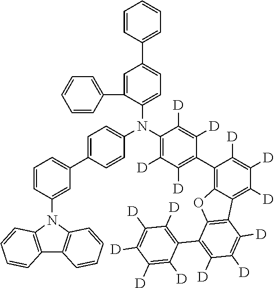 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5ccc(-c6ccccc6)cc5-c5ccccc5)c([2H])c4[2H])c([2H])c([2H])c([2H])c23)c([2H])c1[2H]] | 4.83 | 54 | 99.96 | 896.44 |
| Compound 1-1044 | 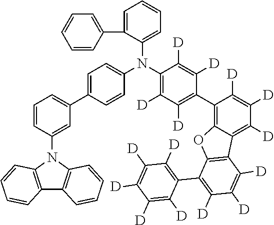 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5ccccc5-c5ccccc5)c([2H])c4[2H])c([2H])c([2H])c([2H])c23)c([2H])c1[2H]] | 4.34 | 53 | 99.90 | 820.41 |
| Compound 1-1045 | 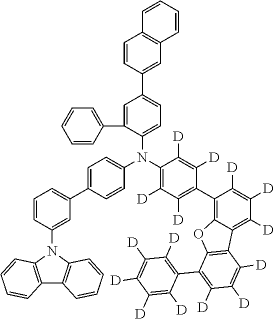 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5ccc(-c6ccc7ccccc7c6)cc5-c5ccccc5)c([2H])c4[2H])c([2H])c([2H])c([2H])c23)c([2H])c1[2H]] | 5.20 | 55 | 99.93 | 946.46 |
| Compound 1-1046 | 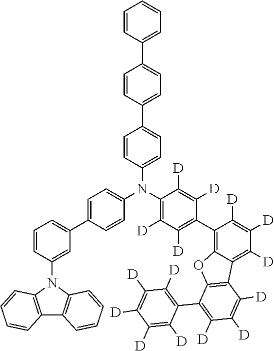 [SMILES: [2H]c1c([2H])c([2H])c(-c2c([2H])c([2H])c([2H])c3c2oc2c(-c4c([2H])c([2H])c(N(c5ccc(-c6ccc(-c7ccccc7)cc6)cc5)c5ccc(-c6cccc(-n7c8ccccc8c8ccccc87)c6)cc5)c([2H])c4[2H])c([2H])c([2H])c([2H])c23)c([2H])c1[2H]] | 5.37 | 60 | 99.95 | 896.44 |
| Compound 1-1047 | 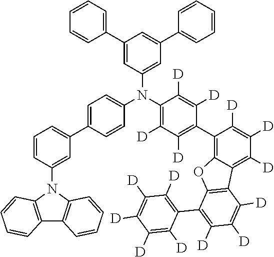 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5cc(-c6ccccc6)cc(-c6ccccc6)c5)c([2H])c4[2H])c([2H])c([2H])c([2H])c23)c([2H])c1[2H]] | 5.46 | 61 | 99.94 | 896.45 |
| Compound 1-1048 | 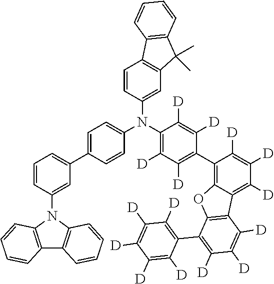 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5ccc6c(c5)C(C)(C)c5ccccc5-6)c([2H])c4[2H])c([2H])c([2H])c([2H])c23)c([2H])c1[2H]] | 5.59 | 65 | 99.98 | 860.44 |
| Compound 1-1049 | 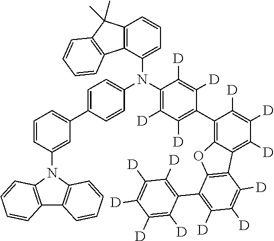 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5cccc6c5-c5ccccc5C6(C)C)c([2H])c4[2H])c([2H])c([2H])c([2H])c23)c([2H])c1[2H]] | 5.50 | 64 | 99.97 | 860.44 |
| Compound 1-1050 | 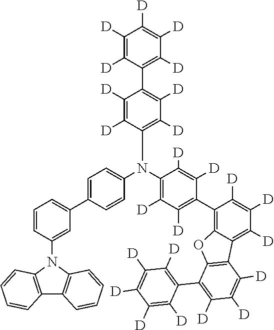 [SMILES: [2H]c1c([2H])c([2H])c(-c2c([2H])c([2H])c(N(c3ccc(-c4cccc(-n5c6ccccc6c6ccccc65)c4)cc3)c3c([2H])c([2H])c(-c4c([2H])c([2H])c([2H])c5c4oc4c(-c6c([2H])c([2H])c([2H])c([2H])c6[2H])c([2H])c([2H])c([2H])c45)c([2H])c3[2H])c([2H])c2[2H])c([2H])c1[2H]] | 4.89 | 59 | 99.92 | 829.47 |
| Compound 1-1051 | 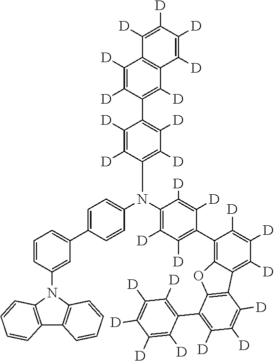 [SMILES: [2H]c1c([2H])c([2H])c(-c2c([2H])c([2H])c([2H])c3c2oc2c(-c4c([2H])c([2H])c(N(c5ccc(-c6cccc(-n7c8ccccc8c8ccccc87)c6)cc5)c5c([2H])c([2H])c(-c6c([2H])c([2H])c7c([2H])c([2H])c([2H])c([2H])c7c6[2H])c([2H])c5[2H])c([2H])c4[2H])c([2H])c([2H])c([2H])c23)c([2H])c1[2H]] | 5.28 | 60 | 99.96 | 881.50 |
| Compound 2-226 | 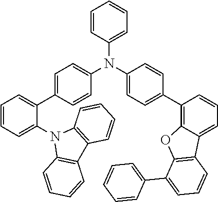 [SMILES: c1ccc(-c2cccc3c2oc2c(-c4ccc(N(c5ccccc5)c5ccc(-c6ccccc6-n6c7ccccc7c7ccccc76)cc5)cc4)cccc23)cc1] | 4.22 | 58 | 99.92 | 729.30 |
| Compound 2-231 | 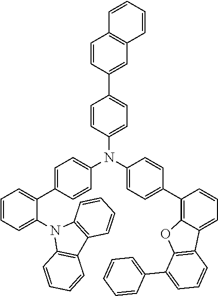 [SMILES: c1ccc(-c2cccc3c2oc2c(-c4ccc(N(c5ccc(-c6ccc7ccccc7c6)cc5)c5ccc(-c6ccccc6-n6c7ccccc7c7ccccc76)cc5)cc4)cccc23)cc1] | 5.81 | 68 | 99.95 | 855.33 |
| Compound 2-232 | 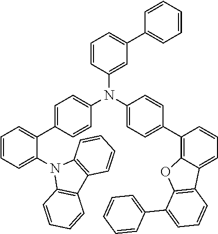 [SMILES: c1ccc(-c2cccc(N(c3ccc(-c4ccccc4-n4c5ccccc5c5ccccc54)cc3)c3ccc(-c4cccc5c4oc4c(-c6ccccc6)cccc45)cc3)c2)cc1] | 4.66 | 58 | 99.90 | 805.32 |
| Compound 2-236 | 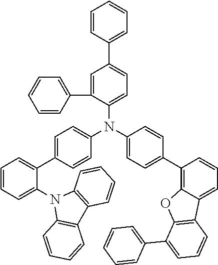 [SMILES: c1ccc(-c2ccc(N(c3ccc(-c4ccccc4-n4c5ccccc5c5ccccc54)cc3)c3ccc(-c4cccc5c4oc4c(-c6ccccc6)cccc45)cc3)c(-c3ccccc3)c2)cc1] | 4.84 | 55 | 99.95 | 881.35 |
| Compound 2-238 | 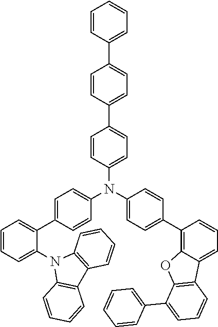 [SMILES: c1ccc(-c2ccc(-c3ccc(N(c4ccc(-c5ccccc5-n5c6ccccc6c6ccccc65)cc4)c4ccc(-c5cccc6c5oc5c(-c7ccccc7)cccc56)cc4)cc3)cc2)cc1] | 4.75 | 54 | 99.94 | 881.35 |
| Compound 2-243 | 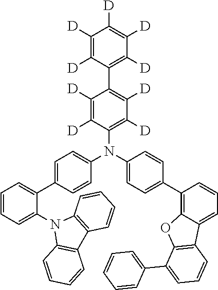 [SMILES: [2H]c1c([2H])c([2H])c(-c2c([2H])c([2H])c(N(c3ccc(-c4ccccc4-n4c5ccccc5c5ccccc54)cc3)c3ccc(-c4cccc5c4oc4c(-c6ccccc6)cccc45)cc3)c([2H])c2[2H])c([2H])c1[2H]] | 4.23 | 52 | 99.97 | 814.37 |
| Compound 2-244 | 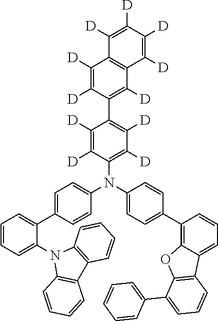 [SMILES: [2H]c1c([2H])c(N(c2ccc(-c3ccccc3-n3c4ccccc4c4ccccc43)cc2)c2ccc(-c3cccc4c3oc3c(-c5ccccc5)cccc34)cc2)c([2H])c([2H])c1-c1c([2H])c([2H])c2c([2H])c([2H])c([2H])c([2H])c2c1[2H]] | 5.19 | 60 | 99.91 | 866.41 |
| Compound 2-245 | 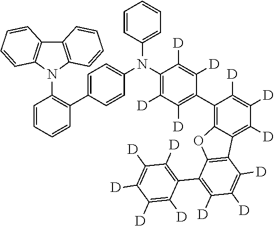 [SMILES: [2H]c1c([2H])c([2H])c(-c2c([2H])c([2H])c([2H])c3c2oc2c(-c4c([2H])c([2H])c(N(c5ccccc5)c5ccc(-c6ccccc6-n6c7ccccc7c7ccccc76)cc5)c([2H])c4[2H])c([2H])c([2H])c([2H])c23)c([2H])c1[2H]] | 3.64 | 49 | 99.94 | 744.38 |
| Compound 2-246 | 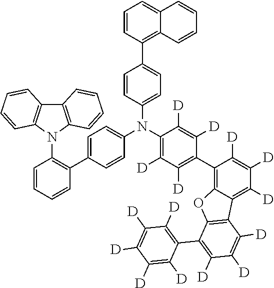 [SMILES: [2H]c1c([2H])c([2H])c(-c2c([2H])c([2H])c([2H])c3c2oc2c(-c4c([2H])c([2H])c(N(c5ccc(-c6ccccc6-n6c7ccccc7c7ccccc76)cc5)c5ccc(-c6cccc7ccccc67)cc5)c([2H])c4[2H])c([2H])c([2H])c([2H])c23)c([2H])c1[2H]] | 4.87 | 56 | 99.91 | 870.43 |
| Compound 2-247 | 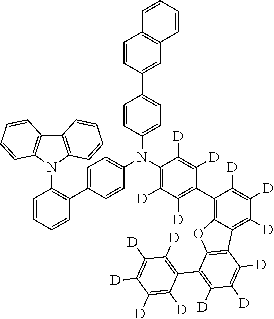 [SMILES: [2H]c1c([2H])c([2H])c(-c2c([2H])c([2H])c([2H])c3c2oc2c(-c4c([2H])c([2H])c(N(c5ccc(-c6ccc7ccccc7c6)cc5)c5ccc(-c6ccccc6-n6c7ccccc7c7ccccc76)cc5)c([2H])c4[2H])c([2H])c([2H])c([2H])c23)c([2H])c1[2H]] | 4.69 | 54 | 99.96 | 870.44 |
| Compound 2-248 | 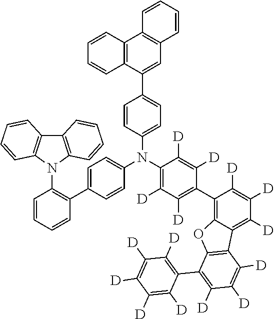 [SMILES: [2H]c1c([2H])c([2H])c(-c2c([2H])c([2H])c([2H])c3c2oc2c(-c4c([2H])c([2H])c(N(c5ccc(-c6ccccc6-n6c7ccccc7c7ccccc76)cc5)c5ccc(-c6cc7ccccc7c7ccccc67)cc5)c([2H])c4[2H])c([2H])c([2H])c([2H])c23)c([2H])c1[2H]] | 5.98 | 65 | 99.92 | 920.44 |
| Compound 2-249 | 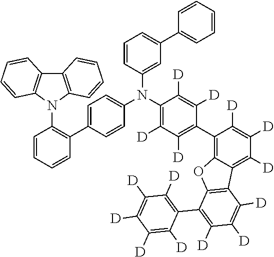 [SMILES: [2H]c1c([2H])c([2H])c(-c2c([2H])c([2H])c([2H])c3c2oc2c(-c4c([2H])c([2H])c(N(c5ccc(-c6ccccc6-n6c7ccccc7c7ccccc76)cc5)c5cccc(-c6ccccc6)c5)c([2H])c4[2H])c([2H])c([2H])c([2H])c23)c([2H])c1[2H]] | 5.00 | 61 | 99.93 | 820.41 |
| Compound 2-253 | 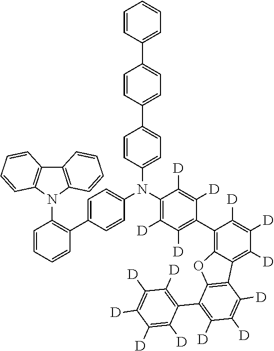 [SMILES: [2H]c1c([2H])c([2H])c(-c2c([2H])c([2H])c([2H])c3c2oc2c(-c4c([2H])c([2H])c(N(c5ccc(-c6ccc(-c7ccccc7)cc6)cc5)c5ccc(-c6ccccc6-n6c7ccccc7c7ccccc76)cc5)c([2H])c4[2H])c([2H])c([2H])c([2H])c23)c([2H])c1[2H]] | 5.01 | 56 | 99.95 | 896.44 |
| Compound 2-254 | 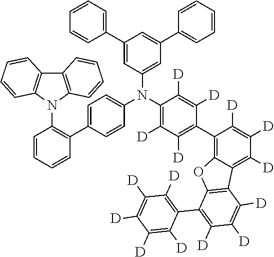 [SMILES: [2H]c1c([2H])c([2H])c(-c2c([2H])c([2H])c([2H])c3c2oc2c(-c4c([2H])c([2H])c(N(c5ccc(-c6ccccc6-n6c7ccccc7c7ccccc76)cc5)c5cc(-c6ccccc6)cc(-c6ccccc6)c5)c([2H])c4[2H])c([2H])c([2H])c([2H])c23)c([2H])c1[2H]] | 4.65 | 52 | 99.91 | 896.44 |
| Compound 2-255 | 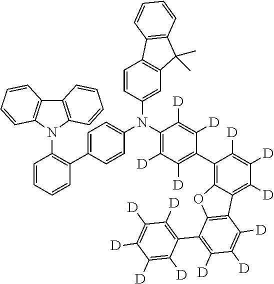 [SMILES: [2H]c1c([2H])c([2H])c(-c2c([2H])c([2H])c([2H])c3c2oc2c(-c4c([2H])c([2H])c(N(c5ccc(-c6ccccc6-n6c7ccccc7c7ccccc76)cc5)c5ccc6c(c5)C(C)(C)c5ccccc5-6)c([2H])c4[2H])c([2H])c([2H])c([2H])c23)c([2H])c1[2H]] | 4.30 | 50 | 99.98 | 860.45 |
| Compound 2-257 | 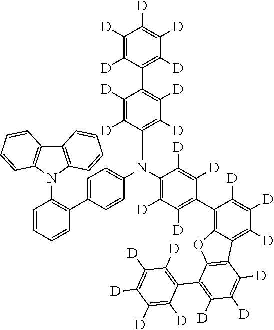 [SMILES: [2H]c1c([2H])c([2H])c(-c2c([2H])c([2H])c(N(c3ccc(-c4ccccc4-n4c5ccccc5c5ccccc54)cc3)c3c([2H])c([2H])c(-c4c([2H])c([2H])c([2H])c5c4oc4c(-c6c([2H])c([2H])c([2H])c([2H])c6[2H])c([2H])c([2H])c([2H])c45)c([2H])c3[2H])c([2H])c2[2H])c([2H])c1[2H]] | 4.72 | 57 | 99.92 | 829.47 |
| Compound 2-258 | 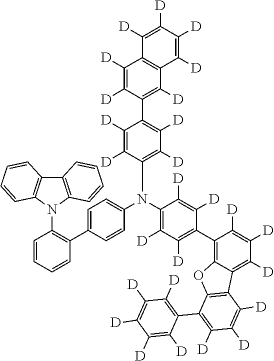 [SMILES: [2H]c1c([2H])c([2H])c(-c2c([2H])c([2H])c([2H])c3c2oc2c(-c4c([2H])c([2H])c(N(c5ccc(-c6ccccc6-n6c7ccccc7c7ccccc76)cc5)c5c([2H])c([2H])c(-c6c([2H])c([2H])c7c([2H])c([2H])c([2H])c([2H])c7c6[2H])c([2H])c5[2H])c([2H])c4[2H])c([2H])c([2H])c([2H])c23)c([2H])c1[2H]] | 5.37 | 61 | 99.95 | 881.50 |

### Example 1: Manufacturing 1 of Organic Electroluminescent Device

A positive electrode was formed with ITO on a substrate on which a reflective layer was formed and surface-treated with N₂ plasma or UV-ozone. HAT-CN was deposited above the positive electrode in a thickness of 10 nm as a hole injection layer (HIL). Subsequently, a hole transport layer (HTL) was formed by depositing N4,N4,N4′,N4′-tetra([1,1′-biphenyl]-4-yl)-[1,1′-biphenyl]-4,4′-diamine in a thickness of 110 nm.

A hole transport auxiliary layer was formed above the hole transport layer by forming Compound 1-97 by vacuum deposition in a thickness of 15 nm, and N1,N1,N6,N6-tetrakis(4-(1-silyl)phenyl)piren-1,6-diamine as a dopant was doped at about 3 wt % while 9,10-bis(2-naphthyl)anthracene (9,10-Bis(2-naphthyl)anthracene, ADN) capable of forming a blue EML as a light emitting layer (EML) was deposited above the hole transport auxiliary layer in a thickness of 25 nm.

An electron transport layer (ETL) was deposited above the light emitting layer (EML) in a thickness of 30 nm by mixing anthracene derivative and LiQ at a mass ratio of 1:1, and LiQ as an electron injection layer (EIL) was deposited above the electron transport layer (ETL) in a thickness of 1 nm. Then, a mixture mixing magnesium and silver (Ag) at 9:1 was deposited in a thickness of 15 nm as a negative electrode, and N4,N4′-bis[4-[bis(3-Methylphenyl)amino]phenyl]-N4,N4′-diphenyl-[1,1′-biphenyl]-4,4′-diamine (DNTPD) as a capping layer was deposited above the negative electrode in a thickness of 60 nm. An organic electroluminescent device was manufactured by attaching a seal cap containing a moisture absorbent on the capping layer using a UV curing adhesive to protect the organic electroluminescent device from O₂ or moisture in the atmosphere.

### Examples 2 to 37

An organic electroluminescent device was manufactured in the same manner as Example 1 except that the compounds listed in Table 11 below were used instead of Compound 1-97 as the hole transport auxiliary layer in Example 1.

### Comparative Examples 1 to 4

An organic electroluminescent device was manufactured in the same manner as Example 1 except that Compounds A to D were used instead of Compound 1-97 as the hole transport auxiliary layer in Example 1.

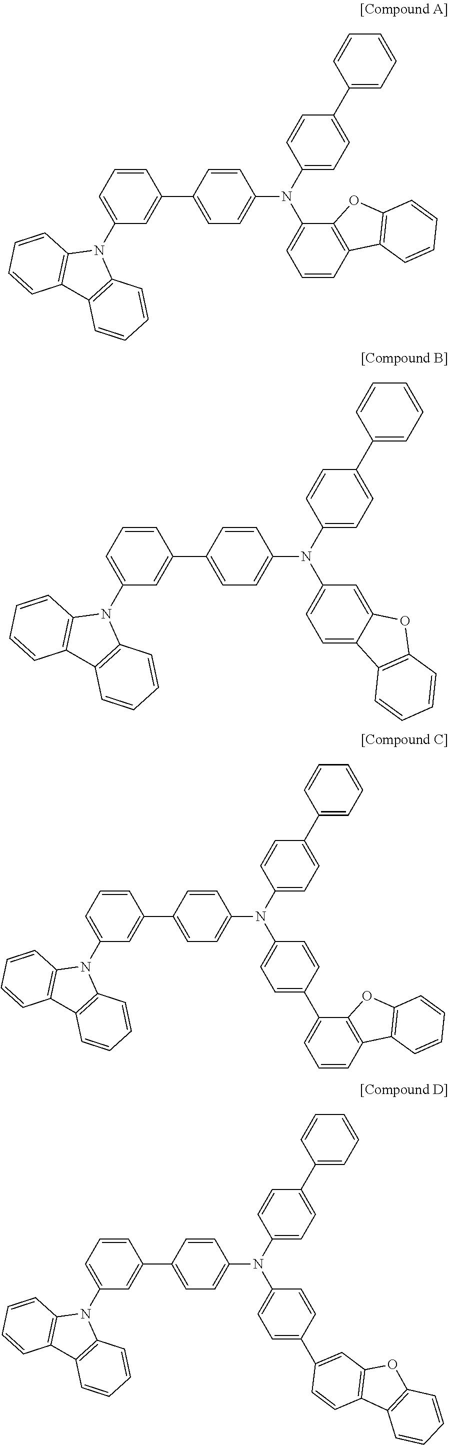 [SMILES: c1ccc(-c2ccc(N(c3ccc(-c4cccc(-n5c6ccccc6c6ccccc65)c4)cc3)c3cccc4c3oc3ccccc34)cc2)cc1 ; c1ccc(-c2ccc(N(c3ccc(-c4cccc(-n5c6ccccc6c6ccccc65)c4)cc3)c3ccc4c(c3)oc3ccccc34)cc2)cc1 ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1 ; c1ccc(-c2ccc(N(c3ccc(-c4cccc(-n5c6ccccc6c6ccccc65)c4)cc3)c3ccc(-c4ccc5c(c4)oc4ccccc45)cc3)cc2)cc1]

### Experimental Example 1: Device Performance Analysis

For the organic electroluminescent devices manufactured in Examples and Comparative Examples, the electro-optical characteristics of the devices were analyzed under the condition of a constant current of 10 mA/cm², and the lifetime was measured under a driving condition of 20 mA/cm².

The experimental result is expressed in Table 11 below.

TABLE 11

| | | Driving | | | | | |
|---|---|---|---|---|---|---|---|
| | Hole transport | voltage | Efficiency | | Color | | Lifetime |
| Items | auxiliary layer | (V) | Cd/A | EQE | CIEx | CIEy | T95(hrs) |
| Example 1 | Compound 1-97 | 3.51 | 8.1 | 17.1 | 0.138 | 0.046 | 360 |
| Example 2 | Compound 1-121 | 3.51 | 8.2 | 17.0 | 0.138 | 0.046 | 320 |
| Example 3 | Compound 1-827 | 3.55 | 8.6 | 18.2 | 0.138 | 0.045 | 330 |
| Example 4 | Compound 1-153 | 3.52 | 8.4 | 17.6 | 0.138 | 0.046 | 290 |
| Example 5 | Compound 1-313 | 3.54 | 8.3 | 17.5 | 0.138 | 0.046 | 360 |
| Example 6 | Compound 1-217 | 3.58 | 8.2 | 17.2 | 0.138 | 0.046 | 310 |
| Example 7 | Compound 1-505 | 3.68 | 9.5 | 19.0 | 0.136 | 0.049 | 350 |
| Example 8 | Compound 1-701 | 3.70 | 9.1 | 18.1 | 0.136 | 0.049 | 290 |
| Example 9 | Compound 1-702 | 3.68 | 9.0 | 18.2 | 0.136 | 0.049 | 350 |
| Example 10 | Compound 1-703 | 3.56 | 9.1 | 18.6 | 0.137 | 0.048 | 360 |
| Example 11 | Compound 1-704 | 3.78 | 8.8 | 18.0 | 0.137 | 0.048 | 350 |
| Example 12 | Compound 1-705 | 3.68 | 8.8 | 17.9 | 0.136 | 0.048 | 350 |
| Example 13 | Compound 1-706 | 3.72 | 8.0 | 17.3 | 0.140 | 0.044 | 390 |
| Example 14 | Compound 1-707 | 3.71 | 9.1 | 18.3 | 0.136 | 0.049 | 400 |
| Example 15 | Compound 1-823 | 3.54 | 9.0 | 18.1 | 0.137 | 0.049 | 350 |
| Example 16 | Compound 1-845 | 3.73 | 8.1 | 17.3 | 0.139 | 0.044 | 380 |
| Example 17 | Compound 1-917 | 3.69 | 9.0 | 17.5 | 0.135 | 0.051 | 375 |
| Example 18 | Compound 1-973 | 3.58 | 8.9 | 17.8 | 0.137 | 0.049 | 365 |
| Example 19 | Compound 1-1003 | 3.64 | 9.3 | 19.1 | 0.137 | 0.048 | 405 |
| Example 20 | Compound 1-127 | 3.55 | 8.4 | 17.6 | 0.138 | 0.046 | 340 |
| Example 21 | Compound 1-129 | 3.51 | 8.9 | 17.9 | 0.137 | 0.047 | 320 |
| Example 22 | Compound 1-487 | 3.51 | 8.8 | 17.8 | 0.136 | 0.049 | 310 |
| Example 23 | Compound 1-709 | 3.53 | 8.0 | 17.1 | 0.138 | 0.045 | 330 |
| Example 24 | Compound 1-712 | 3.57 | 9.1 | 18.4 | 0.137 | 0.049 | 355 |
| Example 25 | Compound 1-714 | 3.61 | 9.7 | 18.5 | 0.134 | 0.053 | 325 |
| Example 26 | Compound 1-135 | 3.59 | 9.3 | 18.7 | 0.137 | 0.049 | 345 |
| Example 27 | Compound 1-229 | 3.58 | 9.3 | 18.2 | 0.135 | 0.051 | 330 |
| Example 28 | Compound 1-414 | 3.59 | 8.6 | 17.9 | 0.137 | 0.047 | 315 |
| Example 29 | Compound 1-147 | 3.59 | 8.7 | 18.0 | 0.138 | 0.047 | 325 |
| Example 30 | Compound 1-169 | 3.60 | 8.7 | 18.3 | 0.138 | 0.046 | 335 |
| Example 31 | Compound 1-1008 | 3.59 | 9.1 | 18.6 | 0.137 | 0.048 | 350 |
| Example 32 | Compound 1-1009 | 3.56 | 8.9 | 18.3 | 0.137 | 0.048 | 360 |
| Example 33 | Compound 1-1010 | 3.59 | 8.8 | 18.1 | 0.137 | 0.047 | 355 |
| Example 34 | Compound 1-1016 | 3.45 | 9.5 | 19.2 | 0.137 | 0.049 | 395 |
| Example 35 | Compound 1-1019 | 3.57 | 8.9 | 18.0 | 0.135 | 0.049 | 328 |
| Example 36 | Compound 1-1024 | 3.51 | 8.9 | 17.9 | 0.136 | 0.049 | 343 |
| Example 37 | Compound 1-1036 | 3.50 | 9.6 | 19.0 | 0.135 | 0.050 | 392 |
| Comparative | [Compound A] | 3.85 | 6.9 | 15.5 | 0.140 | 0.042 | 132 |
| Example 1 | | | | | | | |
| Comparative | [Compound B] | 3.90 | 6.4 | 14.2 | 0.141 | 0.042 | 125 |
| Example 2 | | | | | | | |
| Comparative | [Compound C] | 3.95 | 7.3 | 15.8 | 0.140 | 0.043 | 175 |
| Example 3 | | | | | | | |
| Comparative | [Compound D] | 3.80 | 6.3 | 14.1 | 0.141 | 0.042 | 197 |
| Example 4 | | | | | | | |

According to the experimental result in Table 11, it was confirmed that when the compounds of the present invention were used as a material for the hole transport auxiliary layer of the organic electroluminescent device, the driving voltage was low, and excellent device efficiency characteristics and long lifetime characteristics were shown as compared to Comparative Examples.

### Example 38: Manufacturing 2 of Organic Electroluminescent Device

A positive electrode was formed with ITO on a substrate on which a reflective layer was formed and surface-treated with N₂ plasma or UV-ozone. HAT-CN was deposited above the positive electrode in a thickness of 10 nm as a hole injection layer (HIL). Subsequently, a hole transport layer (HTL) was formed by depositing N4,N4,N4′,N4′-tetra([1,1′-biphenyl]-4-yl)-[1,1′-biphenyl]-4,4′-diamine in a thickness of 110 nm.

A hole transport auxiliary layer was formed above the hole transport layer by forming Compound 2-25 by vacuum deposition in a thickness of 15 nm, and N1,N1,N6,N6-tetrakis(4-(1-silyl)phenyl)piren-1,6-diamine as a dopant was doped at about 3 wt % while 9,10-bis(2-naphthyl)anthracene (9,10-Bis(2-naphthyl)anthracene, ADN) capable of forming a blue EML as a light emitting layer (EML) was deposited above the hole transport auxiliary layer in a thickness of 25 nm.

An electron transport layer (ETL) was deposited in a thickness of 30 nm by mixing anthracene derivative and LiQ at a mass ratio of 1:1, and LiQ as an electron injection layer (EIL) was deposited above the electron transport layer (ETL) in a thickness of 1 nm. Then, a mixture mixing magnesium and silver (Ag) at 9:1 was deposited in a thickness of 15 nm as a negative electrode, and N4,N4′-bis[4-[bis(3-Methylphenyl)amino]phenyl]-N4,N4′-diphenyl-[1,1′-biphenyl]-4,4′-diamine (DNTPD) as a capping layer was deposited above the negative electrode in a thickness of 60 nm. An organic electroluminescent device was manufactured by attaching a seal cap containing a moisture absorbent on the capping layer using a UV curing adhesive to protect the organic electroluminescent device from O₂ or moisture in the atmosphere.

### Examples 39 to 57

An organic electroluminescent device was manufactured in the same manner as Example 38 except that the compounds listed in Table 12 below were used instead of Compound 2-25 as the hole transport auxiliary layer in Example 38.

### Comparative Examples 5 to 8

An organic electroluminescent device was manufactured in the same manner as Example 38 except that Compounds E to H were used instead of Compound 2-25 as the hole transport auxiliary layer in Example 38.

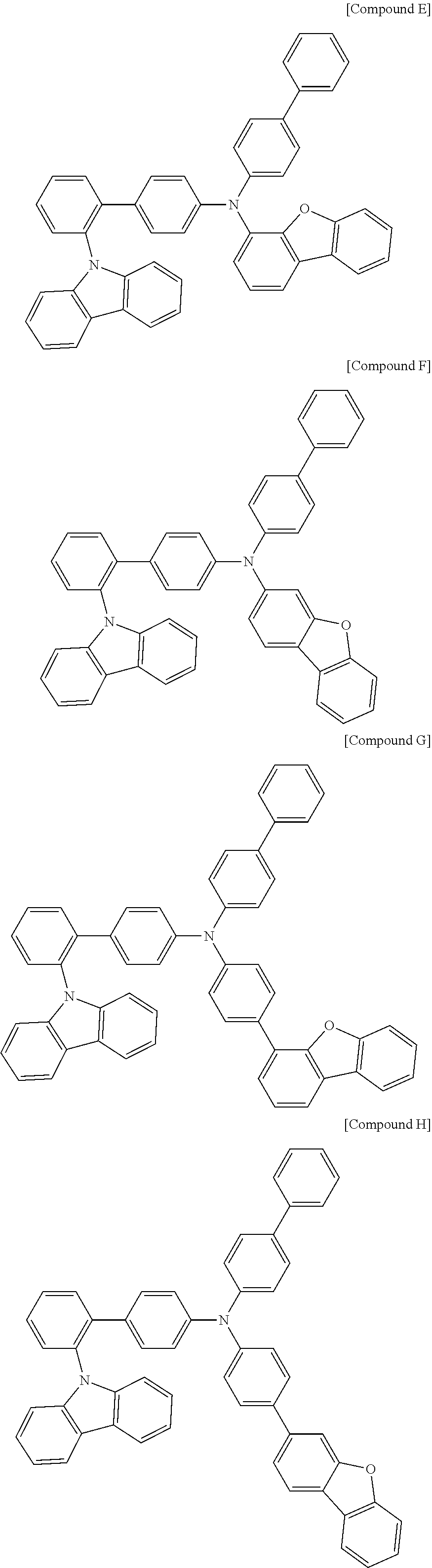

### Experimental Example 2: Device Performance Analysis

For the organic electroluminescent devices manufactured in Examples and Comparative Examples, the electro-optical characteristics of the devices were analyzed under the condition of a constant current of 10 mA/cm², and the lifetime was measured under a driving condition of 20 mA/cm².

The experimental result is expressed in Table 12 below.

TABLE 12

| | | Driving | | | | | |
|---|---|---|---|---|---|---|---|
| | Hole transport | voltage | Efficiency | | Color | | Lifetime |
| Items | auxiliary layer | (V) | Cd/A | EQE | CIEx | CIEy | T95(hrs) |
| Example 38 | Compound 2-25 | 3.51 | 10.0 | 18.5 | 0.134 | 0.055 | 290 |
| Example 39 | Compound 2-31 | 3.48 | 9.7 | 18.4 | 0.134 | 0.054 | 315 |
| Example 40 | Compound 2-1 | 3.55 | 8.3 | 17.5 | 0.139 | 0.045 | 380 |
| Example 41 | Compound 2-27 | 3.53 | 8.8 | 17.9 | 0.137 | 0.048 | 297 |
| Example 42 | Compound 2-49 | 3.54 | 8.8 | 17.8 | 0.137 | 0.048 | 310 |
| Example 43 | Compound 2-170 | 3.59 | 8.7 | 17.8 | 0.137 | 0.048 | 295 |
| Example 44 | Compound 2-173 | 3.65 | 8.6 | 17.5 | 0.137 | 0.048 | 323 |
| Example 45 | Compound 2-37 | 3.62 | 9.0 | 17.9 | 0.136 | 0.050 | 312 |
| Example 46 | Compound 2-61 | 3.67 | 9.2 | 18.3 | 0.136 | 0.049 | 300 |
| Example 47 | Compound 2-182 | 3.56 | 9.3 | 18.5 | 0.136 | 0.050 | 360 |
| Example 48 | Compound 2-31 | 3.51 | 8.5 | 17.5 | 0.139 | 0.044 | 298 |
| Example 49 | Compound 2-33 | 3.53 | 8.9 | 18.2 | 0.137 | 0.048 | 314 |
| Example 50 | Compound 2-55 | 3.54 | 8.8 | 18.2 | 0.137 | 0.047 | 321 |
| Example 51 | Compound 2-214 | 3.58 | 9.3 | 18.7 | 0.137 | 0.049 | 380 |
| Example 52 | Compound 2-215 | 3.55 | 9.2 | 18.0 | 0.136 | 0.051 | 375 |
| Example 53 | Compound 2-217 | 3.57 | 9.5 | 18.3 | 0.135 | 0.052 | 390 |
| Example 54 | Compound 2-223 | 3.46 | 9.2 | 18.2 | 0.138 | 0.046 | 420 |
| Example 55 | Compound 2-225 | 3.49 | 9.6 | 18.9 | 0.137 | 0.048 | 430 |
| Example 56 | Compound 2-244 | 3.50 | 10.0 | 19.1 | 0.135 | 0.053 | 425 |
| Example 57 | Compound 2-257 | 3.56 | 97 | 18.5 | 0.134 | 0.053 | 452 |
| Comparative | Compound E | 3.82 | 7.4 | 16.0 | 0.139 | 0.045 | 185 |
| Example 5 | | | | | | | |
| Comparative | Compound F | 3.80 | 7.3 | 16.4 | 0.138 | 0.045 | 196 |
| Example 6 | | | | | | | |
| Comparative | Compound G | 3.89 | 7.6 | 16.5 | 0.139 | 0.045 | 187 |
| Example 7 | | | | | | | |
| Comparative | Compound H | 3.77 | 7.7 | 16.6 | 0.139 | 0.045 | 198 |
| Example 8 | | | | | | | |

According to the experimental result in Table 12, it was confirmed that when the compounds of the present invention were used as a material for the hole transport auxiliary layer of the organic electroluminescent device, the driving voltage was low, and excellent device efficiency characteristics and long lifetime characteristics were shown as compared to Comparative Examples.

Although the exemplary embodiments of the present invention have been described above in detail, the scope of the present invention is not limited thereto, and various modifications and improvements of those skilled in the art using the basic concept of the present invention defined in the appended claims are also included in the scope of the present invention.

1. A compound represented by Chemical Formula 1: 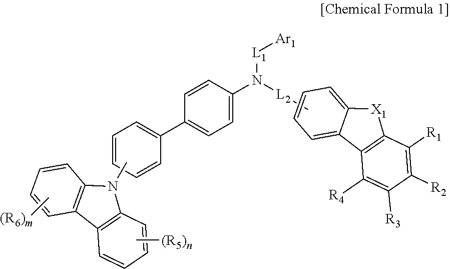
wherein,
n and m is an integer of 0 to 4,
X₁ is selected from the group consisting of O, N(R₇), C(R₈)(R₉), and S,
L₁ and L₂ are the same as or different from each other and are each independently selected from the group consisting of a single bond, a substituted or unsubstituted arylene group having 6 to 30 carbon atoms, a substituted or unsubstituted heteroarylene group having 1 to 30 carbon atoms, a substituted or unsubstituted alkylene group having 1 to 10 carbon atoms, a substituted or unsubstituted cycloalkylene group having 3 to 10 carbon atoms, a substituted or unsubstituted alkenylene group with 2 to 10 carbon atoms, a substituted or unsubstituted cycloalkenylene group having 3 to 10 carbon atoms, a substituted or unsubstituted heteroalkylene group having 1 to 10 carbon atoms, a substituted or unsubstituted heterocycloalkylene group having 2 to 10 carbon atoms, a substituted or unsubstituted heteroalkenylene group having 2 to 10 carbon atoms, and a substituted or unsubstituted heterocycloalkenylene group having 2 to 10 carbon atoms,
Ar₁ is selected from the group consisting of a substituted or unsubstituted aryl group having 6 to 30 carbon atoms, a substituted or unsubstituted heteroaryl group having 1 to 30 carbon atoms, a substituted or unsubstituted alkyl group having 1 to 20 carbon atoms, a substituted or unsubstituted cycloalkyl group having 3 to 20 carbon atoms, a substituted or unsubstituted heteroalkyl group having 1 to 20 carbon atoms, a substituted or unsubstituted heterocycloalkyl group having 2 to 20 carbon atoms, a substituted or unsubstituted aralkyl group having 6 to 30 carbon atoms, a substituted or unsubstituted alkenyl group having 2 to 20 carbon atoms, a substituted or unsubstituted cycloalkenyl group having 3 to 20 carbon atoms, and a substituted or unsubstituted heteroalkenyl groups having 2 to 20 carbon atoms,
R₁ to R₉ are the same as or different from each other, are each independently selected from the group consisting of hydrogen, deuterium, a cyano group, a nitro group, a halogen group, a hydroxy group, a substituted or unsubstituted alkylthio group having 1 to 4 carbon atoms, a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted alkynyl group having 2 to 24 carbon atoms, a substituted or unsubstituted aralkyl group having 7 to 30 carbon atoms, a substituted or unsubstituted aryl group having 6 to 30 carbon atoms, a substituted or unsubstituted heteroaryl group having 1 to 60 carbon atoms, a substituted or unsubstituted heteroarylalkyl group having 2 to 30 carbon atoms, a substituted or unsubstituted alkoxy group having 1 to 30 carbon atoms, a substituted or unsubstituted alkylamino group having 1 to 30 carbon atoms, a substituted or unsubstituted arylamino group having 6 to 30 carbon atoms, a substituted or unsubstituted aralkylamino group having 7 to 30 carbon atoms, a substituted or unsubstituted heteroarylamino group having 1 to 24 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms, and are bonded with adjacent groups to form a substituted or unsubstituted ring, and
any one or more of R₁ to R₄ is bonded with adjacent groups to form a substituted or unsubstituted ring.

2. The compound of claim 1, wherein the compound represented by Chemical Formula 1 is expressed by Chemical Formulas 2 to 7 below: 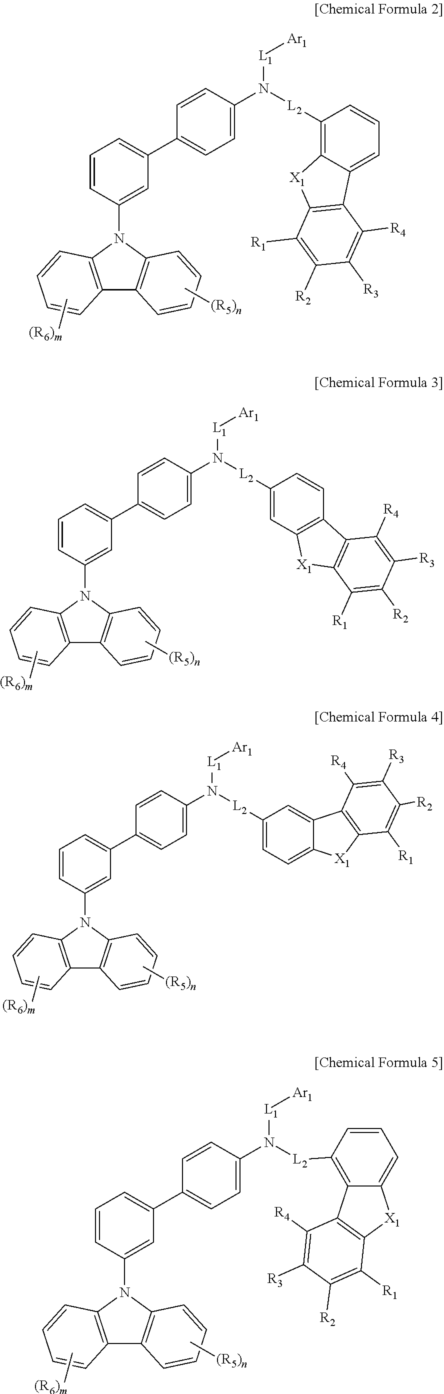 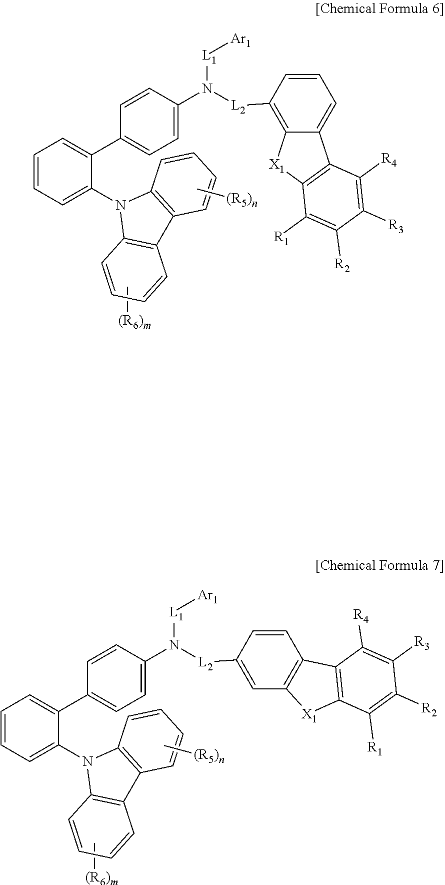
wherein,
n, m, L₁, L₂, Ar₁, X₁, and R₁ to R₆ are defined in claim 1.

3. The compound of claim 1, wherein the L₁ and L₂ are the same as or different from each other and are each independently a single bond or a substituted or unsubstituted arylene group having 6 to 30 carbon atoms.

4. The compound of claim 1, wherein X₁ is O or S.

5. The compound of claim 1, wherein Ar₁ is selected from the group consisting of Chemical Formulas 8 to 12 below: 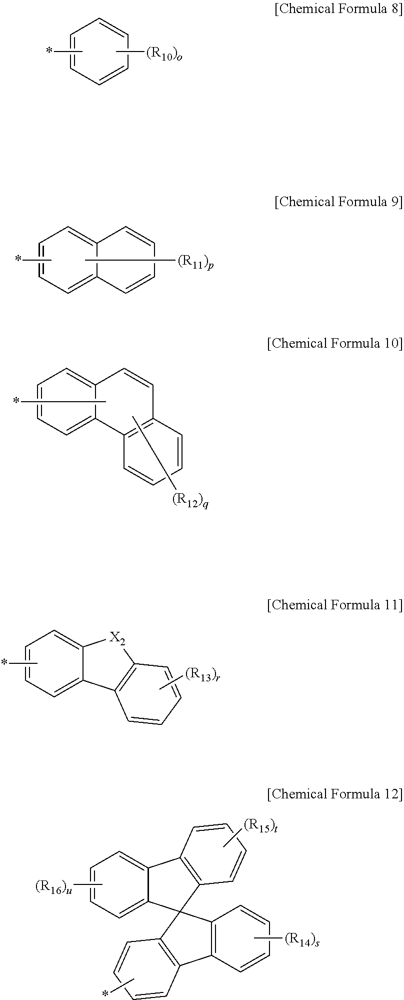
wherein,
* indicates a portion which is bonded,
o is an integer from 0 to 5,
p is an integer from 0 to 7,
q is an integer from 0 to 6,
r, s, t, and u are the same as or different from each other and are each independently an integer from 0 to 4,
X₂ is selected from the group consisting of O, N(R₁₇), C(R₁₈)(R₁₉), and S, and
R₁₀ to R₁₉ are the same as or different from each other, are each independently selected from the group consisting of hydrogen, deuterium, a cyano group, a nitro group, a halogen group, a hydroxy group, a substituted or unsubstituted alkylthio group having 1 to 4 carbon atoms, a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted alkynyl group having 2 to 24 carbon atoms, a substituted or unsubstituted aralkyl group having 7 to 30 carbon atoms, a substituted or unsubstituted aryl group having 6 to 30 carbon atoms, a substituted or unsubstituted heteroaryl group having 1 to 60 carbon atoms, a substituted or unsubstituted heteroarylalkyl group having 2 to 30 carbon atoms, a substituted or unsubstituted alkoxy group having 1 to 30 carbon atoms, a substituted or unsubstituted alkylamino group having 1 to 30 carbon atoms, a substituted or unsubstituted arylamino group having 6 to 30 carbon atoms, a substituted or unsubstituted aralkylamino group having 7 to 30 carbon atoms, a substituted or unsubstituted heteroarylamino group having 1 to 24 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms, and are bonded with adjacent groups to form a substituted or unsubstituted ring.

6. An organic electroluminescent device comprising: a first electrode; a second electrode provided to face the first electrode; and one or more organic layers provided between the first electrode and the second electrode,
wherein at least one of the one or more organic layers includes the compound of claim 1.

7. The organic electroluminescent device of claim 6, wherein the organic layer is selected from the group consisting of a hole injection layer, a hole transport layer, a hole transport auxiliary layer, a light emitting layer, an electron transport layer, and an electron injection layer.

8. The organic electroluminescent device of claim 6, wherein the organic layer is a hole transport auxiliary layer.
